(19) United States
(12) Patent Application Publication
(10) Pub. No.: US 2023/0200220 A1
(43) Pub. Date: Jun. 22, 2023

(54) LIGHT EMITTING ELEMENT AND AMINE COMPOUND FOR THE SAME

(71) Applicant: Samsung Display Co., Ltd., Yongin-si (KR)

(72) Inventor: Takuya UNO, Yokohama (JP)

(21) Appl. No.: 18/049,154

(22) Filed: Oct. 24, 2022

(30) Foreign Application Priority Data
Dec. 22, 2021 (KR) 10-2021-0184859

(51) Int. Cl. H01L 51/00; C07C 211/54; C07D 307/91; C07D 333/76; C07D 409/12; C07D 209/82; C07D 405/12; H01L 51/50

(52) U.S. Cl. CPC H01L 51/006; H01L 51/0052; C07C 211/54; C07D 307/91; H01L 51/0073; H01L 51/0061; H01L 51/0074; C07D 333/76; C07D 409/12; C07D 209/82; H01L 51/0072; C07D 405/12; H01L 51/5012; C07B 2200/05

(57) ABSTRACT

Provided are a light emitting element and an amine compound for the light emitting element, wherein the light emitting element includes a first electrode, a second electrode on the first electrode, and at least one functional layer between the first electrode and the second electrode, the functional layer includes the amine compound represented by the disclosed formula structure, and thus, the luminous efficiency and service life of the light emitting element may be improved.

### CROSS-REFERENCE TO RELATED APPLICATION

This application claims priority to and the benefit of Korean Patent Application No. 10-2021-0184859, filed on Dec. 22, 2021, the entire contents of which are hereby incorporated by reference.

### BACKGROUND

### 1. Field

Embodiments of the present disclosure herein relate to a light emitting element and an amine compound for the same, and, for example, to a light emitting element including an amine compound in a hole transport region.

### 2. Related Art

Recently, the development of an organic electroluminescence display device as an image display device is being actively conducted. The organic electroluminescence display device includes a so-called self-luminescent light emitting element in which holes and electrons injected from a first electrode and a second electrode, respectively, recombine in an emission layer, and thus, a luminescent material of the emission layer emits light to implement display.

In the application of a light emitting element to a display device, there is a demand for a light emitting element having low driving voltage, high luminous efficiency, and a long service life, and development of materials for a light emitting element capable of stably attaining such characteristics is being continuously conducted.

In addition, development of materials of a hole transport region for suppressing or reducing the diffusion of exciton energy of the emission layer is being carried out in order to implement a highly efficient light emitting element.

### SUMMARY

Embodiments of the present disclosure provide a light emitting element exhibiting excellent luminous efficiency and long service life characteristics.

Embodiments of the present disclosure also provide an amine compound which is a material for a light emitting element having high efficiency and long service life characteristics.

An embodiment of the present disclosure provides an amine compound represented by Formula 1 below:

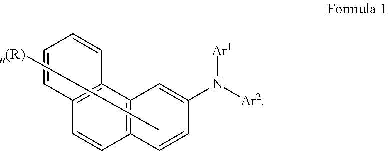

In Formula 1 above, R is a hydrogen atom, a deuterium atom, a halogen atom, or a substituted or unsubstituted alkyl group having 1 to 20 carbon atoms, n is an integer of 0 to 9, and Ar¹ and Ar² are each independently represented any one selected from among by Formula 2 to Formula 5 below:

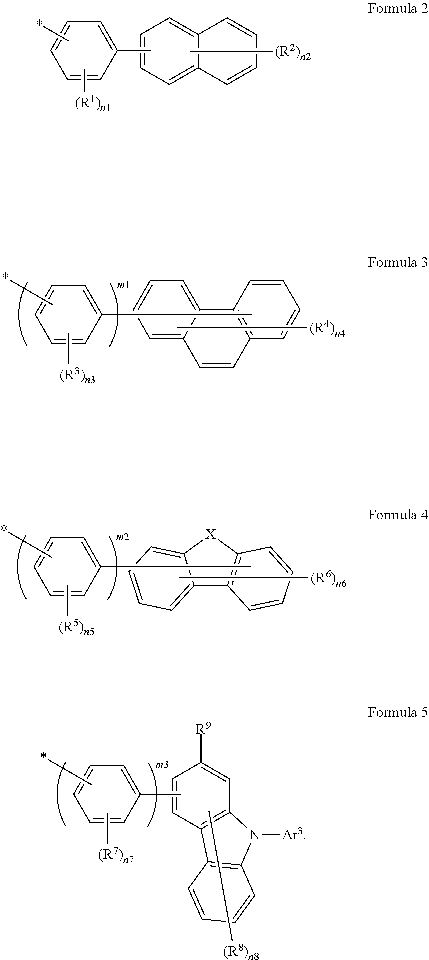

In Formula 4 above, X is O or S, and in Formula 5 above, Ar³ is a substituted or unsubstituted phenyl group.

In Formula 2 to Formula 5 above, R¹ to R⁵, R⁷, and R⁹ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, R⁶ and R⁸ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, or adjacent R⁶'s or adjacent R⁸'s are bonded to each other to form an aromatic ring, n1, n3, n5, and n7 are each independently an integer of 0 to 4, n2 and n6 are each independently an integer of 0 to 7, n4 is an integer of 0 to 9, n8 is an integer of 0 to 6, m1 to m3 are each independently 0 or 1, when Ar¹ and Ar² are each independently represented by any one selected from among Formula 3 to Formula 5 above, at least one selected from among m1 to m3 is 1, the case where both Ar¹ and Ar² are represented by Formula 3 above is excluded, when both Ar¹ and Ar² are represented by Formula 4 above, any one selected from among two m2's is 1, and the other is 0, and a structure in which any hydrogen atom in the molecule is substituted with a deuterium atom is included.

In an embodiment, Formula 2 above may be represented by Formula 2-1 or Formula 2-2 below:

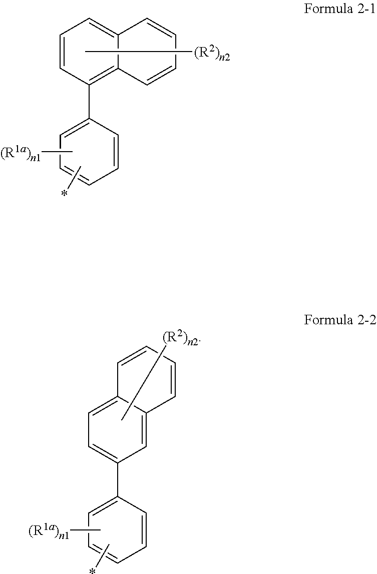

In Formula 2-1 and Formula 2-2 above, R^{1a} is a hydrogen atom, a deuterium atom, or a substituted or unsubstituted phenyl group, n1, n2, and R² are the same as defined with respect to Formula 2 above.

In an embodiment, Formula 2-1 above may be represented by 2-a or 2-b below, and Formula 2-2 above may be represented by 2-c or 2-d below.

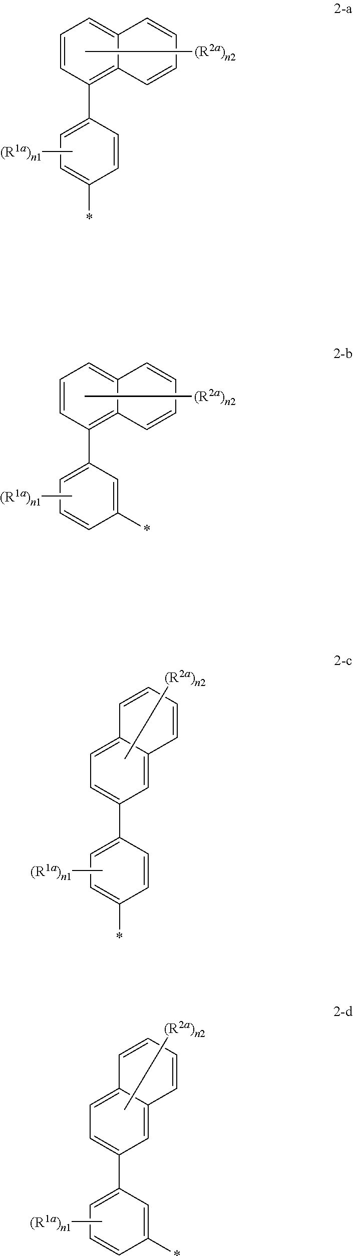

In 2-a to 2-d above, R^{2a} is a hydrogen atom, a deuterium atom, or a substituted or unsubstituted aryl group having 6 to 15 ring-forming carbon atoms, and n1, n2, and R^{1a} are the same as defined with respect to Formula 2-1 and Formula 2-2 above.

In an embodiment, Formula 3 above may be represented by any one selected from among Formula 3-1 to Formula 3-5 below:

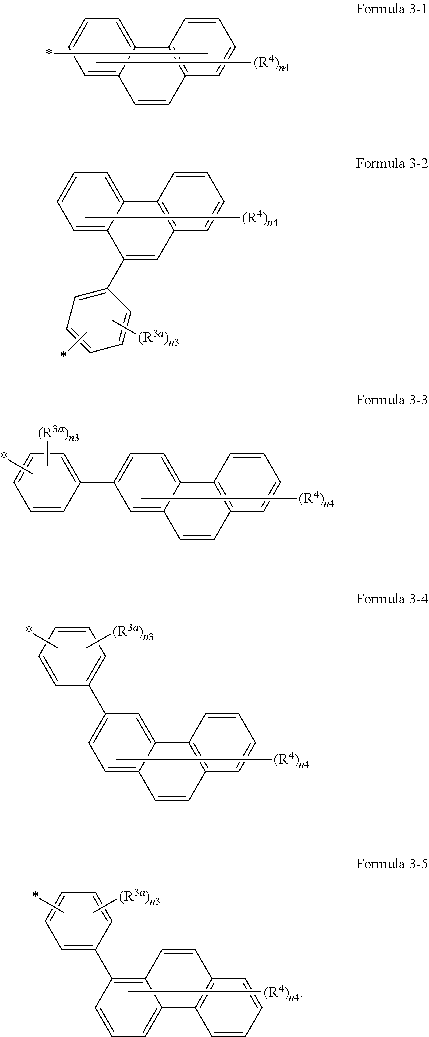

In Formula 3-1 to Formula 3-5 above, R^{3a} is a hydrogen atom, a deuterium atom, or a substituted or unsubstituted phenyl group, n3, n4, and R⁴ are the same as defined with respect to Formula 3 above.

In an embodiment, Formula 3-1 above may be represented by any one selected from among 3-a to 3-d below, Formula 3-2 above may be represented by 3-e below, Formula 3-3 above may be represented by 3-f below, Formula 3-4 above may be represented by 3-g below, and Formula 3-5 above may be represented by 3-h below.

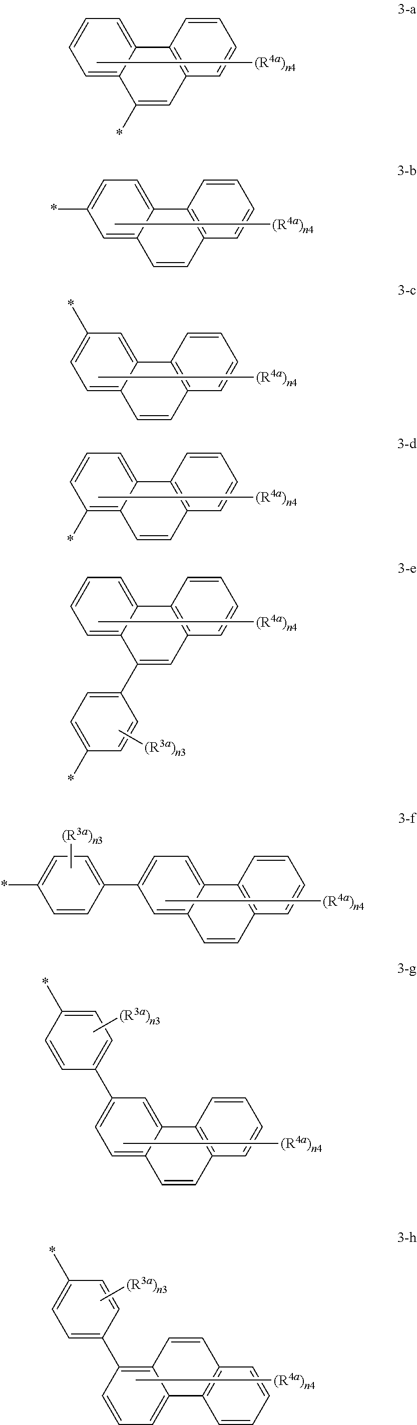

In 3-a to 3-h above, n3, n4, and R^{3a} are the same as defined with respect to Formula 3-1 to Formula 3-5 above, and R^{4a} is a hydrogen atom, a deuterium atom, a halogen atom, or a substituted or unsubstituted alkyl group having 1 to 10 carbon atoms.

In an embodiment, Formula 4 above may be represented by Formula 4-1 or Formula 4-2 below:

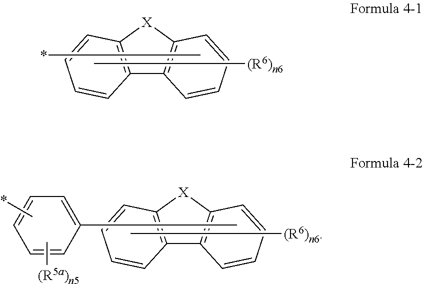

In Formula 4-1 and Formula 4-2 above, R^{5a} is a hydrogen atom, a deuterium atom, or a substituted or unsubstituted phenyl group, X, n5, n6, and R⁶ are the same as defined with respect to Formula 4 above.

In an embodiment, Formula 4-1 above may be represented by 4-a below, and Formula 4-2 may be represented by 4-b or 4-c below:

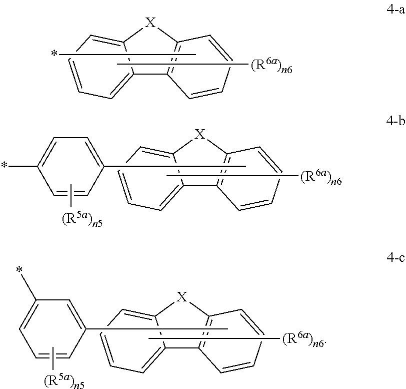

In 4-a to 4-c above, R^{6a} is a hydrogen atom, a deuterium atom, or a substituted or unsubstituted aryl group having 6 to 15 ring-forming carbon atoms, or adjacent R^{6a}'s are bonded to each other to form an aromatic ring, and X, n5, n6, and R^{5a} are the same as defined with respect to Formula 4-1 and Formula 4-2 above.

In an embodiment, Formula 5 above may be represented by any one selected from among Formula 5-1 to Formula 5-4 below:

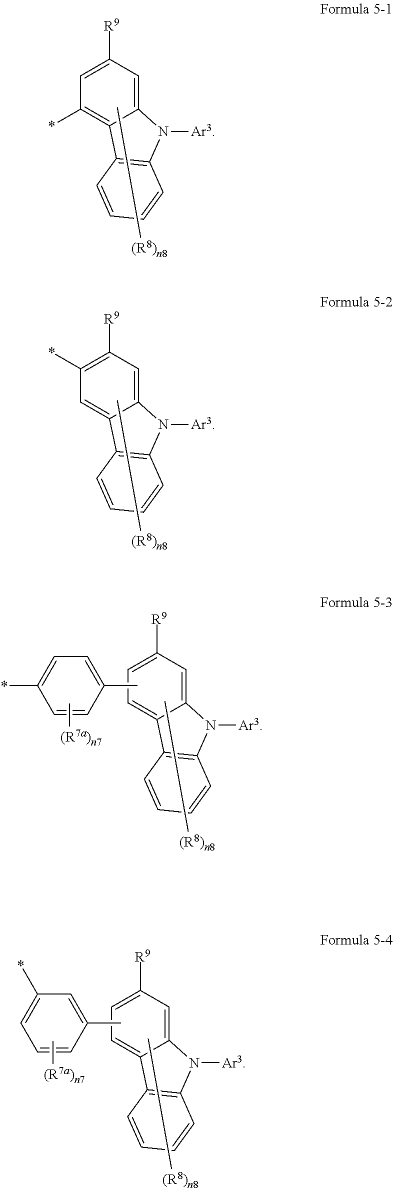

In Formula 5-1 to Formula 5-4 above, R^{7a} is a hydrogen atom or a deuterium atom, and n7, n8, R⁸, R⁹, and Ar³ are the same as defined with respect to Formula 5 above.

In an embodiment, Formula 5-1 above may be represented by 5-a below, Formula 5-2 above may be represented by 5-b below, Formula 5-3 above may be represented by 5-c or 5-d below, and Formula 5-4 above may be represented by 5-e or 5-f below:

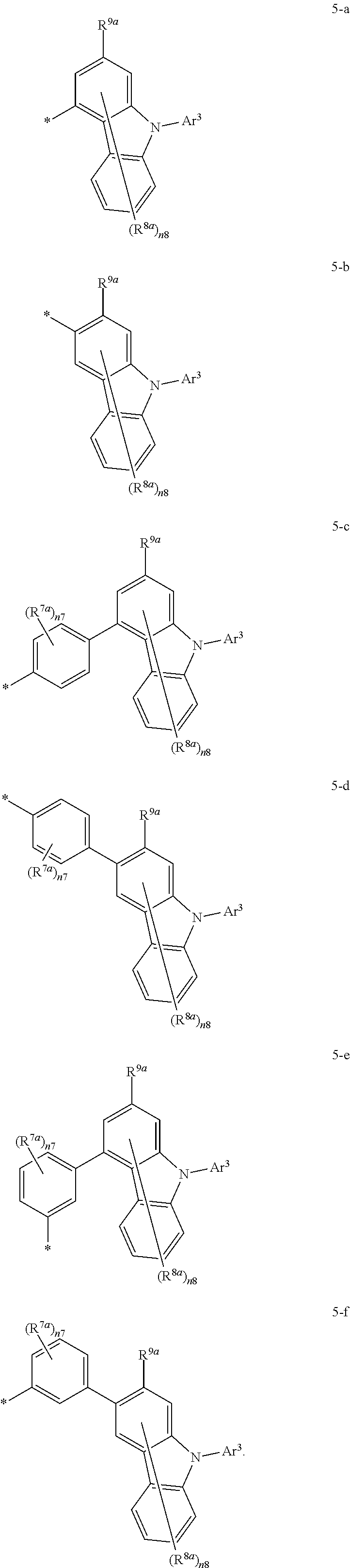

In 5-a to 5-f above, R^{8a} is a hydrogen atom, a deuterium atom, or a substituted or unsubstituted aryl group having 6 to 15 ring-forming carbon atoms, or adjacent R^{8a}'s are bonded to each other to form an aromatic ring, R^{9a} is a hydrogen atom or a deuterium atom, and n7, n8, R^{7a} and Ar³ are the same as defined with respect to Formula 5-1 and Formula 5-4 above.

In an embodiment, R above may be a hydrogen atom, a deuterium atom, a halogen atom, or a substituted or unsubstituted t-butyl group.

In an embodiment of the present disclosure, a light emitting element includes: a first electrode; a second electrode on the first electrode; and at least one functional layer which is between the first electrode and the second electrode and includes the above-described amine compound according to an embodiment.

In an embodiment, the at least one functional layer may include an emission layer, a hole transport region between the first electrode and the emission layer, and an electron transport region between the emission layer and the second electrode, and the hole transport region may include the above-described amine compound according to an embodiment.

In an embodiment, the hole transport region may include at least one of a hole injection layer, a hole transport layer, or an electron blocking layer, and at least one of the hole transport layer or the electron blocking layer may include the described-above amine compound according to an embodiment.

### BRIEF DESCRIPTION OF THE FIGURES

The accompanying drawings are included to provide a further understanding of the subject matter of the present disclosure, and are incorporated in and constitute a part of this specification. The drawings illustrate example embodiments of the present disclosure and, together with the description, serve to explain principles of the subject matter of the present disclosure. In the drawings:

FIG. 1 is a plan view illustrating a display device according to an embodiment of the present disclosure;

FIG. 2 is a cross-sectional view of a display device according to an embodiment of the present disclosure;

FIG. 3 is a cross-sectional view schematically illustrating a light emitting element according to an embodiment of the present disclosure;

FIG. 4 is a cross-sectional view schematically illustrating a light emitting element according to an embodiment of the present disclosure;

FIG. 5 is a cross-sectional view schematically illustrating a light emitting element according to an embodiment of the present disclosure;

FIG. 6 is a cross-sectional view schematically illustrating a light emitting element according to an embodiment of the present disclosure;

FIG. 7 is a cross-sectional view of a display device according to an embodiment of the present disclosure;

FIG. 8 is a cross-sectional view of a display device according to an embodiment of the present disclosure;

FIG. 9 is a cross-sectional view illustrating a display device according to an embodiment of the present disclosure; and

FIG. 10 is a cross-sectional view illustrating a display device according to an embodiment of the present disclosure.

### DETAILED DESCRIPTION

The subject matter of the present disclosure may be modified in many alternate forms, and thus, embodiments will be illustrated in the drawings and described in this text in more detail. It should be understood, however, that it is not intended to limit the present disclosure to the particular forms disclosed, but rather, is intended to cover all modifications, equivalents, and alternatives falling within the spirit and scope of the present disclosure.

When explaining each of the drawings, like reference numerals are used for referring to like elements. In the accompanying drawings, the dimensions of each structure may be exaggerated for clarity of the present disclosure. It will be understood that, although the terms “first”, “second”, etc. may be used herein to describe various components, these components should not be limited by these terms. These terms are only used to distinguish one component from another. For example, a first component could be termed a second component, and, similarly, a second component could be termed a first component, without departing from the scope of the present disclosure. As used herein, the singular forms, “a,” “an,” and “the” are intended to include the plural forms as well, unless the context clearly indicates otherwise.

In the present application, it will be understood that the terms “include,” “have” or the like specify the presence of features, numbers, steps, operations, components, parts, or combinations thereof disclosed in the specification, but do not exclude the possibility of presence or addition of one or more other features, numbers, steps, operations, components, parts, or combinations thereof.

In the present application, when a part such as a layer, a film, a region, or a plate is referred to as being “on” or “above” another part, it can be directly on the other part, or an intervening part may also be present. On the contrary, when a part such as a layer, a film, a region, or a plate is referred to as being “under” or “below” another part, it can be directly under the other part, or an intervening part may also be present. In addition, it will be understood that when a part is referred to as being “on” another part, it can be above the other part, or under the other part as well.

In the present specification, the term “substituted or unsubstituted” may mean substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In addition, each of the substituents described above may be substituted or unsubstituted. For example, a biphenyl group may be interpreted as an aryl group or a phenyl group substituted with a phenyl group.

In the present specification, the phrase “bonded to an adjacent group to form a ring” may indicate that one is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In addition, the rings formed by being bonded to each other may be connected to another ring to form a spiro structure.

In the present specification, the term “adjacent group” may mean a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In addition, two methyl groups in 4,5-dimethylphenanthrene may be interpreted as “adjacent groups” to each other.

In the present specification, examples of the halogen atom may include a fluorine atom, a chlorine atom, a bromine atom, or an iodine atom.

In the specification, the alkyl group may be a linear, branched or cyclic type (e.g., a linear alkyl group, a branched alkyl group, or a cyclic alkyl group). The number of carbons in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-heneicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but embodiments of the present disclosure are not limited thereto.

In the present specification, the term “alkenyl group” means a hydrocarbon group including at least one carbon double bond at a main chain (e.g., in the middle) or at a terminal end (e.g., a terminus) of an alkyl group having 2 or more carbon atoms. The alkenyl group may be a linear chain or a branched chain. The carbon number is not specifically limited, but is 2 to 30, 2 to 20 or 2 to 10. Examples of the alkenyl group include a vinyl group, a 1-butenyl group, a 1-pentenyl group, a 1,3-butadienyl group, a styrenyl group, a styrylvinyl group, etc., without limitation.

In the present specification, the term “alkynyl group” means a hydrocarbon group including at least one carbon triple bond at a main chain (e.g., in the middle) or a terminal end (e.g., a terminus) of an alkyl group having 2 or more carbon atoms. The alkynyl group may be a linear chain or a branched chain. The carbon number is not specifically limited, but is 2 to 30, 2 to 20 or 2 to 10. Examples of the alkynyl group include an ethynyl group, a propynyl group, etc., without limitation.

The term “hydrocarbon ring group,” as used herein, means any suitable functional group or substituent derived from an aliphatic hydrocarbon ring. The hydrocarbon ring group may be a saturated hydrocarbon ring group having 5 to 20 ring-forming carbon atoms.

In the present specification, the term “aryl group” means any suitable functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but embodiments of the present disclosure are not limited thereto.

In the present specification, the fluorenyl group may be substituted, and two substituents may be bonded to each other to form a spiro structure. Examples of cases where the fluorenyl group is substituted are as follows. However, embodiments of the present disclosure are not limited thereto.

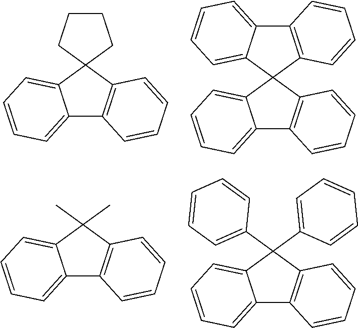

The term “heterocyclic group,” as used herein, means any suitable functional group or substituent derived from a ring including at least one of B, O, N, P, Si, or Se as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the present specification, the heterocyclic group may contain at least one of B, O, N, P, Si, or S as a heteroatom. If the heterocyclic group contains two or more heteroatoms, the two or more heteroatoms may be the same as or different from each other. In the specification, the heterocyclic group may be a monocyclic heterocyclic group or a polycyclic heterocyclic group, and is a concept including a heteroaryl group. The number of ring-forming carbon atoms in the heterocyclic group may be 2 to 30, 2 to 20, or 2 to 10.

In the present specification, the aliphatic heterocyclic group may include one or more among B, O, N, P, Si, and S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but embodiments of the present disclosure are not limited thereto.

The heteroaryl group, as described herein, may include at least one of B, O, N, P, Si, or S as a heteroatom. When the heteroaryl group contains two or more heteroatoms, the two or more heteroatoms may be the same as or different from each other. The heteroaryl group may be a monocyclic heteroaryl group or polycyclic heteroaryl group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a triazole group, a pyridine group, a bipyridine group, a pyrimidine group, a triazine group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but embodiments of the present disclosure are not limited thereto.

In the present specification, the above description of the aryl group may be applied to an arylene group except that the arylene group is a divalent group. The above description of the heteroaryl group may be applied to a heteroarylene group except that the heteroarylene group is a divalent group.

In the present description, the boryl group includes an alkyl boryl group and an aryl boryl group. Examples of the boryl group may include a dimethylboryl group, a diethylboryl group, a t-butylmethylboryl group, a diphenylboryl group, a phenylboryl group, etc., but embodiments of the present disclosure are not limited thereto. For example, the alkyl group in the alkyl boryl group is the same as the examples of the alkyl group described above, and the aryl group in the aryl boryl group is the same as the examples of the aryl group described above.

In the present specification, a silyl group includes an alkyl silyl group and an aryl silyl group. Examples of the silyl group may include trimethylsilyl, triethylsilyl, t-butyldimethylsilyl, vinyldimethylsilyl, propyldimethylsilyl, triphenylsilyl, diphenylsilyl, phenylsilyl, etc. However, embodiments of the present disclosure are not limited thereto.

In the specification, the number of ring-forming carbon atoms in the carbonyl group is not specifically limited, but may be 1 to 40, 1 to 30, or 1 to 20. For example, the carbonyl group may have the following structures, but embodiments of the present disclosure are not limited thereto:

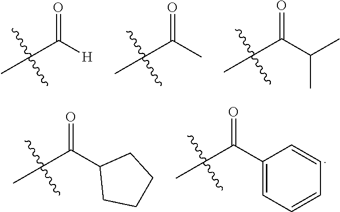

In the present specification, the number of carbon atoms in the sulfinyl group and the sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

In the present specification, a thio group may include an alkylthio group and an arylthio group. The term “thio group,” as used herein, may mean that a sulfur atom is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments of the present disclosure are not limited thereto.

In the present specification, the term “oxy group” may mean that an oxygen atom is bonded to the alkyl group or the aryl group as defined above. The oxy group may include an alkoxy group and an aryl oxy group. The alkoxy group may be a linear chain, a branched chain or a ring chain. The number of carbon atoms in the alkoxy group is not specifically limited, but may be, for example, 1 to 20 or 1 to 10. Examples of the oxy group include methoxy, ethoxy, n-propoxy, isopropoxy, butoxy, pentyloxy, hexyloxy, octyloxy, nonyloxy, decyloxy, benzyloxy, etc., but embodiments of the present disclosure are not limited thereto.

In the present specification, the number of carbon atoms in an amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, etc., but embodiments of the present disclosure are not limited thereto.

In the present specification, the alkyl group among an alkylthio group, an alkylsulfoxy group, an alkylaryl group, an alkylamino group, an alkyl boron group, an alkyl silyl group, and an alkyl amine group is the same as the examples of the alkyl group described above.

In the present specification, the aryl group among an aryloxy group, an arylthio group, an arylsulfoxy group, an arylamino group, an arylboron group, an arylsilyl group, an arylamine group is the same as the examples of the aryl group described above.

In the present specification, a direct linkage may mean a single bond (e.g., a single covalent bond).

As used herein,

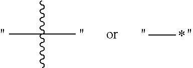

means a position to be connected.

Hereinafter, embodiments of the present disclosure will be described with reference to the accompanying drawings.

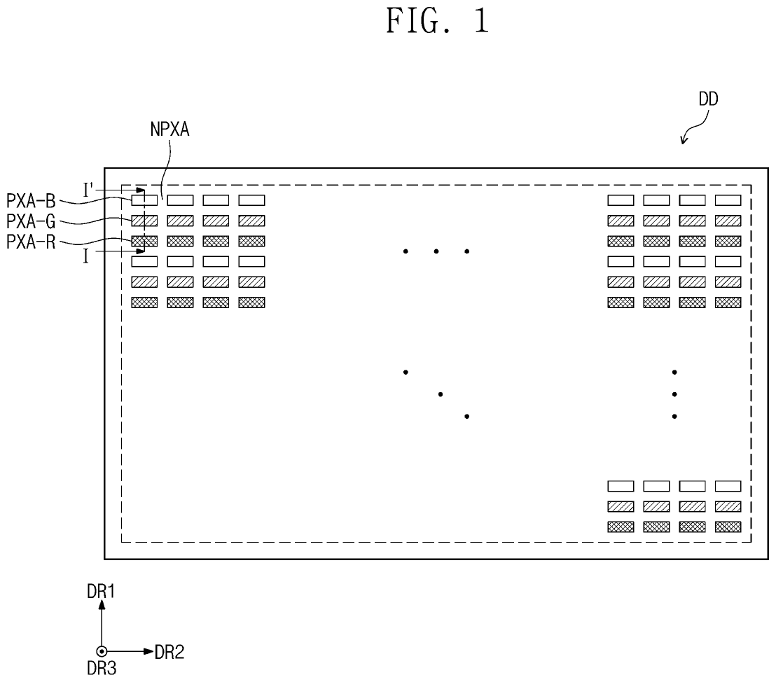

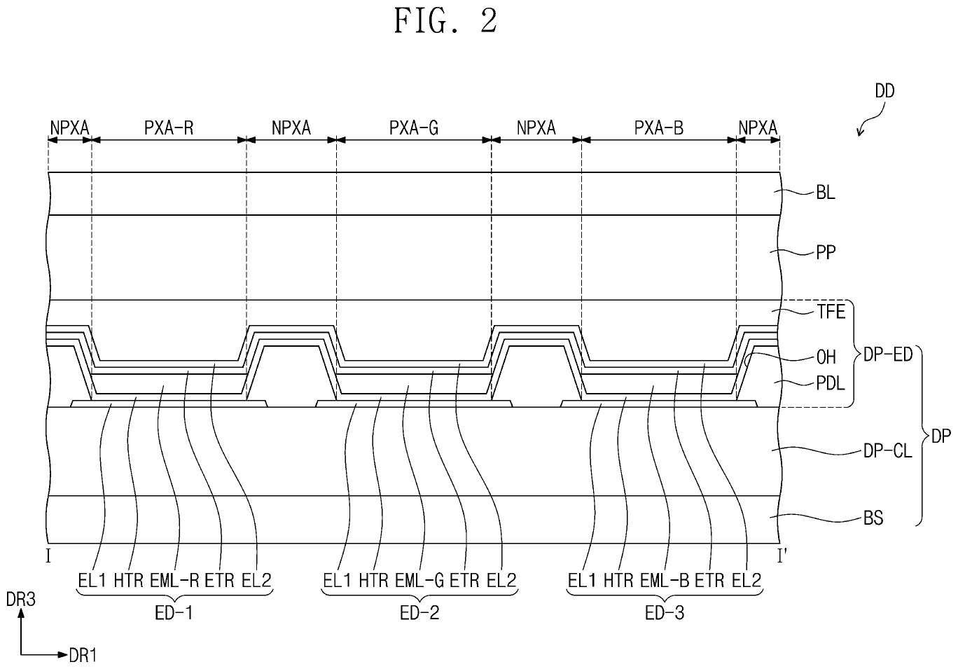

FIG. 1 is a plan view illustrating an embodiment of a display device DD. FIG. 2 is a cross-sectional view of the display device DD of the embodiment. FIG. 2 is a cross-sectional view illustrating a part taken along line I-I′ of FIG. 1.

The display device DD may include a display panel DP and an optical layer PP on the display panel DP. The display panel DP includes light emitting elements ED-1, ED-2, and ED-3. The display device DD may include a plurality of light emitting elements ED-1, ED-2, and ED-3. The optical layer PP may be on the display panel DP to control reflected light in the display panel DP due to external light. The optical layer PP may include, for example, a polarization layer and/or a color filter layer. In some embodiments, the optical layer PP may be omitted from the display device DD of an embodiment.

A base substrate BL may be on the optical layer PP. The base substrate BL may be a member which provides a base surface on which the optical layer PP is located. The base substrate BL may be a glass substrate, a metal substrate, a plastic substrate, etc. However, embodiments of the present disclosure are not limited thereto, and the base substrate BL may be an inorganic layer, an organic layer, or a composite material layer. In some embodiments, the base substrate BL may be omitted.

The display device DD according to an embodiment may further include a filling layer. The filling layer may be between a display element layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display element layer DP-ED. The display element layer DP-ED may include a pixel defining film PDL, the light emitting elements ED-1, ED-2, and ED-3 between portions of the pixel defining film PDL, and an encapsulation layer TFE on the light emitting elements ED-1, ED-2, and ED-3.

The base layer BS may be a member which provides a base surface on which the display element layer DP-ED is located. The base layer BS may be a glass substrate, a metal substrate, a plastic substrate, etc. However, the embodiment is not limited thereto, and the base layer BS may be an inorganic layer, an organic layer, or a composite material layer.

In an embodiment, the circuit layer DP-CL is on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting elements ED-1, ED-2, and ED-3 of the display element layer DP-ED.

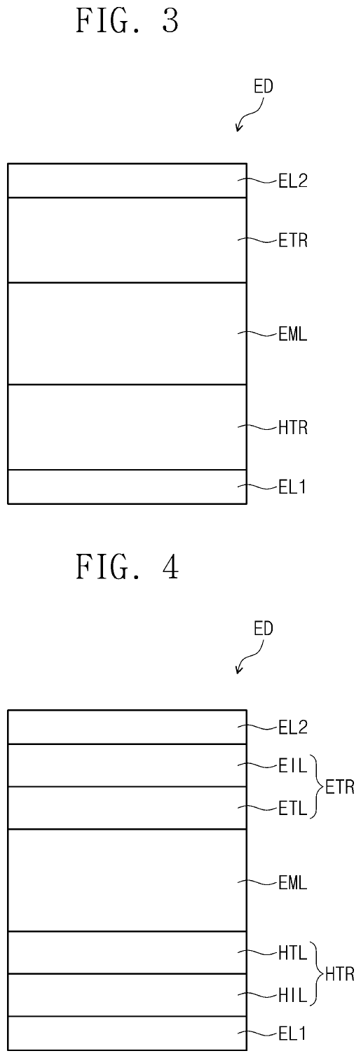

Each of the light emitting elements ED-1, ED-2, and ED-3 may have a structure of a light emitting element ED of an embodiment according to FIGS. 3 to 6, which will be further described herein below. Each of the light emitting elements ED-1, ED-2 and ED-3 may include a first electrode EL1, a hole transport region HTR, emission layers EML-R, EML-G and EML-B, an electron transport region ETR, and a second electrode EL2.

FIG. 2 illustrates an embodiment in which the emission layers EML-R, EML-G, and EML-B of the light emitting elements ED-1, ED-2, and ED-3 are in openings OH defined in the pixel defining film PDL, and the hole transport region HTR, the electron transport region ETR, and the second electrode EL2 are provided as a common layer in the entire light emitting elements ED-1, ED-2, and ED-3. However, embodiments of the present disclosure are not limited thereto, and the hole transport region HTR and the electron transport region ETR in an embodiment may be provided by being patterned inside the openings OH defined in the pixel defining film PDL. For example, the hole transport region HTR, the emission layers EML-R, EML-G, and EML-B, and the electron transport region ETR of the light emitting elements ED-1, ED-2, and ED-3 in an embodiment may be provided by being patterned by an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film protects the display element layer DP-ED from moisture/oxygen, and the encapsulation-organic film protects the display element layer DP-ED from foreign substances such as dust particles. The encapsulation-inorganic film may include silicon nitride, silicon oxynitride, silicon oxide, titanium oxide, aluminum oxide, and/or the like, but embodiments of the present disclosure are not particularly limited thereto. The encapsulation-organic film may include an acrylic-based compound, an epoxy-based compound, and/or the like. The encapsulation-organic film may include a photopolymerizable organic material, but embodiments of the present disclosure are not particularly limited thereto.

The encapsulation layer TFE may be on the second electrode EL2 and may fill the opening OH.

Referring to FIGS. 1 and 2, the display device DD may include a non-light emitting region NPXA and light emitting regions PXA-R, PXA-G and PXA-B. The light emitting regions PXA-R, PXA-G and PXA-B may be regions in which light generated by the respective light emitting elements ED-1, ED-2 and ED-3 is emitted. The light emitting regions PXA-R, PXA-G, and PXA-B may be spaced apart from each other on a plane.

Each of the light emitting regions PXA-R, PXA-G, and PXA-B may be a region divided by the pixel defining film PDL. The non-light emitting regions NPXA may be regions between the adjacent light emitting regions PXA-R, PXA-G, and PXA-B, which correspond to portions of the pixel defining film PDL. In the present specification, the light emitting regions PXA-R, PXA-G, and PXA-B may respectively correspond to pixels. The pixel defining film PDL may divide the light emitting elements ED-1, ED-2, and ED-3. The emission layers EML-R, EML-G and EML-B of the light emitting elements ED-1, ED-2 and ED-3 may be in openings OH defined in the pixel defining film PDL and separated from (spaced apart from) each other.

The light emitting regions PXA-R, PXA-G and PXA-B may be divided into a plurality of groups according to the color of light generated from the light emitting elements ED-1, ED-2 and ED-3. In the display device DD of an embodiment shown in FIGS. 1 and 2, three light emitting regions PXA-R, PXA-G, and PXA-B which emit red light, green light, and blue light, respectively are illustrated as examples. For example, the display device DD of an embodiment may include the red light emitting region PXA-R, the green light emitting region PXA-G, and the blue light emitting region PXA-B that are separated from (spaced apart from) each other.

In the display device DD according to an embodiment, the plurality of light emitting elements ED-1, ED-2 and ED-3 may emit light beams having wavelengths different from each other. For example, in an embodiment, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, embodiments of the present disclosure are not limited thereto, and the first to third light emitting elements ED-1, ED-2, and ED-3 may emit light beams in the same wavelength range or at least one light emitting element may emit a light beam in a wavelength range different from the others. For example, the first to third light emitting elements ED-1, ED-2, and ED-3 may all emit blue light.

The light emitting regions PXA-R, PXA-G, and PXA-B in the display device DD according to an embodiment may be in a stripe form. Referring to FIG. 1, the plurality of red light emitting regions PXA-R, the plurality of green light emitting regions PXA-G, and the plurality of blue light emitting regions PXA-B each may be arranged along a second directional axis DR2. In some embodiments, the red light emitting region PXA-R, the green light emitting region PXA-G, and the blue light emitting region PXA-B may be alternately arranged in this order along a first directional axis DR1.

FIGS. 1 and 2 illustrate that all the light emitting regions PXA-R, PXA-G, and PXA-B have similar area, but embodiments of the present disclosure are not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may mean areas when viewed on a plane defined by the first directional axis DR1 and the second directional axis DR2.

In some embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in various suitable combinations according to the characteristics of display quality required or desired in the display device DD. For example, the arrangement form of the light emitting regions PXA-R, PXA-G, and PXA-B may be a PENTILE® arrangement form (e.g., an RGBG matrix, RGBG structure, or RGBG matrix structure) or a Diamond Pixel™ arrangement form. PENTILE® is a duly registered trademark of Samsung Display Co., Ltd.

In some embodiments, the areas of the light emitting regions PXA-R, PXA-G, and PXA-B may be different from each other. For example, in an embodiment, the area of the green light emitting region PXA-G may be smaller than that of the blue light emitting region PXA-B, but embodiments of the present disclosure are not limited thereto.

Hereinafter, FIGS. 3 to 6 are cross-sectional views schematically illustrating light emitting elements according to embodiments. The light emitting elements ED according to embodiments each may include a first electrode EL1, a second electrode EL2 facing the first electrode EL1, and at least one functional layer between the first electrode EL1 and the second electrode EL2. Each of the light emitting elements ED of embodiments may include an amine compound of an embodiment, which will be further described below, in at least one functional layer.

Each of the light emitting elements ED may include, as at least one functional layer, a hole transport region HTR, an emission layer EML, and an electron transport region ETR that are sequentially stacked. Referring to FIG. 3, the light emitting element ED of an embodiment may include a first electrode EL1, a hole transport region HTR, an emission layer EML, an electron transport region ETR, and a second electrode EL2 that are sequentially stacked.

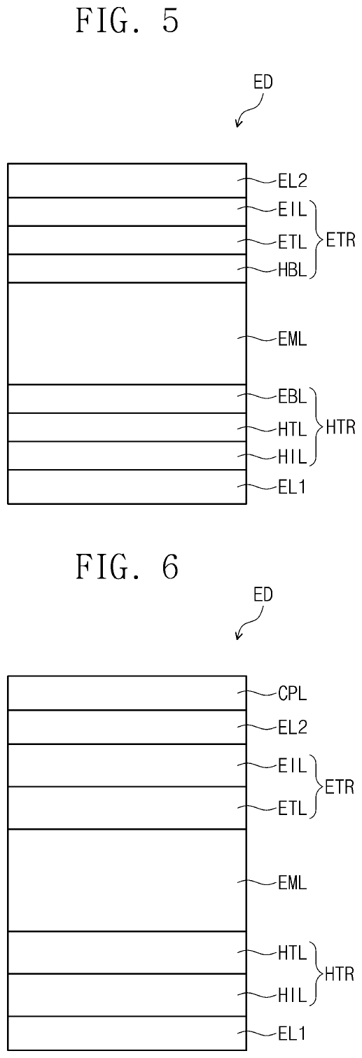

Compared with FIG. 3, FIG. 4 illustrates a cross-sectional view of a light emitting element ED of an embodiment, in which a hole transport region HTR includes a hole injection layer HIL and a hole transport layer HTL, and an electron transport region ETR includes an electron injection layer EIL and an electron transport layer ETL. Compared with FIG. 3, FIG. 5 illustrates a cross-sectional view of a light emitting element ED of an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting element ED of an embodiment including a capping layer CPL on a second electrode EL2.

The light emitting element ED of an embodiment may include an amine compound of an embodiment, which will be further described below, in the hole transport region HTR. In the light emitting element ED of an embodiment, at least one of a hole injection layer HIL, a hole transport layer HTL, or an electron blocking layer EBL in the hole transport region HTR may include the amine compound of an embodiment. For example, in the light emitting element ED of an embodiment, at least one of the hole transport layer HTL or the electron blocking layer EBL may include the amine compound of an embodiment.

In the light emitting element ED according to an embodiment, the first electrode EL1 has conductivity (e.g., electrical conductivity). The first electrode EL1 may be formed of a metal material, a metal alloy, or a conductive compound. The first electrode EL1 may be an anode or a cathode. However, embodiments of the present disclosure are not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, Zn, a compound of two or more thereof, a mixture of two or more thereof, and an oxide thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, and/or a compound or mixture thereof (e.g., a mixture of Ag and Mg). In some embodiments, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but embodiments of the present disclosure are not limited thereto. Embodiments of the present disclosure are not limited thereto, however, and the first electrode EL1 may include the above-described metal materials, combinations of at least two metal materials of the above-described metal materials, oxides of the above-described metal materials, and/or the like. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may have a single layer formed of a single material, a single layer formed of a plurality of different materials, or a multilayer structure including a plurality of layers formed of a plurality of different materials.

The hole transport region HTR may include at least one of the hole injection layer HIL, the hole transport layer HTL, or the electron blocking layer EBL. In some embodiments, the hole transport region HTR may include a plurality of stacked hole transport layers.

In some embodiments, the hole transport region HTR may have a single layer structure of the hole injection layer HIL or the hole transport layer HTL, or may have a single layer structure formed of a hole injection material and a hole transport material. In an embodiment, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, or a hole transport layer HTL/buffer layer are stacked in order from the first electrode EL1, but embodiments of the present disclosure are not limited thereto.

The thickness of the hole transport region HTR may be, for example, from about 50 Å to about 15,000 Å. The hole transport region HTR may be formed using various suitable methods such as a vacuum deposition method, a spin coating method, a cast method, a Langmuir-Blodgett (LB) method, an inkjet printing method, a laser printing method, and/or a laser induced thermal imaging (LITI) method.

The light emitting element ED of an embodiment may include the amine compound of an embodiment in the hole transport region HTR. In the light emitting element ED of an embodiment, the hole transport layer HTL or the electron blocking layer EBL in the hole transport region HTR may include the amine compound of an embodiment. The amine compound of an embodiment may include a phenanthrene moiety directly linked to the nitrogen atom. In some embodiments, referring to Formula a below, for the phenanthrene moiety in the amine compound of an embodiment, the nitrogen atom may be directly linked at the third position of the phenanthrenyl group.

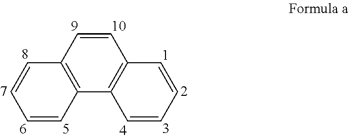 [SMILES: c1ccc2c(c1)ccc1ccccc12]

In an embodiment according to the present disclosure, the amine compound may be represented by Formula 1 below.

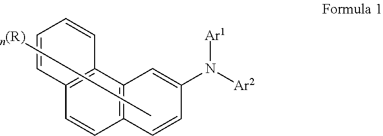

In Formula 1, n may be an integer of 0 to 9. R may be a hydrogen atom, a deuterium atom, a halogen atom, or a substituted or unsubstituted alkyl group having 1 to 20 carbon atoms. For example, R may be a hydrogen atom, a deuterium atom, or a substituted or unsubstituted t-butyl group. In some embodiments, when R is a halogen atom, R may include a fluorine atom (F) as a heteroatom.

In an embodiment, when n is an integer of 2 or more, a plurality of R's may all be the same or at least one may be different from the rest. In some embodiments, when n is 0, the phenanthrene moiety may be unsubstituted with R.

In Formula 1, Ar¹ and Ar² may be each independently represented by any one selected from among Formula 2 to Formula 5 below. The amine compound represented by Formula 1 of an embodiment may have improved electron resistance and exciton resistance of the material by introducing two substituents among the substituents represented by Formula 2 to Formula 5 below into the amine moiety.

In some embodiments, the amine compound represented by Formula 1 of an embodiment may include a structure in which any hydrogen atom in the molecule is substituted with a deuterium atom. For example, at least one of R, Ar₁, or Ar₂ in Formula 1 may include a deuterium atom, or a substituent including a deuterium atom. For example, the amine compound of an embodiment may include at least one deuterium atom as a substituent. In some embodiments, the amine compound of an embodiment may be a monoamine compound. The amine compound of an embodiment may not include an amine group as a substituent.

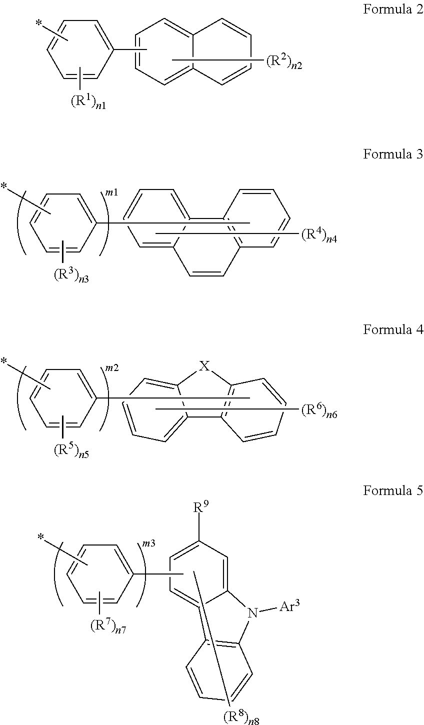

In Formula 2 to Formula 5, [symbol] may be a part linked to the nitrogen atom in Formula 1 above. R¹ to R⁵, R⁷, and R⁹ may be each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms. In an embodiment, R¹ to R⁵, R⁷, and R⁹ may be each independently a hydrogen atom, a deuterium atom, a halogen atom, a substituted or unsubstituted alkyl group having 1 to 20 carbon atoms, or a substituted or unsubstituted aryl group having 6 to 30 ring-forming carbon atoms. For example, R¹, R³, R⁵, and R⁷ may be each independently a hydrogen atom, or a substituted or unsubstituted aryl group having 6 to 15 ring-forming carbon atoms. R² may be a hydrogen atom, a deuterium atom, or a substituted or unsubstituted aryl group having 6 to 15 ring-forming carbon atoms. R⁴ may be a hydrogen atom, a deuterium atom, a halogen atom, or a substituted or unsubstituted alkyl group having 1 to 10 carbon atoms, and R⁹ may be a hydrogen atom or a deuterium atom. However, embodiments of the present disclosure are not limited thereto. In some embodiments, when R¹ to R⁹ are halogen atoms, R¹ to R⁹ each may include a fluorine atom (F) as a heteroatom.

In Formula 4 and Formula 5, R⁶ and R⁸ may be each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, or may be bonded to an adjacent group to form a ring. When R⁶ and R⁸ are bonded to an adjacent group to form a ring, for each of R⁶ and R⁸, adjacent R⁶'s may be bonded to each other to form an aromatic ring, and adjacent R⁸'s may be bonded to each other to form an aromatic ring. For example, R⁶ and R⁸ may be each independently a hydrogen atom, a deuterium atom, a substituted or unsubstituted aryl group having 6 to 15 ring-forming carbon atoms, or adjacent R⁶'s or adjacent R⁸'s may be bonded to each other to form an aromatic ring. When adjacent R⁶'s are bonded to each other to form an aromatic ring, the amine compound of an embodiment may include a benzonaphthofuran moiety or a benzonaphthothiophene moiety. In some embodiments, when adjacent R⁸'s are bonded to each other to form an aromatic ring, the amine compound of an embodiment may include a benzocarbazole moiety.

In Formula 2 to Formula 5, n1, n3, n5, and n7 means the number of R¹, R³, R⁵, and R⁷, respectively. n1, n3, n5, and n7 may be each independently an integer of 0 to 4. n2, and n6 may be each independently an integer of 0 to 7, n4 may be an integer of 0 to 9, and n8 may be an integer of 0 to 6. In an embodiment, the case where each of n1, n3, n5, and n7 is 0 may mean that the linker of a substituted or unsubstituted phenyl group is not substituted with each of R¹, R³, R⁵, and R⁷. In some embodiments, the case where each of n2, n4, n6, and n8 is 0 may mean that a naphthylene moiety in Formula 2 is not substituted with R², a phenanthrene moiety in Formula 3 is not substituted with R⁴, a dibenzoheterole moiety in Formula 4 is not substituted with R⁶, and a carbazole moiety in Formula 5 is not substituted with R⁸.

In an embodiment, when each of n1 to n8 is an integer of 2 or greater, a plurality of R₁'s to R₈'s may each be the same or at least one may be different from the rest. For example, when n1 is 2, two R¹'s may be the same as or different from each other. In some embodiments, the foregoing description may be equally applied to R², R³, R⁴, R⁵, R⁶, R⁷, and R⁸.

In Formula 3 to Formula 5, m1 to m3 may be each independently 0 or 1. When each of m1 to m3 is 0, each of the phenanthrene moiety in Formula 3, the dibenzoheterole moiety in Formula 4, and the carbazole moiety in formula 5 may be directly linked to the nitrogen atom in Formula 1. When each of m1 to m3 is 1, each of the phenanthrene moiety in Formula 3, the dibenzoheterole moiety in Formula 4, and the carbazole moiety in formula 5 may be linked to the amine compound of an embodiment via a linker of a substituted or unsubstituted phenylene group.

In Formula 4, X may be O or S. For example, when X is O, the dibenzofuran moiety may be substituted with at least one R⁶ or unsubstituted. When X is S, the dibenzothiophene moiety may be a dibenzothiophene group substituted with at least one R⁶ or unsubstituted. In Formula 5, Ar³ may be a substituted or unsubstituted phenyl group. For example, Ar³ may be a t-butyl group-substituted or unsubstituted phenyl group. However, embodiments of the present disclosure are not limited thereto.

In some embodiments, in the amine compound represented by Formula 1 of an embodiment, when Ar¹ and Ar² are each independently represented by any one selected from among Formula 3 to Formula 5 above, at least one selected from among m1 to m3 may be 1. For example, when Ar¹ is represented by Formula 3 and Ar² is represented by Formula 4 or Formula 5, for the amine compound of an embodiment, at least one of Ar¹ or Ar² linked to the nitrogen atom may have a linker such as a substituted or unsubstituted phenylene group. In some embodiments, the foregoing description may be equally applied to the case where Ar¹ is represented by Formula 4 and Ar² is represented by Formula 4 or Formula 5, or Ar¹ is represented by Formula 5 and Ar² is represented by Formula 5. In the amine compound represented by Formula 1 of an embodiment, the case where both Ar¹ and Ar² are represented by Formula 3 above may be excluded. In some embodiments, in the amine compound represented by Formula 1 of an embodiment, when both Ar¹ and Ar² are represented by Formula 4 above, any one selected from among two m2's may be 1, and the other may be 0.

In an embodiment, Formula 2 may be represented by Formula 2-1 or Formula 2-2 below. Each of Formula 2-1 and Formula 2-2 corresponds to the case where the linkage position of the naphthyl group linked to a linker is different.

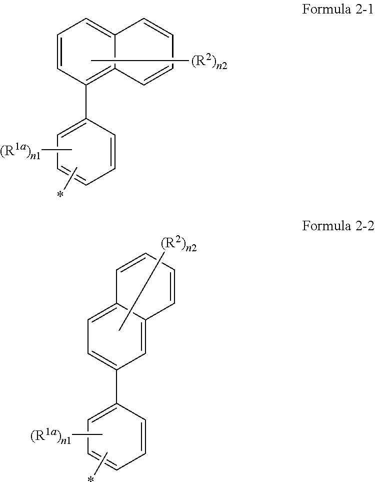

In Formula 2-1 and Formula 2-2, R^{1a} may be a hydrogen atom, a deuterium atom, or a substituted or unsubstituted phenyl group. The same as described with respect to Formula 2 above may be applied to n1, n2, and R².

In an embodiment, Formula 2-1 and Formula 2-2 may be represented by any one selected from among 2-a to 2-d below. For example, Formula 2-1 may be represented by 2-a or 2-b below, and Formula 2-2 may be represented by 2-c or 2-d below. In an embodiment, 2-a below corresponds to the case where the naphthyl group in Formula 2-1 is linked to the nitrogen atom in Formula 1 in the para-relation (e.g., at the para position), and 2-b corresponds to the case where the naphthyl group in Formula 2-1 is linked to the nitrogen atom of the amine compound in the meta-relation (e.g., at the meta position). In some embodiments, 2-c corresponds to the case where the naphthyl group in Formula 2-2 is linked to the nitrogen atom of the amine compound in the para-relation (e.g., at the para position), and 2-d corresponds to the case where the naphthyl group in Formula 2-2 is linked to the nitrogen atom of Formula 1 in the meta-relation (e.g., at the meta position).

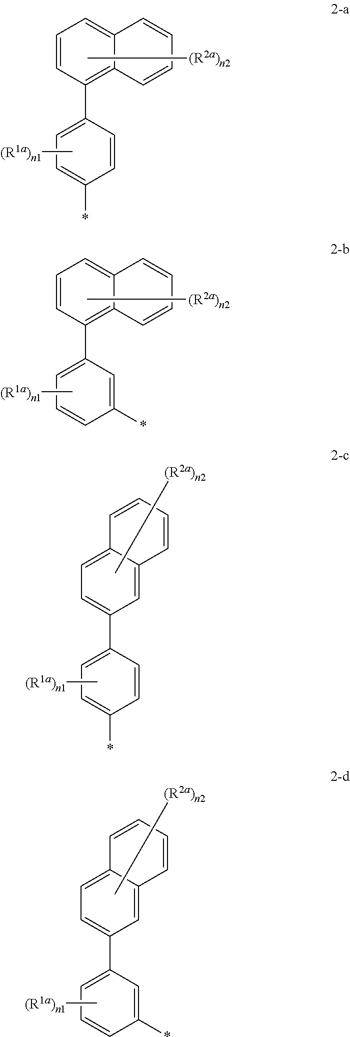

In 2-a to 2-d above, R^{2a} may be a hydrogen atom, a deuterium atom, or a substituted or unsubstituted aryl group having 6 to 15 ring-forming carbon atoms. For example, R^{2a} may be a hydrogen atom, a deuterium atom, or a substituted or unsubstituted phenyl group. The same as described with respect to Formula 2-1 and Formula 2-2 above may be applied to n1, n2, and R^{1a}.

In an embodiment, Formula 3 may be represented by any one selected from among Formula 3-1 to Formula 3-5. Formula 3-1 corresponds to the case where the phenanthryl group is directly linked to the nitrogen atom of the amine compound, and Formula 3-2 to Formula 3-5 correspond to the cases where the phenanthryl group is linked to the nitrogen atom of the amine compound via a linker of a substituted or unsubstituted phenylene group. In some embodiments, each of Formula 3-2 to Formula 3-5 may correspond to the case where the linkage position of the naphthyl group linked to a linker is different.

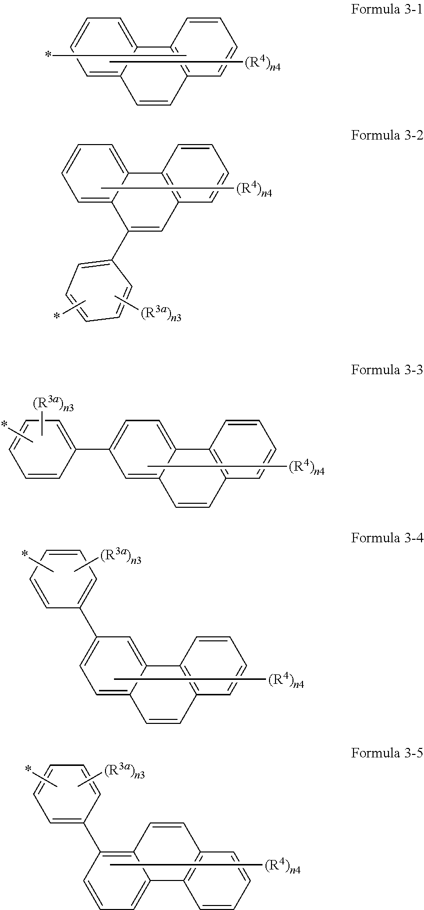

In Formula 3-1 to Formula 3-5, R^{3a} may be a hydrogen atom, a deuterium atom, or a substituted or unsubstituted phenyl group. The same as described with respect to Formula 3 above may be applied to n3, n4, and R⁴.

In an embodiment, Formula 3-1 to Formula 3-5 may be represented by any one selected from among 3-a to 3-h below. For example, Formula 3-1 may be represented by any one selected from among 3-a to 3-d below, Formula 3-2 may be represented by 3-e below, Formula 3-3 may be represented by 3-f below, Formula 3-4 may be represented by 3-g below, and Formula 3-5 may be represented by 3-h below. 3-a to 3-d below illustrate the cases where the phenanthryl group in Formula 3-1 is directly linked to the nitrogen atom of the amine compound, and 3-e to 3-h illustrate the cases where the phenanthryl group is linked to the nitrogen atom of the amine compound via a linker of a substituted or unsubstituted phenylene group. In some embodiments, 3-e to 3-h correspond to the cases where the phenanthryl group in Formula 3-2 to Formula 3-5 is linked to the nitrogen atom of the amine compound in the para-relation (e.g., at the para position).

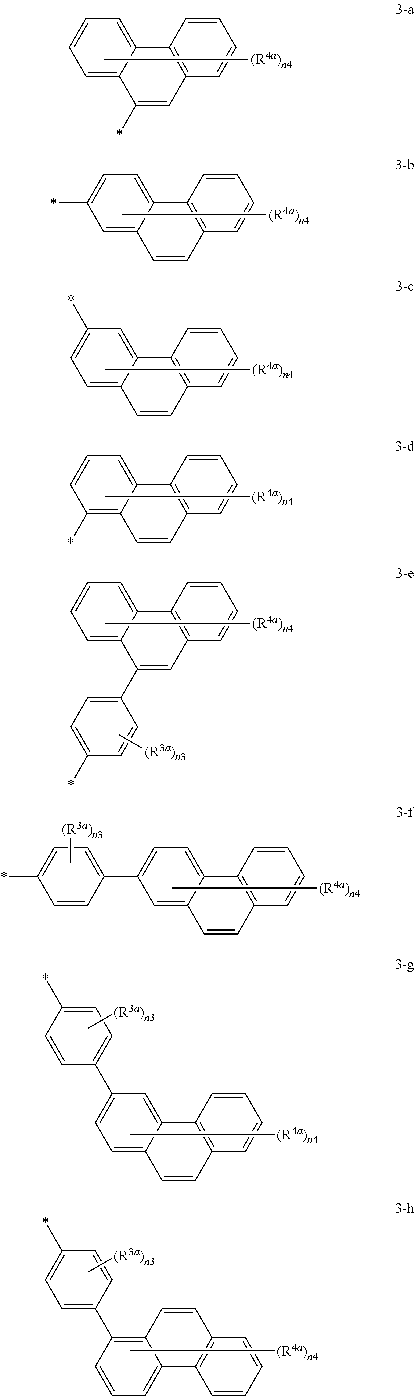

In 3-a to 3-h above, R^{4a} may be a hydrogen atom, a deuterium atom, a halogen atom, or a substituted or unsubstituted alkyl group having 1 to 10 carbon atoms. For example, R^{4a} may be a hydrogen atom, a deuterium atom, a halogen atom, or a substituted or unsubstituted t-butyl group. The same as described with respect to Formula 3-1 to Formula 3-5 above may be applied to n3, n4, and R^{3a}. In some embodiments, when R^{4a} is a halogen atom, R^{4a} may include a fluorine atom (F) as a heteroatom.

In an embodiment, Formula 4 may be represented by Formula 4-1 or Formula 4-2. Formula 4-1 corresponds to the case where the dibenzoheterole group is directly linked to the nitrogen atom of the amine compound, and Formula 4-2 corresponds to the case where the dibenzoheterole group is linked to the nitrogen atom of the amine compound via a linker of a substituted or unsubstituted phenylene group.

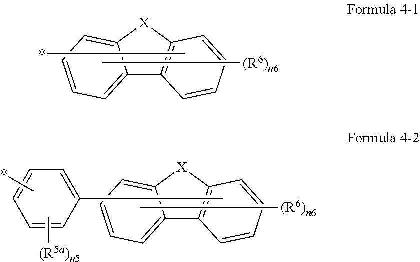

In Formula 4-1 and Formula 4-2, R^{5a} may be a hydrogen atom, a deuterium atom, or a substituted or unsubstituted phenyl group. The same as described with respect to Formula 4 above may be applied to n5, n6, X, and R⁶.

In an embodiment, Formula 4-1 and Formula 4-2 may be represented by any one selected from among 4-a to 4-c below. For example, Formula 4-1 may be represented by 4-a below, and Formula 4-2 may be represented by 4-b or 4-c below. 4-a corresponds to the case where the dibenzoheterole group in Formula 4-1 is directly linked to the nitrogen atom of the amine compound, and R⁶ is specified. 4-b corresponds to the case where the dibenzoheterole group in Formula 4-2 is linked to the nitrogen atom of the amine compound in the para-relation (e.g., at the para position), and 4-c corresponds to the case where the dibenzoheterole group in Formula 4-2 is linked to the nitrogen atom of the amine compound in the meta-relation (e.g., at the meta position). In some embodiments, 4-b and 4-c correspond to the cases where R⁶ in Formula 4-2 is specified.

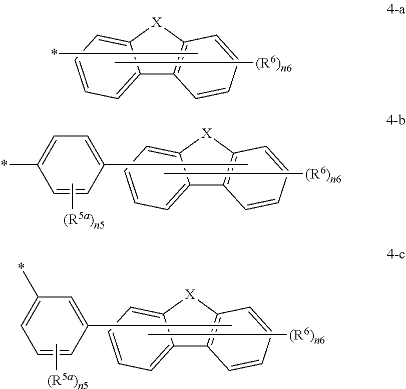

The same as described with respect to Formula 4-1 and Formula 4-2 above may be applied to X, n5, n6, and R^{5a} in 4-a to 4-c above. In an embodiment, R^{6a} may be a hydrogen atom, a deuterium atom, or a substituted or unsubstituted aryl group having 6 to 15 ring-forming carbon atoms, or adjacent R^{6a}'s may be bonded to each other to form an aromatic ring. For example, R^{6a} may be a hydrogen atom, a deuterium atom, or a substituted or unsubstituted phenyl group. In some embodiments, for R^{6a}, adjacent R^{6a}'s may be bonded to each other to form an aromatic ring, and in this case, 4-a to 4-c above may include a benzonaphthofuran skeleton or a benzonaphthothiophene skeleton.

In an embodiment, Formula 5 may be represented by any one selected from among Formula 5-1 to Formula 5-4 below. Formula 5-1 and Formula 5-2 correspond to the cases where the carbazole group is directly linked to the nitrogen atom of the amine compound, and Formula 5-3 and Formula 5-4 correspond to the cases where the carbazole group is linked to the nitrogen atom of the amine compound via a linker of a substituted or unsubstituted phenylene group. In some embodiments, Formula 5-1 may correspond to the case where the nitrogen atom in the amine compound represented by Formula 1 is located in the meta-relation to the nitrogen atom of the carbazole group (e.g., is located at the meta position). Formula 5-2 may correspond to the case where the nitrogen atom in the amine compound represented by Formula 1 is located in the para-relation to the nitrogen atom of the carbazole group (e.g., at the para position). In some embodiments, Formula 5-3 may correspond to the case where the carbazole group is linked to the nitrogen atom in the amine compound via a linker at the para-position (e.g., at the para position), and Formula 5-4 may correspond to the case where the carbazole group is linked to the nitrogen atom in the amine compound via a linker at the meta-position.

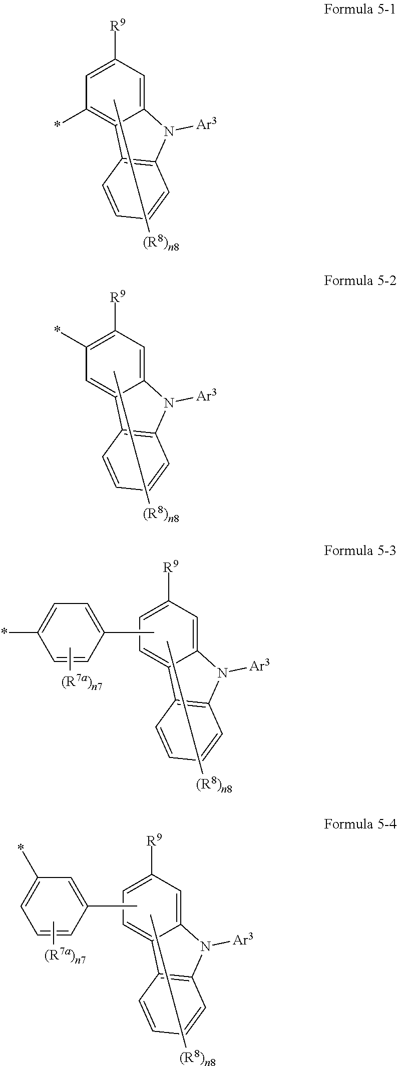

In Formula 5-1 to Formula 5-4, R^{7a} may be a hydrogen atom or a deuterium atom. The same as described with respect to Formula 5 above may be applied to n7, n8, R⁸, and R⁹.

In an embodiment, Formula 5-1 to Formula 5-4 may be represented by any one selected from among 5-a to 5-f below. For example, Formula 5-1 may be represented by 5-a below, Formula 5-2 may be represented by 5-b below, Formula 5-3 may be represented by 5-c or 5-d below, and Formula 5-4 may be represented by 5-e or 5-f below. 5-a and 5-b are the cases where the carbazole group is directly linked to the nitrogen atom of the amine compound in Formula 5-1 and Formula 5-2, respectively, and correspond the cases where R⁸ and R⁹ in Formula 5-1 and Formula 5-2 are specified, respectively. 5-c to 5-f are the cases where the carbazole group in Formula 5-3 and Formula 5-4 is linked to the nitrogen atom of the amine compound via a linker of a substituted or unsubstituted phenylene group, and R⁸ and R⁹ are specified. In some embodiments, 5-c to 5-f correspond to the cases where the carbazole group in Formula 5-3 and Formula 5-4 is linked to the nitrogen atom of the amine compound in the meta-relation (e.g., at the meta position) or para-relation (e.g., at the para position).

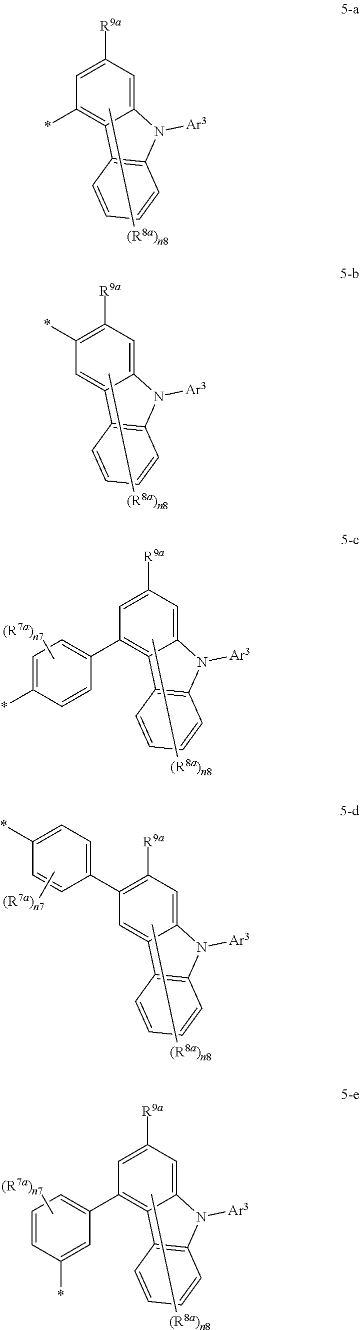

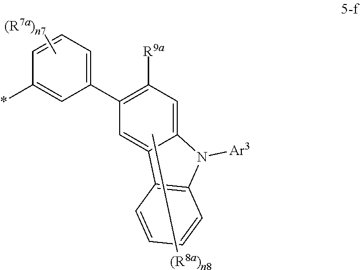

In 5-a to 5-f above, R^{8a} may be a hydrogen atom, a deuterium atom, a substituted or unsubstituted aryl group having 6 to 15 ring-forming carbon atoms, or adjacent R^{8a}'s may be bonded to each other to form an aromatic ring. For example, R^{8a} may be a hydrogen atom, a deuterium atom, or a substituted or unsubstituted phenyl group. In some embodiments, for R^{8a}, adjacent R^{8a}'s may be bonded to each other to form an aromatic ring, and in this case, 5-a to 5-f above may include a benzocarbazole skeleton.

In 5-a to 5-f above, R^{9a} may be a hydrogen atom or a deuterium atom. The same as described with respect to Formula 5-1 to Formula 5-4 above may be applied to n7, n8, R^{7a}, and Ar³.

The amine compound represented by Formula 1 of an embodiment may be represented by one selected from among the compounds of Compound Group 1 below. The hole transport region HTR of the light emitting element ED of an embodiment may include at least one selected from among the amine compounds disclosed in Compound Group 1 below. D in Compound Group 1 below is a deuterium atom.

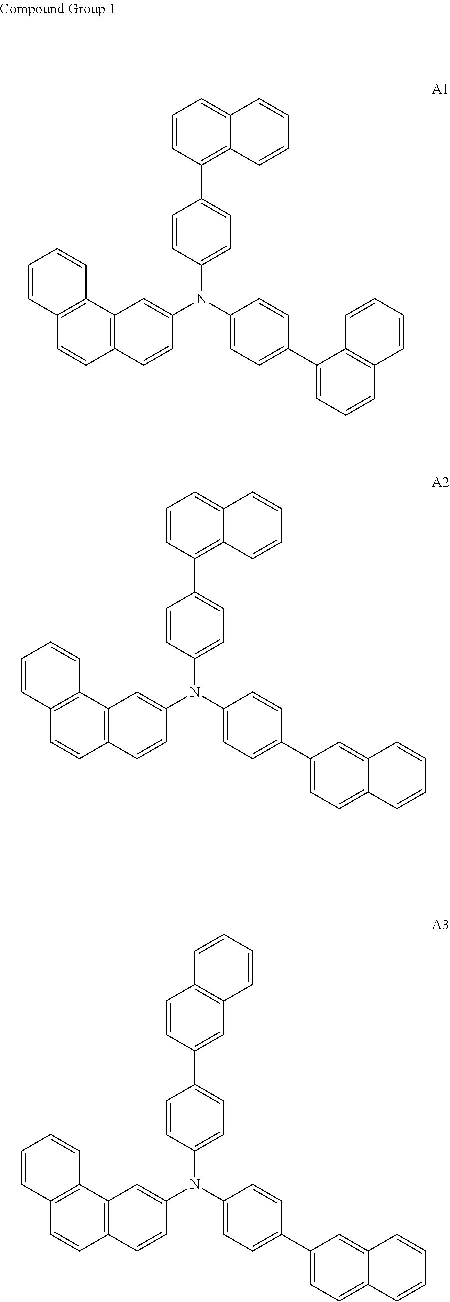 [SMILES: c1ccc2c(-c3ccc(N(c4ccc(-c5cccc6ccccc56)cc4)c4ccc5ccc6ccccc6c5c4)cc3)cccc2c1 ; c1ccc2cc(-c3ccc(N(c4ccc(-c5cccc6ccccc56)cc4)c4ccc5ccc6ccccc6c5c4)cc3)ccc2c1 ; c1ccc2cc(-c3ccc(N(c4ccc(-c5ccc6ccccc6c5)cc4)c4ccc5ccc6ccccc6c5c4)cc3)ccc2c1]

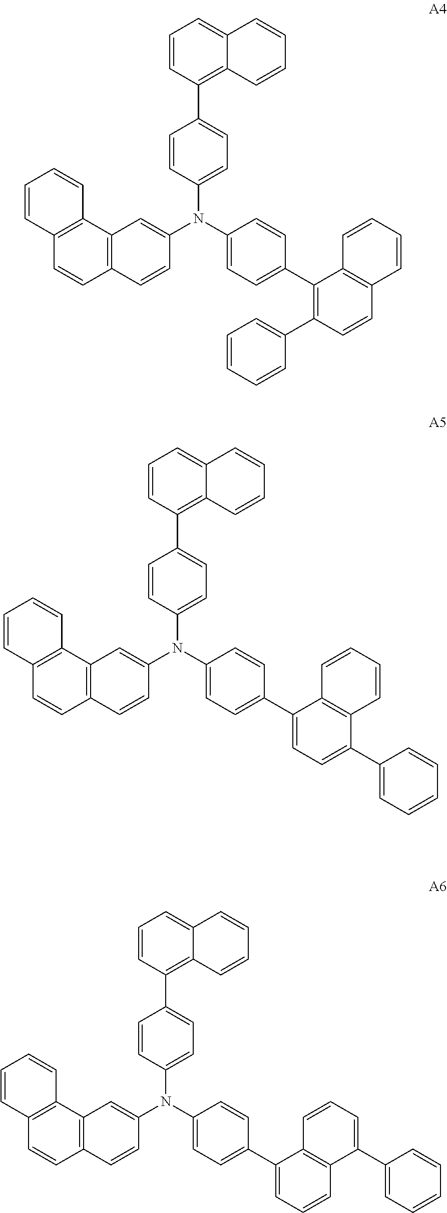 [SMILES: c1ccc(-c2ccc3ccccc3c2-c2ccc(N(c3ccc(-c4cccc5ccccc45)cc3)c3ccc4ccc5ccccc5c4c3)cc2)cc1 ; c1ccc(-c2ccc(-c3ccc(N(c4ccc(-c5cccc6ccccc56)cc4)c4ccc5ccc6ccccc6c5c4)cc3)c3ccccc23)cc1 ; c1ccc(-c2cccc3c(-c4ccc(N(c5ccc(-c6cccc7ccccc67)cc5)c5ccc6ccc7ccccc7c6c5)cc4)cccc23)cc1]

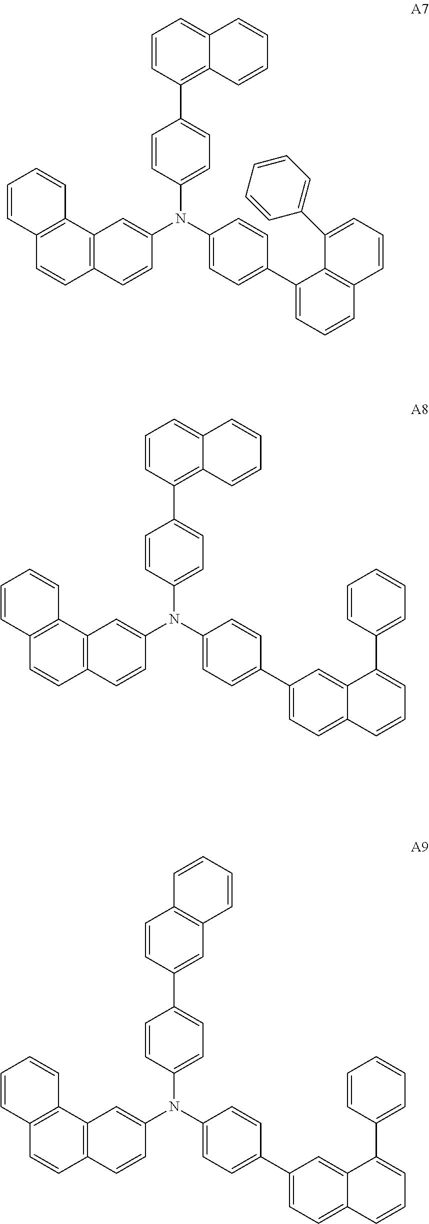 [SMILES: c1ccc(-c2cccc3cccc(-c4ccc(N(c5ccc(-c6cccc7ccccc67)cc5)c5ccc6ccc7ccccc7c6c5)cc4)c23)cc1 ; c1ccc(-c2cccc3ccc(-c4ccc(N(c5ccc(-c6cccc7ccccc67)cc5)c5ccc6ccc7ccccc7c6c5)cc4)cc23)cc1 ; c1ccc(-c2cccc3ccc(-c4ccc(N(c5ccc(-c6ccc7ccccc7c6)cc5)c5ccc6ccc7ccccc7c6c5)cc4)cc23)cc1]

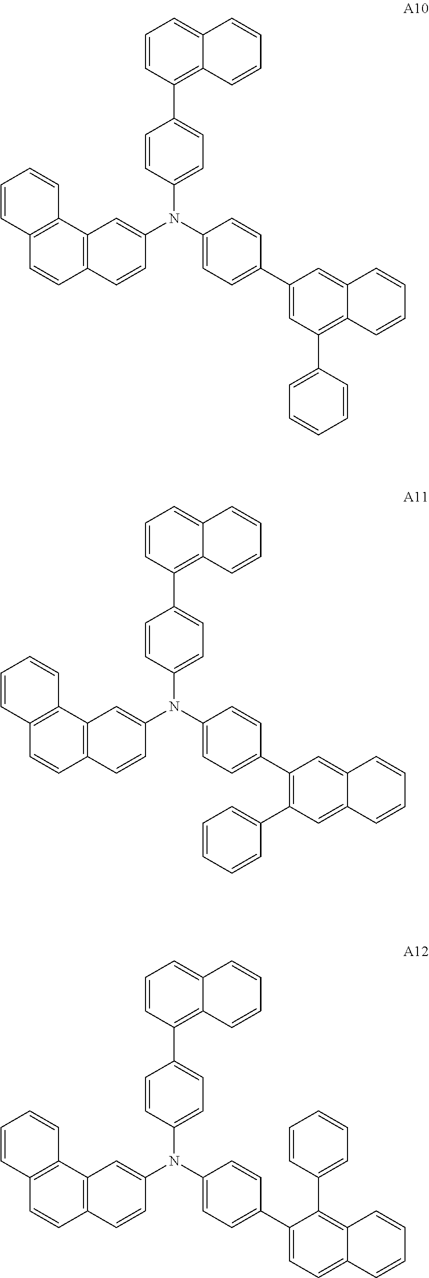 [SMILES: c1ccc(-c2cc(-c3ccc(N(c4ccc(-c5cccc6ccccc56)cc4)c4ccc5ccc6ccccc6c5c4)cc3)cc3ccccc23)cc1 ; c1ccc(-c2cc3ccccc3cc2-c2ccc(N(c3ccc(-c4cccc5ccccc45)cc3)c3ccc4ccc5ccccc5c4c3)cc2)cc1 ; c1ccc(-c2c(-c3ccc(N(c4ccc(-c5cccc6ccccc56)cc4)c4ccc5ccc6ccccc6c5c4)cc3)ccc3ccccc23)cc1]

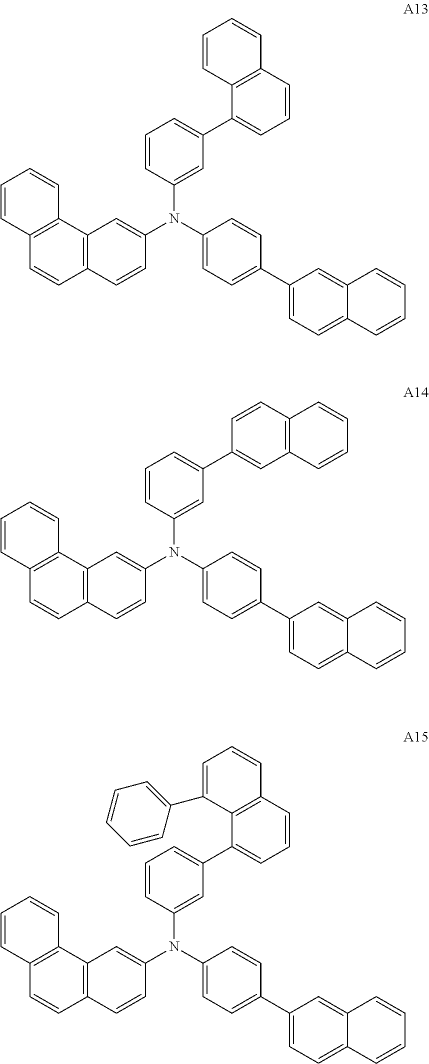 [SMILES: c1cc(-c2cccc3ccccc23)cc(N(c2ccc(-c3ccc4ccccc4c3)cc2)c2ccc3ccc4ccccc4c3c2)c1 ; c1cc(-c2ccc3ccccc3c2)cc(N(c2ccc(-c3ccc4ccccc4c3)cc2)c2ccc3ccc4ccccc4c3c2)c1 ; c1ccc(-c2cccc3cccc(-c4cccc(N(c5ccc(-c6ccc7ccccc7c6)cc5)c5ccc6ccc7ccccc7c6c5)c4)c23)cc1]

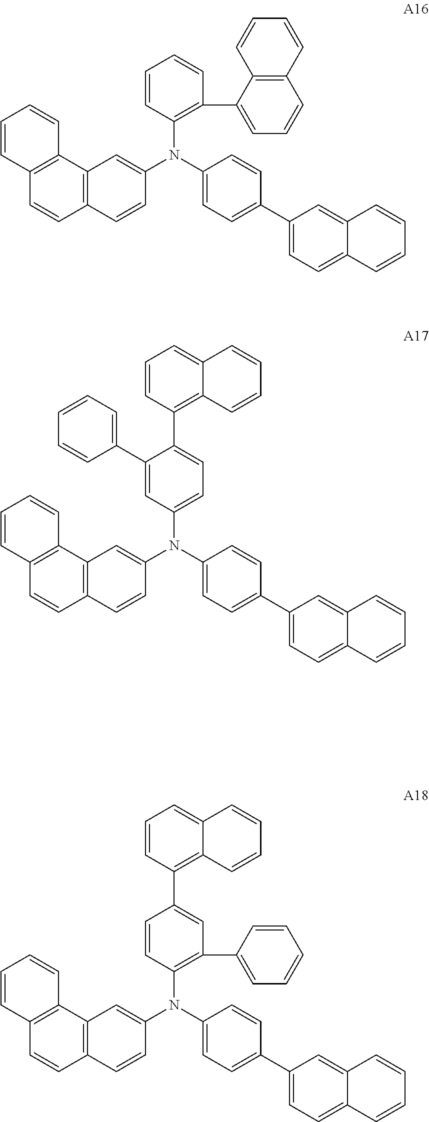 [SMILES: c1ccc(N(c2ccc(-c3ccc4ccccc4c3)cc2)c2ccc3ccc4ccccc4c3c2)c(-c2cccc3ccccc23)c1 ; c1ccc(-c2cc(N(c3ccc(-c4ccc5ccccc5c4)cc3)c3ccc4ccc5ccccc5c4c3)ccc2-c2cccc3ccccc23)cc1 ; c1ccc(-c2cc(-c3cccc4ccccc34)ccc2N(c2ccc(-c3ccc4ccccc4c3)cc2)c2ccc3ccc4ccccc4c3c2)cc1]

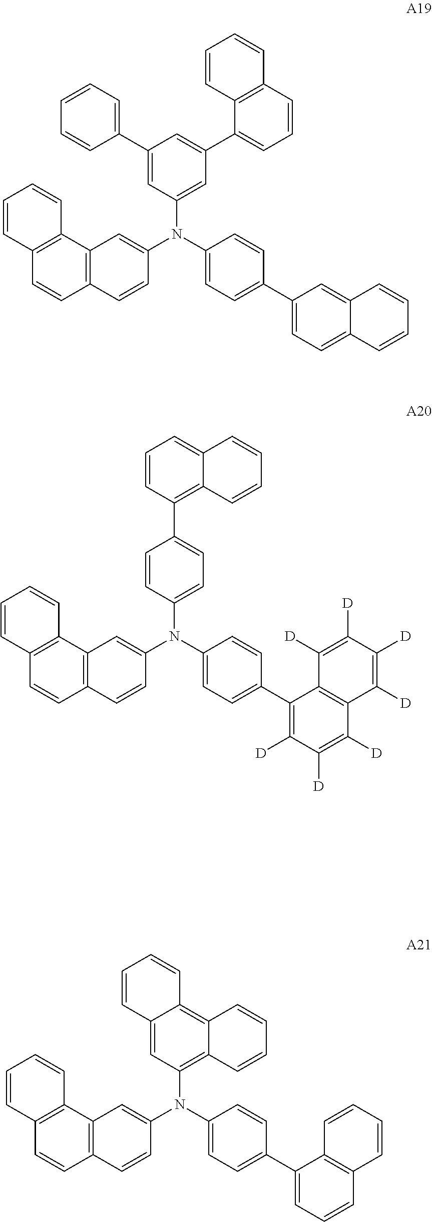 [SMILES: c1ccc(-c2cc(-c3cccc4ccccc34)cc(N(c3ccc(-c4ccc5ccccc5c4)cc3)c3ccc4ccc5ccccc5c4c3)c2)cc1 ; [2H]c1c([2H])c([2H])c2c(-c3ccc(N(c4ccc(-c5cccc6ccccc56)cc4)c4ccc5ccc6ccccc6c5c4)cc3)c([2H])c([2H])c([2H])c2c1[2H] ; c1ccc2c(-c3ccc(N(c4ccc5ccc6ccccc6c5c4)c4cc5ccccc5c5ccccc45)cc3)cccc2c1]

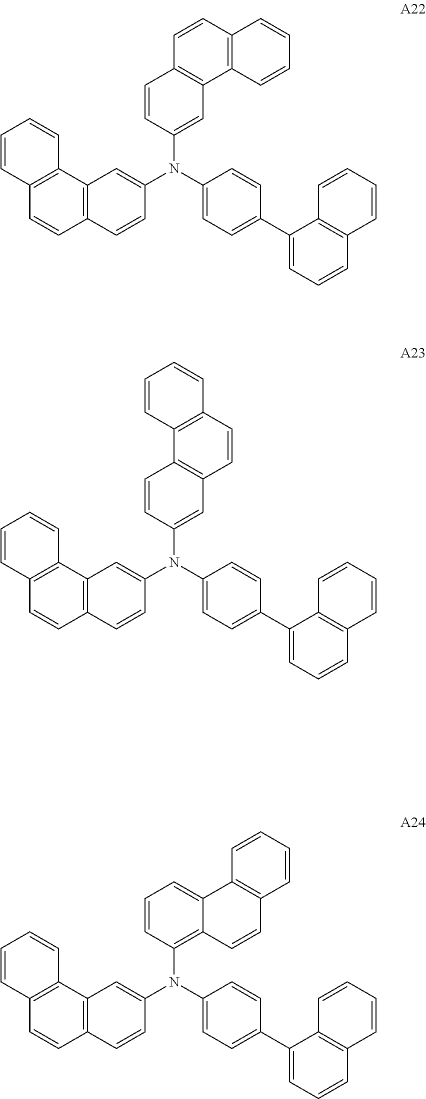 [SMILES: c1ccc2c(-c3ccc(N(c4ccc5ccc6ccccc6c5c4)c4ccc5ccc6ccccc6c5c4)cc3)cccc2c1 ; c1ccc2c(-c3ccc(N(c4ccc5c(ccc6ccccc65)c4)c4ccc5ccc6ccccc6c5c4)cc3)cccc2c1 ; c1ccc2c(-c3ccc(N(c4ccc5ccc6ccccc6c5c4)c4cccc5c4ccc4ccccc45)cc3)cccc2c1]

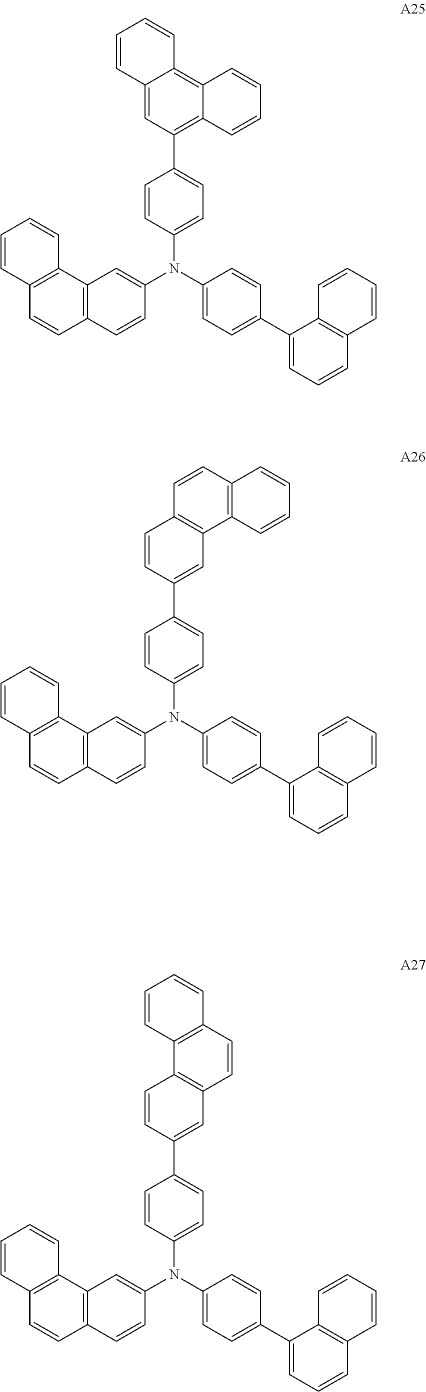 [SMILES: c1ccc2c(-c3ccc(N(c4ccc(-c5cc6ccccc6c6ccccc56)cc4)c4ccc5ccc6ccccc6c5c4)cc3)cccc2c1 ; c1ccc2c(-c3ccc(N(c4ccc(-c5ccc6ccc7ccccc7c6c5)cc4)c4ccc5ccc6ccccc6c5c4)cc3)cccc2c1 ; c1ccc2c(-c3ccc(N(c4ccc(-c5ccc6c(ccc7ccccc76)c5)cc4)c4ccc5ccc6ccccc6c5c4)cc3)cccc2c1]

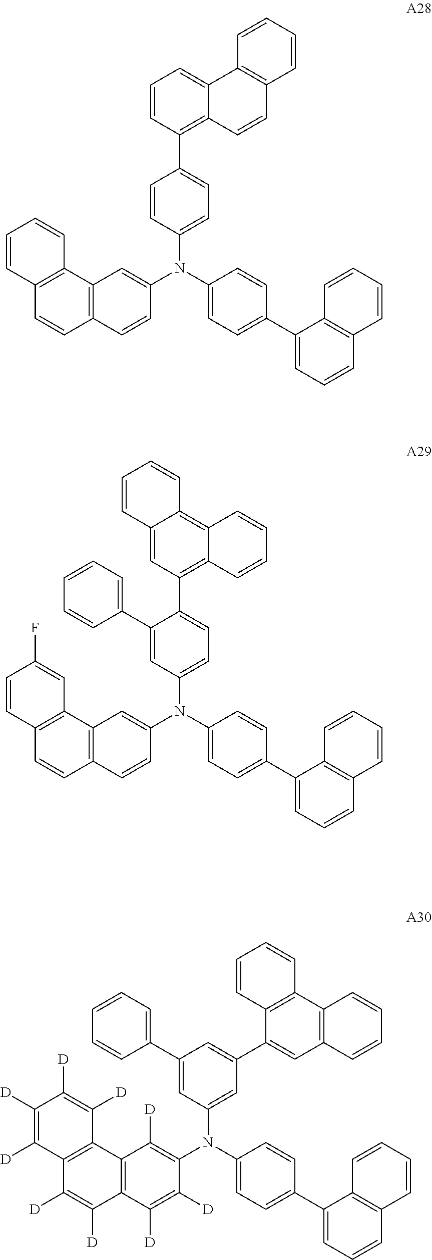 [SMILES: c1ccc2c(-c3ccc(N(c4ccc(-c5cccc6c5ccc5ccccc56)cc4)c4ccc5ccc6ccccc6c5c4)cc3)cccc2c1 ; Fc1ccc2ccc3ccc(N(c4ccc(-c5cccc6ccccc56)cc4)c4ccc(-c5cc6ccccc6c6ccccc56)c(-c5ccccc5)c4)cc3c2c1 ; [2H]c1c([2H])c([2H])c2c(c1[2H])c([2H])c([2H])c1c([2H])c([2H])c(N(c3ccc(-c4cccc5ccccc45)cc3)c3cc(-c4ccccc4)cc(-c4cc5ccccc5c5ccccc45)c3)c([2H])c12]

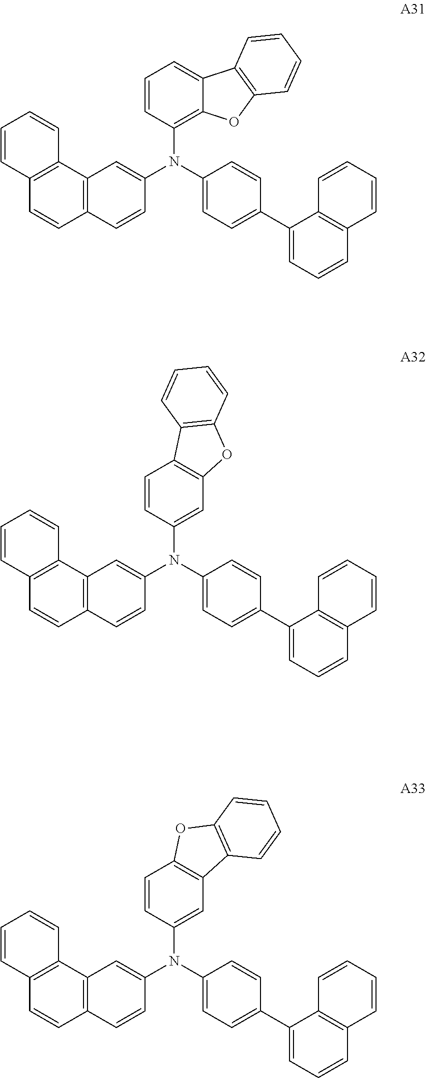 [SMILES: c1ccc2c(-c3ccc(N(c4ccc5ccc6ccccc6c5c4)c4cccc5c4oc4ccccc45)cc3)cccc2c1 ; c1ccc2c(-c3ccc(N(c4ccc5c(c4)oc4ccccc45)c4ccc5ccc6ccccc6c5c4)cc3)cccc2c1 ; c1ccc2c(-c3ccc(N(c4ccc5ccc6ccccc6c5c4)c4ccc5oc6ccccc6c5c4)cc3)cccc2c1]

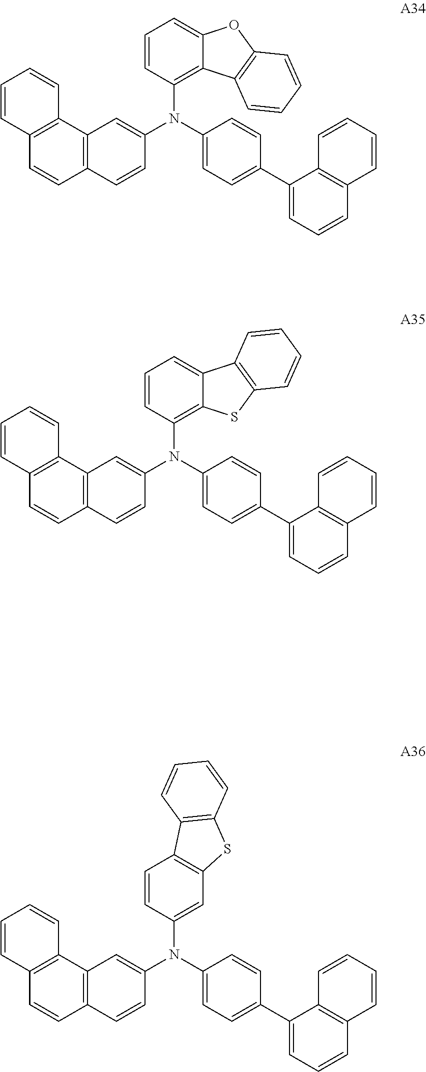 [SMILES: c1ccc2c(-c3ccc(N(c4ccc5ccc6ccccc6c5c4)c4cccc5oc6ccccc6c45)cc3)cccc2c1 ; c1ccc2c(-c3ccc(N(c4ccc5ccc6ccccc6c5c4)c4cccc5c4sc4ccccc45)cc3)cccc2c1 ; c1ccc2c(-c3ccc(N(c4ccc5c(c4)sc4ccccc45)c4ccc5ccc6ccccc6c5c4)cc3)cccc2c1]

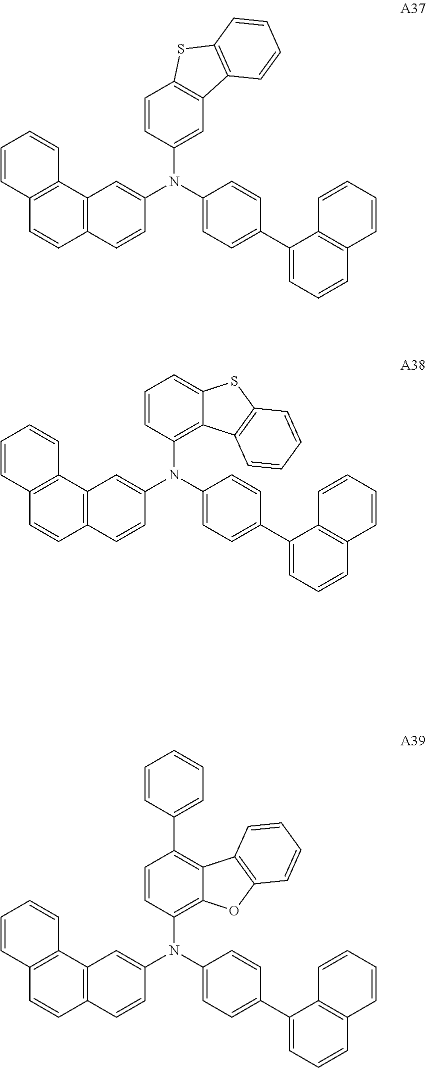 [SMILES: c1ccc2c(-c3ccc(N(c4ccc5ccc6ccccc6c5c4)c4ccc5sc6ccccc6c5c4)cc3)cccc2c1 ; c1ccc2c(-c3ccc(N(c4ccc5ccc6ccccc6c5c4)c4cccc5sc6ccccc6c45)cc3)cccc2c1 ; c1ccc(-c2ccc(N(c3ccc(-c4cccc5ccccc45)cc3)c3ccc4ccc5ccccc5c4c3)c3oc4ccccc4c23)cc1]

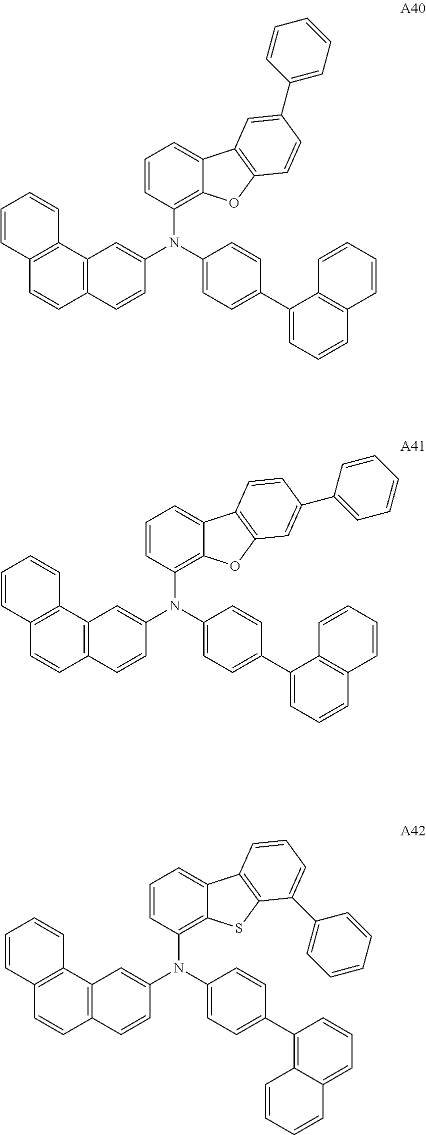 [SMILES: c1ccc(-c2ccc3oc4c(N(c5ccc(-c6cccc7ccccc67)cc5)c5ccc6ccc7ccccc7c6c5)cccc4c3c2)cc1 ; c1ccc(-c2ccc3c(c2)oc2c(N(c4ccc(-c5cccc6ccccc56)cc4)c4ccc5ccc6ccccc6c5c4)cccc23)cc1 ; c1ccc(-c2cccc3c2sc2c(N(c4ccc(-c5cccc6ccccc56)cc4)c4ccc5ccc6ccccc6c5c4)cccc23)cc1]

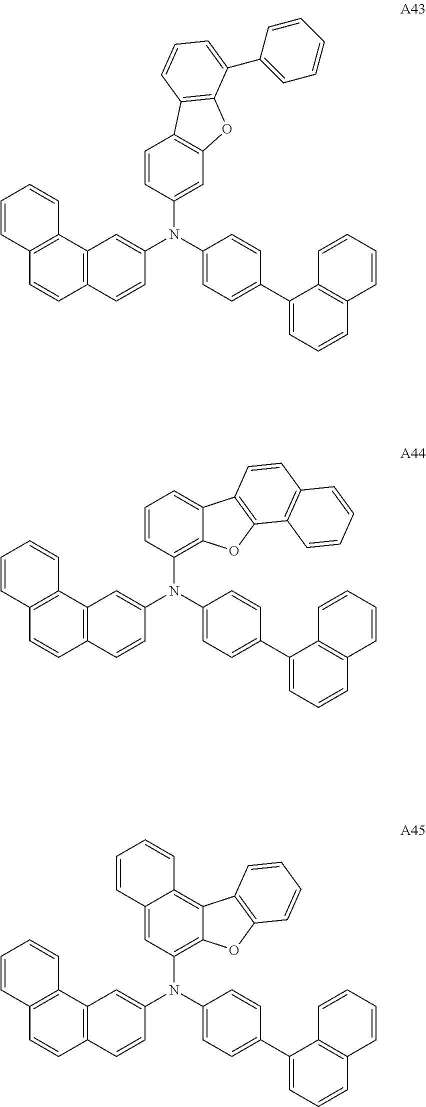 [SMILES: c1ccc(-c2cccc3c2oc2cc(N(c4ccc(-c5cccc6ccccc56)cc4)c4ccc5ccc6ccccc6c5c4)ccc23)cc1 ; c1ccc2c(-c3ccc(N(c4ccc5ccc6ccccc6c5c4)c4cccc5c4oc4c6ccccc6ccc54)cc3)cccc2c1 ; c1ccc2c(-c3ccc(N(c4ccc5ccc6ccccc6c5c4)c4cc5ccccc5c5c4oc4ccccc45)cc3)cccc2c1]

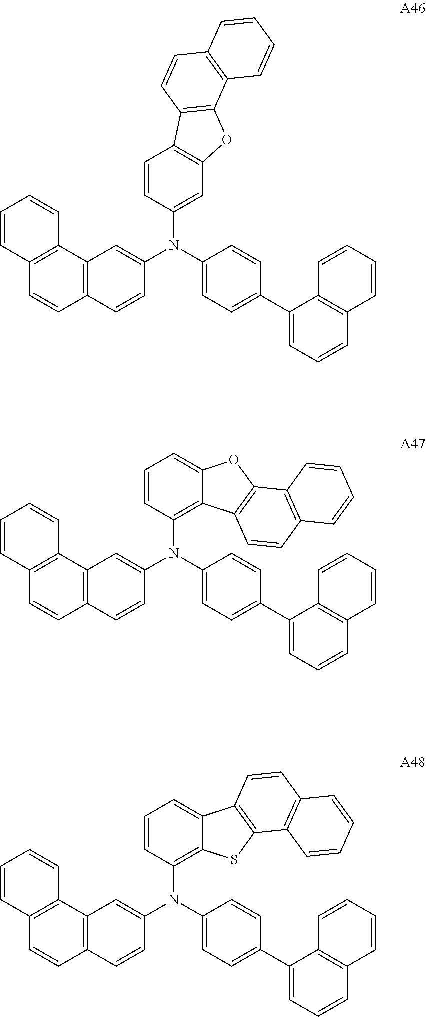 [SMILES: c1ccc2c(-c3ccc(N(c4ccc5c(c4)oc4c6ccccc6ccc54)c4ccc5ccc6ccccc6c5c4)cc3)cccc2c1 ; c1ccc2c(-c3ccc(N(c4ccc5ccc6ccccc6c5c4)c4cccc5oc6c7ccccc7ccc6c45)cc3)cccc2c1 ; c1ccc2c(-c3ccc(N(c4ccc5ccc6ccccc6c5c4)c4cccc5c4sc4c6ccccc6ccc54)cc3)cccc2c1]

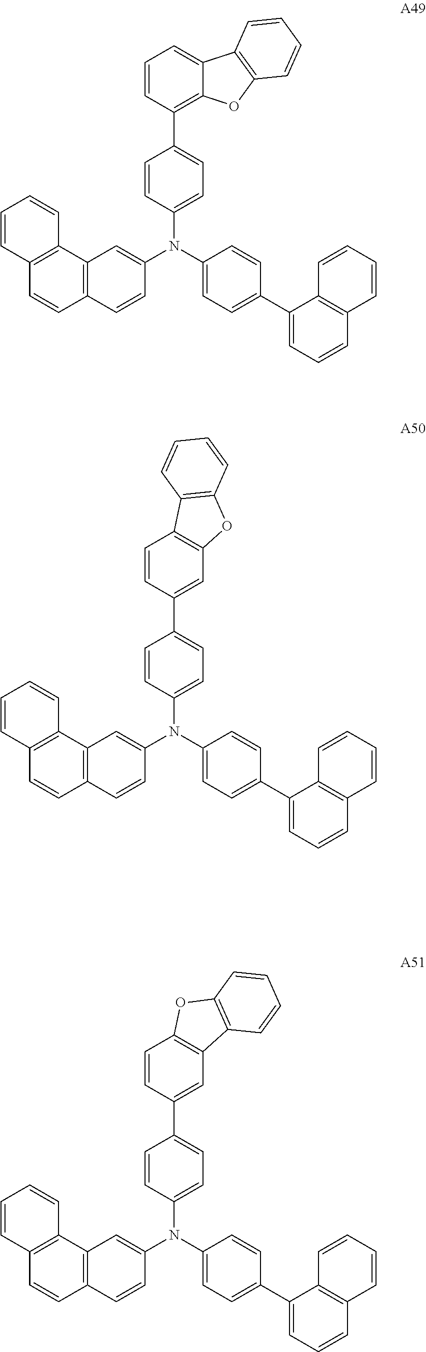 [SMILES: c1ccc2c(-c3ccc(N(c4ccc(-c5cccc6c5oc5ccccc56)cc4)c4ccc5ccc6ccccc6c5c4)cc3)cccc2c1 ; c1ccc2c(-c3ccc(N(c4ccc(-c5ccc6c(c5)oc5ccccc56)cc4)c4ccc5ccc6ccccc6c5c4)cc3)cccc2c1 ; c1ccc2c(-c3ccc(N(c4ccc(-c5ccc6oc7ccccc7c6c5)cc4)c4ccc5ccc6ccccc6c5c4)cc3)cccc2c1]

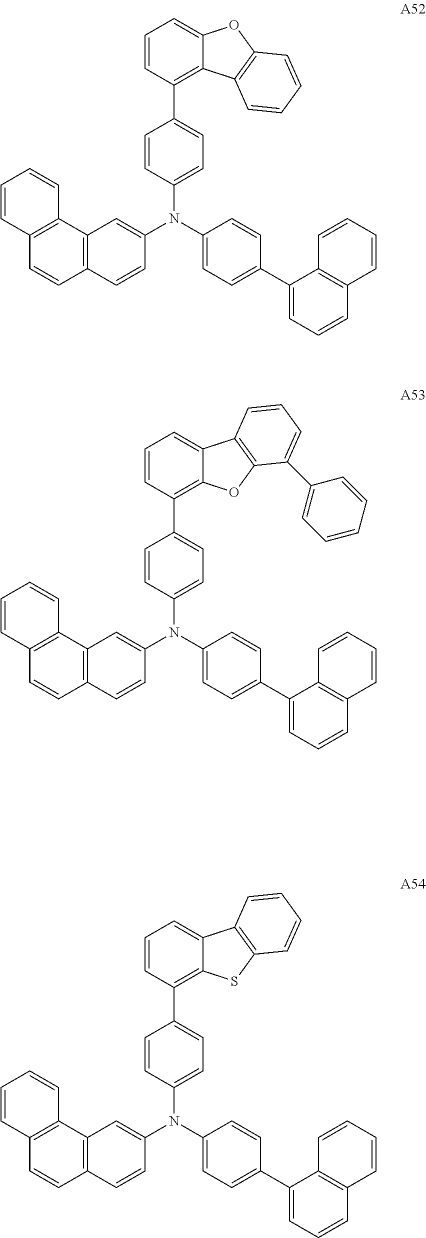 [SMILES: c1ccc2c(-c3ccc(N(c4ccc(-c5cccc6oc7ccccc7c56)cc4)c4ccc5ccc6ccccc6c5c4)cc3)cccc2c1 ; c1ccc(-c2cccc3c2oc2c(-c4ccc(N(c5ccc(-c6cccc7ccccc67)cc5)c5ccc6ccc7ccccc7c6c5)cc4)cccc23)cc1 ; c1ccc2c(-c3ccc(N(c4ccc(-c5cccc6c5sc5ccccc56)cc4)c4ccc5ccc6ccccc6c5c4)cc3)cccc2c1]

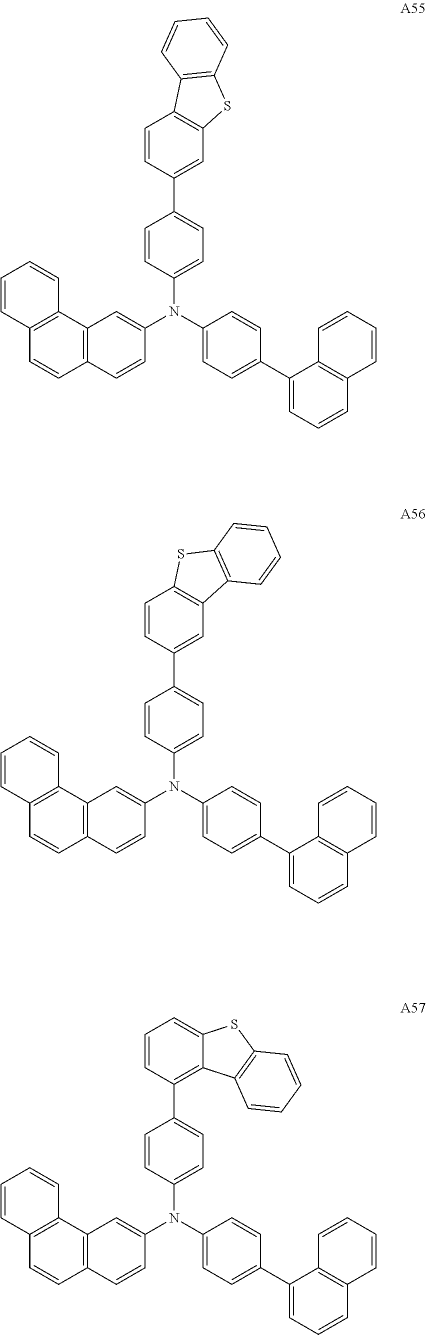 [SMILES: c1ccc2c(-c3ccc(N(c4ccc(-c5ccc6c(c5)sc5ccccc56)cc4)c4ccc5ccc6ccccc6c5c4)cc3)cccc2c1 ; c1ccc2c(-c3ccc(N(c4ccc(-c5ccc6sc7ccccc7c6c5)cc4)c4ccc5ccc6ccccc6c5c4)cc3)cccc2c1 ; c1ccc2c(-c3ccc(N(c4ccc(-c5cccc6sc7ccccc7c56)cc4)c4ccc5ccc6ccccc6c5c4)cc3)cccc2c1]

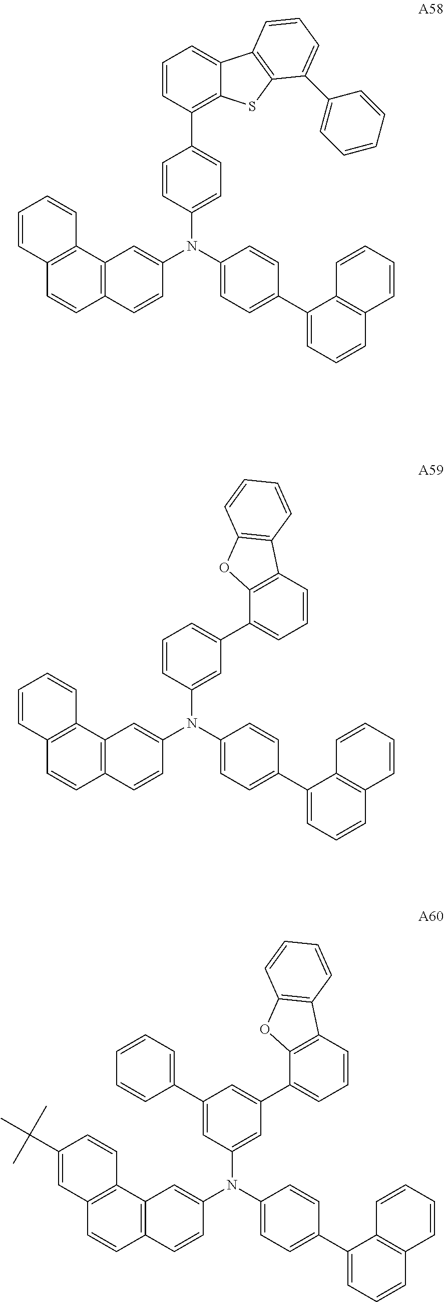 [SMILES: c1ccc(-c2cccc3c2sc2c(-c4ccc(N(c5ccc(-c6cccc7ccccc67)cc5)c5ccc6ccc7ccccc7c6c5)cc4)cccc23)cc1 ; c1cc(-c2cccc3c2oc2ccccc23)cc(N(c2ccc(-c3cccc4ccccc34)cc2)c2ccc3ccc4ccccc4c3c2)c1 ; CC(C)(C)c1ccc2c(ccc3ccc(N(c4ccc(-c5cccc6ccccc56)cc4)c4cc(-c5ccccc5)cc(-c5cccc6c5oc5ccccc56)c4)cc32)c1]

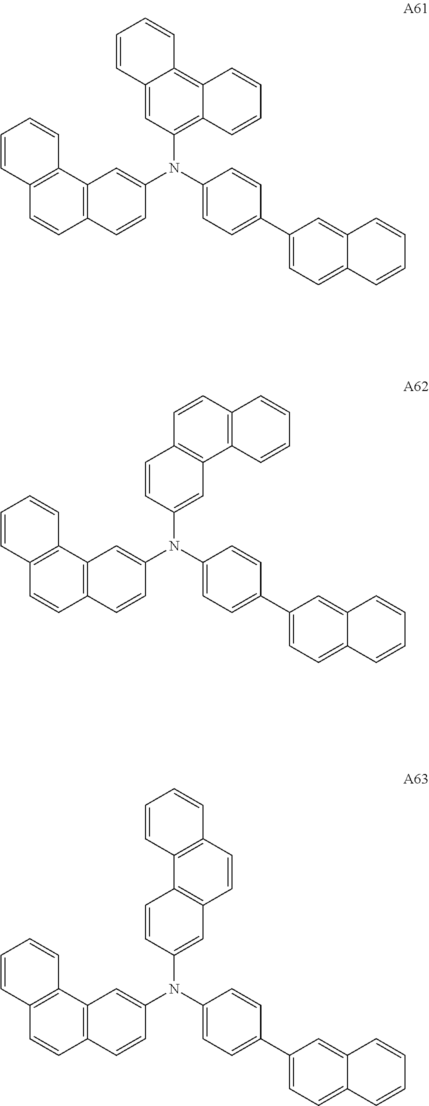 [SMILES: c1ccc2cc(-c3ccc(N(c4ccc5ccc6ccccc6c5c4)c4cc5ccccc5c5ccccc45)cc3)ccc2c1 ; c1ccc2cc(-c3ccc(N(c4ccc5ccc6ccccc6c5c4)c4ccc5ccc6ccccc6c5c4)cc3)ccc2c1 ; c1ccc2cc(-c3ccc(N(c4ccc5c(ccc6ccccc65)c4)c4ccc5ccc6ccccc6c5c4)cc3)ccc2c1]

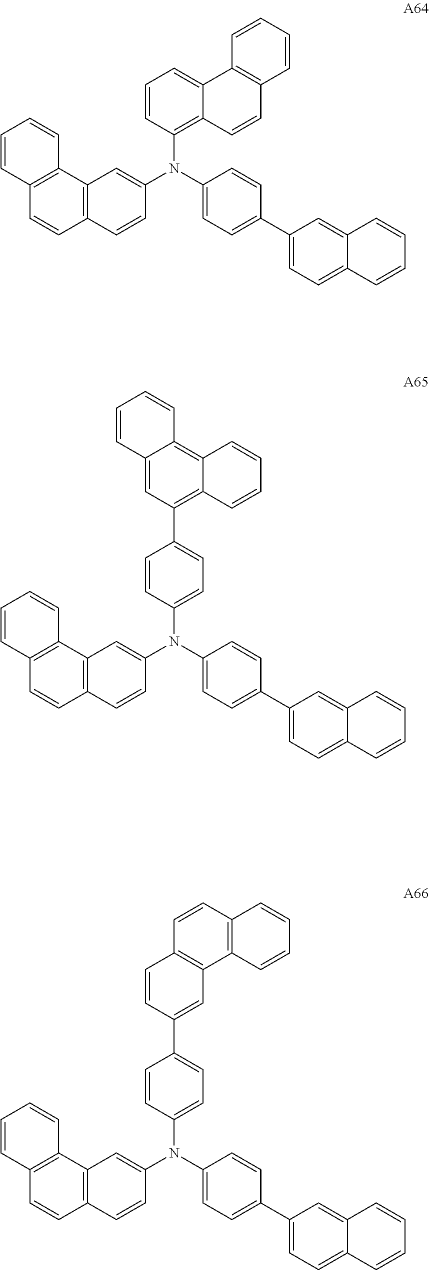 [SMILES: c1ccc2cc(-c3ccc(N(c4ccc5ccc6ccccc6c5c4)c4cccc5c4ccc4ccccc45)cc3)ccc2c1 ; c1ccc2cc(-c3ccc(N(c4ccc(-c5cc6ccccc6c6ccccc56)cc4)c4ccc5ccc6ccccc6c5c4)cc3)ccc2c1 ; c1ccc2cc(-c3ccc(N(c4ccc(-c5ccc6ccc7ccccc7c6c5)cc4)c4ccc5ccc6ccccc6c5c4)cc3)ccc2c1]

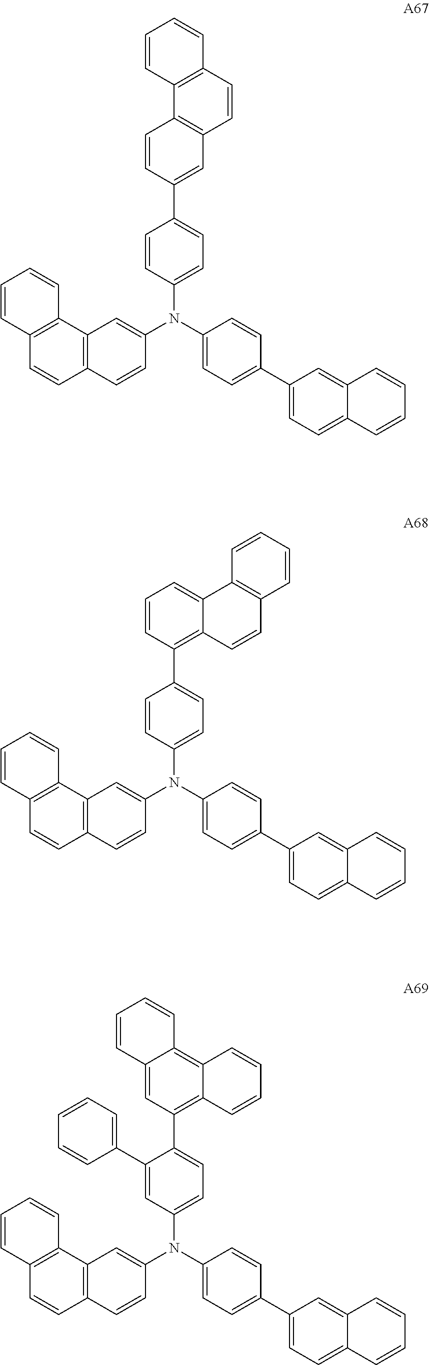 [SMILES: c1ccc2cc(-c3ccc(N(c4ccc(-c5ccc6c(ccc7ccccc76)c5)cc4)c4ccc5ccc6ccccc6c5c4)cc3)ccc2c1 ; c1ccc2cc(-c3ccc(N(c4ccc(-c5cccc6c5ccc5ccccc56)cc4)c4ccc5ccc6ccccc6c5c4)cc3)ccc2c1 ; c1ccc(-c2cc(N(c3ccc(-c4ccc5ccccc5c4)cc3)c3ccc4ccc5ccccc5c4c3)ccc2-c2cc3ccccc3c3ccccc23)cc1]

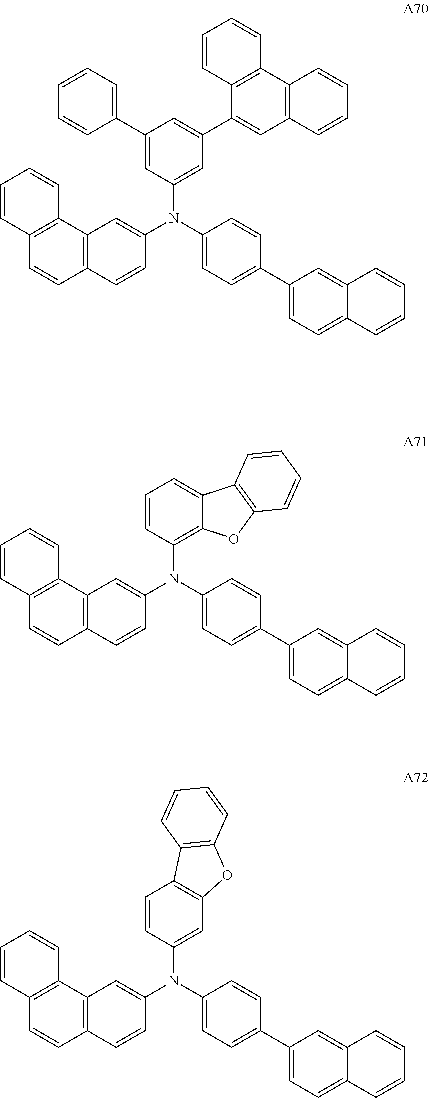 [SMILES: c1ccc(-c2cc(-c3cc4ccccc4c4ccccc34)cc(N(c3ccc(-c4ccc5ccccc5c4)cc3)c3ccc4ccc5ccccc5c4c3)c2)cc1 ; c1ccc2cc(-c3ccc(N(c4ccc5ccc6ccccc6c5c4)c4cccc5c4oc4ccccc45)cc3)ccc2c1 ; c1ccc2cc(-c3ccc(N(c4ccc5c(c4)oc4ccccc45)c4ccc5ccc6ccccc6c5c4)cc3)ccc2c1]

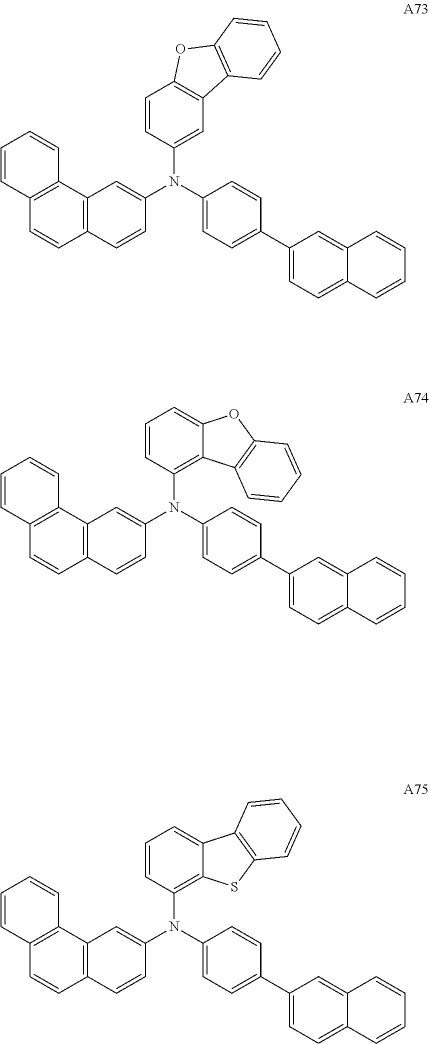 [SMILES: c1ccc2cc(-c3ccc(N(c4ccc5ccc6ccccc6c5c4)c4ccc5oc6ccccc6c5c4)cc3)ccc2c1 ; c1ccc2cc(-c3ccc(N(c4ccc5ccc6ccccc6c5c4)c4cccc5oc6ccccc6c45)cc3)ccc2c1 ; c1ccc2cc(-c3ccc(N(c4ccc5ccc6ccccc6c5c4)c4cccc5c4sc4ccccc45)cc3)ccc2c1]

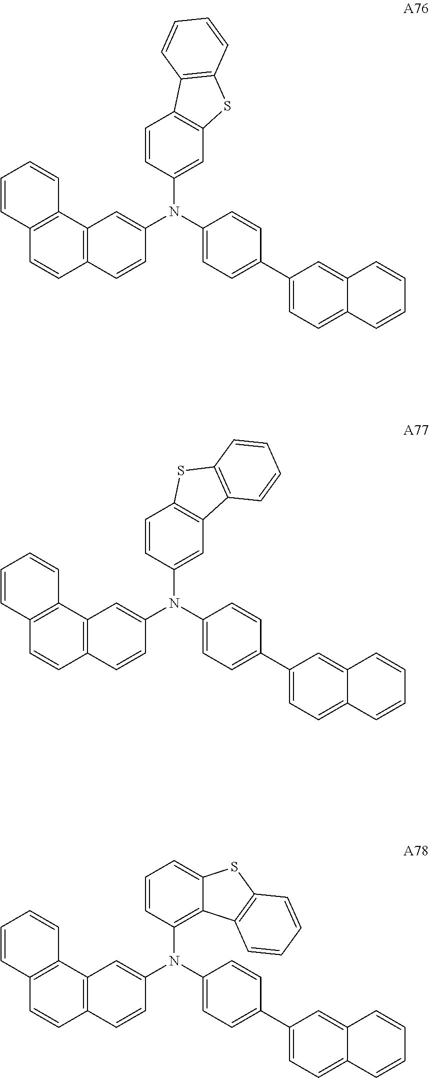 [SMILES: c1ccc2cc(-c3ccc(N(c4ccc5c(c4)sc4ccccc45)c4ccc5ccc6ccccc6c5c4)cc3)ccc2c1 ; c1ccc2cc(-c3ccc(N(c4ccc5ccc6ccccc6c5c4)c4ccc5sc6ccccc6c5c4)cc3)ccc2c1 ; c1ccc2cc(-c3ccc(N(c4ccc5ccc6ccccc6c5c4)c4cccc5sc6ccccc6c45)cc3)ccc2c1]

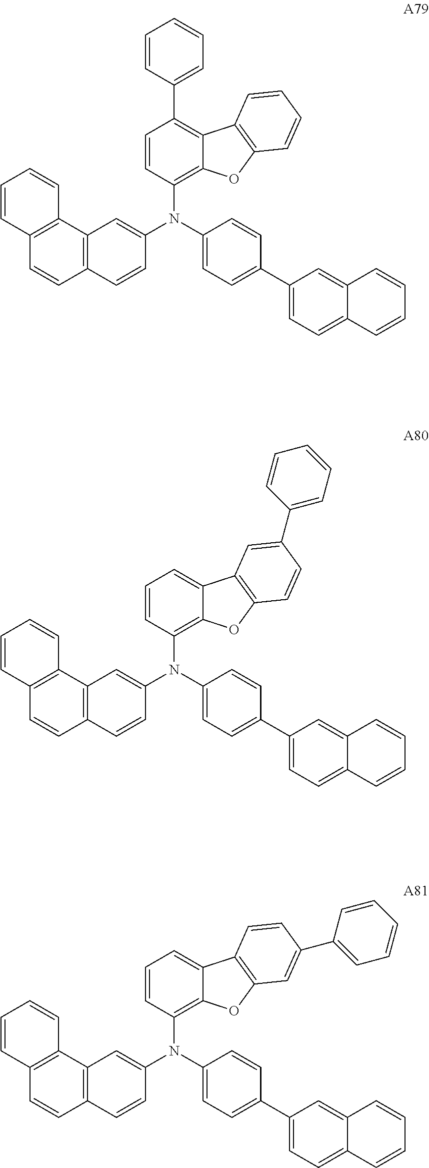 [SMILES: c1ccc(-c2ccc(N(c3ccc(-c4ccc5ccccc5c4)cc3)c3ccc4ccc5ccccc5c4c3)c3oc4ccccc4c23)cc1 ; c1ccc(-c2ccc3oc4c(N(c5ccc(-c6ccc7ccccc7c6)cc5)c5ccc6ccc7ccccc7c6c5)cccc4c3c2)cc1 ; c1ccc(-c2ccc3c(c2)oc2c(N(c4ccc(-c5ccc6ccccc6c5)cc4)c4ccc5ccc6ccccc6c5c4)cccc23)cc1]

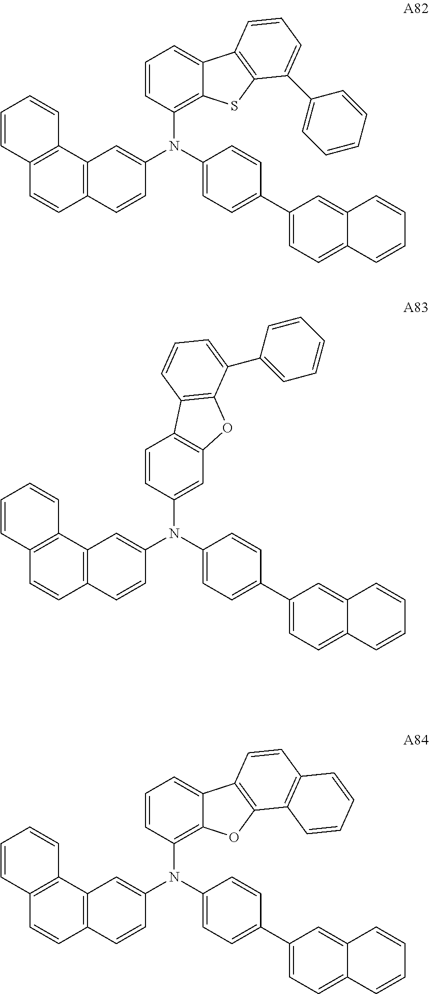 [SMILES: c1ccc(-c2cccc3c2sc2c(N(c4ccc(-c5ccc6ccccc6c5)cc4)c4ccc5ccc6ccccc6c5c4)cccc23)cc1 ; c1ccc(-c2cccc3c2oc2cc(N(c4ccc(-c5ccc6ccccc6c5)cc4)c4ccc5ccc6ccccc6c5c4)ccc23)cc1 ; c1ccc2cc(-c3ccc(N(c4ccc5ccc6ccccc6c5c4)c4cccc5c4oc4c6ccccc6ccc54)cc3)ccc2c1]

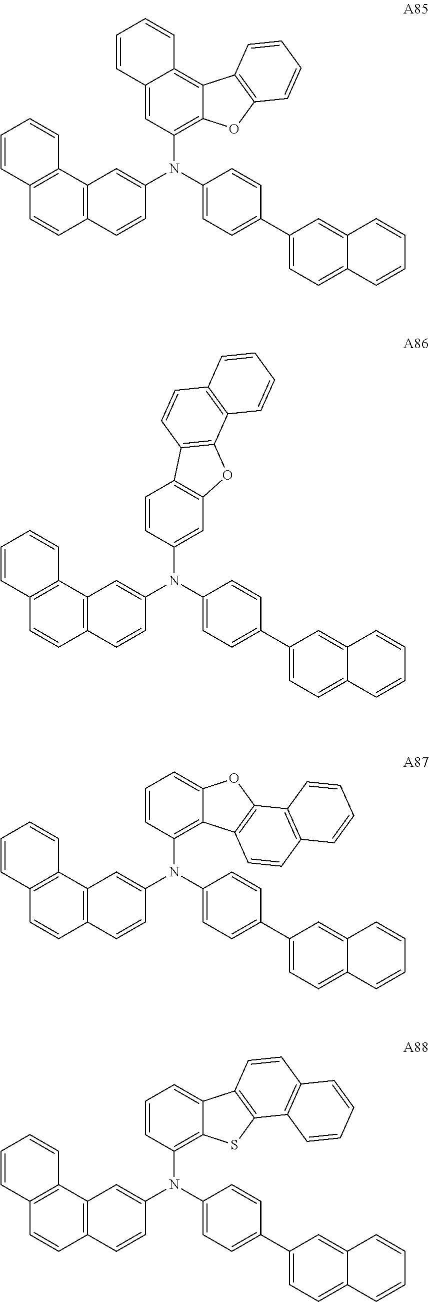 [SMILES: c1ccc2cc(-c3ccc(N(c4ccc5ccc6ccccc6c5c4)c4cc5ccccc5c5c4oc4ccccc45)cc3)ccc2c1 ; c1ccc2cc(-c3ccc(N(c4ccc5c(c4)oc4c6ccccc6ccc54)c4ccc5ccc6ccccc6c5c4)cc3)ccc2c1 ; c1ccc2cc(-c3ccc(N(c4ccc5ccc6ccccc6c5c4)c4cccc5oc6c7ccccc7ccc6c45)cc3)ccc2c1 ; c1ccc2cc(-c3ccc(N(c4ccc5ccc6ccccc6c5c4)c4cccc5c4sc4c6ccccc6ccc54)cc3)ccc2c1]

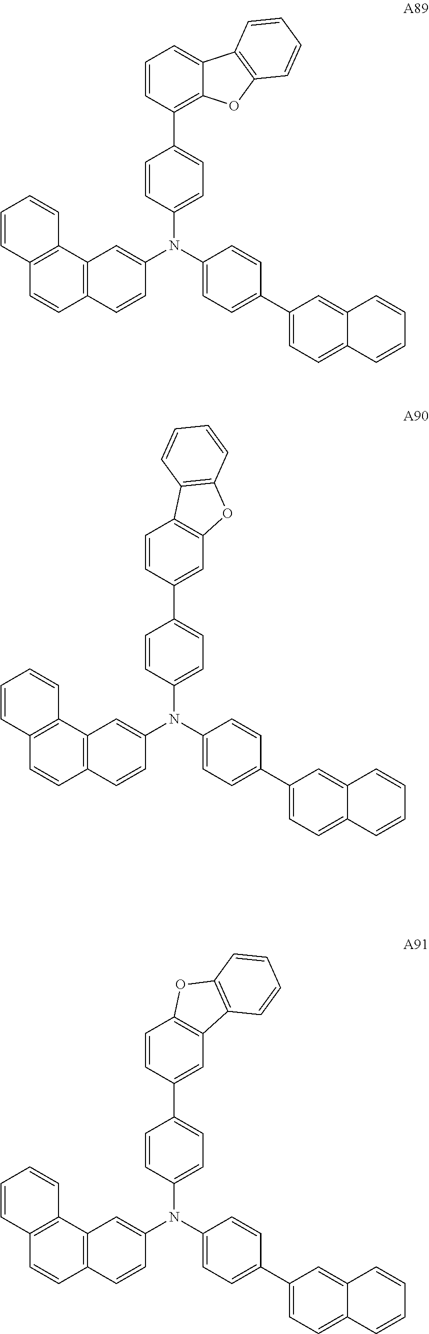 [SMILES: c1ccc2cc(-c3ccc(N(c4ccc(-c5cccc6c5oc5ccccc56)cc4)c4ccc5ccc6ccccc6c5c4)cc3)ccc2c1 ; c1ccc2cc(-c3ccc(N(c4ccc(-c5ccc6c(c5)oc5ccccc56)cc4)c4ccc5ccc6ccccc6c5c4)cc3)ccc2c1 ; c1ccc2cc(-c3ccc(N(c4ccc(-c5ccc6oc7ccccc7c6c5)cc4)c4ccc5ccc6ccccc6c5c4)cc3)ccc2c1]

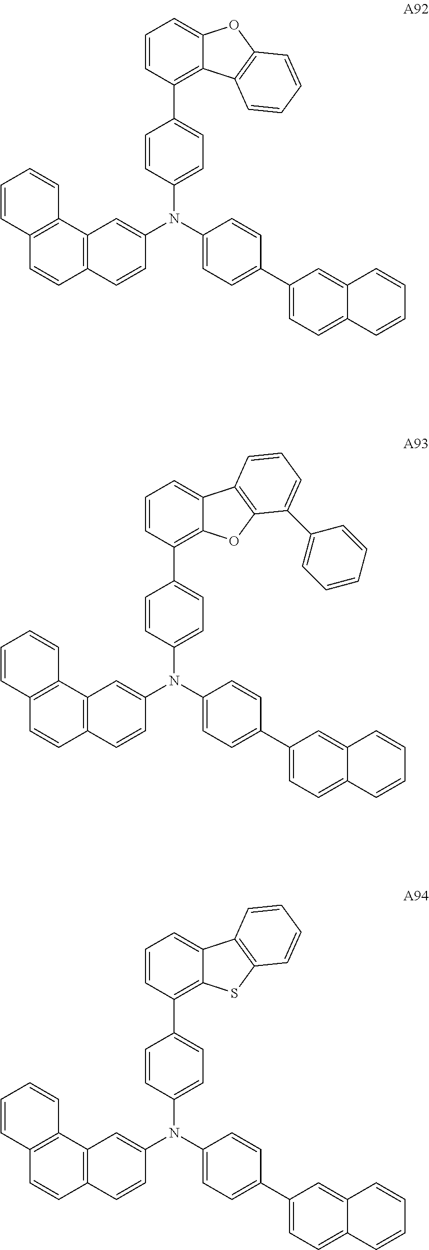 [SMILES: c1ccc2cc(-c3ccc(N(c4ccc(-c5cccc6oc7ccccc7c56)cc4)c4ccc5ccc6ccccc6c5c4)cc3)ccc2c1 ; c1ccc(-c2cccc3c2oc2c(-c4ccc(N(c5ccc(-c6ccc7ccccc7c6)cc5)c5ccc6ccc7ccccc7c6c5)cc4)cccc23)cc1 ; c1ccc2cc(-c3ccc(N(c4ccc(-c5cccc6c5sc5ccccc56)cc4)c4ccc5ccc6ccccc6c5c4)cc3)ccc2c1]

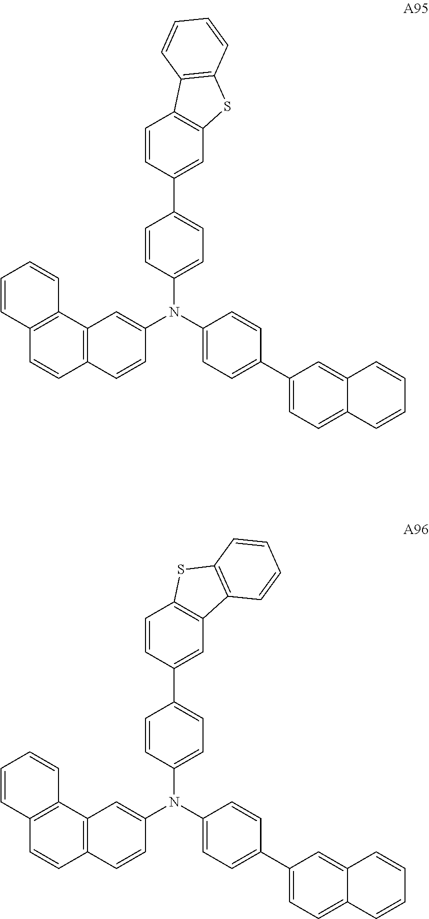 [SMILES: c1ccc2cc(-c3ccc(N(c4ccc(-c5ccc6c(c5)sc5ccccc56)cc4)c4ccc5ccc6ccccc6c5c4)cc3)ccc2c1 ; c1ccc2cc(-c3ccc(N(c4ccc(-c5ccc6sc7ccccc7c6c5)cc4)c4ccc5ccc6ccccc6c5c4)cc3)ccc2c1]

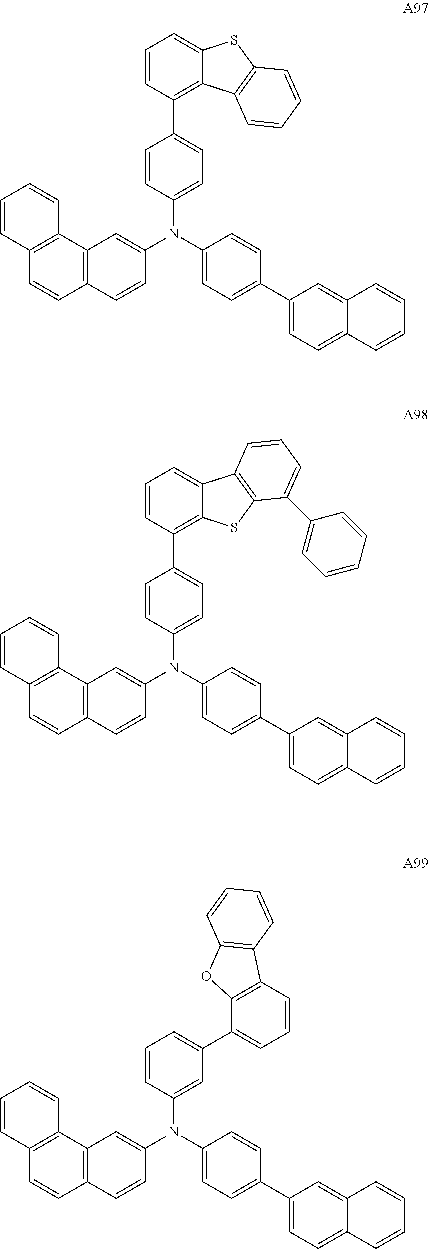 [SMILES: c1ccc2cc(-c3ccc(N(c4ccc(-c5cccc6sc7ccccc7c56)cc4)c4ccc5ccc6ccccc6c5c4)cc3)ccc2c1 ; c1ccc(-c2cccc3c2sc2c(-c4ccc(N(c5ccc(-c6ccc7ccccc7c6)cc5)c5ccc6ccc7ccccc7c6c5)cc4)cccc23)cc1 ; c1cc(-c2cccc3c2oc2ccccc23)cc(N(c2ccc(-c3ccc4ccccc4c3)cc2)c2ccc3ccc4ccccc4c3c2)c1]

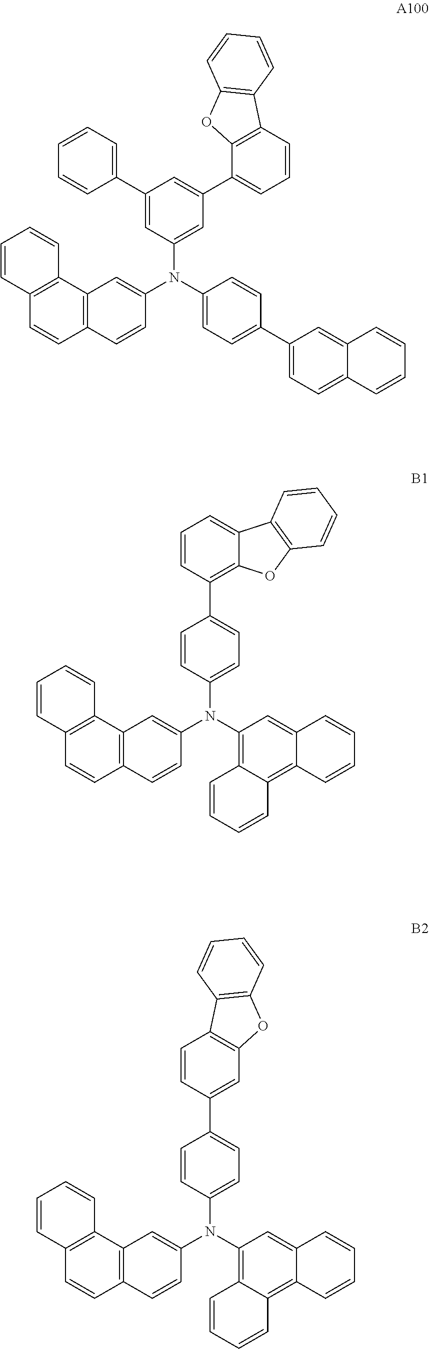 [SMILES: c1ccc(-c2cc(-c3cccc4c3oc3ccccc34)cc(N(c3ccc(-c4ccc5ccccc5c4)cc3)c3ccc4ccc5ccccc5c4c3)c2)cc1 ; c1ccc2c(c1)ccc1ccc(N(c3ccc(-c4cccc5c4oc4ccccc45)cc3)c3cc4ccccc4c4ccccc34)cc12 ; c1ccc2c(c1)ccc1ccc(N(c3ccc(-c4ccc5c(c4)oc4ccccc45)cc3)c3cc4ccccc4c4ccccc34)cc12]

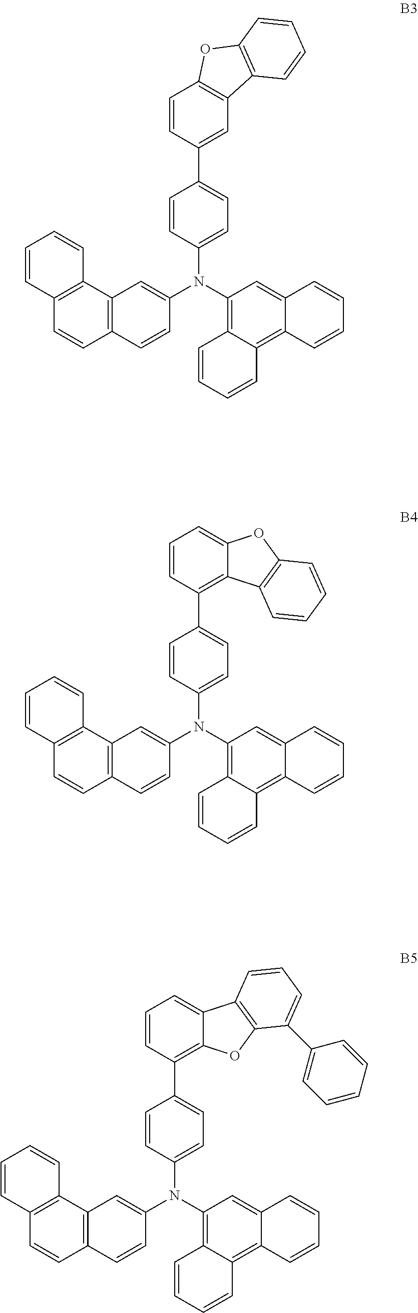 [SMILES: c1ccc2c(c1)ccc1ccc(N(c3ccc(-c4ccc5oc6ccccc6c5c4)cc3)c3cc4ccccc4c4ccccc34)cc12 ; c1ccc2c(c1)ccc1ccc(N(c3ccc(-c4cccc5oc6ccccc6c45)cc3)c3cc4ccccc4c4ccccc34)cc12 ; c1ccc(-c2cccc3c2oc2c(-c4ccc(N(c5ccc6ccc7ccccc7c6c5)c5cc6ccccc6c6ccccc56)cc4)cccc23)cc1]

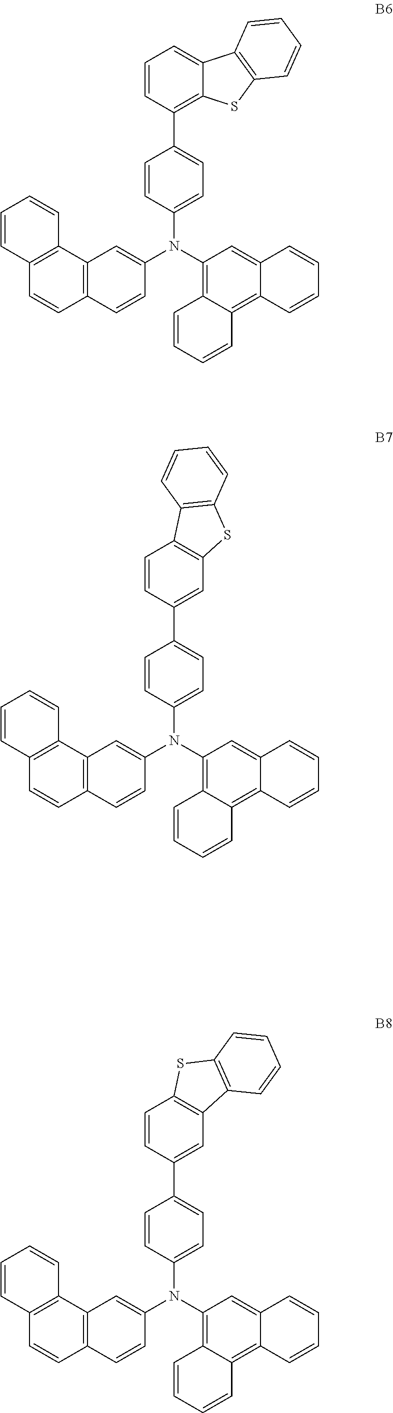 [SMILES: c1ccc2c(c1)ccc1ccc(N(c3ccc(-c4cccc5c4sc4ccccc45)cc3)c3cc4ccccc4c4ccccc34)cc12 ; c1ccc2c(c1)ccc1ccc(N(c3ccc(-c4ccc5c(c4)sc4ccccc45)cc3)c3cc4ccccc4c4ccccc34)cc12 ; c1ccc2c(c1)ccc1ccc(N(c3ccc(-c4ccc5sc6ccccc6c5c4)cc3)c3cc4ccccc4c4ccccc34)cc12]

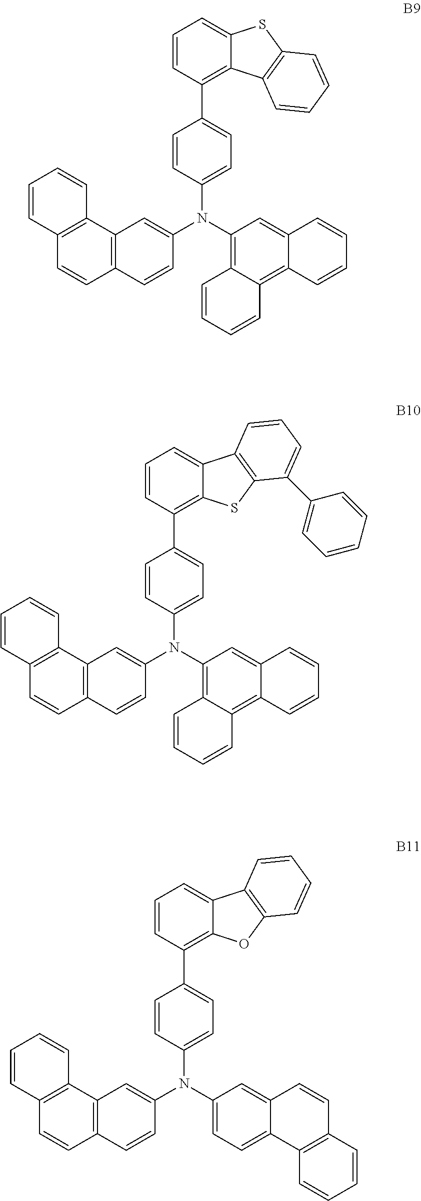 [SMILES: c1ccc2c(c1)ccc1ccc(N(c3ccc(-c4cccc5sc6ccccc6c45)cc3)c3cc4ccccc4c4ccccc34)cc12 ; c1ccc(-c2cccc3c2sc2c(-c4ccc(N(c5ccc6ccc7ccccc7c6c5)c5cc6ccccc6c6ccccc56)cc4)cccc23)cc1 ; c1ccc2c(c1)ccc1cc(N(c3ccc(-c4cccc5c4oc4ccccc45)cc3)c3ccc4ccc5ccccc5c4c3)ccc12]

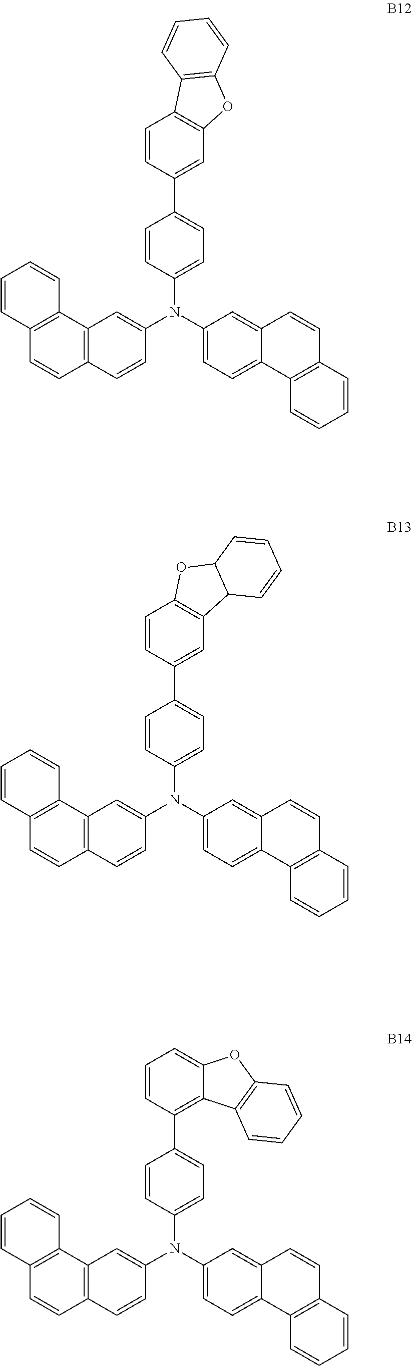 [SMILES: c1ccc2c(c1)ccc1cc(N(c3ccc(-c4ccc5c(c4)oc4ccccc45)cc3)c3ccc4ccc5ccccc5c4c3)ccc12 ; C1=CC2Oc3ccc(-c4ccc(N(c5ccc6c(ccc7ccccc76)c5)c5ccc6ccc7ccccc7c6c5)cc4)cc3C2C=C1 ; c1ccc2c(c1)ccc1cc(N(c3ccc(-c4cccc5oc6ccccc6c45)cc3)c3ccc4ccc5ccccc5c4c3)ccc12]

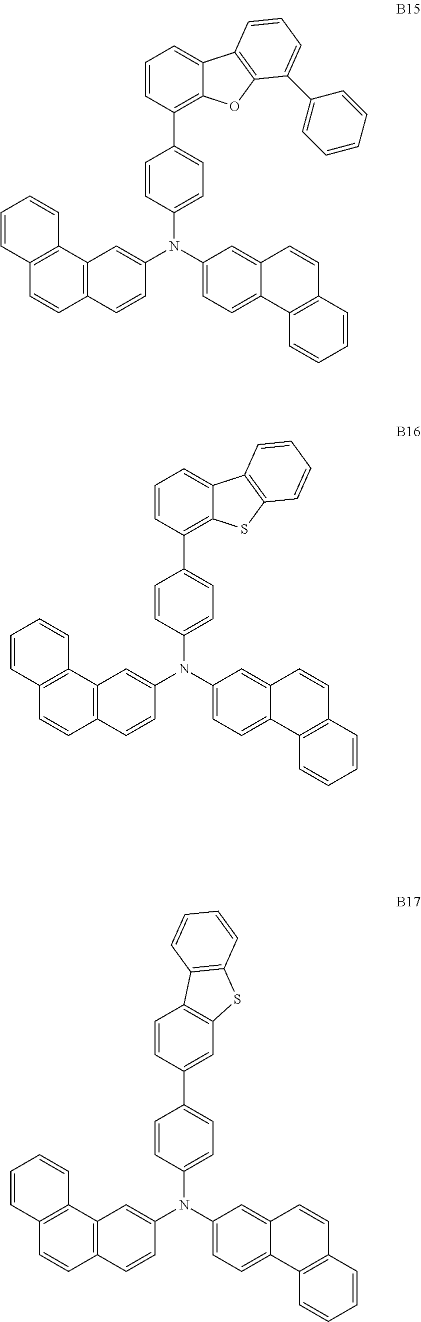 [SMILES: c1ccc(-c2cccc3c2oc2c(-c4ccc(N(c5ccc6c(ccc7ccccc76)c5)c5ccc6ccc7ccccc7c6c5)cc4)cccc23)cc1 ; c1ccc2c(c1)ccc1cc(N(c3ccc(-c4cccc5c4sc4ccccc45)cc3)c3ccc4ccc5ccccc5c4c3)ccc12 ; c1ccc2c(c1)ccc1cc(N(c3ccc(-c4ccc5c(c4)sc4ccccc45)cc3)c3ccc4ccc5ccccc5c4c3)ccc12]

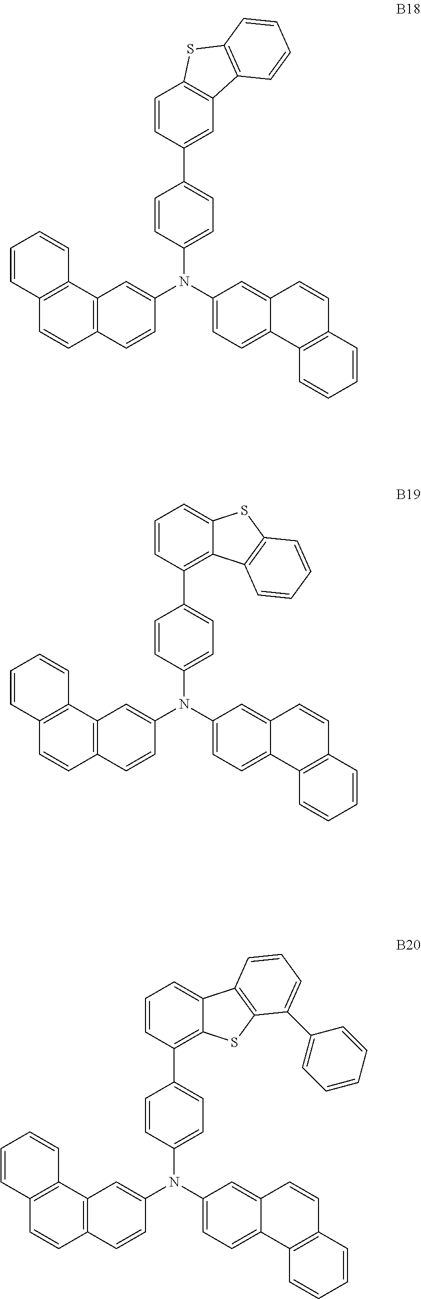 [SMILES: c1ccc2c(c1)ccc1cc(N(c3ccc(-c4ccc5sc6ccccc6c5c4)cc3)c3ccc4ccc5ccccc5c4c3)ccc12 ; c1ccc2c(c1)ccc1cc(N(c3ccc(-c4cccc5sc6ccccc6c45)cc3)c3ccc4ccc5ccccc5c4c3)ccc12 ; c1ccc(-c2cccc3c2sc2c(-c4ccc(N(c5ccc6c(ccc7ccccc76)c5)c5ccc6ccc7ccccc7c6c5)cc4)cccc23)cc1]

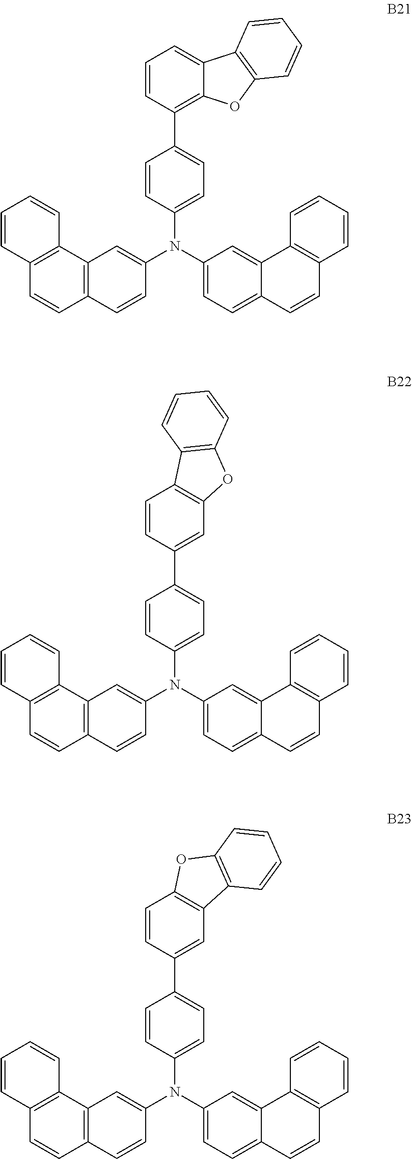 [SMILES: c1ccc2c(c1)ccc1ccc(N(c3ccc(-c4cccc5c4oc4ccccc45)cc3)c3ccc4ccc5ccccc5c4c3)cc12 ; c1ccc2c(c1)ccc1ccc(N(c3ccc(-c4ccc5c(c4)oc4ccccc45)cc3)c3ccc4ccc5ccccc5c4c3)cc12 ; c1ccc2c(c1)ccc1ccc(N(c3ccc(-c4ccc5oc6ccccc6c5c4)cc3)c3ccc4ccc5ccccc5c4c3)cc12]

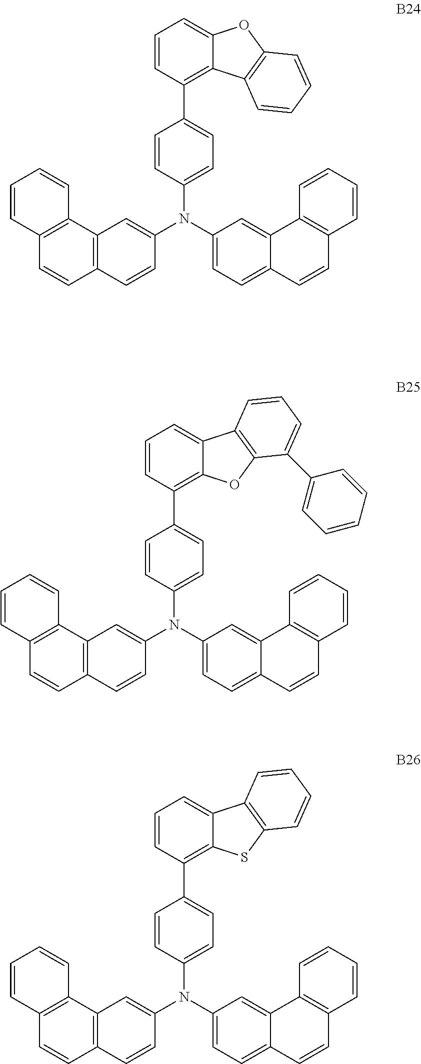 [SMILES: c1ccc2c(c1)ccc1ccc(N(c3ccc(-c4cccc5oc6ccccc6c45)cc3)c3ccc4ccc5ccccc5c4c3)cc12 ; c1ccc(-c2cccc3c2oc2c(-c4ccc(N(c5ccc6ccc7ccccc7c6c5)c5ccc6ccc7ccccc7c6c5)cc4)cccc23)cc1 ; c1ccc2c(c1)ccc1ccc(N(c3ccc(-c4cccc5c4sc4ccccc45)cc3)c3ccc4ccc5ccccc5c4c3)cc12]

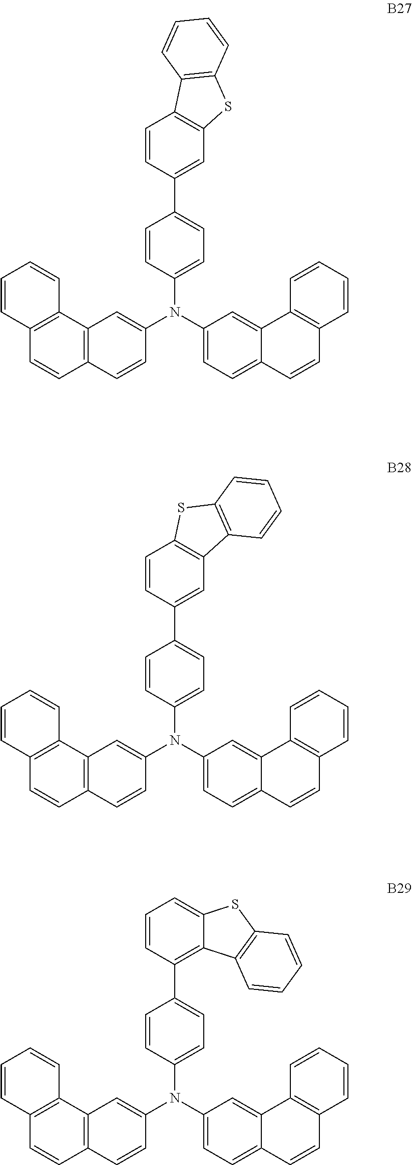 [SMILES: c1ccc2c(c1)ccc1ccc(N(c3ccc(-c4ccc5c(c4)sc4ccccc45)cc3)c3ccc4ccc5ccccc5c4c3)cc12 ; c1ccc2c(c1)ccc1ccc(N(c3ccc(-c4ccc5sc6ccccc6c5c4)cc3)c3ccc4ccc5ccccc5c4c3)cc12 ; c1ccc2c(c1)ccc1ccc(N(c3ccc(-c4cccc5sc6ccccc6c45)cc3)c3ccc4ccc5ccccc5c4c3)cc12]

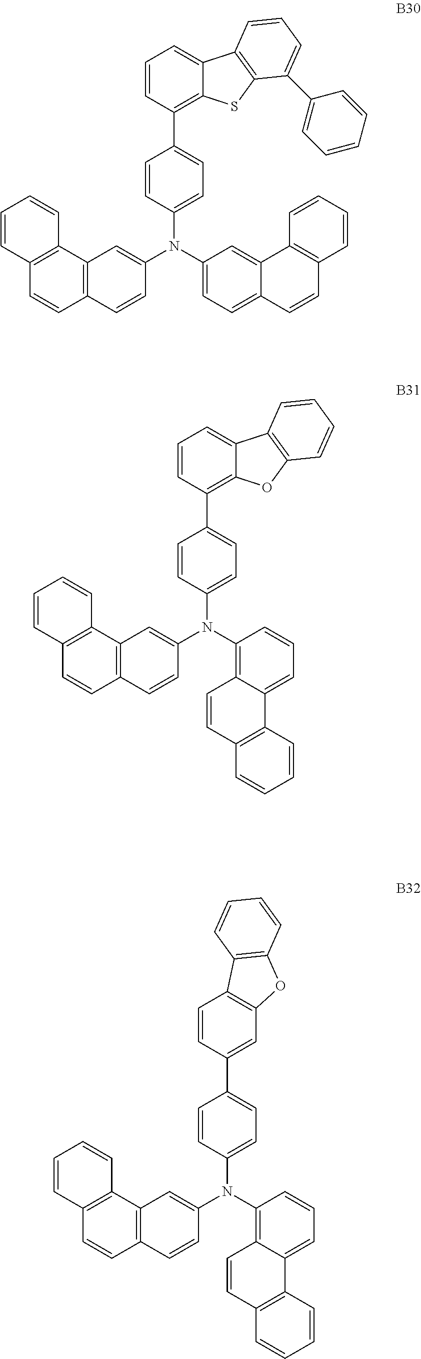 [SMILES: c1ccc(-c2cccc3c2sc2c(-c4ccc(N(c5ccc6ccc7ccccc7c6c5)c5ccc6ccc7ccccc7c6c5)cc4)cccc23)cc1 ; c1ccc2c(c1)ccc1ccc(N(c3ccc(-c4cccc5c4oc4ccccc45)cc3)c3cccc4c3ccc3ccccc34)cc12 ; c1ccc2c(c1)ccc1ccc(N(c3ccc(-c4ccc5c(c4)oc4ccccc45)cc3)c3cccc4c3ccc3ccccc34)cc12]

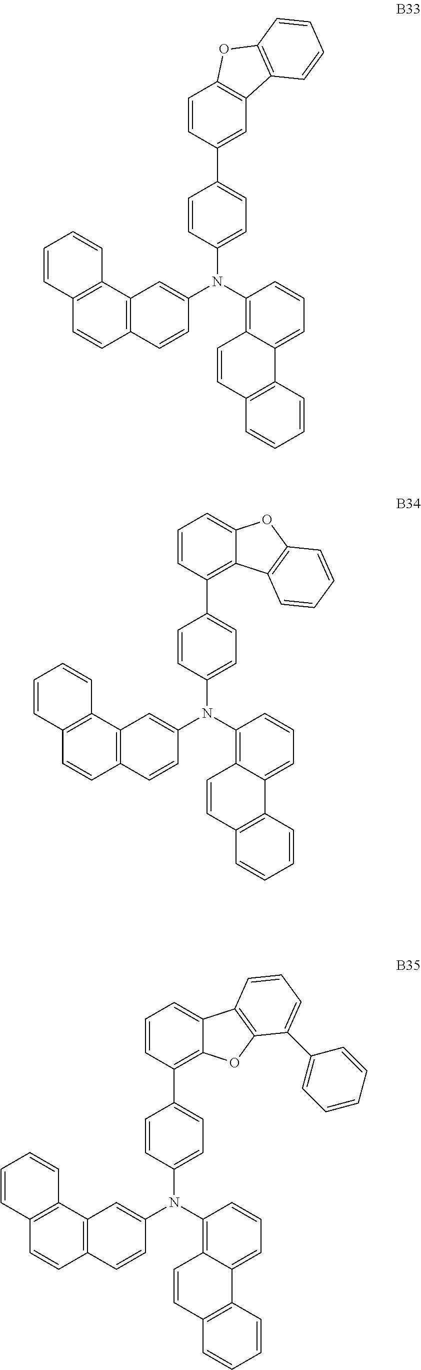 [SMILES: c1ccc2c(c1)ccc1ccc(N(c3ccc(-c4ccc5oc6ccccc6c5c4)cc3)c3cccc4c3ccc3ccccc34)cc12 ; c1ccc2c(c1)ccc1ccc(N(c3ccc(-c4cccc5oc6ccccc6c45)cc3)c3cccc4c3ccc3ccccc34)cc12 ; c1ccc(-c2cccc3c2oc2c(-c4ccc(N(c5ccc6ccc7ccccc7c6c5)c5cccc6c5ccc5ccccc56)cc4)cccc23)cc1]

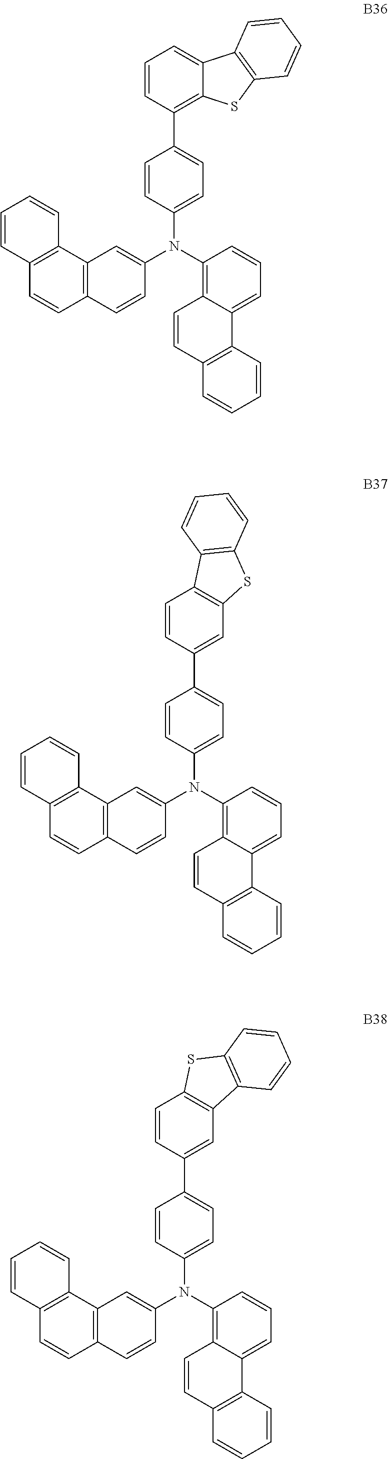 [SMILES: c1ccc2c(c1)ccc1ccc(N(c3ccc(-c4cccc5c4sc4ccccc45)cc3)c3cccc4c3ccc3ccccc34)cc12 ; c1ccc2c(c1)ccc1ccc(N(c3ccc(-c4ccc5c(c4)sc4ccccc45)cc3)c3cccc4c3ccc3ccccc34)cc12 ; c1ccc2c(c1)ccc1ccc(N(c3ccc(-c4ccc5sc6ccccc6c5c4)cc3)c3cccc4c3ccc3ccccc34)cc12]

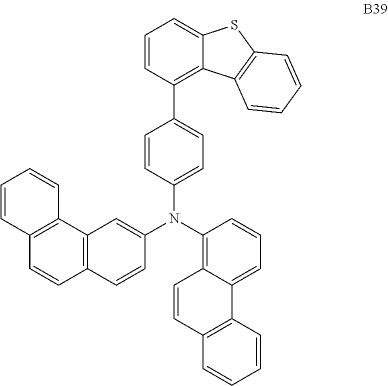 [SMILES: c1ccc2c(c1)ccc1ccc(N(c3ccc(-c4cccc5sc6ccccc6c45)cc3)c3cccc4c3ccc3ccccc34)cc12]

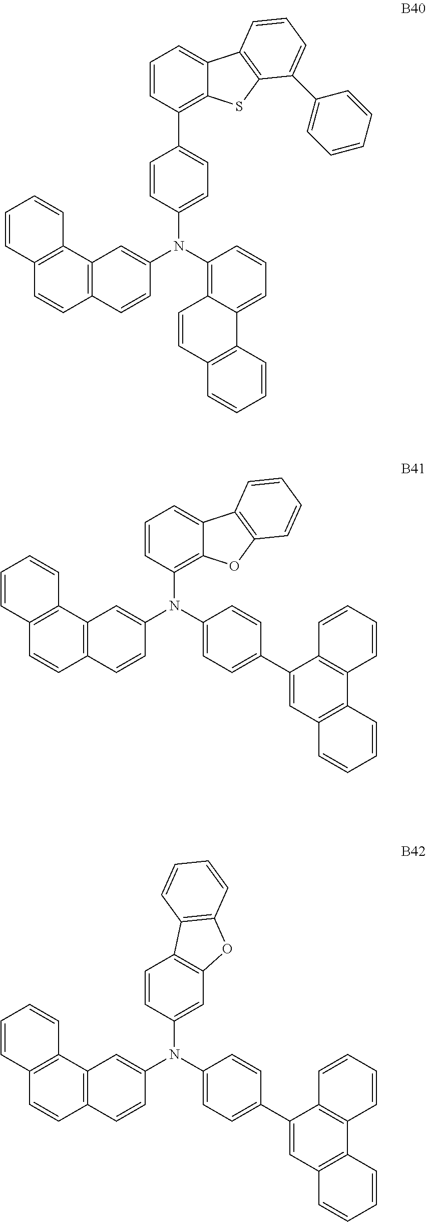 [SMILES: c1ccc(-c2cccc3c2sc2c(-c4ccc(N(c5ccc6ccc7ccccc7c6c5)c5cccc6c5ccc5ccccc56)cc4)cccc23)cc1 ; c1ccc2c(c1)ccc1ccc(N(c3ccc(-c4cc5ccccc5c5ccccc45)cc3)c3cccc4c3oc3ccccc34)cc12 ; c1ccc2c(c1)ccc1ccc(N(c3ccc(-c4cc5ccccc5c5ccccc45)cc3)c3ccc4c(c3)oc3ccccc34)cc12]

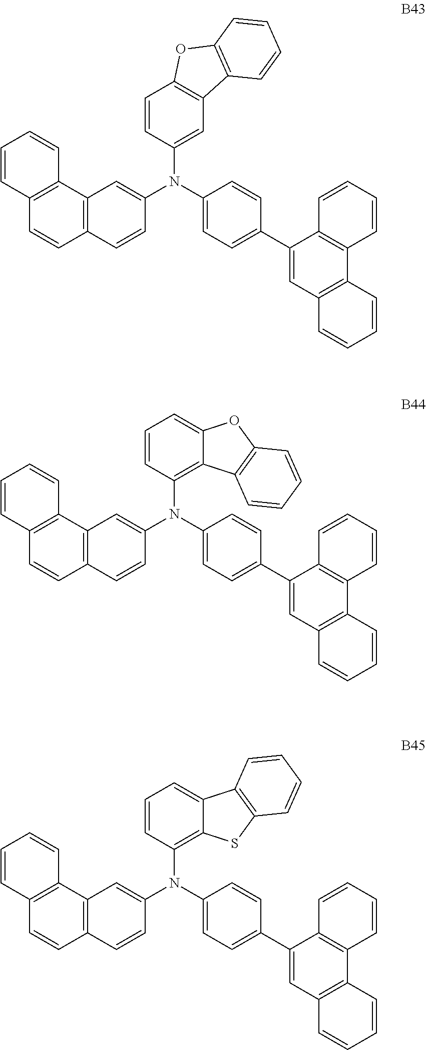 [SMILES: c1ccc2c(c1)ccc1ccc(N(c3ccc(-c4cc5ccccc5c5ccccc45)cc3)c3ccc4oc5ccccc5c4c3)cc12 ; c1ccc2c(c1)ccc1ccc(N(c3ccc(-c4cc5ccccc5c5ccccc45)cc3)c3cccc4oc5ccccc5c34)cc12 ; c1ccc2c(c1)ccc1ccc(N(c3ccc(-c4cc5ccccc5c5ccccc45)cc3)c3cccc4c3sc3ccccc34)cc12]

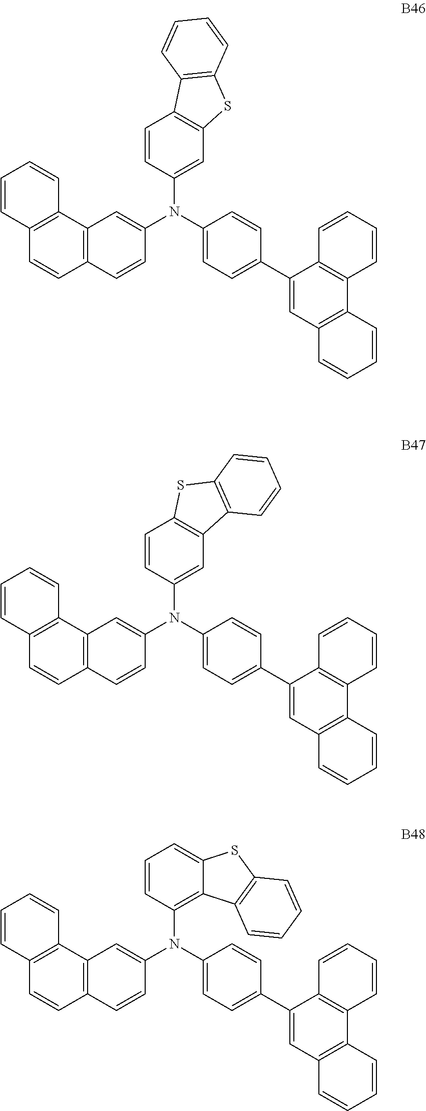 [SMILES: c1ccc2c(c1)ccc1ccc(N(c3ccc(-c4cc5ccccc5c5ccccc45)cc3)c3ccc4c(c3)sc3ccccc34)cc12 ; c1ccc2c(c1)ccc1ccc(N(c3ccc(-c4cc5ccccc5c5ccccc45)cc3)c3ccc4sc5ccccc5c4c3)cc12 ; c1ccc2c(c1)ccc1ccc(N(c3ccc(-c4cc5ccccc5c5ccccc45)cc3)c3cccc4sc5ccccc5c34)cc12]

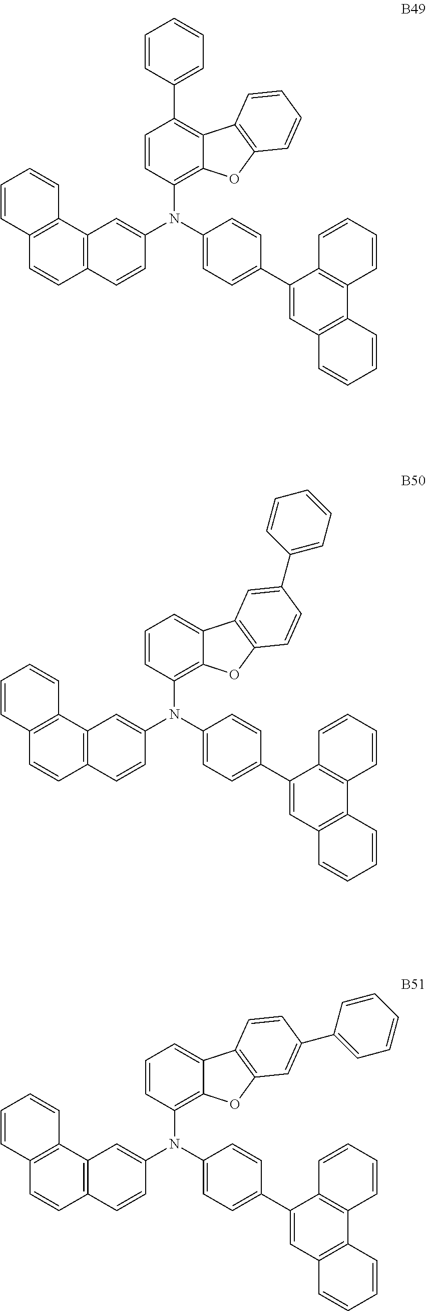 [SMILES: c1ccc(-c2ccc(N(c3ccc(-c4cc5ccccc5c5ccccc45)cc3)c3ccc4ccc5ccccc5c4c3)c3oc4ccccc4c23)cc1 ; c1ccc(-c2ccc3oc4c(N(c5ccc(-c6cc7ccccc7c7ccccc67)cc5)c5ccc6ccc7ccccc7c6c5)cccc4c3c2)cc1 ; c1ccc(-c2ccc3c(c2)oc2c(N(c4ccc(-c5cc6ccccc6c6ccccc56)cc4)c4ccc5ccc6ccccc6c5c4)cccc23)cc1]

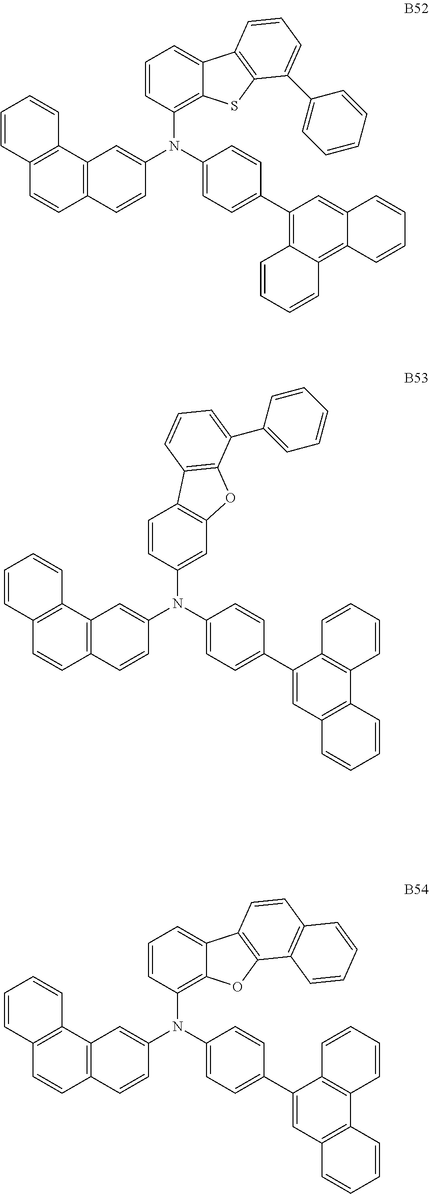 [SMILES: c1ccc(-c2cccc3c2sc2c(N(c4ccc(-c5cc6ccccc6c6ccccc56)cc4)c4ccc5ccc6ccccc6c5c4)cccc23)cc1 ; c1ccc(-c2cccc3c2oc2cc(N(c4ccc(-c5cc6ccccc6c6ccccc56)cc4)c4ccc5ccc6ccccc6c5c4)ccc23)cc1 ; c1ccc2c(c1)ccc1ccc(N(c3ccc(-c4cc5ccccc5c5ccccc45)cc3)c3cccc4c3oc3c5ccccc5ccc43)cc12]

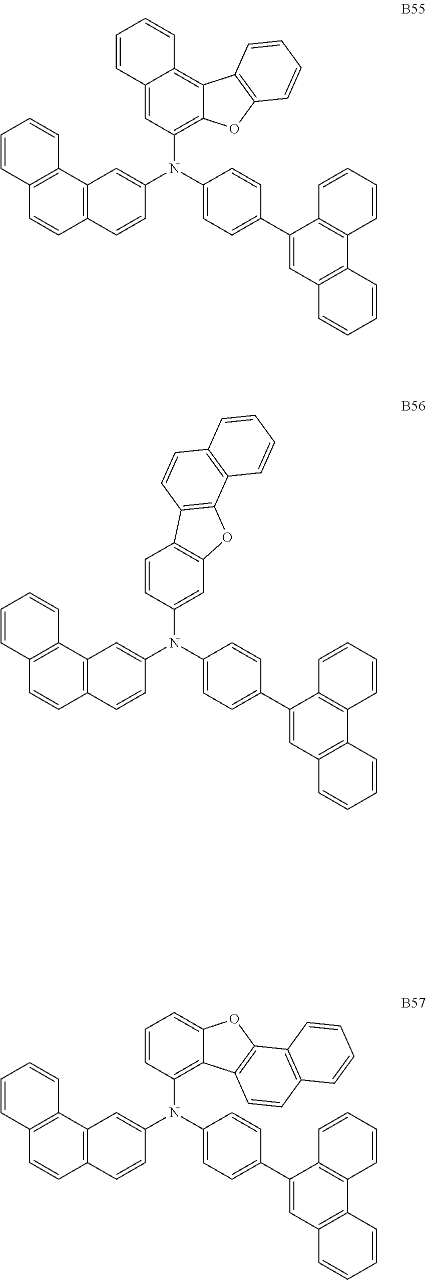 [SMILES: c1ccc2c(c1)ccc1ccc(N(c3ccc(-c4cc5ccccc5c5ccccc45)cc3)c3cc4ccccc4c4c3oc3ccccc34)cc12 ; c1ccc2c(c1)ccc1ccc(N(c3ccc(-c4cc5ccccc5c5ccccc45)cc3)c3ccc4c(c3)oc3c5ccccc5ccc43)cc12 ; c1ccc2c(c1)ccc1ccc(N(c3ccc(-c4cc5ccccc5c5ccccc45)cc3)c3cccc4oc5c6ccccc6ccc5c34)cc12]

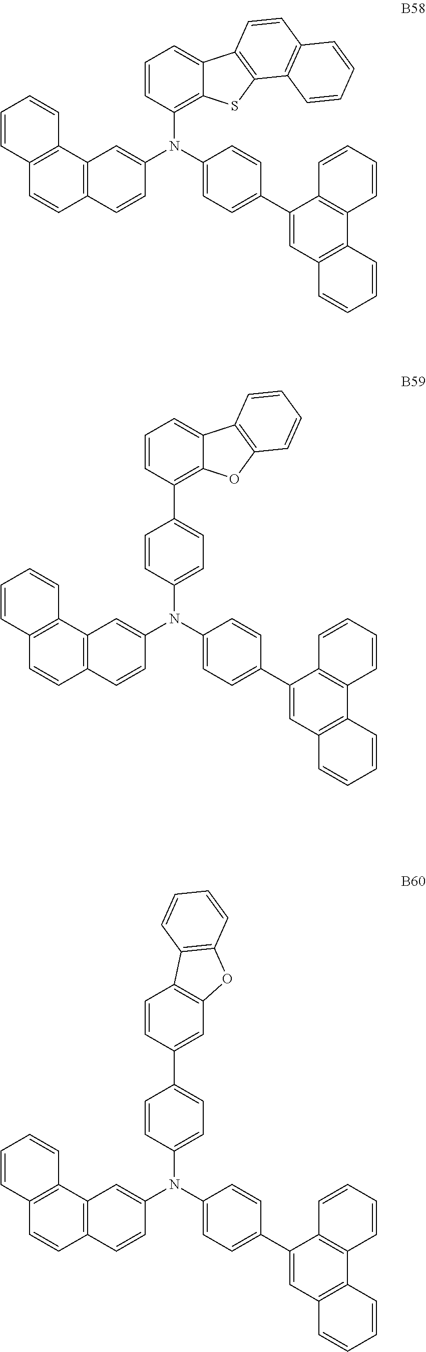 [SMILES: c1ccc2c(c1)ccc1ccc(N(c3ccc(-c4cc5ccccc5c5ccccc45)cc3)c3cccc4c3sc3c5ccccc5ccc43)cc12 ; c1ccc2c(c1)ccc1ccc(N(c3ccc(-c4cc5ccccc5c5ccccc45)cc3)c3ccc(-c4cccc5c4oc4ccccc45)cc3)cc12 ; c1ccc2c(c1)ccc1ccc(N(c3ccc(-c4ccc5c(c4)oc4ccccc45)cc3)c3ccc(-c4cc5ccccc5c5ccccc45)cc3)cc12]

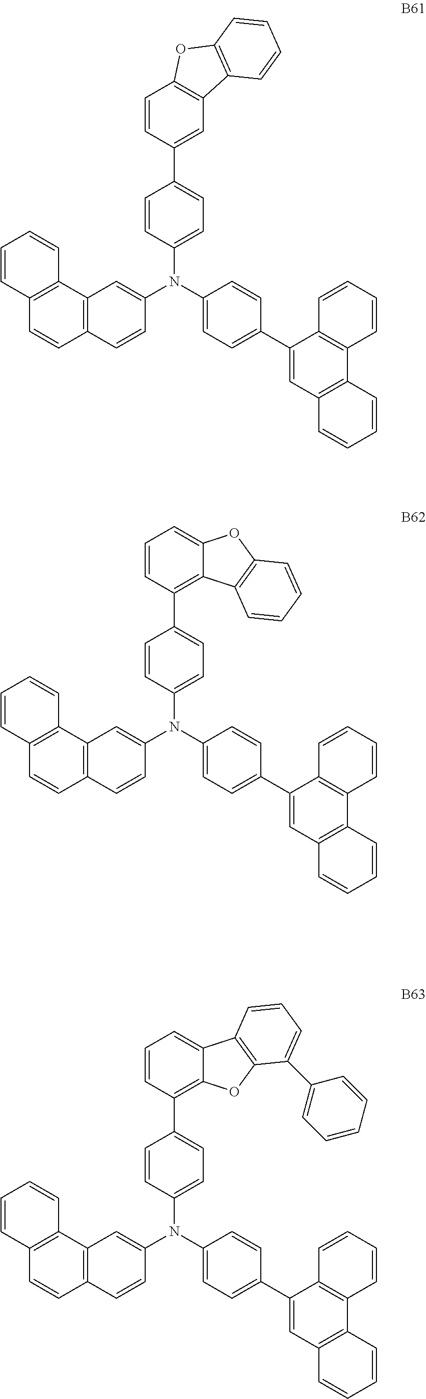 [SMILES: c1ccc2c(c1)ccc1ccc(N(c3ccc(-c4ccc5oc6ccccc6c5c4)cc3)c3ccc(-c4cc5ccccc5c5ccccc45)cc3)cc12 ; c1ccc2c(c1)ccc1ccc(N(c3ccc(-c4cc5ccccc5c5ccccc45)cc3)c3ccc(-c4cccc5oc6ccccc6c45)cc3)cc12 ; c1ccc(-c2cccc3c2oc2c(-c4ccc(N(c5ccc(-c6cc7ccccc7c7ccccc67)cc5)c5ccc6ccc7ccccc7c6c5)cc4)cccc23)cc1]

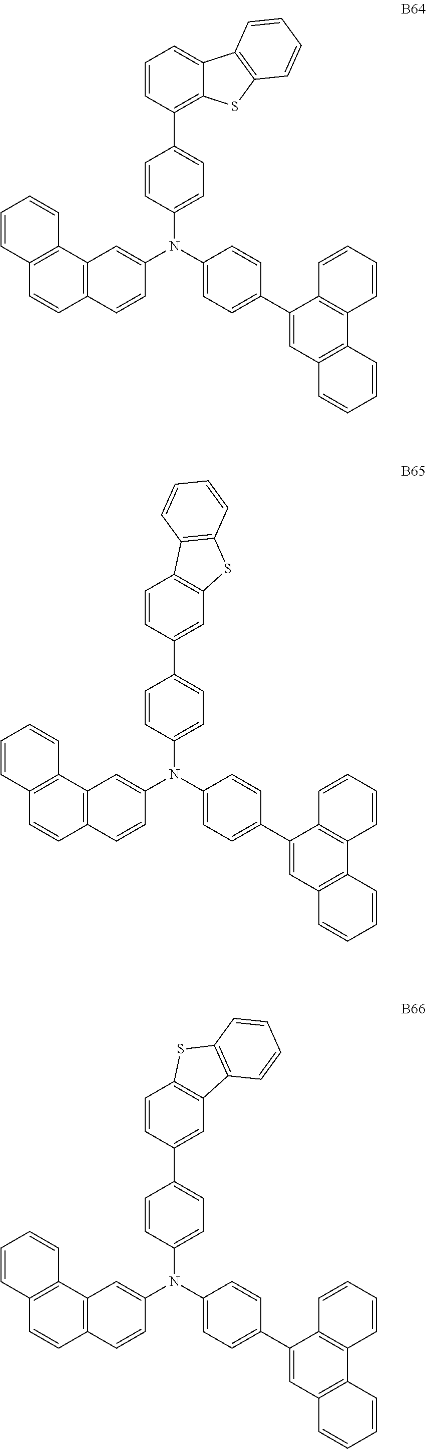 [SMILES: c1ccc2c(c1)ccc1ccc(N(c3ccc(-c4cc5ccccc5c5ccccc45)cc3)c3ccc(-c4cccc5c4sc4ccccc45)cc3)cc12 ; c1ccc2c(c1)ccc1ccc(N(c3ccc(-c4ccc5c(c4)sc4ccccc45)cc3)c3ccc(-c4cc5ccccc5c5ccccc45)cc3)cc12 ; c1ccc2c(c1)ccc1ccc(N(c3ccc(-c4ccc5sc6ccccc6c5c4)cc3)c3ccc(-c4cc5ccccc5c5ccccc45)cc3)cc12]

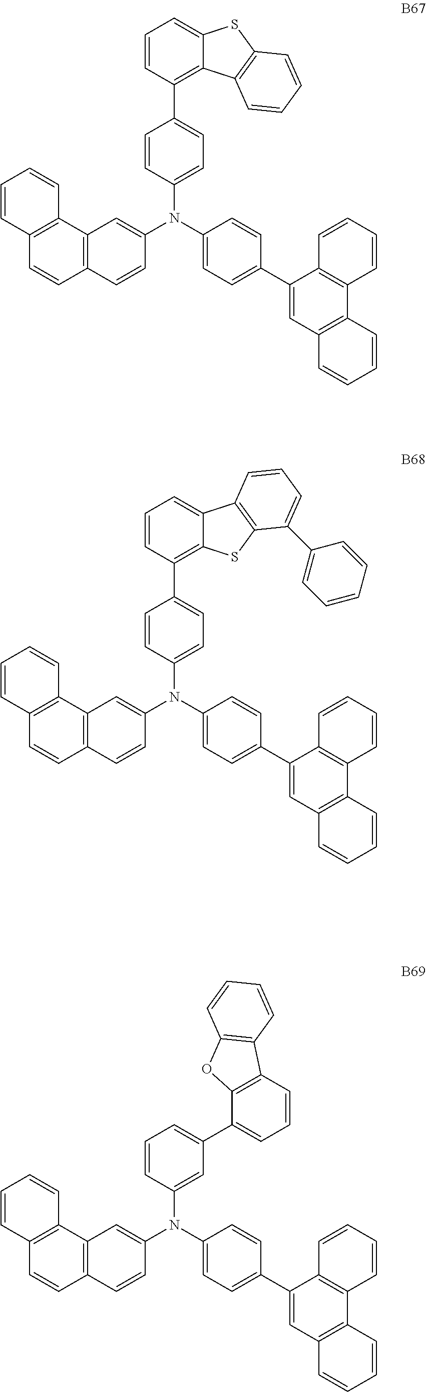 [SMILES: c1ccc2c(c1)ccc1ccc(N(c3ccc(-c4cc5ccccc5c5ccccc45)cc3)c3ccc(-c4cccc5sc6ccccc6c45)cc3)cc12 ; c1ccc(-c2cccc3c2sc2c(-c4ccc(N(c5ccc(-c6cc7ccccc7c7ccccc67)cc5)c5ccc6ccc7ccccc7c6c5)cc4)cccc23)cc1 ; c1cc(-c2cccc3c2oc2ccccc23)cc(N(c2ccc(-c3cc4ccccc4c4ccccc34)cc2)c2ccc3ccc4ccccc4c3c2)c1]

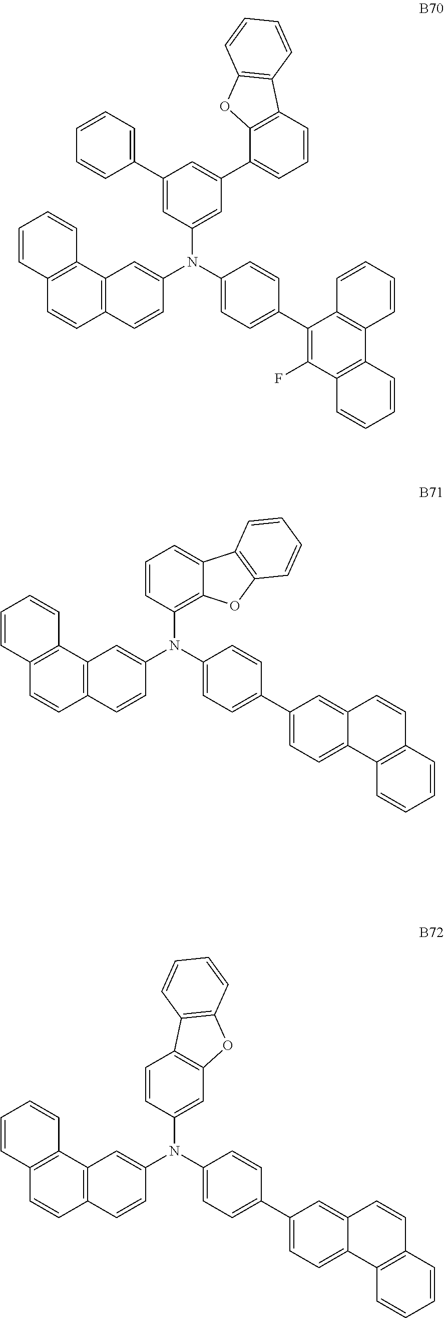 [SMILES: Fc1c(-c2ccc(N(c3cc(-c4ccccc4)cc(-c4cccc5c4oc4ccccc45)c3)c3ccc4ccc5ccccc5c4c3)cc2)c2ccccc2c2ccccc12 ; c1ccc2c(c1)ccc1cc(-c3ccc(N(c4ccc5ccc6ccccc6c5c4)c4cccc5c4oc4ccccc45)cc3)ccc12 ; c1ccc2c(c1)ccc1cc(-c3ccc(N(c4ccc5c(c4)oc4ccccc45)c4ccc5ccc6ccccc6c5c4)cc3)ccc12]

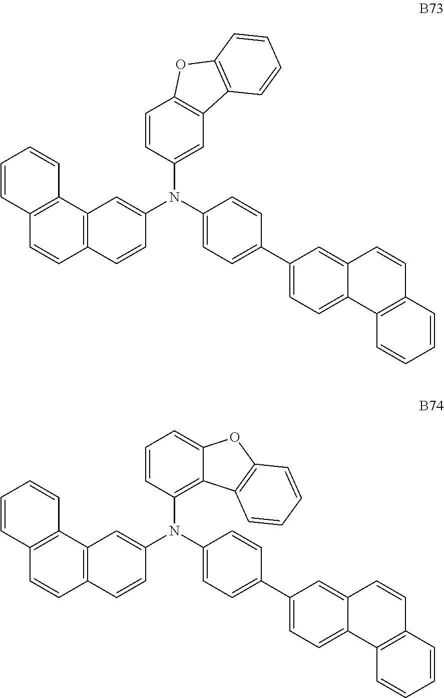 [SMILES: c1ccc2c(c1)ccc1cc(-c3ccc(N(c4ccc5ccc6ccccc6c5c4)c4ccc5oc6ccccc6c5c4)cc3)ccc12 ; c1ccc2c(c1)ccc1cc(-c3ccc(N(c4ccc5ccc6ccccc6c5c4)c4cccc5oc6ccccc6c45)cc3)ccc12]

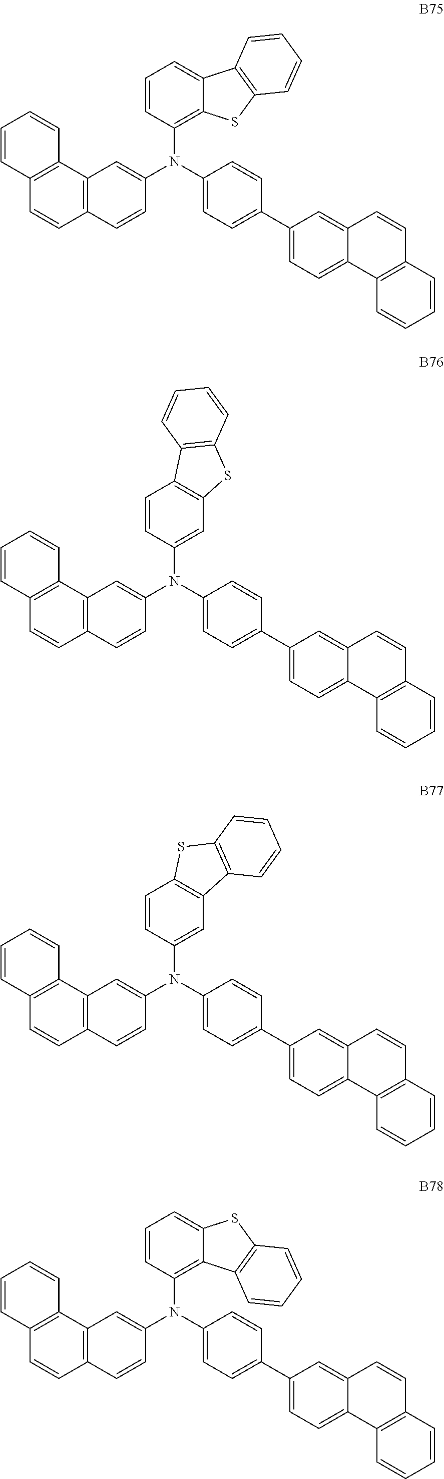 [SMILES: c1ccc2c(c1)ccc1cc(-c3ccc(N(c4ccc5ccc6ccccc6c5c4)c4cccc5c4sc4ccccc45)cc3)ccc12 ; c1ccc2c(c1)ccc1cc(-c3ccc(N(c4ccc5c(c4)sc4ccccc45)c4ccc5ccc6ccccc6c5c4)cc3)ccc12 ; c1ccc2c(c1)ccc1cc(-c3ccc(N(c4ccc5ccc6ccccc6c5c4)c4ccc5sc6ccccc6c5c4)cc3)ccc12 ; c1ccc2c(c1)ccc1cc(-c3ccc(N(c4ccc5ccc6ccccc6c5c4)c4cccc5sc6ccccc6c45)cc3)ccc12]

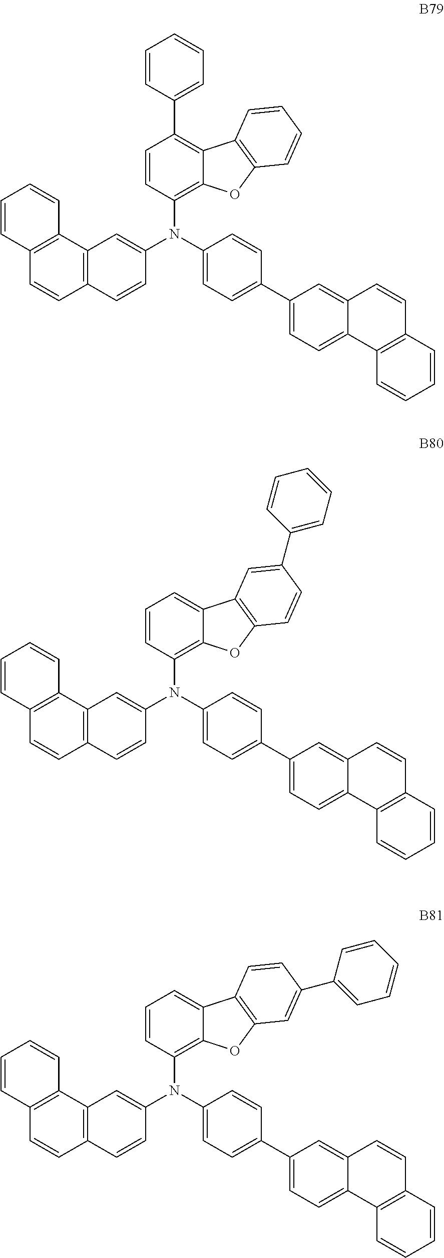 [SMILES: c1ccc(-c2ccc(N(c3ccc(-c4ccc5c(ccc6ccccc65)c4)cc3)c3ccc4ccc5ccccc5c4c3)c3oc4ccccc4c23)cc1 ; c1ccc(-c2ccc3oc4c(N(c5ccc(-c6ccc7c(ccc8ccccc87)c6)cc5)c5ccc6ccc7ccccc7c6c5)cccc4c3c2)cc1 ; c1ccc(-c2ccc3c(c2)oc2c(N(c4ccc(-c5ccc6c(ccc7ccccc76)c5)cc4)c4ccc5ccc6ccccc6c5c4)cccc23)cc1]

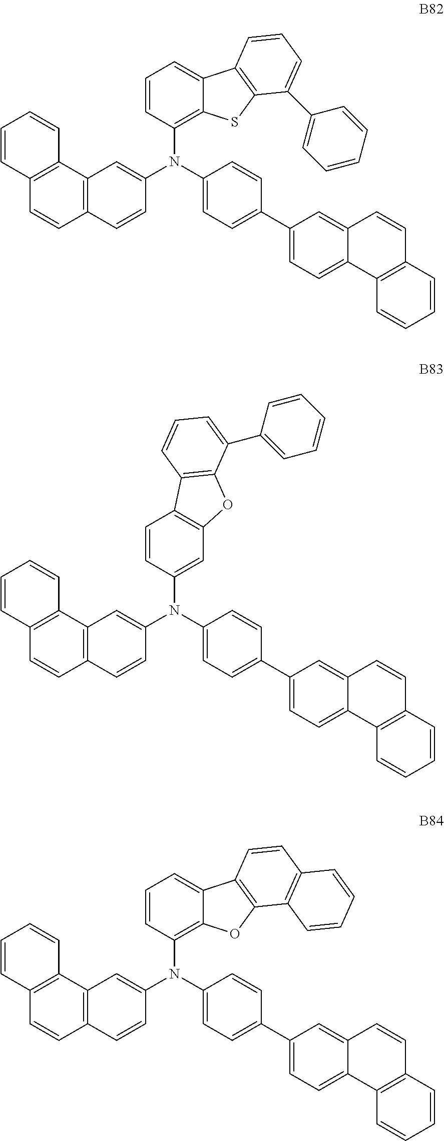 [SMILES: c1ccc(-c2cccc3c2sc2c(N(c4ccc(-c5ccc6c(ccc7ccccc76)c5)cc4)c4ccc5ccc6ccccc6c5c4)cccc23)cc1 ; c1ccc(-c2cccc3c2oc2cc(N(c4ccc(-c5ccc6c(ccc7ccccc76)c5)cc4)c4ccc5ccc6ccccc6c5c4)ccc23)cc1 ; c1ccc2c(c1)ccc1cc(-c3ccc(N(c4ccc5ccc6ccccc6c5c4)c4cccc5c4oc4c6ccccc6ccc54)cc3)ccc12]

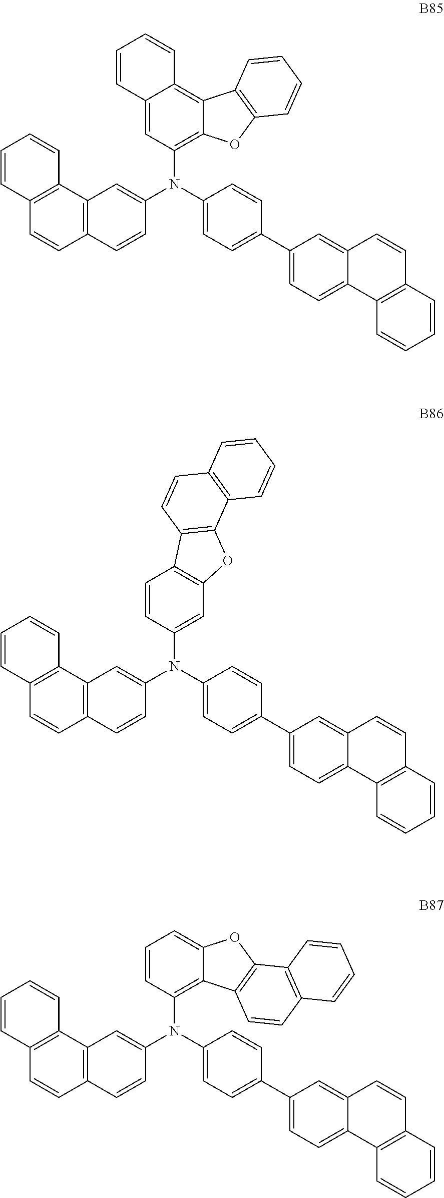 [SMILES: c1ccc2c(c1)ccc1cc(-c3ccc(N(c4ccc5ccc6ccccc6c5c4)c4cc5ccccc5c5c4oc4ccccc45)cc3)ccc12 ; c1ccc2c(c1)ccc1cc(-c3ccc(N(c4ccc5c(c4)oc4c6ccccc6ccc54)c4ccc5ccc6ccccc6c5c4)cc3)ccc12 ; c1ccc2c(c1)ccc1cc(-c3ccc(N(c4ccc5ccc6ccccc6c5c4)c4cccc5oc6c7ccccc7ccc6c45)cc3)ccc12]

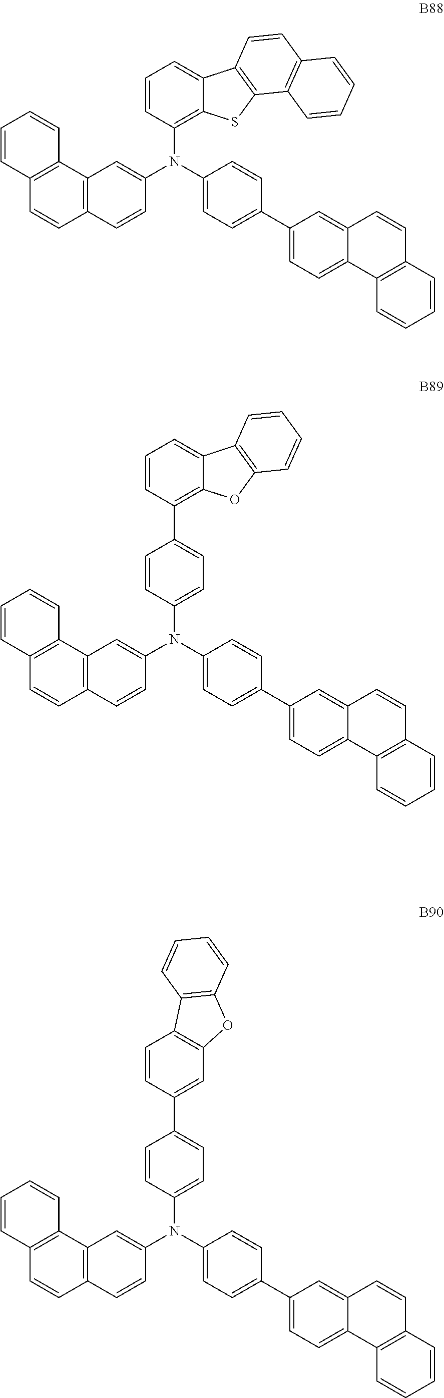 [SMILES: c1ccc2c(c1)ccc1cc(-c3ccc(N(c4ccc5ccc6ccccc6c5c4)c4cccc5c4sc4c6ccccc6ccc54)cc3)ccc12 ; c1ccc2c(c1)ccc1cc(-c3ccc(N(c4ccc(-c5cccc6c5oc5ccccc56)cc4)c4ccc5ccc6ccccc6c5c4)cc3)ccc12 ; c1ccc2c(c1)ccc1cc(-c3ccc(N(c4ccc(-c5ccc6c(c5)oc5ccccc56)cc4)c4ccc5ccc6ccccc6c5c4)cc3)ccc12]

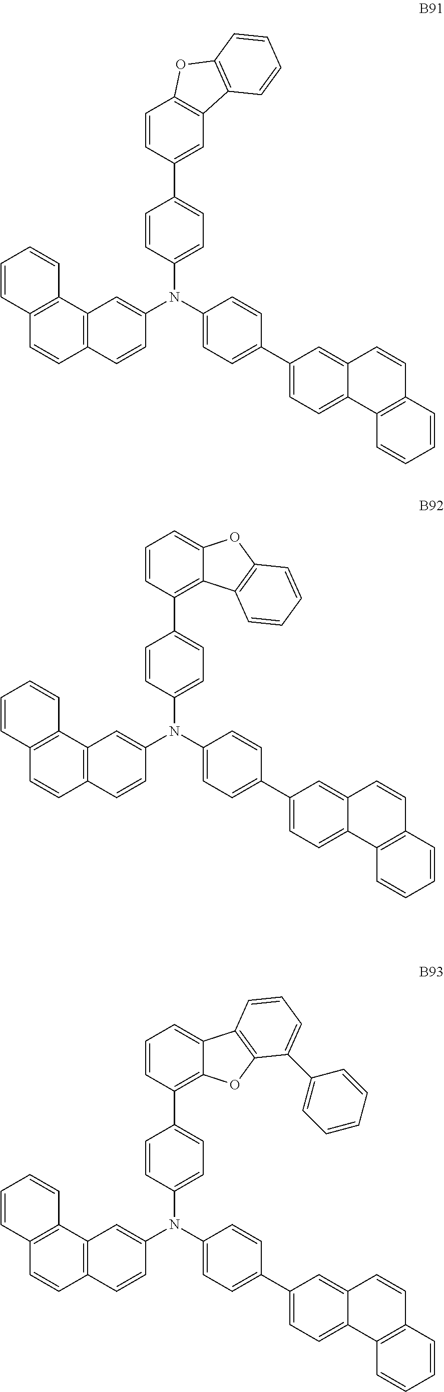 [SMILES: c1ccc2c(c1)ccc1cc(-c3ccc(N(c4ccc(-c5ccc6oc7ccccc7c6c5)cc4)c4ccc5ccc6ccccc6c5c4)cc3)ccc12 ; c1ccc2c(c1)ccc1cc(-c3ccc(N(c4ccc(-c5cccc6oc7ccccc7c56)cc4)c4ccc5ccc6ccccc6c5c4)cc3)ccc12 ; c1ccc(-c2cccc3c2oc2c(-c4ccc(N(c5ccc(-c6ccc7c(ccc8ccccc87)c6)cc5)c5ccc6ccc7ccccc7c6c5)cc4)cccc23)cc1]

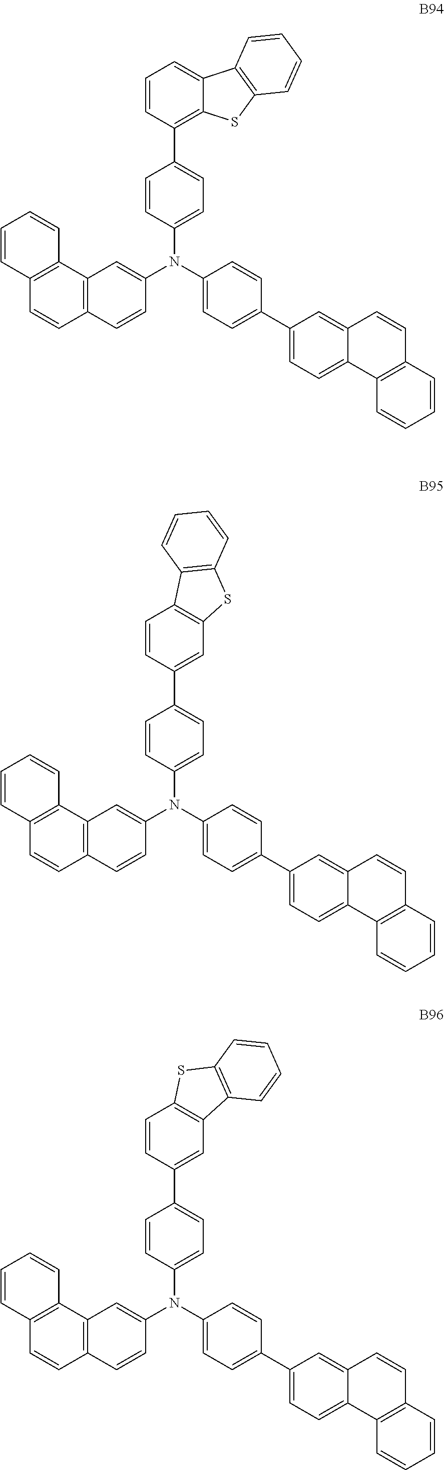 [SMILES: c1ccc2c(c1)ccc1cc(-c3ccc(N(c4ccc(-c5cccc6c5sc5ccccc56)cc4)c4ccc5ccc6ccccc6c5c4)cc3)ccc12 ; c1ccc2c(c1)ccc1cc(-c3ccc(N(c4ccc(-c5ccc6c(c5)sc5ccccc56)cc4)c4ccc5ccc6ccccc6c5c4)cc3)ccc12 ; c1ccc2c(c1)ccc1cc(-c3ccc(N(c4ccc(-c5ccc6sc7ccccc7c6c5)cc4)c4ccc5ccc6ccccc6c5c4)cc3)ccc12]

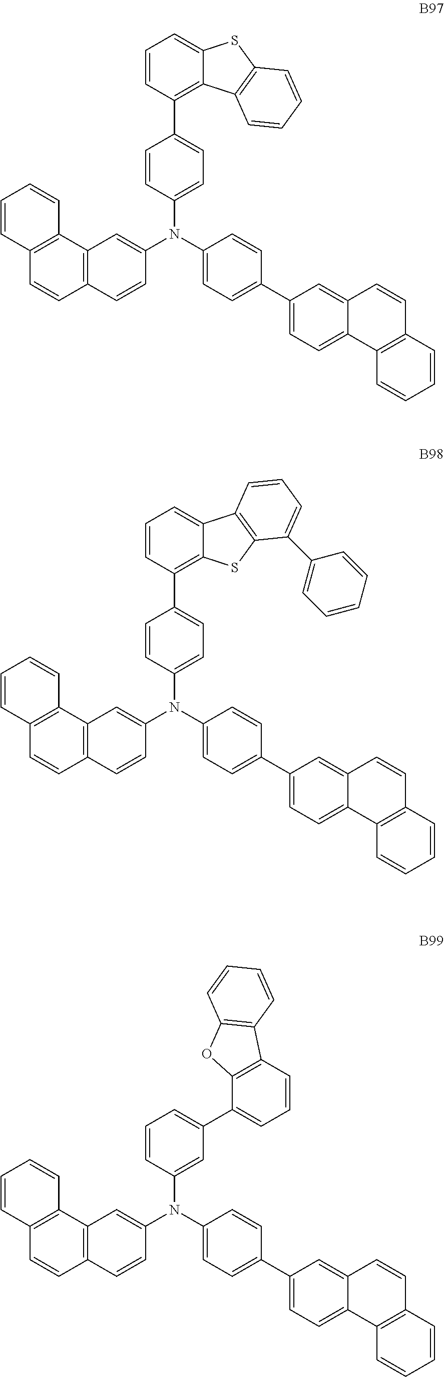 [SMILES: c1ccc2c(c1)ccc1cc(-c3ccc(N(c4ccc(-c5cccc6sc7ccccc7c56)cc4)c4ccc5ccc6ccccc6c5c4)cc3)ccc12 ; c1ccc(-c2cccc3c2sc2c(-c4ccc(N(c5ccc(-c6ccc7c(ccc8ccccc87)c6)cc5)c5ccc6ccc7ccccc7c6c5)cc4)cccc23)cc1 ; c1cc(-c2cccc3c2oc2ccccc23)cc(N(c2ccc(-c3ccc4c(ccc5ccccc54)c3)cc2)c2ccc3ccc4ccccc4c3c2)c1]

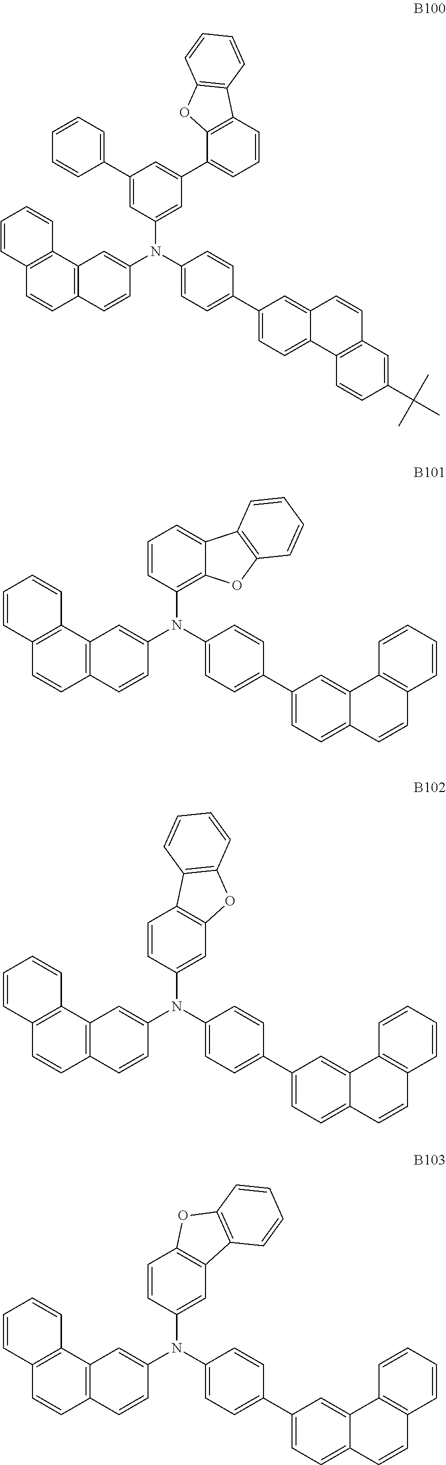 [SMILES: CC(C)(C)c1ccc2c(ccc3cc(-c4ccc(N(c5cc(-c6ccccc6)cc(-c6cccc7c6oc6ccccc67)c5)c5ccc6ccc7ccccc7c6c5)cc4)ccc32)c1 ; c1ccc2c(c1)ccc1ccc(-c3ccc(N(c4ccc5ccc6ccccc6c5c4)c4cccc5c4oc4ccccc45)cc3)cc12 ; c1ccc2c(c1)ccc1ccc(-c3ccc(N(c4ccc5c(c4)oc4ccccc45)c4ccc5ccc6ccccc6c5c4)cc3)cc12 ; c1ccc2c(c1)ccc1ccc(-c3ccc(N(c4ccc5ccc6ccccc6c5c4)c4ccc5oc6ccccc6c5c4)cc3)cc12]

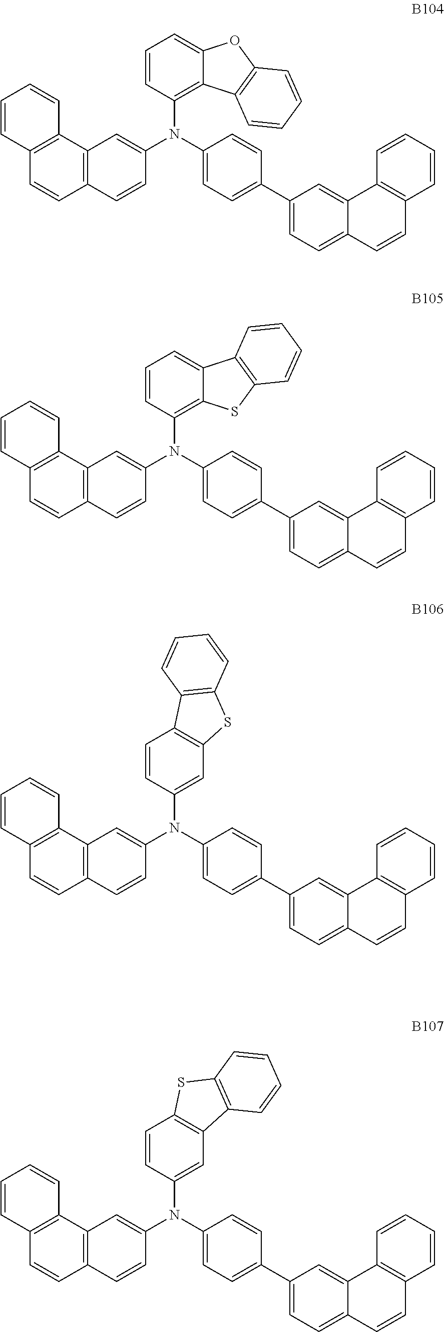 [SMILES: c1ccc2c(c1)ccc1ccc(-c3ccc(N(c4ccc5ccc6ccccc6c5c4)c4cccc5oc6ccccc6c45)cc3)cc12 ; c1ccc2c(c1)ccc1ccc(-c3ccc(N(c4ccc5ccc6ccccc6c5c4)c4cccc5c4sc4ccccc45)cc3)cc12 ; c1ccc2c(c1)ccc1ccc(-c3ccc(N(c4ccc5c(c4)sc4ccccc45)c4ccc5ccc6ccccc6c5c4)cc3)cc12 ; c1ccc2c(c1)ccc1ccc(-c3ccc(N(c4ccc5ccc6ccccc6c5c4)c4ccc5sc6ccccc6c5c4)cc3)cc12]

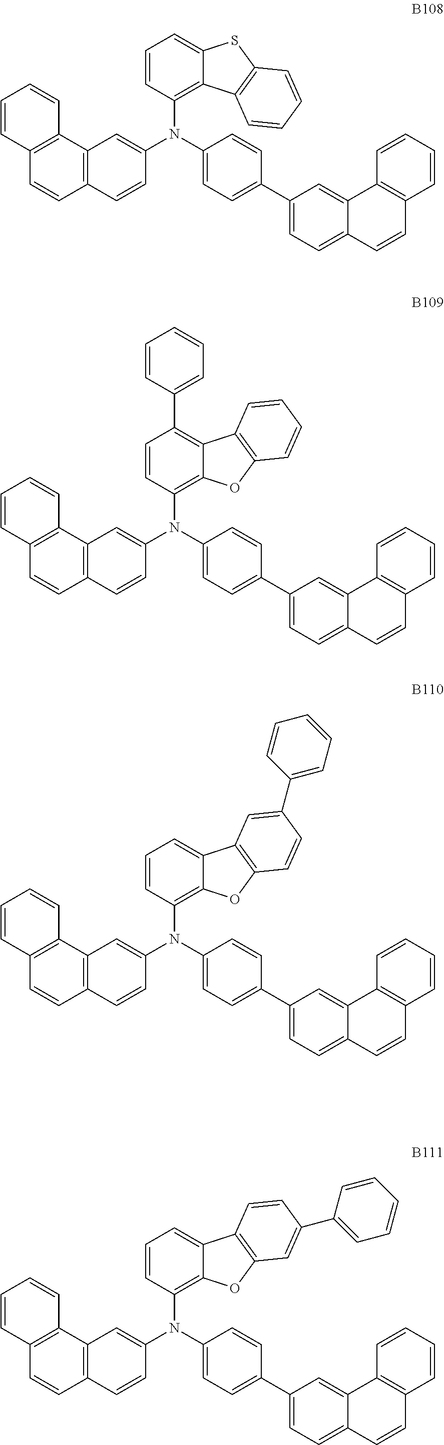 [SMILES: c1ccc2c(c1)ccc1ccc(-c3ccc(N(c4ccc5ccc6ccccc6c5c4)c4cccc5sc6ccccc6c45)cc3)cc12 ; c1ccc(-c2ccc(N(c3ccc(-c4ccc5ccc6ccccc6c5c4)cc3)c3ccc4ccc5ccccc5c4c3)c3oc4ccccc4c23)cc1 ; c1ccc(-c2ccc3oc4c(N(c5ccc(-c6ccc7ccc8ccccc8c7c6)cc5)c5ccc6ccc7ccccc7c6c5)cccc4c3c2)cc1 ; c1ccc(-c2ccc3c(c2)oc2c(N(c4ccc(-c5ccc6ccc7ccccc7c6c5)cc4)c4ccc5ccc6ccccc6c5c4)cccc23)cc1]

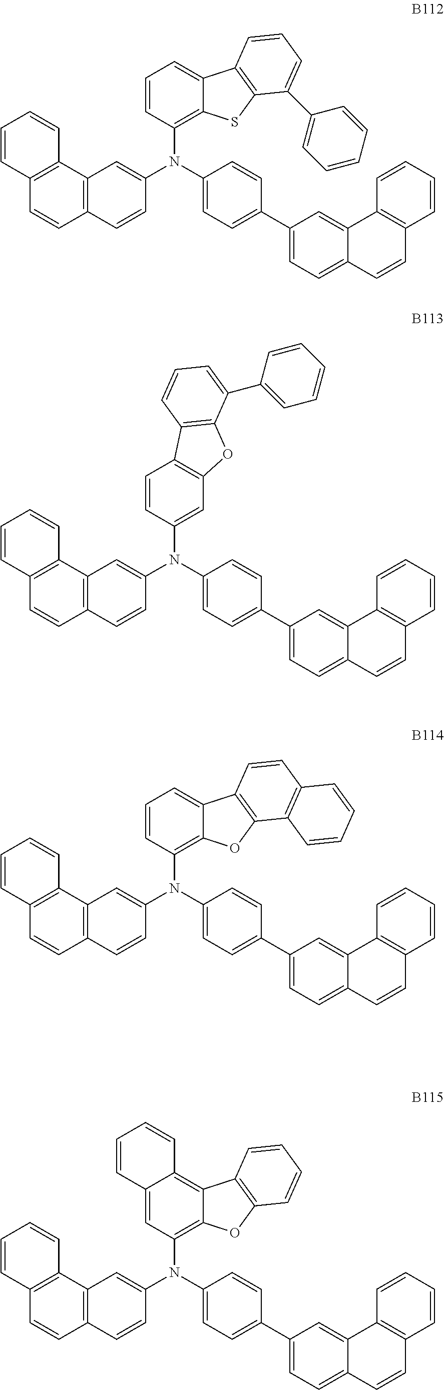 [SMILES: c1ccc(-c2cccc3c2sc2c(N(c4ccc(-c5ccc6ccc7ccccc7c6c5)cc4)c4ccc5ccc6ccccc6c5c4)cccc23)cc1 ; c1ccc(-c2cccc3c2oc2cc(N(c4ccc(-c5ccc6ccc7ccccc7c6c5)cc4)c4ccc5ccc6ccccc6c5c4)ccc23)cc1 ; c1ccc2c(c1)ccc1ccc(-c3ccc(N(c4ccc5ccc6ccccc6c5c4)c4cccc5c4oc4c6ccccc6ccc54)cc3)cc12 ; c1ccc2c(c1)ccc1ccc(-c3ccc(N(c4ccc5ccc6ccccc6c5c4)c4cc5ccccc5c5c4oc4ccccc45)cc3)cc12]

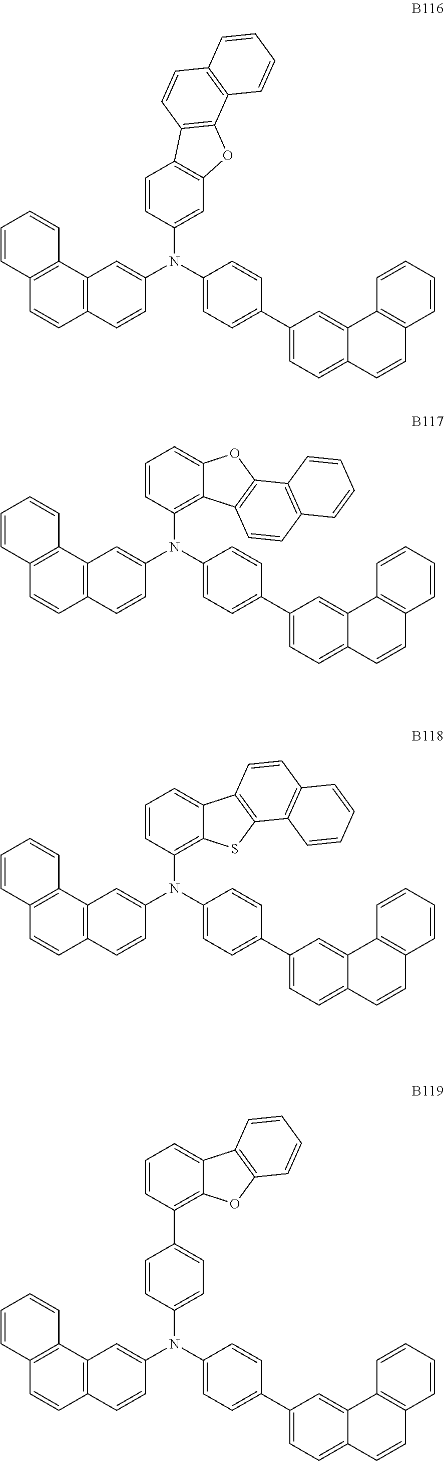 [SMILES: c1ccc2c(c1)ccc1ccc(-c3ccc(N(c4ccc5c(c4)oc4c6ccccc6ccc54)c4ccc5ccc6ccccc6c5c4)cc3)cc12 ; c1ccc2c(c1)ccc1ccc(-c3ccc(N(c4ccc5ccc6ccccc6c5c4)c4cccc5oc6c7ccccc7ccc6c45)cc3)cc12 ; c1ccc2c(c1)ccc1ccc(-c3ccc(N(c4ccc5ccc6ccccc6c5c4)c4cccc5c4sc4c6ccccc6ccc54)cc3)cc12 ; c1ccc2c(c1)ccc1ccc(-c3ccc(N(c4ccc(-c5cccc6c5oc5ccccc56)cc4)c4ccc5ccc6ccccc6c5c4)cc3)cc12]

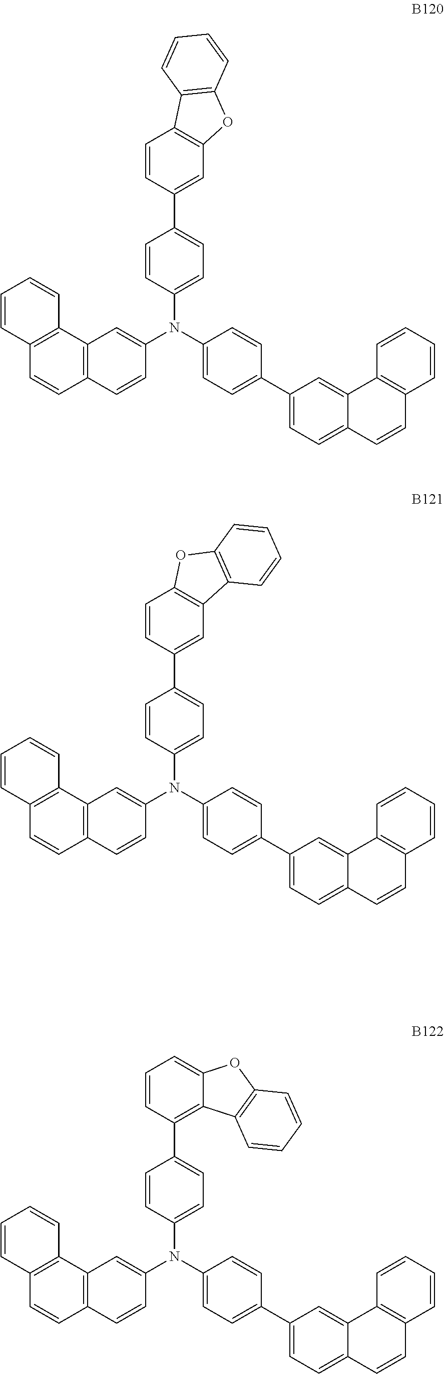 [SMILES: c1ccc2c(c1)ccc1ccc(-c3ccc(N(c4ccc(-c5ccc6c(c5)oc5ccccc56)cc4)c4ccc5ccc6ccccc6c5c4)cc3)cc12 ; c1ccc2c(c1)ccc1ccc(-c3ccc(N(c4ccc(-c5ccc6oc7ccccc7c6c5)cc4)c4ccc5ccc6ccccc6c5c4)cc3)cc12 ; c1ccc2c(c1)ccc1ccc(-c3ccc(N(c4ccc(-c5cccc6oc7ccccc7c56)cc4)c4ccc5ccc6ccccc6c5c4)cc3)cc12]

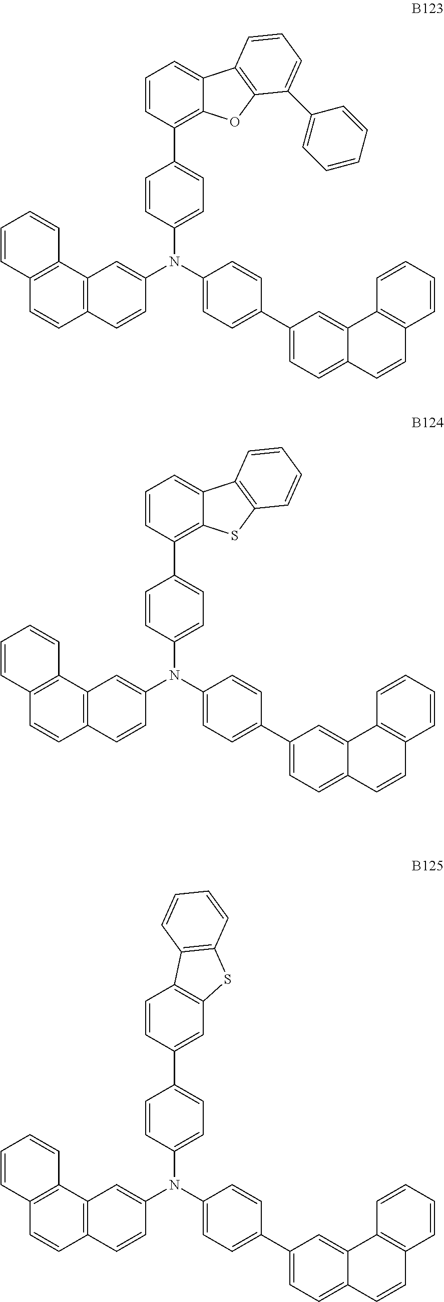 [SMILES: c1ccc(-c2cccc3c2oc2c(-c4ccc(N(c5ccc(-c6ccc7ccc8ccccc8c7c6)cc5)c5ccc6ccc7ccccc7c6c5)cc4)cccc23)cc1 ; c1ccc2c(c1)ccc1ccc(-c3ccc(N(c4ccc(-c5cccc6c5sc5ccccc56)cc4)c4ccc5ccc6ccccc6c5c4)cc3)cc12 ; c1ccc2c(c1)ccc1ccc(-c3ccc(N(c4ccc(-c5ccc6c(c5)sc5ccccc56)cc4)c4ccc5ccc6ccccc6c5c4)cc3)cc12]

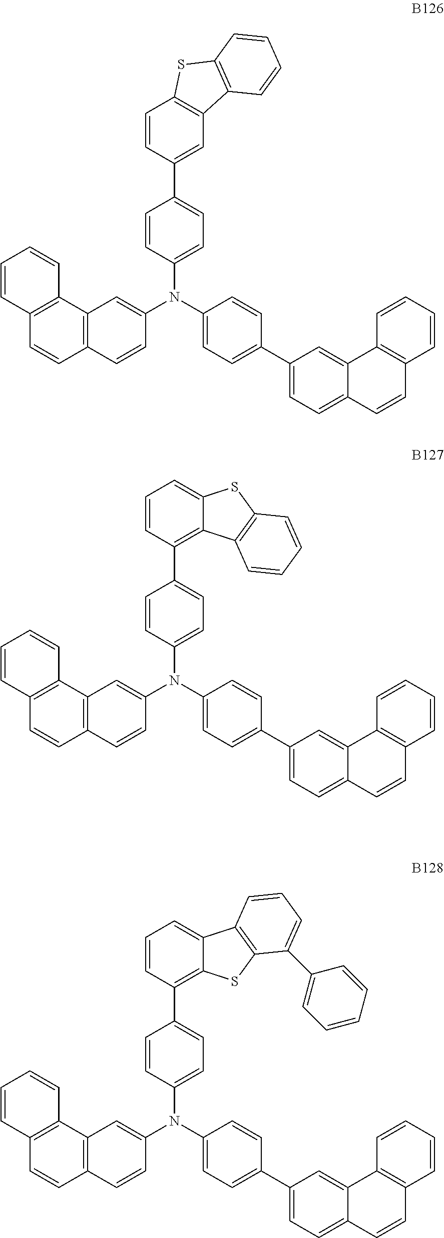 [SMILES: c1ccc2c(c1)ccc1ccc(-c3ccc(N(c4ccc(-c5ccc6sc7ccccc7c6c5)cc4)c4ccc5ccc6ccccc6c5c4)cc3)cc12 ; c1ccc2c(c1)ccc1ccc(-c3ccc(N(c4ccc(-c5cccc6sc7ccccc7c56)cc4)c4ccc5ccc6ccccc6c5c4)cc3)cc12 ; c1ccc(-c2cccc3c2sc2c(-c4ccc(N(c5ccc(-c6ccc7ccc8ccccc8c7c6)cc5)c5ccc6ccc7ccccc7c6c5)cc4)cccc23)cc1]

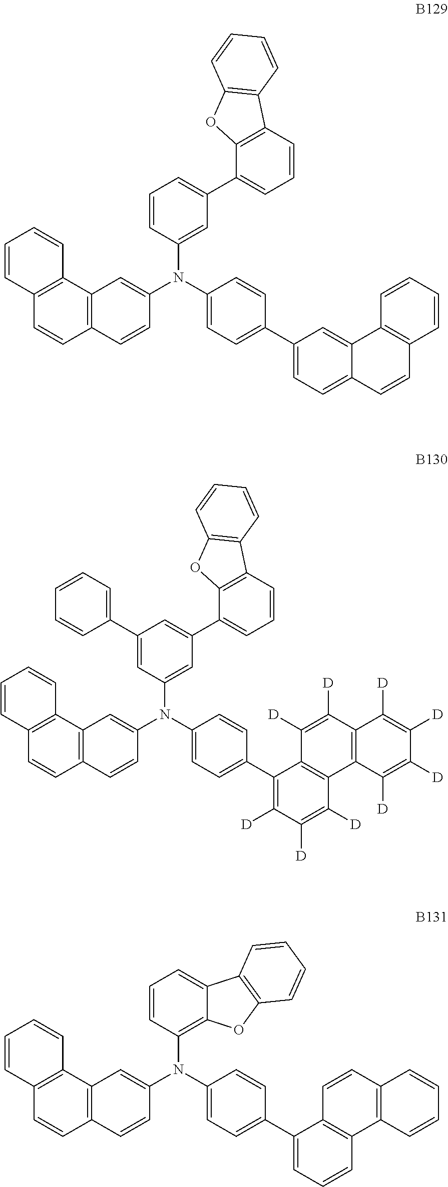 [SMILES: c1cc(-c2cccc3c2oc2ccccc23)cc(N(c2ccc(-c3ccc4ccc5ccccc5c4c3)cc2)c2ccc3ccc4ccccc4c3c2)c1 ; [2H]c1c([2H])c([2H])c2c(c1[2H])c([2H])c([2H])c1c(-c3ccc(N(c4cc(-c5ccccc5)cc(-c5cccc6c5oc5ccccc56)c4)c4ccc5ccc6ccccc6c5c4)cc3)c([2H])c([2H])c([2H])c12 ; c1ccc2c(c1)ccc1ccc(N(c3ccc(-c4cccc5c4ccc4ccccc45)cc3)c3cccc4c3oc3ccccc34)cc12]

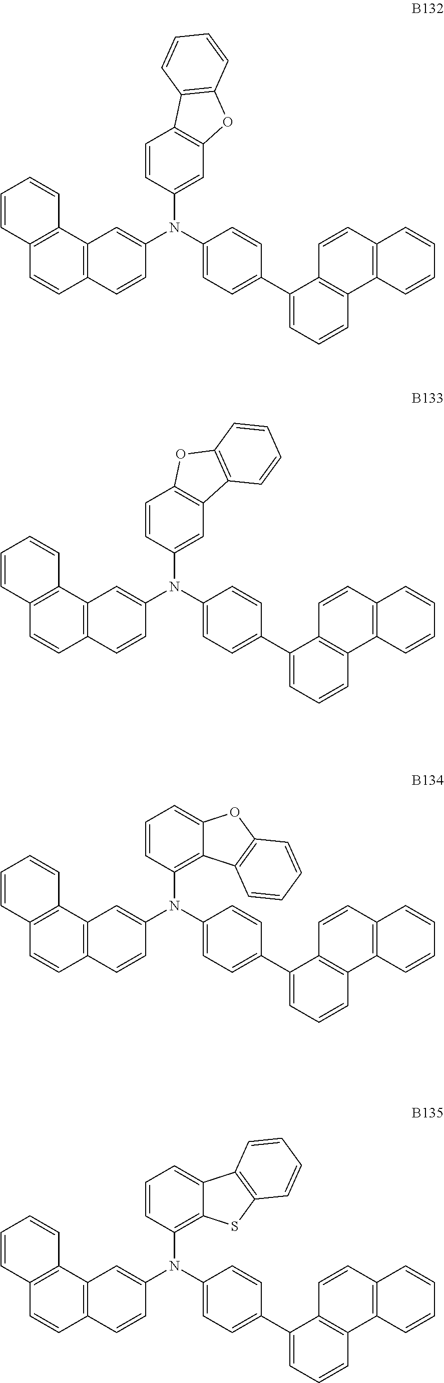 [SMILES: c1ccc2c(c1)ccc1ccc(N(c3ccc(-c4cccc5c4ccc4ccccc45)cc3)c3ccc4c(c3)oc3ccccc34)cc12 ; c1ccc2c(c1)ccc1ccc(N(c3ccc(-c4cccc5c4ccc4ccccc45)cc3)c3ccc4oc5ccccc5c4c3)cc12 ; c1ccc2c(c1)ccc1ccc(N(c3ccc(-c4cccc5c4ccc4ccccc45)cc3)c3cccc4oc5ccccc5c34)cc12 ; c1ccc2c(c1)ccc1ccc(N(c3ccc(-c4cccc5c4ccc4ccccc45)cc3)c3cccc4c3sc3ccccc34)cc12]

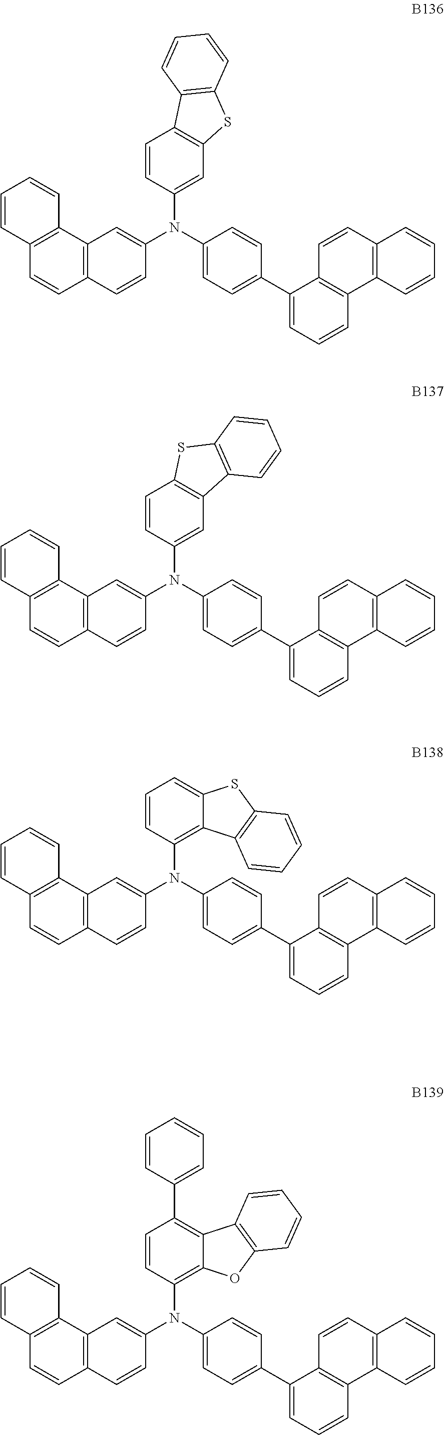 [SMILES: c1ccc2c(c1)ccc1ccc(N(c3ccc(-c4cccc5c4ccc4ccccc45)cc3)c3ccc4c(c3)sc3ccccc34)cc12 ; c1ccc2c(c1)ccc1ccc(N(c3ccc(-c4cccc5c4ccc4ccccc45)cc3)c3ccc4sc5ccccc5c4c3)cc12 ; c1ccc2c(c1)ccc1ccc(N(c3ccc(-c4cccc5c4ccc4ccccc45)cc3)c3cccc4sc5ccccc5c34)cc12 ; c1ccc(-c2ccc(N(c3ccc(-c4cccc5c4ccc4ccccc45)cc3)c3ccc4ccc5ccccc5c4c3)c3oc4ccccc4c23)cc1]

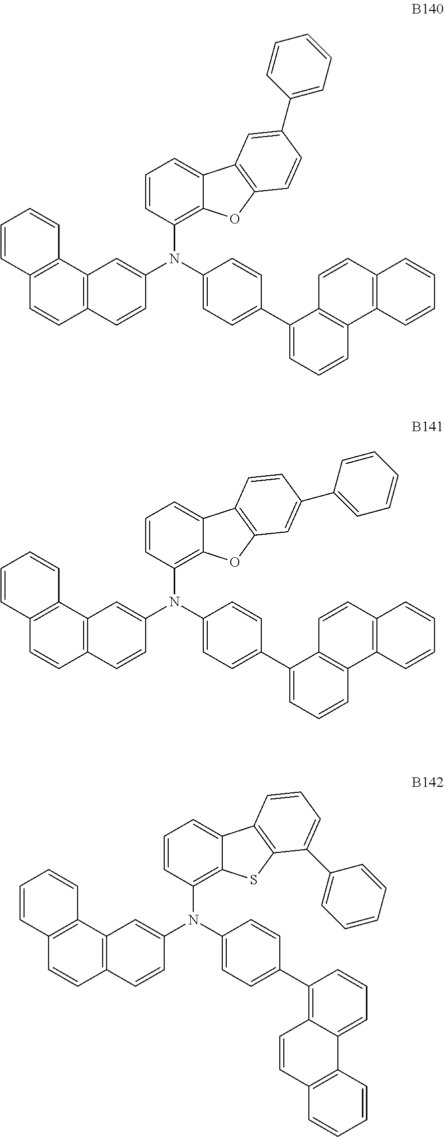 [SMILES: c1ccc(-c2ccc3oc4c(N(c5ccc(-c6cccc7c6ccc6ccccc67)cc5)c5ccc6ccc7ccccc7c6c5)cccc4c3c2)cc1 ; c1ccc(-c2ccc3c(c2)oc2c(N(c4ccc(-c5cccc6c5ccc5ccccc56)cc4)c4ccc5ccc6ccccc6c5c4)cccc23)cc1 ; c1ccc(-c2cccc3c2sc2c(N(c4ccc(-c5cccc6c5ccc5ccccc56)cc4)c4ccc5ccc6ccccc6c5c4)cccc23)cc1]

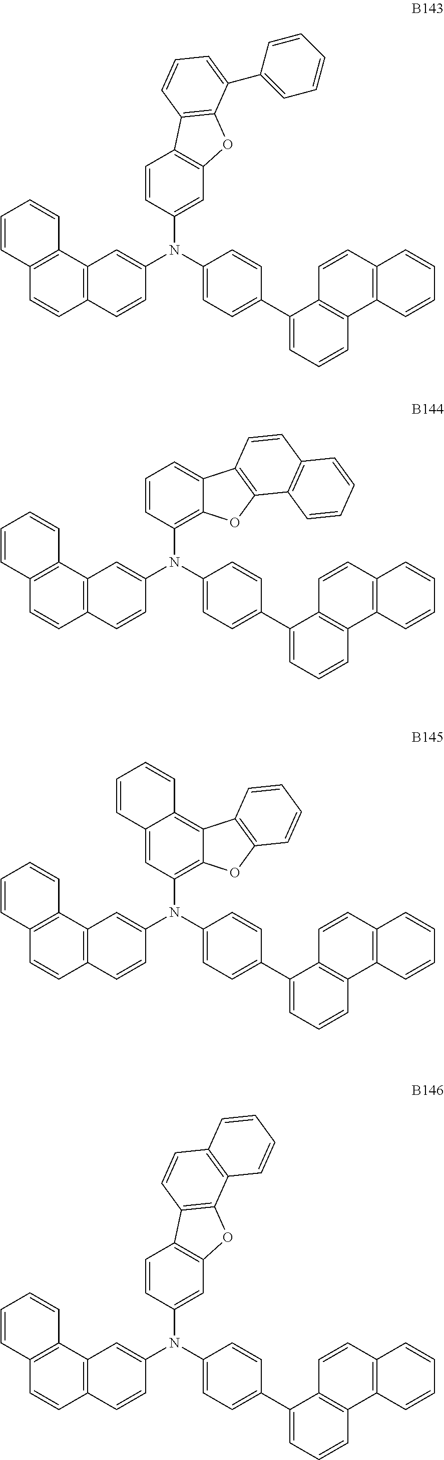 [SMILES: c1ccc(-c2cccc3c2oc2cc(N(c4ccc(-c5cccc6c5ccc5ccccc56)cc4)c4ccc5ccc6ccccc6c5c4)ccc23)cc1 ; c1ccc2c(c1)ccc1ccc(N(c3ccc(-c4cccc5c4ccc4ccccc45)cc3)c3cccc4c3oc3c5ccccc5ccc43)cc12 ; c1ccc2c(c1)ccc1ccc(N(c3ccc(-c4cccc5c4ccc4ccccc45)cc3)c3cc4ccccc4c4c3oc3ccccc34)cc12 ; c1ccc2c(c1)ccc1ccc(N(c3ccc(-c4cccc5c4ccc4ccccc45)cc3)c3ccc4c(c3)oc3c5ccccc5ccc43)cc12]

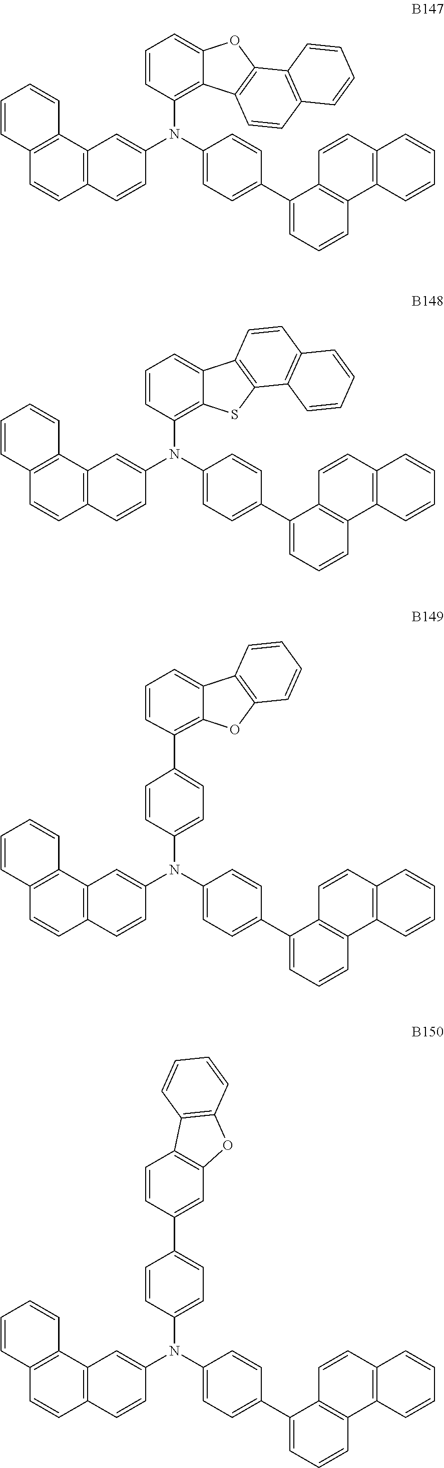 [SMILES: c1ccc2c(c1)ccc1ccc(N(c3ccc(-c4cccc5c4ccc4ccccc45)cc3)c3cccc4oc5c6ccccc6ccc5c34)cc12 ; c1ccc2c(c1)ccc1ccc(N(c3ccc(-c4cccc5c4ccc4ccccc45)cc3)c3cccc4c3sc3c5ccccc5ccc43)cc12 ; c1ccc2c(c1)ccc1ccc(N(c3ccc(-c4cccc5c4ccc4ccccc45)cc3)c3ccc(-c4cccc5c4oc4ccccc45)cc3)cc12 ; c1ccc2c(c1)ccc1ccc(N(c3ccc(-c4ccc5c(c4)oc4ccccc45)cc3)c3ccc(-c4cccc5c4ccc4ccccc45)cc3)cc12]

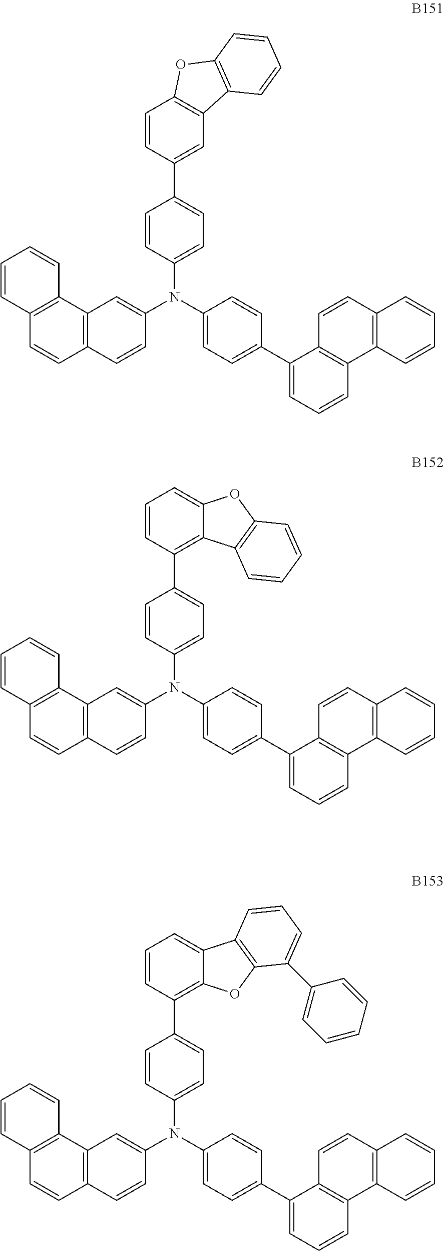 [SMILES: c1ccc2c(c1)ccc1ccc(N(c3ccc(-c4ccc5oc6ccccc6c5c4)cc3)c3ccc(-c4cccc5c4ccc4ccccc45)cc3)cc12 ; c1ccc2c(c1)ccc1ccc(N(c3ccc(-c4cccc5c4ccc4ccccc45)cc3)c3ccc(-c4cccc5oc6ccccc6c45)cc3)cc12 ; c1ccc(-c2cccc3c2oc2c(-c4ccc(N(c5ccc(-c6cccc7c6ccc6ccccc67)cc5)c5ccc6ccc7ccccc7c6c5)cc4)cccc23)cc1]

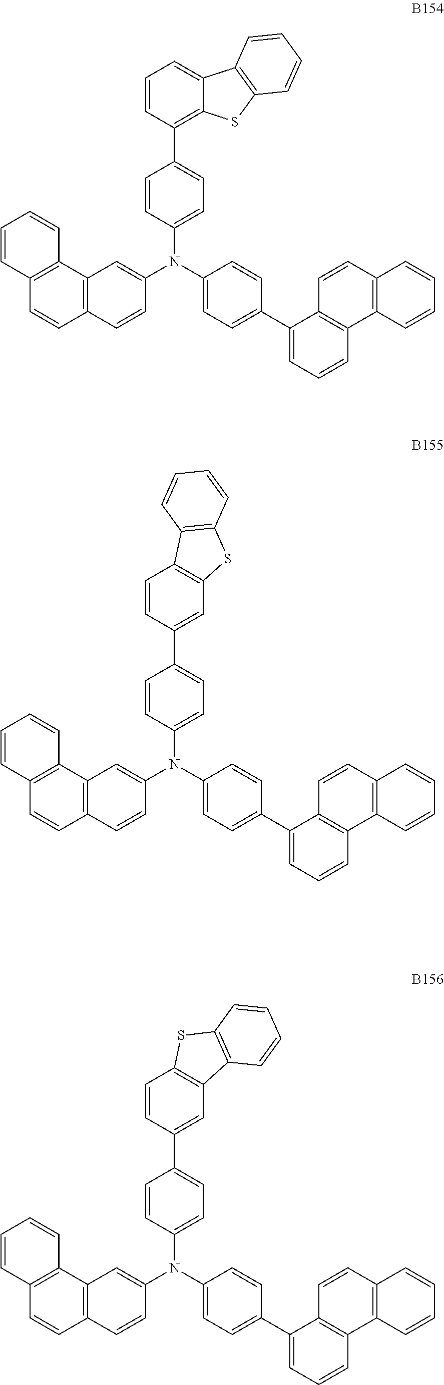 [SMILES: c1ccc2c(c1)ccc1ccc(N(c3ccc(-c4cccc5c4ccc4ccccc45)cc3)c3ccc(-c4cccc5c4sc4ccccc45)cc3)cc12 ; c1ccc2c(c1)ccc1ccc(N(c3ccc(-c4ccc5c(c4)sc4ccccc45)cc3)c3ccc(-c4cccc5c4ccc4ccccc45)cc3)cc12 ; c1ccc2c(c1)ccc1ccc(N(c3ccc(-c4ccc5sc6ccccc6c5c4)cc3)c3ccc(-c4cccc5c4ccc4ccccc45)cc3)cc12]

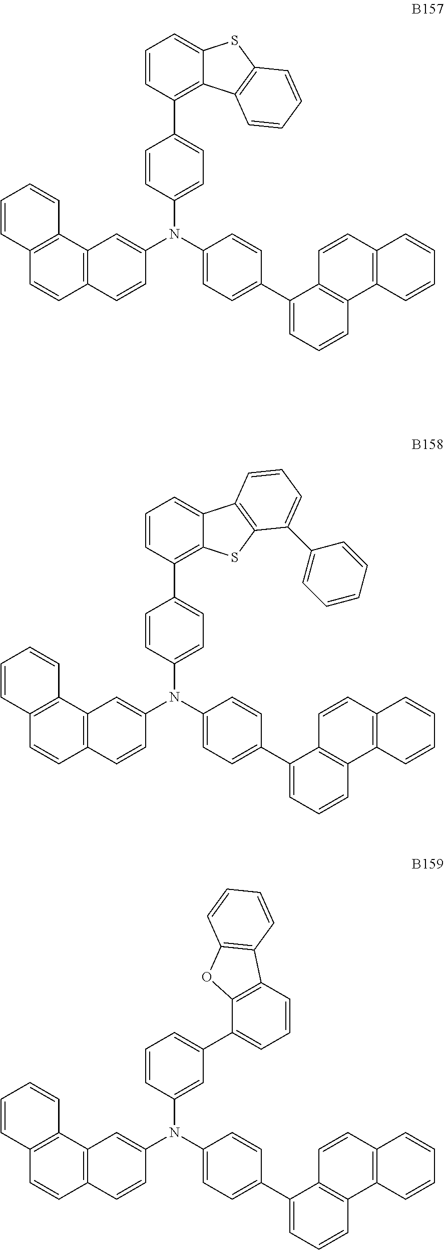 [SMILES: c1ccc2c(c1)ccc1ccc(N(c3ccc(-c4cccc5c4ccc4ccccc45)cc3)c3ccc(-c4cccc5sc6ccccc6c45)cc3)cc12 ; c1ccc(-c2cccc3c2sc2c(-c4ccc(N(c5ccc(-c6cccc7c6ccc6ccccc67)cc5)c5ccc6ccc7ccccc7c6c5)cc4)cccc23)cc1 ; c1cc(-c2cccc3c2oc2ccccc23)cc(N(c2ccc(-c3cccc4c3ccc3ccccc34)cc2)c2ccc3ccc4ccccc4c3c2)c1]

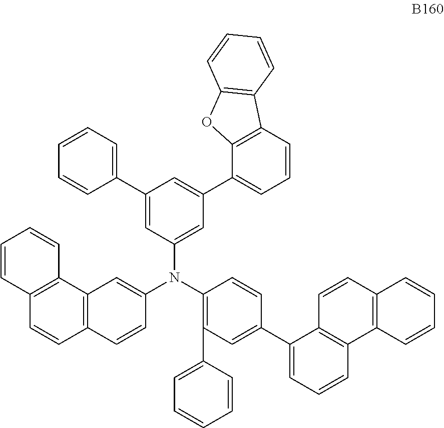 [SMILES: c1ccc(-c2cc(-c3cccc4c3oc3ccccc34)cc(N(c3ccc4ccc5ccccc5c4c3)c3ccc(-c4cccc5c4ccc4ccccc45)cc3-c3ccccc3)c2)cc1]

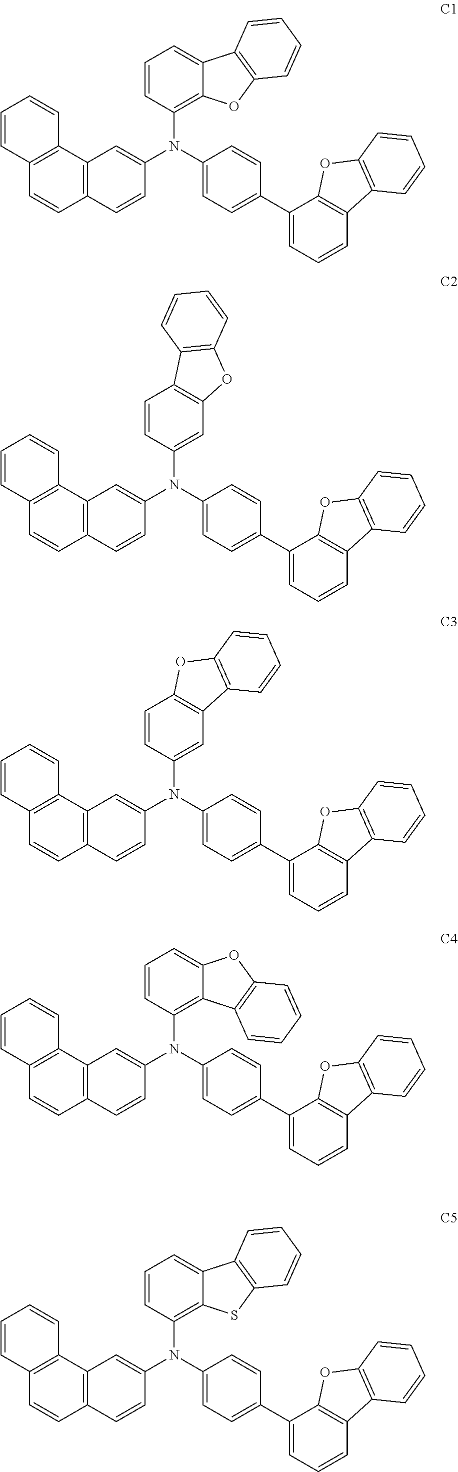 [SMILES: c1ccc2c(c1)ccc1ccc(N(c3ccc(-c4cccc5c4oc4ccccc45)cc3)c3cccc4c3oc3ccccc34)cc12 ; c1ccc2c(c1)ccc1ccc(N(c3ccc(-c4cccc5c4oc4ccccc45)cc3)c3ccc4c(c3)oc3ccccc34)cc12 ; c1ccc2c(c1)ccc1ccc(N(c3ccc(-c4cccc5c4oc4ccccc45)cc3)c3ccc4oc5ccccc5c4c3)cc12 ; c1ccc2c(c1)ccc1ccc(N(c3ccc(-c4cccc5c4oc4ccccc45)cc3)c3cccc4oc5ccccc5c34)cc12 ; c1ccc2c(c1)ccc1ccc(N(c3ccc(-c4cccc5c4oc4ccccc45)cc3)c3cccc4c3sc3ccccc34)cc12]

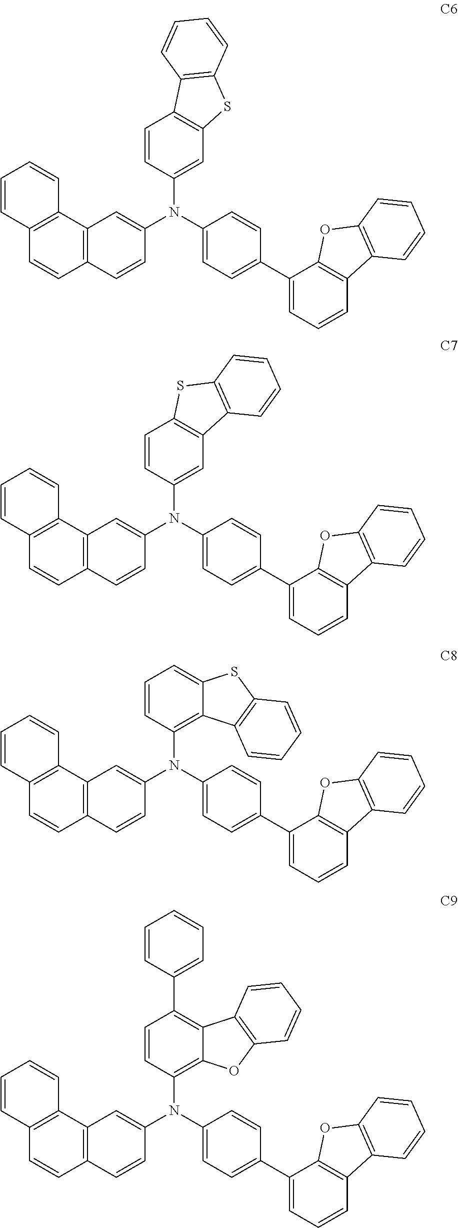 [SMILES: c1ccc2c(c1)ccc1ccc(N(c3ccc(-c4cccc5c4oc4ccccc45)cc3)c3ccc4c(c3)sc3ccccc34)cc12 ; c1ccc2c(c1)ccc1ccc(N(c3ccc(-c4cccc5c4oc4ccccc45)cc3)c3ccc4sc5ccccc5c4c3)cc12 ; c1ccc2c(c1)ccc1ccc(N(c3ccc(-c4cccc5c4oc4ccccc45)cc3)c3cccc4sc5ccccc5c34)cc12 ; c1ccc(-c2ccc(N(c3ccc(-c4cccc5c4oc4ccccc45)cc3)c3ccc4ccc5ccccc5c4c3)c3oc4ccccc4c23)cc1]

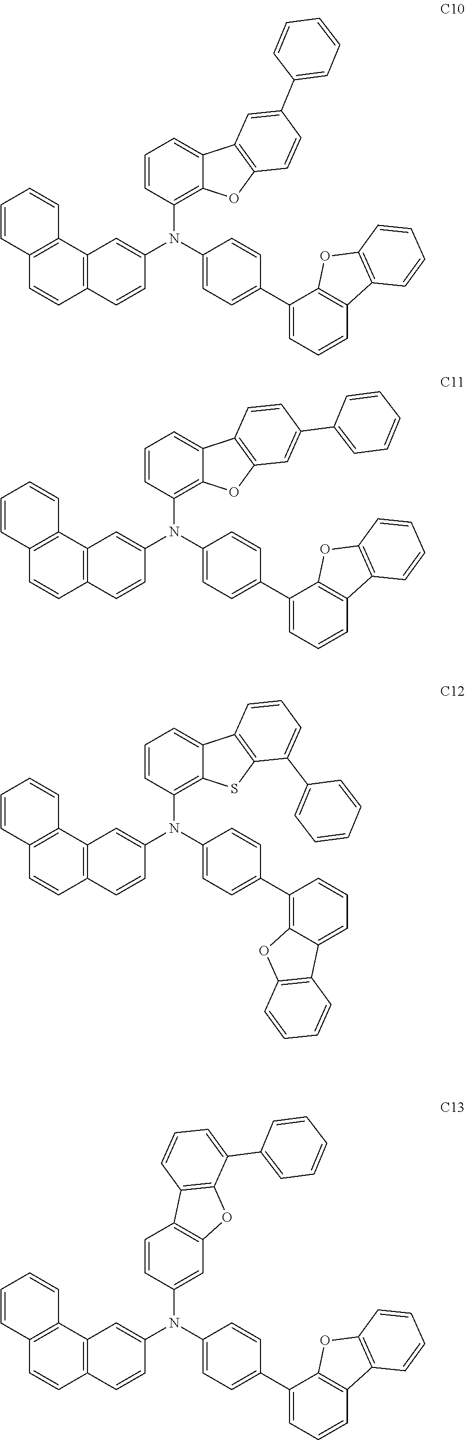 [SMILES: c1ccc(-c2ccc3oc4c(N(c5ccc(-c6cccc7c6oc6ccccc67)cc5)c5ccc6ccc7ccccc7c6c5)cccc4c3c2)cc1 ; c1ccc(-c2ccc3c(c2)oc2c(N(c4ccc(-c5cccc6c5oc5ccccc56)cc4)c4ccc5ccc6ccccc6c5c4)cccc23)cc1 ; c1ccc(-c2cccc3c2sc2c(N(c4ccc(-c5cccc6c5oc5ccccc56)cc4)c4ccc5ccc6ccccc6c5c4)cccc23)cc1 ; c1ccc(-c2cccc3c2oc2cc(N(c4ccc(-c5cccc6c5oc5ccccc56)cc4)c4ccc5ccc6ccccc6c5c4)ccc23)cc1]

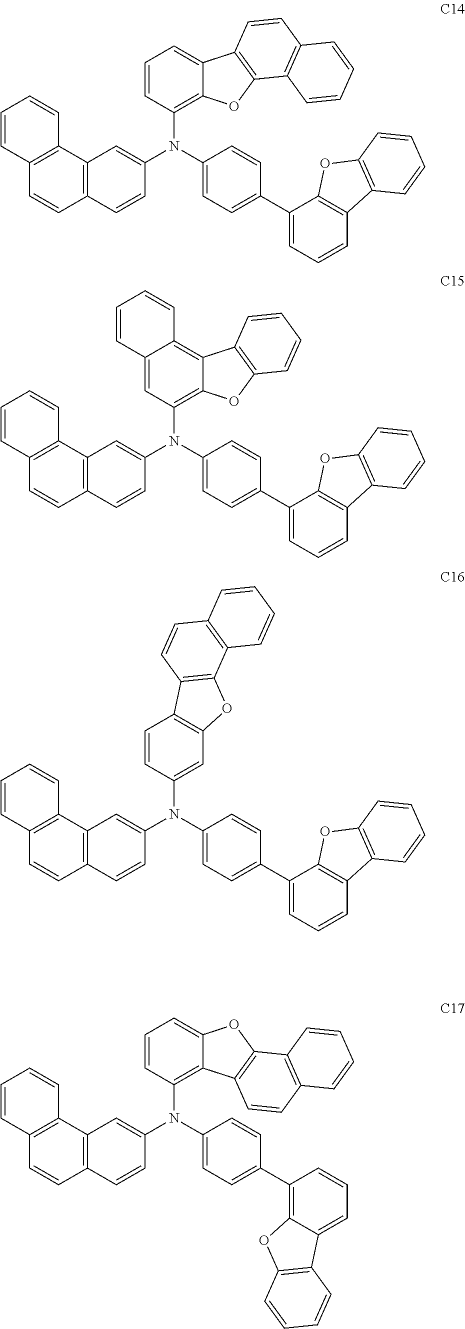 [SMILES: c1ccc2c(c1)ccc1ccc(N(c3ccc(-c4cccc5c4oc4ccccc45)cc3)c3cccc4c3oc3c5ccccc5ccc43)cc12 ; c1ccc2c(c1)ccc1ccc(N(c3ccc(-c4cccc5c4oc4ccccc45)cc3)c3cc4ccccc4c4c3oc3ccccc34)cc12 ; c1ccc2c(c1)ccc1ccc(N(c3ccc(-c4cccc5c4oc4ccccc45)cc3)c3ccc4c(c3)oc3c5ccccc5ccc43)cc12 ; c1ccc2c(c1)ccc1ccc(N(c3ccc(-c4cccc5c4oc4ccccc45)cc3)c3cccc4oc5c6ccccc6ccc5c34)cc12]

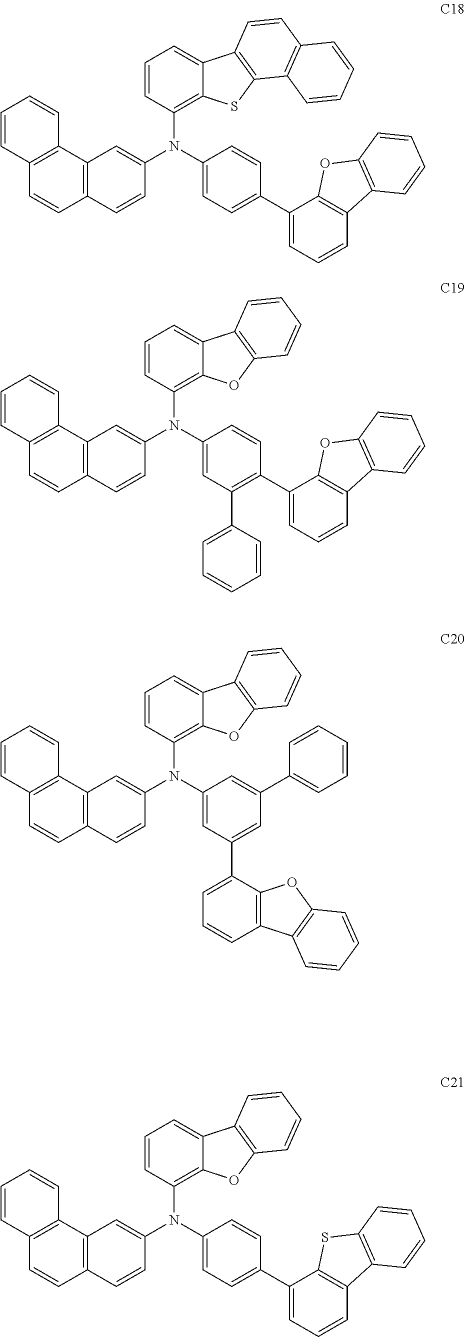 [SMILES: c1ccc2c(c1)ccc1ccc(N(c3ccc(-c4cccc5c4oc4ccccc45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2cccc3c2oc2ccccc23)cc1 ; c1ccc(-c2cc(-c3cccc4c3oc3ccccc34)cc(N(c3ccc4ccc5ccccc5c4c3)c3cccc4c3oc3ccccc34)c2)cc1 ; c1ccc2c(c1)ccc1ccc(N(c3ccc(-c4cccc5c4sc4ccccc45)cc3)c3cccc4c3oc3ccccc34)cc12]

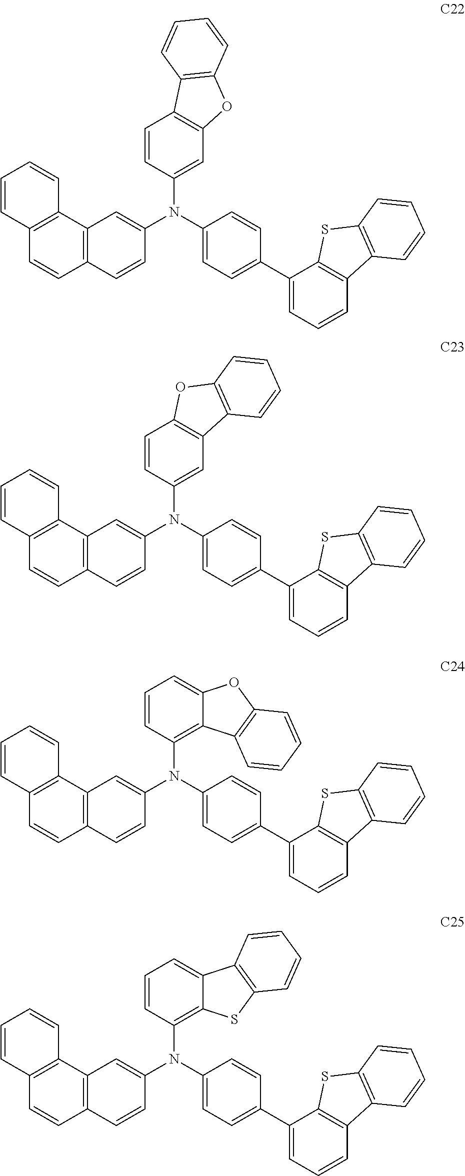 [SMILES: c1ccc2c(c1)ccc1ccc(N(c3ccc(-c4cccc5c4sc4ccccc45)cc3)c3ccc4c(c3)oc3ccccc34)cc12 ; c1ccc2c(c1)ccc1ccc(N(c3ccc(-c4cccc5c4sc4ccccc45)cc3)c3ccc4oc5ccccc5c4c3)cc12 ; c1ccc2c(c1)ccc1ccc(N(c3ccc(-c4cccc5c4sc4ccccc45)cc3)c3cccc4oc5ccccc5c34)cc12 ; c1ccc2c(c1)ccc1ccc(N(c3ccc(-c4cccc5c4sc4ccccc45)cc3)c3cccc4c3sc3ccccc34)cc12]

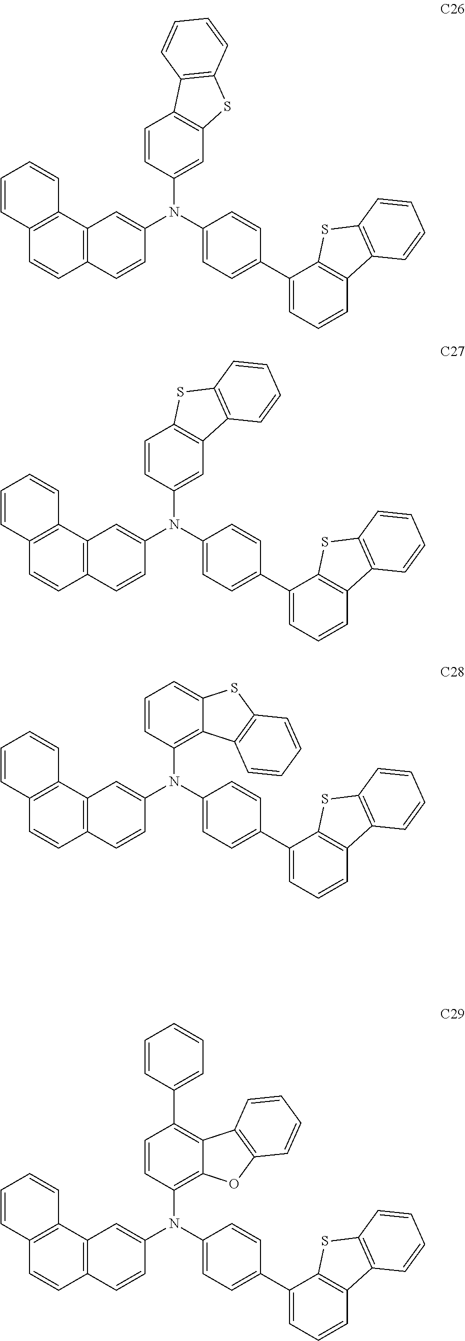 [SMILES: c1ccc2c(c1)ccc1ccc(N(c3ccc(-c4cccc5c4sc4ccccc45)cc3)c3ccc4c(c3)sc3ccccc34)cc12 ; c1ccc2c(c1)ccc1ccc(N(c3ccc(-c4cccc5c4sc4ccccc45)cc3)c3ccc4sc5ccccc5c4c3)cc12 ; c1ccc2c(c1)ccc1ccc(N(c3ccc(-c4cccc5c4sc4ccccc45)cc3)c3cccc4sc5ccccc5c34)cc12 ; c1ccc(-c2ccc(N(c3ccc(-c4cccc5c4sc4ccccc45)cc3)c3ccc4ccc5ccccc5c4c3)c3oc4ccccc4c23)cc1]

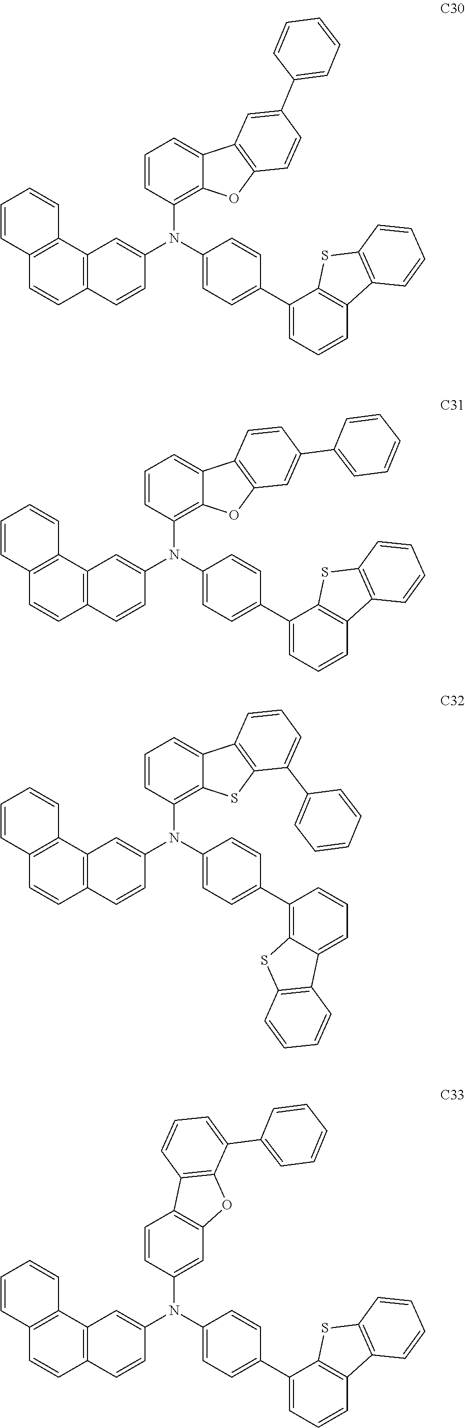 [SMILES: c1ccc(-c2ccc3oc4c(N(c5ccc(-c6cccc7c6sc6ccccc67)cc5)c5ccc6ccc7ccccc7c6c5)cccc4c3c2)cc1 ; c1ccc(-c2ccc3c(c2)oc2c(N(c4ccc(-c5cccc6c5sc5ccccc56)cc4)c4ccc5ccc6ccccc6c5c4)cccc23)cc1 ; c1ccc(-c2cccc3c2sc2c(N(c4ccc(-c5cccc6c5sc5ccccc56)cc4)c4ccc5ccc6ccccc6c5c4)cccc23)cc1 ; c1ccc(-c2cccc3c2oc2cc(N(c4ccc(-c5cccc6c5sc5ccccc56)cc4)c4ccc5ccc6ccccc6c5c4)ccc23)cc1]

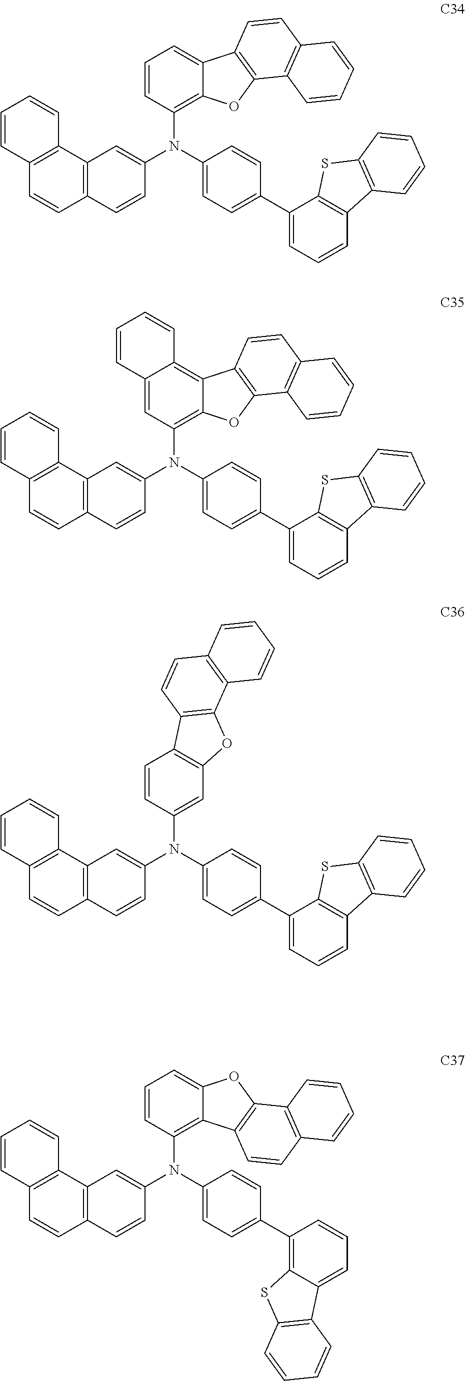 [SMILES: c1ccc2c(c1)ccc1ccc(N(c3ccc(-c4cccc5c4sc4ccccc45)cc3)c3cccc4c3oc3c5ccccc5ccc43)cc12 ; c1ccc2c(c1)ccc1ccc(N(c3ccc(-c4cccc5c4sc4ccccc45)cc3)c3cc4ccccc4c4c3oc3c5ccccc5ccc34)cc12 ; c1ccc2c(c1)ccc1ccc(N(c3ccc(-c4cccc5c4sc4ccccc45)cc3)c3ccc4c(c3)oc3c5ccccc5ccc43)cc12 ; c1ccc2c(c1)ccc1ccc(N(c3ccc(-c4cccc5c4sc4ccccc45)cc3)c3cccc4oc5c6ccccc6ccc5c34)cc12]

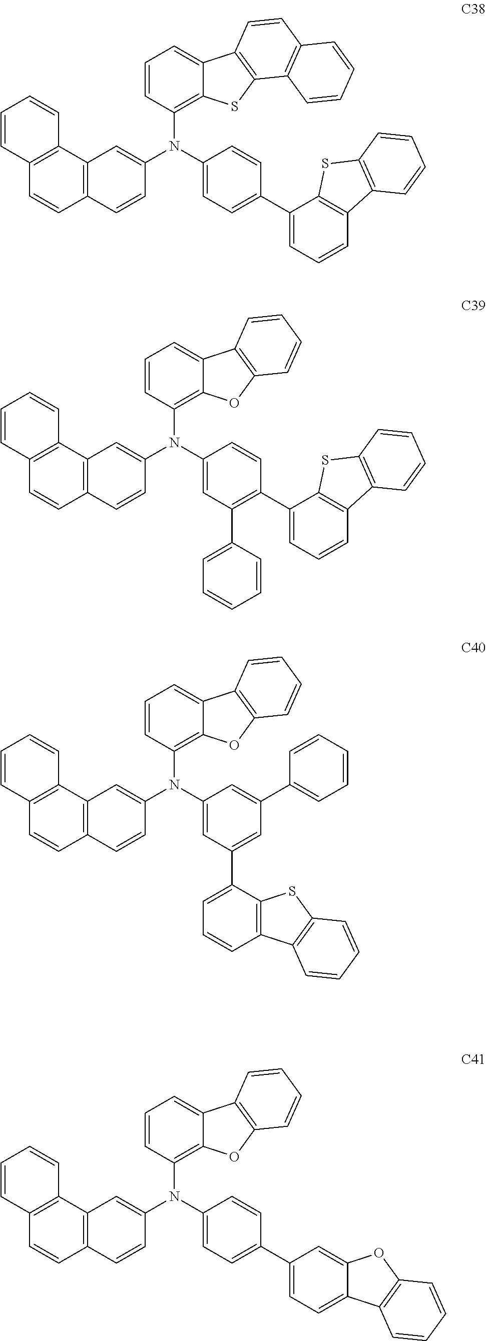 [SMILES: c1ccc2c(c1)ccc1ccc(N(c3ccc(-c4cccc5c4sc4ccccc45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2cccc3c2sc2ccccc23)cc1 ; c1ccc(-c2cc(-c3cccc4c3sc3ccccc34)cc(N(c3ccc4ccc5ccccc5c4c3)c3cccc4c3oc3ccccc34)c2)cc1 ; c1ccc2c(c1)ccc1ccc(N(c3ccc(-c4ccc5c(c4)oc4ccccc45)cc3)c3cccc4c3oc3ccccc34)cc12]

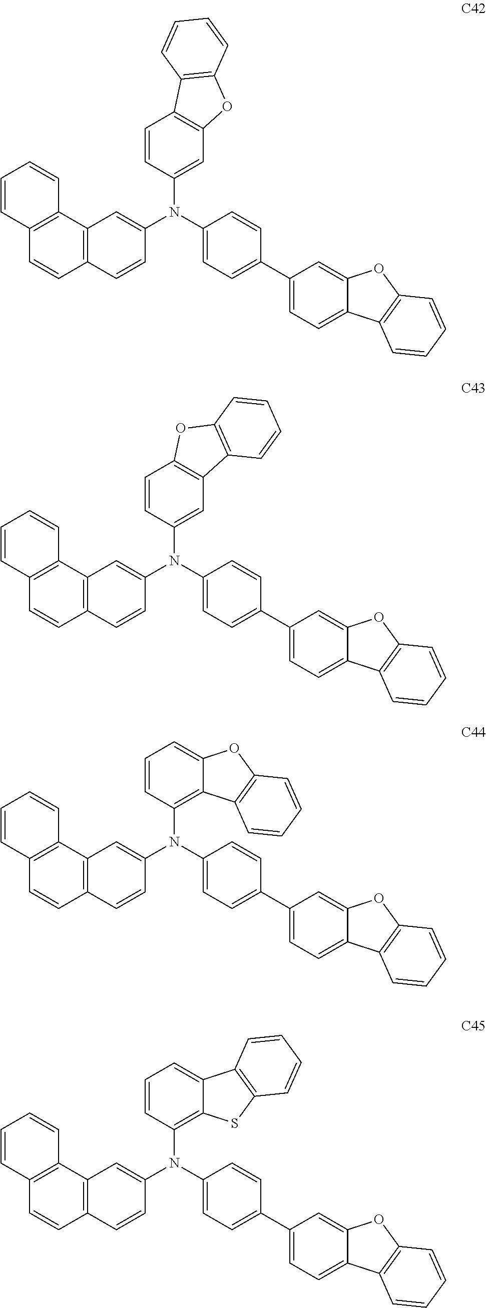 [SMILES: c1ccc2c(c1)ccc1ccc(N(c3ccc(-c4ccc5c(c4)oc4ccccc45)cc3)c3ccc4c(c3)oc3ccccc34)cc12 ; c1ccc2c(c1)ccc1ccc(N(c3ccc(-c4ccc5c(c4)oc4ccccc45)cc3)c3ccc4oc5ccccc5c4c3)cc12 ; c1ccc2c(c1)ccc1ccc(N(c3ccc(-c4ccc5c(c4)oc4ccccc45)cc3)c3cccc4oc5ccccc5c34)cc12 ; c1ccc2c(c1)ccc1ccc(N(c3ccc(-c4ccc5c(c4)oc4ccccc45)cc3)c3cccc4c3sc3ccccc34)cc12]

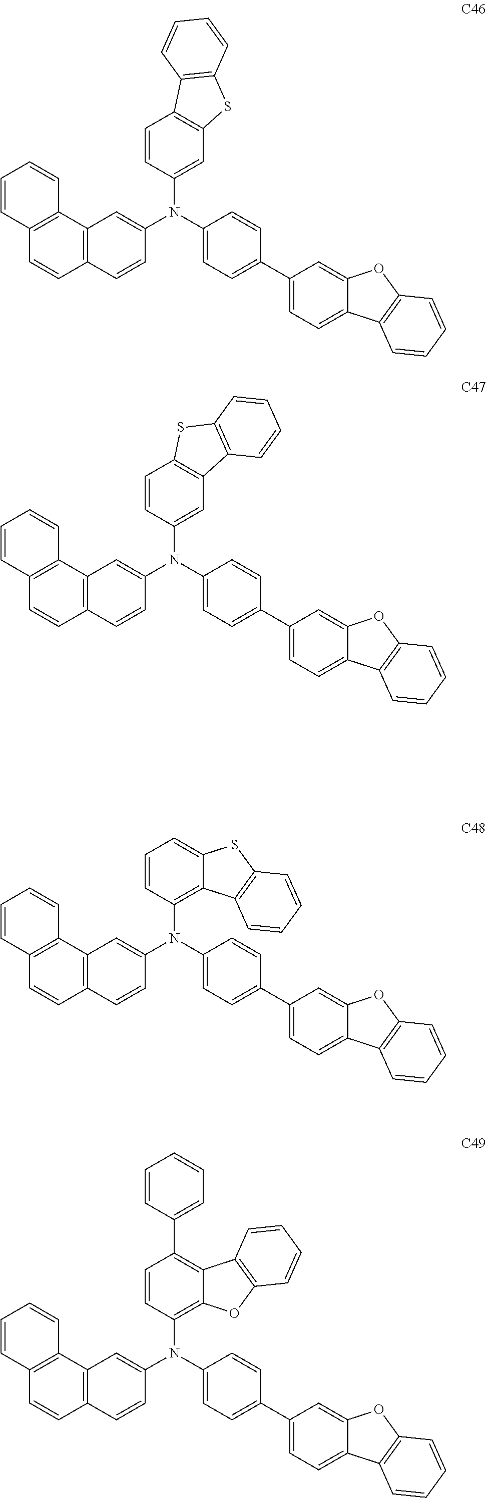 [SMILES: c1ccc2c(c1)ccc1ccc(N(c3ccc(-c4ccc5c(c4)oc4ccccc45)cc3)c3ccc4c(c3)sc3ccccc34)cc12 ; c1ccc2c(c1)ccc1ccc(N(c3ccc(-c4ccc5c(c4)oc4ccccc45)cc3)c3ccc4sc5ccccc5c4c3)cc12 ; c1ccc2c(c1)ccc1ccc(N(c3ccc(-c4ccc5c(c4)oc4ccccc45)cc3)c3cccc4sc5ccccc5c34)cc12 ; c1ccc(-c2ccc(N(c3ccc(-c4ccc5c(c4)oc4ccccc45)cc3)c3ccc4ccc5ccccc5c4c3)c3oc4ccccc4c23)cc1]

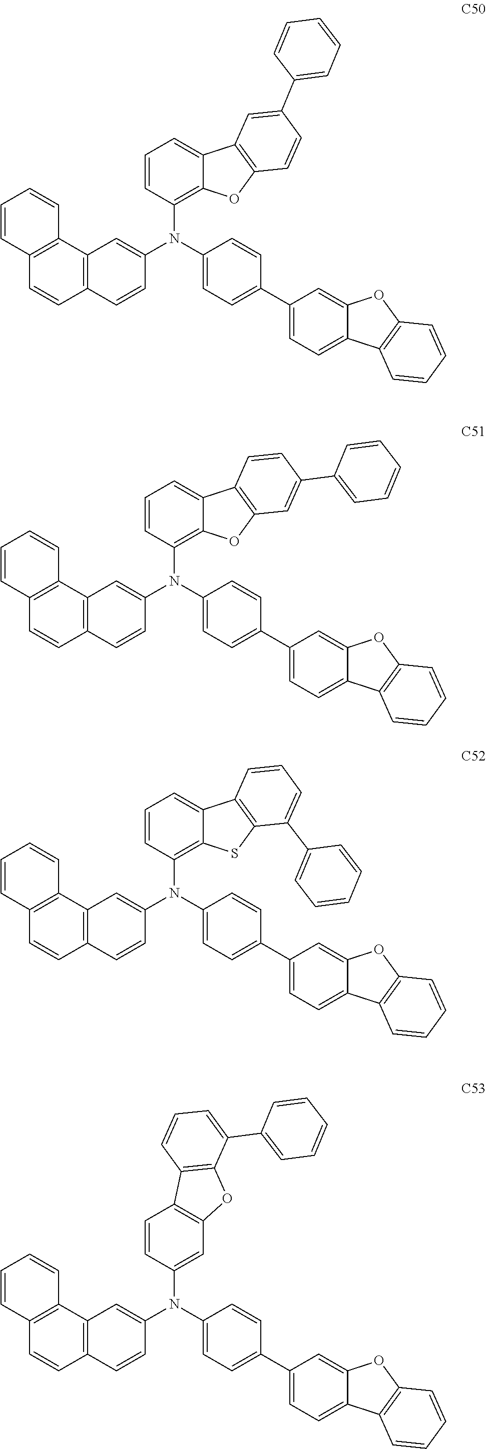 [SMILES: c1ccc(-c2ccc3oc4c(N(c5ccc(-c6ccc7c(c6)oc6ccccc67)cc5)c5ccc6ccc7ccccc7c6c5)cccc4c3c2)cc1 ; c1ccc(-c2ccc3c(c2)oc2c(N(c4ccc(-c5ccc6c(c5)oc5ccccc56)cc4)c4ccc5ccc6ccccc6c5c4)cccc23)cc1 ; c1ccc(-c2cccc3c2sc2c(N(c4ccc(-c5ccc6c(c5)oc5ccccc56)cc4)c4ccc5ccc6ccccc6c5c4)cccc23)cc1 ; c1ccc(-c2cccc3c2oc2cc(N(c4ccc(-c5ccc6c(c5)oc5ccccc56)cc4)c4ccc5ccc6ccccc6c5c4)ccc23)cc1]

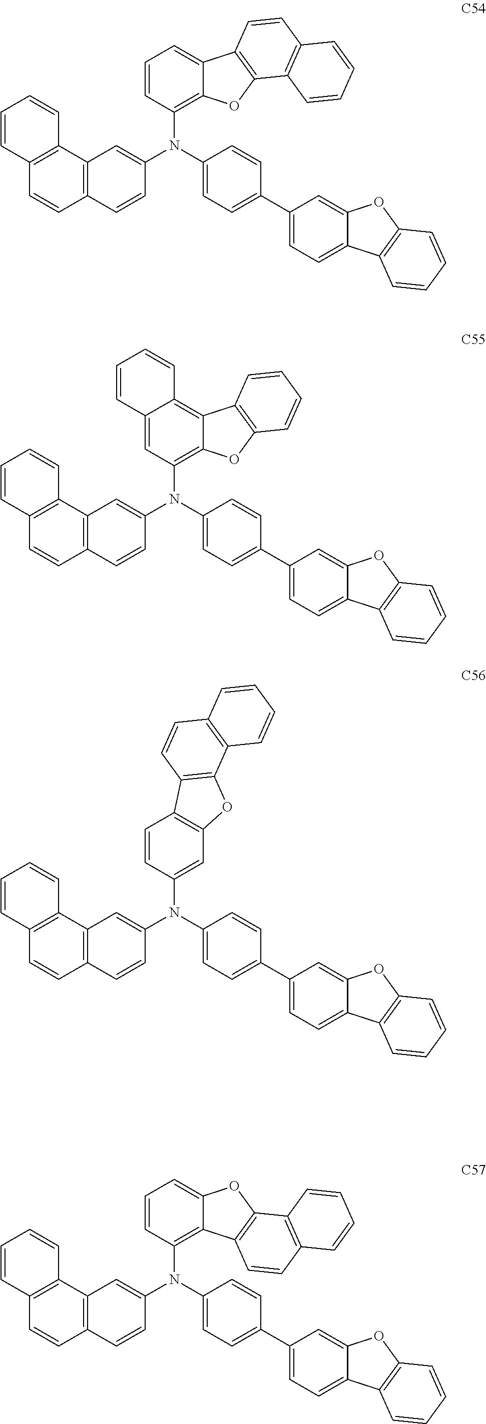 [SMILES: c1ccc2c(c1)ccc1ccc(N(c3ccc(-c4ccc5c(c4)oc4ccccc45)cc3)c3cccc4c3oc3c5ccccc5ccc43)cc12 ; c1ccc2c(c1)ccc1ccc(N(c3ccc(-c4ccc5c(c4)oc4ccccc45)cc3)c3cc4ccccc4c4c3oc3ccccc34)cc12 ; c1ccc2c(c1)ccc1ccc(N(c3ccc(-c4ccc5c(c4)oc4ccccc45)cc3)c3ccc4c(c3)oc3c5ccccc5ccc43)cc12 ; c1ccc2c(c1)ccc1ccc(N(c3ccc(-c4ccc5c(c4)oc4ccccc45)cc3)c3cccc4oc5c6ccccc6ccc5c34)cc12]

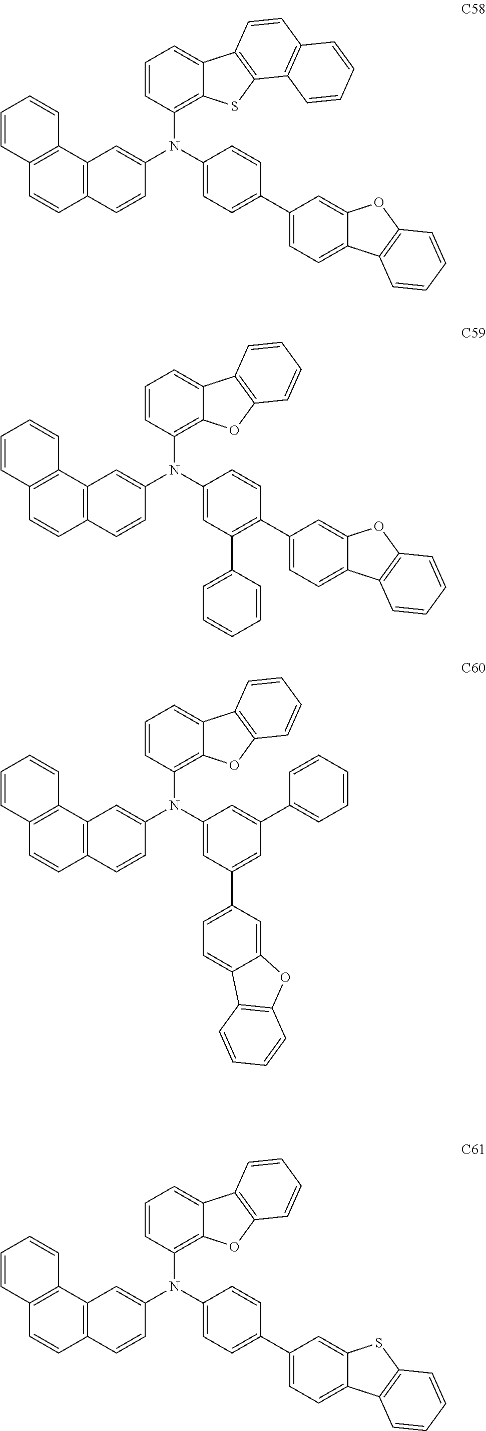 [SMILES: c1ccc2c(c1)ccc1ccc(N(c3ccc(-c4ccc5c(c4)oc4ccccc45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2ccc3c(c2)oc2ccccc23)cc1 ; c1ccc(-c2cc(-c3ccc4c(c3)oc3ccccc34)cc(N(c3ccc4ccc5ccccc5c4c3)c3cccc4c3oc3ccccc34)c2)cc1 ; c1ccc2c(c1)ccc1ccc(N(c3ccc(-c4ccc5c(c4)sc4ccccc45)cc3)c3cccc4c3oc3ccccc34)cc12]

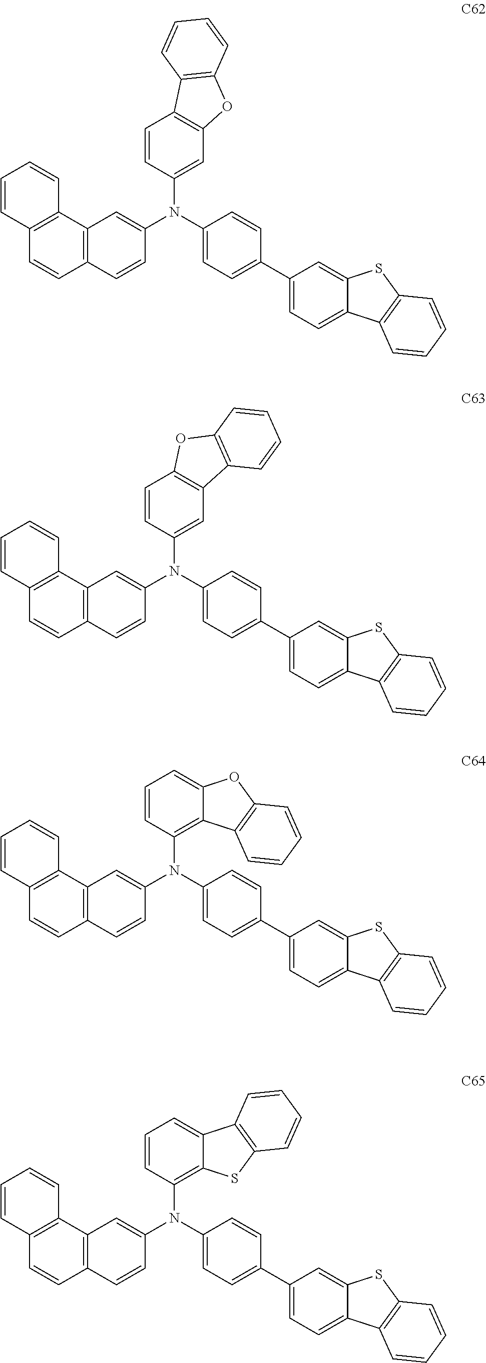 [SMILES: c1ccc2c(c1)ccc1ccc(N(c3ccc(-c4ccc5c(c4)sc4ccccc45)cc3)c3ccc4c(c3)oc3ccccc34)cc12 ; c1ccc2c(c1)ccc1ccc(N(c3ccc(-c4ccc5c(c4)sc4ccccc45)cc3)c3ccc4oc5ccccc5c4c3)cc12 ; c1ccc2c(c1)ccc1ccc(N(c3ccc(-c4ccc5c(c4)sc4ccccc45)cc3)c3cccc4oc5ccccc5c34)cc12 ; c1ccc2c(c1)ccc1ccc(N(c3ccc(-c4ccc5c(c4)sc4ccccc45)cc3)c3cccc4c3sc3ccccc34)cc12]

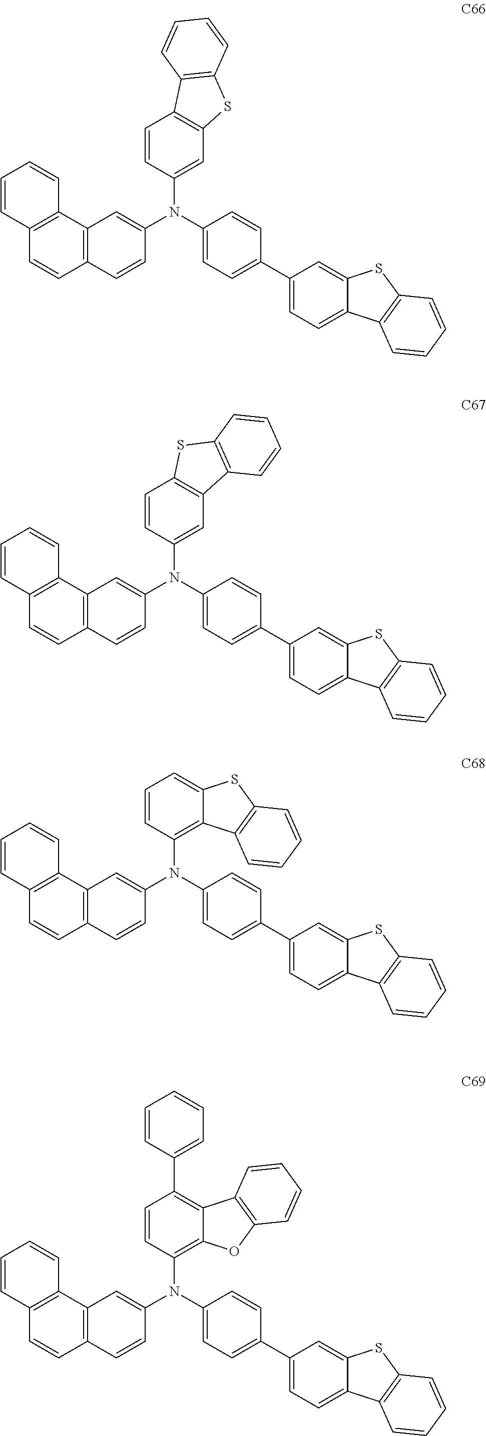 [SMILES: c1ccc2c(c1)ccc1ccc(N(c3ccc(-c4ccc5c(c4)sc4ccccc45)cc3)c3ccc4c(c3)sc3ccccc34)cc12 ; c1ccc2c(c1)ccc1ccc(N(c3ccc(-c4ccc5c(c4)sc4ccccc45)cc3)c3ccc4sc5ccccc5c4c3)cc12 ; c1ccc2c(c1)ccc1ccc(N(c3ccc(-c4ccc5c(c4)sc4ccccc45)cc3)c3cccc4sc5ccccc5c34)cc12 ; c1ccc(-c2ccc(N(c3ccc(-c4ccc5c(c4)sc4ccccc45)cc3)c3ccc4ccc5ccccc5c4c3)c3oc4ccccc4c23)cc1]

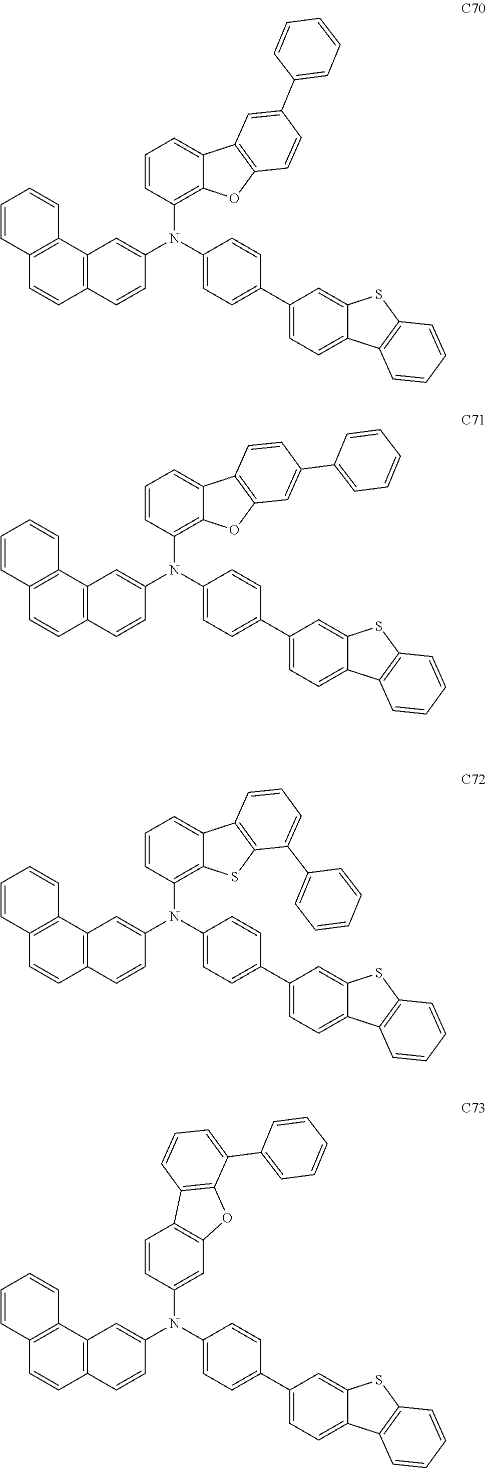 [SMILES: c1ccc(-c2ccc3oc4c(N(c5ccc(-c6ccc7c(c6)sc6ccccc67)cc5)c5ccc6ccc7ccccc7c6c5)cccc4c3c2)cc1 ; c1ccc(-c2ccc3c(c2)oc2c(N(c4ccc(-c5ccc6c(c5)sc5ccccc56)cc4)c4ccc5ccc6ccccc6c5c4)cccc23)cc1 ; c1ccc(-c2cccc3c2sc2c(N(c4ccc(-c5ccc6c(c5)sc5ccccc56)cc4)c4ccc5ccc6ccccc6c5c4)cccc23)cc1 ; c1ccc(-c2cccc3c2oc2cc(N(c4ccc(-c5ccc6c(c5)sc5ccccc56)cc4)c4ccc5ccc6ccccc6c5c4)ccc23)cc1]

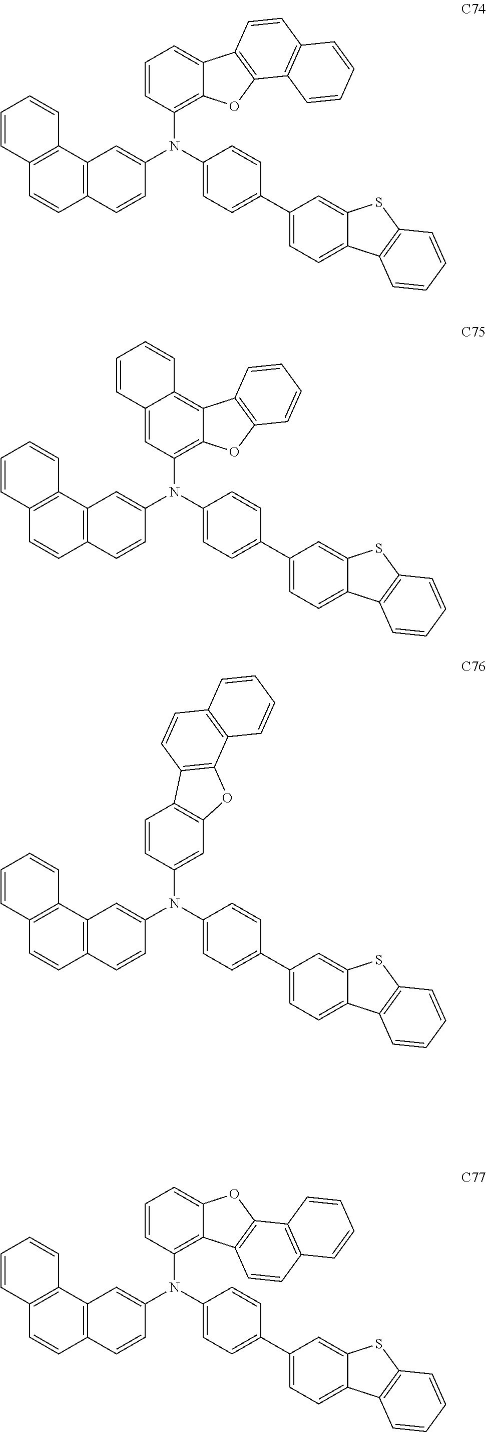 [SMILES: c1ccc2c(c1)ccc1ccc(N(c3ccc(-c4ccc5c(c4)sc4ccccc45)cc3)c3cccc4c3oc3c5ccccc5ccc43)cc12 ; c1ccc2c(c1)ccc1ccc(N(c3ccc(-c4ccc5c(c4)sc4ccccc45)cc3)c3cc4ccccc4c4c3oc3ccccc34)cc12 ; c1ccc2c(c1)ccc1ccc(N(c3ccc(-c4ccc5c(c4)sc4ccccc45)cc3)c3ccc4c(c3)oc3c5ccccc5ccc43)cc12 ; c1ccc2c(c1)ccc1ccc(N(c3ccc(-c4ccc5c(c4)sc4ccccc45)cc3)c3cccc4oc5c6ccccc6ccc5c34)cc12]

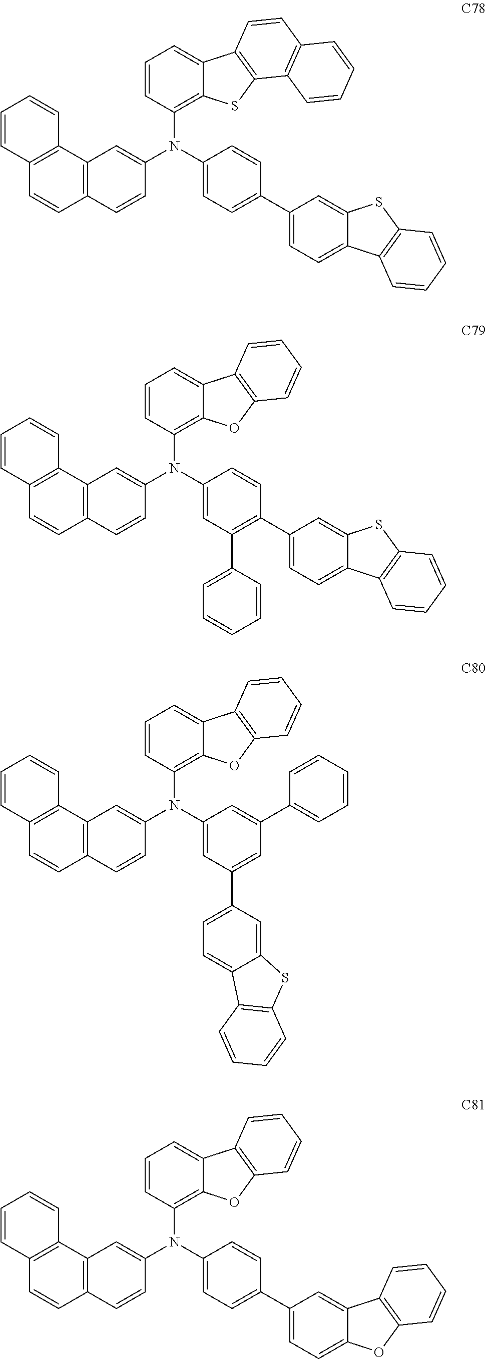 [SMILES: c1ccc2c(c1)ccc1ccc(N(c3ccc(-c4ccc5c(c4)sc4ccccc45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2ccc3c(c2)sc2ccccc23)cc1 ; c1ccc(-c2cc(-c3ccc4c(c3)sc3ccccc34)cc(N(c3ccc4ccc5ccccc5c4c3)c3cccc4c3oc3ccccc34)c2)cc1 ; c1ccc2c(c1)ccc1ccc(N(c3ccc(-c4ccc5oc6ccccc6c5c4)cc3)c3cccc4c3oc3ccccc34)cc12]

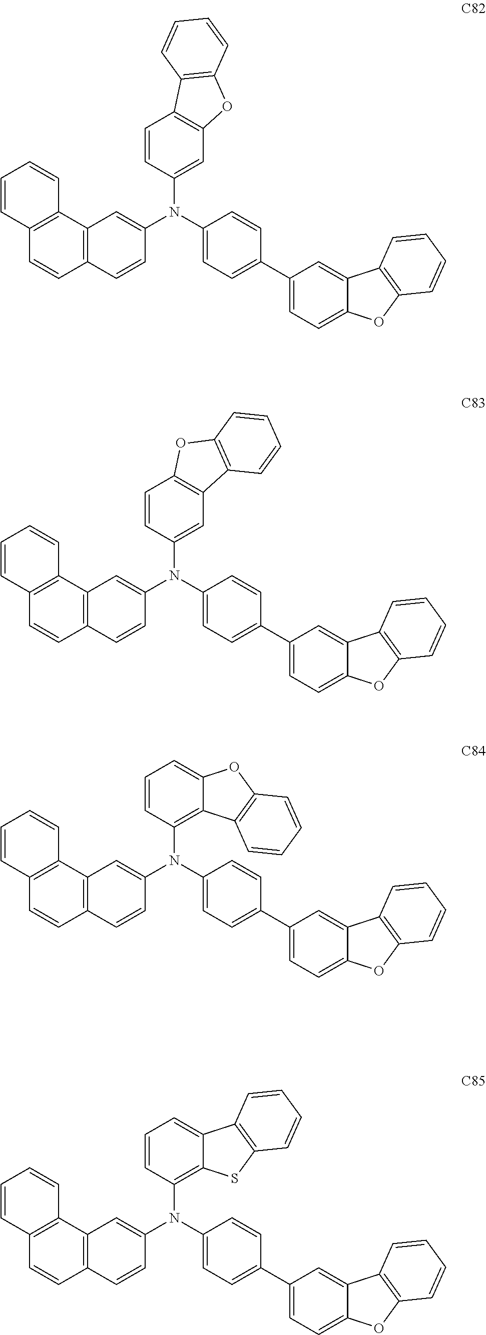 [SMILES: c1ccc2c(c1)ccc1ccc(N(c3ccc(-c4ccc5oc6ccccc6c5c4)cc3)c3ccc4c(c3)oc3ccccc34)cc12 ; c1ccc2c(c1)ccc1ccc(N(c3ccc(-c4ccc5oc6ccccc6c5c4)cc3)c3ccc4oc5ccccc5c4c3)cc12 ; c1ccc2c(c1)ccc1ccc(N(c3ccc(-c4ccc5oc6ccccc6c5c4)cc3)c3cccc4oc5ccccc5c34)cc12 ; c1ccc2c(c1)ccc1ccc(N(c3ccc(-c4ccc5oc6ccccc6c5c4)cc3)c3cccc4c3sc3ccccc34)cc12]

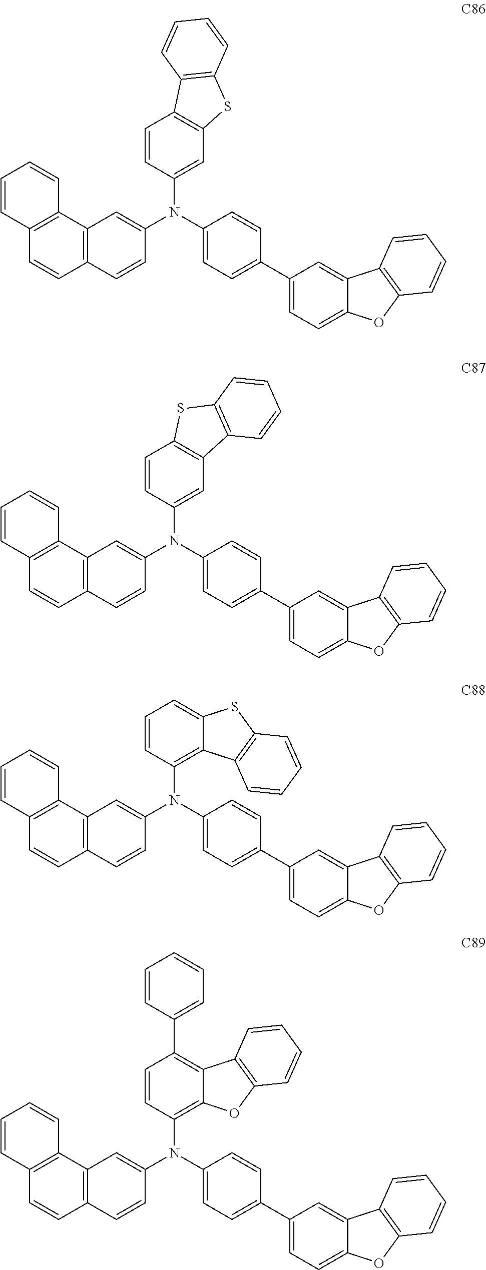 [SMILES: c1ccc2c(c1)ccc1ccc(N(c3ccc(-c4ccc5oc6ccccc6c5c4)cc3)c3ccc4c(c3)sc3ccccc34)cc12 ; c1ccc2c(c1)ccc1ccc(N(c3ccc(-c4ccc5oc6ccccc6c5c4)cc3)c3ccc4sc5ccccc5c4c3)cc12 ; c1ccc2c(c1)ccc1ccc(N(c3ccc(-c4ccc5oc6ccccc6c5c4)cc3)c3cccc4sc5ccccc5c34)cc12 ; c1ccc(-c2ccc(N(c3ccc(-c4ccc5oc6ccccc6c5c4)cc3)c3ccc4ccc5ccccc5c4c3)c3oc4ccccc4c23)cc1]

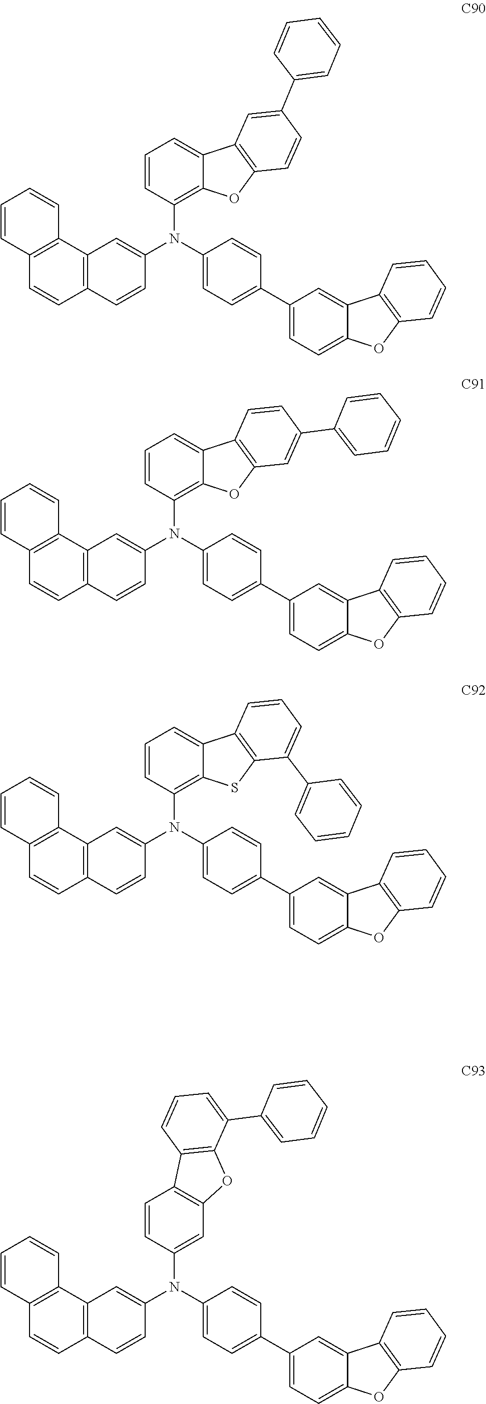

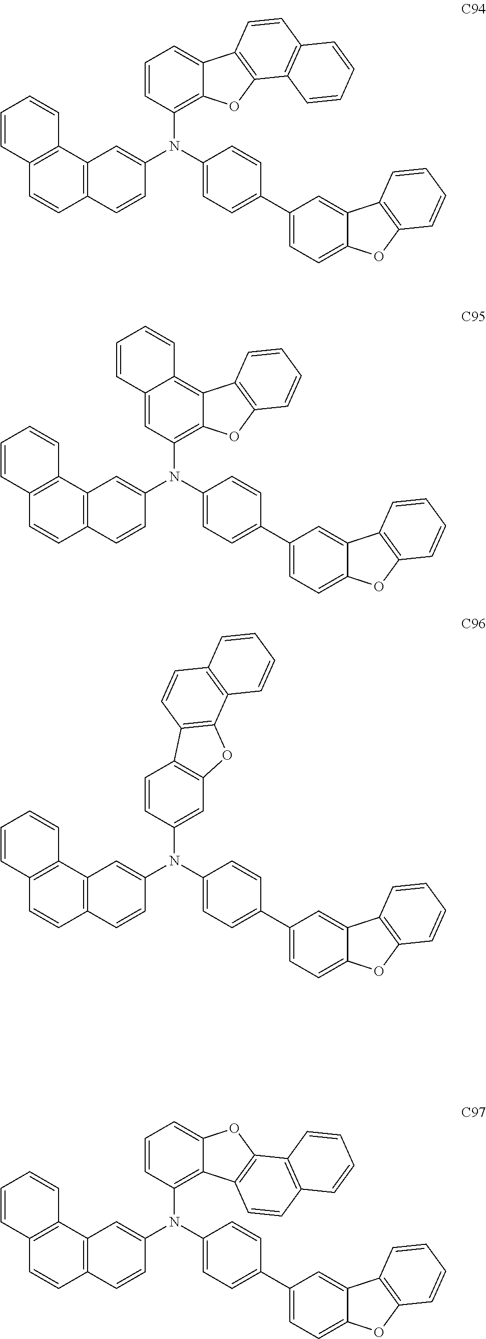 [SMILES: c1ccc2c(c1)ccc1ccc(N(c3ccc(-c4ccc5oc6ccccc6c5c4)cc3)c3cccc4c3oc3c5ccccc5ccc43)cc12 ; c1ccc2c(c1)ccc1ccc(N(c3ccc(-c4ccc5oc6ccccc6c5c4)cc3)c3cc4ccccc4c4c3oc3ccccc34)cc12 ; c1ccc2c(c1)ccc1ccc(N(c3ccc(-c4ccc5oc6ccccc6c5c4)cc3)c3ccc4c(c3)oc3c5ccccc5ccc43)cc12 ; c1ccc2c(c1)ccc1ccc(N(c3ccc(-c4ccc5oc6ccccc6c5c4)cc3)c3cccc4oc5c6ccccc6ccc5c34)cc12]

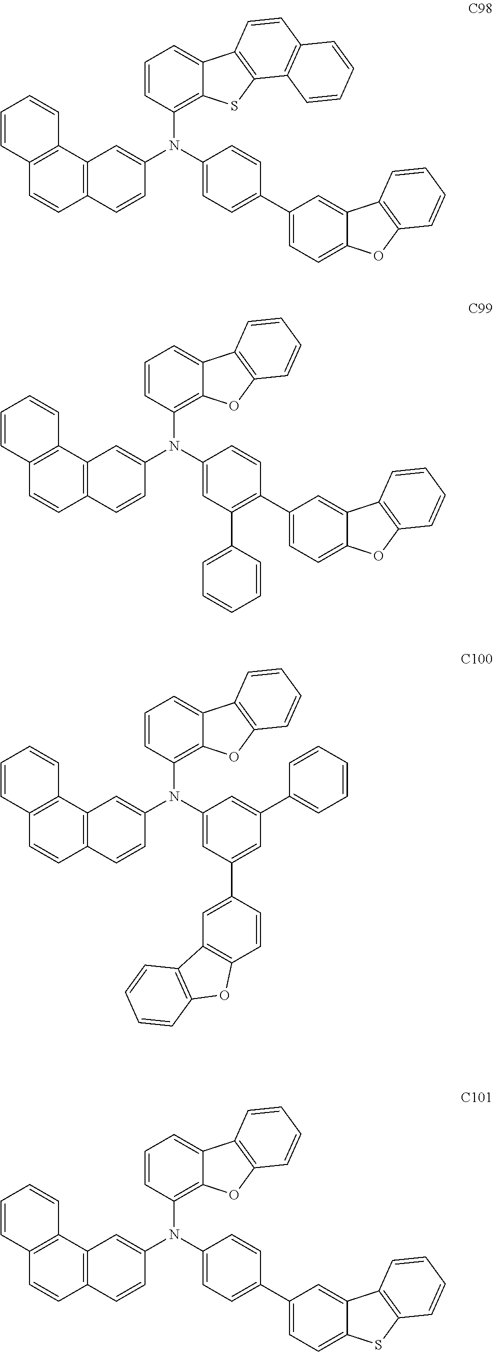 [SMILES: c1ccc2c(c1)ccc1ccc(N(c3ccc(-c4ccc5oc6ccccc6c5c4)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2ccc3oc4ccccc4c3c2)cc1 ; c1ccc(-c2cc(-c3ccc4oc5ccccc5c4c3)cc(N(c3ccc4ccc5ccccc5c4c3)c3cccc4c3oc3ccccc34)c2)cc1 ; c1ccc2c(c1)ccc1ccc(N(c3ccc(-c4ccc5sc6ccccc6c5c4)cc3)c3cccc4c3oc3ccccc34)cc12]

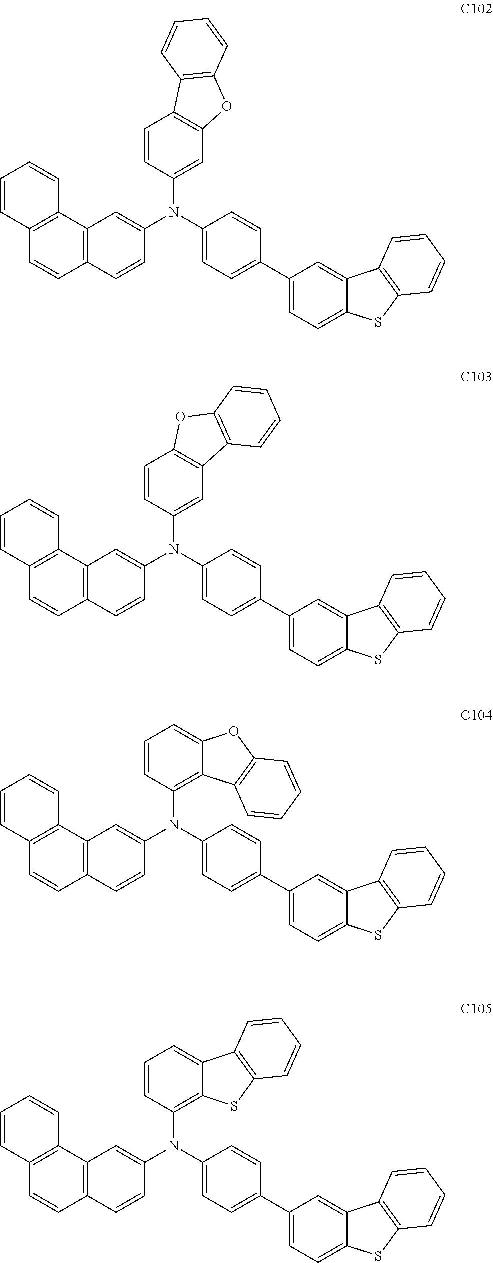 [SMILES: c1ccc2c(c1)ccc1ccc(N(c3ccc(-c4ccc5sc6ccccc6c5c4)cc3)c3ccc4c(c3)oc3ccccc34)cc12 ; c1ccc2c(c1)ccc1ccc(N(c3ccc(-c4ccc5sc6ccccc6c5c4)cc3)c3ccc4oc5ccccc5c4c3)cc12 ; c1ccc2c(c1)ccc1ccc(N(c3ccc(-c4ccc5sc6ccccc6c5c4)cc3)c3cccc4oc5ccccc5c34)cc12 ; c1ccc2c(c1)ccc1ccc(N(c3ccc(-c4ccc5sc6ccccc6c5c4)cc3)c3cccc4c3sc3ccccc34)cc12]

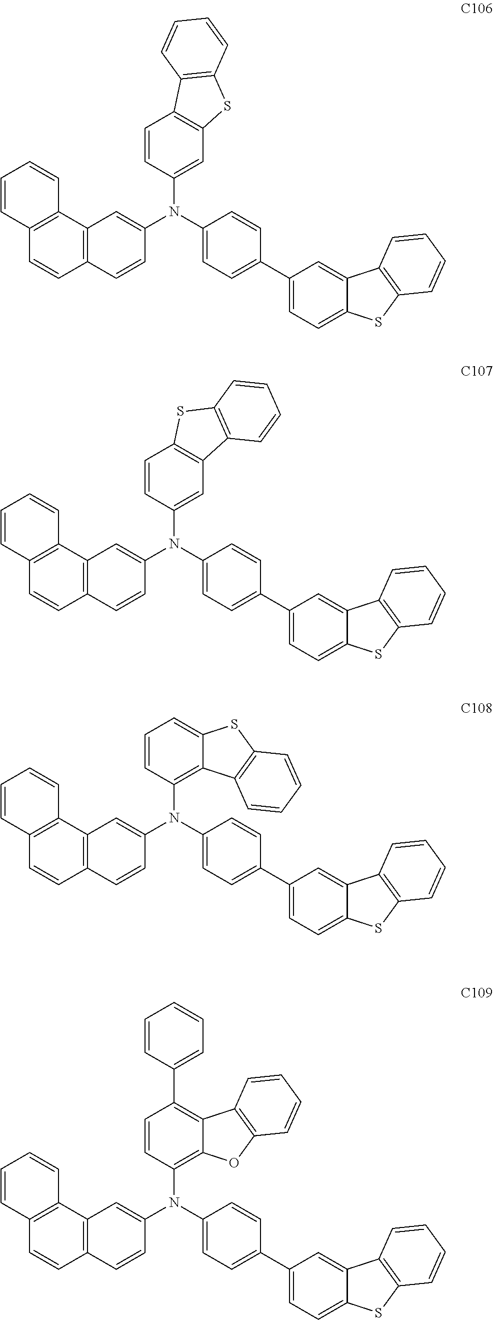 [SMILES: c1ccc2c(c1)ccc1ccc(N(c3ccc(-c4ccc5sc6ccccc6c5c4)cc3)c3ccc4c(c3)sc3ccccc34)cc12 ; c1ccc2c(c1)ccc1ccc(N(c3ccc(-c4ccc5sc6ccccc6c5c4)cc3)c3ccc4sc5ccccc5c4c3)cc12 ; c1ccc2c(c1)ccc1ccc(N(c3ccc(-c4ccc5sc6ccccc6c5c4)cc3)c3cccc4sc5ccccc5c34)cc12 ; c1ccc(-c2ccc(N(c3ccc(-c4ccc5sc6ccccc6c5c4)cc3)c3ccc4ccc5ccccc5c4c3)c3oc4ccccc4c23)cc1]

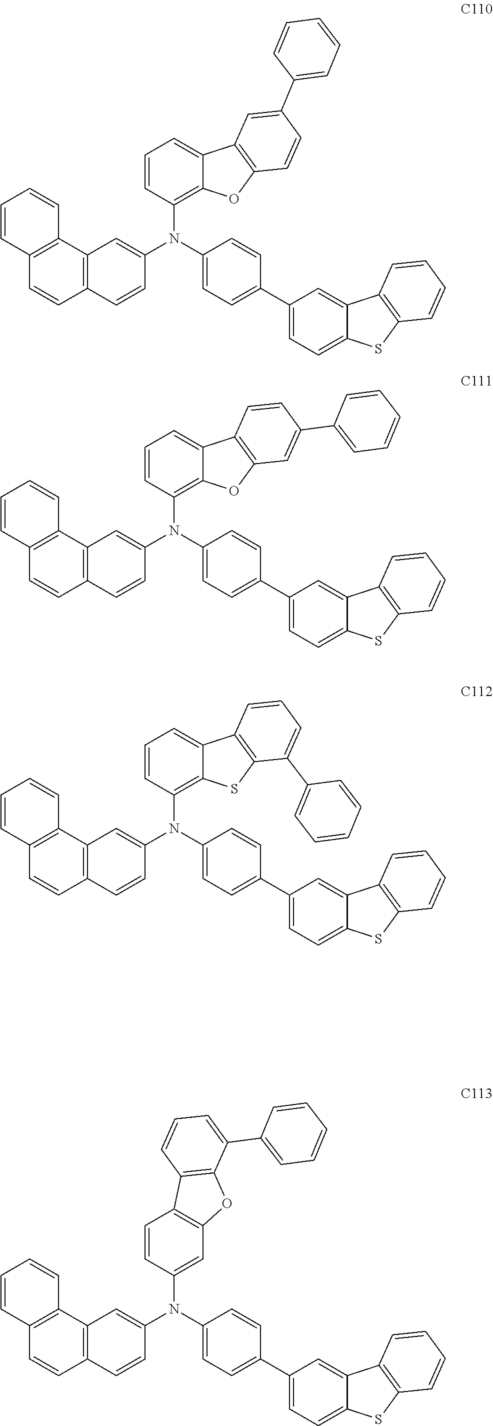

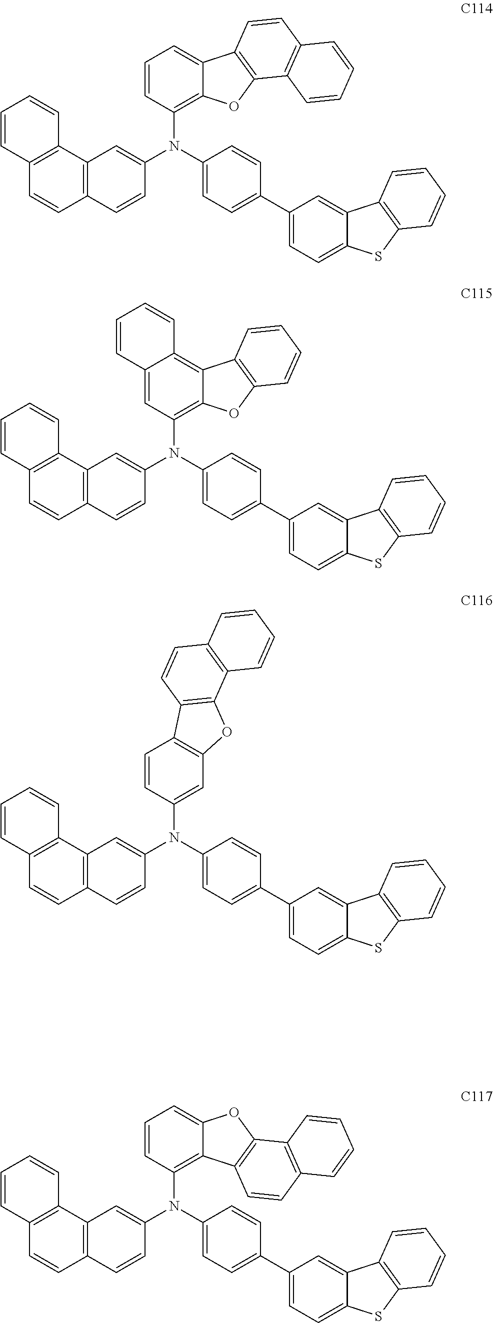 [SMILES: c1ccc2c(c1)ccc1ccc(N(c3ccc(-c4ccc5sc6ccccc6c5c4)cc3)c3cccc4c3oc3c5ccccc5ccc43)cc12 ; c1ccc2c(c1)ccc1ccc(N(c3ccc(-c4ccc5sc6ccccc6c5c4)cc3)c3cc4ccccc4c4c3oc3ccccc34)cc12 ; c1ccc2c(c1)ccc1ccc(N(c3ccc(-c4ccc5sc6ccccc6c5c4)cc3)c3ccc4c(c3)oc3c5ccccc5ccc43)cc12 ; c1ccc2c(c1)ccc1ccc(N(c3ccc(-c4ccc5sc6ccccc6c5c4)cc3)c3cccc4oc5c6ccccc6ccc5c34)cc12]

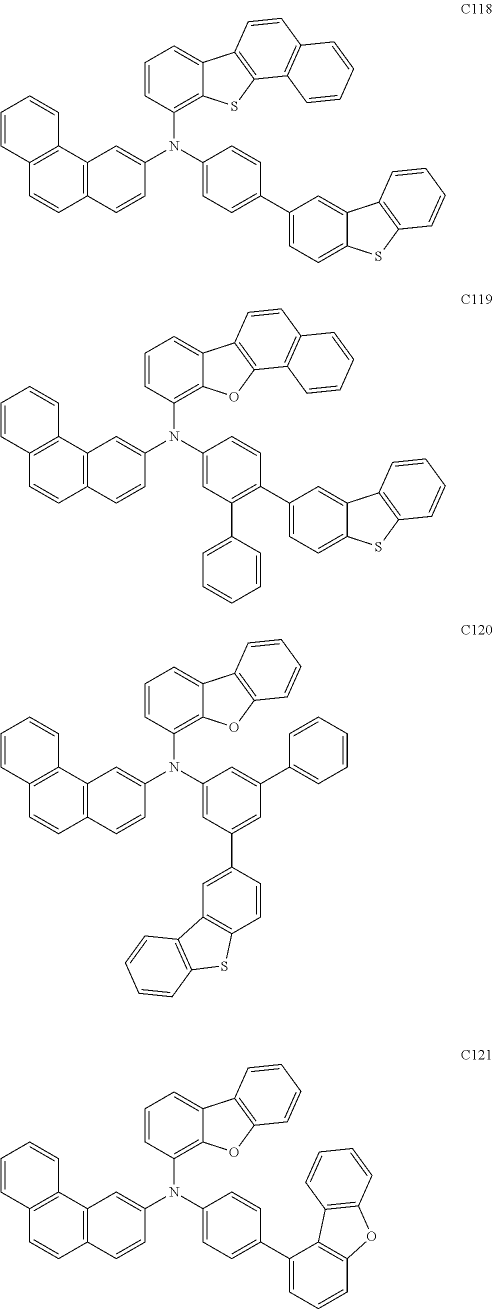

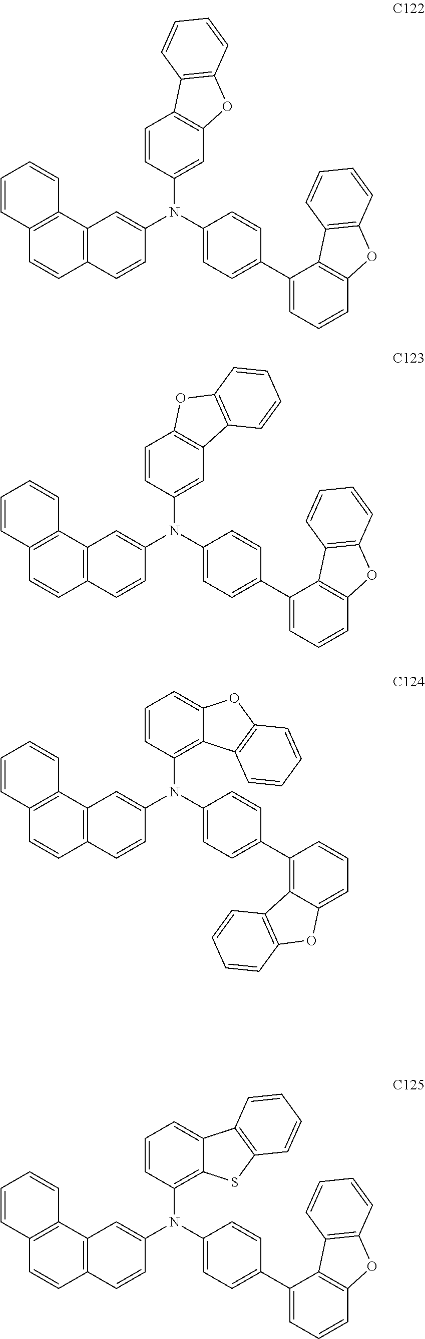 [SMILES: c1ccc2c(c1)ccc1ccc(N(c3ccc(-c4cccc5oc6ccccc6c45)cc3)c3ccc4c(c3)oc3ccccc34)cc12 ; c1ccc2c(c1)ccc1ccc(N(c3ccc(-c4cccc5oc6ccccc6c45)cc3)c3ccc4oc5ccccc5c4c3)cc12 ; c1ccc2c(c1)ccc1ccc(N(c3ccc(-c4cccc5oc6ccccc6c45)cc3)c3cccc4oc5ccccc5c34)cc12 ; c1ccc2c(c1)ccc1ccc(N(c3ccc(-c4cccc5oc6ccccc6c45)cc3)c3cccc4c3sc3ccccc34)cc12]

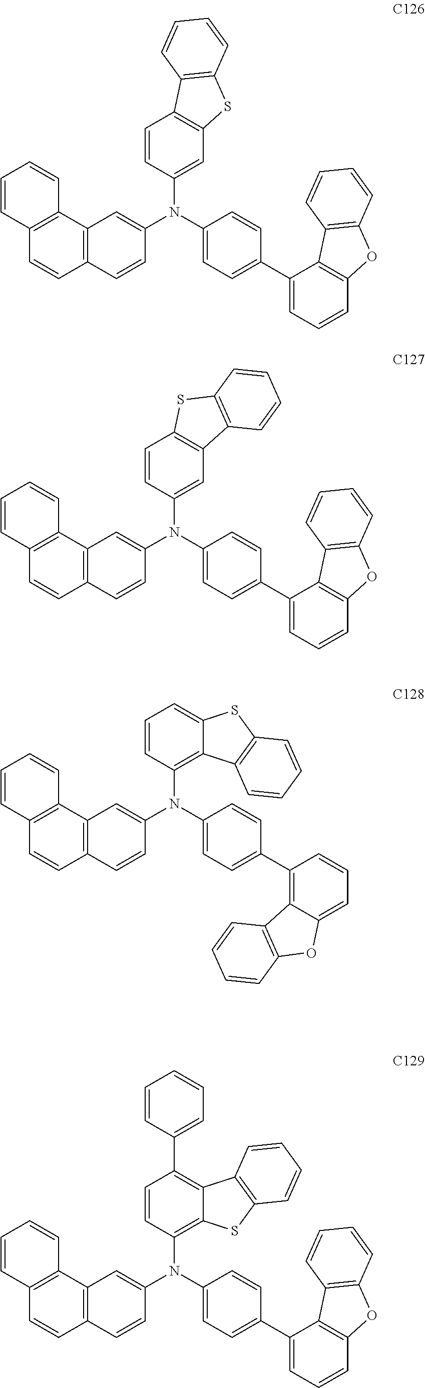 [SMILES: c1ccc2c(c1)ccc1ccc(N(c3ccc(-c4cccc5oc6ccccc6c45)cc3)c3ccc4c(c3)sc3ccccc34)cc12 ; c1ccc2c(c1)ccc1ccc(N(c3ccc(-c4cccc5oc6ccccc6c45)cc3)c3ccc4sc5ccccc5c4c3)cc12 ; c1ccc2c(c1)ccc1ccc(N(c3ccc(-c4cccc5oc6ccccc6c45)cc3)c3cccc4sc5ccccc5c34)cc12 ; c1ccc(-c2ccc(N(c3ccc(-c4cccc5oc6ccccc6c45)cc3)c3ccc4ccc5ccccc5c4c3)c3sc4ccccc4c23)cc1]

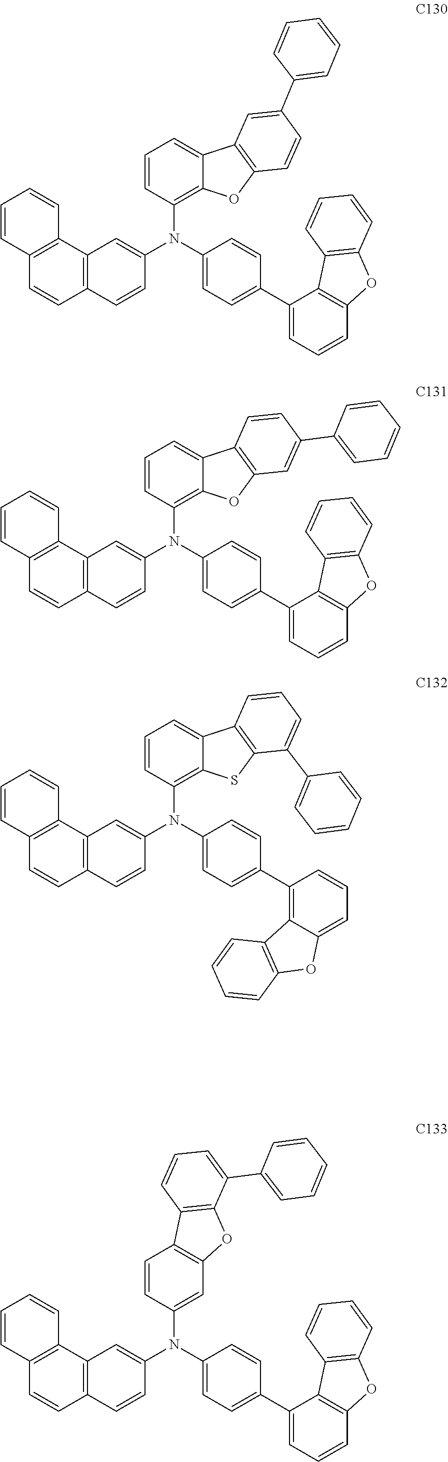 [SMILES: c1ccc(-c2ccc3oc4c(N(c5ccc(-c6cccc7oc8ccccc8c67)cc5)c5ccc6ccc7ccccc7c6c5)cccc4c3c2)cc1 ; c1ccc(-c2ccc3c(c2)oc2c(N(c4ccc(-c5cccc6oc7ccccc7c56)cc4)c4ccc5ccc6ccccc6c5c4)cccc23)cc1 ; c1ccc(-c2cccc3c2sc2c(N(c4ccc(-c5cccc6oc7ccccc7c56)cc4)c4ccc5ccc6ccccc6c5c4)cccc23)cc1 ; c1ccc(-c2cccc3c2oc2cc(N(c4ccc(-c5cccc6oc7ccccc7c56)cc4)c4ccc5ccc6ccccc6c5c4)ccc23)cc1]

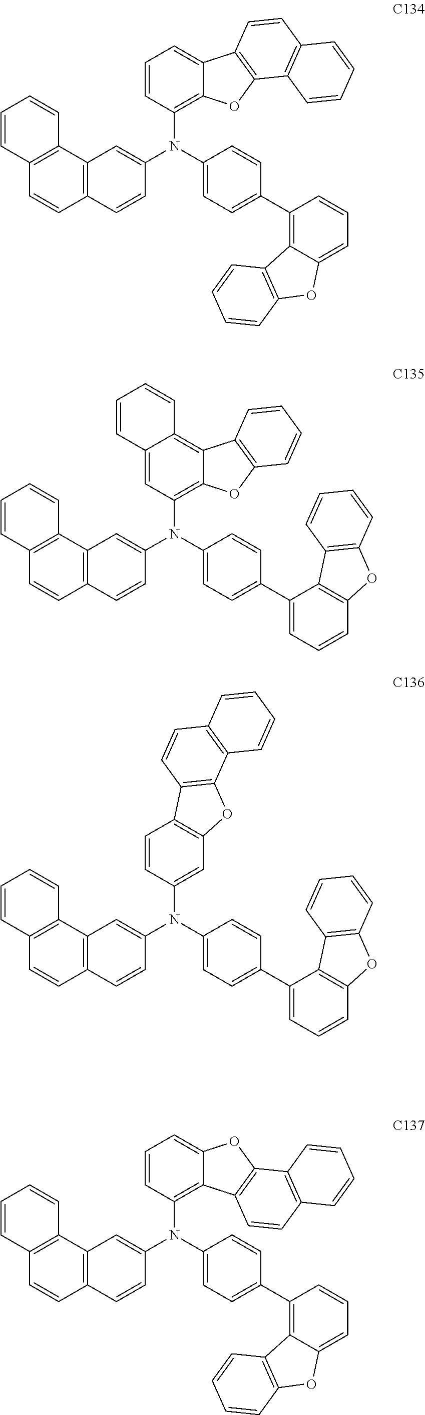 [SMILES: c1ccc2c(c1)ccc1ccc(N(c3ccc(-c4cccc5oc6ccccc6c45)cc3)c3cccc4c3oc3c5ccccc5ccc43)cc12 ; c1ccc2c(c1)ccc1ccc(N(c3ccc(-c4cccc5oc6ccccc6c45)cc3)c3cc4ccccc4c4c3oc3ccccc34)cc12 ; c1ccc2c(c1)ccc1ccc(N(c3ccc(-c4cccc5oc6ccccc6c45)cc3)c3ccc4c(c3)oc3c5ccccc5ccc43)cc12 ; c1ccc2c(c1)ccc1ccc(N(c3ccc(-c4cccc5oc6ccccc6c45)cc3)c3cccc4oc5c6ccccc6ccc5c34)cc12]

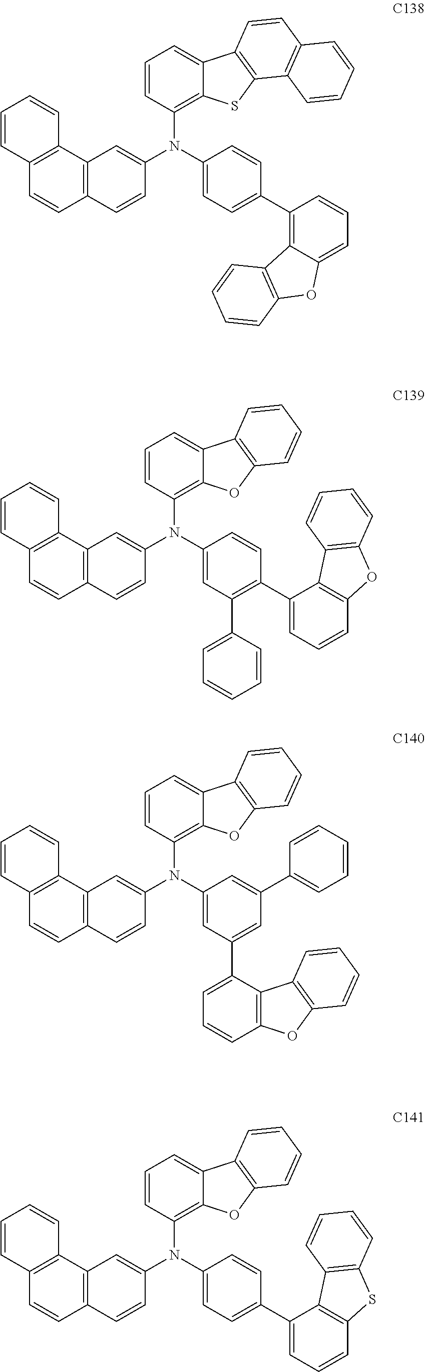 [SMILES: c1ccc2c(c1)ccc1ccc(N(c3ccc(-c4cccc5oc6ccccc6c45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2cccc3oc4ccccc4c23)cc1 ; c1ccc(-c2cc(-c3cccc4oc5ccccc5c34)cc(N(c3ccc4ccc5ccccc5c4c3)c3cccc4c3oc3ccccc34)c2)cc1 ; c1ccc2c(c1)ccc1ccc(N(c3ccc(-c4cccc5sc6ccccc6c45)cc3)c3cccc4c3oc3ccccc34)cc12]

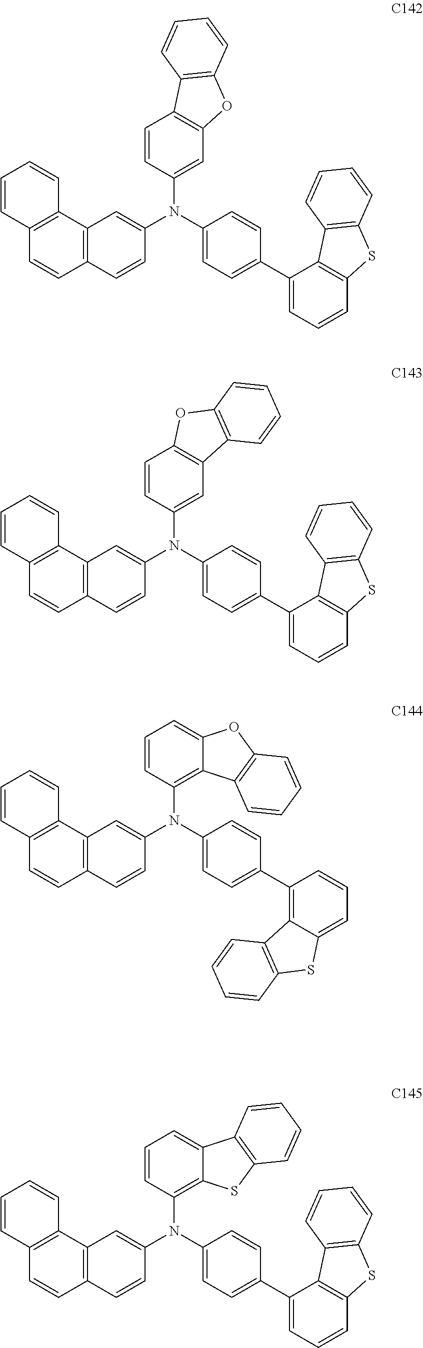 [SMILES: c1ccc2c(c1)ccc1ccc(N(c3ccc(-c4cccc5sc6ccccc6c45)cc3)c3ccc4c(c3)oc3ccccc34)cc12 ; c1ccc2c(c1)ccc1ccc(N(c3ccc(-c4cccc5sc6ccccc6c45)cc3)c3ccc4oc5ccccc5c4c3)cc12 ; c1ccc2c(c1)ccc1ccc(N(c3ccc(-c4cccc5sc6ccccc6c45)cc3)c3cccc4oc5ccccc5c34)cc12 ; c1ccc2c(c1)ccc1ccc(N(c3ccc(-c4cccc5sc6ccccc6c45)cc3)c3cccc4c3sc3ccccc34)cc12]

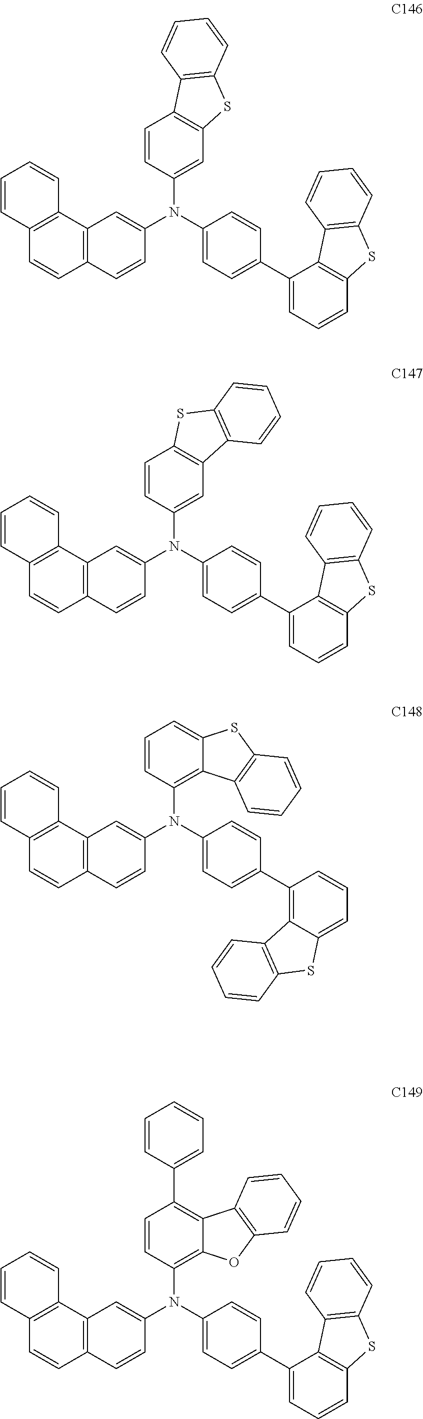 [SMILES: c1ccc2c(c1)ccc1ccc(N(c3ccc(-c4cccc5sc6ccccc6c45)cc3)c3ccc4c(c3)sc3ccccc34)cc12 ; c1ccc2c(c1)ccc1ccc(N(c3ccc(-c4cccc5sc6ccccc6c45)cc3)c3ccc4sc5ccccc5c4c3)cc12 ; c1ccc2c(c1)ccc1ccc(N(c3ccc(-c4cccc5sc6ccccc6c45)cc3)c3cccc4sc5ccccc5c34)cc12 ; c1ccc(-c2ccc(N(c3ccc(-c4cccc5sc6ccccc6c45)cc3)c3ccc4ccc5ccccc5c4c3)c3oc4ccccc4c23)cc1]

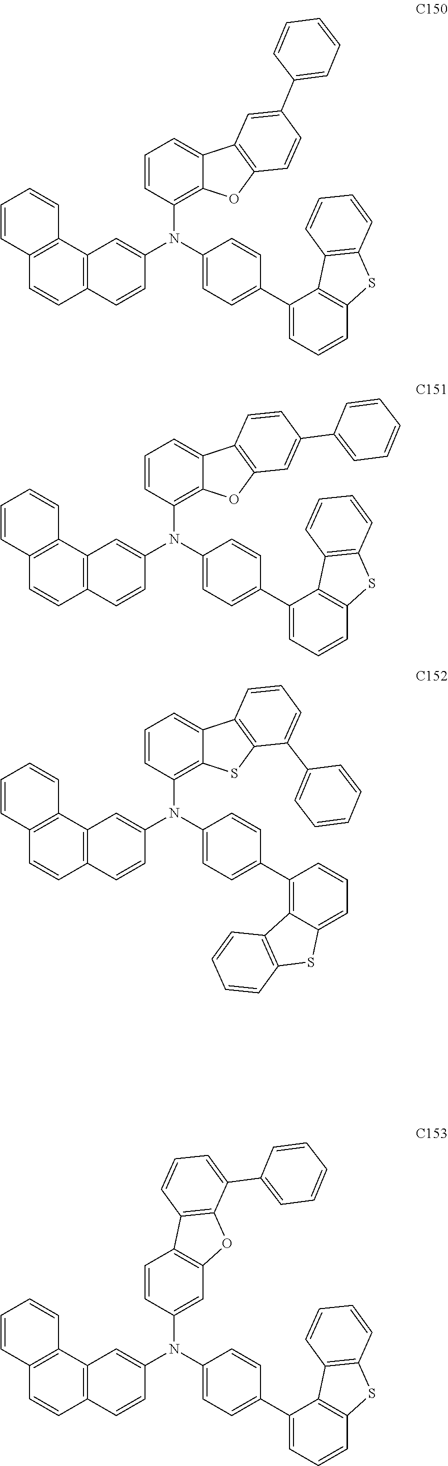 [SMILES: c1ccc(-c2ccc3oc4c(N(c5ccc(-c6cccc7sc8ccccc8c67)cc5)c5ccc6ccc7ccccc7c6c5)cccc4c3c2)cc1 ; c1ccc(-c2ccc3c(c2)oc2c(N(c4ccc(-c5cccc6sc7ccccc7c56)cc4)c4ccc5ccc6ccccc6c5c4)cccc23)cc1 ; c1ccc(-c2cccc3c2sc2c(N(c4ccc(-c5cccc6sc7ccccc7c56)cc4)c4ccc5ccc6ccccc6c5c4)cccc23)cc1 ; c1ccc(-c2cccc3c2oc2cc(N(c4ccc(-c5cccc6sc7ccccc7c56)cc4)c4ccc5ccc6ccccc6c5c4)ccc23)cc1]

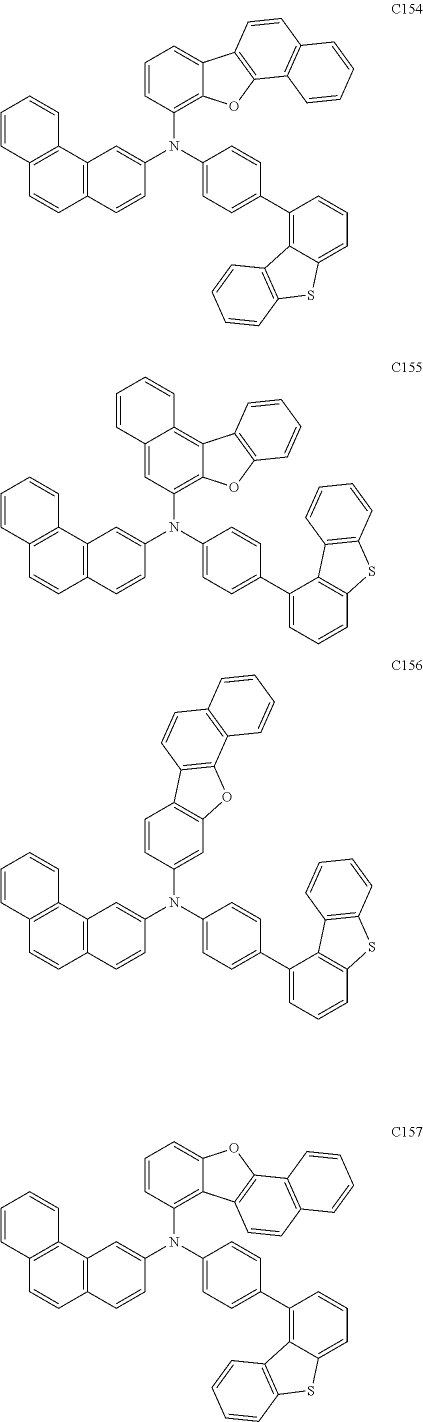 [SMILES: c1ccc2c(c1)ccc1ccc(N(c3ccc(-c4cccc5sc6ccccc6c45)cc3)c3cccc4c3oc3c5ccccc5ccc43)cc12 ; c1ccc2c(c1)ccc1ccc(N(c3ccc(-c4cccc5sc6ccccc6c45)cc3)c3cc4ccccc4c4c3oc3ccccc34)cc12 ; c1ccc2c(c1)ccc1ccc(N(c3ccc(-c4cccc5sc6ccccc6c45)cc3)c3ccc4c(c3)oc3c5ccccc5ccc43)cc12 ; c1ccc2c(c1)ccc1ccc(N(c3ccc(-c4cccc5sc6ccccc6c45)cc3)c3cccc4oc5c6ccccc6ccc5c34)cc12]

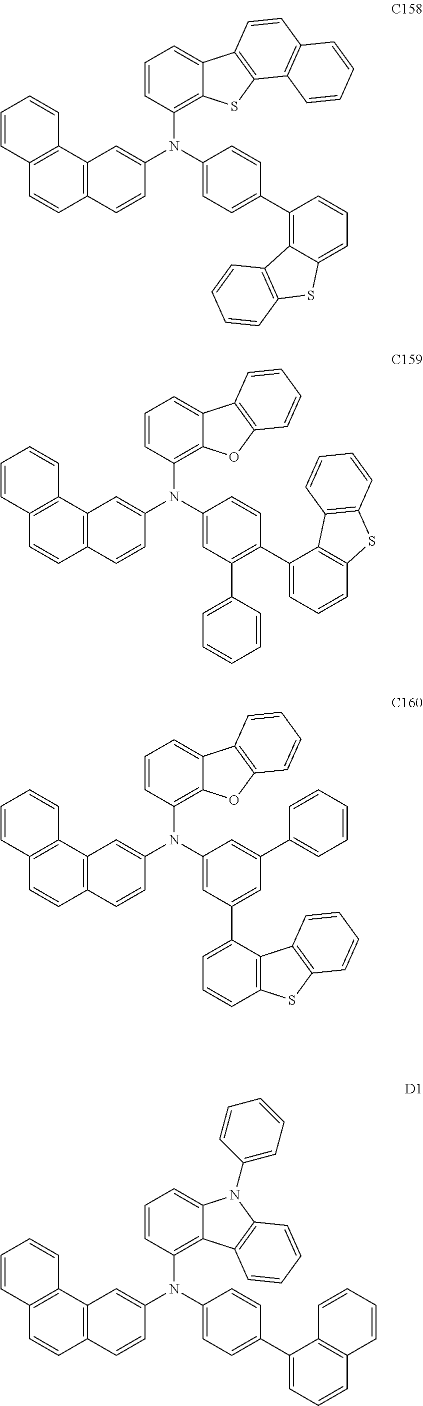 [SMILES: c1ccc2c(c1)ccc1ccc(N(c3ccc(-c4cccc5sc6ccccc6c45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2cccc3sc4ccccc4c23)cc1 ; c1ccc(-c2cc(-c3cccc4sc5ccccc5c34)cc(N(c3ccc4ccc5ccccc5c4c3)c3cccc4c3oc3ccccc34)c2)cc1 ; c1ccc(-n2c3ccccc3c3c(N(c4ccc(-c5cccc6ccccc56)cc4)c4ccc5ccc6ccccc6c5c4)cccc32)cc1]

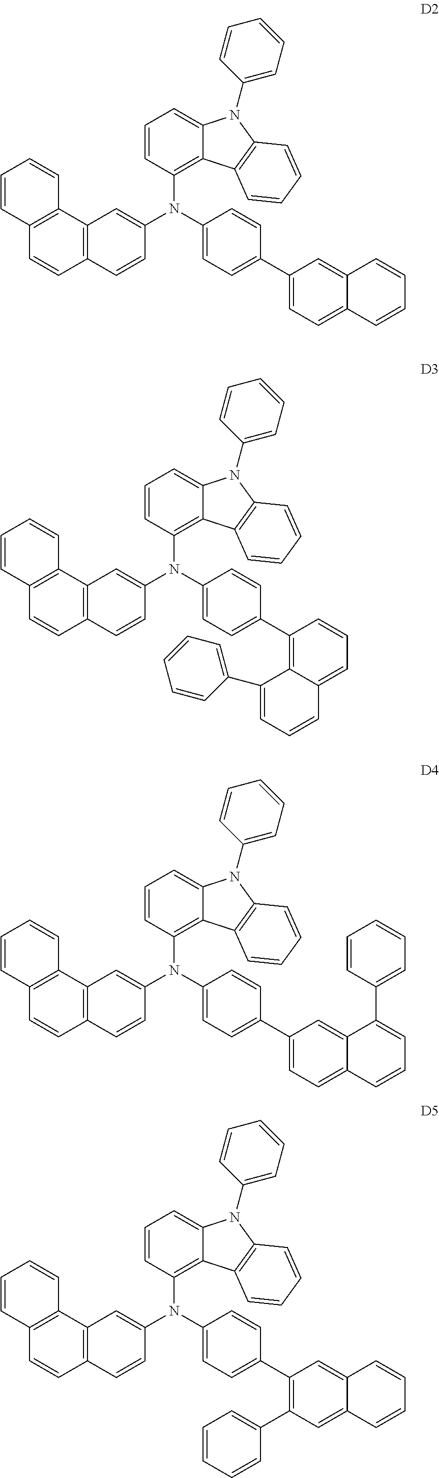 [SMILES: c1ccc(-n2c3ccccc3c3c(N(c4ccc(-c5ccc6ccccc6c5)cc4)c4ccc5ccc6ccccc6c5c4)cccc32)cc1 ; c1ccc(-c2cccc3cccc(-c4ccc(N(c5ccc6ccc7ccccc7c6c5)c5cccc6c5c5ccccc5n6-c5ccccc5)cc4)c23)cc1 ; c1ccc(-c2cccc3ccc(-c4ccc(N(c5ccc6ccc7ccccc7c6c5)c5cccc6c5c5ccccc5n6-c5ccccc5)cc4)cc23)cc1 ; c1ccc(-c2cc3ccccc3cc2-c2ccc(N(c3ccc4ccc5ccccc5c4c3)c3cccc4c3c3ccccc3n4-c3ccccc3)cc2)cc1]

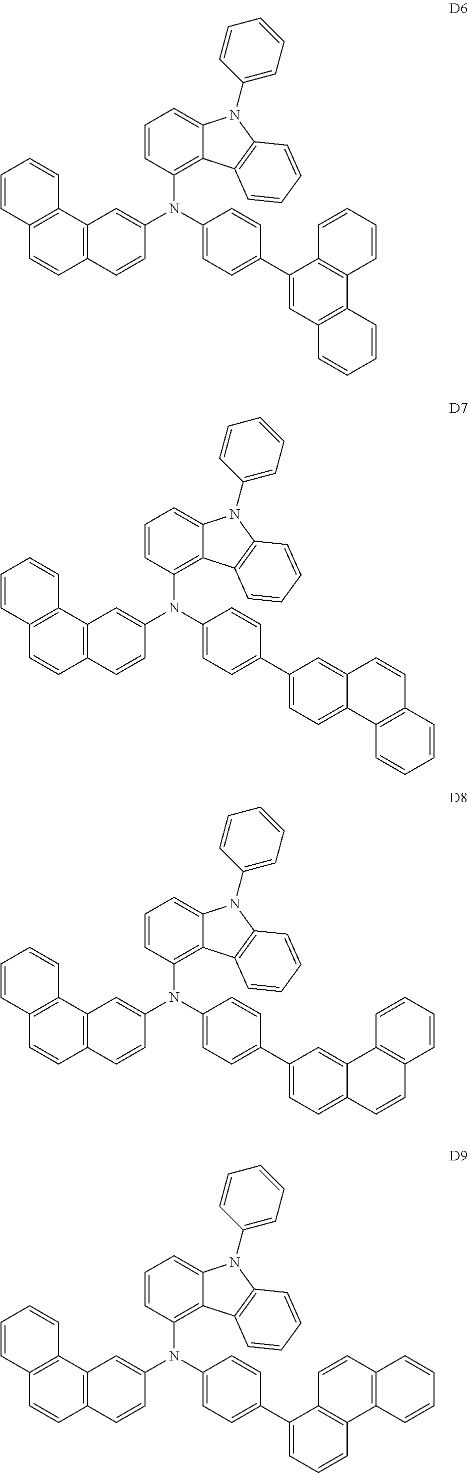 [SMILES: c1ccc(-n2c3ccccc3c3c(N(c4ccc(-c5cc6ccccc6c6ccccc56)cc4)c4ccc5ccc6ccccc6c5c4)cccc32)cc1 ; c1ccc(-n2c3ccccc3c3c(N(c4ccc(-c5ccc6c(ccc7ccccc76)c5)cc4)c4ccc5ccc6ccccc6c5c4)cccc32)cc1 ; c1ccc(-n2c3ccccc3c3c(N(c4ccc(-c5ccc6ccc7ccccc7c6c5)cc4)c4ccc5ccc6ccccc6c5c4)cccc32)cc1 ; c1ccc(-n2c3ccccc3c3c(N(c4ccc(-c5cccc6c5ccc5ccccc56)cc4)c4ccc5ccc6ccccc6c5c4)cccc32)cc1]

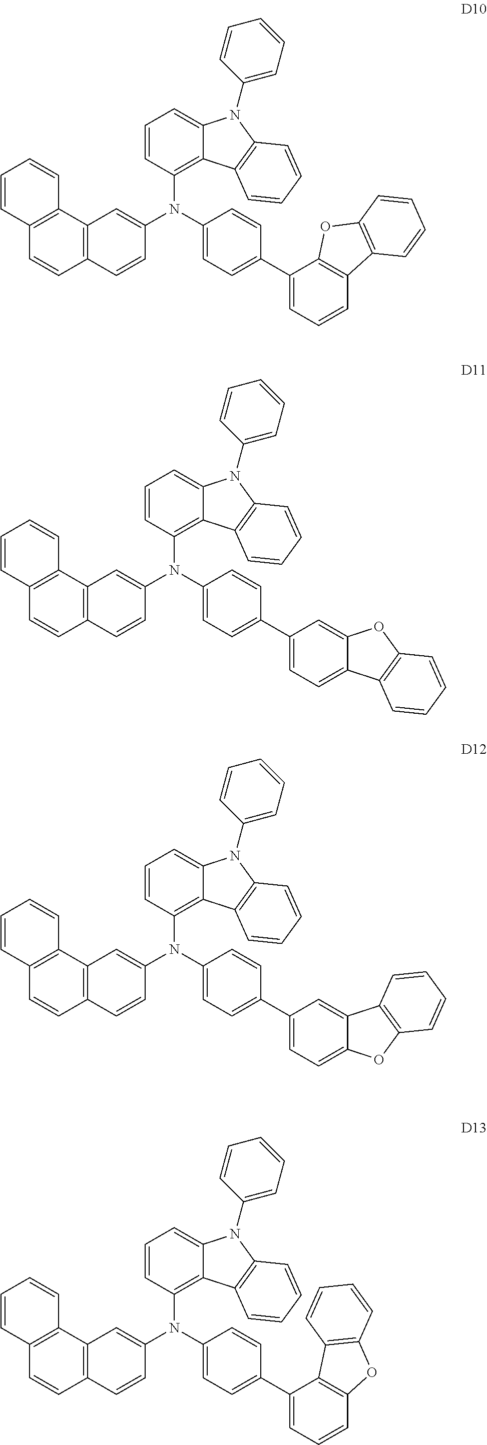 [SMILES: c1ccc(-n2c3ccccc3c3c(N(c4ccc(-c5cccc6c5oc5ccccc56)cc4)c4ccc5ccc6ccccc6c5c4)cccc32)cc1 ; c1ccc(-n2c3ccccc3c3c(N(c4ccc(-c5ccc6c(c5)oc5ccccc56)cc4)c4ccc5ccc6ccccc6c5c4)cccc32)cc1 ; c1ccc(-n2c3ccccc3c3c(N(c4ccc(-c5ccc6oc7ccccc7c6c5)cc4)c4ccc5ccc6ccccc6c5c4)cccc32)cc1 ; c1ccc(-n2c3ccccc3c3c(N(c4ccc(-c5cccc6oc7ccccc7c56)cc4)c4ccc5ccc6ccccc6c5c4)cccc32)cc1]

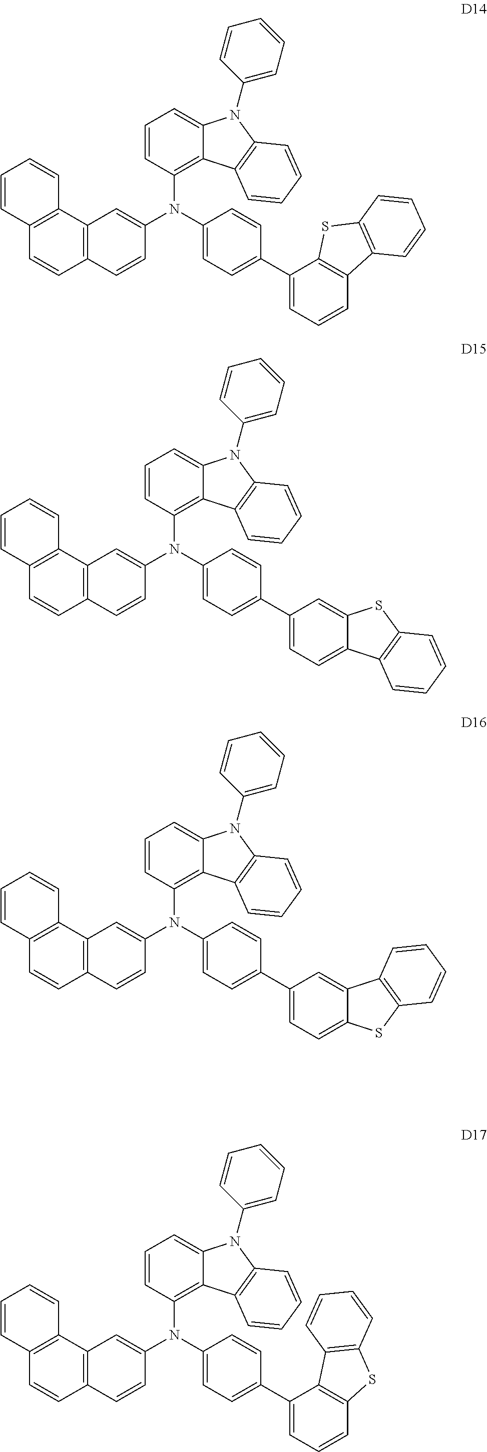 [SMILES: c1ccc(-n2c3ccccc3c3c(N(c4ccc(-c5cccc6c5sc5ccccc56)cc4)c4ccc5ccc6ccccc6c5c4)cccc32)cc1 ; c1ccc(-n2c3ccccc3c3c(N(c4ccc(-c5ccc6c(c5)sc5ccccc56)cc4)c4ccc5ccc6ccccc6c5c4)cccc32)cc1 ; c1ccc(-n2c3ccccc3c3c(N(c4ccc(-c5ccc6sc7ccccc7c6c5)cc4)c4ccc5ccc6ccccc6c5c4)cccc32)cc1 ; c1ccc(-n2c3ccccc3c3c(N(c4ccc(-c5cccc6sc7ccccc7c56)cc4)c4ccc5ccc6ccccc6c5c4)cccc32)cc1]

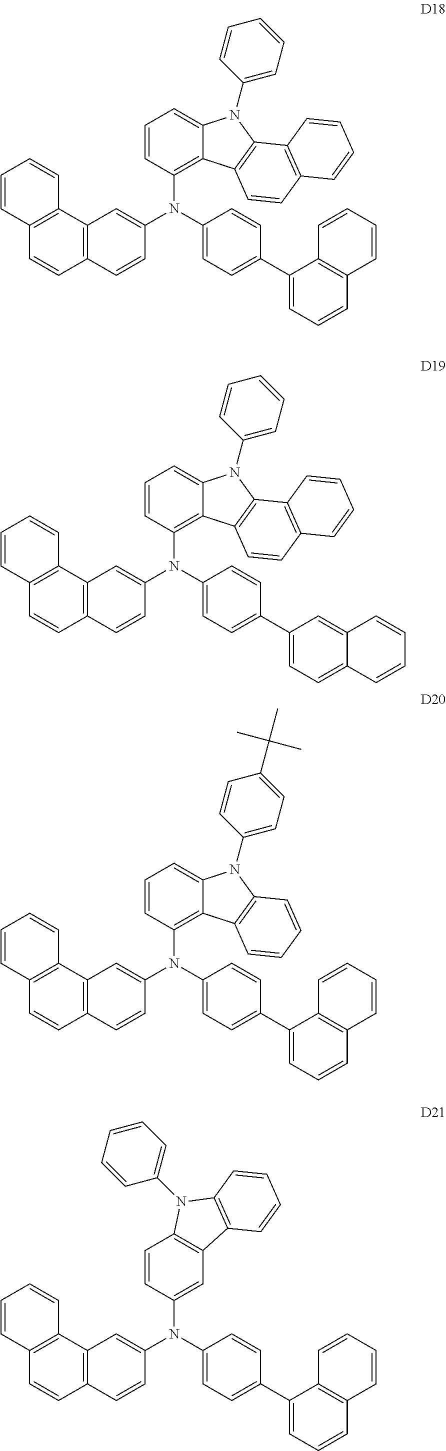

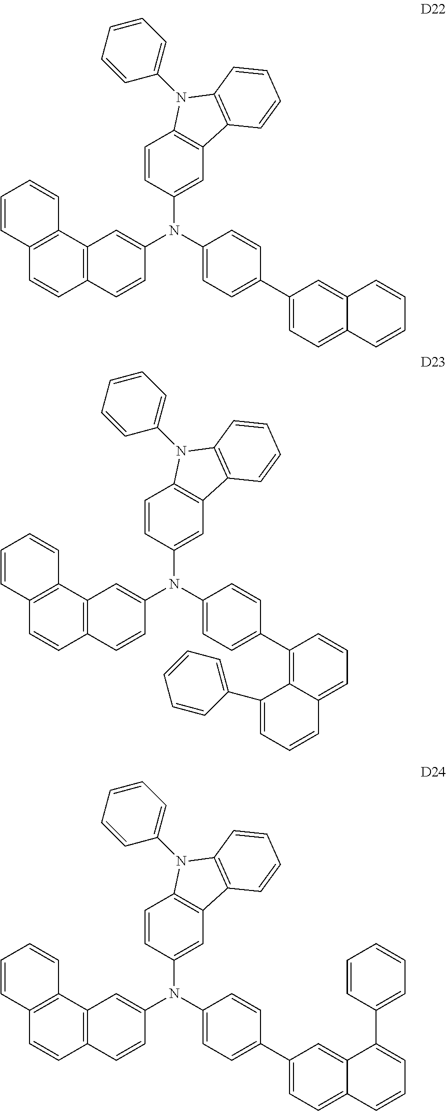 [SMILES: c1ccc(-n2c3ccccc3c3cc(N(c4ccc(-c5ccc6ccccc6c5)cc4)c4ccc5ccc6ccccc6c5c4)ccc32)cc1 ; c1ccc(-c2cccc3cccc(-c4ccc(N(c5ccc6ccc7ccccc7c6c5)c5ccc6c(c5)c5ccccc5n6-c5ccccc5)cc4)c23)cc1 ; c1ccc(-c2cccc3ccc(-c4ccc(N(c5ccc6ccc7ccccc7c6c5)c5ccc6c(c5)c5ccccc5n6-c5ccccc5)cc4)cc23)cc1]

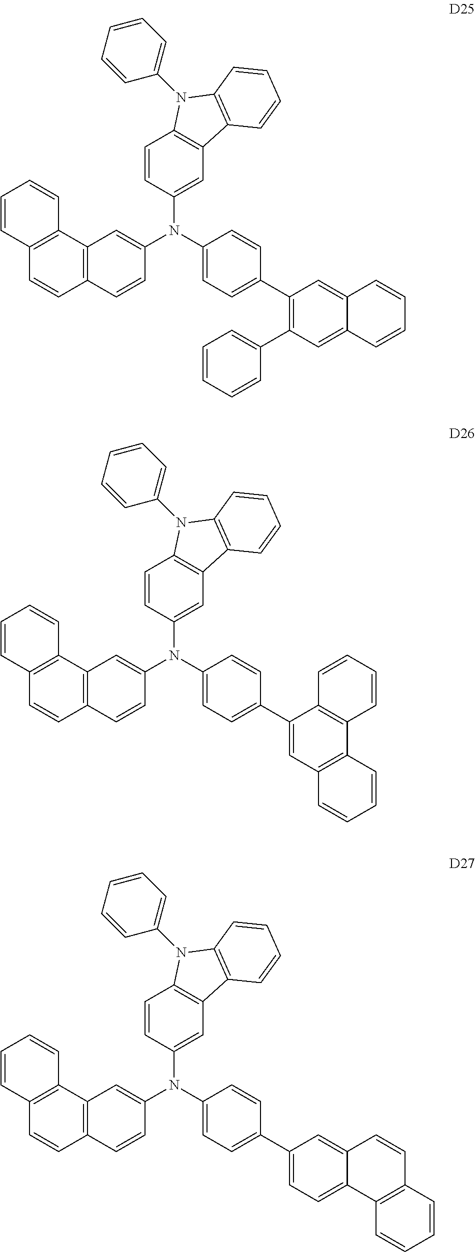 [SMILES: c1ccc(-c2cc3ccccc3cc2-c2ccc(N(c3ccc4ccc5ccccc5c4c3)c3ccc4c(c3)c3ccccc3n4-c3ccccc3)cc2)cc1 ; c1ccc(-n2c3ccccc3c3cc(N(c4ccc(-c5cc6ccccc6c6ccccc56)cc4)c4ccc5ccc6ccccc6c5c4)ccc32)cc1 ; c1ccc(-n2c3ccccc3c3cc(N(c4ccc(-c5ccc6c(ccc7ccccc76)c5)cc4)c4ccc5ccc6ccccc6c5c4)ccc32)cc1]

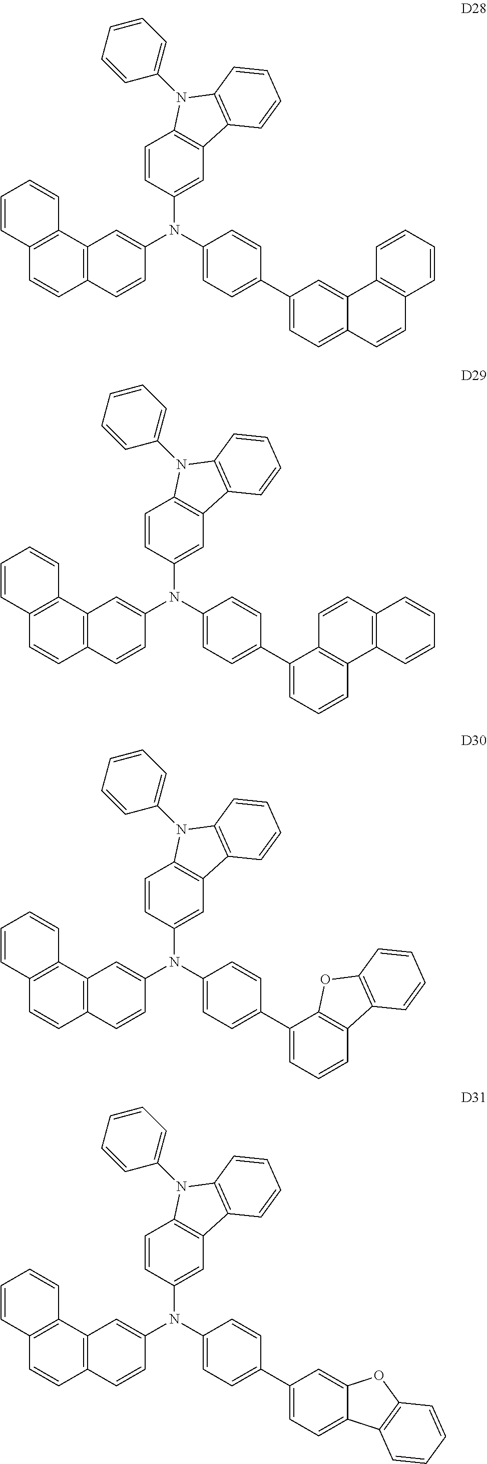 [SMILES: c1ccc(-n2c3ccccc3c3cc(N(c4ccc(-c5ccc6ccc7ccccc7c6c5)cc4)c4ccc5ccc6ccccc6c5c4)ccc32)cc1 ; c1ccc(-n2c3ccccc3c3cc(N(c4ccc(-c5cccc6c5ccc5ccccc56)cc4)c4ccc5ccc6ccccc6c5c4)ccc32)cc1 ; c1ccc(-n2c3ccccc3c3cc(N(c4ccc(-c5cccc6c5oc5ccccc56)cc4)c4ccc5ccc6ccccc6c5c4)ccc32)cc1 ; c1ccc(-n2c3ccccc3c3cc(N(c4ccc(-c5ccc6c(c5)oc5ccccc56)cc4)c4ccc5ccc6ccccc6c5c4)ccc32)cc1]

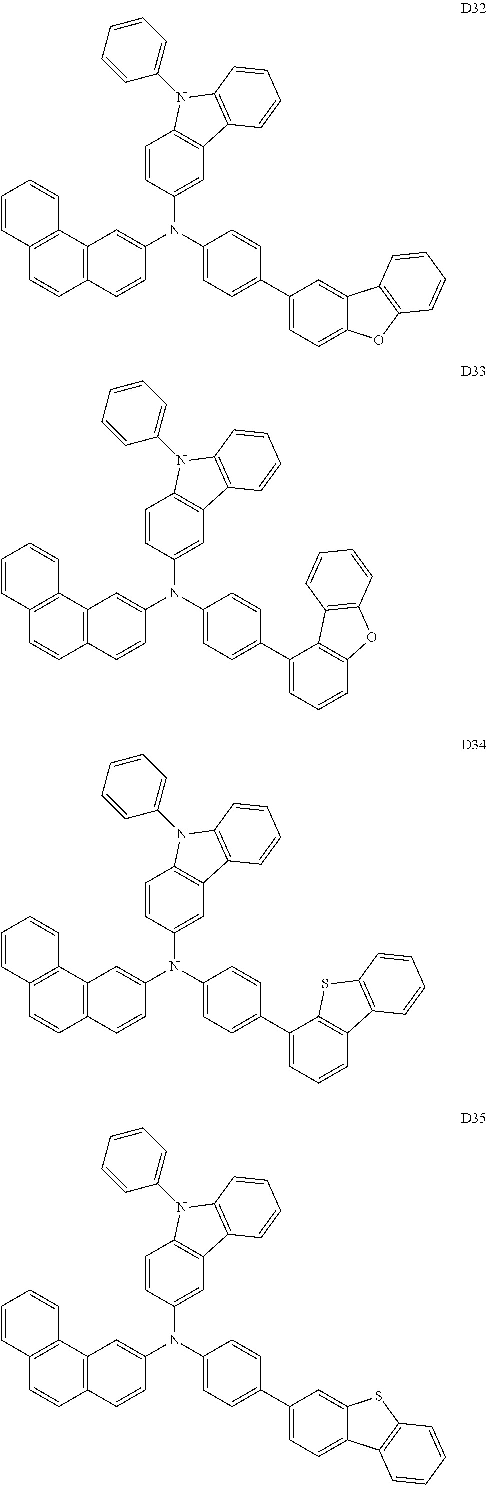 [SMILES: c1ccc(-n2c3ccccc3c3cc(N(c4ccc(-c5ccc6oc7ccccc7c6c5)cc4)c4ccc5ccc6ccccc6c5c4)ccc32)cc1 ; c1ccc(-n2c3ccccc3c3cc(N(c4ccc(-c5cccc6oc7ccccc7c56)cc4)c4ccc5ccc6ccccc6c5c4)ccc32)cc1 ; c1ccc(-n2c3ccccc3c3cc(N(c4ccc(-c5cccc6c5sc5ccccc56)cc4)c4ccc5ccc6ccccc6c5c4)ccc32)cc1 ; c1ccc(-n2c3ccccc3c3cc(N(c4ccc(-c5ccc6c(c5)sc5ccccc56)cc4)c4ccc5ccc6ccccc6c5c4)ccc32)cc1]

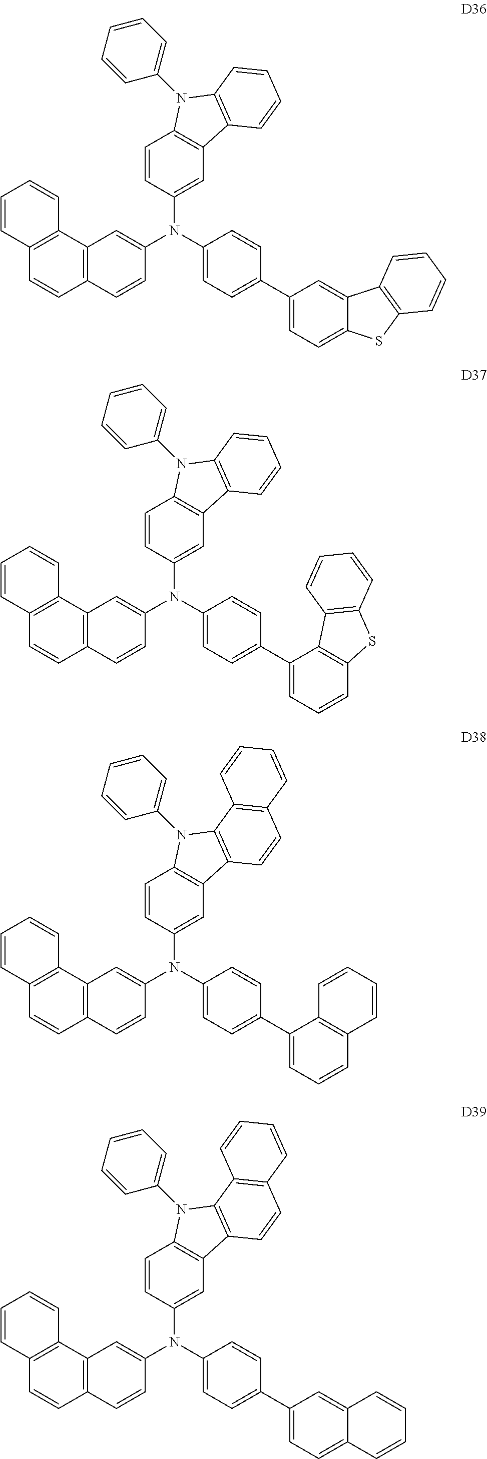 [SMILES: c1ccc(-n2c3ccccc3c3cc(N(c4ccc(-c5ccc6sc7ccccc7c6c5)cc4)c4ccc5ccc6ccccc6c5c4)ccc32)cc1 ; c1ccc(-n2c3ccccc3c3cc(N(c4ccc(-c5cccc6sc7ccccc7c56)cc4)c4ccc5ccc6ccccc6c5c4)ccc32)cc1 ; c1ccc(-n2c3ccc(N(c4ccc(-c5cccc6ccccc56)cc4)c4ccc5ccc6ccccc6c5c4)cc3c3ccc4ccccc4c32)cc1 ; c1ccc(-n2c3ccc(N(c4ccc(-c5ccc6ccccc6c5)cc4)c4ccc5ccc6ccccc6c5c4)cc3c3ccc4ccccc4c32)cc1]

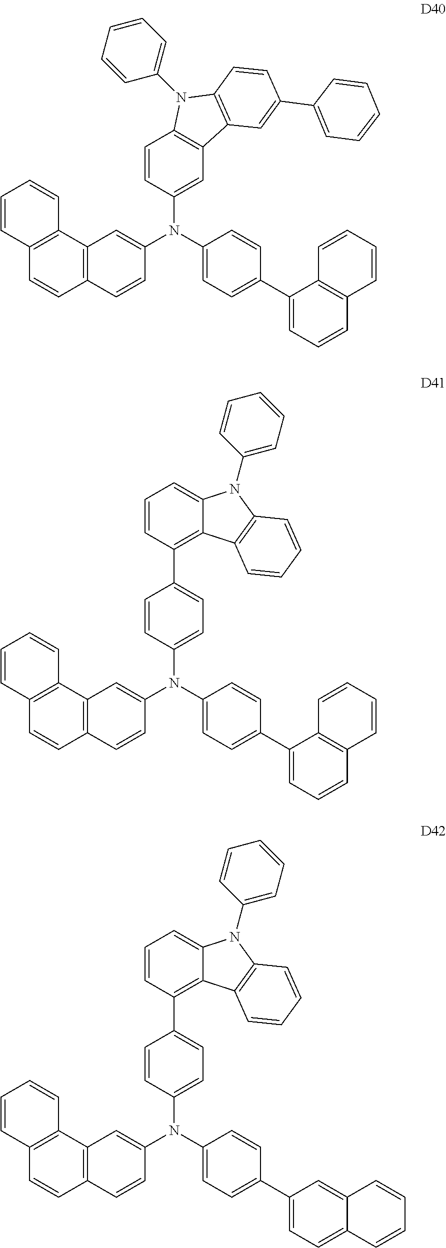 [SMILES: c1ccc(-c2ccc3c(c2)c2cc(N(c4ccc(-c5cccc6ccccc56)cc4)c4ccc5ccc6ccccc6c5c4)ccc2n3-c2ccccc2)cc1 ; c1ccc(-n2c3ccccc3c3c(-c4ccc(N(c5ccc(-c6cccc7ccccc67)cc5)c5ccc6ccc7ccccc7c6c5)cc4)cccc32)cc1 ; c1ccc(-n2c3ccccc3c3c(-c4ccc(N(c5ccc(-c6ccc7ccccc7c6)cc5)c5ccc6ccc7ccccc7c6c5)cc4)cccc32)cc1]

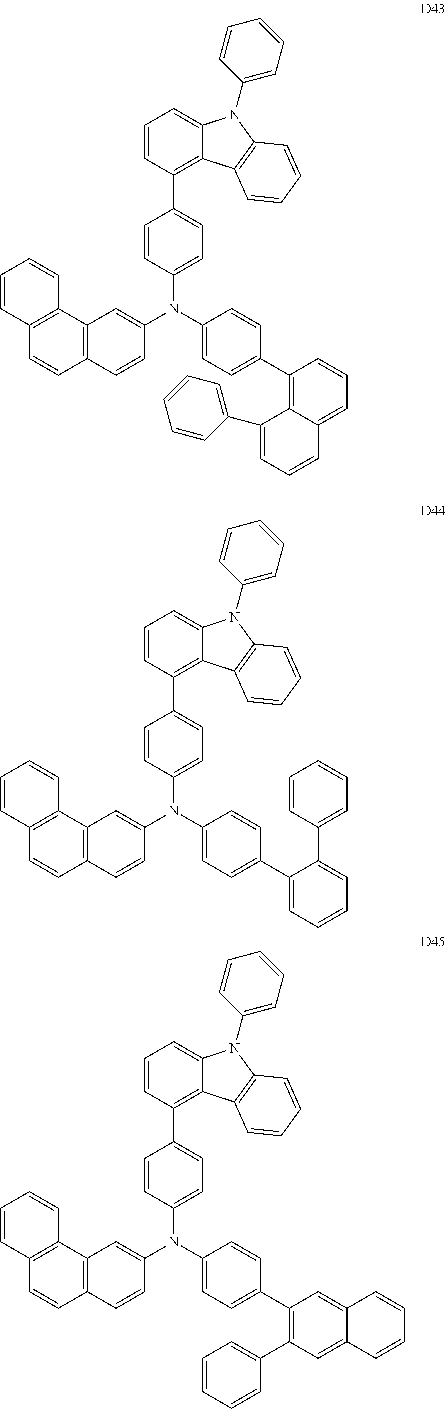 [SMILES: c1ccc(-c2cccc3cccc(-c4ccc(N(c5ccc(-c6cccc7c6c6ccccc6n7-c6ccccc6)cc5)c5ccc6ccc7ccccc7c6c5)cc4)c23)cc1 ; c1ccc(-c2ccccc2-c2ccc(N(c3ccc(-c4cccc5c4c4ccccc4n5-c4ccccc4)cc3)c3ccc4ccc5ccccc5c4c3)cc2)cc1 ; c1ccc(-c2cc3ccccc3cc2-c2ccc(N(c3ccc(-c4cccc5c4c4ccccc4n5-c4ccccc4)cc3)c3ccc4ccc5ccccc5c4c3)cc2)cc1]

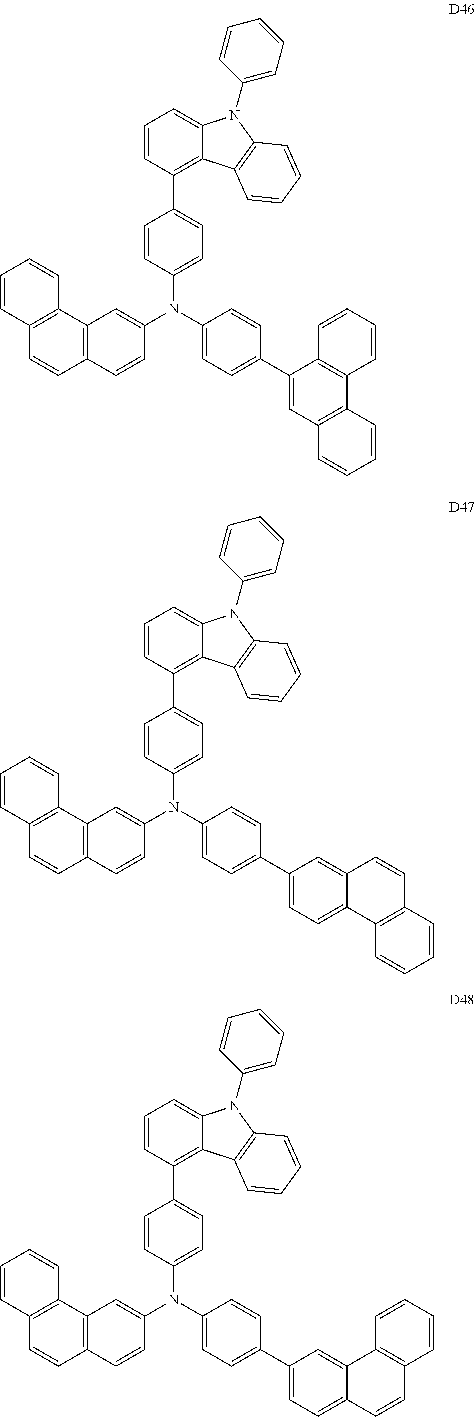 [SMILES: c1ccc(-n2c3ccccc3c3c(-c4ccc(N(c5ccc(-c6cc7ccccc7c7ccccc67)cc5)c5ccc6ccc7ccccc7c6c5)cc4)cccc32)cc1 ; c1ccc(-n2c3ccccc3c3c(-c4ccc(N(c5ccc(-c6ccc7c(ccc8ccccc87)c6)cc5)c5ccc6ccc7ccccc7c6c5)cc4)cccc32)cc1 ; c1ccc(-n2c3ccccc3c3c(-c4ccc(N(c5ccc(-c6ccc7ccc8ccccc8c7c6)cc5)c5ccc6ccc7ccccc7c6c5)cc4)cccc32)cc1]

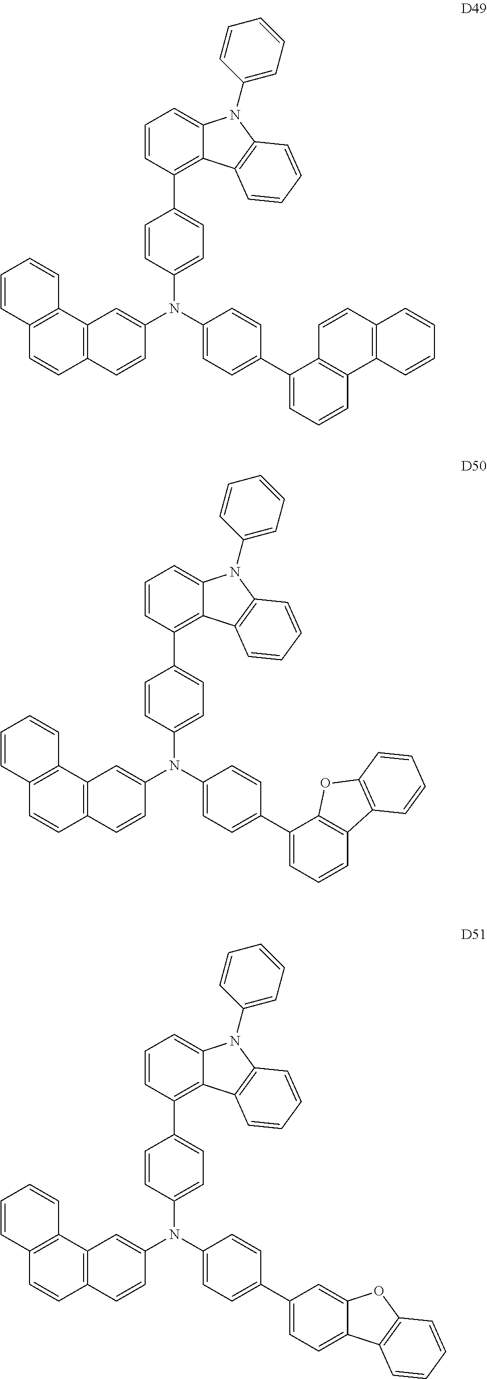 [SMILES: c1ccc(-n2c3ccccc3c3c(-c4ccc(N(c5ccc(-c6cccc7c6ccc6ccccc67)cc5)c5ccc6ccc7ccccc7c6c5)cc4)cccc32)cc1 ; c1ccc(-n2c3ccccc3c3c(-c4ccc(N(c5ccc(-c6cccc7c6oc6ccccc67)cc5)c5ccc6ccc7ccccc7c6c5)cc4)cccc32)cc1 ; c1ccc(-n2c3ccccc3c3c(-c4ccc(N(c5ccc(-c6ccc7c(c6)oc6ccccc67)cc5)c5ccc6ccc7ccccc7c6c5)cc4)cccc32)cc1]

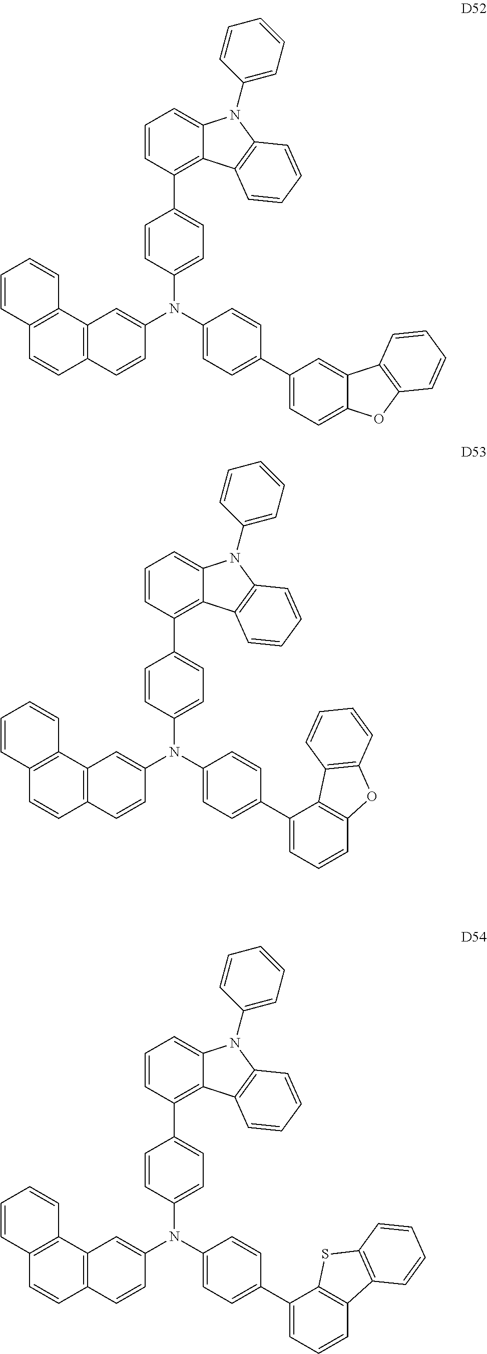 [SMILES: c1ccc(-n2c3ccccc3c3c(-c4ccc(N(c5ccc(-c6ccc7oc8ccccc8c7c6)cc5)c5ccc6ccc7ccccc7c6c5)cc4)cccc32)cc1 ; c1ccc(-n2c3ccccc3c3c(-c4ccc(N(c5ccc(-c6cccc7oc8ccccc8c67)cc5)c5ccc6ccc7ccccc7c6c5)cc4)cccc32)cc1 ; c1ccc(-n2c3ccccc3c3c(-c4ccc(N(c5ccc(-c6cccc7c6sc6ccccc67)cc5)c5ccc6ccc7ccccc7c6c5)cc4)cccc32)cc1]

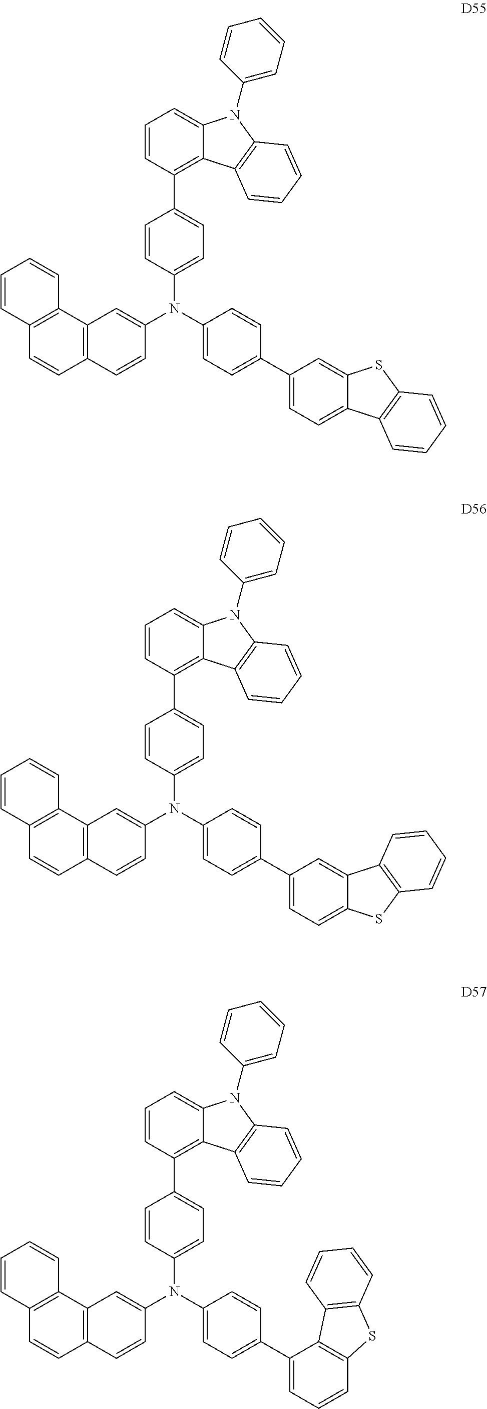 [SMILES: c1ccc(-n2c3ccccc3c3c(-c4ccc(N(c5ccc(-c6ccc7c(c6)sc6ccccc67)cc5)c5ccc6ccc7ccccc7c6c5)cc4)cccc32)cc1 ; c1ccc(-n2c3ccccc3c3c(-c4ccc(N(c5ccc(-c6ccc7sc8ccccc8c7c6)cc5)c5ccc6ccc7ccccc7c6c5)cc4)cccc32)cc1 ; c1ccc(-n2c3ccccc3c3c(-c4ccc(N(c5ccc(-c6cccc7sc8ccccc8c67)cc5)c5ccc6ccc7ccccc7c6c5)cc4)cccc32)cc1]

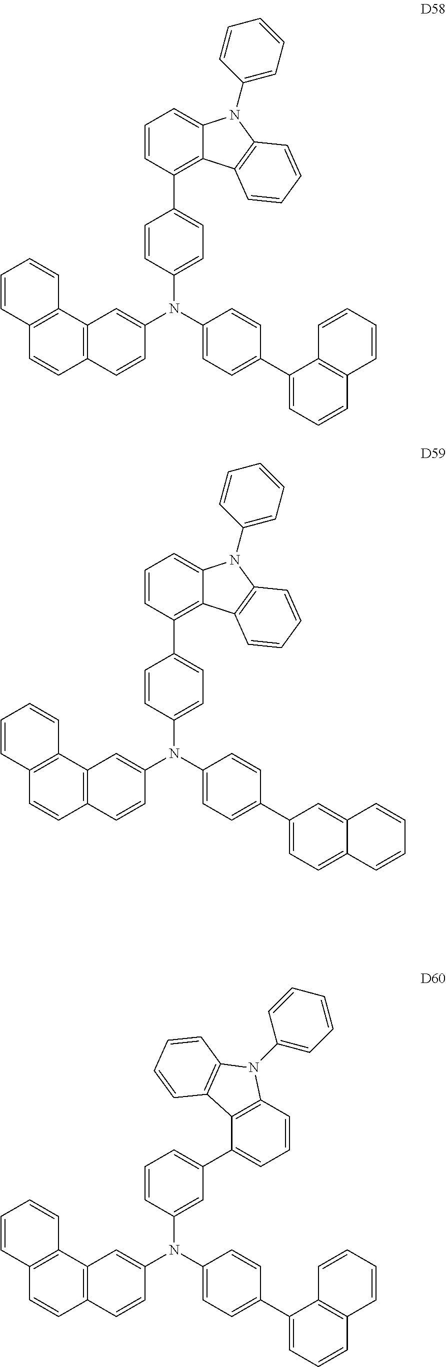 [SMILES: c1ccc(-n2c3ccccc3c3c(-c4ccc(N(c5ccc(-c6cccc7ccccc67)cc5)c5ccc6ccc7ccccc7c6c5)cc4)cccc32)cc1 ; c1ccc(-n2c3ccccc3c3c(-c4ccc(N(c5ccc(-c6ccc7ccccc7c6)cc5)c5ccc6ccc7ccccc7c6c5)cc4)cccc32)cc1 ; c1ccc(-n2c3ccccc3c3c(-c4cccc(N(c5ccc(-c6cccc7ccccc67)cc5)c5ccc6ccc7ccccc7c6c5)c4)cccc32)cc1]

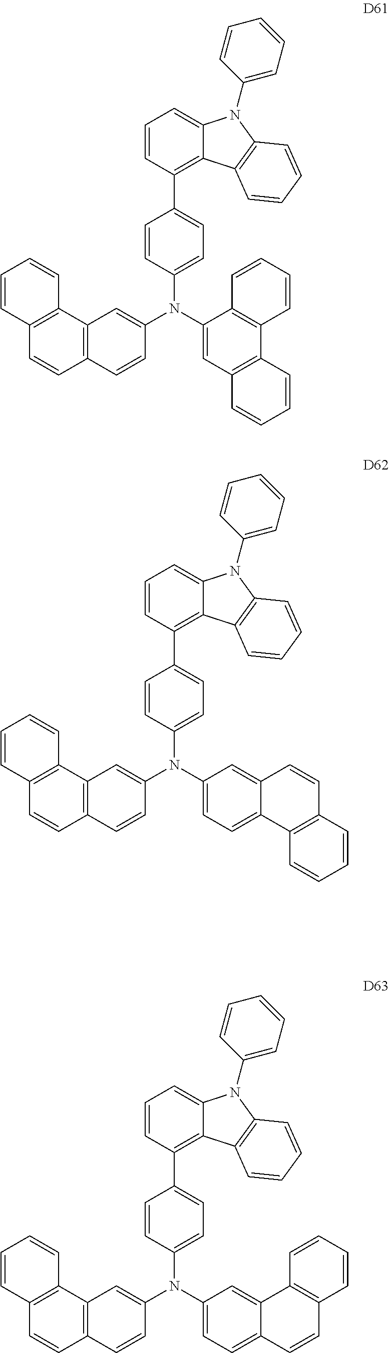 [SMILES: c1ccc(-n2c3ccccc3c3c(-c4ccc(N(c5ccc6ccc7ccccc7c6c5)c5cc6ccccc6c6ccccc56)cc4)cccc32)cc1 ; c1ccc(-n2c3ccccc3c3c(-c4ccc(N(c5ccc6c(ccc7ccccc76)c5)c5ccc6ccc7ccccc7c6c5)cc4)cccc32)cc1 ; c1ccc(-n2c3ccccc3c3c(-c4ccc(N(c5ccc6ccc7ccccc7c6c5)c5ccc6ccc7ccccc7c6c5)cc4)cccc32)cc1]

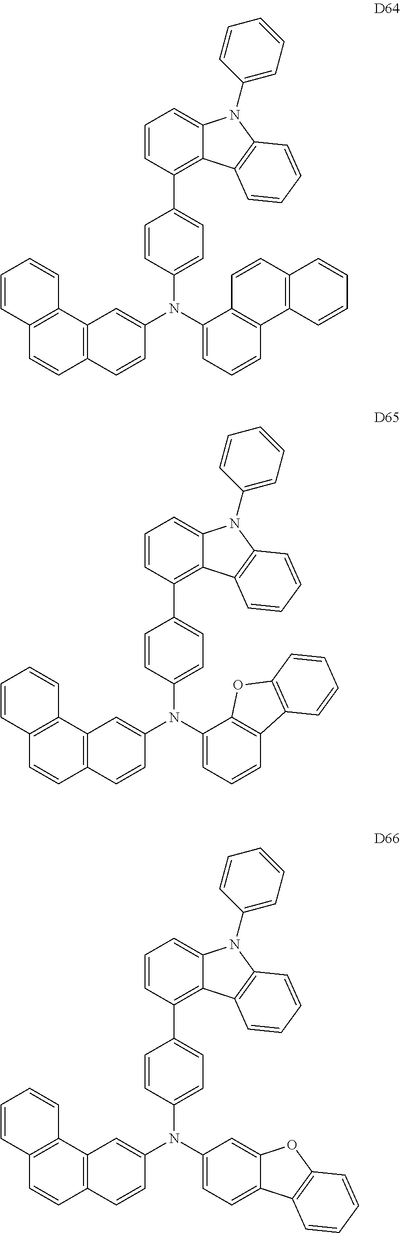 [SMILES: c1ccc(-n2c3ccccc3c3c(-c4ccc(N(c5ccc6ccc7ccccc7c6c5)c5cccc6c5ccc5ccccc56)cc4)cccc32)cc1 ; c1ccc(-n2c3ccccc3c3c(-c4ccc(N(c5ccc6ccc7ccccc7c6c5)c5cccc6c5oc5ccccc56)cc4)cccc32)cc1 ; c1ccc(-n2c3ccccc3c3c(-c4ccc(N(c5ccc6c(c5)oc5ccccc56)c5ccc6ccc7ccccc7c6c5)cc4)cccc32)cc1]

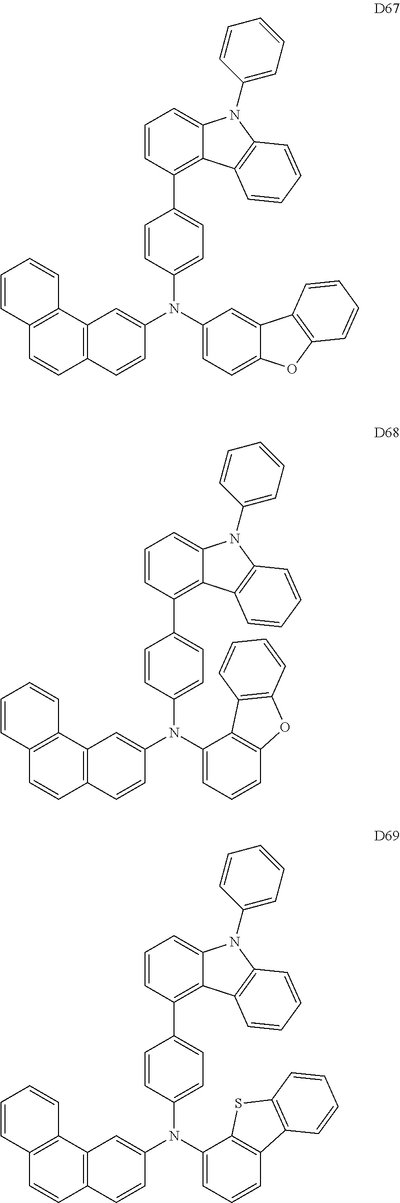 [SMILES: c1ccc(-n2c3ccccc3c3c(-c4ccc(N(c5ccc6ccc7ccccc7c6c5)c5ccc6oc7ccccc7c6c5)cc4)cccc32)cc1 ; c1ccc(-n2c3ccccc3c3c(-c4ccc(N(c5ccc6ccc7ccccc7c6c5)c5cccc6oc7ccccc7c56)cc4)cccc32)cc1 ; c1ccc(-n2c3ccccc3c3c(-c4ccc(N(c5ccc6ccc7ccccc7c6c5)c5cccc6c5sc5ccccc56)cc4)cccc32)cc1]

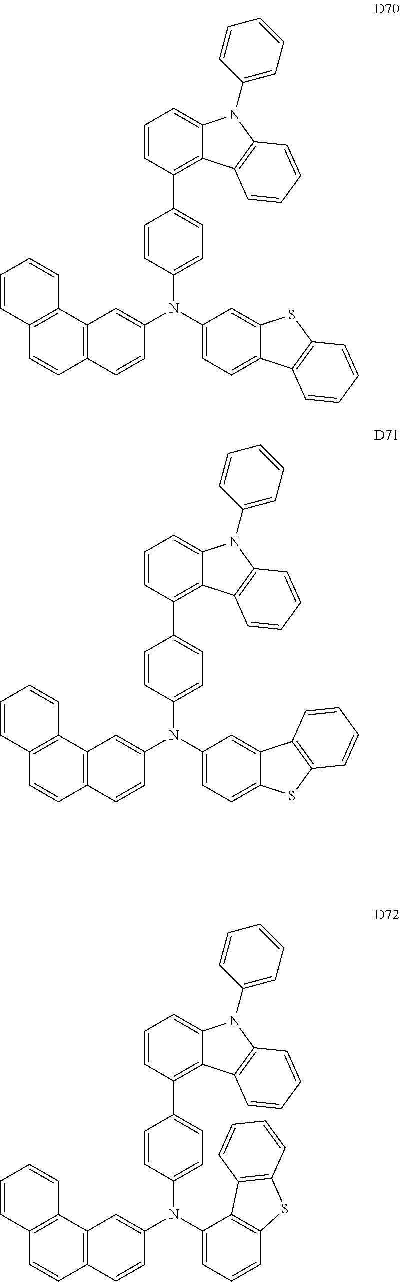 [SMILES: c1ccc(-n2c3ccccc3c3c(-c4ccc(N(c5ccc6c(c5)sc5ccccc56)c5ccc6ccc7ccccc7c6c5)cc4)cccc32)cc1 ; c1ccc(-n2c3ccccc3c3c(-c4ccc(N(c5ccc6ccc7ccccc7c6c5)c5ccc6sc7ccccc7c6c5)cc4)cccc32)cc1 ; c1ccc(-n2c3ccccc3c3c(-c4ccc(N(c5ccc6ccc7ccccc7c6c5)c5cccc6sc7ccccc7c56)cc4)cccc32)cc1]

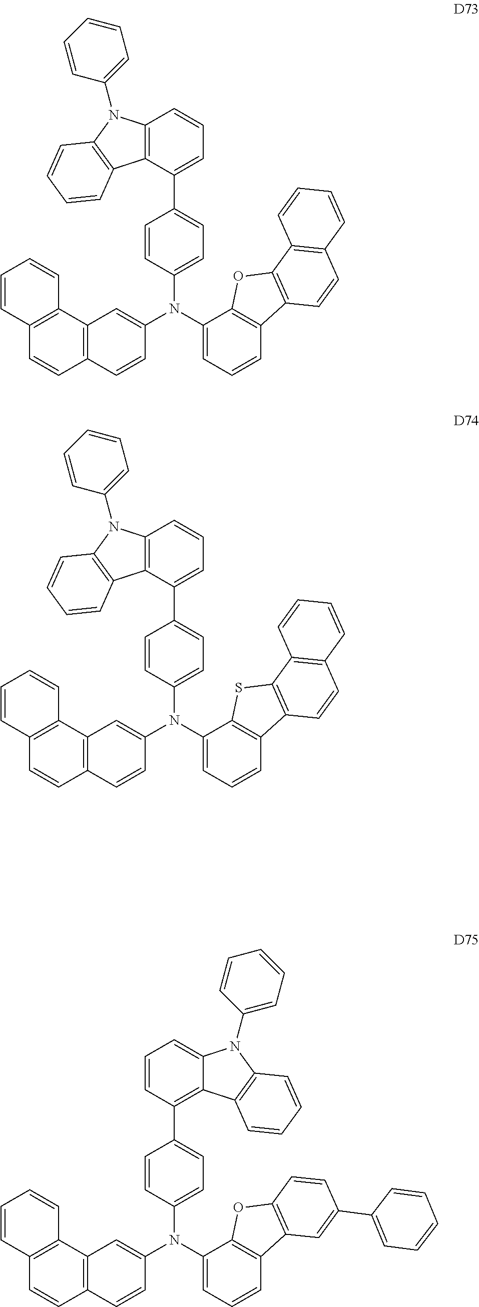 [SMILES: c1ccc(-n2c3ccccc3c3c(-c4ccc(N(c5ccc6ccc7ccccc7c6c5)c5cccc6c5oc5c7ccccc7ccc65)cc4)cccc32)cc1 ; c1ccc(-n2c3ccccc3c3c(-c4ccc(N(c5ccc6ccc7ccccc7c6c5)c5cccc6c5sc5c7ccccc7ccc65)cc4)cccc32)cc1 ; c1ccc(-c2ccc3oc4c(N(c5ccc(-c6cccc7c6c6ccccc6n7-c6ccccc6)cc5)c5ccc6ccc7ccccc7c6c5)cccc4c3c2)cc1]

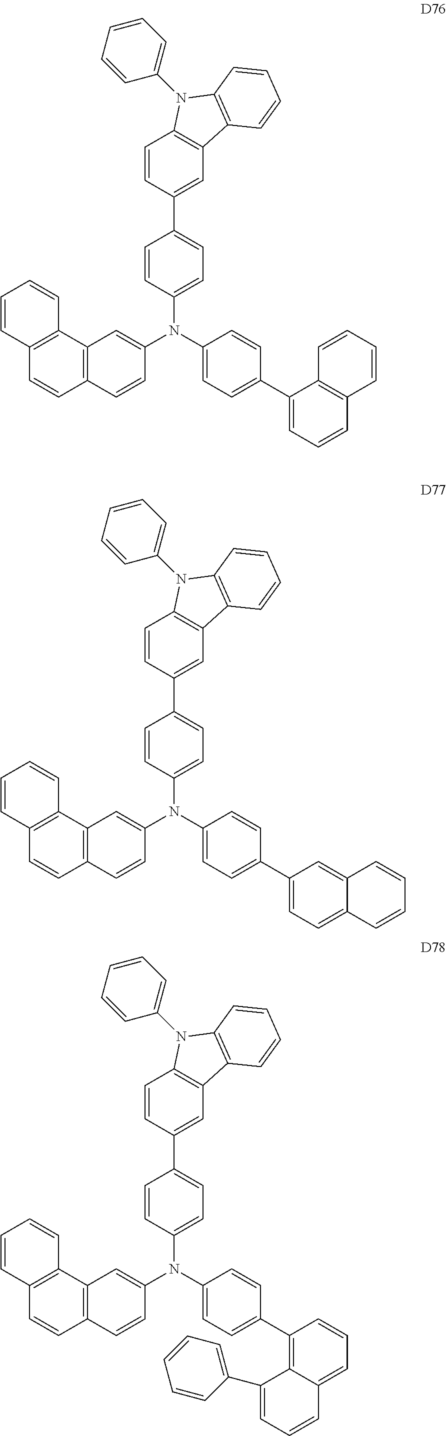 [SMILES: c1ccc(-n2c3ccccc3c3cc(-c4ccc(N(c5ccc(-c6cccc7ccccc67)cc5)c5ccc6ccc7ccccc7c6c5)cc4)ccc32)cc1 ; c1ccc(-n2c3ccccc3c3cc(-c4ccc(N(c5ccc(-c6ccc7ccccc7c6)cc5)c5ccc6ccc7ccccc7c6c5)cc4)ccc32)cc1 ; c1ccc(-c2cccc3cccc(-c4ccc(N(c5ccc(-c6ccc7c(c6)c6ccccc6n7-c6ccccc6)cc5)c5ccc6ccc7ccccc7c6c5)cc4)c23)cc1]

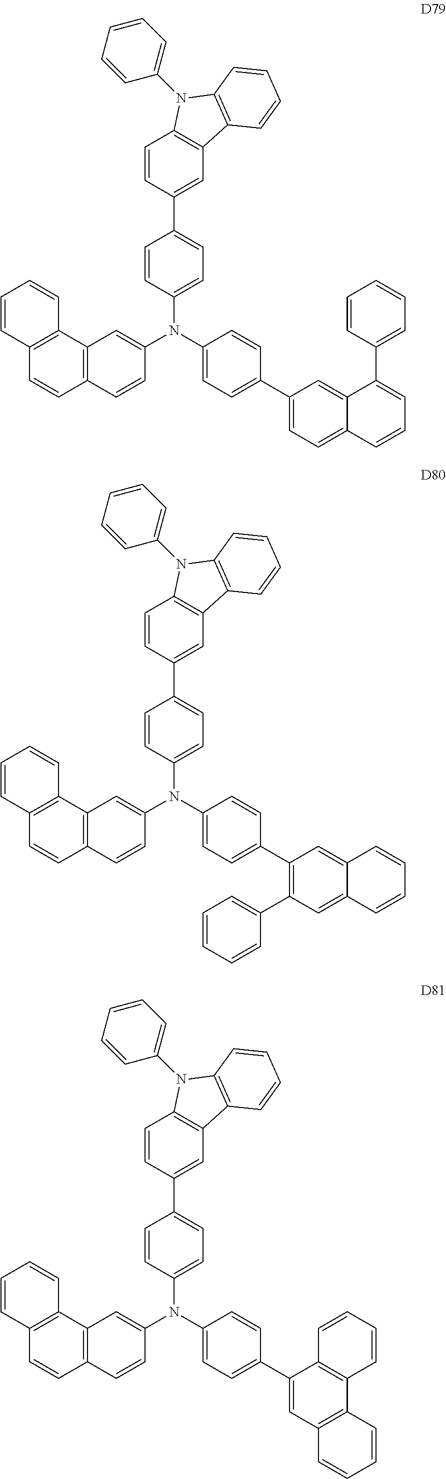 [SMILES: c1ccc(-c2cccc3ccc(-c4ccc(N(c5ccc(-c6ccc7c(c6)c6ccccc6n7-c6ccccc6)cc5)c5ccc6ccc7ccccc7c6c5)cc4)cc23)cc1 ; c1ccc(-c2cc3ccccc3cc2-c2ccc(N(c3ccc(-c4ccc5c(c4)c4ccccc4n5-c4ccccc4)cc3)c3ccc4ccc5ccccc5c4c3)cc2)cc1 ; c1ccc(-n2c3ccccc3c3cc(-c4ccc(N(c5ccc(-c6cc7ccccc7c7ccccc67)cc5)c5ccc6ccc7ccccc7c6c5)cc4)ccc32)cc1]

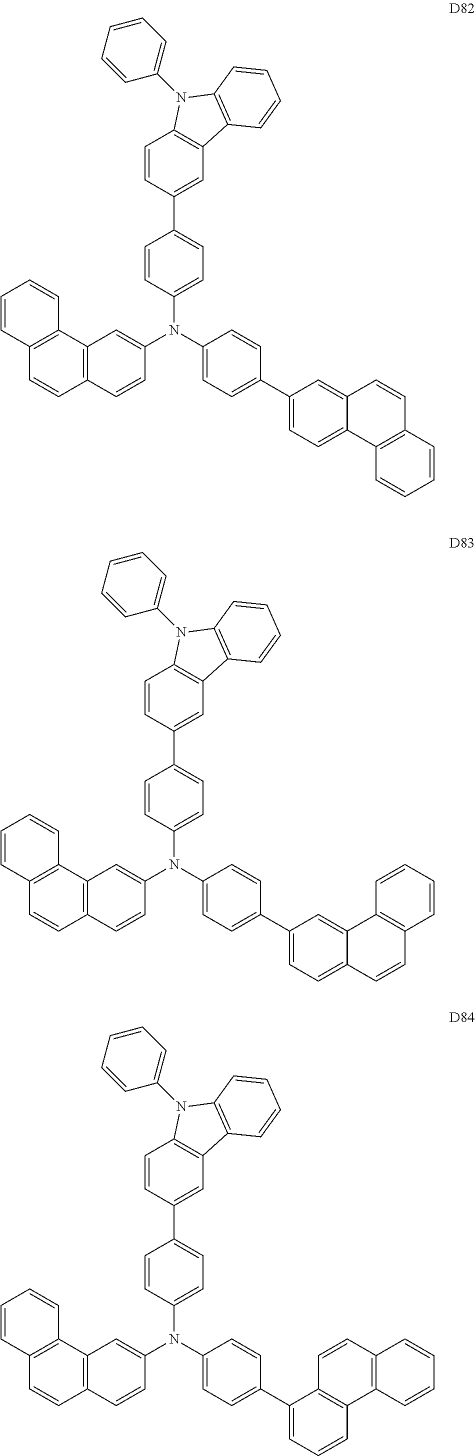 [SMILES: c1ccc(-n2c3ccccc3c3cc(-c4ccc(N(c5ccc(-c6ccc7c(ccc8ccccc87)c6)cc5)c5ccc6ccc7ccccc7c6c5)cc4)ccc32)cc1 ; c1ccc(-n2c3ccccc3c3cc(-c4ccc(N(c5ccc(-c6ccc7ccc8ccccc8c7c6)cc5)c5ccc6ccc7ccccc7c6c5)cc4)ccc32)cc1 ; c1ccc(-n2c3ccccc3c3cc(-c4ccc(N(c5ccc(-c6cccc7c6ccc6ccccc67)cc5)c5ccc6ccc7ccccc7c6c5)cc4)ccc32)cc1]

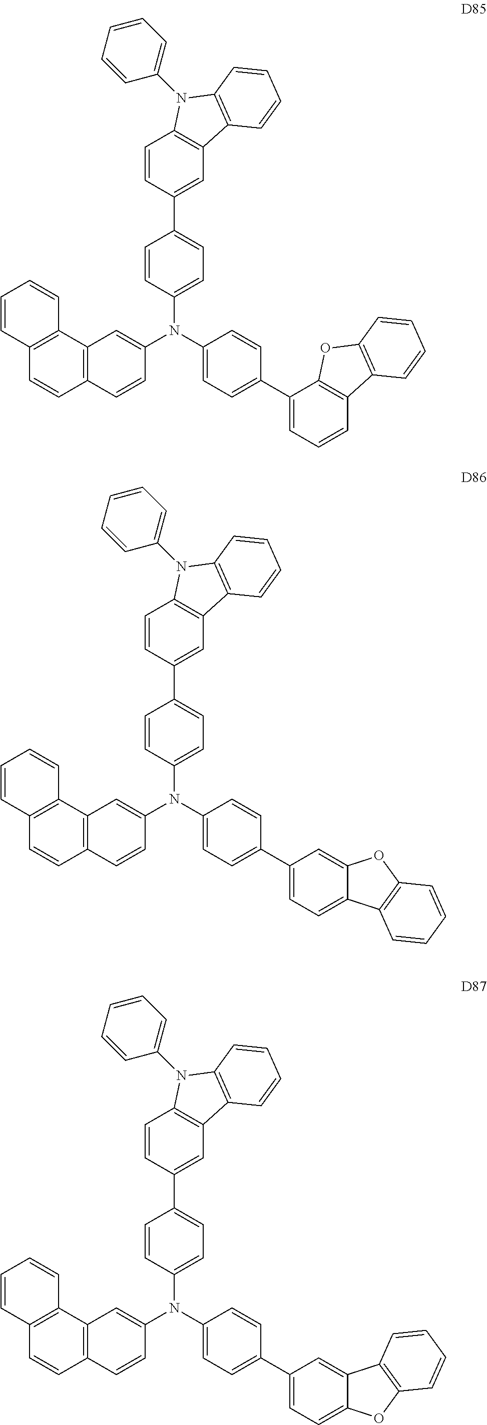 [SMILES: c1ccc(-n2c3ccccc3c3cc(-c4ccc(N(c5ccc(-c6cccc7c6oc6ccccc67)cc5)c5ccc6ccc7ccccc7c6c5)cc4)ccc32)cc1 ; c1ccc(-n2c3ccccc3c3cc(-c4ccc(N(c5ccc(-c6ccc7c(c6)oc6ccccc67)cc5)c5ccc6ccc7ccccc7c6c5)cc4)ccc32)cc1 ; c1ccc(-n2c3ccccc3c3cc(-c4ccc(N(c5ccc(-c6ccc7oc8ccccc8c7c6)cc5)c5ccc6ccc7ccccc7c6c5)cc4)ccc32)cc1]

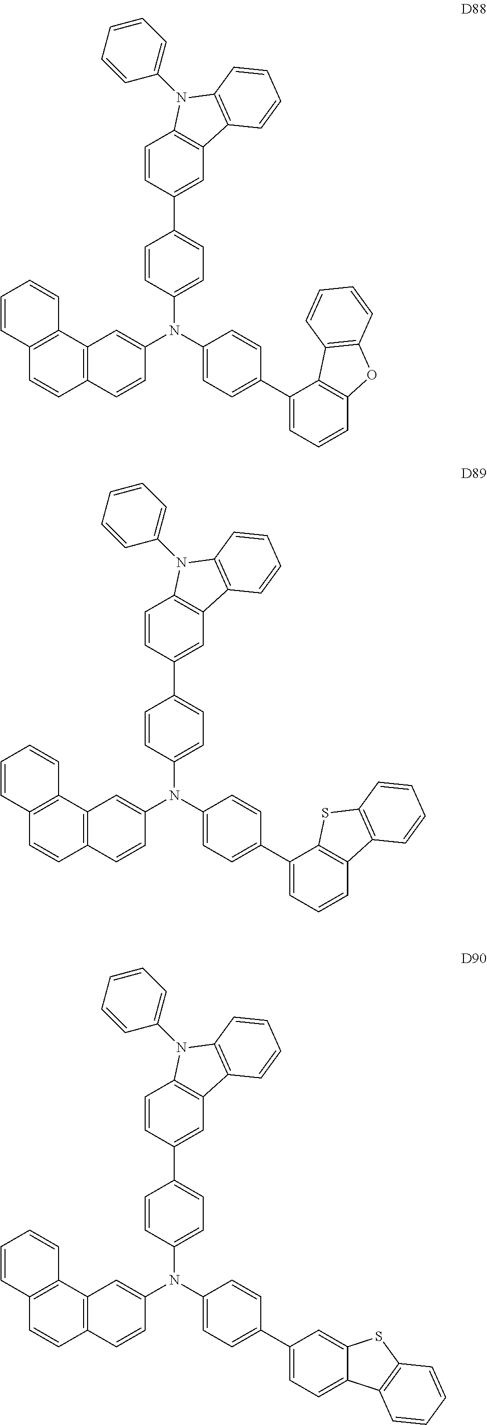 [SMILES: c1ccc(-n2c3ccccc3c3cc(-c4ccc(N(c5ccc(-c6cccc7oc8ccccc8c67)cc5)c5ccc6ccc7ccccc7c6c5)cc4)ccc32)cc1 ; c1ccc(-n2c3ccccc3c3cc(-c4ccc(N(c5ccc(-c6cccc7c6sc6ccccc67)cc5)c5ccc6ccc7ccccc7c6c5)cc4)ccc32)cc1 ; c1ccc(-n2c3ccccc3c3cc(-c4ccc(N(c5ccc(-c6ccc7c(c6)sc6ccccc67)cc5)c5ccc6ccc7ccccc7c6c5)cc4)ccc32)cc1]

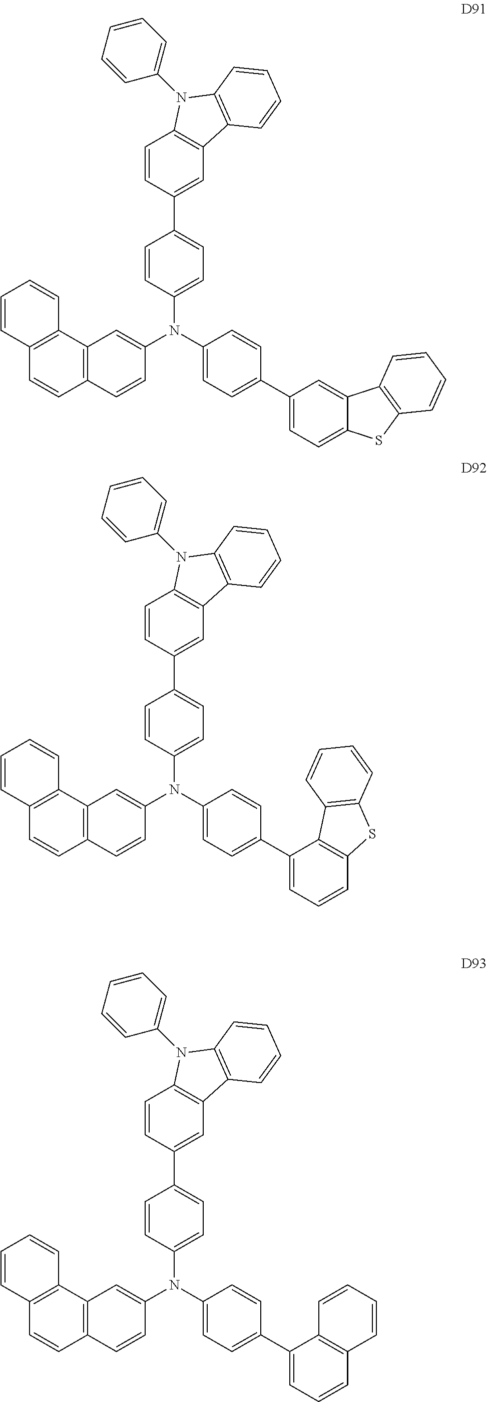 [SMILES: c1ccc(-n2c3ccccc3c3cc(-c4ccc(N(c5ccc(-c6ccc7sc8ccccc8c7c6)cc5)c5ccc6ccc7ccccc7c6c5)cc4)ccc32)cc1 ; c1ccc(-n2c3ccccc3c3cc(-c4ccc(N(c5ccc(-c6cccc7sc8ccccc8c67)cc5)c5ccc6ccc7ccccc7c6c5)cc4)ccc32)cc1 ; c1ccc(-n2c3ccccc3c3cc(-c4ccc(N(c5ccc(-c6cccc7ccccc67)cc5)c5ccc6ccc7ccccc7c6c5)cc4)ccc32)cc1]

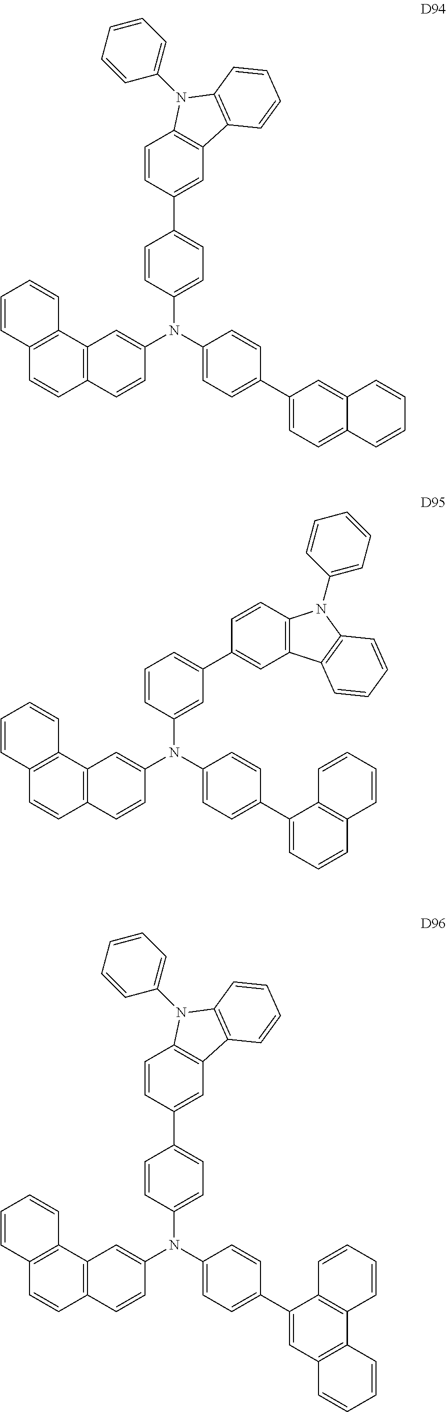 [SMILES: c1ccc(-n2c3ccccc3c3cc(-c4ccc(N(c5ccc(-c6ccc7ccccc7c6)cc5)c5ccc6ccc7ccccc7c6c5)cc4)ccc32)cc1 ; c1ccc(-n2c3ccccc3c3cc(-c4cccc(N(c5ccc(-c6cccc7ccccc67)cc5)c5ccc6ccc7ccccc7c6c5)c4)ccc32)cc1 ; c1ccc(-n2c3ccccc3c3cc(-c4ccc(N(c5ccc(-c6cc7ccccc7c7ccccc67)cc5)c5ccc6ccc7ccccc7c6c5)cc4)ccc32)cc1]

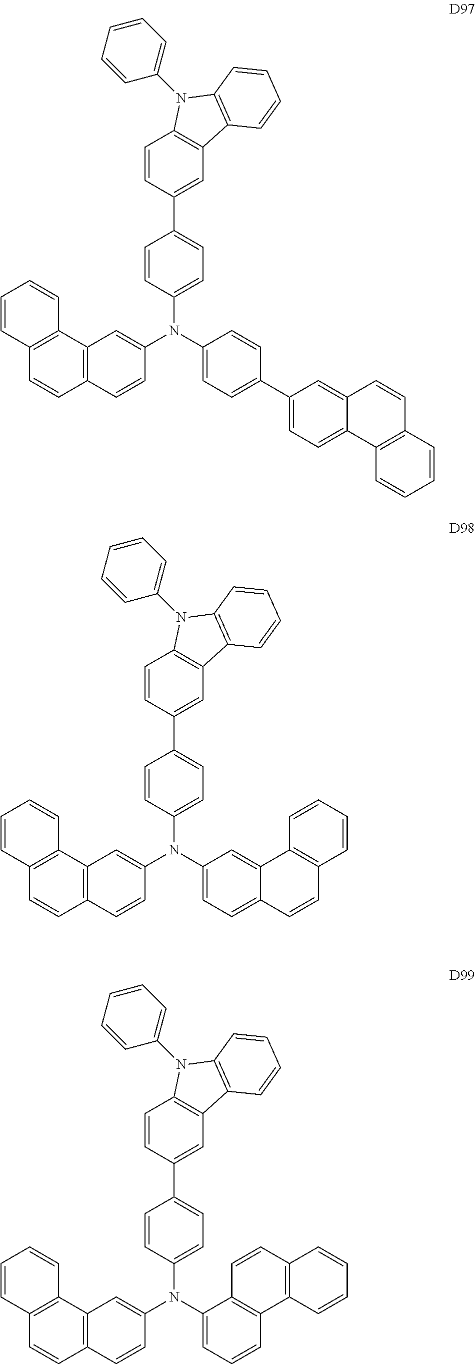 [SMILES: c1ccc(-n2c3ccccc3c3cc(-c4ccc(N(c5ccc(-c6ccc7c(ccc8ccccc87)c6)cc5)c5ccc6ccc7ccccc7c6c5)cc4)ccc32)cc1 ; c1ccc(-n2c3ccccc3c3cc(-c4ccc(N(c5ccc6ccc7ccccc7c6c5)c5ccc6ccc7ccccc7c6c5)cc4)ccc32)cc1 ; c1ccc(-n2c3ccccc3c3cc(-c4ccc(N(c5ccc6ccc7ccccc7c6c5)c5cccc6c5ccc5ccccc56)cc4)ccc32)cc1]

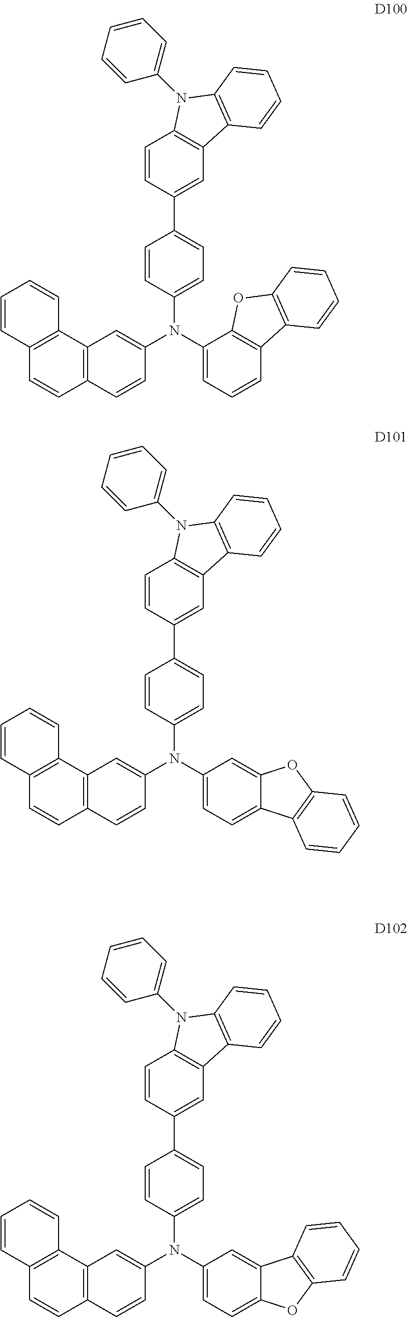 [SMILES: c1ccc(-n2c3ccccc3c3cc(-c4ccc(N(c5ccc6ccc7ccccc7c6c5)c5cccc6c5oc5ccccc56)cc4)ccc32)cc1 ; c1ccc(-n2c3ccccc3c3cc(-c4ccc(N(c5ccc6c(c5)oc5ccccc56)c5ccc6ccc7ccccc7c6c5)cc4)ccc32)cc1 ; c1ccc(-n2c3ccccc3c3cc(-c4ccc(N(c5ccc6ccc7ccccc7c6c5)c5ccc6oc7ccccc7c6c5)cc4)ccc32)cc1]

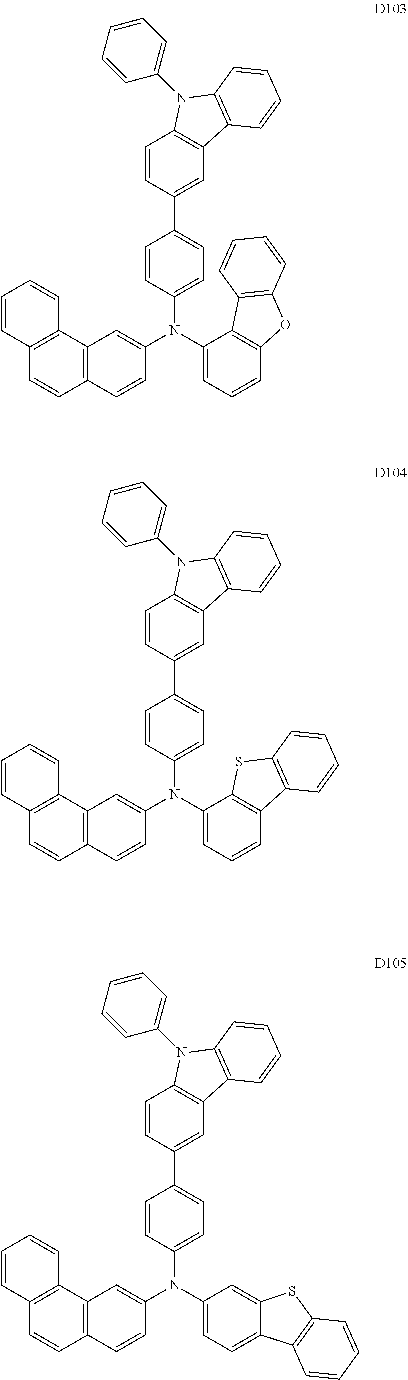 [SMILES: c1ccc(-n2c3ccccc3c3cc(-c4ccc(N(c5ccc6ccc7ccccc7c6c5)c5cccc6oc7ccccc7c56)cc4)ccc32)cc1 ; c1ccc(-n2c3ccccc3c3cc(-c4ccc(N(c5ccc6ccc7ccccc7c6c5)c5cccc6c5sc5ccccc56)cc4)ccc32)cc1 ; c1ccc(-n2c3ccccc3c3cc(-c4ccc(N(c5ccc6c(c5)sc5ccccc56)c5ccc6ccc7ccccc7c6c5)cc4)ccc32)cc1]

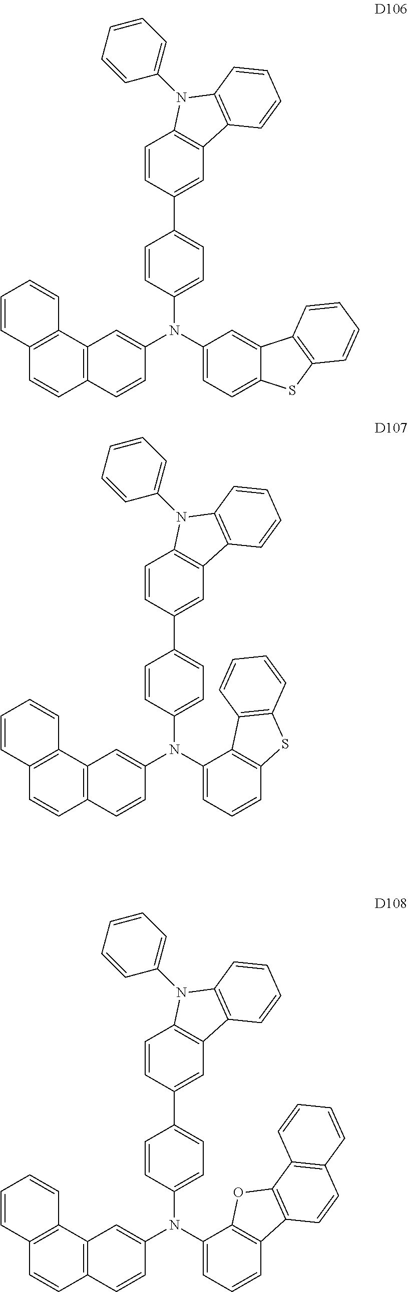 [SMILES: c1ccc(-n2c3ccccc3c3cc(-c4ccc(N(c5ccc6ccc7ccccc7c6c5)c5ccc6sc7ccccc7c6c5)cc4)ccc32)cc1 ; c1ccc(-n2c3ccccc3c3cc(-c4ccc(N(c5ccc6ccc7ccccc7c6c5)c5cccc6sc7ccccc7c56)cc4)ccc32)cc1 ; c1ccc(-n2c3ccccc3c3cc(-c4ccc(N(c5ccc6ccc7ccccc7c6c5)c5cccc6c5oc5c7ccccc7ccc65)cc4)ccc32)cc1]

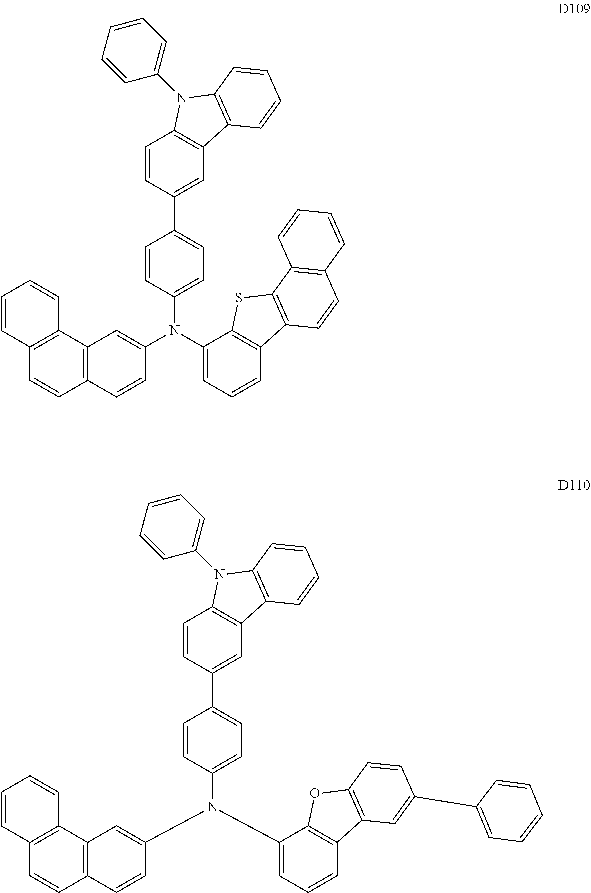 [SMILES: c1ccc(-n2c3ccccc3c3cc(-c4ccc(N(c5ccc6ccc7ccccc7c6c5)c5cccc6c5sc5c7ccccc7ccc65)cc4)ccc32)cc1 ; c1ccc(-c2ccc3oc4c(N(c5ccc(-c6ccc7c(c6)c6ccccc6n7-c6ccccc6)cc5)c5ccc6ccc7ccccc7c6c5)cccc4c3c2)cc1]

The amine compound represented by Formula 1 of an embodiment may include a phenanthrene moiety, and, for example, may have a feature in that the third position of the phenanthrene moiety is directly linked to the nitrogen atom of the amine. Thus, the amine compound of an embodiment may have improved hole transport property because the highest occupied molecular orbital (HOMO) level expands to thus contribute to the improvement in stability of the radical or radical cation state and the phenanthrene group having high planarity makes the intermolecular interaction more effective. In some embodiments, the amine compound of an embodiment has a phenanthrene skeleton at a position near to the center of the molecule, thereby suppressing or reducing excessive elevation of the deposition temperature and the deterioration of the material due to the deposition process. In some embodiments, the amine compound of an embodiment may have improved electron resistance and exciton resistance of the material by introducing two substituents among the substituents represented by Formula 2 to Formula 5 below into the amine moiety. Accordingly, the light emitting element of an embodiment including the amine compound of an embodiment may have an improvement in luminous efficiency and service life.

The hole transport region HTR in the light emitting element ED of an embodiment may further include a compound represented by Formula H-1 below:

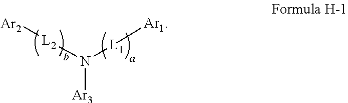

In Formula H-1 above, L₁ and L₂ may be each independently a direct linkage, a substituted or unsubstituted arylene group having 6 to 30 ring-forming carbon atoms, or a substituted or unsubstituted heteroarylene group having 2 to 30 ring-forming carbon atoms. a and b may be each independently an integer of 0 to 10. In some embodiments, when a or b is an integer of 2 or greater, a plurality of L₁'s and L₂'s may be each independently a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may be each independently a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms.

The compound represented by Formula H-1 above may be a monoamine compound. In some embodiments, the compound represented by Formula H-1 above may be a diamine compound in which at least one selected from among Ar₁ to Ar₃ includes the amine group as a substituent. In some embodiments, the compound represented by Formula H-1 above may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-1 may be represented by any one selected from among the compounds of Compound Group H below. However, the compounds listed in Compound Group H below are examples, and the compounds represented by Formula H-1 are not limited to those represented by Compound Group H below:

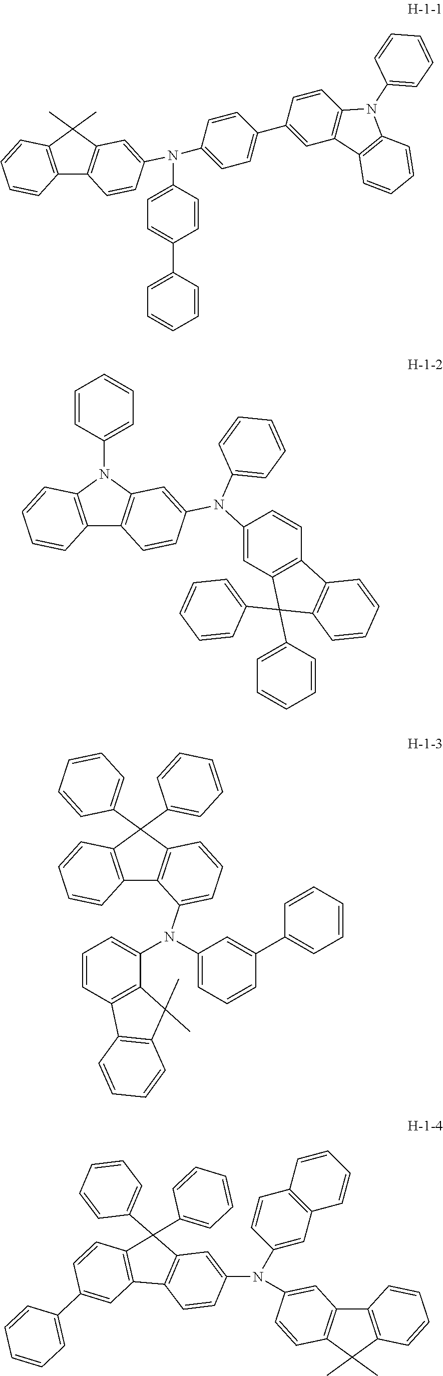 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

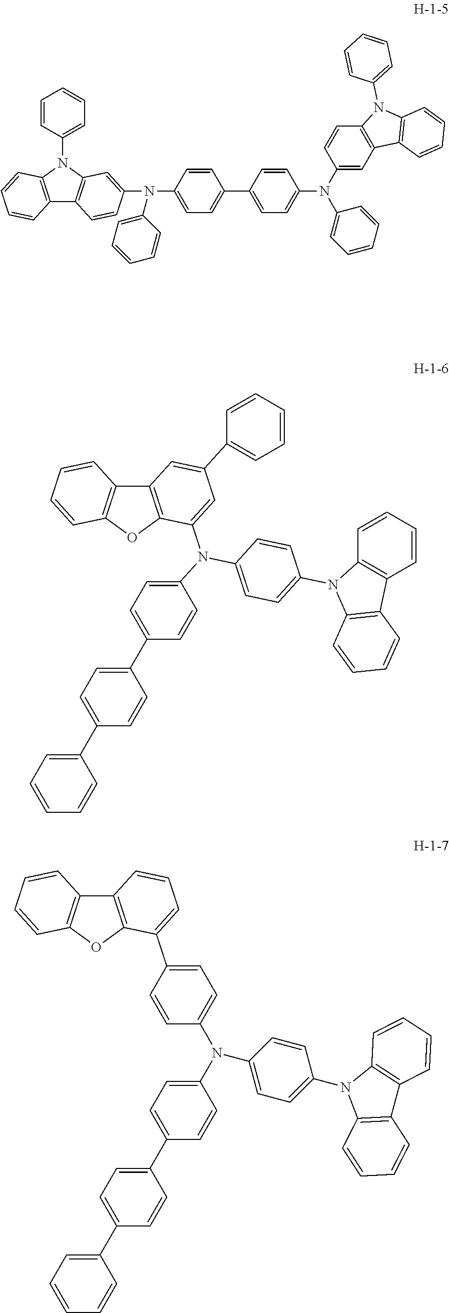 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

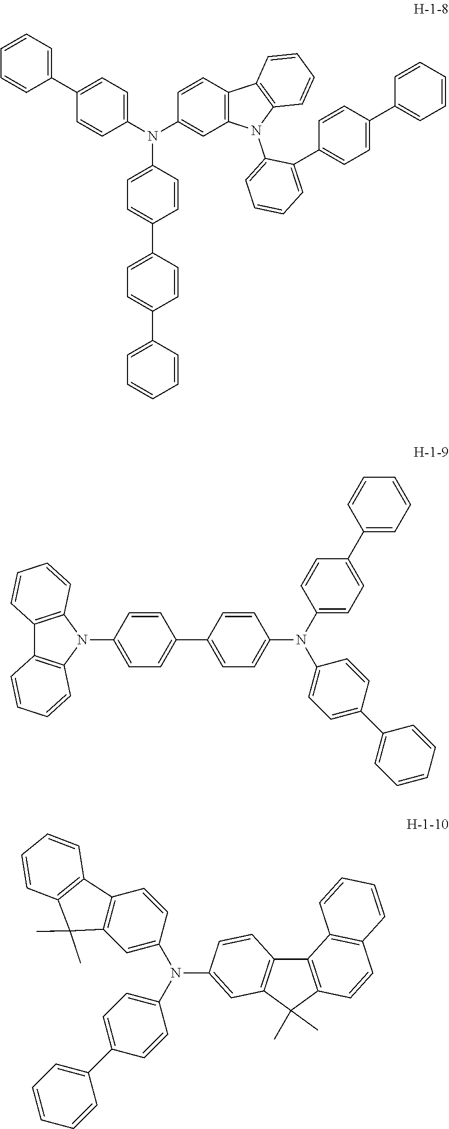 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

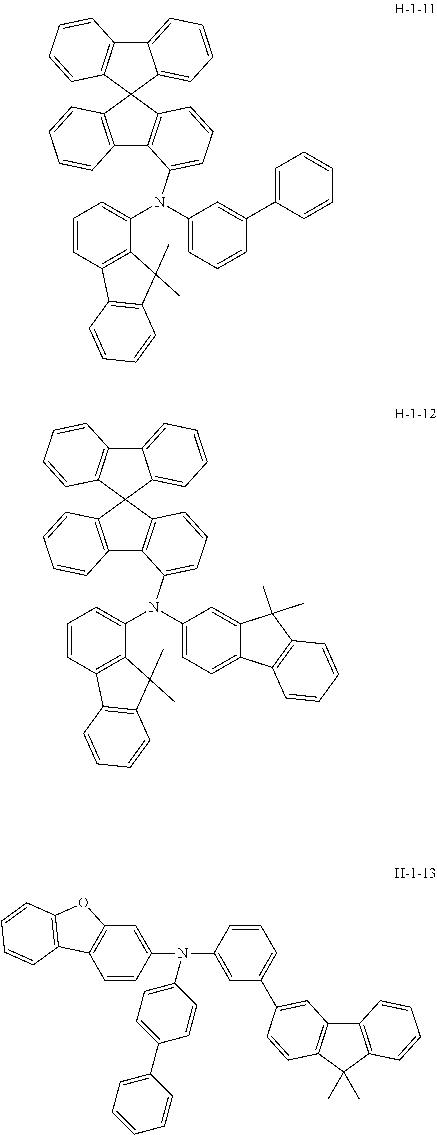 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

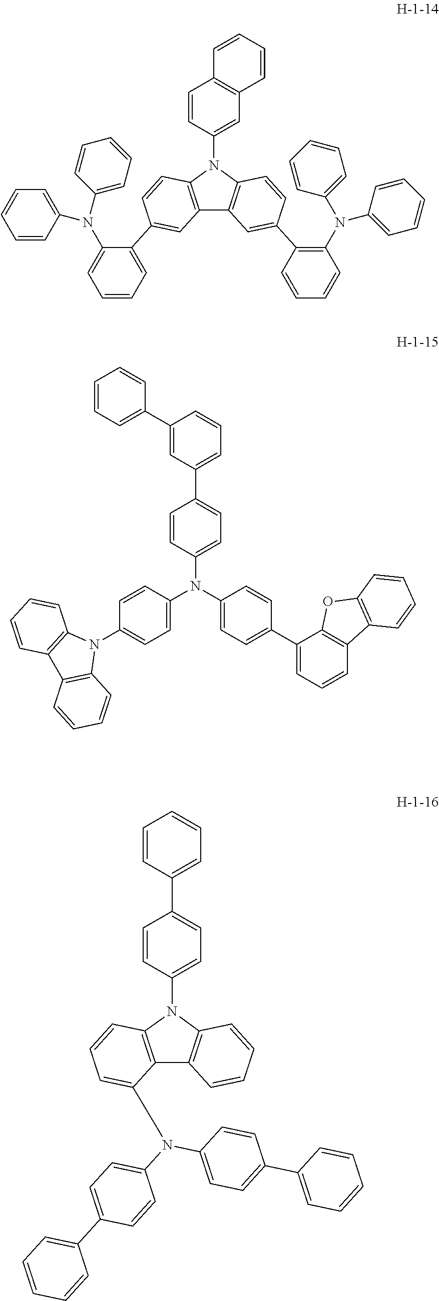 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

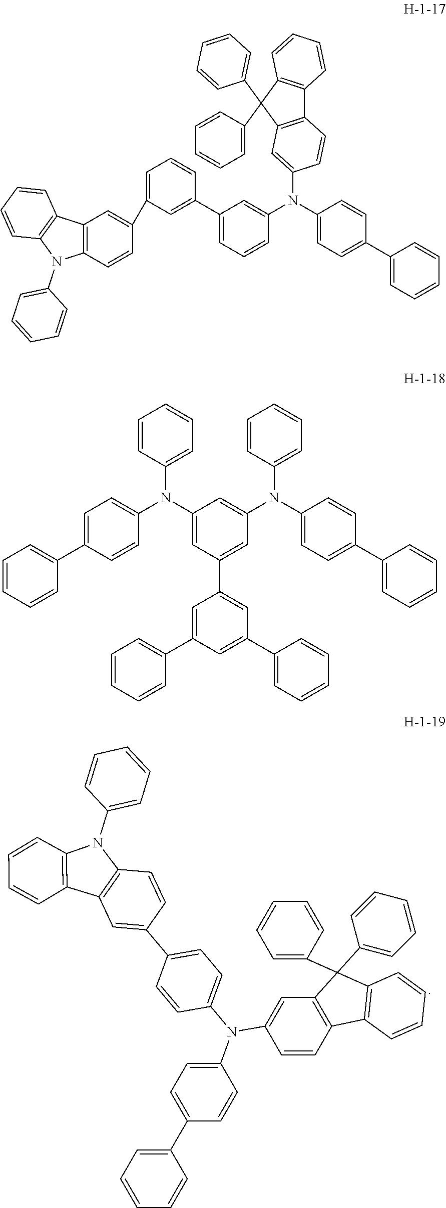 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine; N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′4″-tris(N,N-diphenylamino)triphenylamine (TDATA), 4,4′,4″-tris[N-(1-naphthyl)-N-phenylamino]-triphenylamine (1-TNATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

The hole transport region HTR may further include a carbazole-based derivative such as N-phenyl carbazole and/or polyvinyl carbazole, a fluorene-based derivative, a triphenyl amine-based derivative such as 4,4′,4″-tris(N-carbazolyl)triphenylamine (TCTA), and/or N,N′-bis(3-methylphenyl)-N,N′-diphenyl-[1,1′-biphenyl]-4,4′-diamine (TPD), N,N′-di(naphthalen-1-yl)-N,N′-diphenyl-benzidine (NPB), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 1,3-bis(N-carbazolyl)benzene (mCP), etc.

In some embodiments, the hole transport region HTR may further include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region in at least one of a hole injection layer HIL, a hole transport layer HTL, or an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 250 Å to about 1,000 Å. For example, when the hole transport region HTR includes the electron blocking layer EBL, the electron blocking layer EBL may have a thickness of about 10 Å to about 1,000 Å. If the thicknesses of the hole transport region HTR, the hole injection layer HIL, the hole transport layer HTL and the electron blocking layer EBL satisfy the above-described ranges, suitable or satisfactory hole transport properties may be achieved without a substantial increase in driving voltage.

The hole transport region HTR may further include a charge generating material to increase conductivity (e.g., electrical conductivity) in addition to the above-described materials. The charge generating material may be dispersed uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but the embodiment of the present disclosure is not limited thereto. For example, the p-dopant may include a metal halide compound such as CuI and/or RbI, a quinone derivative such as tetracyanoquinodimethane (TCNQ) and/or 2,3,5,6-tetrafluoro-7,7,8,8-tetracyanoquinodimethane (F4-TCNQ), a metal oxide such as tungsten oxide and/or molybdenum oxide, a cyano group-containing compound such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but embodiments of the present disclosure are not limited thereto.

As described above, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be contained in the hole transport region HTR may be used as a material to be contained in the buffer layer. The electron blocking layer EBL is a layer that prevents or reduces the injection of electrons from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

The emission layer EML in the light emitting element ED of an embodiment may emit blue light. The light emitting element ED of an embodiment may include the above-described amine compound of an embodiment in the hole transport region HTR, thereby exhibiting high efficiency and long service life characteristics in the blue light emitting region. However, embodiments of the present disclosure are not limited thereto.

In the light emitting element ED of an embodiment, the emission layer EML may include an anthracene derivative, a pyrene derivative, a fluoranthene derivative, a chrysene derivative, a dehydrobenzanthracene derivative, and/or a triphenylene derivative. For example, the emission layer EML may include the anthracene derivative and/or the pyrene derivative.

In each of the light emitting elements ED of embodiments illustrated in FIGS. 3 to 6, the emission layer EML may include a host and a dopant, and the emission layer EML may include a compound represented by Formula E-1 below. The compound represented by Formula E-1 below may be used as a fluorescent host material.

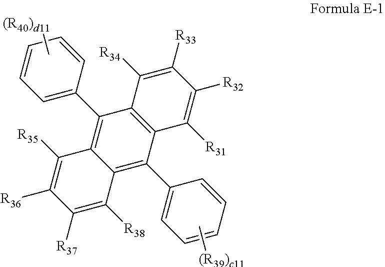

In Formula E-1, R₃₁ to R₄₀ may be each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In some embodiments, R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may be each independently an integer of 0 to 5.

Formula E-1 may be represented by any one selected from among Compound E1 to Compound E19 below:

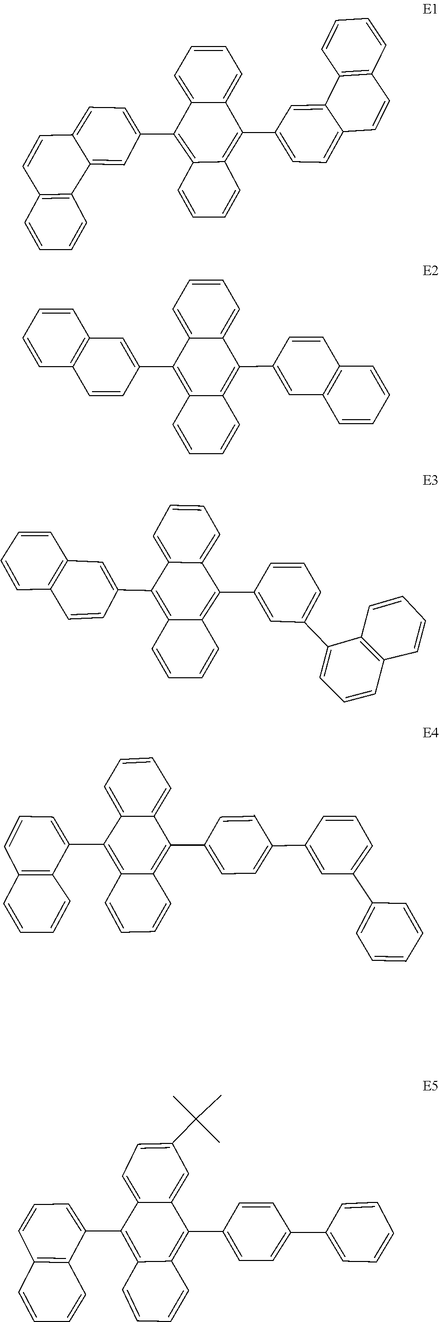

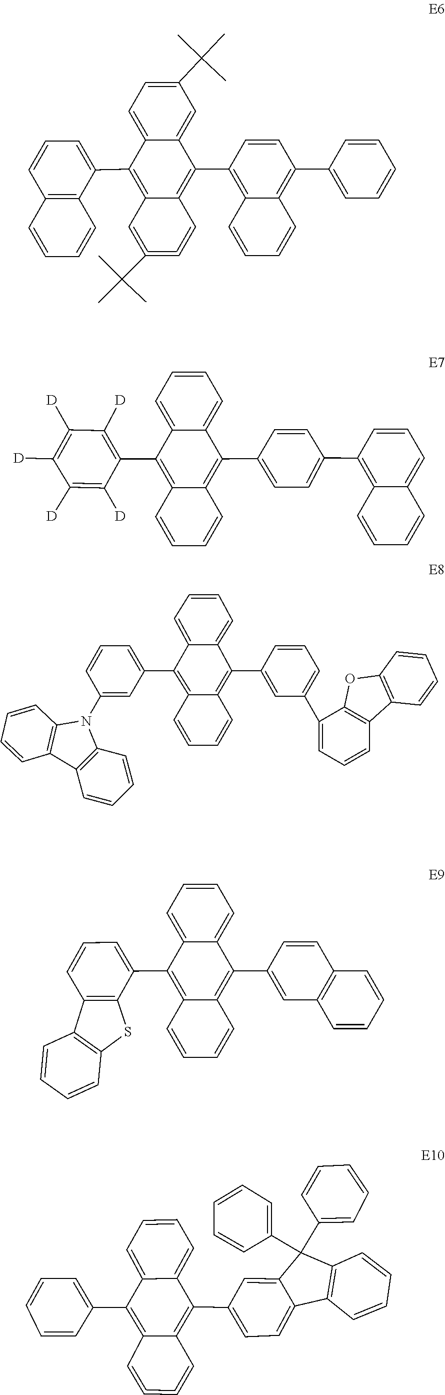

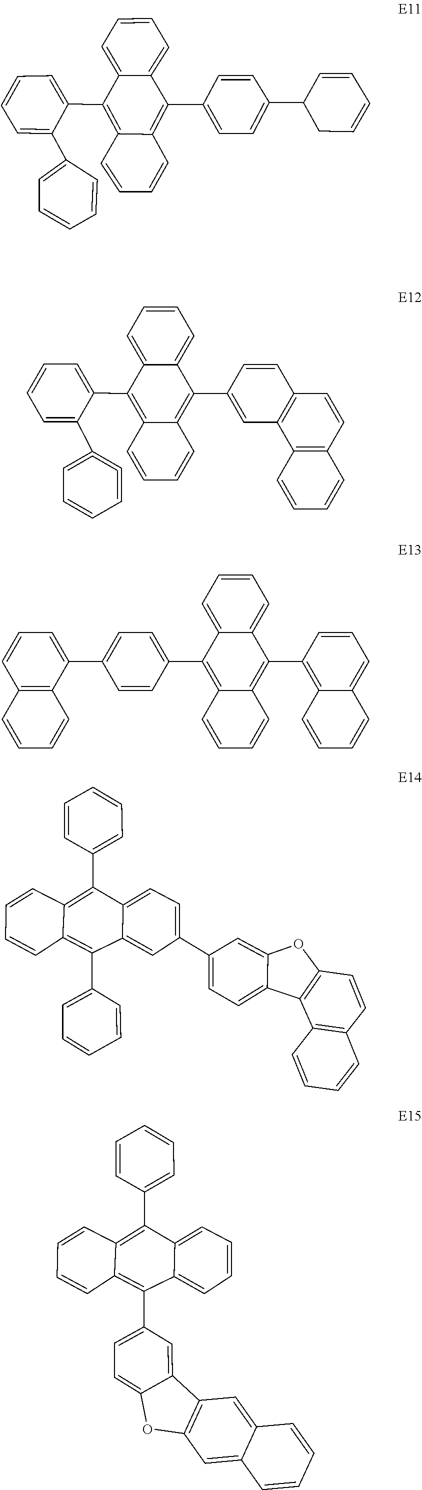

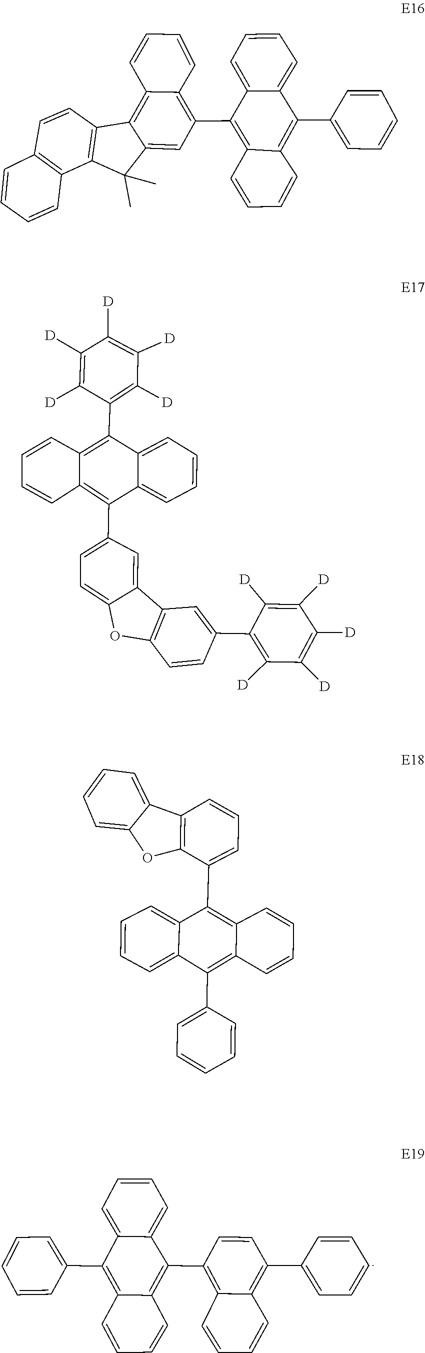

In an embodiment, the emission layer EML may include a compound represented by Formula E-2a or Formula E-2b below. The compound represented by Formula E-2a or Formula E-2b below may be used as a phosphorescent host material.

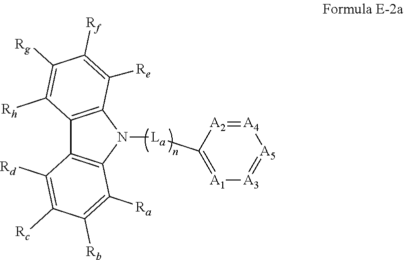

In Formula E-2a, and a may be an integer of 0 to 10, La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or more, a plurality of La's may be each independently a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may be each independently N or CRᵢ. Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Rₐ to Rᵢ may be bonded to an adjacent group to form a hydrocarbon ring or a heterocycle containing N, O, S, etc. as a ring-forming atom.

In Formula E-2a, two or three selected from among A₁ to A₅ may be N, and the rest may be CRᵢ.

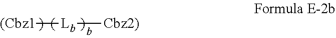

In Formula E-2b, Cbz1 and Cbz2 may be each independently an unsubstituted carbazole group, or a carbazole group substituted with an aryl group having 6 to 30 ring-forming carbon atoms. L_{b} is a direct linkage, a substituted or unsubstituted arylene group having 6 to 30 ring-forming carbon atoms, or a substituted or unsubstituted heteroarylene group having 2 to 30 ring-forming carbon atoms. In some embodiments, b is an integer of 0 to 10, and when b is an integer of 2 or more, a plurality of L_{b}'s may be each independently a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among the compounds of Compound Group E-2 below. However, the compounds listed in Compound Group E-2 below are examples, and the compound represented by Formula E-2a or Formula E-2b is not limited to those represented in Compound Group E-2 below.

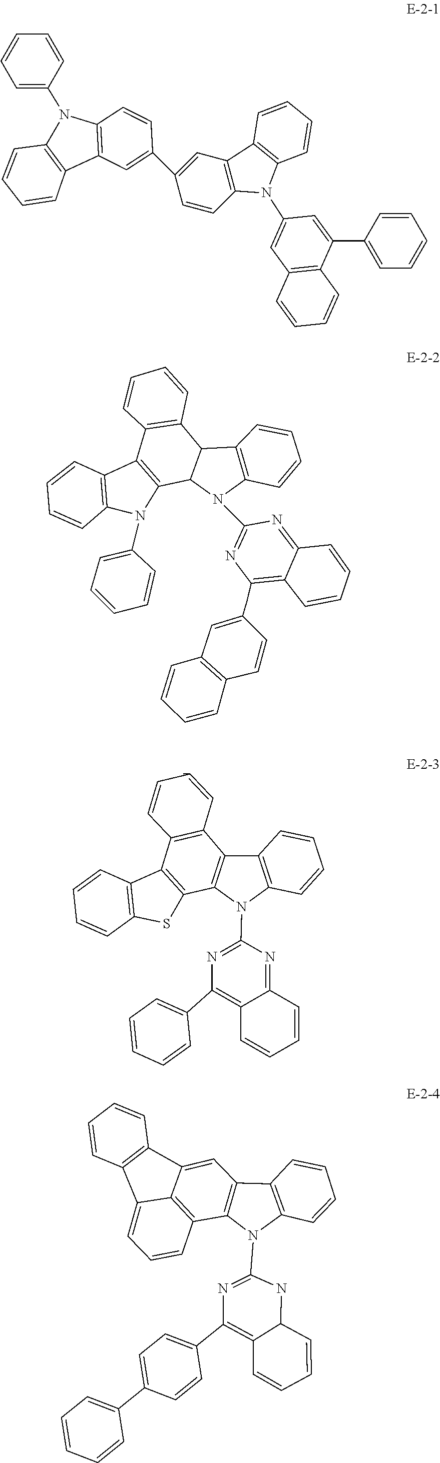 [SMILES: C1=CCc2c(n(-c3ccccc3)c3ccc(-c4ccc5c(c4)c4ccccc4n5-c4cc(-c5ccccc5)c5ccccc5c4)cc23)C=C1 ; c1ccc(-n2c3c(c4ccccc42)-c2ccccc2C2c4ccccc4N(c4nc(Cc5ccc6ccccc6c5)c5ccccc5n4)C32)cc1 ; Cc1ccc2c(c1)c1c3ccccc3n(-c3nc(-c4ccccc4)c4ccccc4n3)c1c1sc3ccccc3c21 ; C1=CC2=C(c3ccc(-c4ccccc4)cc3)N=C(n3c4ccccc4c4cc5c6c(cccc6c43)-c3ccccc3-5)NC2C=C1]

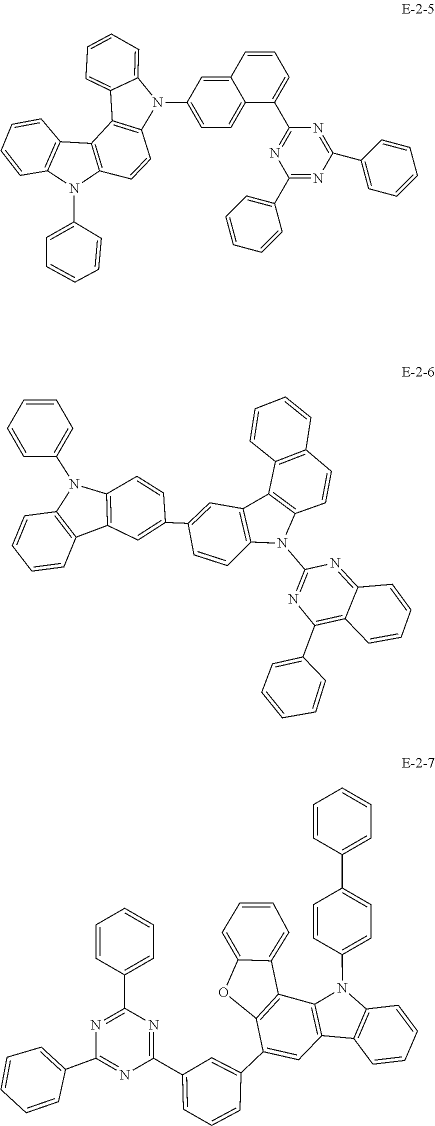 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1]

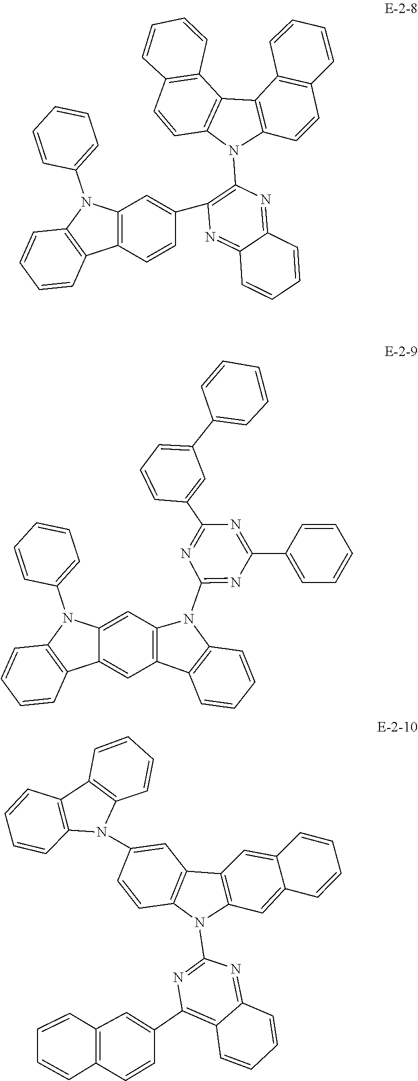 [SMILES: c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1]

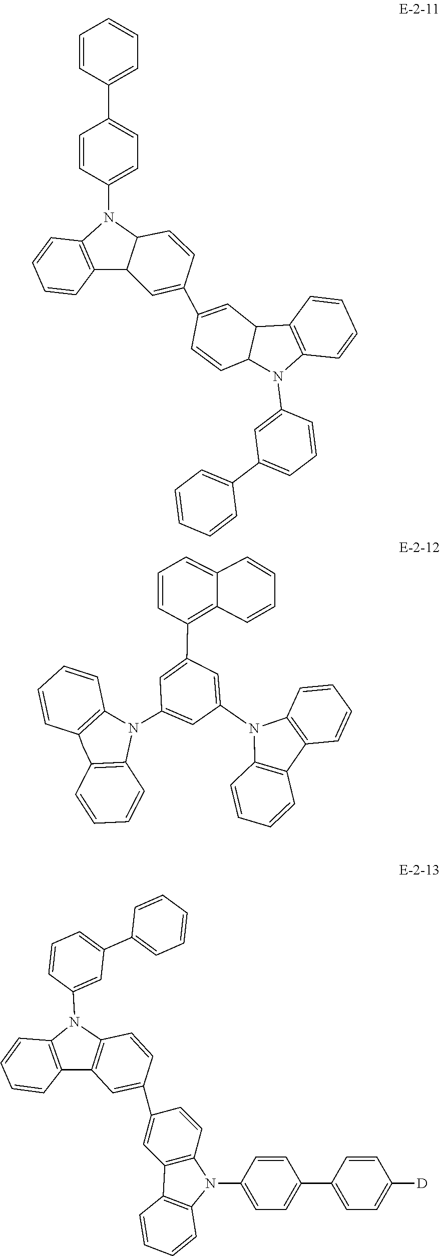

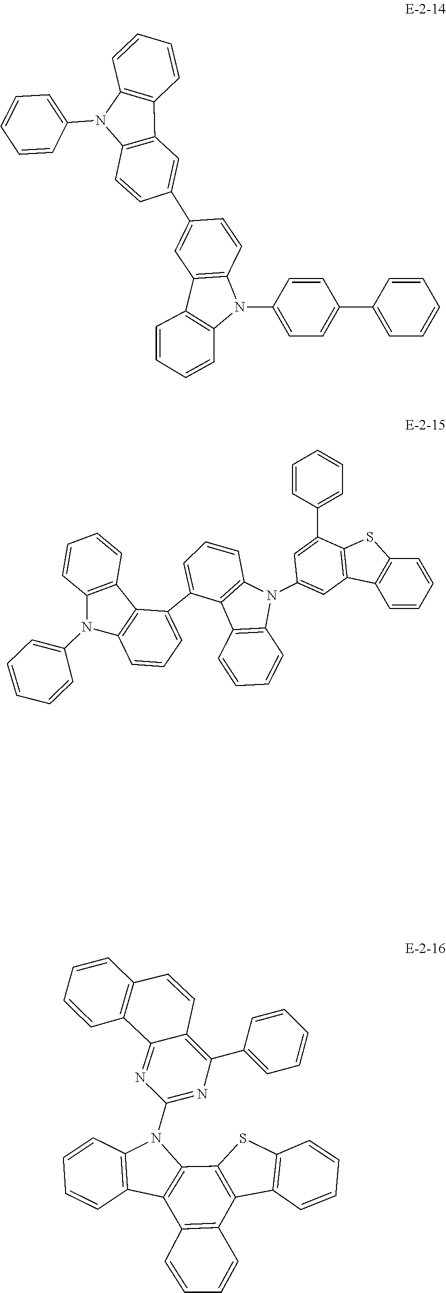 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2nc(-n3c4ccccc4c4c5ccccc5c5c6ccccc6sc5c43)nc3c2ccc2ccccc23)cc1]

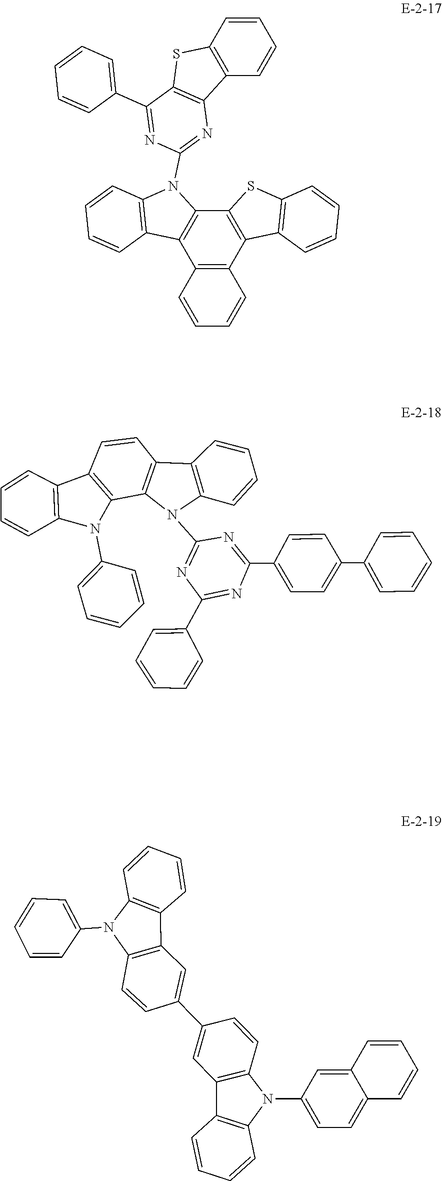 [SMILES: c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

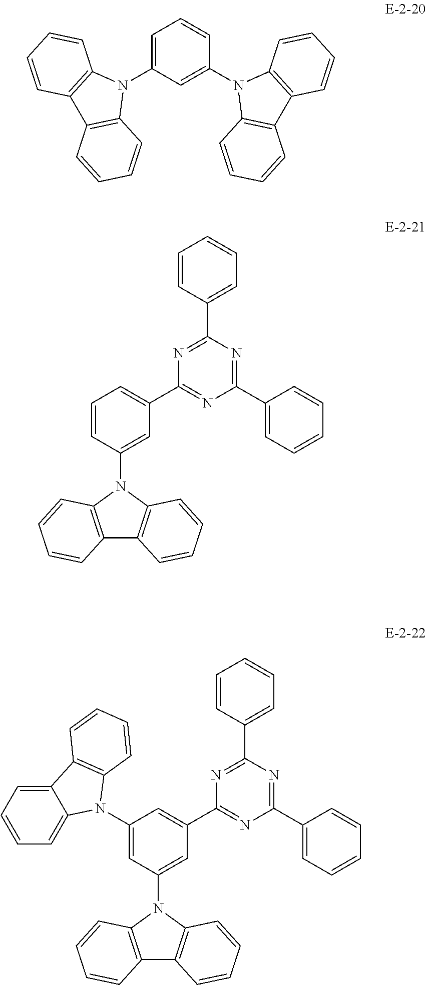 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

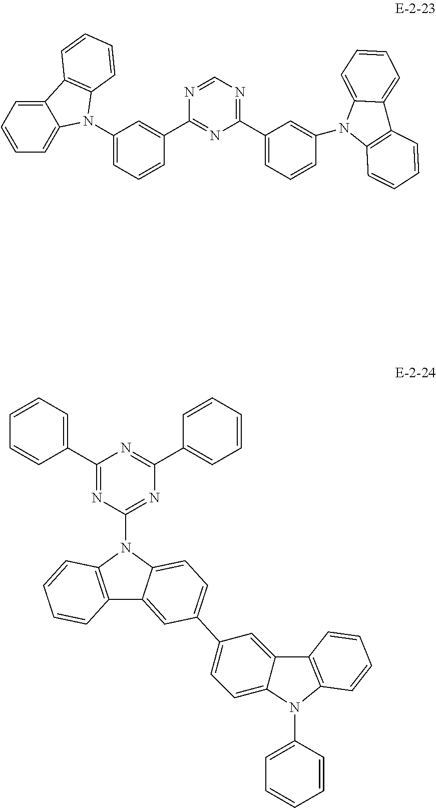 [SMILES: c1cc(-c2ncnc(-c3cccc(-n4c5ccccc5c5ccccc54)c3)n2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include any suitable material generally used in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, embodiments of the present disclosure are not limited thereto, for example, tris(8-hydroxyquinolinato)aluminum (Alq₃), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO3), octaphenylcyclotetra siloxane (DPSiO4), etc. may be used as a host material.

The emission layer EML may include a compound represented by Formula M-a or Formula M-b below. The compound represented by Formula M-a or Formula M-b below may be used as a phosphorescent dopant material. In some embodiments, the compound represented by Formula M-a or Formula M-b in an embodiment may be used as an auxiliary dopant material.

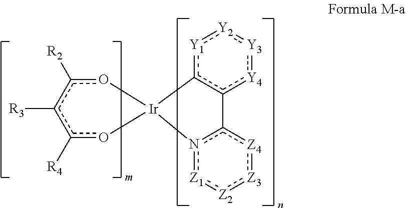

In Formula M-a above, Y₁ to Y₄ and Z₁ to Z₄ may be each independently CR₁ or N, R₁ to R₄ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m is 0 or 1, and n is 2 or 3. In Formula M-a, when m is 0, n is 3, and when m is 1, n is 2.

The compound represented by Formula M-a may be represented by any one selected from among Compound M-a1 to Compound M-a25 below. However, Compounds M-a1 to M-a25 below are examples, and the compound represented by Formula M-a is not limited to those represented by Compounds M-a1 to M-a25 below.

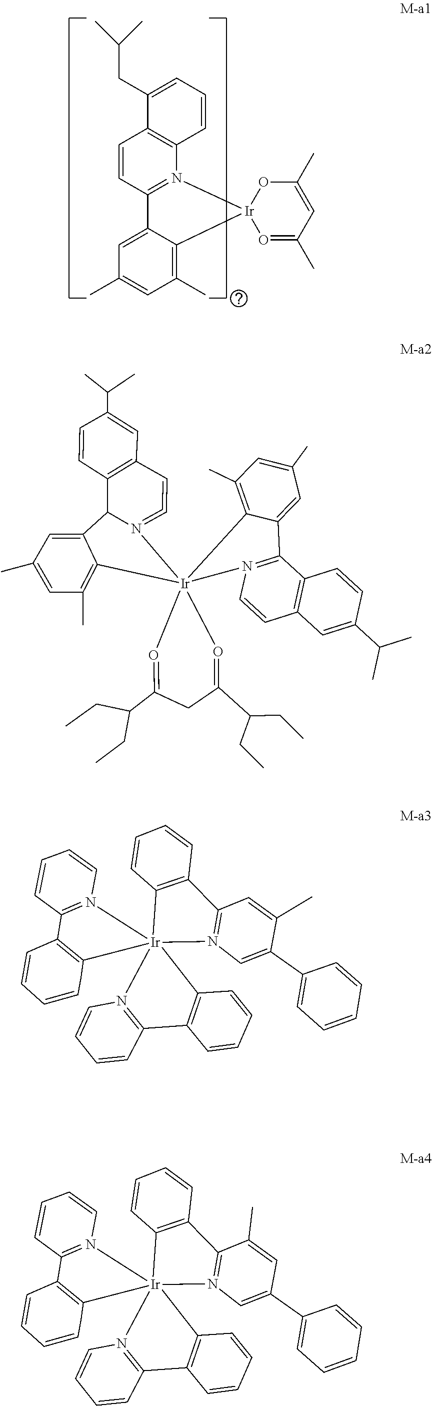

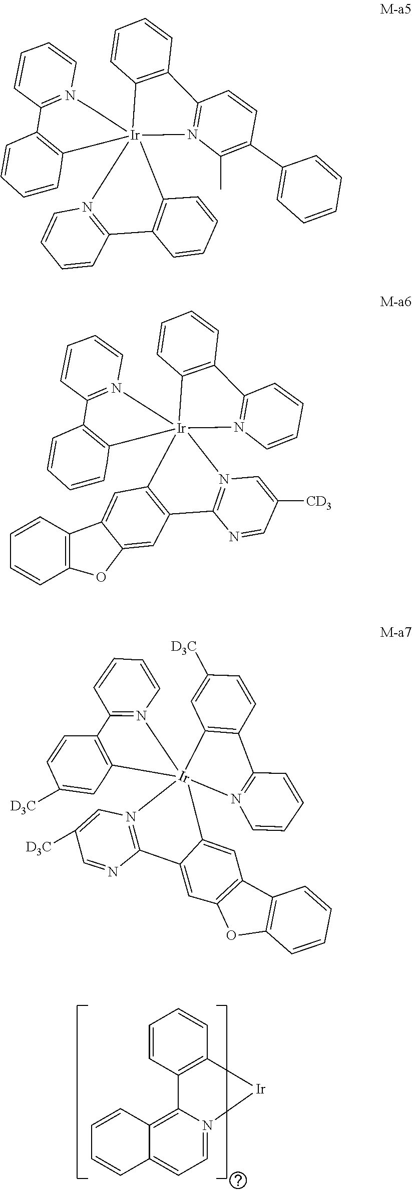

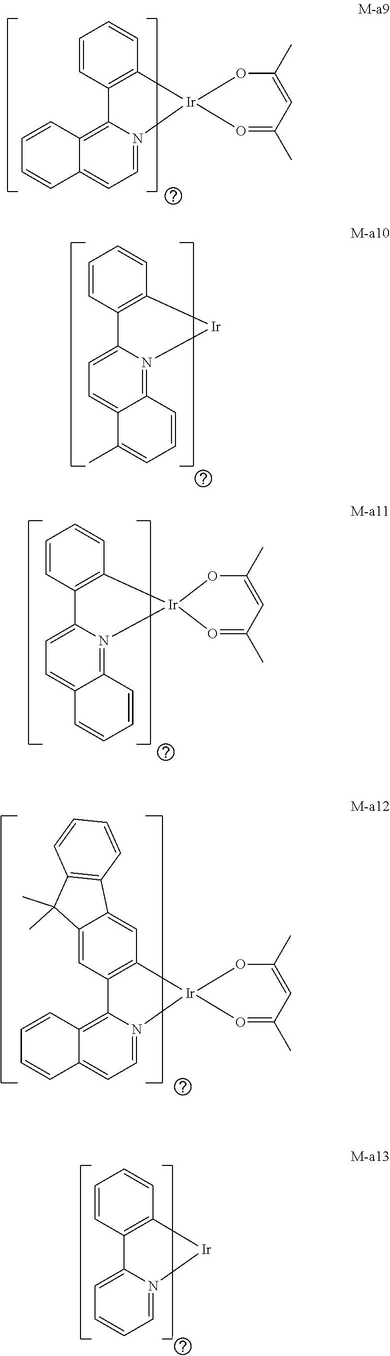

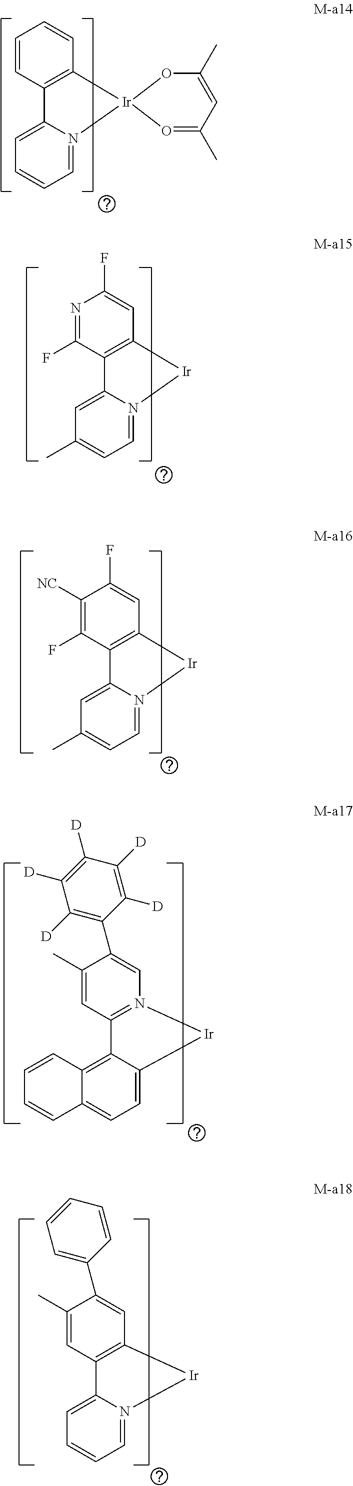

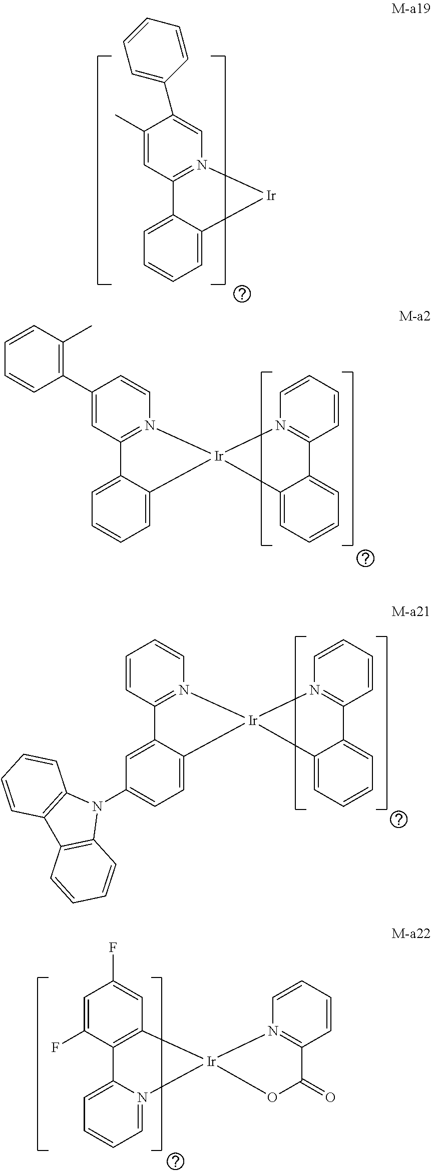

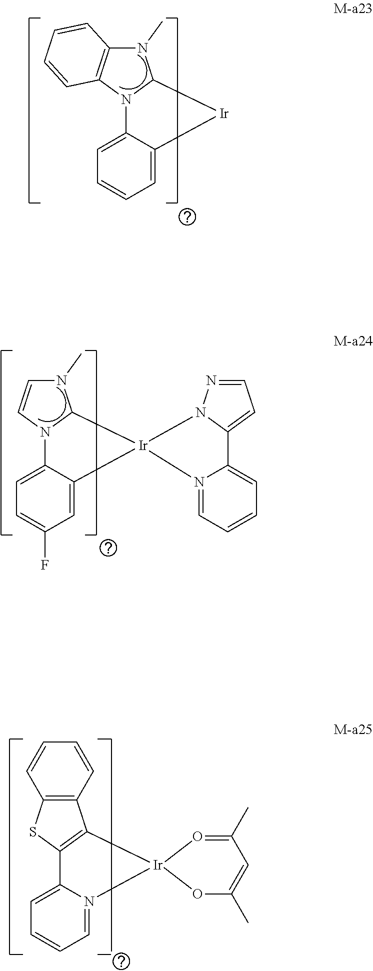

Compound M-a1 and Compound M-a2 may be used as a red dopant material, and Compound M-a3 to Compound M-a7 may be used as a green dopant material.

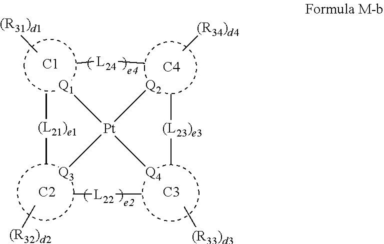

In Formula M-b, Q₁ to Q₄ are each independently C or N, and C1 to C4 are each independently a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ are each independently a direct linkage,

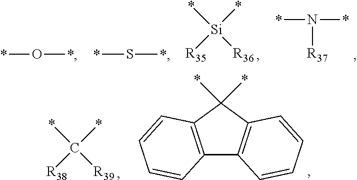

substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 are each independently 0 or 1. R₃₁ to R₃₉ are each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and d1 to d4 are each independently an integer of 0 to 4.

The compound represented by Formula M-b may be used as a blue phosphorescent dopant or a green phosphorescent dopant. In some embodiments, the compound represented by Formula M-b may be further included as an auxiliary dopant in the emission layer EML in an embodiment.

The compound represented by Formula M-b may be represented by any one selected from among the compounds below. However, the compounds below are examples, and the compound represented by Formula M-b is not limited to those represented by the compounds below.

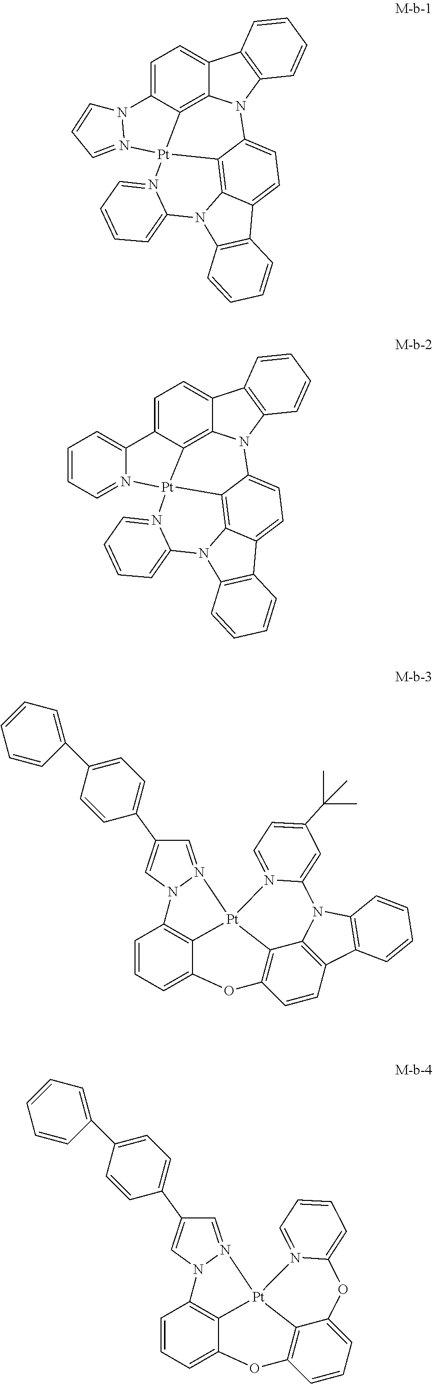

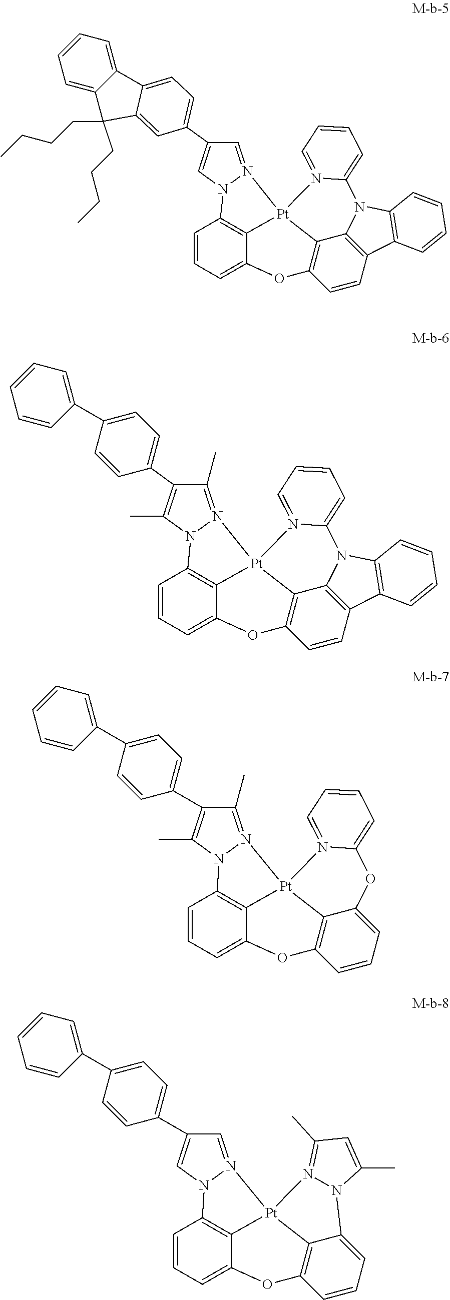

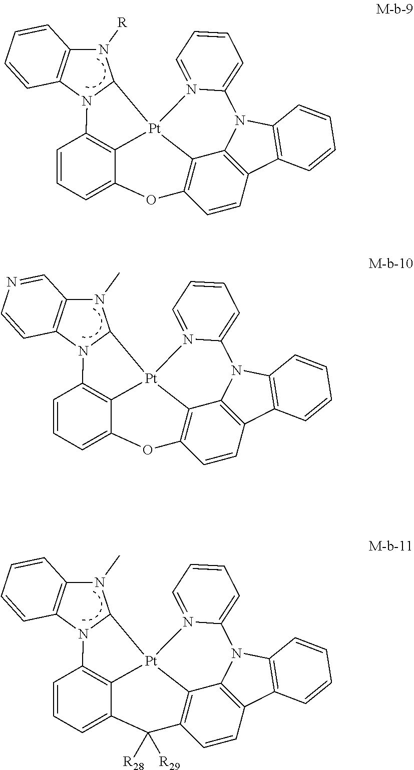

In the compounds above, R, R₃₈, and R₃₉ may be each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may further include a compound represented by any one selected from among Formula F-a to Formula F-c below. The compound represented by Formula F-a to Formula F-c below may be used as a fluorescence dopant material.

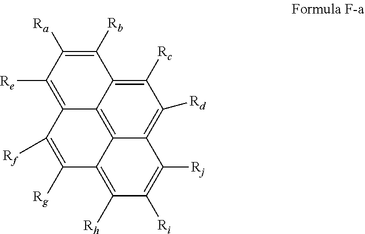

In Formula F-a above, two selected from among Rₐ to Rⱼ may each independently be substituted with

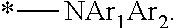

The others, which are not substituted with

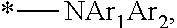

among Rₐ to Rⱼ may be each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In

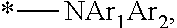

Ar₁ and Ar₂ may be each independently a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

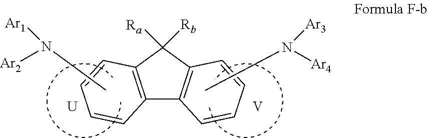

In Formula F-b above, Rₐ and R_{b} may be each independently a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Ar₁ to Ar₄ may be each independently a substituted or unsubstituted aryl group having 6 to 30 ring-forming carbon atoms, or a substituted or unsubstituted heteroaryl group having 2 to 30 ring-forming carbon atoms.

In Formula F-b, U and V may be each independently a substituted or unsubstituted hydrocarbon ring having 5 to 30 ring-forming carbon atoms, or a substituted or unsubstituted heterocycle having 2 to 30 ring-forming carbon atoms.

In Formula F-b, the number of rings represented by U and V may be each independently 0 or 1. For example, in Formula F-b, it means that when the number of U or V is 1, one ring constitutes a fused ring at a portion indicated by U or V, and when the number of U or V is 0, a ring indicated by U or V does not exist. For example, when the number of U is 0 and the number of V is 1, or when the number of U is 1 and the number of V is 0, the fused ring having a fluorene core in Formula F-b may be a cyclic compound having four rings. In some embodiments, when each number of U and V is 0, the fused ring in Formula F-b may be a cyclic compound having three rings. In some embodiments, when each number of U and V is 1, the fused ring having a fluorene core in Formula F-b may be a cyclic compound having five rings.

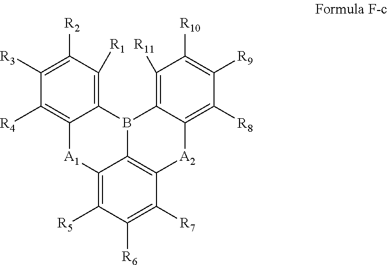

In Formula F-c, A₁ and A₂ may be each independently O, S, Se, or NRₘ, and Rm may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ are each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring.

In Formula F-c, A₁ and A₂ may each independently be bonded to substituents of an adjacent ring to form a condensed ring. For example, when A₁ and A₂ are each independently NRₘ, A₁ may be bonded to R₄ or R₅ to form a ring. In some embodiments, A₂ may be bonded to R₇ or R₈ to form a ring.

In an embodiment, the emission layer EML may include, any suitable dopant material generally used in the art, styryl derivatives (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), and/or N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and/or derivatives thereof (e.g., 2,5,8,11-tetra-t-butylperylene (TBP)), pyrene and/or derivatives thereof (e.g., 1,1′-dipyrene, 1,4-dipyrenylbenzene, 1,4-bis(N,N-diphenylamino)pyrene), etc.

In an embodiment, when a plurality of emission layers EML are included, at least one emission layer EML may include any suitable phosphorescence dopant material generally used in the art. For example, a metal complex containing iridium (Ir), platinum (Pt), osmium (Os), aurum (Au), titanium (Ti), zirconium (Zr), hafnium (Hf), europium (Eu), terbium (Tb), or thulium (Tm) may be used as a phosphorescent dopant. For example, iridium(III) bis(4,6-difluorophenylpyridinato-N,C2′) picolinate (Firpic), bis(2,4-difluorophenylpyridinato)-tetrakis(1-pyrazolyl)borate iridium(III) (Fir6), and/or platinum octaethyl porphyrin (PtOEP) may be used as a phosphorescent dopant. However, embodiments of the present disclosure are not limited thereto.

In some embodiments, the emission layer EML may include a hole transport host and an electron transport host. The emission layer EML may include an auxiliary dopant and a light emitting dopant. In some embodiments, a phosphorescent dopant material and/or a thermally delayed fluorescent dopant material may be included as the auxiliary dopant. For example, the emission layer EML in an embodiment may include the hole transport host, the electron transport host, the auxiliary dopant, and the light emitting dopant.

In some embodiments, in the emission layer EML, an exciplex may be formed by the hole transport host and the electron transport host. In this case, a triplet energy of the exciplex formed by the hole transport host and the electron transport host may correspond to T1 that is a gap between a lowest unoccupied molecular orbital (LUMO) energy level of the electron transport host and a HOMO energy level of the hole transport host.

In an embodiment, the triplet energy (T1) of the exciplex formed by the hole transport host and the electron transport host may be about 2.4 eV to about 3.0 eV. In some embodiments, the triplet energy of the exciplex may be a value smaller than an energy gap of each host material. Therefore, the exciplex may have a triplet energy of about 3.0 eV or less that is an energy gap between the hole transport host and the electron transport host.

In some embodiments, at least one emission layer EML may include a quantum dot material. The core of the quantum dot may be selected from a Group II-VI compound, a Group III-VI compound, a Group 1-III-IV compound, a Group III-V compound, a Group III-II-V compound, a Group IV-VI compound, a Group IV element, a Group IV compound, or a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and a mixture thereof.

The Group III-VI compound may include a binary compound such as In₂S₃ and/or In₂Se₃, a ternary compound such as InGaS₃ and/or InGaSe₃, or any combination thereof.

The Group I-III-VI compound may be selected from a ternary compound selected from the group consisting of AgInS, AgInS₂, CuInS, CuInS₂, AgGaS₂, CuGaS₂, CuGaO₂, AgGaO₂, AgAlO₂, and a mixture thereof, and/or a quaternary compound such as AgInGaS₂ and/or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GalnNSb, GaInPAs, GalnPSb, InAlNP, InAlNAs, InAlNSb, InAlPAs, InAlPSb, and a mixture thereof. In some embodiments, the Group III-V compound may further include a Group II metal. For example, InZnP, etc. may be selected as a Group III-II-V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

In this case, a binary compound, a ternary compound, or a quaternary compound may be present in a particle with a uniform (e.g., substantially uniform) concentration distribution, or may be present in the same particle with a partially different concentration distribution. In some embodiments, a core/shell structure in which one quantum dot surrounds another quantum dot may also be possible. The core/shell structure may have a concentration gradient in which the concentration of elements present in the shell decreases toward the core.

In some embodiments, the quantum dot may have the above-described core/shell structure including a core containing nanocrystals and a shell surrounding the core. The shell of the quantum dot may serve as a protection layer to prevent or reduce the chemical deformation of the core so as to maintain semiconductor properties, and/or a charging layer to impart electrophoresis properties to the quantum dot. The shell may be a single layer or a multilayer. An example of the shell of the quantum dot may include a metal and/or non-metal oxide, a semiconductor compound, or a combination thereof.

For example, the metal and/or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO, and/or a ternary compound such as MgAl₂O₄, CoFe₂O₄, NiFe₂O₄, and/or CoMn₂O₄, but embodiments of the present disclosure are not limited thereto.

In some embodiments, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments of the present disclosure are not limited thereto.

The quantum dot may have a full width of half maximum (FWHM) of a light emitting wavelength spectrum of about 45 nm or less, about 40 nm or less, and, for example, about 30 nm or less, and color purity and/or color reproducibility may be improved in the above range. In some embodiments, light emitted through such a quantum dot is emitted in all directions (e.g., substantially all directions), and thus, a wide viewing angle may be improved.

The form of a quantum dot is not particularly limited as long as it is a form generally used in the art. For example, a quantum dot in the form of spherical, pyramidal, multi-arm, or cubic nanoparticles, nanotubes, nanowires, nanofibers, nanoparticles, etc. may be used.

The quantum dot may control the color of emitted light according to the particle size thereof. Accordingly, the quantum dot may have various suitable light emission colors such as blue, red, and green.

In each of the light emitting elements ED of embodiments illustrated in FIGS. 3 to 6, the electron transport region ETR is provided on the emission layer EML. The electron transport region ETR may include at least one of the hole blocking layer HBL, the electron transport layer ETL, or the electron injection layer EIL, but embodiments of the present disclosure are not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL, an electron transport layer ETL/buffer layer/electron injection layer EIL are stacked in order from the emission layer EML, but embodiments of the present disclosure are not limited thereto. The electron transport region ETR may have a thickness, for example, from about 1,000 Å to about 1,500 Å.

The electron transport region ETR may be formed by using various suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1 below:

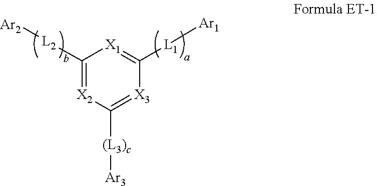

In Formula ET-1, at least one selected from among X₁ to X₃ is N, and the rest are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may be each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may be each independently an integer of 0 to 10. In Formula ET-1, L₁ to L₃ may be each independently a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c are an integer of 2 or more, L₁ to L₃ may be each independently a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, embodiments of the present disclosure are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate) (Bebq₂), 9,10-di(naphthalen-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), or a mixture thereof.

The electron transport region ETR may include at least one selected from among Compound ET1 to Compound ET36 below:

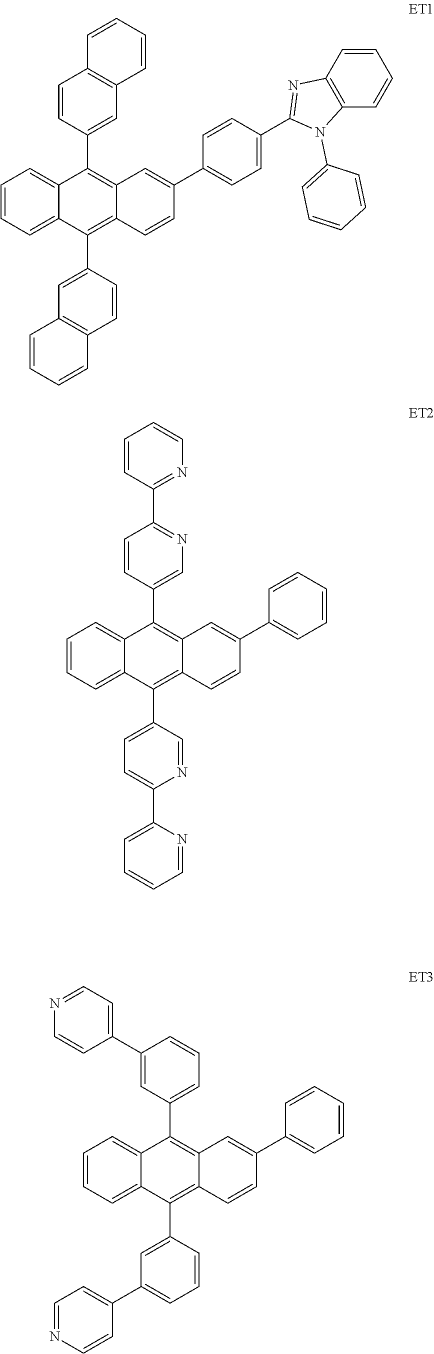 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

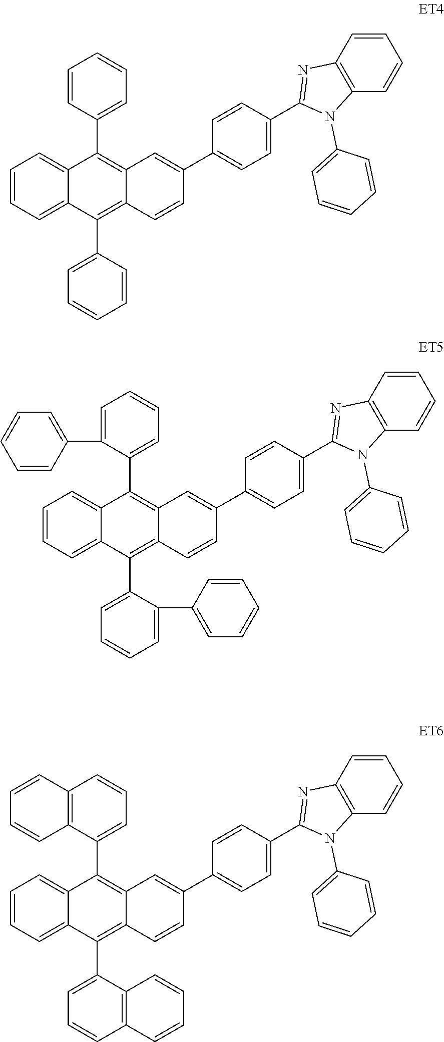 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

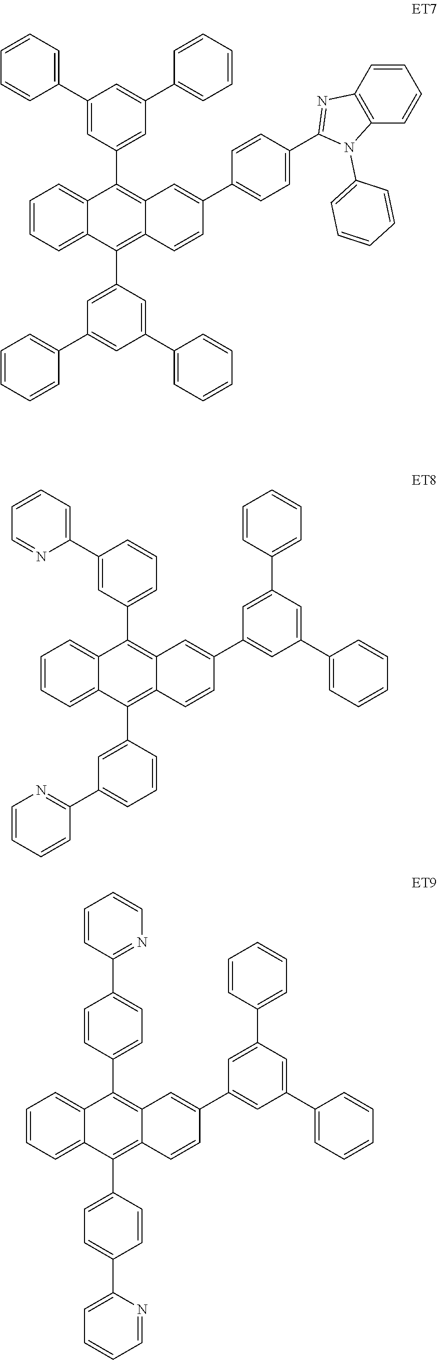 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

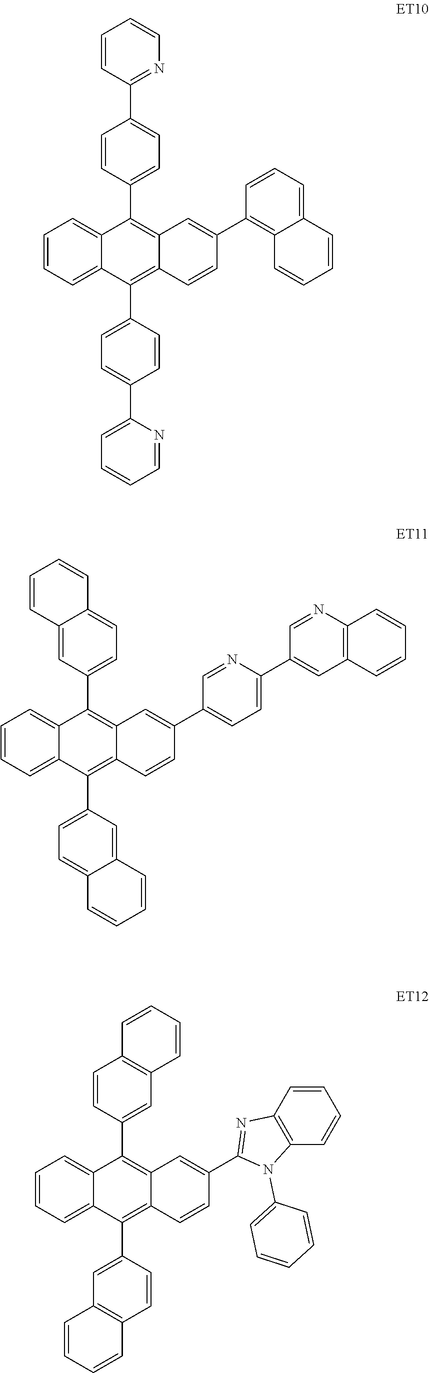 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

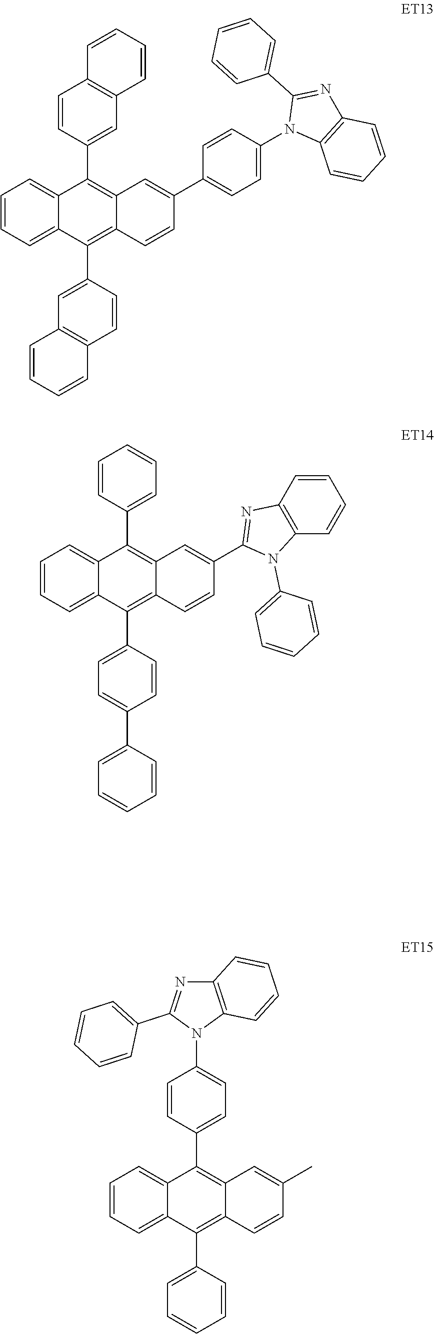 [SMILES: c1ccc(-c2nc3ccccc3n2-c2ccc(-c3ccc4c(-c5ccc6ccccc6c5)c5ccccc5c(-c5ccc6ccccc6c5)c4c3)cc2)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

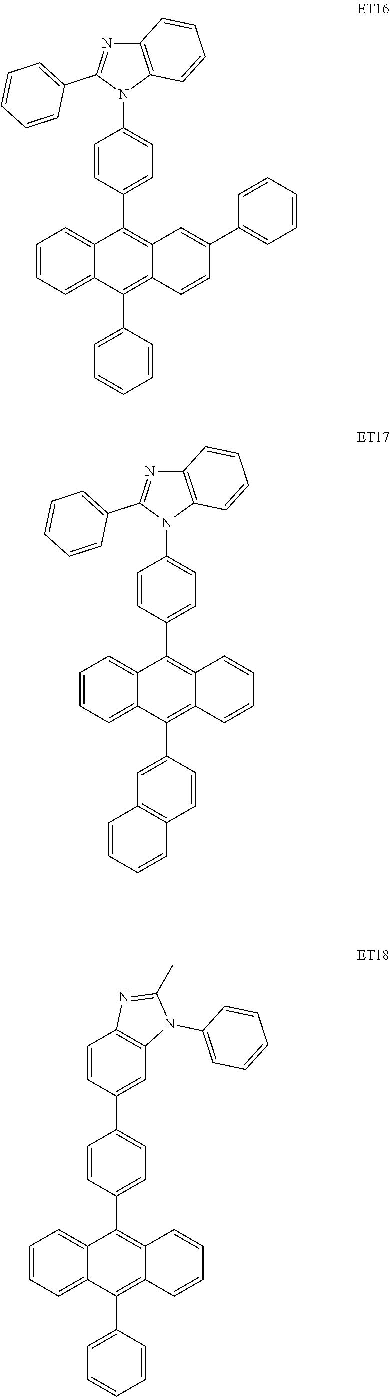 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

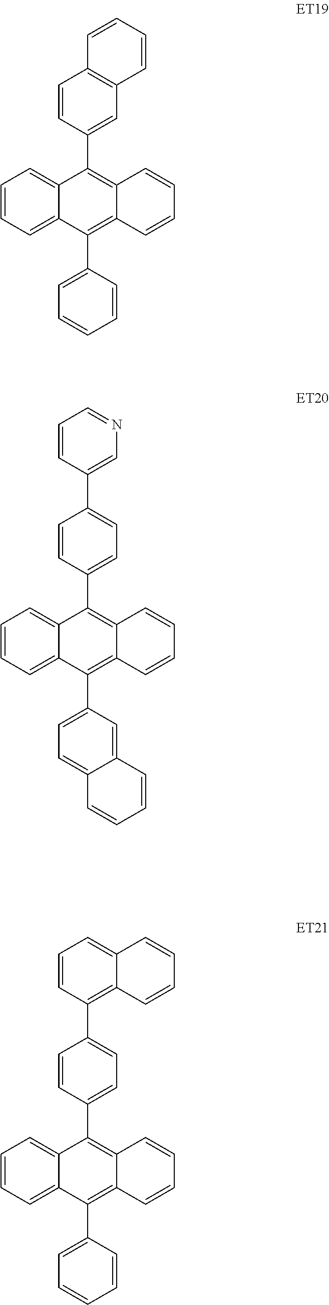

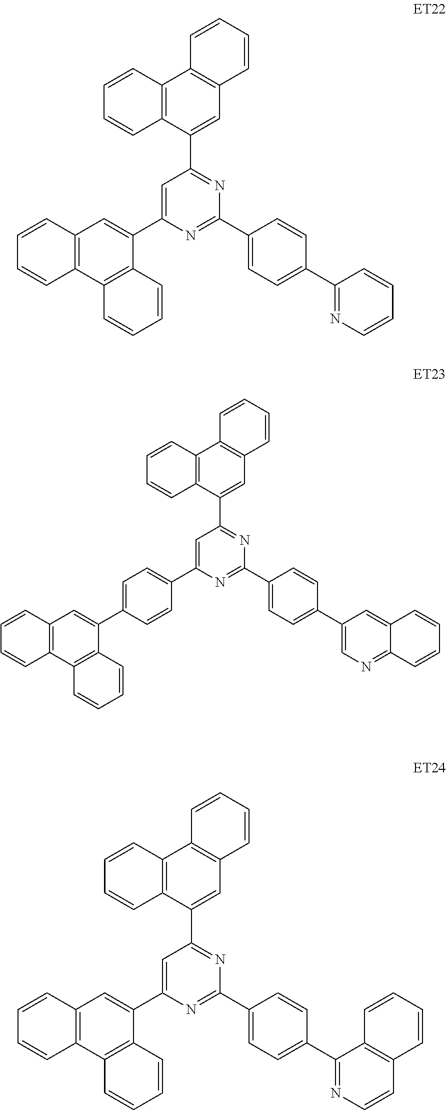 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

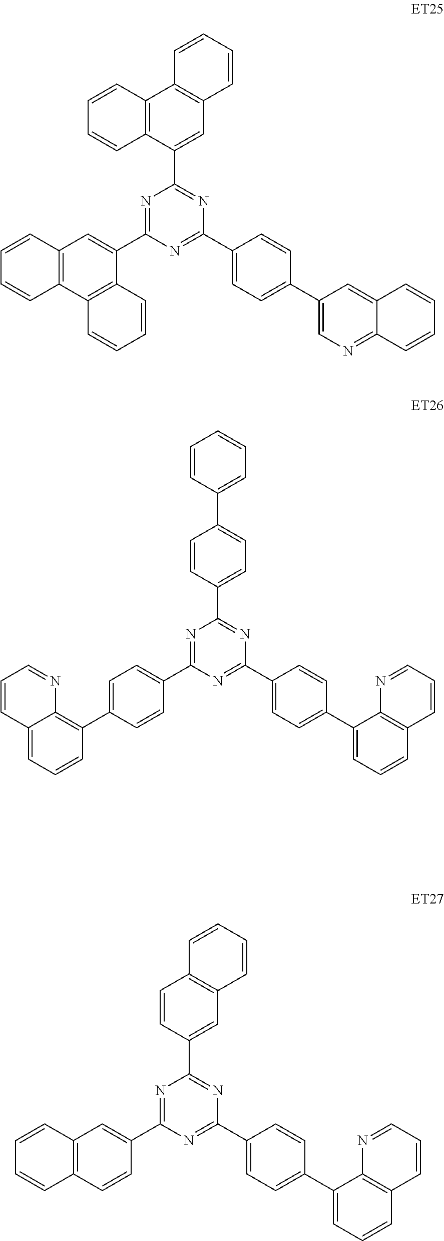 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

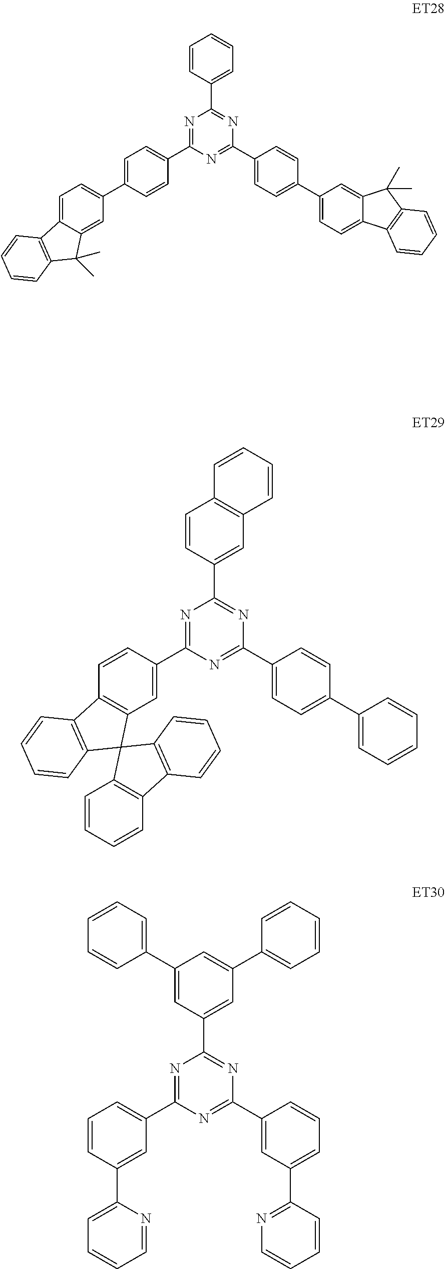 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3nc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

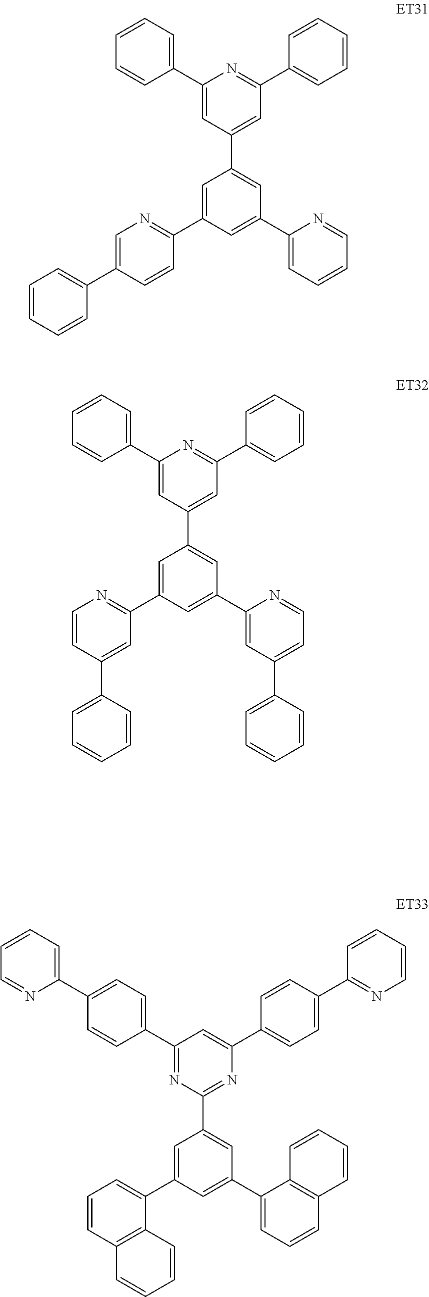 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

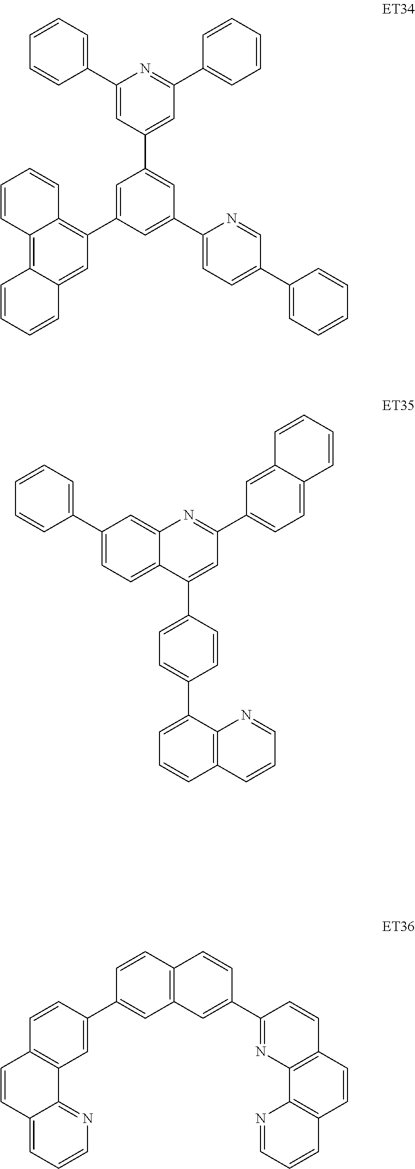 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)cc12]

In some embodiments, the electron transport regions ETR may include a metal halide such as LiF, NaCl, CsF, RbCl, RbI, CuI, and/or KI, a lanthanide metal such as Yb, and/or a co-deposited material of the metal halide and the lanthanide metal. For example, the electron transport region ETR may include KI:Yb, RbI:Yb, LiF:Yb, etc. as a co-deposited material. In some embodiments, the electron transport region ETR may be formed using a metal oxide such as Li₂O and/or BaO, and/or 8-hydroxyl-lithium quinolate (Liq), etc., but embodiments of the present disclosure are not limited thereto. The electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, and/or a metal stearate.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the above-described materials, but embodiments of the present disclosure are not limited thereto.

The electron transport region ETR may include the above-described compounds of the hole transport region in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. If the thickness of the electron transport layer ETL satisfies the aforementioned range, suitable or satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. If the thickness of the electron injection layer EIL satisfies the above-described range, suitable or satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but embodiments of the present disclosure are not limited thereto. For example, when the first electrode EL1 is an anode, the second electrode EL2 may be a cathode, and when the first electrode EL1 is a cathode, the second electrode EL2 may be an anode. The second electrode may include at least one selected from among Ag, Mg, Cu, Al, Pt, Pd, Au, Ni, Nd, Ir, Cr, Li, Ca, LiF, Mo, Ti, W, In, Sn, Zn, a compound of two or more thereof, a mixture of two or more thereof, and an oxide thereof.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, Yb, W, and/or a compound or mixture including these (e.g., AgMg, AgYb, and/or MgYb). In some embodiments, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, and/or the like.

In some embodiments, the second electrode EL2 may be connected with an auxiliary electrode. If the second electrode EL2 is connected with the auxiliary electrode, the resistance of the second electrode EL2 may be decreased.

In some embodiments, a capping layer CPL may further be on the second electrode EL2 of the light emitting element ED of an embodiment. The capping layer CPL may include a multilayer or a single layer.

In an embodiment, the capping layer CPL may be an organic layer and/or an inorganic layer. For example, when the capping layer CPL contains an inorganic material, the inorganic material may include an alkaline metal compound (for example, LiF), an alkaline earth metal compound (for example, MgF₂), SiON, SiNₓ, SiO_{y}, etc.

For example, when the capping layer CPL contains an organic material, the organic material may include α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), etc., and/or may include an epoxy resin, and/or an acrylate such as a methacrylate. However, embodiments of the present disclosure are not limited thereto, and the capping layer CPL may include at least one selected from among Compounds P1 to P5 below:

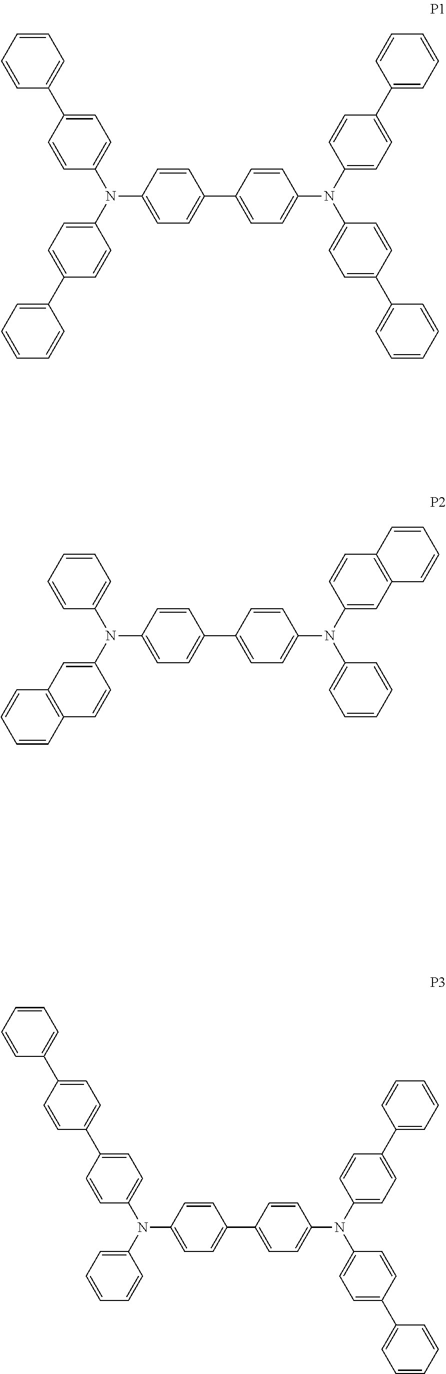 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

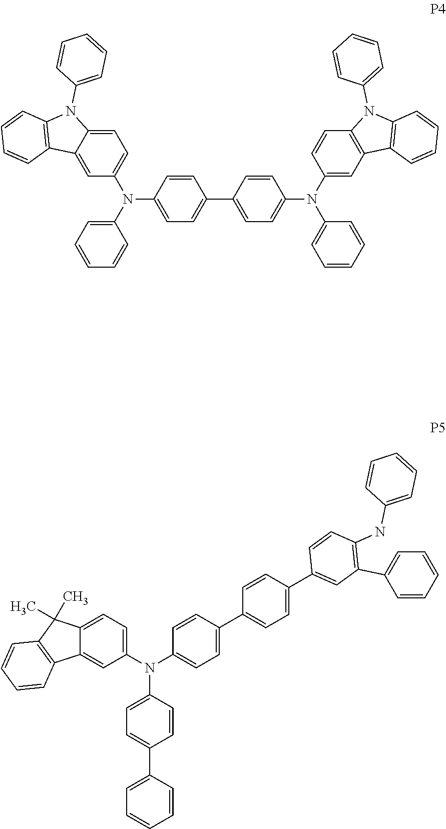 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc(-c5ccc(Nc6ccccc6)c(-c6ccccc6)c5)cc4)cc3)ccc21]

In some embodiments, the refractive index of the capping layer CPL may be about 1.6 or more. For example, the refractive index of the capping layer CPL may be about 1.6 or more with respect to light in a wavelength range of about 550 nm to about 660 nm.

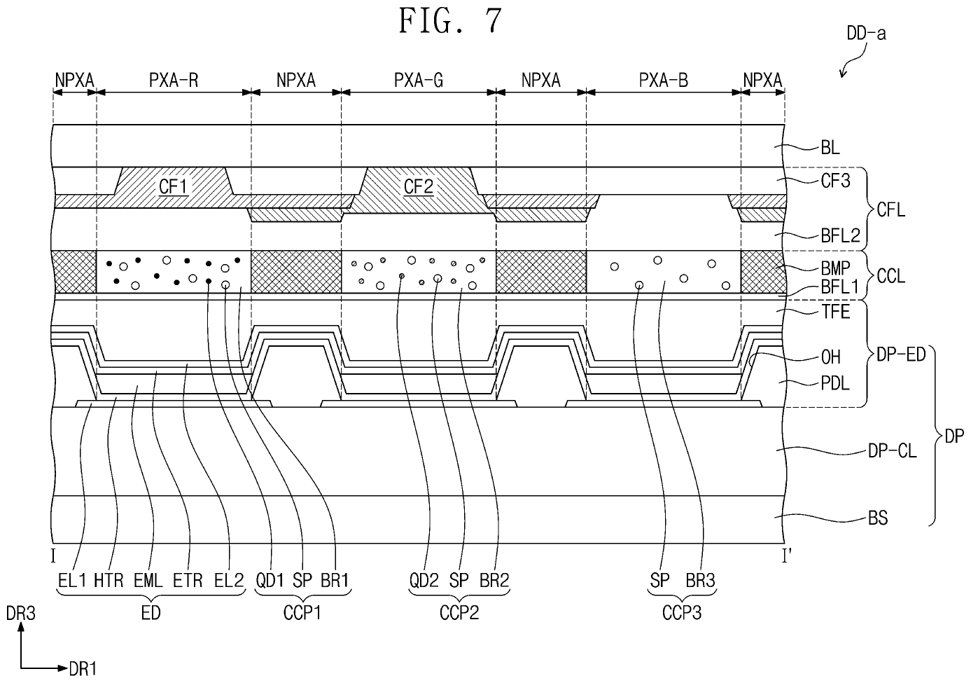

Each of FIGS. 7 to 10 is a cross-sectional view of a display device according to an embodiment of the present disclosure. Hereinafter, in describing the display devices of embodiments with reference to FIGS. 7 to 10, the duplicated features which have been described with respect to FIGS. 1 to 6 are not described again, but their differences will be mainly described.

Referring to FIG. 7, the display device DD-a according to an embodiment may include a display panel DP including a display element layer DP-ED, a light control layer CCL on the display panel DP, and a color filter layer CFL.

In an embodiment illustrated in FIG. 7, the display panel DP may include a base layer BS, a circuit layer DP-CL provided on the base layer BS, and the display element layer DP-ED, and the display element layer DP-ED may include a light emitting element ED.

The light emitting element ED may include a first electrode EL1, a hole transport region HTR on the first electrode EL1, an emission layer EML on the hole transport region HTR, an electron transport region ETR on the emission layer EML, and a second electrode EL2 on the electron transport region ETR. The structures of the light emitting elements ED of FIGS. 3 to 6 as described above may be equally applied to the structure of the light emitting element ED illustrated in FIG. 7.

The hole transport region HTR of the light emitting element ED included in the display device DD-a according to an embodiment may include the above-described amine compound of an embodiment.

Referring to FIG. 7, the emission layer EML may be in an opening OH defined in a pixel defining film PDL. For example, the emission layer EML which is divided by the pixel defining film PDL and provided corresponding to each light emitting regions PXA-R, PXA-G, and PXA-B may emit light in the same wavelength range. In the display device DD-a of an embodiment, the emission layer EML may emit blue light. In some embodiments, the emission layer EML may be provided as a common layer in the entire light emitting regions PXA-R, PXA-G, and PXA-B.

The light control layer CCL may be on the display panel DP. The light control layer CCL may include a light conversion body. The light conversion body may be a quantum dot, a phosphor, and/or the like. The light conversion body may emit provided light by converting the wavelength thereof. For example, the light control layer CCL may include a layer containing the quantum dot and/or a layer containing the phosphor.

The light control layer CCL may include a plurality of light control parts CCP1, CCP2 and CCP3. The light control parts CCP1, CCP2, and CCP3 may be spaced apart from each other.

Referring to FIG. 7, divided patterns BMP may be between the light control parts CCP1, CCP2 and CCP3 which are spaced apart from each other, but embodiments of the present disclosure are not limited thereto. FIG. 7 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts a first color light provided from the light emitting element ED into a second color light, a second light control part CCP2 containing a second quantum dot QD2 which converts the first color light into a third color light, and a third light control part CCP3 which transmits the first color light.

In an embodiment, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, the first quantum dot QD1 may be a red quantum dot, and the second quantum dot QD2 may be a green quantum dot. The same as described above may be applied with respect to the quantum dots QD1 and QD2.

In some embodiments, the light control layer CCL may further include a scatterer SP (e.g., a light scatterer SP). The first light control part CCP1 may include the first quantum dot QD1 and the scatterer SP, the second light control part CCP2 may include the second quantum dot QD2 and the scatterer SP, and the third light control part CCP3 may not include any quantum dot but include the scatterer SP.

The scatterer SP may be inorganic particles. For example, the scatterer SP may include at least one of TiO₂, ZnO, Al₂O₃, SiO₂, or hollow sphere silica. The scatterer SP may include any one selected from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3. The base resins BR1, BR2, and BR3 are media in which the quantum dots QD1 and QD2 and the scatterer SP are dispersed, and may be formed of various suitable resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (which may also be referred to herein as ‘moisture/oxygen’). The barrier layer BFL1 may be on the light control parts CCP1, CCP2, and CCP3 to block or reduce exposure of the light control parts CCP1, CCP2 and CCP3 to moisture/oxygen. In some embodiments, the barrier layer BFL1 may cover the light control parts CCP1, CCP2, and CCP3. In some embodiments, the barrier layer BFL2 may be provided between the light control parts CCP1, CCP2, and CCP3 and the filters CF1, CF2, and CF3.

The barrier layers BFL1 and BFL2 may include at least one inorganic layer. In some embodiments, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. In some embodiments, the barrier layers BFL1 and BFL2 may further include an organic film. The barrier layers BFL1 and BFL2 may be formed of a single layer or a plurality of layers.

In the display device DD of an embodiment, the color filter layer CFL may be on the light control layer CCL. For example, the color filter layer CFL may be directly on the light control layer CCL. In this case, the barrier layer BFL2 may be omitted.

The color filter layer CFL may include filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment and/or dye. The first filter CF1 may include a red pigment and/or dye, the second filter CF2 may include a green pigment and/or dye, and the third filter CF3 may include a blue pigment and/or dye. Embodiments of the present disclosure are not limited thereto, and the third filter CF3 may not include a pigment or dye. The third filter CF3 may include a polymeric photosensitive resin and may not include a pigment or dye. The third filter CF3 may be transparent. The third filter CF3 may be formed of a transparent photosensitive resin.

Furthermore, in an embodiment, the first filter CF1 and the second filter CF2 may be a yellow filter. The first filter CF1 and the second filter CF2 may not be separated but be provided as one filter. The first to third filters CF1, CF2, and CF3 may correspond to the red light emitting region PXA-R, the green light emitting region PXA-G, and the blue light emitting region PXA-B, respectively.

In some embodiments, the color filter layer CFL may include a light shielding part BM. The color filter layer CFL may include a light shielding part BM overlapping at the boundaries of neighboring filters CF1, CF2, and CF3. The light shielding part BM may be a black matrix. The light shielding part BM may include an organic light shielding material and/or an inorganic light shielding material containing a black pigment and/or dye. The light shielding part BM may separate boundaries between the adjacent filters CF1, CF2, and CF3. In some embodiments, in an embodiment, the light shielding part BM may be formed of a blue filter.

A base substrate BL may be on the color filter layer CFL. The base substrate BL may be a member which provides a base surface in which the color filter layer CFL, the light control layer CCL, and the like are located. The base substrate BL may be a glass substrate, a metal substrate, a plastic substrate, etc. However, embodiments of the present disclosure are not limited thereto, and the base substrate BL may be an inorganic layer, an organic layer, or a composite material layer. In some embodiments, the base substrate BL may be omitted.

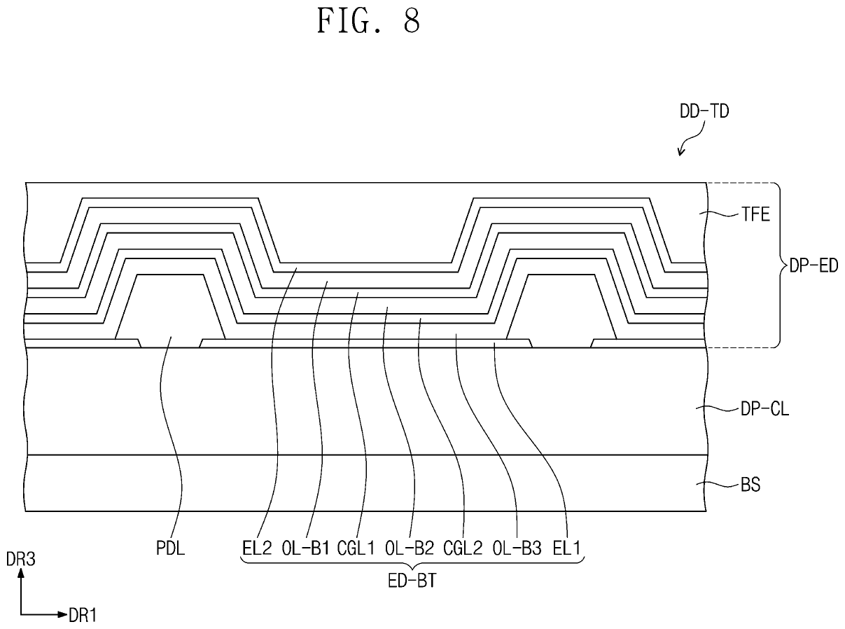

FIG. 8 is a cross-sectional view illustrating a portion of a display device according to an embodiment of the present disclosure. FIG. 8 illustrates a cross-sectional view of a part corresponding to the display panel DP of FIG. 7. In the display device DD-TD of an embodiment, the light emitting element ED-BT may include a plurality of light emitting structures OL-B1, OL-B2, and OL-B3. The light emitting element ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 7) and a hole transport region HTR and an electron transport region ETR with the emission layer EML (FIG. 7) located therebetween.

In some embodiments, the light emitting element ED-BT included in the display device DD-TD of an embodiment may be a light emitting element having a tandem structure and including a plurality of emission layers.

In an embodiment illustrated in FIG. 8, all light beams respectively emitted from the light emitting structures OL-B1, OL-B2, and OL-B3 may be blue light. However, embodiments of the present disclosure are not limited thereto, and the light beams respectively emitted from the light emitting structures OL-B1, OL-B2, and OL-B3 may have wavelength ranges different from each other. For example, the light emitting element ED-BT including the plurality of light emitting structures OL-B1, OL-B2, and OL-B3 which emit light beams having wavelength ranges different from each other may emit white light.

Charge generation layers CGL1 and CGL2 may be respectively between two of the neighboring light emitting structures OL-B1, OL-B2, and OL-B3. The charge generation layers CGL1 and CGL2 may include a p-type charge generation layer and/or an n-type charge generation layer.

At least one of the light emitting structures OL-B1, OL-B2, or OL-B3 included in the display device DD-TD of an embodiment may contain the above-described amine compound of an embodiment.

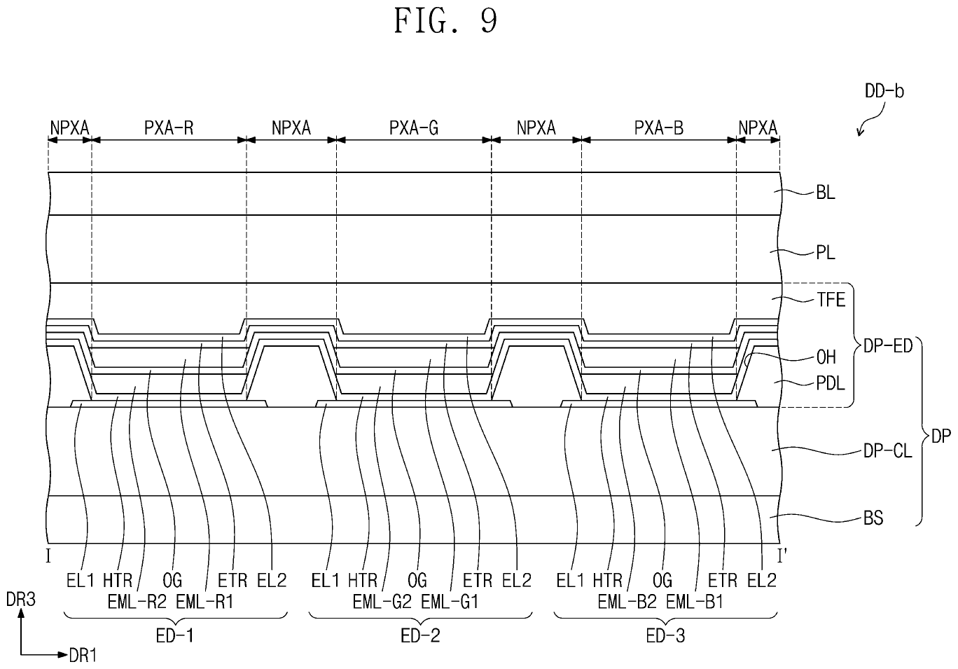

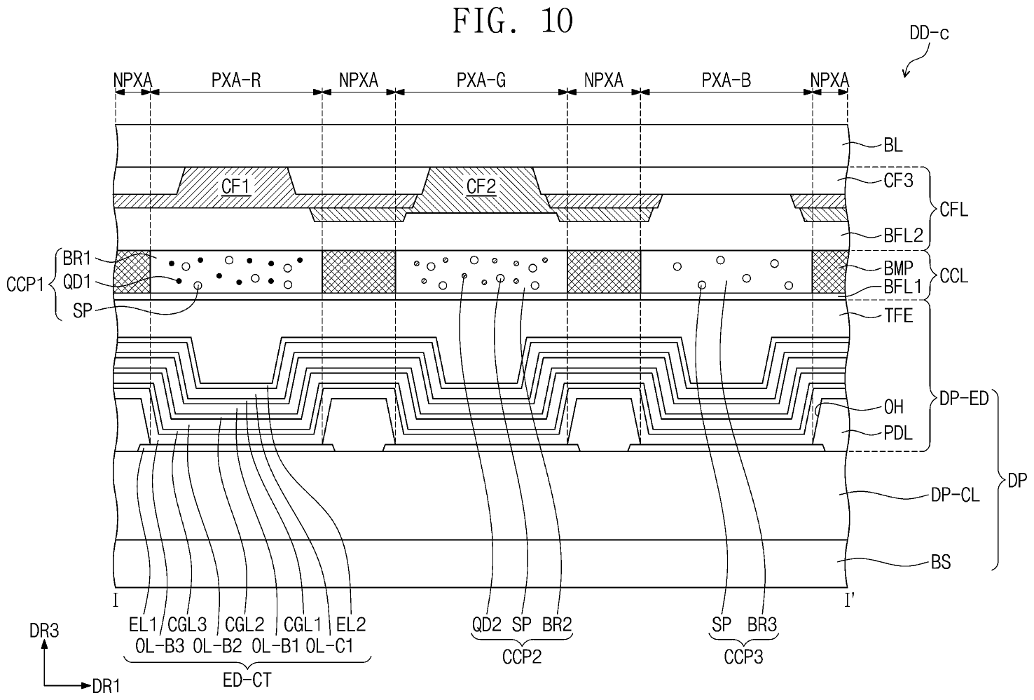

Referring to FIG. 9, the display device DD-b according to an embodiment may include light emitting elements ED-1, ED-2, and ED-3 in which two emission layers are stacked. Compared with the display device DD of an embodiment illustrated in FIG. 2, an embodiment illustrated in FIG. 9 has a difference in that the first to third light emitting elements ED-1, ED-2, and ED-3 each include two emission layers stacked in the thickness direction. In each of the first to third light emitting elements ED-1, ED-2, and ED-3, the two emission layers may emit light in the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. An emission auxiliary part OG may be between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. For example, the emission auxiliary part OG may include an electron transport region, a charge generation layer, and a hole transport region that are sequentially stacked. The emission auxiliary part OG may be provided as a common layer in the whole of the first to third light emitting elements ED-1, ED-2, and ED-3. However, embodiments of the present disclosure are not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may be between the hole transport region HTR and the emission auxiliary part OG. The second red emission layer EML-R2, the second green emission layer EML-G2, and the second blue emission layer EML-B2 may be between the emission auxiliary part OG and the electron transport region ETR.

In some embodiments, the first light emitting element ED-1 may include the first electrode EL1, the hole transport region HTR, the second red emission layer EML-R2, the emission auxiliary part OG, the first red emission layer EML-R1, the electron transport region ETR, and the second electrode EL2 that are sequentially stacked. The second light emitting element ED-2 may include the first electrode EL1, the hole transport region HTR, the second green emission layer EML-G2, the emission auxiliary part OG, the first green emission layer EML-G1, the electron transport region ETR, and the second electrode EL2 that are sequentially stacked. The third light emitting element ED-3 may include the first electrode EL1, the hole transport region HTR, the second blue emission layer EML-B2, the emission auxiliary part OG, the first blue emission layer EML-B1, the electron transport region ETR, and the second electrode EL2 that are sequentially stacked.

In some embodiments, an optical auxiliary layer PL may be on the display element layer DP-ED. The optical auxiliary layer PL may include a polarizing layer. The optical auxiliary layer PL may be on the display panel DP and control reflected light in the display panel DP due to external light. The optical auxiliary layer PL in the display device according to some embodiments may be omitted.

Unlike FIGS. 8 and 9, FIG. 10 illustrates that a display device DD-c includes four light emitting structures OL-B1, OL-B2, OL-B3, and OL-C1. A light emitting element ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. Charge generation layers CGL1, CGL2, and CGL3 may be between the first to fourth light emitting structures OL-B1, OL-B2, OL-B3, and OL-C1. Among the four light emitting structures, the first to third light emitting structures OL-B1, OL-B2, and OL-B3 may emit blue light, and the fourth light emitting structure OL-C1 may emit green light. However, embodiments of the present disclosure are not limited thereto, and the first to fourth light emitting structures OL-B1, OL-B2, OL-B3, and OL-C1 may emit light beams in different wavelength regions.

The charge generation layers CGL1, CGL2, and CGL3 between adjacent light emitting structures OL-B1, OL-B2, OL-B3, and OL-C1 may include a p-type charge generation layer and/or an n-type charge generation layer.

At least one selected from among the light emitting structures OL-B1, OL-B2, OL-B3, and OL-C1 included in the display device DD-c of an embodiment may include the above-described amine compound of an embodiment.

The light emitting element ED according to an embodiment of the present disclosure may include the above-described amine compound of an embodiment in at least one functional layer between the first electrode EL1 and the second electrode EL2, thereby exhibiting improved luminous efficiency and service life characteristics. The light emitting element ED according to an embodiment may include the above-described amine compound of an embodiment in at least one of the hole transport region HTR, the emission layer EML, or the electron transport region ETR between the first electrode EL1 and the second electrode EL2, or in a capping layer CPL.

For example, the amine compound according to an embodiment may be included in the hole transport region HTR of the light emitting element ED of an embodiment, and the light emitting element of an embodiment may exhibit excellent luminous efficiency and long service life characteristics.

The above-described amine compound of an embodiment may include a phenanthrene moiety directly linked to the nitrogen atom, and also include a naphthalene moiety, a phenanthrene moiety, a dibenzoheterole moiety, and/or a carbazole moiety linked via a linker to or directly linked to the nitrogen atom, thereby exhibiting high efficiency and increased service life characteristics.

The amine compound of an embodiment may include a benzonaphthofuran moiety and a dibenzofuran moiety linked via a linker to or directly linked to the nitrogen atom, thereby high efficiency and increased service life characteristics.

Hereinafter, with reference to Examples and Comparative Examples, an amine compound according to an embodiment of the present disclosure and a light emitting element of an embodiment of the present disclosure will be described in more detail. In addition, Examples described below are only illustrations to assist the understanding of the subject matter of the present disclosure, and the scope of the present disclosure is not limited thereto.

### Examples

### 1. Synthesis of Amine Compound

First, a synthetic method of an amine compound according to the present embodiment will be described in more detail by illustrating the synthetic methods of Compounds A1, A11, A23, A32, A65, A84, B6, B25, B74, B1118, B1129, C23, C50, C85, C133, D2, D26, D41 and C101. Also, in the following descriptions, the synthetic method of the amine compound is provided as an example, but the synthetic method according to an embodiment of the present disclosure is not limited to Examples below.

### (1) Synthesis of Compound A1

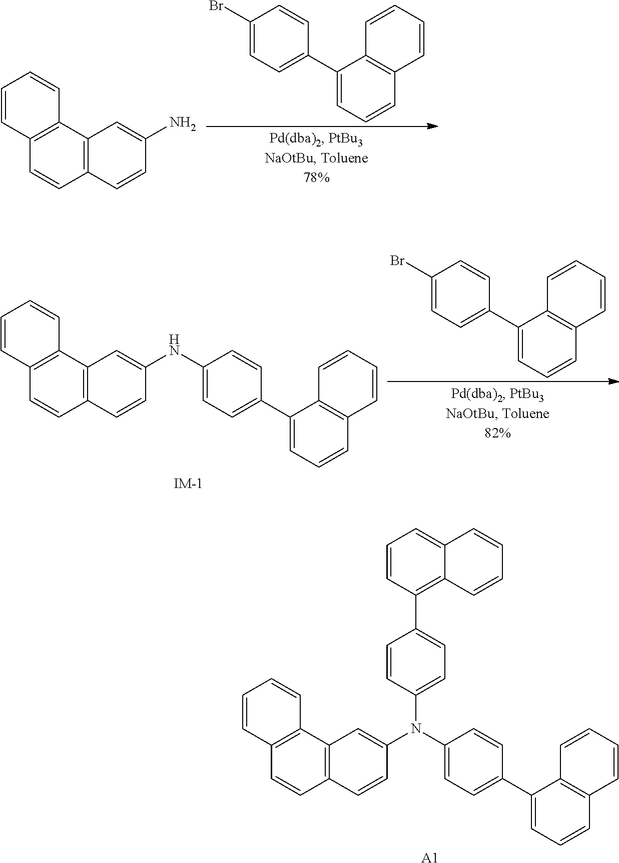

### 1) Synthesis of Intermediate Compound IM-1

In an Ar atmosphere, in a 500 mL three-neck flask, 3-aminophenanthrene (10.00 g, 51.7 mmol), Pd(dba)₂ (0.89 g, 0.03 equivalent (“equiv”), 1.6 mmol), NaOtBu (4.97 g, 1.0 equiv, 51.7 mmol), toluene (259 mL), 1-(4-bromophenyl)naphthalene (16.11 g, 1.1 equiv, 56.9 mmol), and P^{t}Bu₃ (1.05 g, 0.1 equiv, 5.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate Compound IM-1 (15.96 g, yield 78%).

By measuring utilizing fast atom bombardment-mass spectrometry (FAB-MS), a mass number of m/z=395 was observed by molecular ion peak, thereby identifying Intermediate Compound IM-1.

### 2) Synthesis of Compound A1

In an Ar atmosphere, in a 300 mL three-neck flask, Intermediate Compound IM-1 (10.00 g, 25.3 mmol), Pd(dba)₂ (0.44 g, 0.03 equiv, 0.8 mmol), NaO^{t}Bu (4.86 g, 2.0 equiv, 50.6 mmol), toluene (126 mL), 1-(4-bromophenyl)naphthalene (7.88 g, 1.1 equiv, 27.8 mmol), and P^{t}Bu₃ (0.51 g, 0.1 equiv, 2.5 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound A1 (12.39 g, yield 82%).

By measuring utilizing FAB-MS, a mass number of m/z=597 was observed by molecular ion peak, thereby identifying Compound A1.

### (2) Synthesis of Compound A11

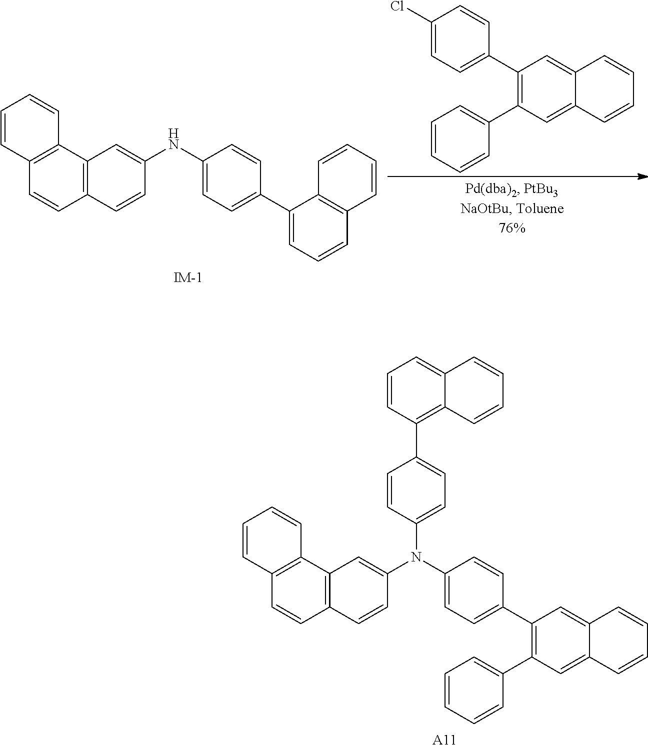

### 1) Synthesis of Compound A11

In an Ar atmosphere, in a 300 mL three-neck flask, Intermediate Compound IM-1 (10.00 g, 25.3 mmol), Pd(dba)₂ (0.44 g, 0.03 equiv, 0.8 mmol), NaO^{t}Bu (4.86 g, 2.0 equiv, 50.6 mmol), toluene (126 mL), 2-(4-chlorophenyl)-3-phenylnaphthalene (8.76 g, 1.1 equiv, 27.8 mmol), and P^{t}Bu₃ (0.51 g, 0.1 equiv, 2.5 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound A11 (12.95 g, yield 76%).

By measuring utilizing FAB-MS, a mass number of m/z=597 was observed by molecular ion peak, thereby identifying Compound A11.

### (3) Synthesis of Compound A23

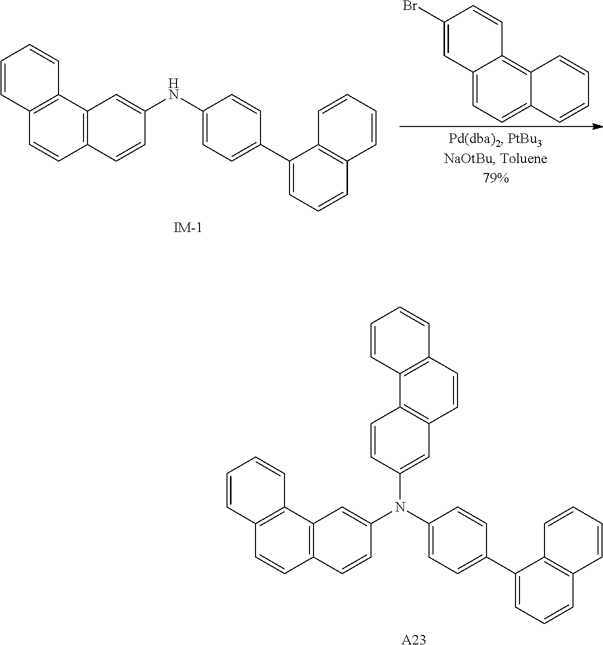

### 1) Synthesis of Compound A23

In an Ar atmosphere, in a 300 mL three-neck flask, Intermediate Compound IM-1 (10.00 g, 25.3 mmol), Pd(dba)₂ (0.44 g, 0.03 equiv, 0.8 mmol), NaO^{t}Bu (4.86 g, 2.0 equiv, 50.6 mmol), toluene (126 mL), 2-bromophenanthrene (7.15 g, 1.1 equiv, 27.8 mmol), and P^{t}Bu₃ (0.51 g, 0.1 equiv, 2.5 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound A23 (11.42 g, yield 79%).

By measuring utilizing FAB-MS, a mass number of m/z=571 was observed by molecular ion peak, thereby identifying Compound A23.

### (4) Synthesis of Compound A32

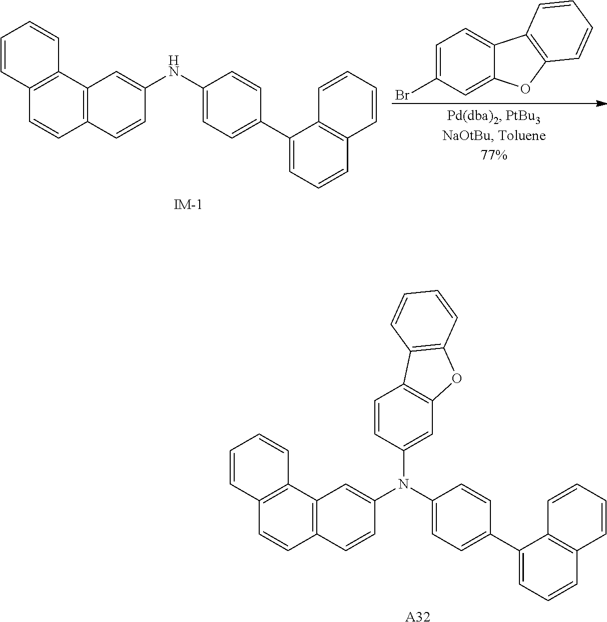

### 1) Synthesis of Compound A32

In an Ar atmosphere, in a 300 mL three-neck flask, Intermediate Compound IM-1 (10.00 g, 25.3 mmol), Pd(dba)₂ (0.44 g, 0.03 equiv, 0.8 mmol), NaO^{t}Bu (4.86 g, 2.0 equiv, 50.6 mmol), toluene (126 mL), 3-bromodibenzofuran (6.87 g, 1.1 equiv, 27.8 mmol), and P^{t}Bu₃ (0.51 g, 0.1 equiv, 2.5 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound A32 (10.94 g, yield 77%).

By measuring utilizing FAB-MS, a mass number of m/z=561 was observed by molecular ion peak, thereby identifying Compound A32.

### (5) Synthesis of Compound A65

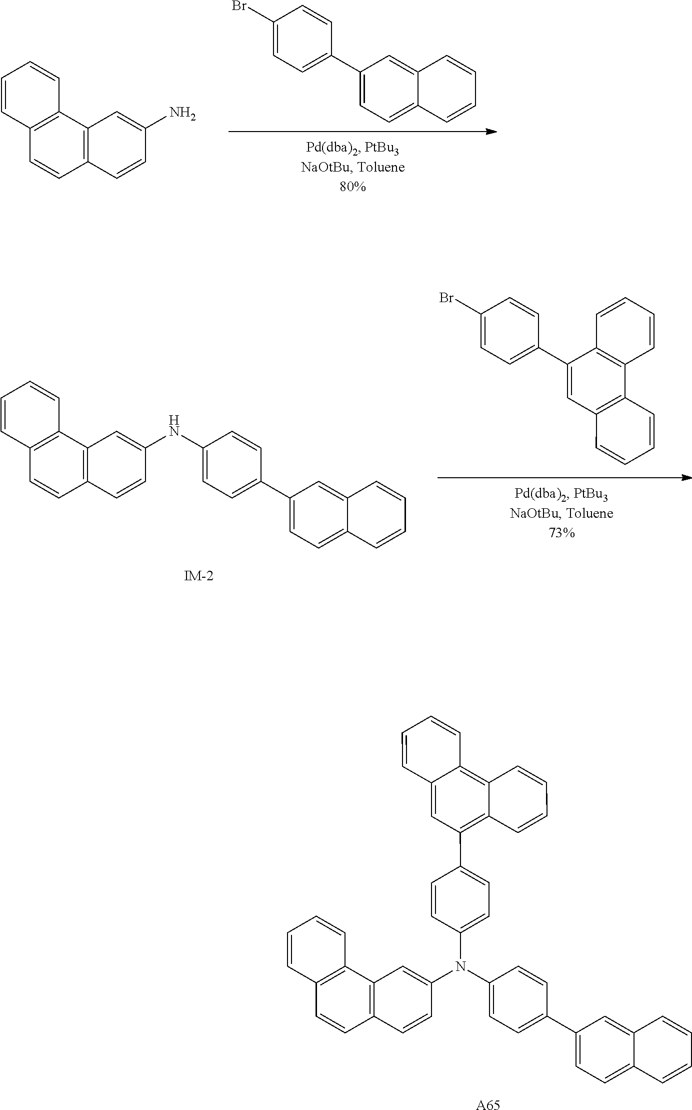

### 1) Synthesis of Intermediate Compound IM-2

In an Ar atmosphere, in a 500 mL three-neck flask, 3-aminophenanthrene (10.00 g, 51.7 mmol), Pd(dba)₂ (0.89 g, 0.03 equiv, 1.6 mmol), NaO^{t}Bu (4.97 g, 1.0 equiv, 51.7 mmol), toluene (259 mL), 2-(4-bromophenyl)naphthalene (16.11 g, 1.1 equiv, 56.9 mmol), and P^{t}Bu₃ (1.05 g, 0.1 equiv, 5.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate Compound IM-2 (16.37 g, yield 80%).

By measuring utilizing FAB-MS, a mass number of m/z=395 was observed by molecular ion peak, thereby identifying Intermediate Compound IM-2.

### 2) Synthesis of Compound A65

In an Ar atmosphere, in a 300 mL three-neck flask, Intermediate Compound IM-2 (10.00 g, 25.3 mmol), Pd(dba)₂ (0.44 g, 0.03 equiv, 0.8 mmol), NaOtBu (4.86 g, 2.0 equiv, 50.6 mmol), toluene (126 mL), 9-(4-bromophenyl)phenanthrene (9.27 g, 1.1 equiv, 27.8 mmol), and P^{t}Bu₃ (0.51 g, 0.1 equiv, 2.5 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound A65 (11.96 g, yield 73%).

By measuring utilizing FAB-MS, a mass number of m/z=647 was observed by molecular ion peak, thereby identifying Compound A65.

### (6) Synthesis of Compound A84

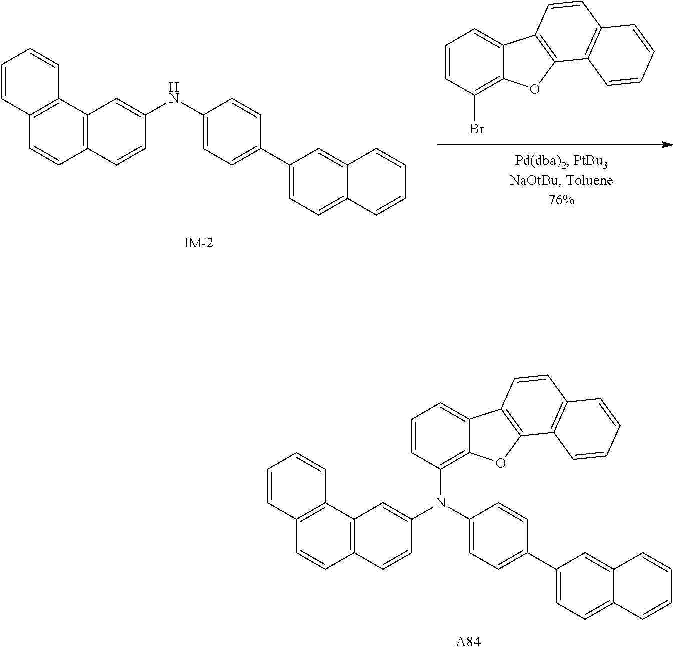

### 1) Synthesis of Compound A84

In an Ar atmosphere, in a 300 mL three-neck flask, Intermediate Compound IM-2 (10.00 g, 25.3 mmol), Pd(dba)₂ (0.44 g, 0.03 equiv, 0.8 mmol), NaO^{t}Bu (4.86 g, 2.0 equiv, 50.6 mmol), toluene (126 mL), 10-bromonaphthobenzofuran (8.26 g, 1.1 equiv, 27.8 mmol), and P^{t}Bu₃ (0.51 g, 0.1 equiv, 2.5 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound A84 (11.76 g, yield 76%).

By measuring utilizing FAB-MS, a mass number of m/z=611 was observed by molecular ion peak, thereby identifying Compound A84.

### (7) Synthesis of Compound B6

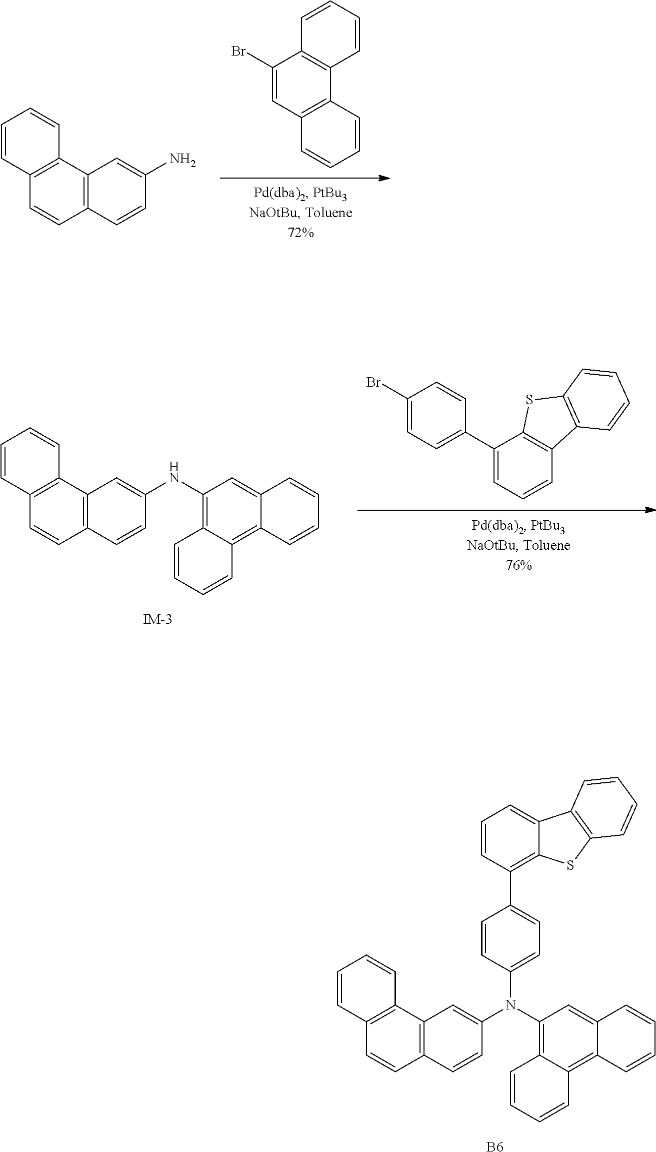

### 1) Synthesis of Intermediate Compound IM-3

In an Ar atmosphere, in a 500 mL three-neck flask, 3-aminophenanthrene (10.00 g, 51.7 mmol), Pd(dba)₂ (0.89 g, 0.03 equiv, 1.6 mmol), NaOtBu (4.97 g, 1.0 equiv, 51.7 mmol), toluene (259 mL), 9-bromophenanthrene (14.64 g, 1.1 equiv, 56.9 mmol), and P^{t}Bu₃ (1.05 g, 0.1 equiv, 5.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate Compound IM-3 (13.77 g, yield 72%).

By measuring utilizing FAB-MS, a mass number of m/z=369 was observed by molecular ion peak, thereby identifying Intermediate Compound IM-3.

### 2) Synthesis of Compound B6

In an Ar atmosphere, in a 300 mL three-neck flask, Intermediate Compound IM-3 (10.00 g, 27.1 mmol), Pd(dba)₂ (0.46 g, 0.03 equiv, 0.8 mmol), NaOtBu (5.20 g, 2.0 equiv, 54.1 mmol), toluene (135 mL), 4-(4-bromophenyl)dibenzothiophene (10.10 g, 1.1 equiv, 29.8 mmol), and P^{t}Bu₃ (0.55 g, 0.1 equiv, 2.7 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound B6 (12.91 g, yield 76%).

By measuring utilizing FAB-MS, a mass number of m/z=627 was observed by molecular ion peak, thereby identifying Compound B6.

### (8) Synthesis of Compound B25

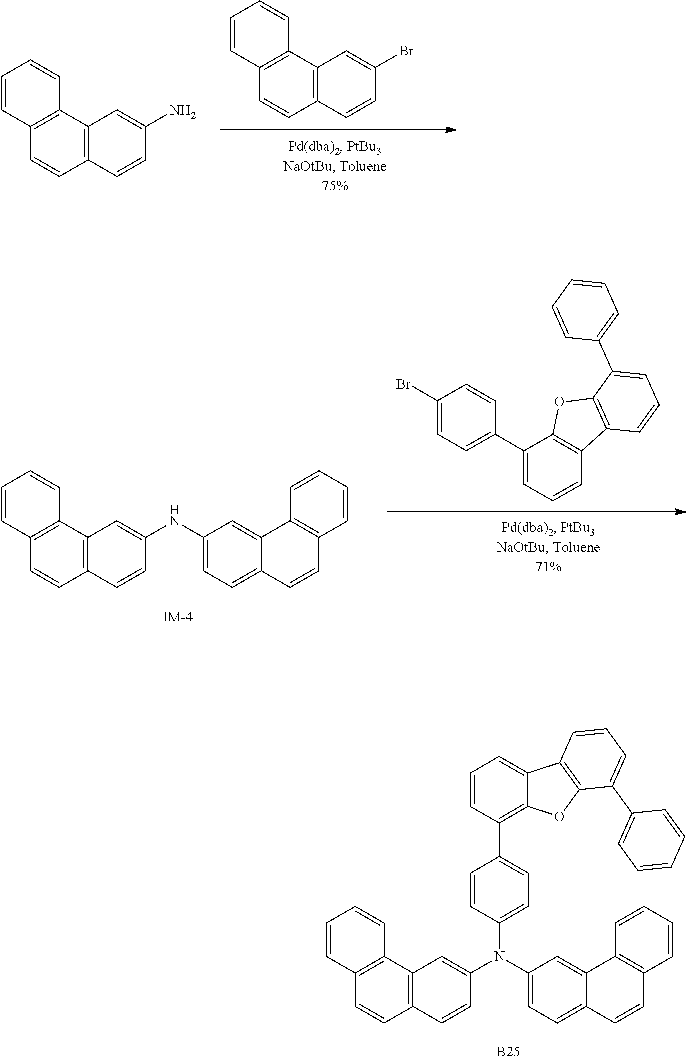

### 1) Synthesis of Intermediate Compound IM-4

In an Ar atmosphere, in a 500 mL three-neck flask, 3-aminophenanthrene (10.00 g, 51.7 mmol), Pd(dba)₂ (0.89 g, 0.03 equiv, 1.6 mmol), NaO^{t}Bu (4.97 g, 1.0 equiv, 51.7 mmol), toluene (259 mL), 3-bromophenanthrene (14.64 g, 1.1 equiv, 56.9 mmol), and P^{t}Bu₃ (1.05 g, 0.1 equiv, 5.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate Compound IM-4 (14.34 g, yield 75%).

By measuring utilizing FAB-MS, a mass number of m/z=369 was observed by molecular ion peak, thereby identifying Intermediate Compound IM-4.

### 2) Synthesis of Compound B25

In an Ar atmosphere, in a 300 mL three-neck flask, Intermediate Compound IM-4 (10.00 g, 27.1 mmol), Pd(dba)₂ (0.46 g, 0.03 equiv, 0.8 mmol), NaOtBu (5.20 g, 2.0 equiv, 54.1 mmol), toluene (135 mL), 4-(4-bromophenyl)-6-phenyldibenzofuran (11.89 g, 1.1 equiv, 29.8 mmol), and P^{t}Bu₃ (0.55 g, 0.1 equiv, 2.7 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound B25 (13.22 g, yield 71%).

By measuring utilizing FAB-MS, a mass number of m/z=687 was observed by molecular ion peak, thereby identifying Compound B25.

### (9) Synthesis of Compound B74

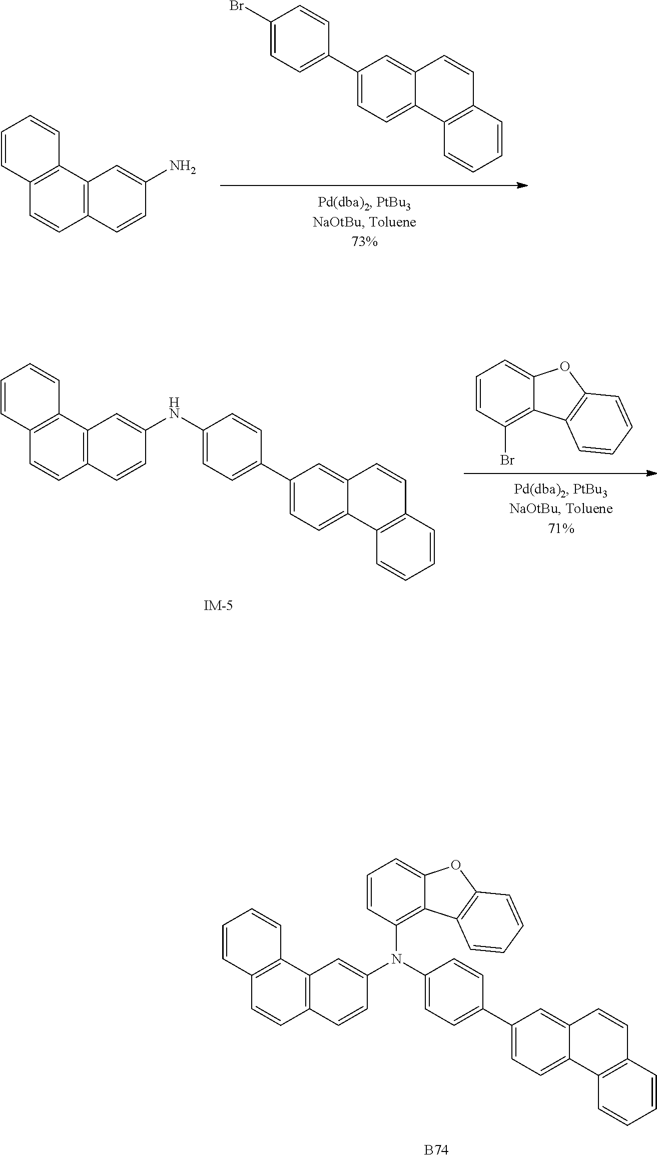

### 1) Synthesis of Intermediate Compound IM-5

In an Ar atmosphere, in a 500 mL three-neck flask, 3-aminophenanthrene (10.00 g, 51.7 mmol), Pd(dba)₂ (0.89 g, 0.03 equiv, 1.6 mmol), NaO^{t}Bu (4.97 g, 1.0 equiv, 51.7 mmol), toluene (259 mL), 2-(4-bromophenyl)phenanthrene (18.97 g, 1.1 equiv, 56.9 mmol), and P^{t}Bu₃ (1.05 g, 0.1 equiv, 5.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate Compound IM-5 (16.83 g, yield 73%).

By measuring utilizing FAB-MS, a mass number of m/z=445 was observed by molecular ion peak, thereby identifying Intermediate Compound IM-5.

### 2) Synthesis of Compound B74

In an Ar atmosphere, in a 300 mL three-neck flask, Intermediate Compound IM-5 (10.00 g, 22.4 mmol), Pd(dba)₂ (0.39 g, 0.03 equiv, 0.7 mmol), NaOtBu (4.31 g, 2.0 equiv, 44.9 mmol), toluene (112 mL), 1-bromodibenzofuran (6.50 g, 1.1 equiv, 24.7 mmol), and P^{t}Bu₃ (0.45 g, 0.1 equiv, 2.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound B74 (9.75 g, yield 71%).

By measuring utilizing FAB-MS, a mass number of m/z=611 was observed by molecular ion peak, thereby identifying Compound B74.

### (10) Synthesis of Compound B118

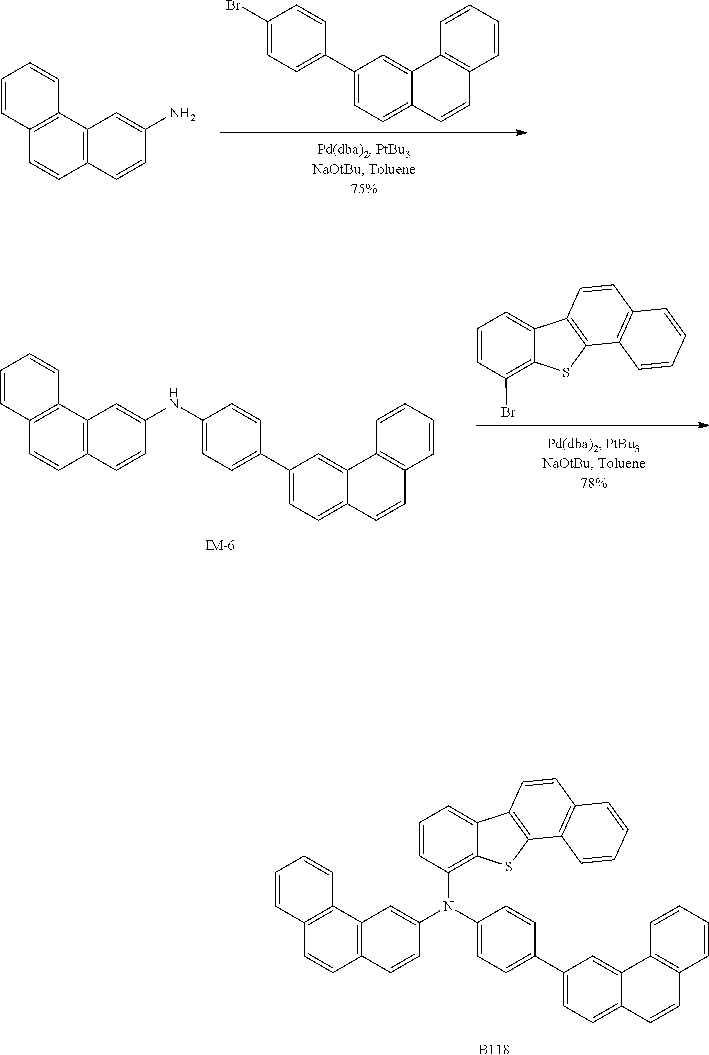

### 1) Synthesis of Intermediate Compound IM-6

In an Ar atmosphere, in a 500 mL three-neck flask, 3-aminophenanthrene (10.00 g, 51.7 mmol), Pd(dba)₂ (0.89 g, 0.03 equiv, 1.6 mmol), NaO^{t}Bu (4.97 g, 1.0 equiv, 51.7 mmol), toluene (259 mL), 3-(4-bromophenyl)phenanthrene (18.97 g, 1.1 equiv, 56.9 mmol), and P^{t}Bu₃ (1.05 g, 0.1 equiv, 5.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate Compound IM-6 (17.29 g, yield 75%).

By measuring utilizing FAB-MS, a mass number of m/z=445 was observed by molecular ion peak, thereby identifying Intermediate Compound IM-6.

### 2) Synthesis of Compound B118

In an Ar atmosphere, in a 300 mL three-neck flask, Intermediate Compound IM-6 (10.00 g, 22.4 mmol), Pd(dba)₂ (0.39 g, 0.03 equiv, 0.7 mmol), NaO^{t}Bu (4.31 g, 2.0 equiv, 44.9 mmol), toluene (112 mL), 10-bromobenzo[b]naphthothiophene (8.13 g, 1.1 equiv, 24.7 mmol), and P^{t}Bu₃ (0.45 g, 0.1 equiv, 2.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound B118 (11.87 g, yield 78%).

By measuring utilizing FAB-MS, a mass number of m/z=677 was observed by molecular ion peak, thereby identifying Compound B118.

### (11) Synthesis of Compound B129

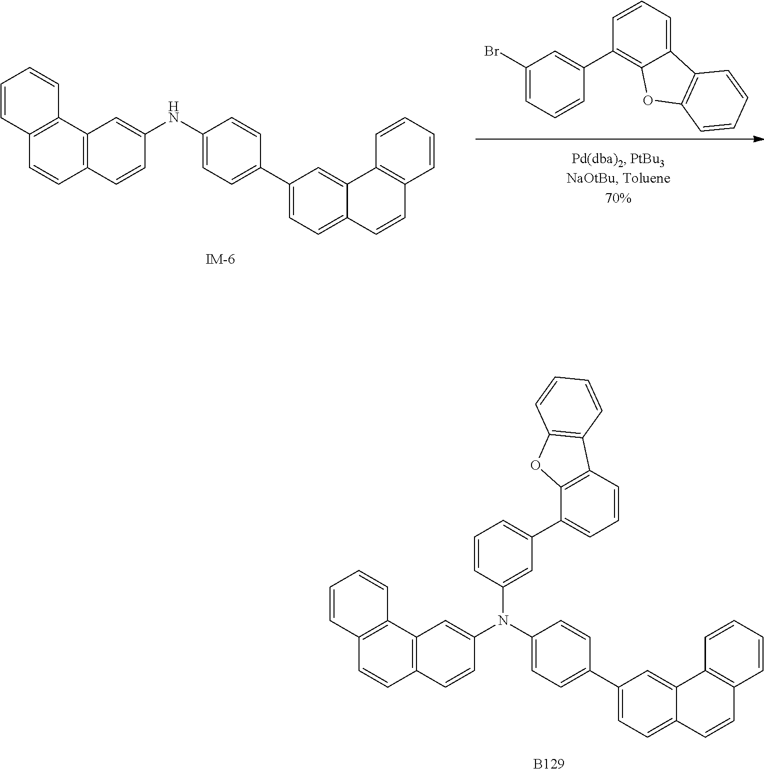

### 1) Synthesis of Compound B129

In an Ar atmosphere, in a 300 mL three-neck flask, Intermediate Compound IM-6 (10.00 g, 22.4 mmol), Pd(dba)₂ (0.39 g, 0.03 equiv, 0.7 mmol), NaOtBu (4.31 g, 2.0 equiv, 44.9 mmol), toluene (112 mL), 4-(3-bromophenyl)dibenzofuran (7.98 g, 1.1 equiv, 24.7 mmol), and P^{t}Bu₃ (0.45 g, 0.1 equiv, 2.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound B129 (10.81 g, yield 70%).

By measuring utilizing FAB-MS, a mass number of m/z=687 was observed by molecular ion peak, thereby identifying Compound B129.

### (12) Synthesis of Compound C23

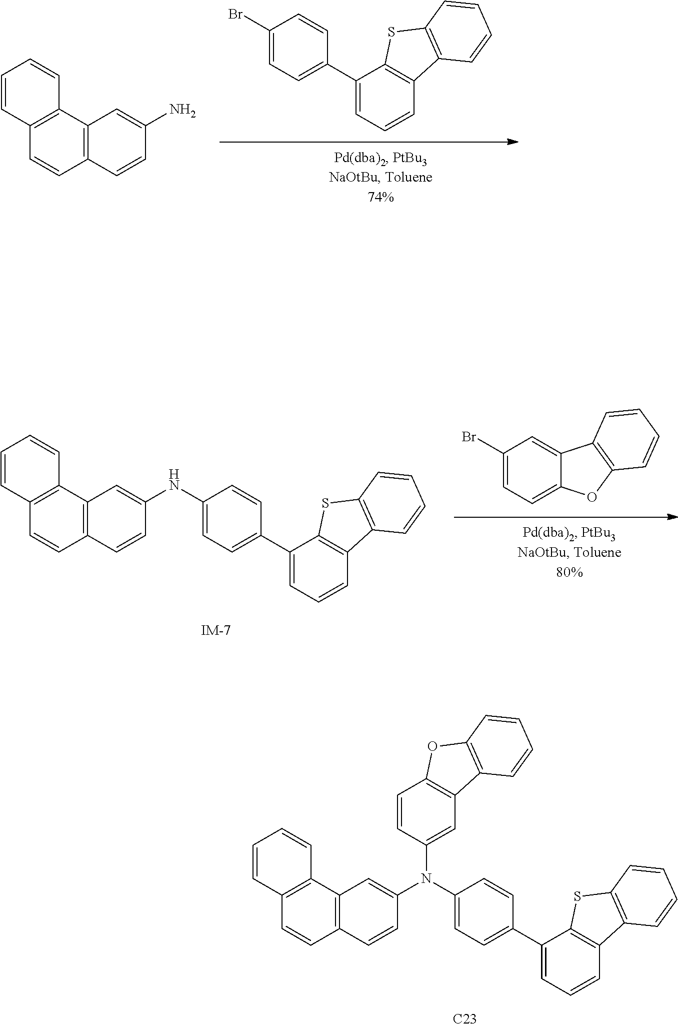

### 1) Synthesis of Intermediate Compound IM-7

In an Ar atmosphere, in a 500 mL three-neck flask, 3-aminophenanthrene (10.00 g, 51.7 mmol), Pd(dba)₂ (0.89 g, 0.03 equiv, 1.6 mmol), NaOtBu (4.97 g, 1.0 equiv, 51.7 mmol), toluene (259 mL), 4-(4-bromophenyl)dibenzothiophene (19.21 g, 1.1 equiv, 56.9 mmol), and P^{t}Bu₃ (1.05 g, 0.1 equiv, 5.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate Compound IM-7 (17.29 g, yield 74%).

By measuring utilizing FAB-MS, a mass number of m/z=451 was observed by molecular ion peak, thereby identifying Intermediate Compound IM-7.

### 2) Synthesis of Compound C23

In an Ar atmosphere, in a 300 mL three-neck flask, Intermediate Compound IM-7 (10.00 g, 22.1 mmol), Pd(dba)₂ (0.38 g, 0.03 equiv, 0.7 mmol), NaO^{t}Bu (4.26 g, 2.0 equiv, 44.3 mmol), toluene (110 mL), 2-bromodibenzofuran (6.02 g, 1.1 equiv, 24.4 mmol), and P^{t}Bu₃ (0.45 g, 0.1 equiv, 2.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound C23 (10.94 g, yield 80%).

By measuring utilizing FAB-MS, a mass number of m/z=617 was observed by molecular ion peak, thereby identifying Compound C23.

### (13) Synthesis of Compound C50

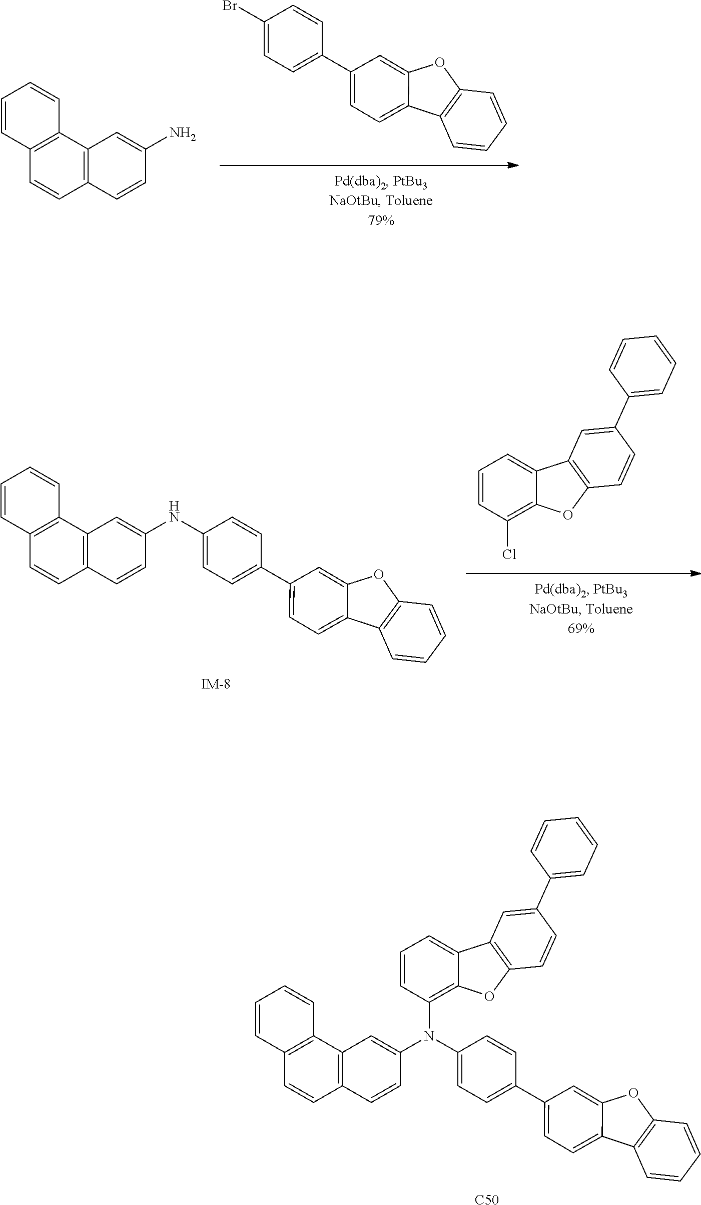

### 1) Synthesis of Intermediate Compound IM-8

In an Ar atmosphere, in a 500 mL three-neck flask, 3-aminophenanthrene (10.00 g, 51.7 mmol), Pd(dba)₂ (0.89 g, 0.03 equiv, 1.6 mmol), NaO^{t}Bu (4.97 g, 1.0 equiv, 51.7 mmol), toluene (259 mL), 3-(4-bromophenyl)dibenzofuran (18.40 g, 1.1 equiv, 56.9 mmol), and P^{t}Bu₃ (1.05 g, 0.1 equiv, 5.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate Compound IM-8 (17.80 g, yield 79%).

By measuring utilizing FAB-MS, a mass number of m/z=435 was observed by molecular ion peak, thereby identifying Intermediate Compound IM-8.

### 2) Synthesis of Compound C50

In an Ar atmosphere, in a 300 mL three-neck flask, Intermediate Compound IM-8 (10.00 g, 23.0 mmol), Pd(dba)₂ (0.40 g, 0.03 equiv, 0.7 mmol), NaOtBu (4.41 g, 2.0 equiv, 45.9 mmol), toluene (115 mL), 6-chloro-2-phenyldibenzofuran (12.09 g, 1.1 equiv, 25.3 mmol), and P^{t}Bu₃ (0.46 g, 0.1 equiv, 2.3 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound C50 (10.74 g, yield 69%).

By measuring utilizing FAB-MS, a mass number of m/z=677 was observed by molecular ion peak, thereby identifying Compound C50.

### (14) Synthesis of Compound C85

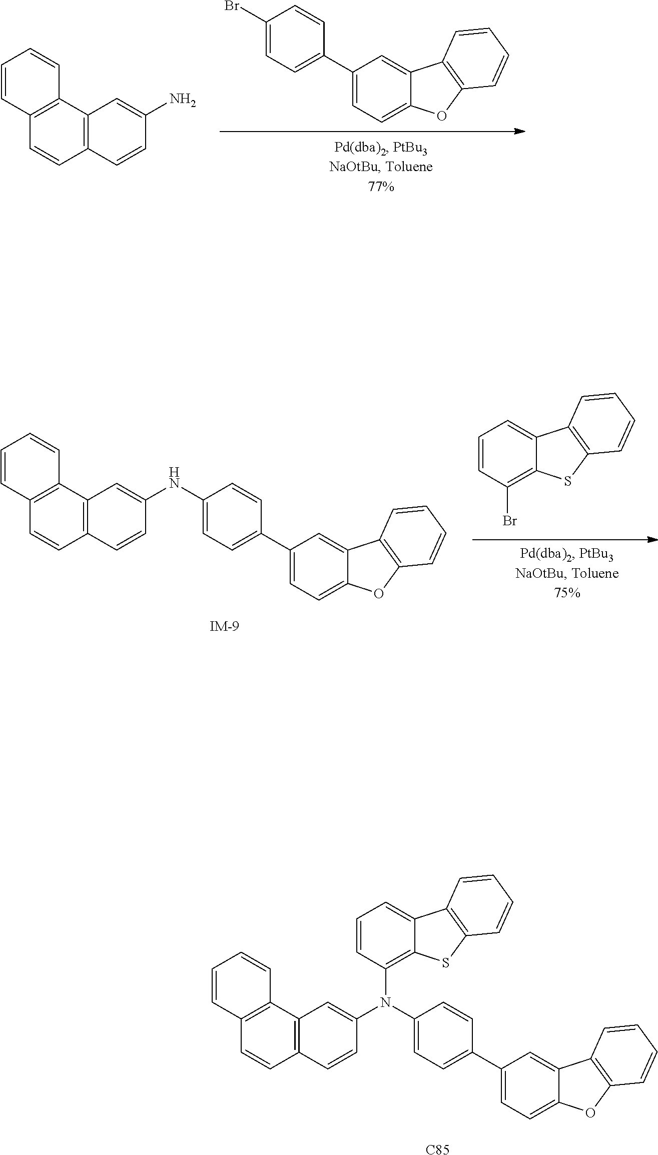

### 1) Synthesis of Intermediate Compound IM-9

In an Ar atmosphere, in a 500 mL three-neck flask, 3-aminophenanthrene (10.00 g, 51.7 mmol), Pd(dba)₂ (0.89 g, 0.03 equiv, 1.6 mmol), NaO^{t}Bu (4.97 g, 1.0 equiv, 51.7 mmol), toluene (259 mL), 2-(4-bromophenyl)dibenzofuran (18.40 g, 1.1 equiv, 56.9 mmol), and P^{t}Bu₃ (1.05 g, 0.1 equiv, 5.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate Compound IM-9 (17.35 g, yield 77%).

By measuring utilizing FAB-MS, a mass number of m/z=435 was observed by molecular ion peak, thereby identifying Intermediate Compound IM-9.

### 2) Synthesis of Compound C85

In an Ar atmosphere, in a 300 mL three-neck flask, Intermediate Compound IM-9 (10.00 g, 23.0 mmol), Pd(dba)₂ (0.40 g, 0.03 equiv, 0.7 mmol), NaOtBu (4.41 g, 2.0 equiv, 45.9 mmol), toluene (115 mL), 4-bromodibenzothiophene (6.65 g, 1.1 equiv, 25.3 mmol), and P^{t}Bu₃ (0.46 g, 0.1 equiv, 2.3 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound C85 (10.64 g, yield 75%).

By measuring utilizing FAB-MS, a mass number of m/z=617 was observed by molecular ion peak, thereby identifying Compound C85.

### (15) Synthesis of Compound C133

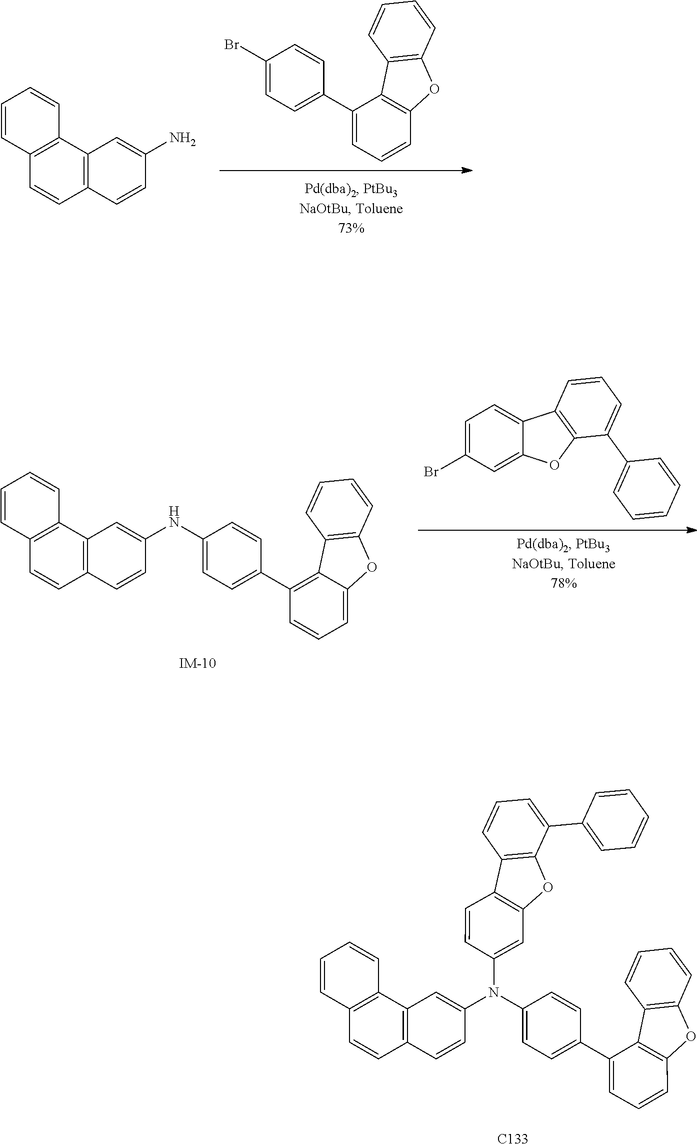

### 1) Synthesis of Intermediate Compound IM-10

In an Ar atmosphere, in a 500 mL three-neck flask, 3-aminophenanthrene (10.00 g, 51.7 mmol), Pd(dba)₂ (0.89 g, 0.03 equiv, 1.6 mmol), NaOtBu (4.97 g, 1.0 equiv, 51.7 mmol), toluene (259 mL), 1-(4-bromophenyl)dibenzofuran (18.40 g, 1.1 equiv, 56.9 mmol), and P^{t}Bu₃ (1.05 g, 0.1 equiv, 5.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate Compound IM-10 (16.45 g, yield 73%).

By measuring utilizing FAB-MS, a mass number of m/z=435 was observed by molecular ion peak, thereby identifying Intermediate Compound IM-10.

### 2) Synthesis of Compound C133

In an Ar atmosphere, in a 300 mL three-neck flask, Intermediate Compound IM-10 (10.00 g, 23.0 mmol), Pd(dba)₂ (0.40 g, 0.03 equiv, 0.7 mmol), NaOtBu (4.41 g, 2.0 equiv, 45.9 mmol), toluene (115 mL), 3-bromo-6-phenyldibenzofuran (8.16 g, 1.1 equiv, 25.3 mmol), and P^{t}Bu₃ (0.46 g, 0.1 equiv, 2.3 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound C133 (12.14 g, yield 78%).

By measuring utilizing FAB-MS, a mass number of m/z=677 was observed by molecular ion peak, thereby identifying Compound C133.

### (16) Synthesis of Compound D2

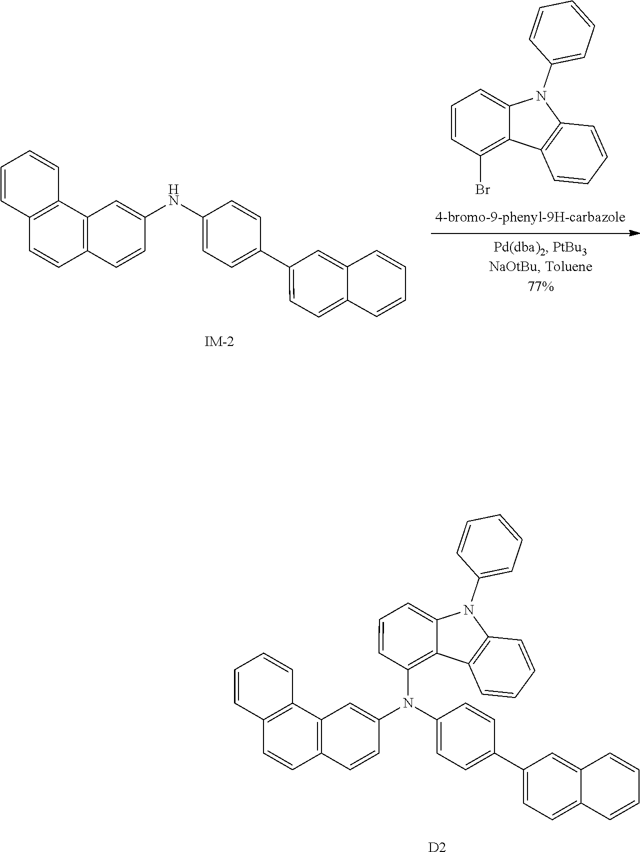

### 1) Synthesis of Compound D2

In an Ar atmosphere, in a 300 mL three-neck flask, Intermediate Compound IM-2 (10.00 g, 25.3 mmol), Pd(dba)₂ (0.44 g, 0.03 equiv, 0.8 mmol), NaO^{t}Bu (4.86 g, 2.0 equiv, 50.6 mmol), toluene (126 mL), 4-bromo-9-phenyl-9H-carbazole (8.96 g, 1.1 equiv, 27.8 mmol), and P^{t}Bu₃ (0.51 g, 0.1 equiv, 2.5 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound D2 (12.40 g, yield 77%).

By measuring utilizing FAB-MS, a mass number of m/z=636 was observed by molecular ion peak, thereby identifying Compound D2.

### (17) Synthesis of Compound D26

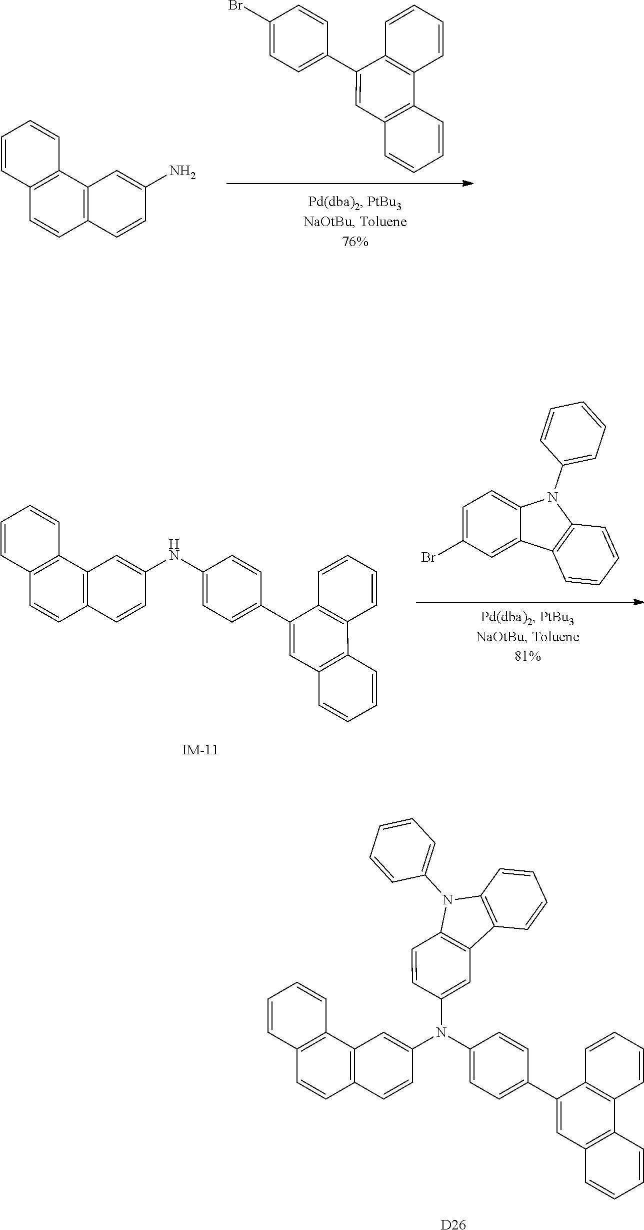

### 1) Synthesis of Intermediate Compound IM-11

In an Ar atmosphere, in a 500 mL three-neck flask, 3-aminophenanthrene (10.00 g, 51.7 mmol), Pd(dba)₂ (0.89 g, 0.03 equiv, 1.6 mmol), NaOtBu (4.97 g, 1.0 equiv, 51.7 mmol), toluene (259 mL), 9-(4-bromophenyl)phenanthrene (18.97 g, 1.1 equiv, 56.9 mmol), and P^{t}Bu₃ (1.05 g, 0.1 equiv, 5.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate Compound IM-11 (17.52 g, yield 76%).

By measuring utilizing FAB-MS, a mass number of m/z=445 was observed by molecular ion peak, thereby identifying Intermediate Compound IM-11.

### 2) Synthesis of Compound D26

In an Ar atmosphere, in a 300 mL three-neck flask, Intermediate Compound IM-11 (10.00 g, 22.4 mmol), Pd(dba)₂ (0.39 g, 0.03 equiv, 0.7 mmol), NaOtBu (4.31 g, 2.0 equiv, 44.9 mmol), toluene (112 mL), 3-bromo-9-phenyl-9H-carbazole (7.95 g, 1.1 equiv, 24.7 mmol), and P^{t}Bu₃ (0.45 g, 0.1 equiv, 2.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound D26 (12.49 g, yield 81%).

By measuring utilizing FAB-MS, a mass number of m/z=686 was observed by molecular ion peak, thereby identifying Compound D26.

### (18) Synthesis of Compound D41

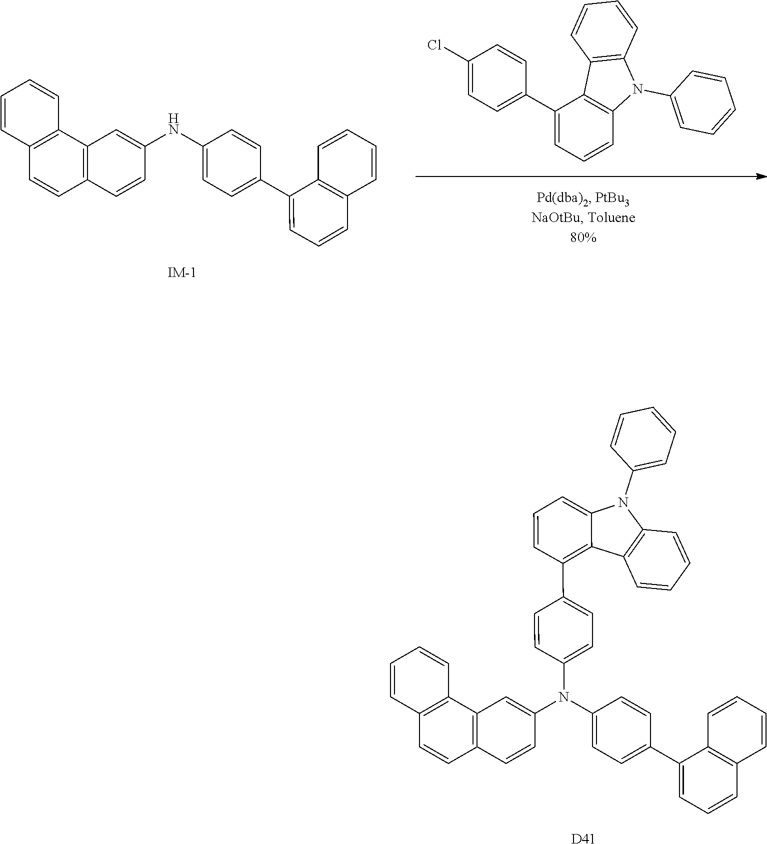

### 1) Synthesis of Compound D41

In an Ar atmosphere, in a 300 mL three-neck flask, Intermediate Compound IM-1 (10.00 g, 25.3 mmol), Pd(dba)₂ (0.44 g, 0.03 equiv, 0.8 mmol), NaO^{t}Bu (4.86 g, 2.0 equiv, 50.6 mmol), toluene (126 mL), 4-(4-chlorophenyl)-9-phenyl-9H-carbazole (9.84 g, 1.1 equiv, 27.8 mmol), and P^{t}Bu₃ (0.51 g, 0.1 equiv, 2.5 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound D41 (14.42 g, yield 80%).

By measuring utilizing FAB-MS, a mass number of m/z=712 was observed by molecular ion peak, thereby identifying Compound D41.

### (19) Synthesis of Compound D101

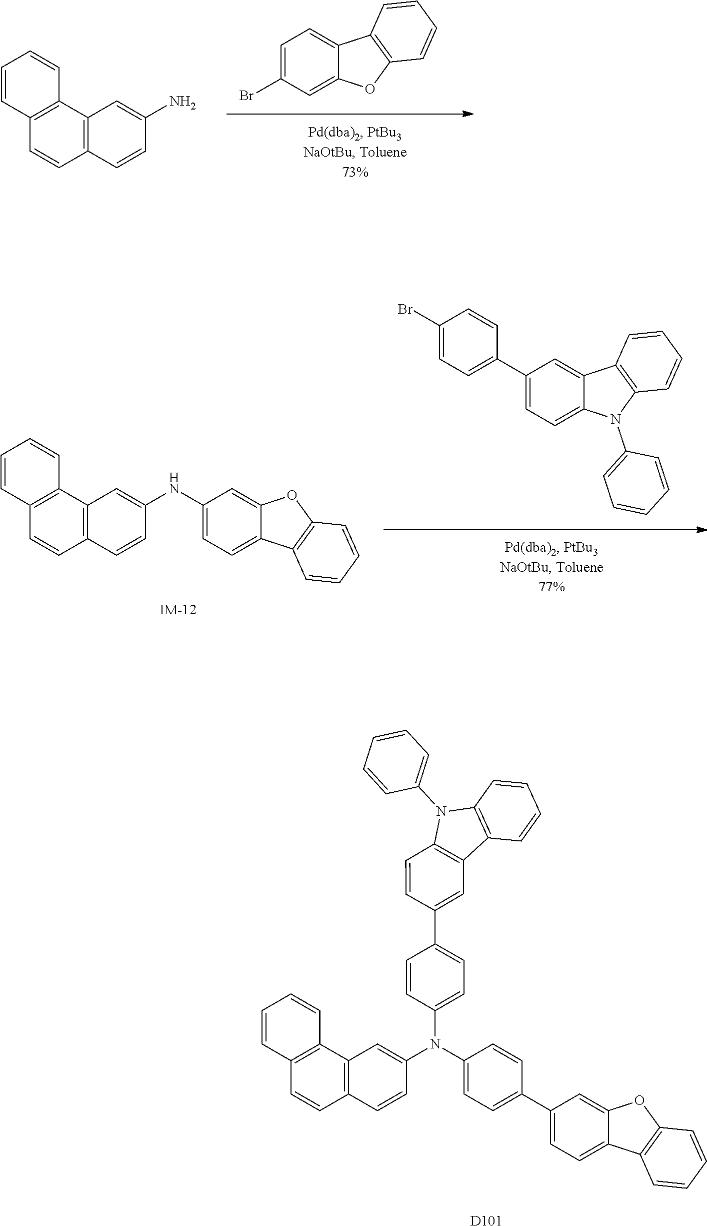

### 1) Synthesis of Intermediate Compound IM-12

In an Ar atmosphere, in a 500 mL three-neck flask, 3-aminophenanthrene (10.00 g, 51.7 mmol), Pd(dba)₂ (0.89 g, 0.03 equiv, 1.6 mmol), NaOtBu (4.97 g, 1.0 equiv, 51.7 mmol), toluene (259 mL), 3-bromodibenzofuran (14.06 g, 1.1 equiv, 56.9 mmol), and P^{t}Bu₃ (1.05 g, 0.1 equiv, 5.2 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate Compound IM-12 (13.58 g, yield 73%).

By measuring utilizing FAB-MS, a mass number of m/z=359 was observed by molecular ion peak, thereby identifying Intermediate Compound IM-12.

### 2) Synthesis of Compound D101

In an Ar atmosphere, in a 300 mL three-neck flask, Intermediate Compound IM-12 (10.00 g, 27.8 mmol), Pd(dba)₂ (0.48 g, 0.03 equiv, 0.8 mmol), NaOtBu (5.35 g, 2.0 equiv, 55.6 mmol), toluene (140 mL), 3-(4-bromophenyl)-9-phenyl-9H-carbazole (12.19 g, 1.1 equiv, 30.6 mmol), and P^{t}Bu₃ (0.56 g, 0.1 equiv, 2.8 mmol) were sequentially added, and then heated and stirred under reflux. After the resultant reaction solution was air-cooled to room temperature, organic layers were separated and obtained by adding water to the resultant reaction solution. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound D101 (14.50 g, yield 77%).

By measuring utilizing FAB-MS, a mass number of m/z=676 was observed by molecular ion peak, thereby identifying Compound D101.

### 2. Manufacture and Evaluation of Light Emitting Element

Evaluation of the light emitting elements including compounds of Examples and Comparative Examples in a hole transport layer was performed as follows. The method for manufacturing the light emitting element for the evaluation of the element is described below.

### (1) Manufacture of Light Emitting Element 1

A 1500 Å-thick ITO was patterned on a glass substrate, and then the glass substrate was washed with ultrapure water and treated with UV and ozone for about 10 minutes to form a first electrode. Thereafter, 2-TNATA was deposited to form a 600 Å-thick hole injection layer. Then, an Example Compound or a Comparative Example Compound was deposited to form a 300 Å-thick hole transport layer.

Thereafter, TBP was doped to ADN by 3% to form a 250 Å-thick emission layer. Then, Alq₃ was deposited to form a 250 Å-thick electron transport layer, and LiF was deposited to form a 10 Å-thick electron injection layer.

Then, aluminum (Al) was provided to form a 1,000 Å-thick second electrode.

In the Examples, the hole injection layer, the hole transport layer, the emission layer, the electron transport layer, the electron injection layer, and the second electrode were formed by using a vacuum deposition apparatus.

### (2) Manufacture of Light Emitting Element 2

A 1500 Å-thick ITO was patterned on a glass substrate, and then the glass substrate was washed with ultrapure water and treated with UV and ozone for about 10 minutes to form a first electrode. Thereafter, 2-TNATA was deposited to form a 600 Å-thick hole injection layer. Then, H-1-1 was deposited to form a 200 Å-thick hole transport layer, and then an Example Compound or a Comparative Example Compound was deposited to form 100 Å-thick electron blocking layer.

Thereafter, TBP was doped to ADN by 3% to form a 250 Å-thick emission layer. Then, Alq₃ was deposited to form a 250 Å-thick electron transport layer, and LiF was deposited to form a 10 Å-thick electron injection layer.

Then, aluminum (Al) was provided to form a 1,000 Å-thick second electrode.

In the Examples, the hole injection layer, the hole transport layer, the electron blocking layer, the emission layer, the electron transport layer, the electron injection layer, and the second electrode were formed by using a vacuum deposition apparatus.

For the molecular weight of Example Compound, FAB-MS was performed by using a JMS-700V manufactured by JEOL, Ltd. In addition, for the nuclear magnetic resonance (NMR) spectroscopic analysis of Example Compound, proton nuclear magnetic resonance (1H-NMR) spectroscopy was performed by using an AVAVCE300M manufactured by Bruker Biospin K.K. In the following evaluation of the light emitting elements, current densities, voltages and luminous efficiencies of the elements were measured in a dark room by using a 2400 Series Source Meter manufactured by Keithley Instruments, Inc., a CS-200, Color and Luminance Meter manufactured by Konica Minolta, Inc., and PC Program LabVIEW 8.2 for the measurement manufactured by Japan National Instrument, Inc.

Example Compounds and Comparative Example Compounds used to manufacture light emitting element 1 and light emitting element 2 are as follows:

### Example Compounds

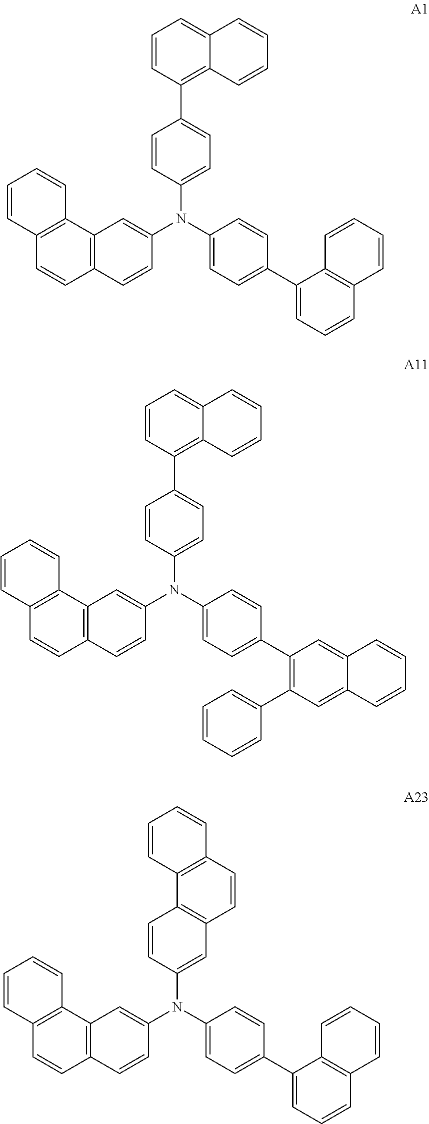 [SMILES: c1ccc2c(-c3ccc(N(c4ccc(-c5cccc6ccccc56)cc4)c4ccc5ccc6ccccc6c5c4)cc3)cccc2c1 ; c1ccc(-c2cc3ccccc3cc2-c2ccc(N(c3ccc(-c4cccc5ccccc45)cc3)c3ccc4ccc5ccccc5c4c3)cc2)cc1 ; c1ccc2c(-c3ccc(N(c4ccc5c(ccc6ccccc65)c4)c4ccc5ccc6ccccc6c5c4)cc3)cccc2c1]

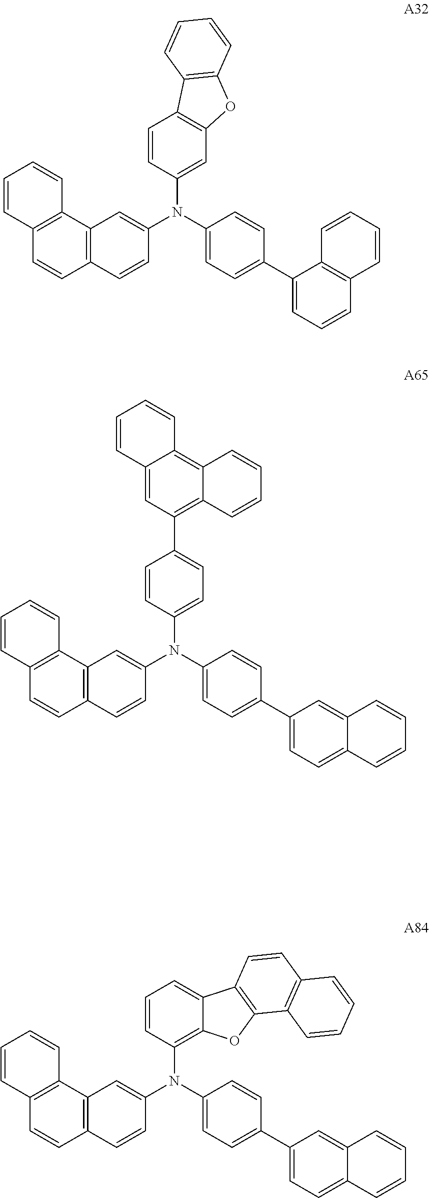 [SMILES: c1ccc2c(-c3ccc(N(c4ccc5c(c4)oc4ccccc45)c4ccc5ccc6ccccc6c5c4)cc3)cccc2c1 ; c1ccc2cc(-c3ccc(N(c4ccc(-c5cc6ccccc6c6ccccc56)cc4)c4ccc5ccc6ccccc6c5c4)cc3)ccc2c1 ; c1ccc2cc(-c3ccc(N(c4ccc5ccc6ccccc6c5c4)c4cccc5c4oc4c6ccccc6ccc54)cc3)ccc2c1]

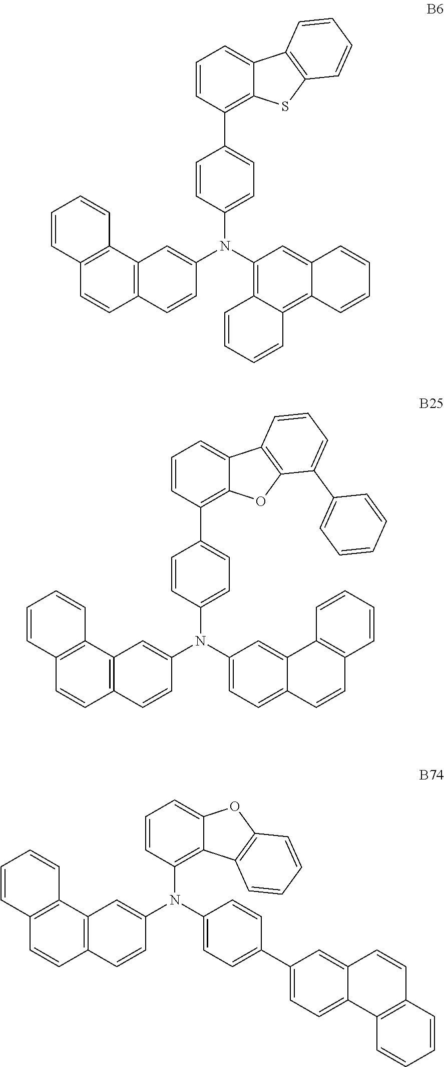 [SMILES: c1ccc2c(c1)ccc1ccc(N(c3ccc(-c4cccc5c4sc4ccccc45)cc3)c3cc4ccccc4c4ccccc34)cc12 ; c1ccc(-c2cccc3c2oc2c(-c4ccc(N(c5ccc6ccc7ccccc7c6c5)c5ccc6ccc7ccccc7c6c5)cc4)cccc23)cc1 ; c1ccc2c(c1)ccc1cc(-c3ccc(N(c4ccc5ccc6ccccc6c5c4)c4cccc5oc6ccccc6c45)cc3)ccc12]

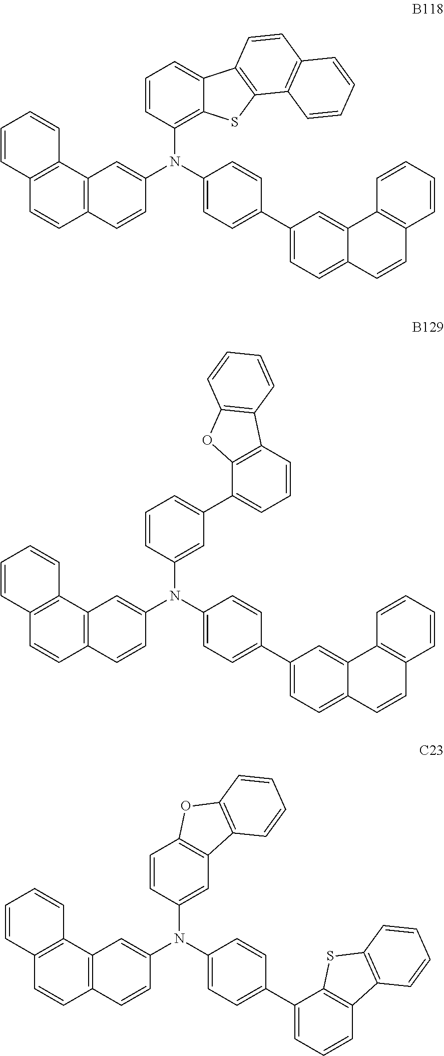 [SMILES: c1ccc2c(c1)ccc1ccc(-c3ccc(N(c4ccc5ccc6ccccc6c5c4)c4cccc5c4sc4c6ccccc6ccc54)cc3)cc12 ; c1cc(-c2cccc3c2oc2ccccc23)cc(N(c2ccc(-c3ccc4ccc5ccccc5c4c3)cc2)c2ccc3ccc4ccccc4c3c2)c1 ; c1ccc2c(c1)ccc1ccc(N(c3ccc(-c4cccc5c4sc4ccccc45)cc3)c3ccc4oc5ccccc5c4c3)cc12]

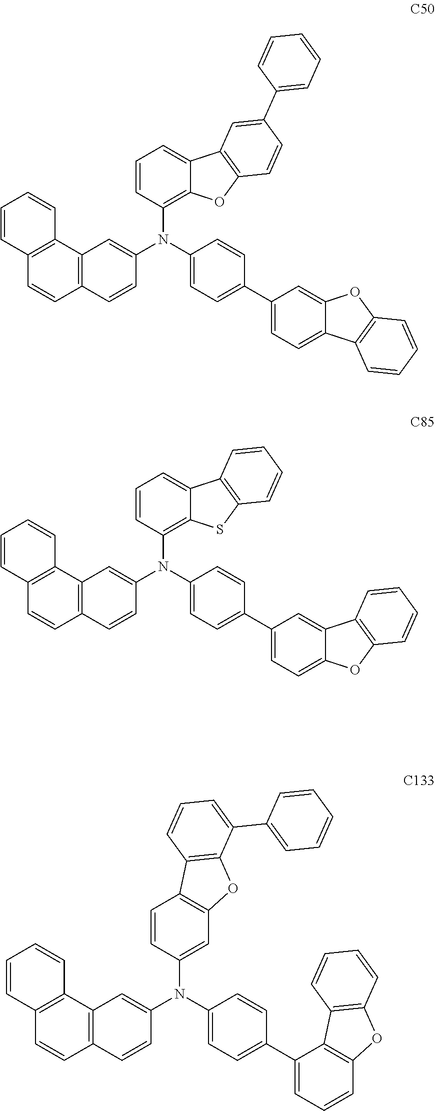 [SMILES: c1ccc(-c2ccc3oc4c(N(c5ccc(-c6ccc7c(c6)oc6ccccc67)cc5)c5ccc6ccc7ccccc7c6c5)cccc4c3c2)cc1 ; c1ccc2c(c1)ccc1ccc(N(c3ccc(-c4ccc5oc6ccccc6c5c4)cc3)c3cccc4c3sc3ccccc34)cc12 ; c1ccc(-c2cccc3c2oc2cc(N(c4ccc(-c5cccc6oc7ccccc7c56)cc4)c4ccc5ccc6ccccc6c5c4)ccc23)cc1]

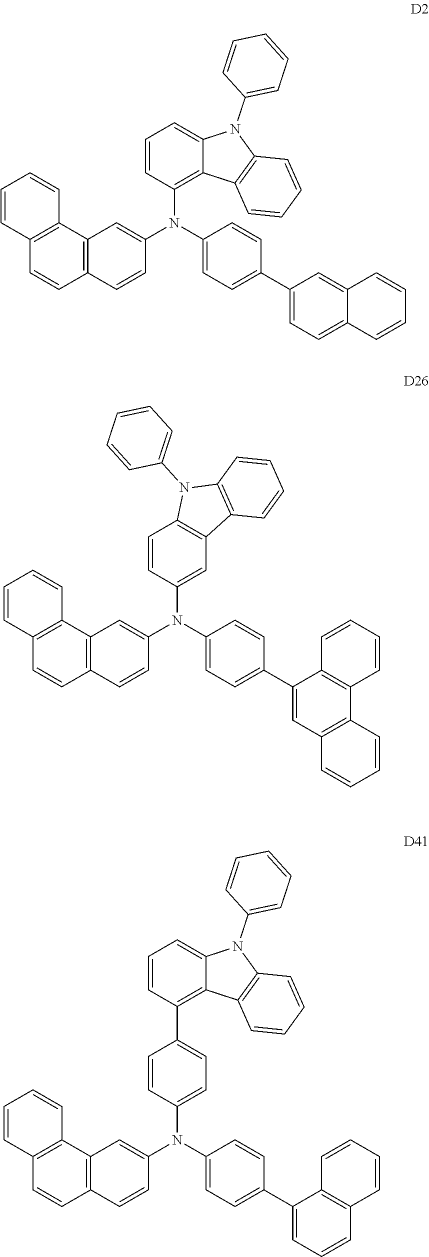 [SMILES: c1ccc(-n2c3ccccc3c3c(N(c4ccc(-c5ccc6ccccc6c5)cc4)c4ccc5ccc6ccccc6c5c4)cccc32)cc1 ; c1ccc(-n2c3ccccc3c3cc(N(c4ccc(-c5cc6ccccc6c6ccccc56)cc4)c4ccc5ccc6ccccc6c5c4)ccc32)cc1 ; c1ccc(-n2c3ccccc3c3c(-c4ccc(N(c5ccc(-c6cccc7ccccc67)cc5)c5ccc6ccc7ccccc7c6c5)cc4)cccc32)cc1]

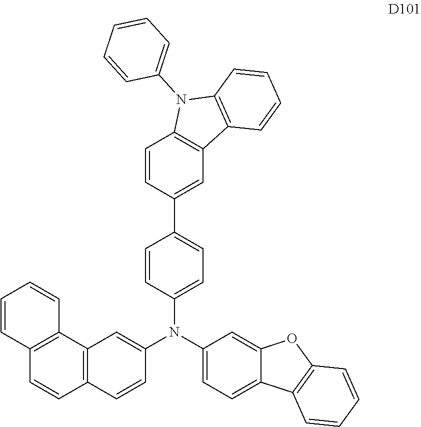 [SMILES: c1ccc(-n2c3ccccc3c3cc(-c4ccc(N(c5ccc6c(c5)oc5ccccc56)c5ccc6ccc7ccccc7c6c5)cc4)ccc32)cc1]

### Comparative Example Compounds

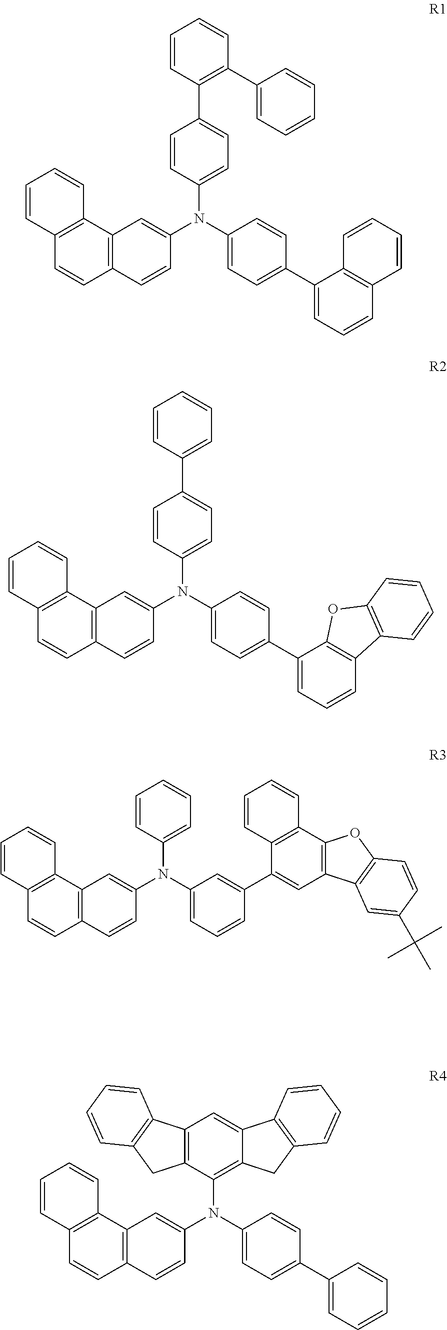 [SMILES: c1ccc(-c2ccccc2-c2ccc(N(c3ccc(-c4cccc5ccccc45)cc3)c3ccc4ccc5ccccc5c4c3)cc2)cc1 ; c1ccc(-c2ccc(N(c3ccc(-c4cccc5c4oc4ccccc45)cc3)c3ccc4ccc5ccccc5c4c3)cc2)cc1 ; CC(C)(C)c1ccc2oc3c4ccccc4c(-c4cccc(N(c5ccccc5)c5ccc6ccc7ccccc7c6c5)c4)cc3c2c1 ; c1ccc(-c2ccc(N(c3ccc4ccc5ccccc5c4c3)c3c4c(cc5c3Cc3ccccc3-5)-c3ccccc3C4)cc2)cc1]

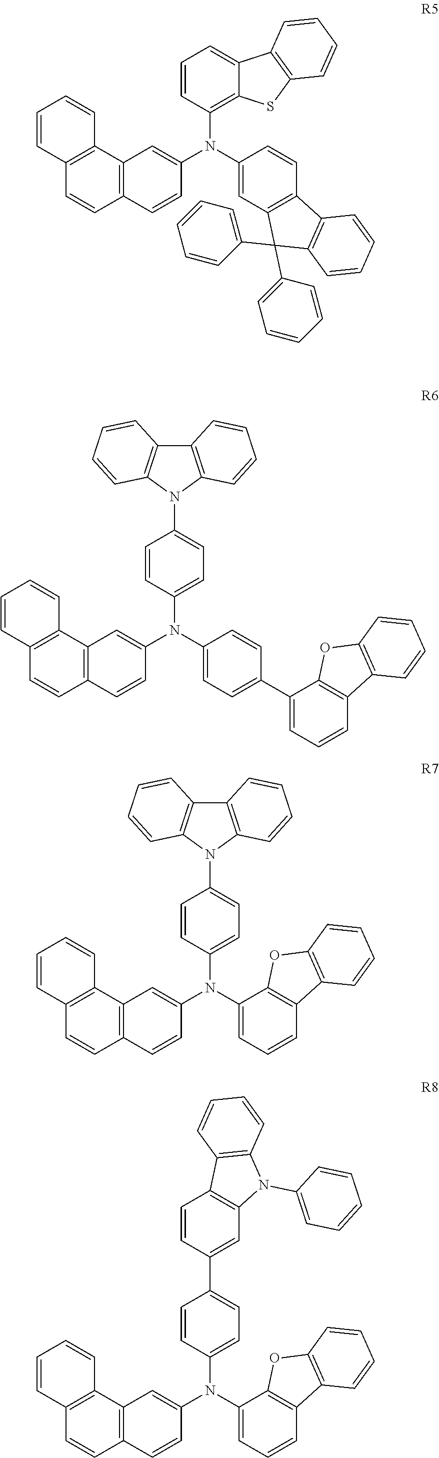 [SMILES: c1ccc(C2(c3ccccc3)c3ccccc3-c3ccc(N(c4ccc5ccc6ccccc6c5c4)c4cccc5c4sc4ccccc45)cc32)cc1 ; c1ccc2c(c1)ccc1ccc(N(c3ccc(-c4cccc5c4oc4ccccc45)cc3)c3ccc(-n4c5ccccc5c5ccccc54)cc3)cc12 ; c1ccc2c(c1)ccc1ccc(N(c3ccc(-n4c5ccccc5c5ccccc54)cc3)c3cccc4c3oc3ccccc34)cc12 ; c1ccc(-n2c3ccccc3c3ccc(-c4ccc(N(c5ccc6ccc7ccccc7c6c5)c5cccc6c5oc5ccccc56)cc4)cc32)cc1]

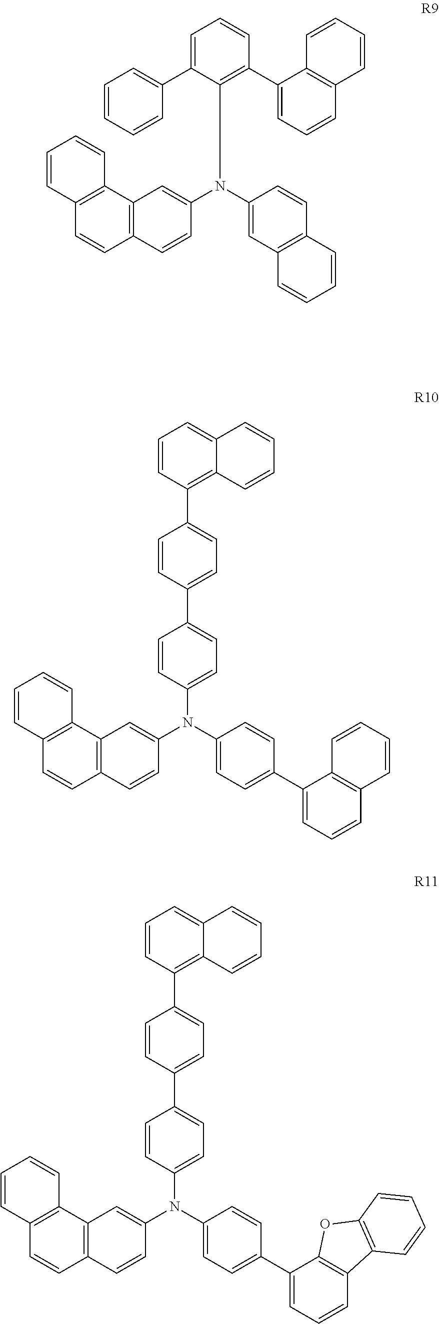 [SMILES: c1ccc(-c2cccc(-c3cccc4ccccc34)c2N(c2ccc3ccccc3c2)c2ccc3ccc4ccccc4c3c2)cc1 ; c1ccc2c(-c3ccc(-c4ccc(N(c5ccc(-c6cccc7ccccc67)cc5)c5ccc6ccc7ccccc7c6c5)cc4)cc3)cccc2c1 ; c1ccc2c(-c3ccc(-c4ccc(N(c5ccc(-c6cccc7c6oc6ccccc67)cc5)c5ccc6ccc7ccccc7c6c5)cc4)cc3)cccc2c1]

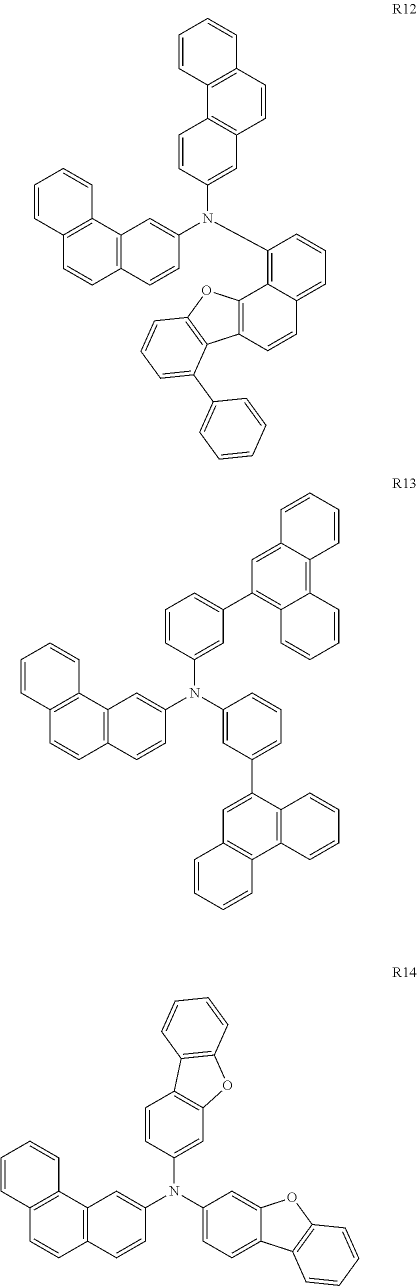 [SMILES: c1ccc(-c2cccc3oc4c(ccc5cccc(N(c6ccc7c(ccc8ccccc87)c6)c6ccc7ccc8ccccc8c7c6)c54)c23)cc1 ; c1cc(-c2cc3ccccc3c3ccccc23)cc(N(c2cccc(-c3cc4ccccc4c4ccccc34)c2)c2ccc3ccc4ccccc4c3c2)c1 ; c1ccc2c(c1)ccc1ccc(N(c3ccc4c(c3)oc3ccccc34)c3ccc4c(c3)oc3ccccc34)cc12]

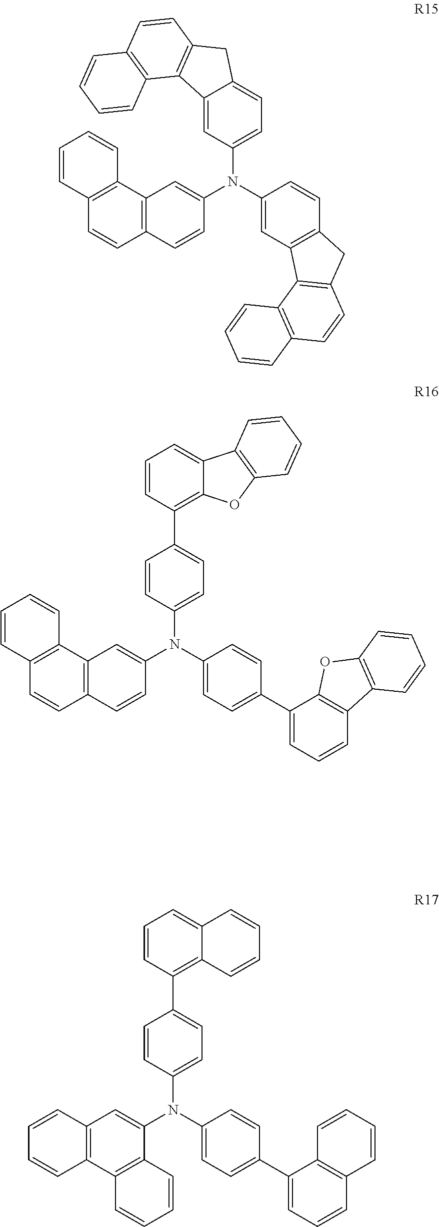 [SMILES: c1ccc2c3c(ccc2c1)Cc1ccc(N(c2ccc4c(c2)-c2c(ccc5ccccc25)C4)c2ccc4ccc5ccccc5c4c2)cc1-3 ; c1ccc2c(c1)ccc1ccc(N(c3ccc(-c4cccc5c4oc4ccccc45)cc3)c3ccc(-c4cccc5c4oc4ccccc45)cc3)cc12 ; c1ccc2c(-c3ccc(N(c4ccc(-c5cccc6ccccc56)cc4)c4cc5ccccc5c5ccccc45)cc3)cccc2c1]

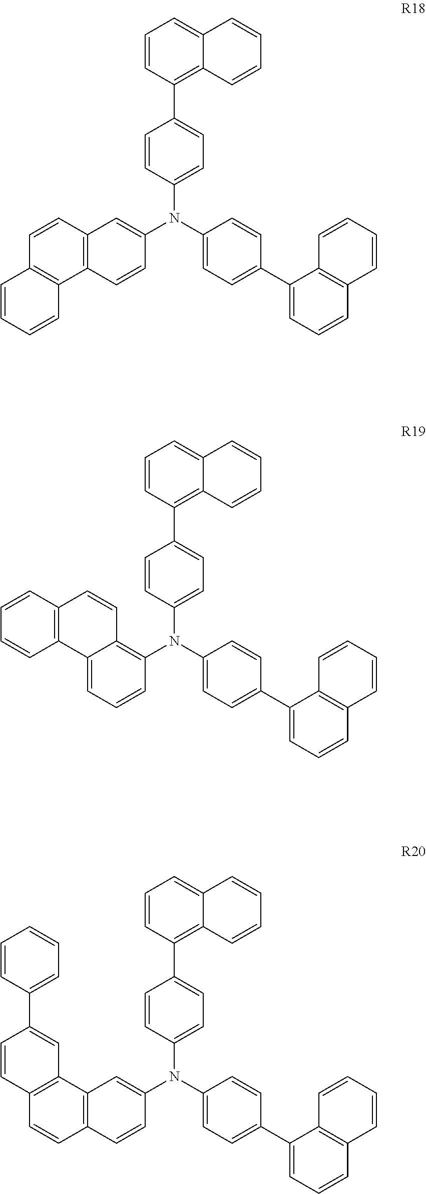 [SMILES: c1ccc2c(-c3ccc(N(c4ccc(-c5cccc6ccccc56)cc4)c4ccc5c(ccc6ccccc65)c4)cc3)cccc2c1 ; c1ccc2c(-c3ccc(N(c4ccc(-c5cccc6ccccc56)cc4)c4cccc5c4ccc4ccccc45)cc3)cccc2c1 ; c1ccc(-c2ccc3ccc4ccc(N(c5ccc(-c6cccc7ccccc67)cc5)c5ccc(-c6cccc7ccccc67)cc5)cc4c3c2)cc1]

In addition, compounds of each functional layer used to manufacture light emitting elements 1 and 2 are as follows:

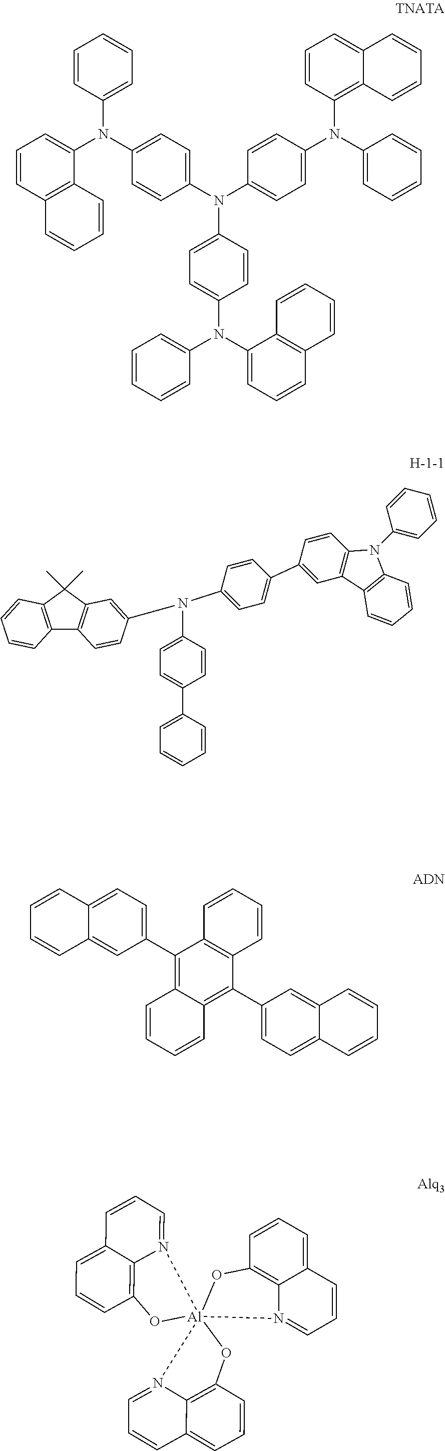

### (3) Evaluation of Light Emitting Element 1 and Light Emitting Element 2

The evaluation results of light emitting element 1 with respect to Examples 1-1 to 1-19 and Comparative Examples 1-1 to 1-20 are listed in Table 1, and the evaluation results of light emitting element 2 with respect to Examples 2-1 to 2-19 and Comparative Examples 2-1 to 2-20 are listed in Table 2. The maximum luminous efficiencies and half service lives of light emitting element 1 and light emitting element 2 are listed in comparison in each of Tables 1 and 2. In the evaluation results of the characteristics for Examples and Comparative Examples listed in Tables 1 and 2, the luminous efficiency shows the efficiency value at a current density of 10 mA/cm².

The element service life is a relative value showing a time when the brightness value is 50% of an initial brightness during the continuous operation of the element at 1,000 cd/m² compared with Comparative Examples 1-1 and 2-1.

The luminous efficiencies and element service lives in Tables 1 and 2 below represent compared values when it is assumed that each of the luminous efficiency and service life of Comparative Examples 1-1 and 2-1, respectively, is 100%.

TABLE 1

| Examples of | | Luminous | Element |
|---|---|---|---|
| manufactured | Hole transport layer | efficiency | service life |
| elements | material | @10 mA/cm² | LT50 |
| Example 1-1 | Example Compound | 151% | 177% |
| | A1 | | |
| Example 1-2 | Example Compound | 147% | 185% |
| | A11 | | |
| Example 1-3 | Example Compound | 145% | 183% |
| | A23 | | |
| Example 1-4 | Example Compound | 145% | 190% |
| | A32 | | |
| Example 1-5 | Example Compound | 153% | 175% |
| | A65 | | |
| Example 1-6 | Example Compound | 152% | 193% |
| | A84 | | |
| Example 1-7 | Example Compound | 155% | 179% |
| | B6 | | |
| Example 1-8 | Example Compound | 147% | 187% |
| | B25 | | |
| Example 1-9 | Example Compound | 154% | 173% |
| | B74 | | |
| Example 1-10 | Example Compound | 151% | 190% |
| | B118 | | |
| Example 1-11 | Example Compound | 155% | 173% |
| | B129 | | |
| Example 1-12 | Example Compound | 145% | 178% |
| | C23 | | |
| Example 1-13 | Example Compound | 153% | 184% |
| | C50 | | |
| Example 1-14 | Example Compound | 150% | 189% |
| | C85 | | |
| Example 1-15 | Example Compound | 151% | 181% |
| | C133 | | |
| Example 1-16 | Example Compound | 155% | 179% |
| | D2 | | |
| Example 1-17 | Example Compound | 152% | 183% |
| | D26 | | |
| Example 1-18 | Example Compound | 153% | 174% |
| | D41 | | |
| Example 1-19 | Example Compound | 150% | 187% |
| | D101 | | |
| Comparative | Comparative Example | 100% | 100% |
| Example 1-1 | Compound R1 | | |
| Comparative | Comparative Example | 101% | 98% |
| Example 1-2 | Compound R2 | | |
| Comparative | Comparative Example | 96% | 87% |
| Example 1-3 | Compound R3 | | |
| Comparative | Comparative Example | 99% | 95% |
| Example 1-4 | Compound R4 | | |
| Comparative | Comparative Example | 105% | 105% |
| Example 1-5 | Compound R5 | | |
| Comparative | Comparative Example | 107% | 94% |
| Example 1-6 | Compound R6 | | |
| Comparative | Comparative Example | 103% | 89% |
| Example 1-7 | Compound R7 | | |
| Comparative | Comparative Example | 103% | 88% |
| Example 1-8 | Compound R8 | | |
| Comparative | Comparative Example | 106% | 79% |
| Example 1-9 | Compound R9 | | |
| Comparative | Comparative Example | 110% | 121% |
| Example 1-10 | Compound R10 | | |
| Comparative | Comparative Example | 108% | 115% |
| Example 1-11 | Compound R11 | | |
| Comparative | Comparative Example | 99% | 105% |
| Example 1-12 | Compound R12 | | |
| Comparative | Comparative Example | 104% | 85% |
| Example 1-13 | Compound R13 | | |
| Comparative | Comparative Example | 100% | 120% |
| Example 1-14 | Compound R14 | | |
| Comparative | Comparative Example | 107% | 110% |
| Example 1-15 | Compound R15 | | |
| Comparative | Comparative Example | 120% | 125% |
| Example 1-16 | Compound R16 | | |
| Comparative | Comparative Example | 122% | 109% |
| Example 1-17 | Compound R17 | | |
| Comparative | Comparative Example | 99% | 121% |
| Example 1-18 | Compound R18 | | |
| Comparative | Comparative Example | 125% | 98% |
| Example 1-19 | Compound R19 | | |
| Comparative | Comparative Example | 110% | 113% |
| Example 1-20 | Compound R20 | | |

Referring to the results of Table 1, it can be seen that Examples of the light emitting elements using the amine compounds of according to embodiments of the present disclosure as a hole transport layer material exhibit excellent luminous efficiency and long service life characteristics.

Although the present disclosure is not limited by any particular mechanism or theory, the above results are further explained as follows. The amine compounds of examples, in which the third position of the phenanthrene moiety is directly linked to the nitrogen atom of the amine, may have improved hole transport property because the HOMO orbital expands to thus contribute to the improvement in stability of the radical or radical cation state and the phenanthrene group having high planarity makes the intermolecular interaction effective. In addition, the amine compounds of examples have the phenanthrene skeleton at the position near to the center of the molecule, thereby suppressing or reducing excessive elevation of the deposition temperature and the deterioration of the material due to the deposition process. Moreover, the amine compound of an embodiment may improve electron resistance and exciton resistance of the material by the introduction of two substituents. Therefore, the light emitting elements of Examples including the amine compounds of examples may have an improvement in luminous efficiency and service life.

The Comparative Example Compounds used in Comparative Examples 1-1 to 1-4 each have only one substituent among substituents represented by Formula 2 to Formula 5 in the amine compound represented by Formula 1 of an embodiment. Accordingly, Comparative Examples 1-1 to 1-4 have insufficient electron resistance and exciton resistance, and thus, both the luminous efficiency and the element service life are reduced compared with Examples.

The Comparative Example Compound used in Comparative Example 1-5 is a material having a fluorene group, the periphery of the sp3 carbon atom in the fluorene skeleton is unstable in the radical or radical cation state, causing the decomposition of the material, and thus, both the luminous efficiency and the element service life are reduced compared with Examples.

The Comparative Example Compounds used in Comparative Examples 1-6 and 1-7 are each an amine compound having a carbazole group, but have the linkage position different from the substituent represented by Formula 5 of the present disclosure, and the carrier balance is collapsed, and thus, both the element efficiency and the service life are reduced.

The Comparative Example Compounds used in Comparative Examples 1-8 and 1-9 are each an amine compound in which a naphthyl group is directly linked to the nitrogen atom, and both the luminous efficiency and the element service life are reduced compared with Examples. It is believed that the naphthyl group is directly linked to the nitrogen atom, and thus, the electron density of the naphthalene ring is elevated, and the naphthyl group having high reactivity by nature is further destabilized, and thus, the deterioration of material occurs during the deposition process and the driving by electrification.

The Comparative Example Compounds used in Comparative Examples 1-10 and 1-11 are amine compounds having biphenylene as a linking group between the terminal naphthalene skeleton and the nitrogen atom, and the deposition temperature of the amine compound is elevated, thereby causing the deterioration of material, and thus, both the luminous efficiency and the element service life are reduced compared with Examples.

The Comparative Example Compound used in Comparative Example 1-12 is a material in which both a 2-phenanthrene group and a benzonaphthofuran group are directly linked to the nitrogen atom, and both the luminous efficiency and the element service life are reduced compared with Examples. It is thought that this is because the bulky substituents are concentrated at the periphery of the central nitrogen atom, and thus, the amine compound contained as a material of the light emitting element becomes unstable in the radical or radical cation state, and thus is decomposed.

The Comparative Example Compound used in Comparative Example 1-13 is a material having three phenanthrene groups in the molecule, and the deposition temperature of the material is elevated, thereby causing the deterioration of material, and thus, both the luminous efficiency and the element service life are reduced compared with Examples.

The Comparative Example Compounds used in Comparative Examples 1-14 and 1-15 are materials in which two dibenzoheterole groups are directly linked to the nitrogen atom, and the Comparative Example Compound used in Comparative Example 1-16 is a material in which two dibenzoheterole groups are linked to the nitrogen atom with a linking group located therebetween. For all the Comparative Example Compounds used in Comparative Examples 1-14 to 1-16, both the luminous efficiency and the element service life are reduced compared with Examples. Referring to the evaluation results of Examples 1-12 to 1-15, the material having two dibenzoheterole groups, in which one is directly linked to the nitrogen atom and the other is linked to the nitrogen atom with a linking group located therebetween, exhibits excellent element characteristics, and from this, it is thought that the sterical and electrical effect of the dibenzoheterole ring provide beneficial effects.

It can be confirmed that the Comparative Example Compounds used in Comparative Examples 1-17 to 1-19 are materials in which the linkage position of the phenanthrene group is different, but the characteristic specificity of the 3-phenanthrene structure is lost, and thus, both the luminous efficiency and the element service life are reduced compared with Examples.

It can be confirmed that the Comparative Example Compound used in Comparative Example 1-20 is a material having a phenyl group substituted at a phenanthrene skeleton, but the deposition temperature of the material is elevated, thereby causing the deterioration of material, and thus both the luminous efficiency and the element service life are reduced compared with Examples. The above explanation is provided based on present information and belief, but the present disclosure is not limited to any particular mechanism or theory.

TABLE 2

| Examples of | | Luminous | Element |
|---|---|---|---|
| manufactured | Electron blocking | efficiency | service life |
| elements | layer material | @10 mA/cm² | LT50 |
| Example 2-1 | Example Compound | 148% | 180% |
| | A1 | | |
| Example 2-2 | Example Compound | 147% | 182% |
| | A11 | | |
| Example 2-3 | Example Compound | 143% | 186% |
| | A23 | | |
| Example 2-4 | Example Compound | 147% | 192% |
| | A32 | | |
| Example 2-5 | Example Compound | 150% | 178% |
| | A65 | | |
| Example 2-6 | Example Compound | 156% | 189% |
| | A84 | | |
| Example 2-7 | Example Compound | 158% | 182% |
| | B6 | | |
| Example 2-8 | Example Compound | 141% | 185% |
| | B25 | | |
| Example 2-9 | Example Compound | 150% | 178% |
| | B74 | | |
| Example 2-10 | Example Compound | 147% | 186% |
| | B118 | | |
| Example 2-11 | Example Compound | 152% | 178% |
| | B129 | | |
| Example 2-12 | Example Compound | 148% | 171% |
| | C23 | | |
| Example 2-13 | Example Compound | 149% | 179% |
| | C50 | | |
| Example 2-14 | Example Compound | 153% | 191% |
| | C85 | | |
| Example 2-15 | Example Compound | 155% | 179% |
| | C133 | | |
| Example 2-16 | Example Compound | 157% | 175% |
| | D2 | | |
| Example 2-17 | Example Compound | 155% | 181% |
| | D26 | | |
| Example 2-18 | Example Compound | 156% | 177% |
| | C41 | | |
| Example 2-19 | Example Compound | 150% | 179% |
| | C101 | | |
| Comparative | Comparative Example | 100% | 100% |
| Example 2-1 | Compound R1 | | |
| Comparative | Comparative Example | 99% | 102% |
| Example 2-2 | Compound R2 | | |
| Comparative | Comparative Example | 97% | 90% |
| Example 2-3 | Compound R3 | | |
| Comparative | Comparative Example | 99% | 91% |
| Example 2-4 | Compound R4 | | |
| Comparative | Comparative Example | 103% | 109% |
| Example 2-5 | Compound R5 | | |
| Comparative | Comparative Example | 105% | 101% |
| Example 2-6 | Compound R6 | | |
| Comparative | Comparative Example | 99% | 95% |
| Example 2-7 | Compound R7 | | |
| Comparative | Comparative Example | 103% | 92% |
| Example 2-8 | Compound R8 | | |
| Comparative | Comparative Example | 104% | 86% |
| Example 2-9 | Compound R9 | | |
| Comparative | Comparative Example | 108% | 111% |
| Example 2-10 | Compound R10 | | |
| Comparative | Comparative Example | 108% | 110% |
| Example 2-11 | Compound R11 | | |
| Comparative | Comparative Example | 100% | 101% |
| Example 2-12 | Compound R12 | | |
| Comparative | Comparative Example | 101% | 85% |
| Example 2-13 | Compound R13 | | |
| Comparative | Comparative Example | 99% | 112% |
| Example 2-14 | Compound R14 | | |
| Comparative | Comparative Example | 104% | 101% |
| Example 2-15 | Compound R15 | | |
| Comparative | Comparative Example | 117% | 117% |
| Example 2-16 | Compound R16 | | |
| Comparative | Comparative Example | 116% | 104% |
| Example 2-17 | Compound R17 | | |
| Comparative | Comparative Example | 99% | 101% |
| Example 2-18 | Compound R18 | | |
| Comparative | Comparative Example | 121% | 98% |
| Example 2-19 | Compound R19 | | |
| Comparative | Comparative Example | 109% | 107% |
| Example 2-20 | Compound R20 | | |

Referring to the results of Table 2, it can be confirmed that the light emitting elements of Examples 2-1 to 2-19 exhibit long service life and high efficiency characteristics compared with those of Comparative Examples 2-1 to 2-20. That is, it can be seen that even when the amine compound of an example is used in the electron blocking layer, the light emitting element may exhibit excellent device characteristics.

Thus, the compounds used in Examples may improve luminous efficiency and luminous service life at the same time compared with the compound used in Comparative Examples. That is, the amine compound, in which the phenanthrene moiety is directly linked to the nitrogen atom, and two substituents from among the naphthyl group, the phenanthryl group, the benzoheterole group, and/or the carbazole group are introduced, is used in the light emitting element according to an embodiment, and thus it is possible to improve the element efficiency and the element service life at the same time.

The light emitting element of an embodiment may include the amine compound of an embodiment, thereby exhibiting high efficiency and long service life characteristics.

The amine compound of an embodiment may be used to achieve improved characteristics of the light emitting element having high efficiency and a long service life.

Although the subject matter of the present disclosure has been described with reference to embodiments of the present disclosure, it will be understood that the present disclosure should not be limited to these embodiments but various changes and modifications can be made by those skilled in the art without departing from the spirit and scope of the present disclosure. Accordingly, the technical scope of the present disclosure is not intended to be limited to the contents set forth in the detailed description of the specification, but is intended to be defined by the appended claims, and equivalents thereof.

What is claimed is:

1. A light emitting element comprising:
a first electrode;
a second electrode on the first electrode; and
at least one functional layer which is between the first electrode and the second electrode and comprises an amine compound represented by Formula 1 below:
Formula 1 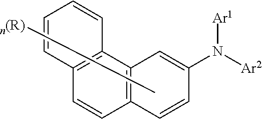
wherein, in Formula 1 above,
R is a hydrogen atom, a deuterium atom, a halogen atom, or a substituted or unsubstituted alkyl group having 1 to 20 carbon atoms,
n is an integer of 0 to 9, and
Ar¹ and Ar² are each independently represented by any one selected from among Formula 2 to Formula 5 below: 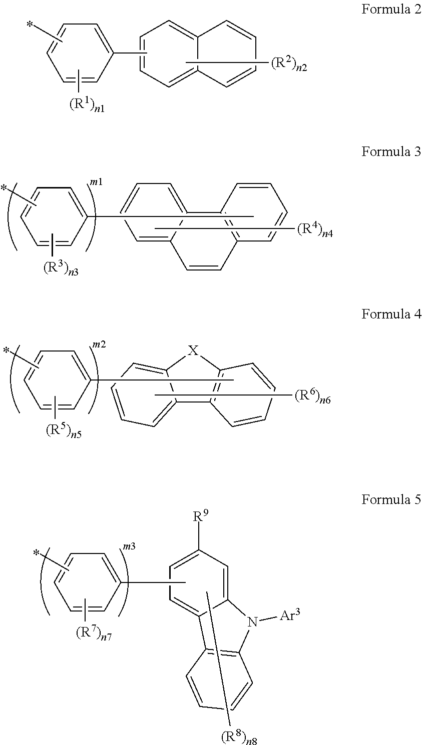
wherein, in Formula 4 above, X is O or S,
in Formula 5 above, Ar³ is a substituted or unsubstituted phenyl group, and
in Formula 2 to Formula 5 above,
R¹ to R⁵, R⁷, and R⁹ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms,
R⁶ and R⁸ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, or adjacent R⁶'s or adjacent R⁸'s are bonded to each to form an aromatic ring,
n1, n3, n5, and n7 are each independently an integer of 0 to 4, n2 and n6 are each independently an integer of 0 to 7, n4 is an integer of 0 to 9, and n8 is an integer of 0 to 6,
m1 to m3 are each independently 0 or 1,
when Ar¹ and Ar² are each independently represented by any one selected from among Formula 3 to Formula 5 above, at least one selected from among m1 to m3 is 1,
the case where both Ar¹ and Ar² are represented by Formula 3 above is excluded,
when both Ar¹ and Ar² are represented by Formula 4 above, any one selected from among two m2's is 1, and the other is 0, and
a structure in which any hydrogen atom in the molecule is substituted with a deuterium atom is included.

2. The light emitting element of claim 1, wherein the at least one functional layer comprises an emission layer, a hole transport region between the first electrode and the emission layer, and an electron transport region between the emission layer and the second electrode, and
the hole transport region comprises the amine compound.

3. The light emitting element of claim 2, wherein the hole transport region comprises at least one of a hole injection layer, a hole transport layer, or an electron blocking layer, and
at least one of the hole transport layer or the electron blocking layer comprises the amine compound.

4. The light emitting element of claim 1, wherein Formula 2 above is represented by Formula 2-1 or Formula 2-2 below: 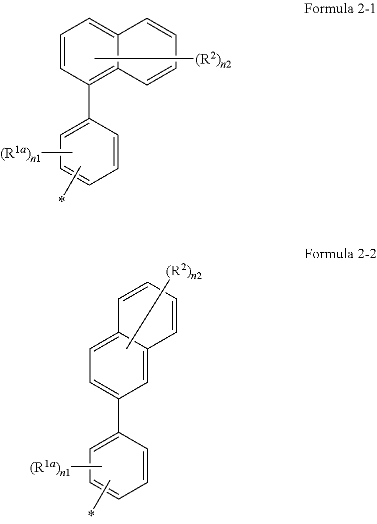
wherein, in Formula 2-1 and Formula 2-2 above,
R^{1a} is a hydrogen atom, a deuterium atom, or a substituted or unsubstituted phenyl group, and
n1, n2 and R² are the same as defined with respect to Formula 2 above.

5. The light emitting element of claim 4, wherein Formula 2-1 above is represented by 2-a or 2-b below, and Formula 2-2 above is represented by 2-c or 2-d below: 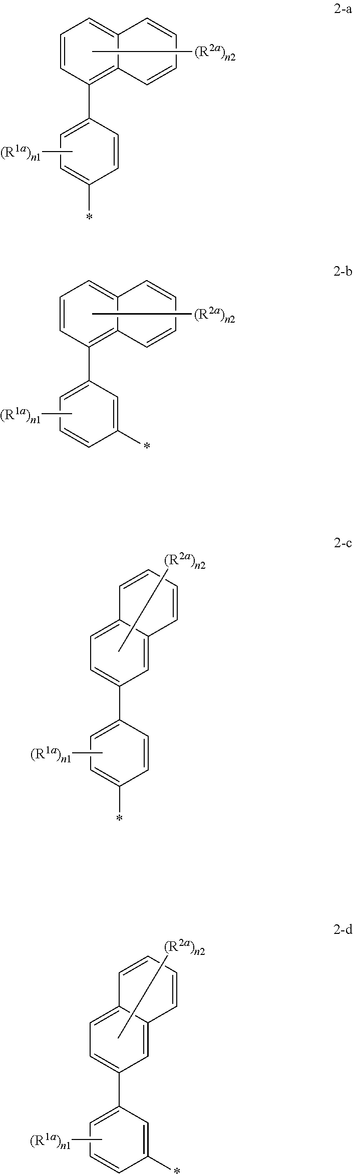
wherein, in 2-a to 2-d above,
R^{2a} is a hydrogen atom, a deuterium atom, or a substituted or unsubstituted aryl group having 6 to 15 carbon atoms, and
n1, n2, and R^{1a} are the same as defined with respect to Formula 2-1 and Formula 2-2 above.

6. The light emitting element of claim 1, wherein Formula 3 above is represented by any one selected from among Formula 3-1 to Formula 3-5 below: 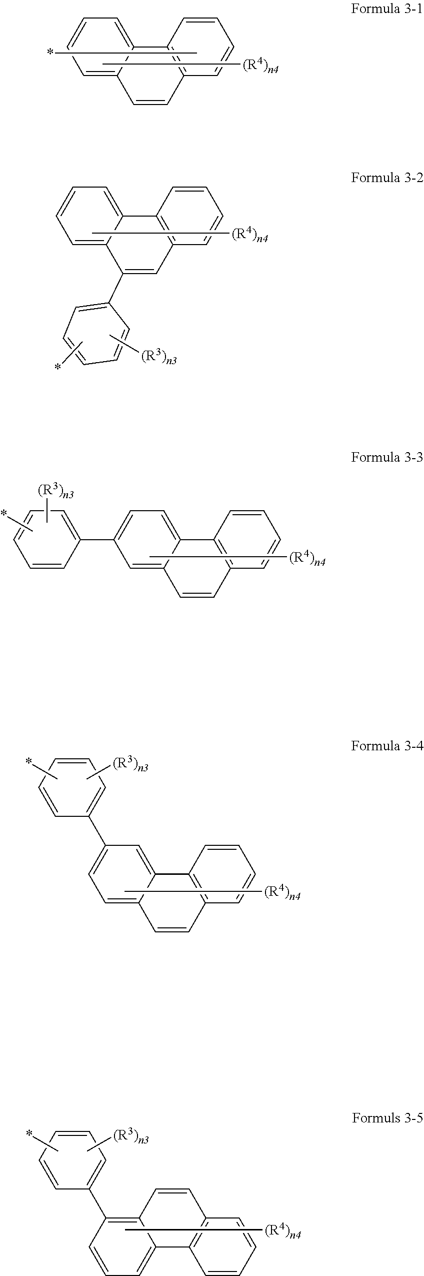
wherein, in Formula 3-1 to Formula 3-5 above,
R^{3a} is a hydrogen atom, a deuterium atom, or a substituted or unsubstituted phenyl group, and
n3, n4, and R⁴ are the same as defined with respect to Formula 3 above.

7. The light emitting element of claim 6, wherein Formula 3-1 above is represented by any one selected from among 3-a to 3-d below,
Formula 3-2 above is represented by 3-e below,
Formula 3-3 above is represented by 3-f below,
Formula 3-4 above is represented by 3-g below, and
Formula 3-5 above is represented by 3-h below: 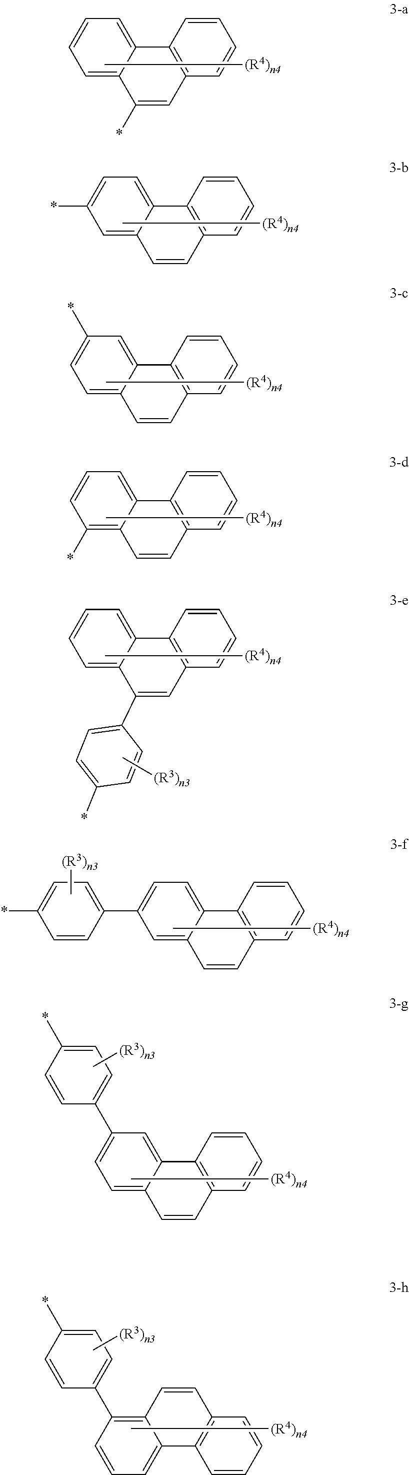
wherein, in 3-a to 3-h above,
n3, n4, and R^{3a} are the same as defined with respect to Formula 3-1 to Formula 3-5 above, and
R^{4a} is a hydrogen atom, a deuterium atom, a halogen atom, or a substituted or unsubstituted alkyl group having 1 to 10 carbon atoms.

8. The light emitting element of claim 1, wherein Formula 4 above is represented by Formula 4-1 or Formula 4-2 below: 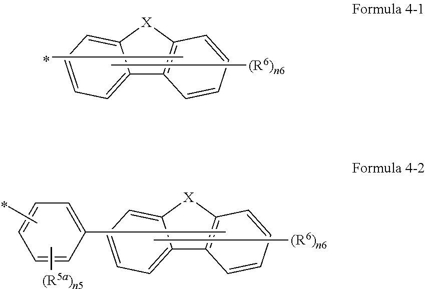
wherein, in Formula 4-1 and Formula 4-2 above,
R^{5a} is a hydrogen atom, a deuterium atom, or a substituted or unsubstituted phenyl group, and
X, n5, n6, and R⁶ are the same as defined with respect to Formula 4 above.

9. The light emitting element of claim 8, wherein Formula 4-1 above is represented by 4-a below, and Formula 4-2 above is represented by 4-b or 4-c below: 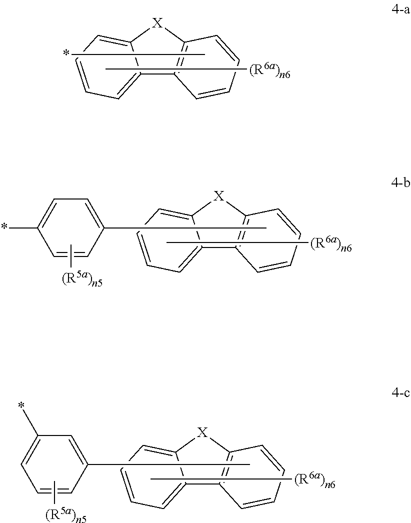
wherein, in 4-a to 4-c above,
R^{6a} is a hydrogen atom, a deuterium atom, or a substituted or unsubstituted aryl group having 6 to 15 ring-forming carbon atoms, or adjacent R^{6a}'s are bonded to each other to form an aromatic ring, and
X, n5, n6 and R^{5a} are the same as defined with respect to Formula 4-1 and Formula 4-2 above.

10. The light emitting element of claim 1, wherein Formula 5 above is represented by any one selected from among Formula 5-1 to Formula 5-4 below: 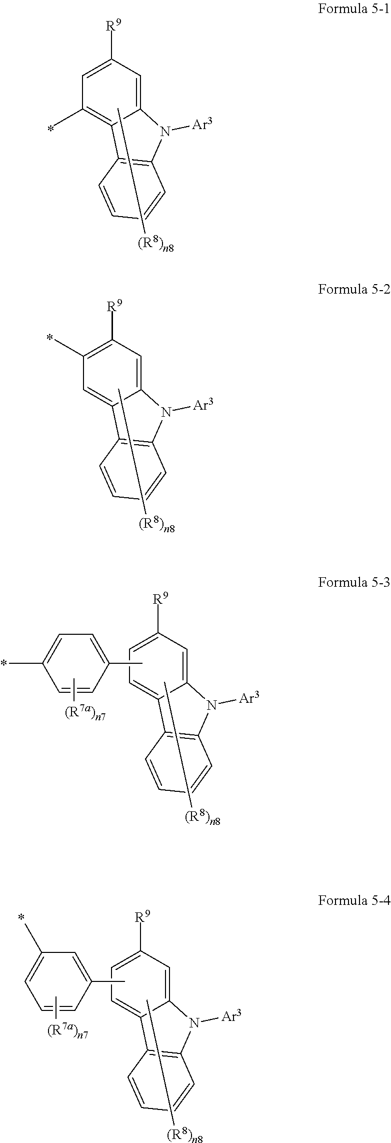
wherein, in Formula 5-1 to Formula 5-4 above,
R^{7a} is a hydrogen atom or a deuterium atom, and
n7, n8, R⁸, R⁹, and Ar₃ are the same as defined with respect to Formula 5 above.

11. The light emitting element of claim 10, wherein Formula 5-1 above is represented by 5-a below,
Formula 5-2 above is represented by 5-b below,
Formula 5-3 above is represented by 5-c or 5-d below, and
Formula 5-4 above is represented by 5-e or 5-f below: 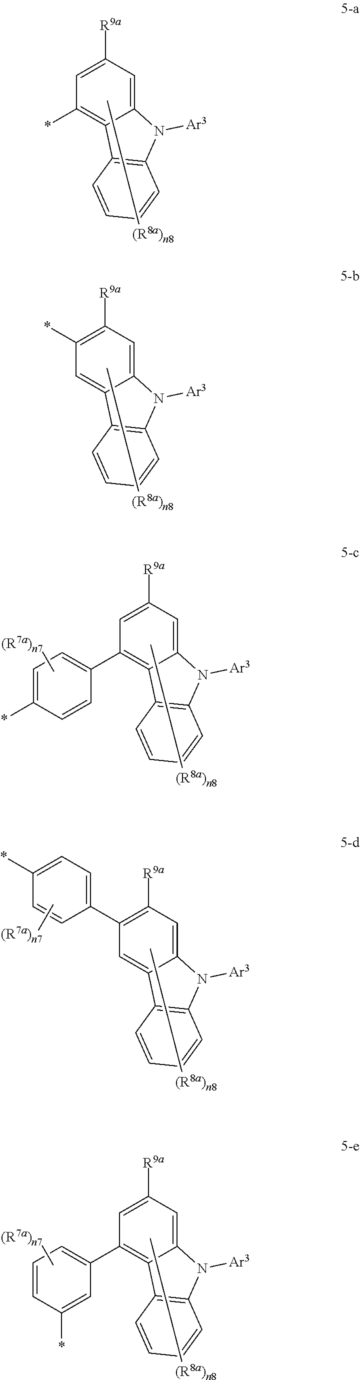 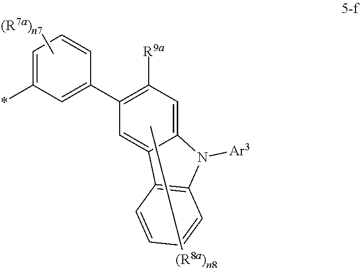
wherein, in 5-a to 5-f above,
R^{8a} is a hydrogen atom, a deuterium atom, a substituted or unsubstituted aryl group having 6 to 15 ring-forming carbon atoms, or adjacent R^{8a}'s are bonded to each other to form an aromatic ring,
R^{9a} is a hydrogen atom or a deuterium atom, and
n7, n8, R^{7a}, and Ar³ are the same as defined with respect to Formula 5-1 to Formula 5-4 above.

12. The light emitting element of claim 1, wherein R above is a hydrogen atom, a deuterium atom, a halogen atom, or a substituted or unsubstituted t-butyl group.

13. The light emitting element of claim 2, wherein the emission layer comprises a compound represented by Formula E-1 below: 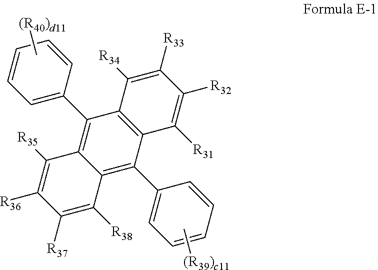
wherein, in Formula E-1, R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and
c and d are each independently an integer of 0 to 5.

14. The light emitting element of claim 1, wherein the amine compound is represented by any one selected from among compounds of Compound Group 1 below:

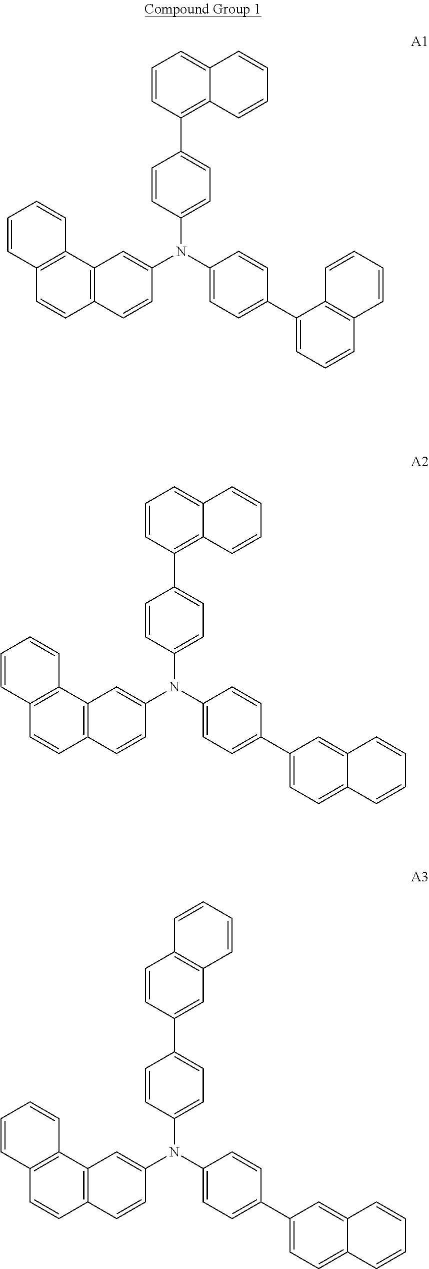 [SMILES: c1ccc2c(-c3ccc(N(c4ccc(-c5cccc6ccccc56)cc4)c4ccc5ccc6ccccc6c5c4)cc3)cccc2c1 ; c1ccc2cc(-c3ccc(N(c4ccc(-c5cccc6ccccc56)cc4)c4ccc5ccc6ccccc6c5c4)cc3)ccc2c1 ; c1ccc2cc(-c3ccc(N(c4ccc(-c5ccc6ccccc6c5)cc4)c4ccc5ccc6ccccc6c5c4)cc3)ccc2c1]

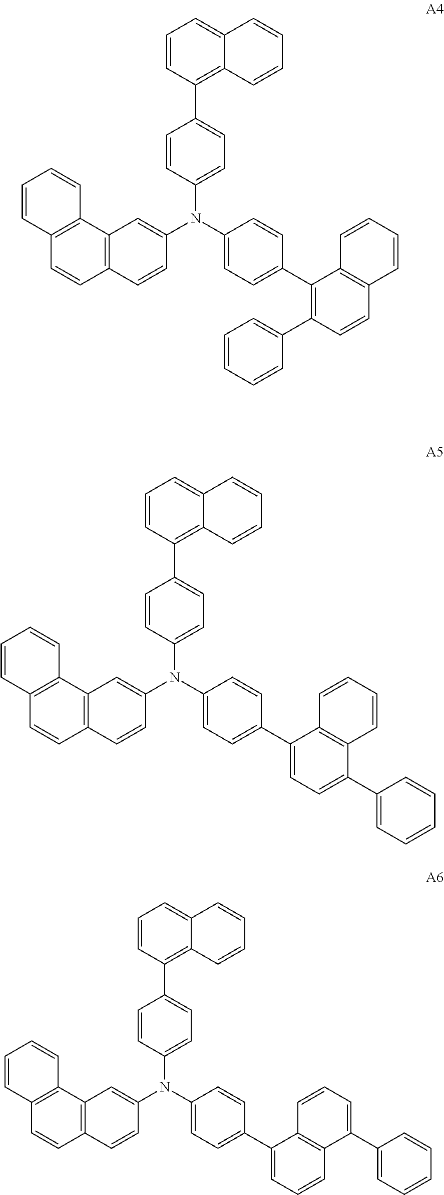 [SMILES: c1ccc(-c2ccc3ccccc3c2-c2ccc(N(c3ccc(-c4cccc5ccccc45)cc3)c3ccc4ccc5ccccc5c4c3)cc2)cc1 ; c1ccc(-c2ccc(-c3ccc(N(c4ccc(-c5cccc6ccccc56)cc4)c4ccc5ccc6ccccc6c5c4)cc3)c3ccccc23)cc1 ; c1ccc(-c2cccc3c(-c4ccc(N(c5ccc(-c6cccc7ccccc67)cc5)c5ccc6ccc7ccccc7c6c5)cc4)cccc23)cc1]

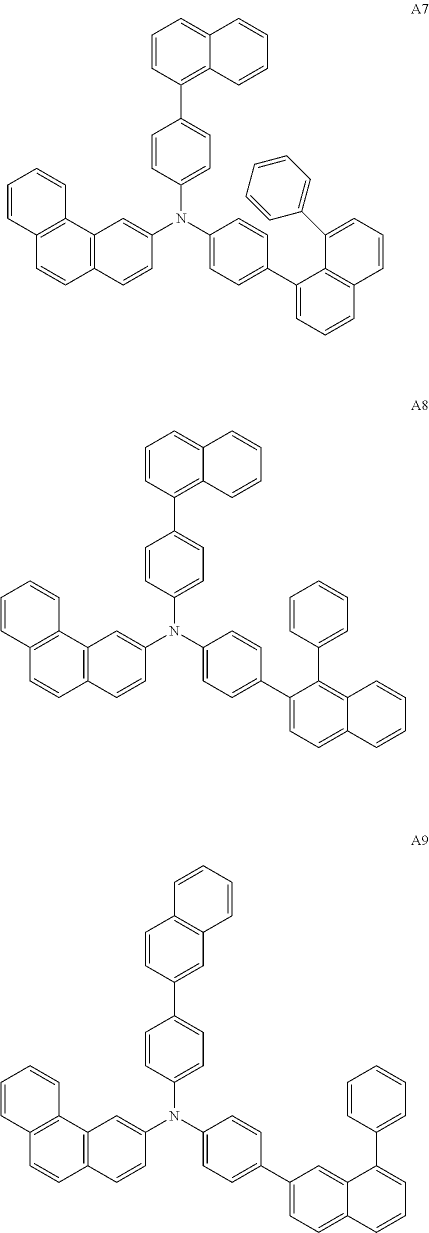 [SMILES: c1ccc(-c2cccc3cccc(-c4ccc(N(c5ccc(-c6cccc7ccccc67)cc5)c5ccc6ccc7ccccc7c6c5)cc4)c23)cc1 ; c1ccc(-c2c(-c3ccc(N(c4ccc(-c5cccc6ccccc56)cc4)c4ccc5ccc6ccccc6c5c4)cc3)ccc3ccccc23)cc1 ; c1ccc(-c2cccc3ccc(-c4ccc(N(c5ccc(-c6ccc7ccccc7c6)cc5)c5ccc6ccc7ccccc7c6c5)cc4)cc23)cc1]

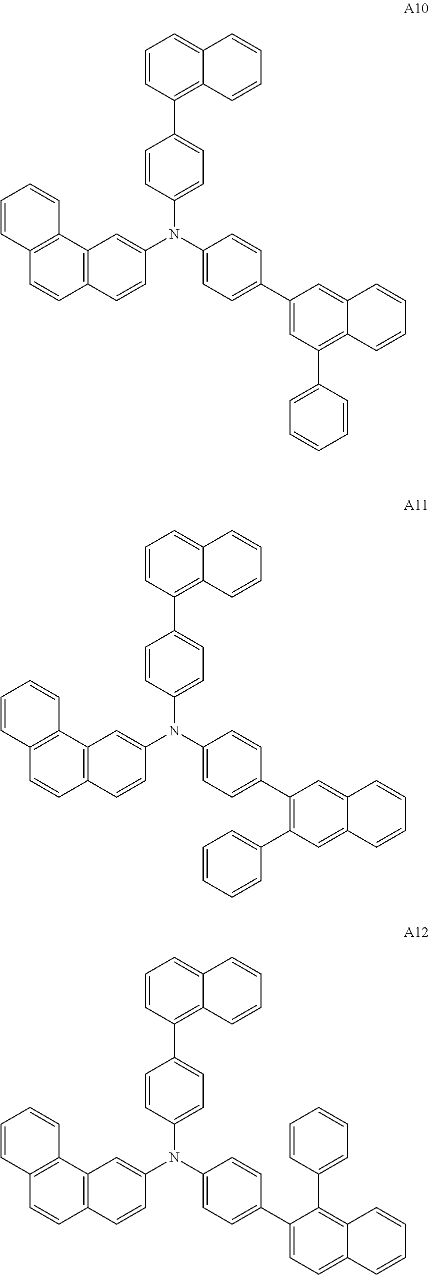 [SMILES: c1ccc(-c2cc(-c3ccc(N(c4ccc(-c5cccc6ccccc56)cc4)c4ccc5ccc6ccccc6c5c4)cc3)cc3ccccc23)cc1 ; c1ccc(-c2cc3ccccc3cc2-c2ccc(N(c3ccc(-c4cccc5ccccc45)cc3)c3ccc4ccc5ccccc5c4c3)cc2)cc1 ; c1ccc(-c2c(-c3ccc(N(c4ccc(-c5cccc6ccccc56)cc4)c4ccc5ccc6ccccc6c5c4)cc3)ccc3ccccc23)cc1]

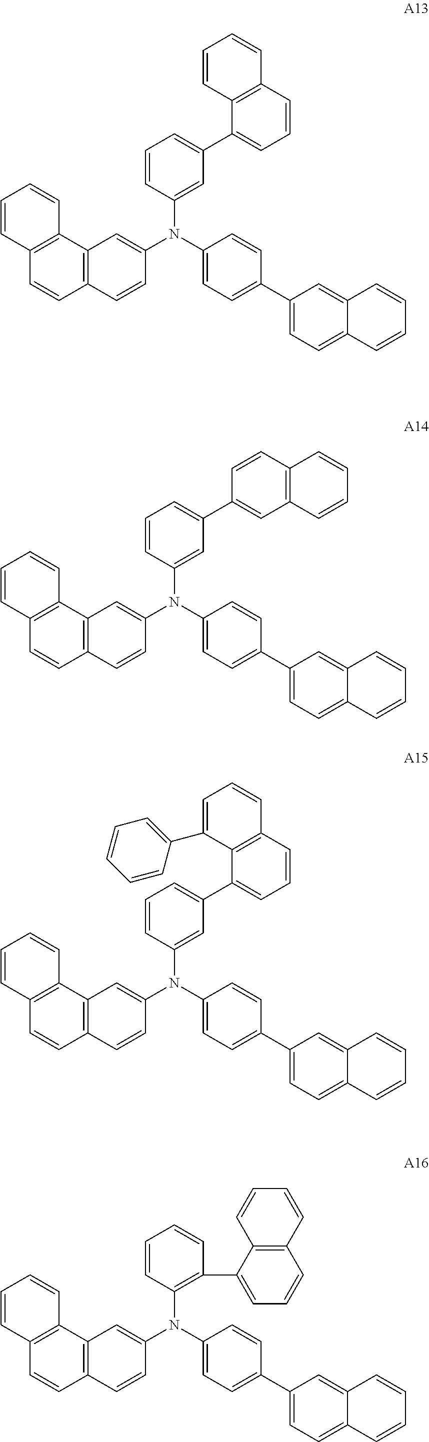 [SMILES: c1cc(-c2cccc3ccccc23)cc(N(c2ccc(-c3ccc4ccccc4c3)cc2)c2ccc3ccc4ccccc4c3c2)c1 ; c1cc(-c2ccc3ccccc3c2)cc(N(c2ccc(-c3ccc4ccccc4c3)cc2)c2ccc3ccc4ccccc4c3c2)c1 ; c1ccc(-c2cccc3cccc(-c4cccc(N(c5ccc(-c6ccc7ccccc7c6)cc5)c5ccc6ccc7ccccc7c6c5)c4)c23)cc1 ; c1ccc(N(c2ccc(-c3ccc4ccccc4c3)cc2)c2ccc3ccc4ccccc4c3c2)c(-c2cccc3ccccc23)c1]

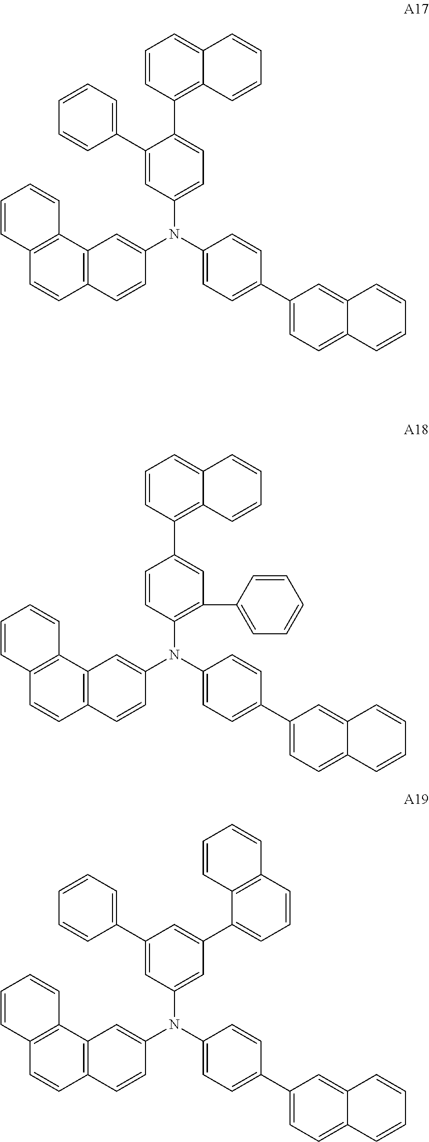 [SMILES: c1ccc(-c2cc(N(c3ccc(-c4ccc5ccccc5c4)cc3)c3ccc4ccc5ccccc5c4c3)ccc2-c2cccc3ccccc23)cc1 ; c1ccc(-c2cc(-c3cccc4ccccc34)ccc2N(c2ccc(-c3ccc4ccccc4c3)cc2)c2ccc3ccc4ccccc4c3c2)cc1 ; c1ccc(-c2cc(-c3cccc4ccccc34)cc(N(c3ccc(-c4ccc5ccccc5c4)cc3)c3ccc4ccc5ccccc5c4c3)c2)cc1]

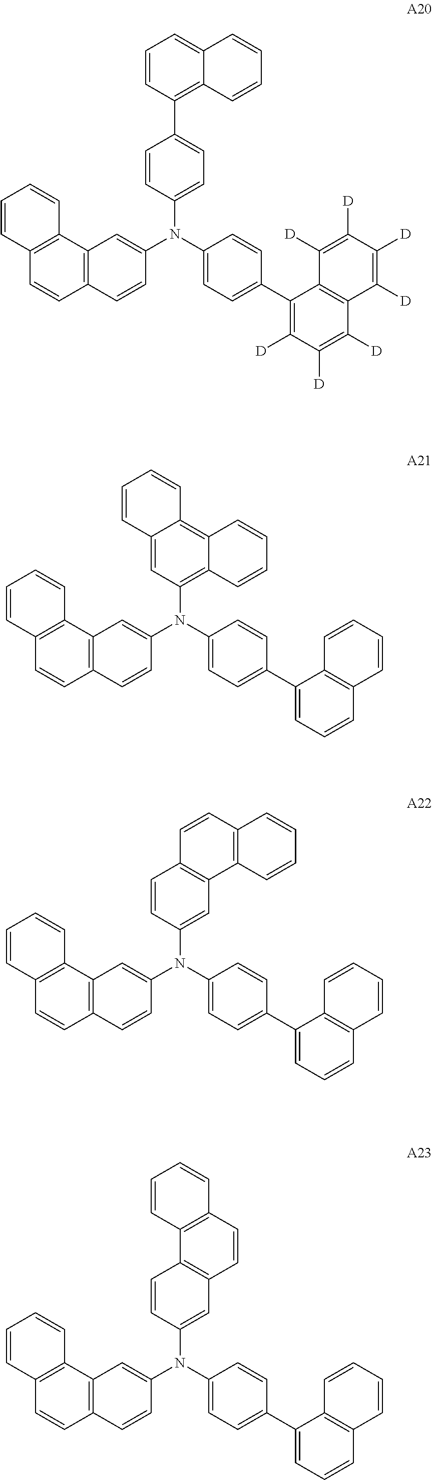 [SMILES: [2H]c1c([2H])c([2H])c2c(-c3ccc(N(c4ccc(-c5cccc6ccccc56)cc4)c4ccc5ccc6ccccc6c5c4)cc3)c([2H])c([2H])c([2H])c2c1[2H] ; c1ccc2c(-c3ccc(N(c4ccc5ccc6ccccc6c5c4)c4cc5ccccc5c5ccccc45)cc3)cccc2c1 ; c1ccc2c(-c3ccc(N(c4ccc5ccc6ccccc6c5c4)c4ccc5ccc6ccccc6c5c4)cc3)cccc2c1 ; c1ccc2c(-c3ccc(N(c4ccc5c(ccc6ccccc65)c4)c4ccc5ccc6ccccc6c5c4)cc3)cccc2c1]

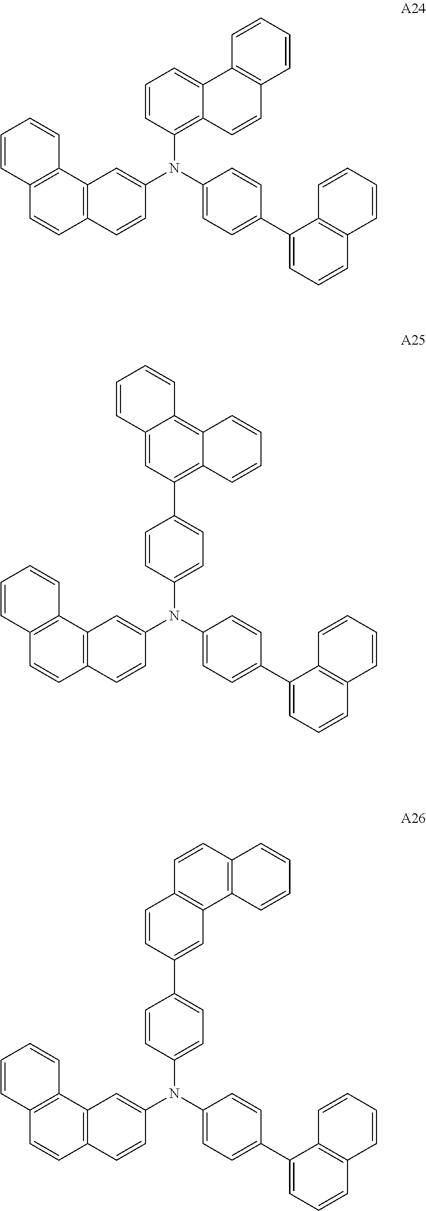 [SMILES: c1ccc2c(-c3ccc(N(c4ccc5ccc6ccccc6c5c4)c4cccc5c4ccc4ccccc45)cc3)cccc2c1 ; c1ccc2c(-c3ccc(N(c4ccc(-c5cc6ccccc6c6ccccc56)cc4)c4ccc5ccc6ccccc6c5c4)cc3)cccc2c1 ; c1ccc2c(-c3ccc(N(c4ccc(-c5ccc6ccc7ccccc7c6c5)cc4)c4ccc5ccc6ccccc6c5c4)cc3)cccc2c1]

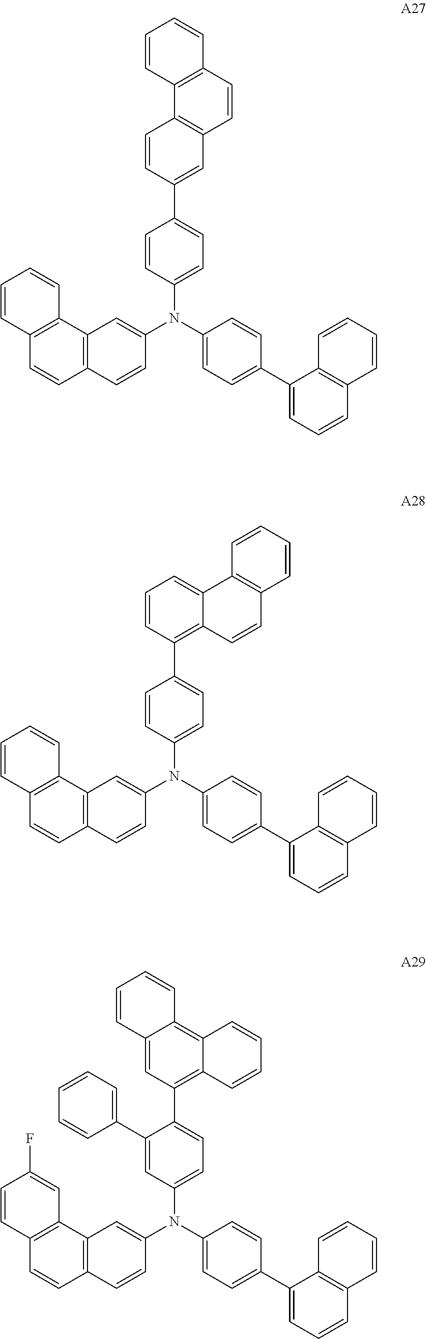 [SMILES: c1ccc2c(-c3ccc(N(c4ccc(-c5ccc6c(ccc7ccccc76)c5)cc4)c4ccc5ccc6ccccc6c5c4)cc3)cccc2c1 ; c1ccc2c(-c3ccc(N(c4ccc(-c5cccc6c5ccc5ccccc56)cc4)c4ccc5ccc6ccccc6c5c4)cc3)cccc2c1 ; Fc1ccc2ccc3ccc(N(c4ccc(-c5cccc6ccccc56)cc4)c4ccc(-c5cc6ccccc6c6ccccc56)c(-c5ccccc5)c4)cc3c2c1]

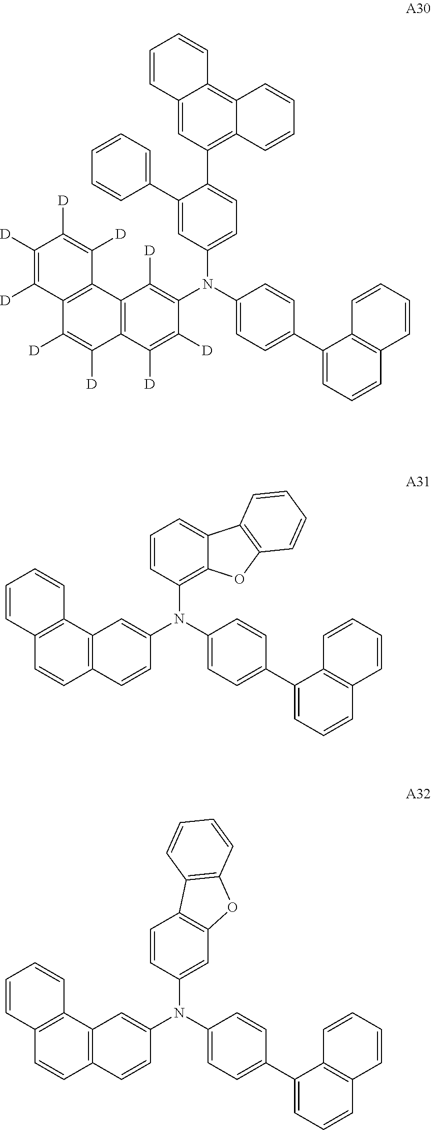 [SMILES: [2H]c1c([2H])c([2H])c2c(c1[2H])c([2H])c([2H])c1c([2H])c([2H])c(N(c3ccc(-c4cccc5ccccc45)cc3)c3ccc(-c4cc5ccccc5c5ccccc45)c(-c4ccccc4)c3)c([2H])c12 ; c1ccc2c(-c3ccc(N(c4ccc5ccc6ccccc6c5c4)c4cccc5c4oc4ccccc45)cc3)cccc2c1 ; c1ccc2c(-c3ccc(N(c4ccc5c(c4)oc4ccccc45)c4ccc5ccc6ccccc6c5c4)cc3)cccc2c1]

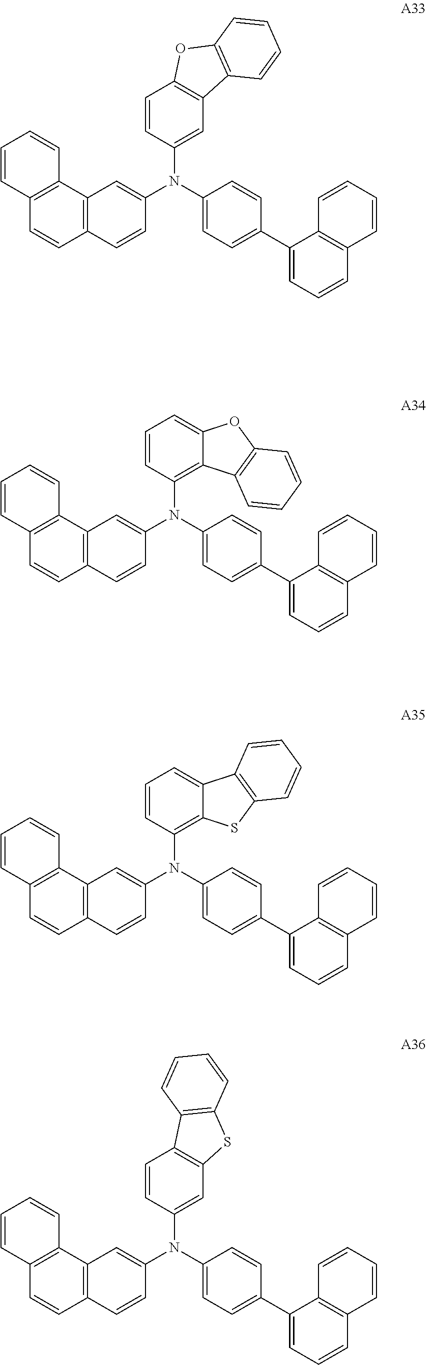 [SMILES: c1ccc2c(-c3ccc(N(c4ccc5ccc6ccccc6c5c4)c4ccc5oc6ccccc6c5c4)cc3)cccc2c1 ; c1ccc2c(-c3ccc(N(c4ccc5ccc6ccccc6c5c4)c4cccc5oc6ccccc6c45)cc3)cccc2c1 ; c1ccc2c(-c3ccc(N(c4ccc5ccc6ccccc6c5c4)c4cccc5c4sc4ccccc45)cc3)cccc2c1 ; c1ccc2c(-c3ccc(N(c4ccc5c(c4)sc4ccccc45)c4ccc5ccc6ccccc6c5c4)cc3)cccc2c1]

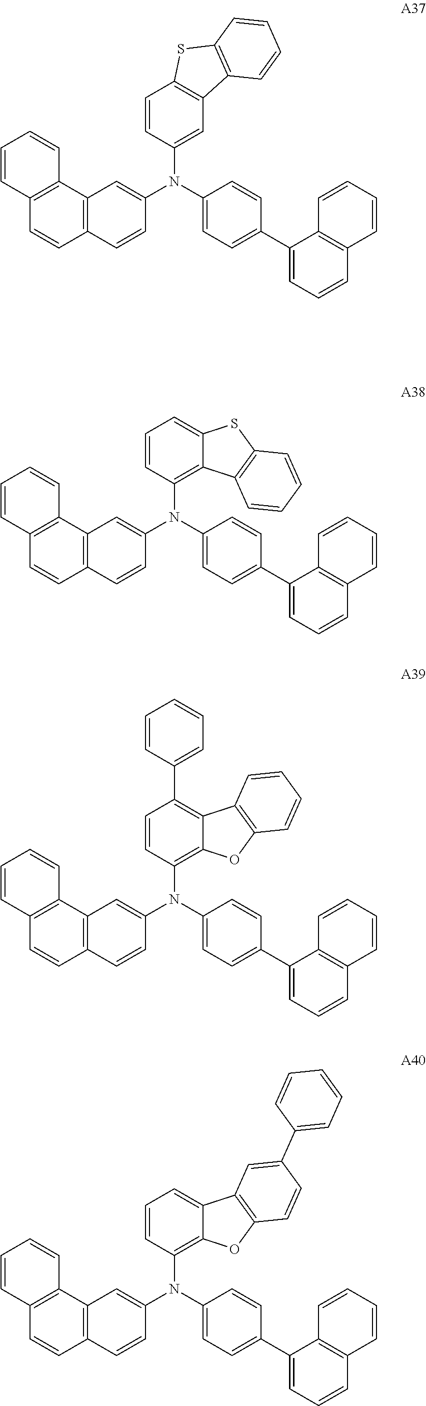 [SMILES: c1ccc2c(-c3ccc(N(c4ccc5ccc6ccccc6c5c4)c4ccc5sc6ccccc6c5c4)cc3)cccc2c1 ; c1ccc2c(-c3ccc(N(c4ccc5ccc6ccccc6c5c4)c4cccc5sc6ccccc6c45)cc3)cccc2c1 ; c1ccc(-c2ccc(N(c3ccc(-c4cccc5ccccc45)cc3)c3ccc4ccc5ccccc5c4c3)c3oc4ccccc4c23)cc1 ; c1ccc(-c2ccc3oc4c(N(c5ccc(-c6cccc7ccccc67)cc5)c5ccc6ccc7ccccc7c6c5)cccc4c3c2)cc1]

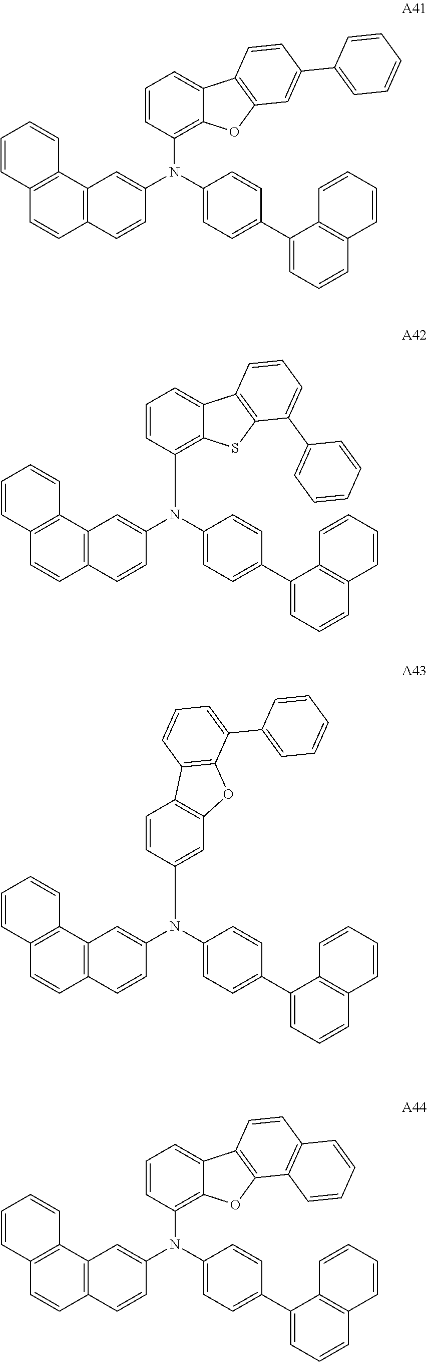 [SMILES: c1ccc(-c2ccc3c(c2)oc2c(N(c4ccc(-c5cccc6ccccc56)cc4)c4ccc5ccc6ccccc6c5c4)cccc23)cc1 ; c1ccc(-c2cccc3c2sc2c(N(c4ccc(-c5cccc6ccccc56)cc4)c4ccc5ccc6ccccc6c5c4)cccc23)cc1 ; c1ccc(-c2cccc3c2oc2cc(N(c4ccc(-c5cccc6ccccc56)cc4)c4ccc5ccc6ccccc6c5c4)ccc23)cc1 ; c1ccc2c(-c3ccc(N(c4ccc5ccc6ccccc6c5c4)c4cccc5c4oc4c6ccccc6ccc54)cc3)cccc2c1]

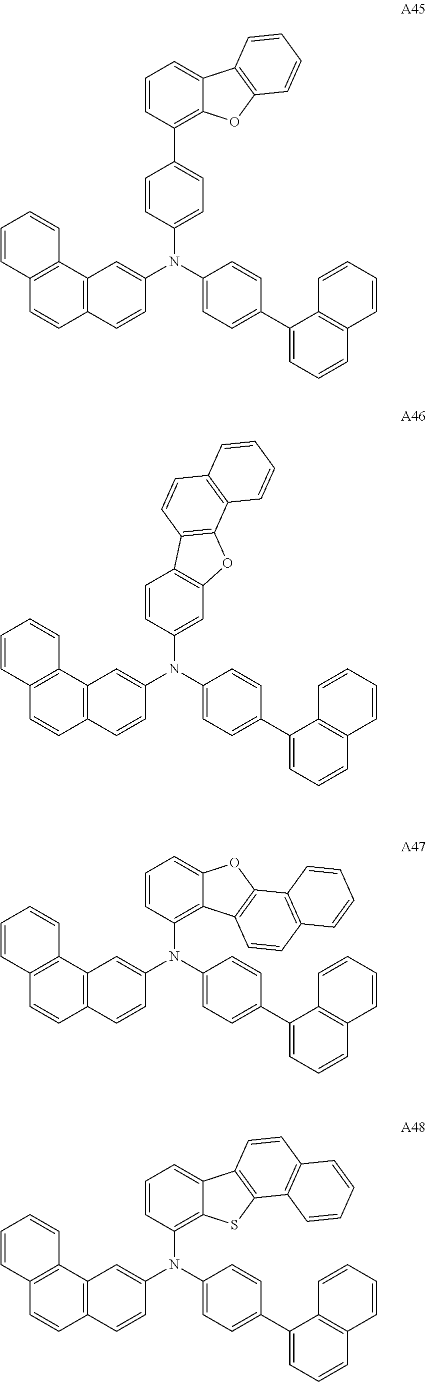 [SMILES: c1ccc2c(-c3ccc(N(c4ccc(-c5cccc6c5oc5ccccc56)cc4)c4ccc5ccc6ccccc6c5c4)cc3)cccc2c1 ; c1ccc2c(-c3ccc(N(c4ccc5c(c4)oc4c6ccccc6ccc54)c4ccc5ccc6ccccc6c5c4)cc3)cccc2c1 ; c1ccc2c(-c3ccc(N(c4ccc5ccc6ccccc6c5c4)c4cccc5oc6c7ccccc7ccc6c45)cc3)cccc2c1 ; c1ccc2c(-c3ccc(N(c4ccc5ccc6ccccc6c5c4)c4cccc5c4sc4c6ccccc6ccc54)cc3)cccc2c1]

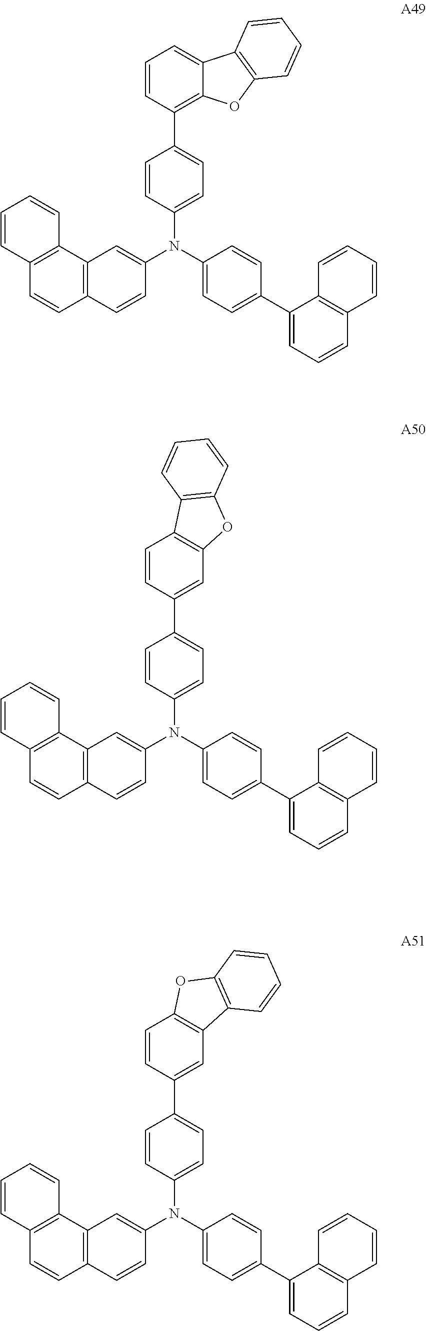 [SMILES: c1ccc2c(-c3ccc(N(c4ccc(-c5cccc6c5oc5ccccc56)cc4)c4ccc5ccc6ccccc6c5c4)cc3)cccc2c1 ; c1ccc2c(-c3ccc(N(c4ccc(-c5ccc6c(c5)oc5ccccc56)cc4)c4ccc5ccc6ccccc6c5c4)cc3)cccc2c1 ; c1ccc2c(-c3ccc(N(c4ccc(-c5ccc6oc7ccccc7c6c5)cc4)c4ccc5ccc6ccccc6c5c4)cc3)cccc2c1]

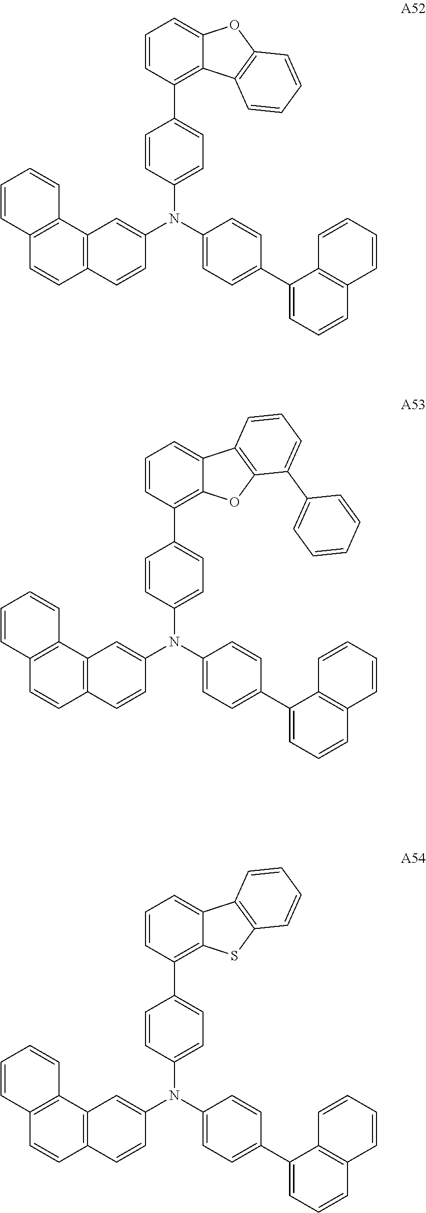 [SMILES: c1ccc2c(-c3ccc(N(c4ccc(-c5cccc6oc7ccccc7c56)cc4)c4ccc5ccc6ccccc6c5c4)cc3)cccc2c1 ; c1ccc(-c2cccc3c2oc2c(-c4ccc(N(c5ccc(-c6cccc7ccccc67)cc5)c5ccc6ccc7ccccc7c6c5)cc4)cccc23)cc1 ; c1ccc2c(-c3ccc(N(c4ccc(-c5cccc6c5sc5ccccc56)cc4)c4ccc5ccc6ccccc6c5c4)cc3)cccc2c1]

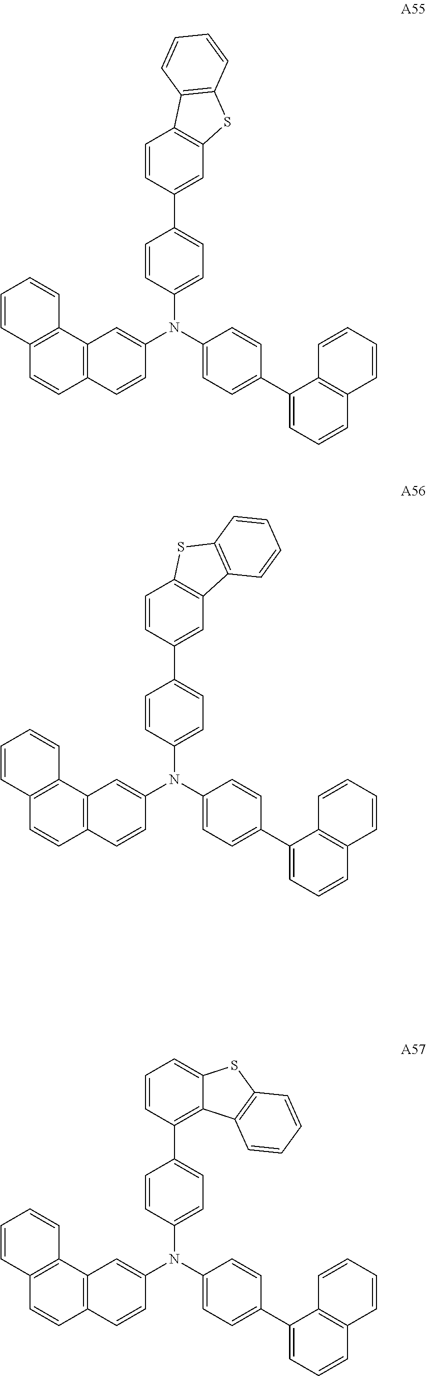 [SMILES: c1ccc2c(-c3ccc(N(c4ccc(-c5ccc6c(c5)sc5ccccc56)cc4)c4ccc5ccc6ccccc6c5c4)cc3)cccc2c1 ; c1ccc2c(-c3ccc(N(c4ccc(-c5ccc6sc7ccccc7c6c5)cc4)c4ccc5ccc6ccccc6c5c4)cc3)cccc2c1 ; c1ccc2c(-c3ccc(N(c4ccc(-c5cccc6sc7ccccc7c56)cc4)c4ccc5ccc6ccccc6c5c4)cc3)cccc2c1]

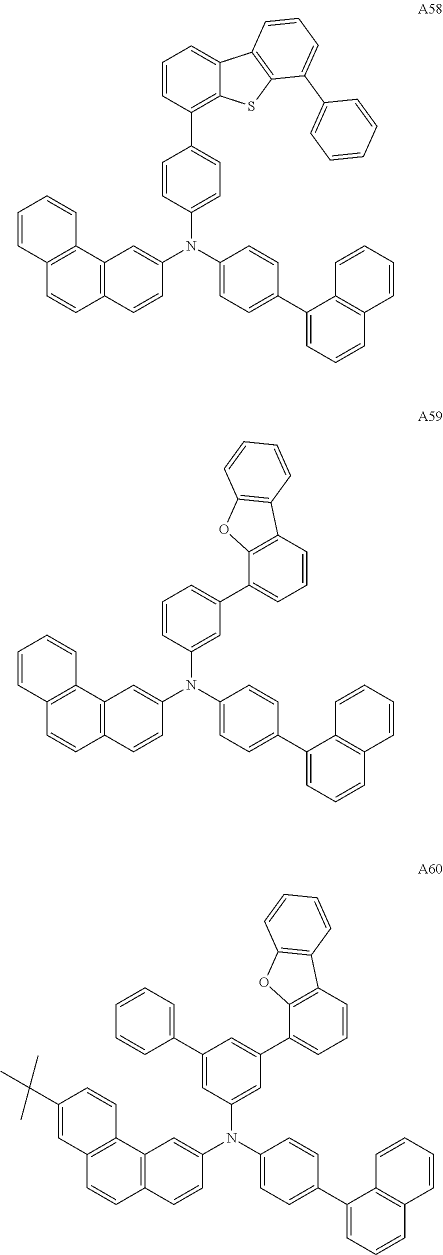 [SMILES: c1ccc(-c2cccc3c2sc2c(-c4ccc(N(c5ccc(-c6cccc7ccccc67)cc5)c5ccc6ccc7ccccc7c6c5)cc4)cccc23)cc1 ; c1cc(-c2cccc3c2oc2ccccc23)cc(N(c2ccc(-c3cccc4ccccc34)cc2)c2ccc3ccc4ccccc4c3c2)c1 ; CC(C)(C)c1ccc2c(ccc3ccc(N(c4ccc(-c5cccc6ccccc56)cc4)c4cc(-c5ccccc5)cc(-c5cccc6c5oc5ccccc56)c4)cc32)c1]

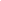

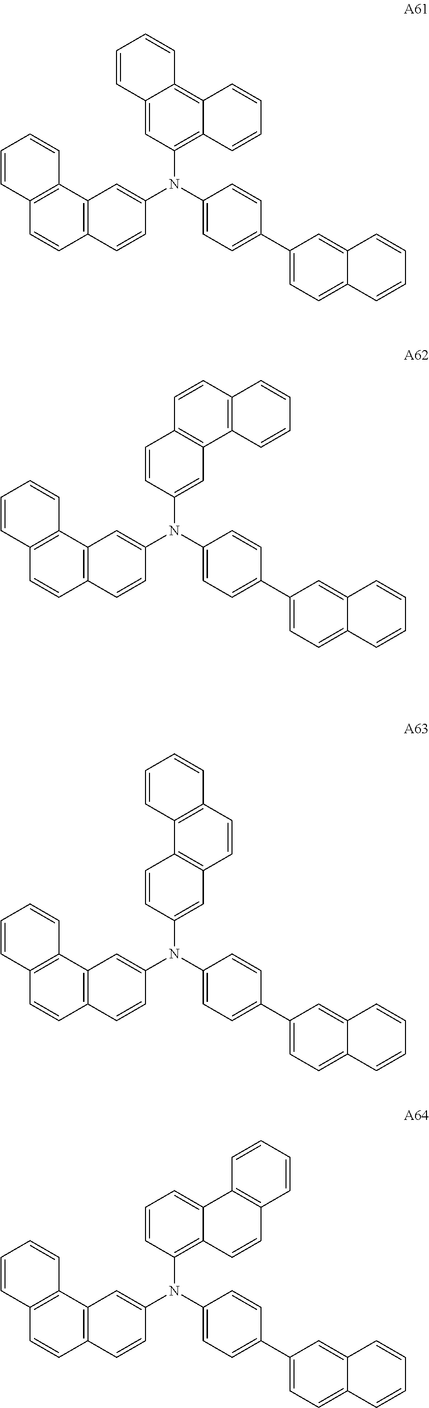 [SMILES: c1ccc2cc(-c3ccc(N(c4ccc5ccc6ccccc6c5c4)c4cc5ccccc5c5ccccc45)cc3)ccc2c1 ; c1ccc2cc(-c3ccc(N(c4ccc5ccc6ccccc6c5c4)c4ccc5ccc6ccccc6c5c4)cc3)ccc2c1 ; c1ccc2cc(-c3ccc(N(c4ccc5c(ccc6ccccc65)c4)c4ccc5ccc6ccccc6c5c4)cc3)ccc2c1 ; c1ccc2cc(-c3ccc(N(c4ccc5ccc6ccccc6c5c4)c4cccc5c4ccc4ccccc45)cc3)ccc2c1]

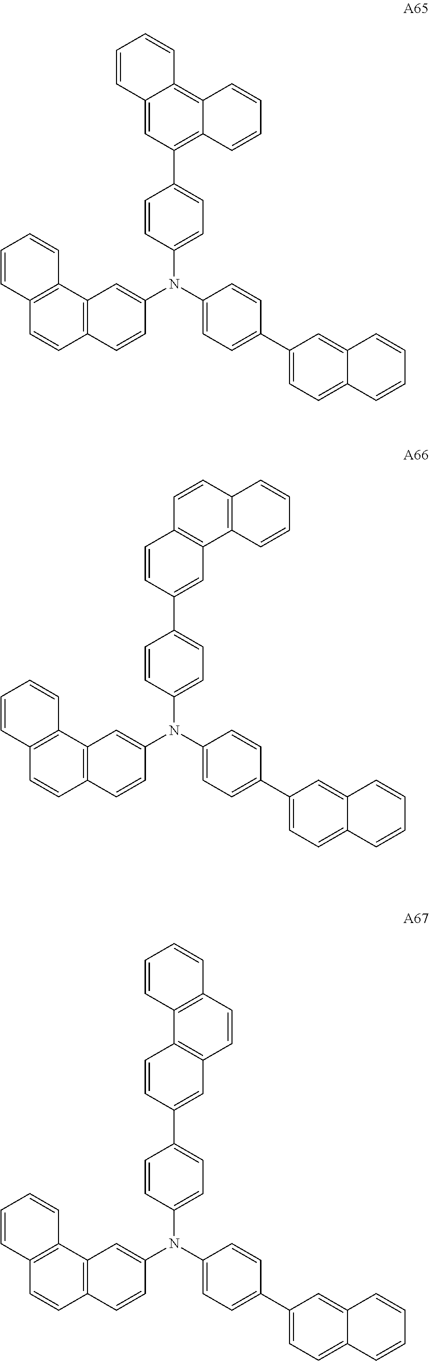 [SMILES: c1ccc2cc(-c3ccc(N(c4ccc(-c5cc6ccccc6c6ccccc56)cc4)c4ccc5ccc6ccccc6c5c4)cc3)ccc2c1 ; c1ccc2cc(-c3ccc(N(c4ccc(-c5ccc6ccc7ccccc7c6c5)cc4)c4ccc5ccc6ccccc6c5c4)cc3)ccc2c1 ; c1ccc2cc(-c3ccc(N(c4ccc(-c5ccc6c(ccc7ccccc76)c5)cc4)c4ccc5ccc6ccccc6c5c4)cc3)ccc2c1]

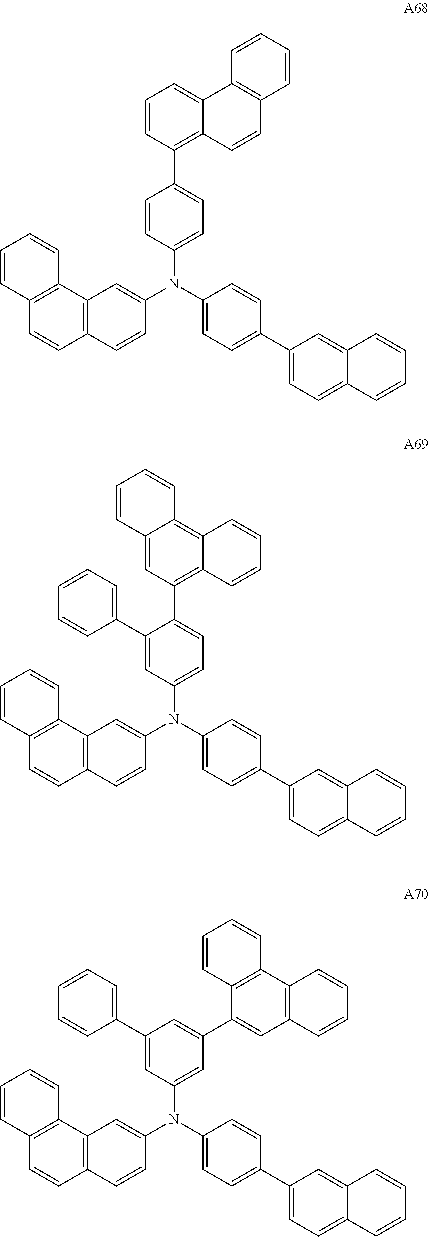 [SMILES: c1ccc2cc(-c3ccc(N(c4ccc(-c5cccc6c5ccc5ccccc56)cc4)c4ccc5ccc6ccccc6c5c4)cc3)ccc2c1 ; c1ccc(-c2cc(N(c3ccc(-c4ccc5ccccc5c4)cc3)c3ccc4ccc5ccccc5c4c3)ccc2-c2cc3ccccc3c3ccccc23)cc1 ; c1ccc(-c2cc(-c3cc4ccccc4c4ccccc34)cc(N(c3ccc(-c4ccc5ccccc5c4)cc3)c3ccc4ccc5ccccc5c4c3)c2)cc1]

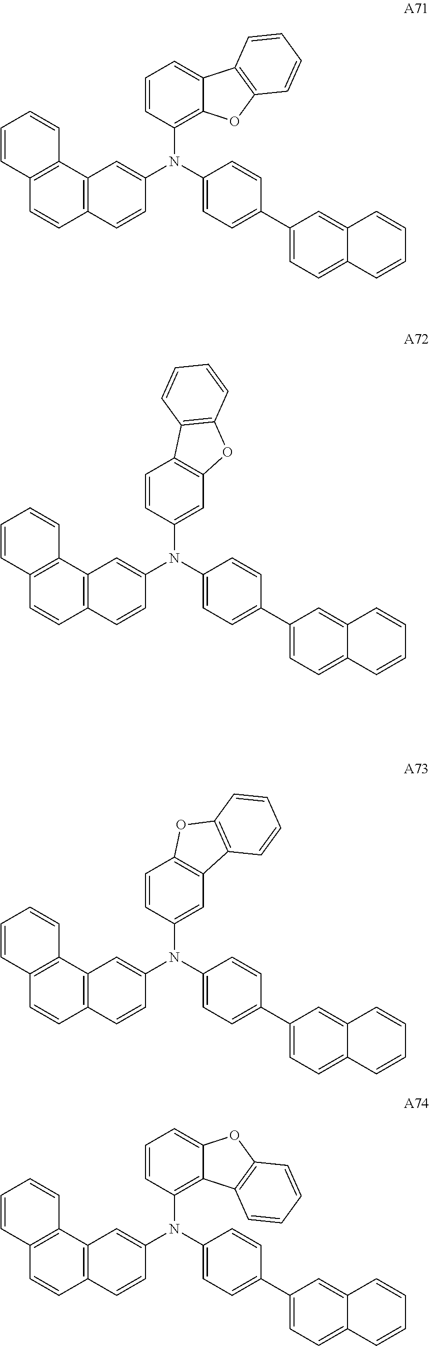 [SMILES: c1ccc2cc(-c3ccc(N(c4ccc5ccc6ccccc6c5c4)c4cccc5c4oc4ccccc45)cc3)ccc2c1 ; c1ccc2cc(-c3ccc(N(c4ccc5c(c4)oc4ccccc45)c4ccc5ccc6ccccc6c5c4)cc3)ccc2c1 ; c1ccc2cc(-c3ccc(N(c4ccc5ccc6ccccc6c5c4)c4ccc5oc6ccccc6c5c4)cc3)ccc2c1 ; c1ccc2cc(-c3ccc(N(c4ccc5ccc6ccccc6c5c4)c4cccc5oc6ccccc6c45)cc3)ccc2c1]

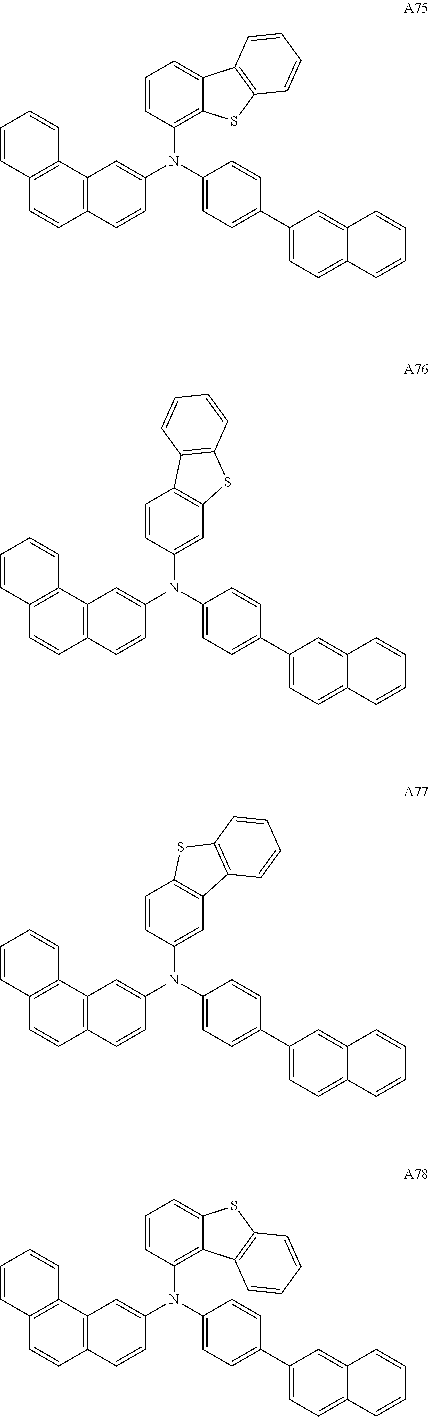 [SMILES: c1ccc2cc(-c3ccc(N(c4ccc5ccc6ccccc6c5c4)c4cccc5c4sc4ccccc45)cc3)ccc2c1 ; c1ccc2cc(-c3ccc(N(c4ccc5c(c4)sc4ccccc45)c4ccc5ccc6ccccc6c5c4)cc3)ccc2c1 ; c1ccc2cc(-c3ccc(N(c4ccc5ccc6ccccc6c5c4)c4ccc5sc6ccccc6c5c4)cc3)ccc2c1 ; c1ccc2cc(-c3ccc(N(c4ccc5ccc6ccccc6c5c4)c4cccc5sc6ccccc6c45)cc3)ccc2c1]

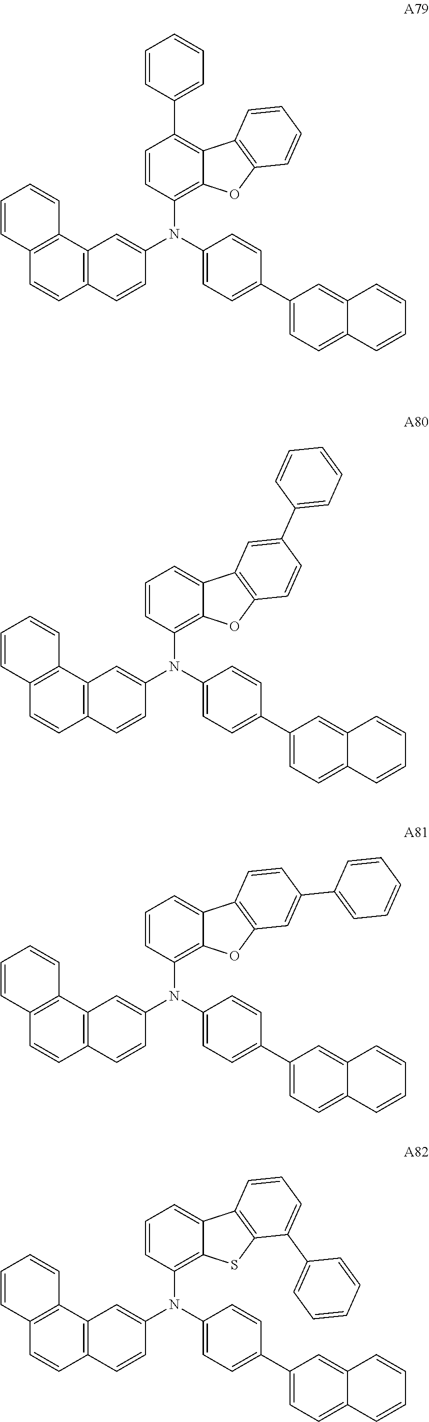 [SMILES: c1ccc(-c2ccc(N(c3ccc(-c4ccc5ccccc5c4)cc3)c3ccc4ccc5ccccc5c4c3)c3oc4ccccc4c23)cc1 ; c1ccc(-c2ccc3oc4c(N(c5ccc(-c6ccc7ccccc7c6)cc5)c5ccc6ccc7ccccc7c6c5)cccc4c3c2)cc1 ; c1ccc(-c2ccc3c(c2)oc2c(N(c4ccc(-c5ccc6ccccc6c5)cc4)c4ccc5ccc6ccccc6c5c4)cccc23)cc1 ; c1ccc(-c2cccc3c2sc2c(N(c4ccc(-c5ccc6ccccc6c5)cc4)c4ccc5ccc6ccccc6c5c4)cccc23)cc1]

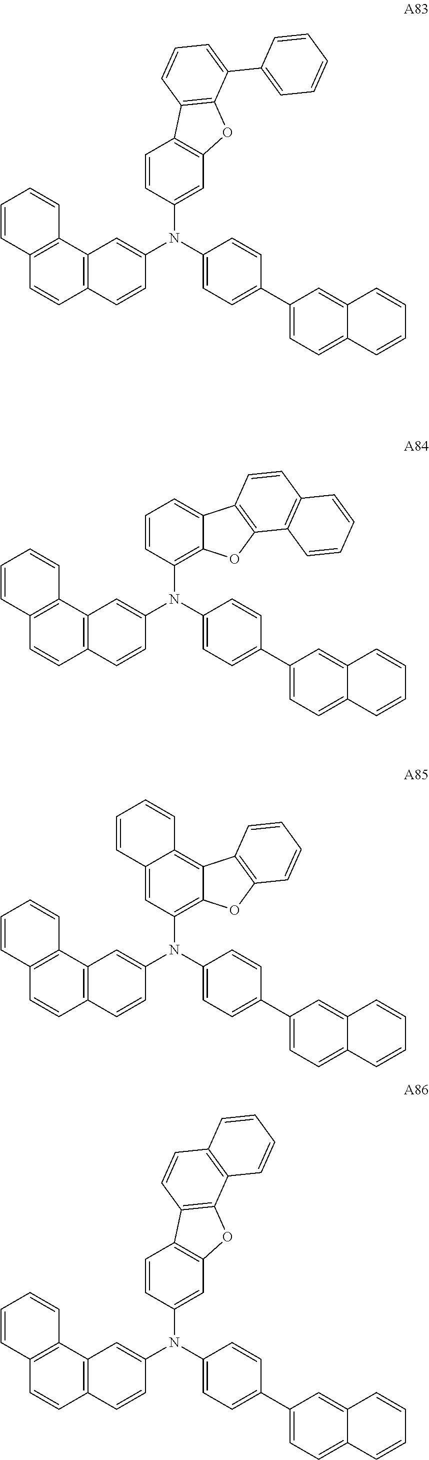 [SMILES: c1ccc(-c2cccc3c2oc2cc(N(c4ccc(-c5ccc6ccccc6c5)cc4)c4ccc5ccc6ccccc6c5c4)ccc23)cc1 ; c1ccc2cc(-c3ccc(N(c4ccc5ccc6ccccc6c5c4)c4cccc5c4oc4c6ccccc6ccc54)cc3)ccc2c1 ; c1ccc2cc(-c3ccc(N(c4ccc5ccc6ccccc6c5c4)c4cc5ccccc5c5c4oc4ccccc45)cc3)ccc2c1 ; c1ccc2cc(-c3ccc(N(c4ccc5c(c4)oc4c6ccccc6ccc54)c4ccc5ccc6ccccc6c5c4)cc3)ccc2c1]

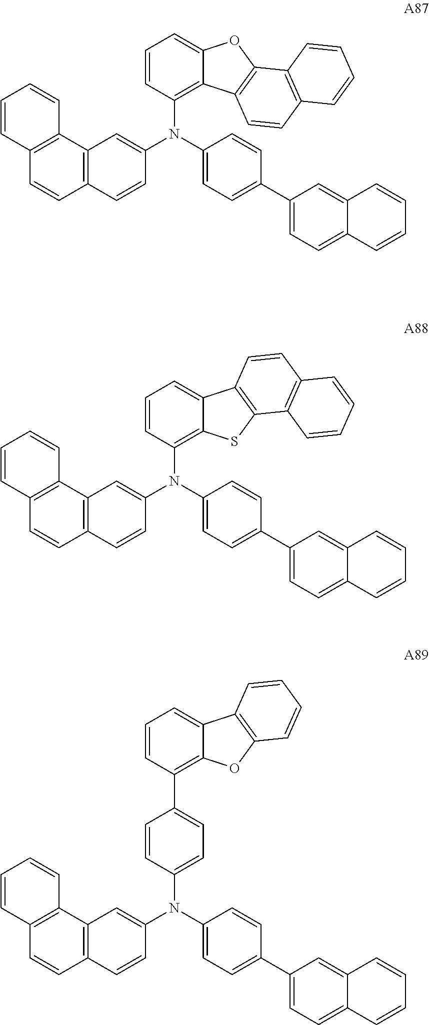 [SMILES: c1ccc2cc(-c3ccc(N(c4ccc5ccc6ccccc6c5c4)c4cccc5oc6c7ccccc7ccc6c45)cc3)ccc2c1 ; c1ccc2cc(-c3ccc(N(c4ccc5ccc6ccccc6c5c4)c4cccc5c4sc4c6ccccc6ccc54)cc3)ccc2c1 ; c1ccc2cc(-c3ccc(N(c4ccc(-c5cccc6c5oc5ccccc56)cc4)c4ccc5ccc6ccccc6c5c4)cc3)ccc2c1]

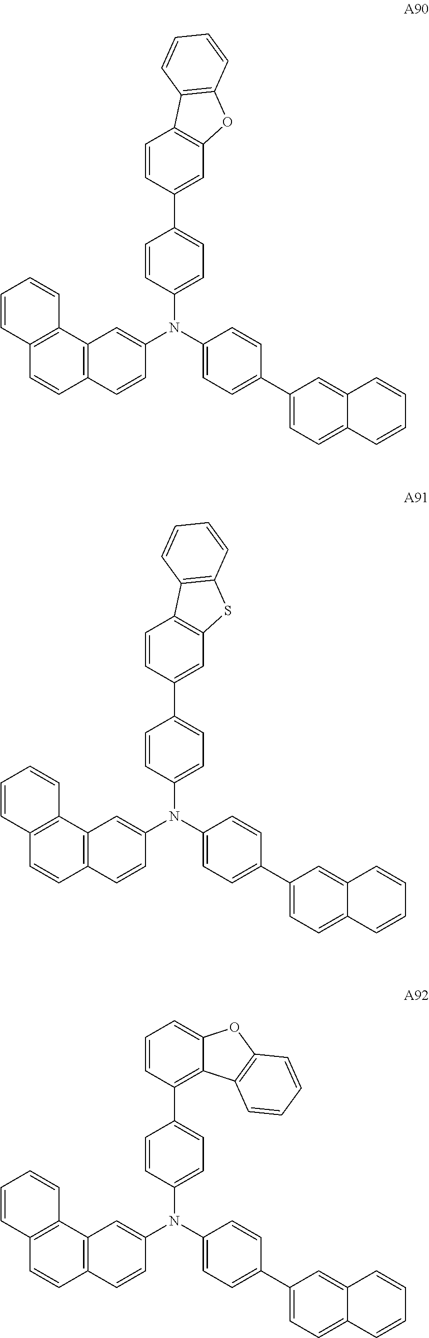 [SMILES: c1ccc2cc(-c3ccc(N(c4ccc(-c5ccc6c(c5)oc5ccccc56)cc4)c4ccc5ccc6ccccc6c5c4)cc3)ccc2c1 ; c1ccc2cc(-c3ccc(N(c4ccc(-c5ccc6c(c5)sc5ccccc56)cc4)c4ccc5ccc6ccccc6c5c4)cc3)ccc2c1 ; c1ccc2cc(-c3ccc(N(c4ccc(-c5cccc6oc7ccccc7c56)cc4)c4ccc5ccc6ccccc6c5c4)cc3)ccc2c1]

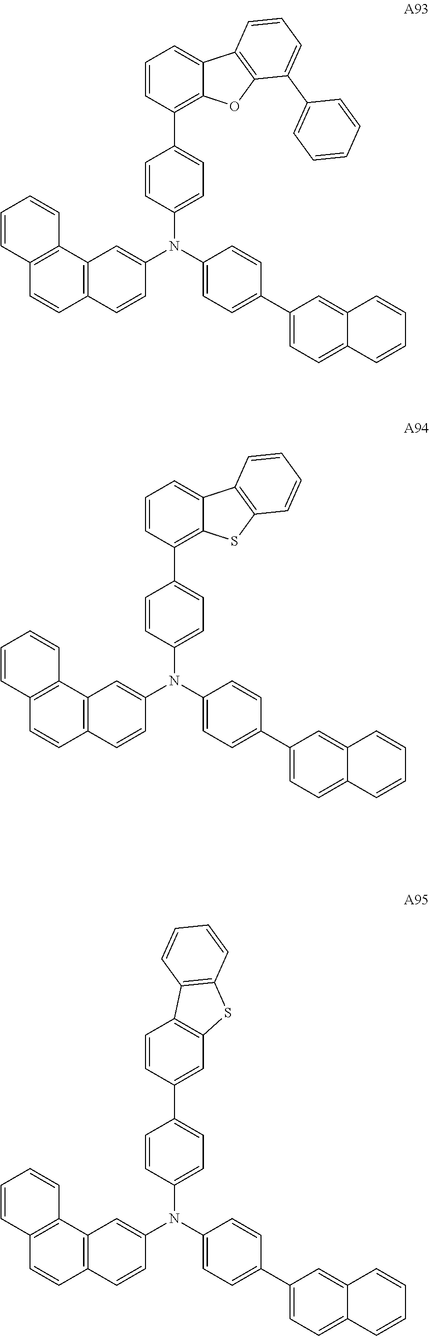 [SMILES: c1ccc(-c2cccc3c2oc2c(-c4ccc(N(c5ccc(-c6ccc7ccccc7c6)cc5)c5ccc6ccc7ccccc7c6c5)cc4)cccc23)cc1 ; c1ccc2cc(-c3ccc(N(c4ccc(-c5cccc6c5sc5ccccc56)cc4)c4ccc5ccc6ccccc6c5c4)cc3)ccc2c1 ; c1ccc2cc(-c3ccc(N(c4ccc(-c5ccc6c(c5)sc5ccccc56)cc4)c4ccc5ccc6ccccc6c5c4)cc3)ccc2c1]

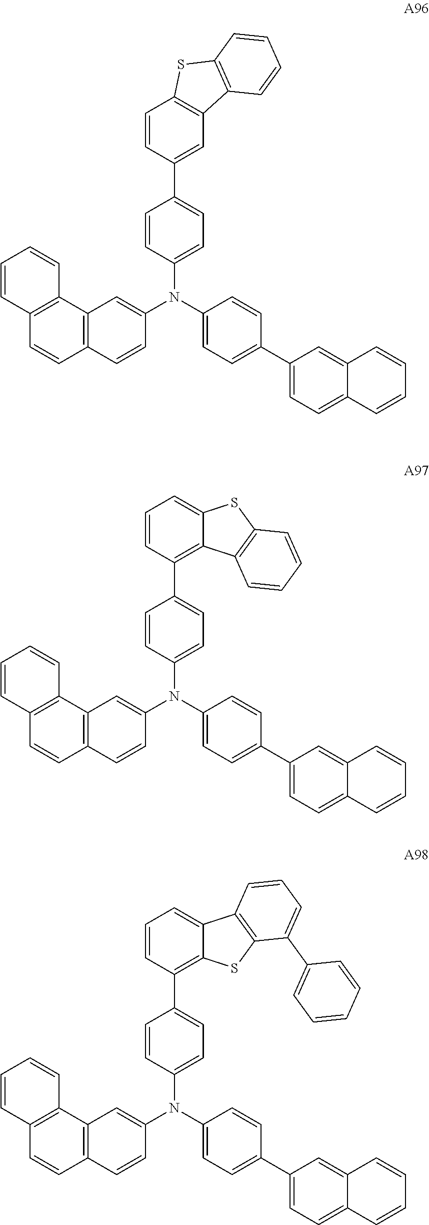 [SMILES: c1ccc2cc(-c3ccc(N(c4ccc(-c5ccc6sc7ccccc7c6c5)cc4)c4ccc5ccc6ccccc6c5c4)cc3)ccc2c1 ; c1ccc2cc(-c3ccc(N(c4ccc(-c5cccc6sc7ccccc7c56)cc4)c4ccc5ccc6ccccc6c5c4)cc3)ccc2c1 ; c1ccc(-c2cccc3c2sc2c(-c4ccc(N(c5ccc(-c6ccc7ccccc7c6)cc5)c5ccc6ccc7ccccc7c6c5)cc4)cccc23)cc1]

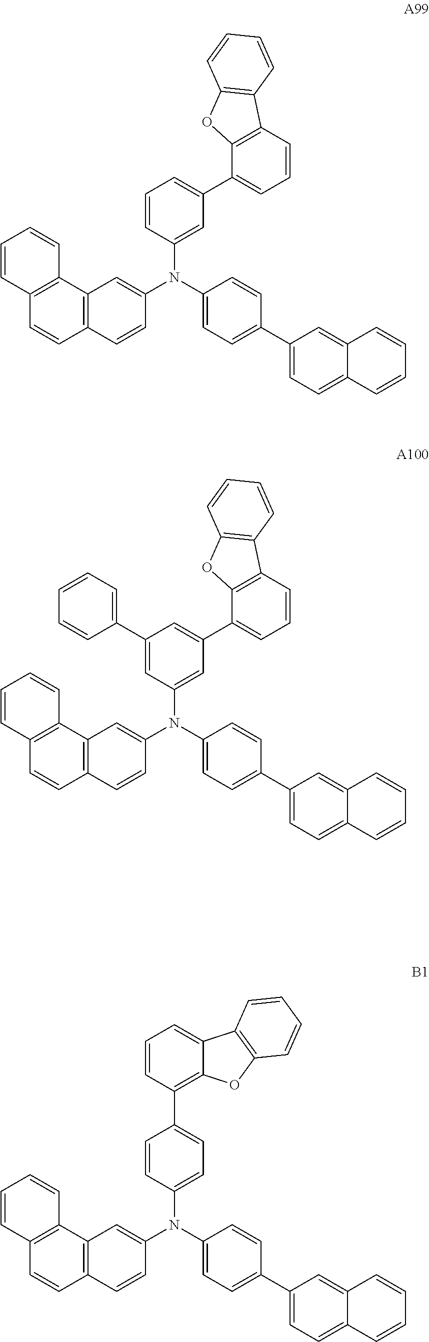 [SMILES: c1cc(-c2cccc3c2oc2ccccc23)cc(N(c2ccc(-c3ccc4ccccc4c3)cc2)c2ccc3ccc4ccccc4c3c2)c1 ; c1ccc(-c2cc(-c3cccc4c3oc3ccccc34)cc(N(c3ccc(-c4ccc5ccccc5c4)cc3)c3ccc4ccc5ccccc5c4c3)c2)cc1 ; c1ccc2cc(-c3ccc(N(c4ccc(-c5cccc6c5oc5ccccc56)cc4)c4ccc5ccc6ccccc6c5c4)cc3)ccc2c1]

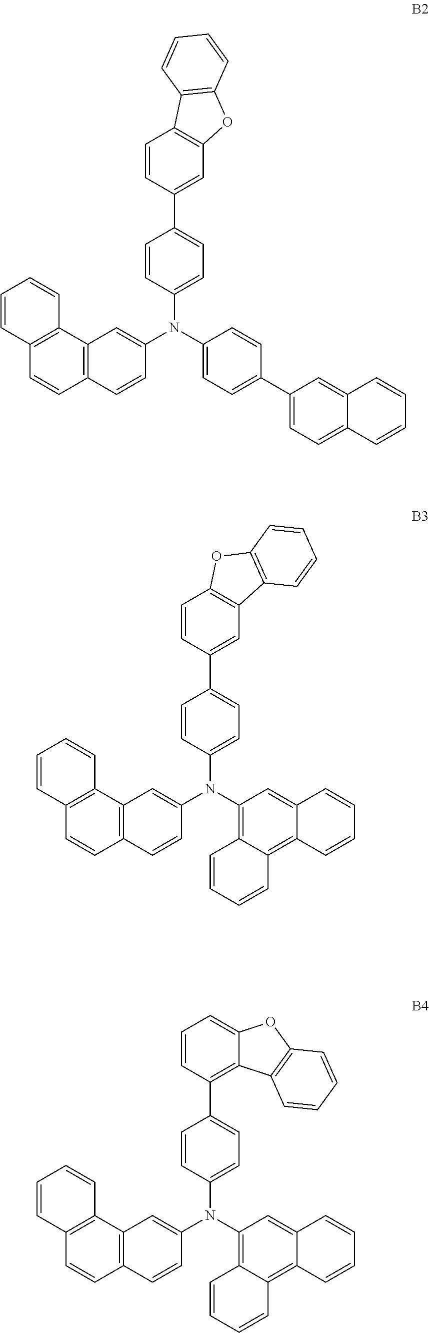 [SMILES: c1ccc2cc(-c3ccc(N(c4ccc(-c5ccc6c(c5)oc5ccccc56)cc4)c4ccc5ccc6ccccc6c5c4)cc3)ccc2c1 ; c1ccc2c(c1)ccc1ccc(N(c3ccc(-c4ccc5oc6ccccc6c5c4)cc3)c3cc4ccccc4c4ccccc34)cc12 ; c1ccc2c(c1)ccc1ccc(N(c3ccc(-c4cccc5oc6ccccc6c45)cc3)c3cc4ccccc4c4ccccc34)cc12]

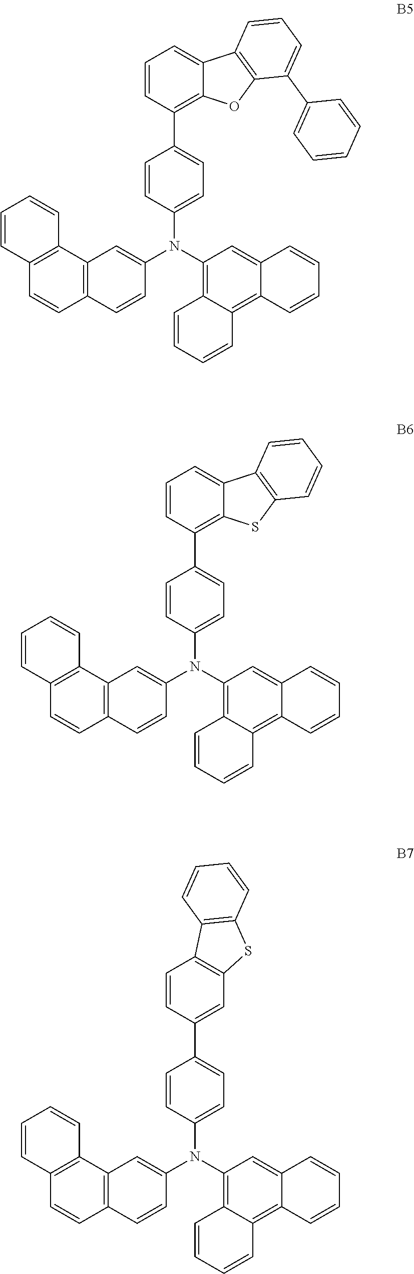 [SMILES: c1ccc(-c2cccc3c2oc2c(-c4ccc(N(c5ccc6ccc7ccccc7c6c5)c5cc6ccccc6c6ccccc56)cc4)cccc23)cc1 ; c1ccc2c(c1)ccc1ccc(N(c3ccc(-c4cccc5c4sc4ccccc45)cc3)c3cc4ccccc4c4ccccc34)cc12 ; c1ccc2c(c1)ccc1ccc(N(c3ccc(-c4ccc5c(c4)sc4ccccc45)cc3)c3cc4ccccc4c4ccccc34)cc12]

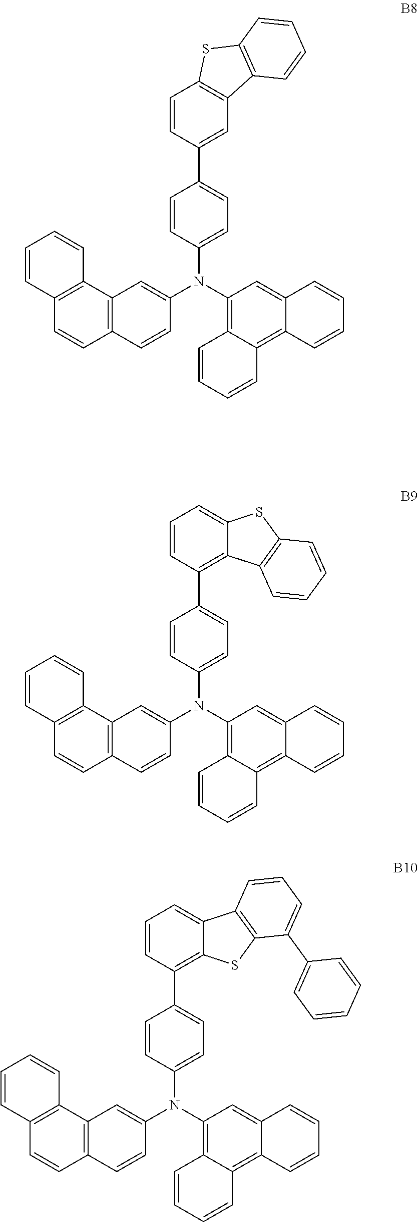 [SMILES: c1ccc2c(c1)ccc1ccc(N(c3ccc(-c4ccc5sc6ccccc6c5c4)cc3)c3cc4ccccc4c4ccccc34)cc12 ; c1ccc2c(c1)ccc1ccc(N(c3ccc(-c4cccc5sc6ccccc6c45)cc3)c3cc4ccccc4c4ccccc34)cc12 ; c1ccc(-c2cccc3c2sc2c(-c4ccc(N(c5ccc6ccc7ccccc7c6c5)c5cc6ccccc6c6ccccc56)cc4)cccc23)cc1]

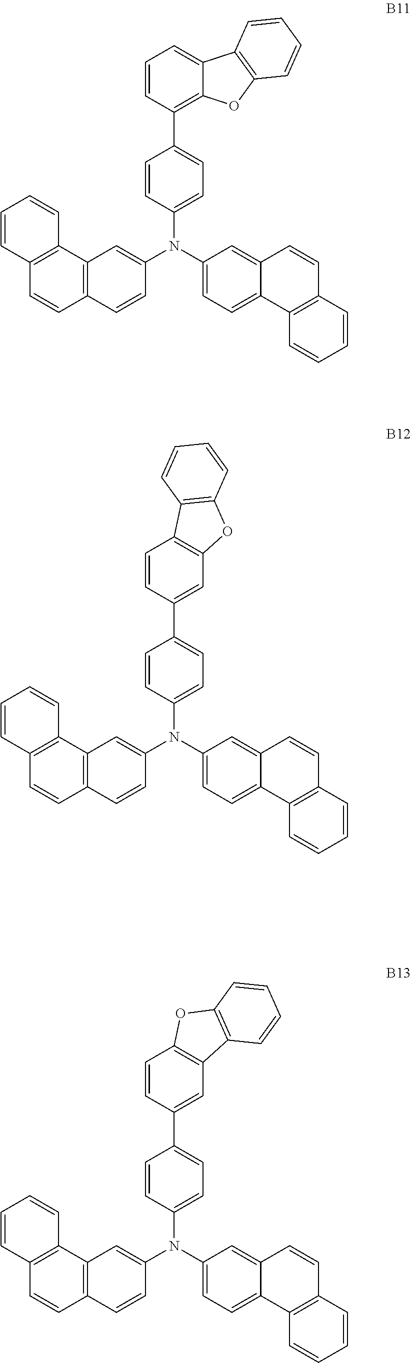 [SMILES: c1ccc2c(c1)ccc1cc(N(c3ccc(-c4cccc5c4oc4ccccc45)cc3)c3ccc4ccc5ccccc5c4c3)ccc12 ; c1ccc2c(c1)ccc1cc(N(c3ccc(-c4ccc5c(c4)oc4ccccc45)cc3)c3ccc4ccc5ccccc5c4c3)ccc12 ; c1ccc2c(c1)ccc1cc(N(c3ccc(-c4ccc5oc6ccccc6c5c4)cc3)c3ccc4ccc5ccccc5c4c3)ccc12]

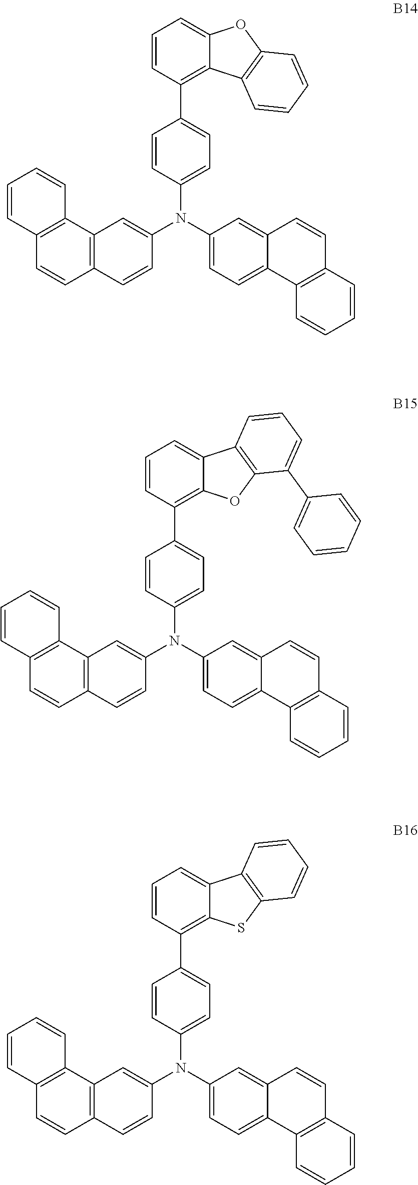 [SMILES: c1ccc2c(c1)ccc1cc(N(c3ccc(-c4cccc5oc6ccccc6c45)cc3)c3ccc4ccc5ccccc5c4c3)ccc12 ; c1ccc(-c2cccc3c2oc2c(-c4ccc(N(c5ccc6c(ccc7ccccc76)c5)c5ccc6ccc7ccccc7c6c5)cc4)cccc23)cc1 ; c1ccc2c(c1)ccc1cc(N(c3ccc(-c4cccc5c4sc4ccccc45)cc3)c3ccc4ccc5ccccc5c4c3)ccc12]

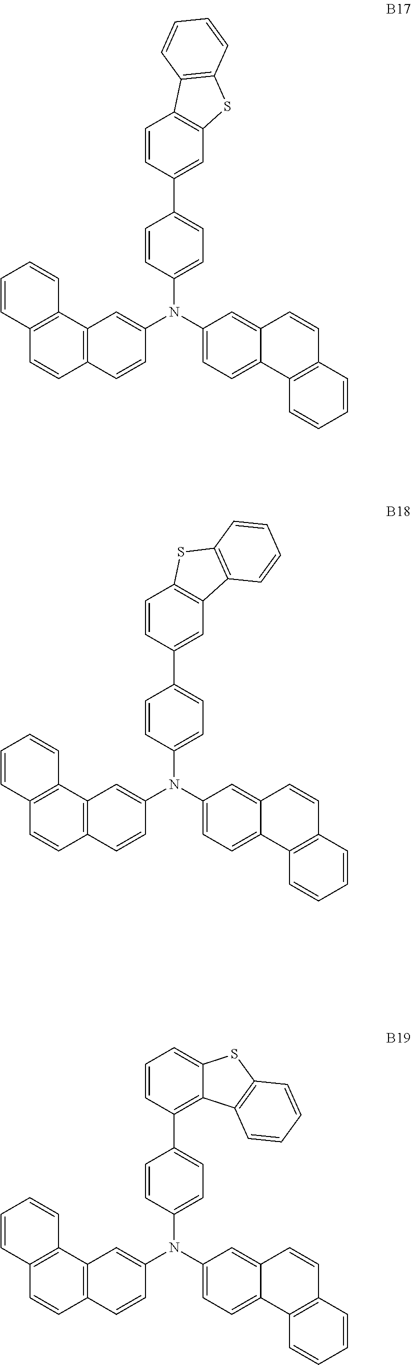 [SMILES: c1ccc2c(c1)ccc1cc(N(c3ccc(-c4ccc5c(c4)sc4ccccc45)cc3)c3ccc4ccc5ccccc5c4c3)ccc12 ; c1ccc2c(c1)ccc1cc(N(c3ccc(-c4ccc5sc6ccccc6c5c4)cc3)c3ccc4ccc5ccccc5c4c3)ccc12 ; c1ccc2c(c1)ccc1cc(N(c3ccc(-c4cccc5sc6ccccc6c45)cc3)c3ccc4ccc5ccccc5c4c3)ccc12]

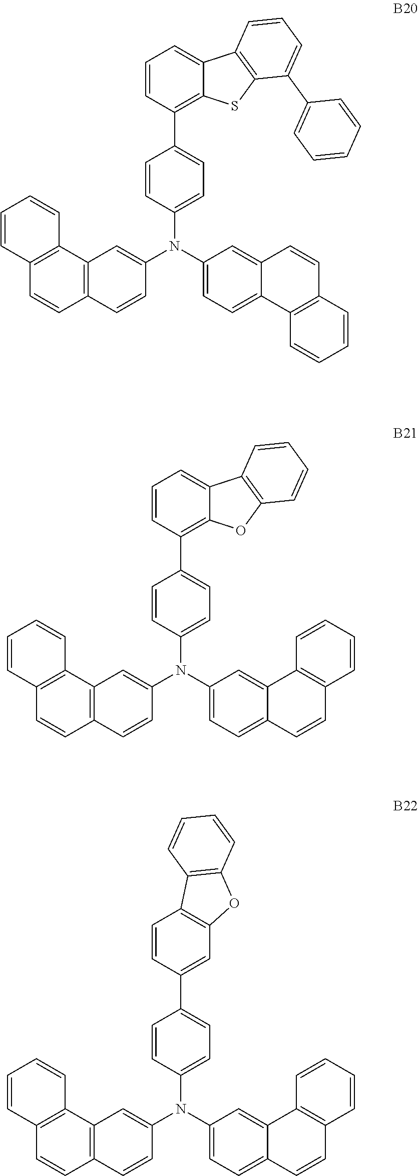 [SMILES: c1ccc(-c2cccc3c2sc2c(-c4ccc(N(c5ccc6c(ccc7ccccc76)c5)c5ccc6ccc7ccccc7c6c5)cc4)cccc23)cc1 ; c1ccc2c(c1)ccc1ccc(N(c3ccc(-c4cccc5c4oc4ccccc45)cc3)c3ccc4ccc5ccccc5c4c3)cc12 ; c1ccc2c(c1)ccc1ccc(N(c3ccc(-c4ccc5c(c4)oc4ccccc45)cc3)c3ccc4ccc5ccccc5c4c3)cc12]

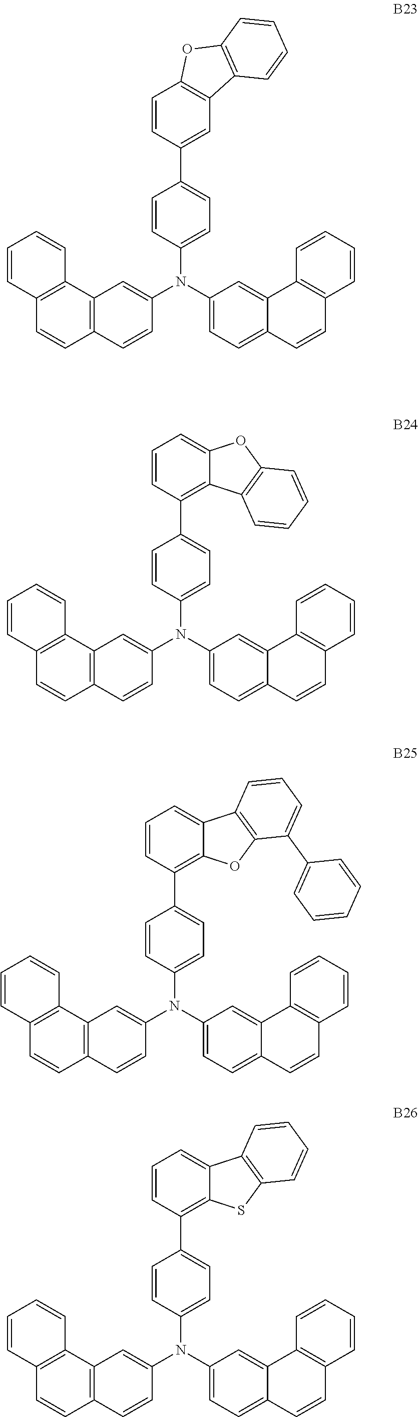 [SMILES: c1ccc2c(c1)ccc1ccc(N(c3ccc(-c4ccc5oc6ccccc6c5c4)cc3)c3ccc4ccc5ccccc5c4c3)cc12 ; c1ccc2c(c1)ccc1ccc(N(c3ccc(-c4cccc5oc6ccccc6c45)cc3)c3ccc4ccc5ccccc5c4c3)cc12 ; c1ccc(-c2cccc3c2oc2c(-c4ccc(N(c5ccc6ccc7ccccc7c6c5)c5ccc6ccc7ccccc7c6c5)cc4)cccc23)cc1 ; c1ccc2c(c1)ccc1ccc(N(c3ccc(-c4cccc5c4sc4ccccc45)cc3)c3ccc4ccc5ccccc5c4c3)cc12]

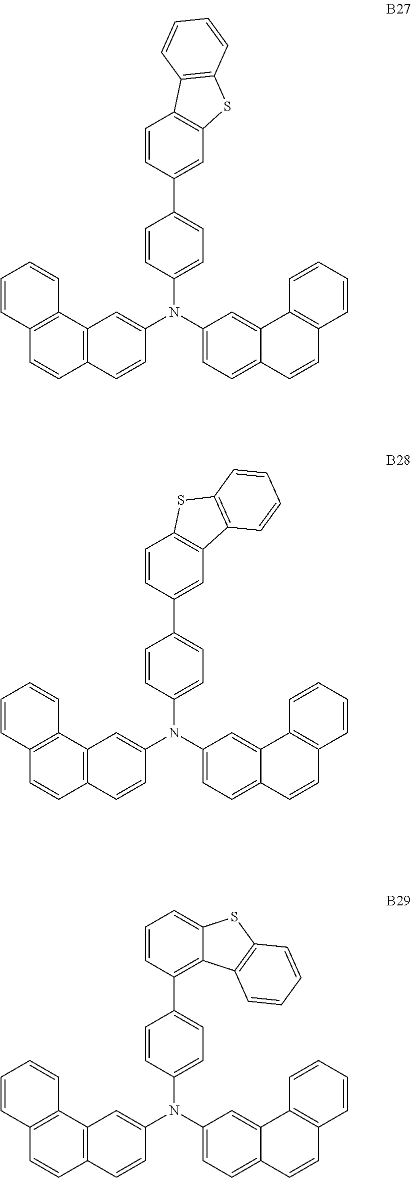 [SMILES: c1ccc2c(c1)ccc1ccc(N(c3ccc(-c4ccc5c(c4)sc4ccccc45)cc3)c3ccc4ccc5ccccc5c4c3)cc12 ; c1ccc2c(c1)ccc1ccc(N(c3ccc(-c4ccc5sc6ccccc6c5c4)cc3)c3ccc4ccc5ccccc5c4c3)cc12 ; c1ccc2c(c1)ccc1ccc(N(c3ccc(-c4cccc5sc6ccccc6c45)cc3)c3ccc4ccc5ccccc5c4c3)cc12]

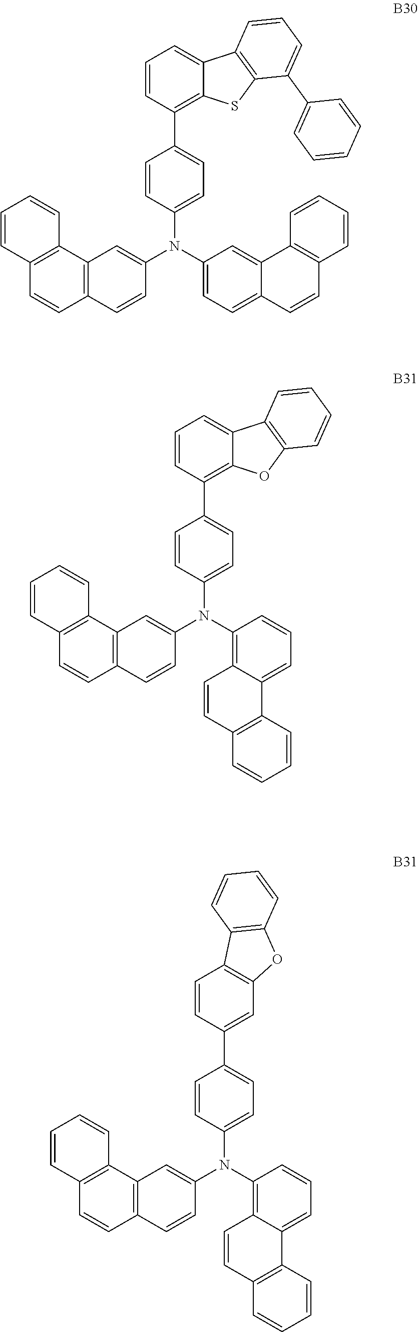 [SMILES: c1ccc(-c2cccc3c2sc2c(-c4ccc(N(c5ccc6ccc7ccccc7c6c5)c5ccc6ccc7ccccc7c6c5)cc4)cccc23)cc1 ; c1ccc2c(c1)ccc1ccc(N(c3ccc(-c4cccc5c4oc4ccccc45)cc3)c3cccc4c3ccc3ccccc34)cc12 ; c1ccc2c(c1)ccc1ccc(N(c3ccc(-c4ccc5c(c4)oc4ccccc45)cc3)c3cccc4c3ccc3ccccc34)cc12]

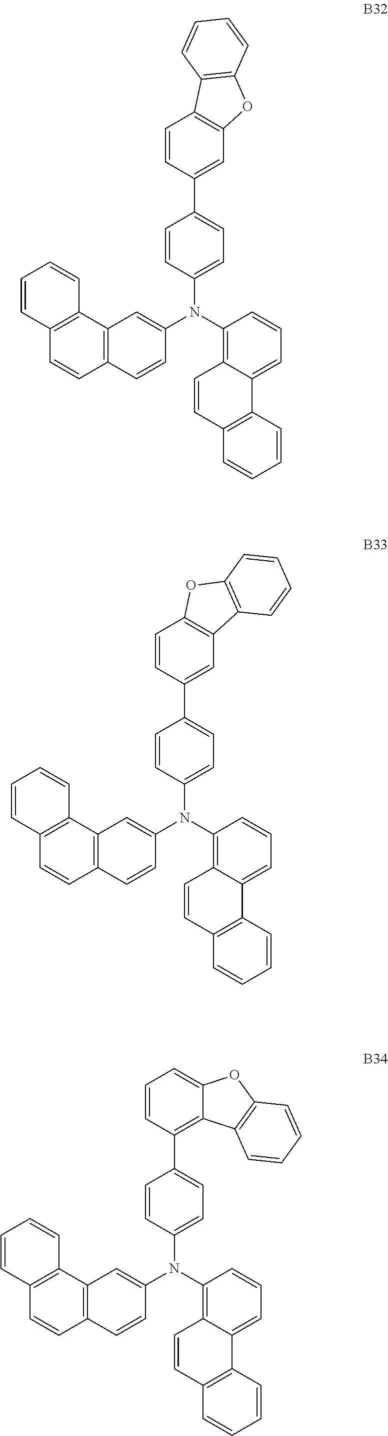 [SMILES: c1ccc2c(c1)ccc1ccc(N(c3ccc(-c4ccc5c(c4)oc4ccccc45)cc3)c3cccc4c3ccc3ccccc34)cc12 ; c1ccc2c(c1)ccc1ccc(N(c3ccc(-c4ccc5oc6ccccc6c5c4)cc3)c3cccc4c3ccc3ccccc34)cc12 ; c1ccc2c(c1)ccc1ccc(N(c3ccc(-c4cccc5oc6ccccc6c45)cc3)c3cccc4c3ccc3ccccc34)cc12]

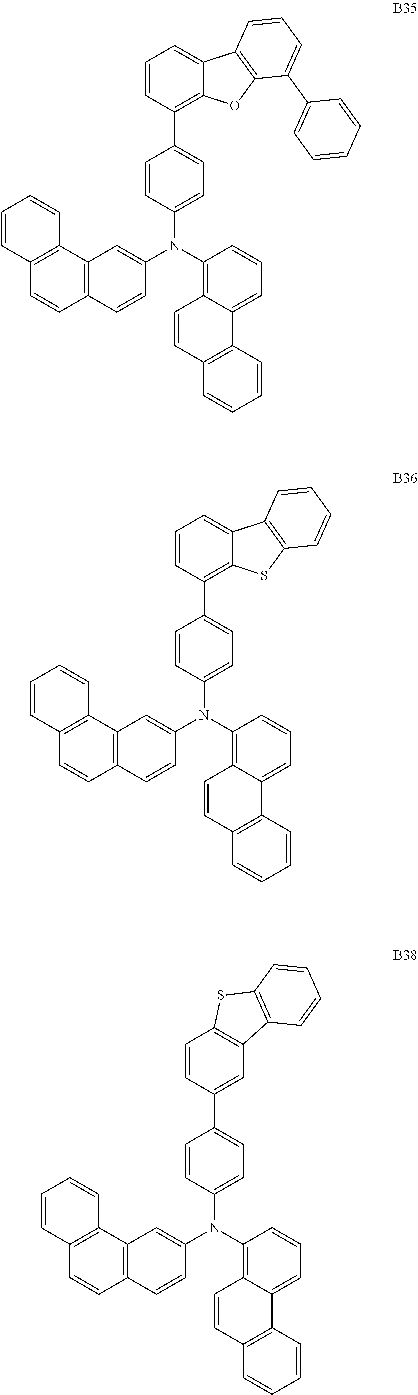 [SMILES: c1ccc(-c2cccc3c2oc2c(-c4ccc(N(c5ccc6ccc7ccccc7c6c5)c5cccc6c5ccc5ccccc56)cc4)cccc23)cc1 ; c1ccc2c(c1)ccc1ccc(N(c3ccc(-c4cccc5c4sc4ccccc45)cc3)c3cccc4c3ccc3ccccc34)cc12 ; c1ccc2c(c1)ccc1ccc(N(c3ccc(-c4ccc5sc6ccccc6c5c4)cc3)c3cccc4c3ccc3ccccc34)cc12]

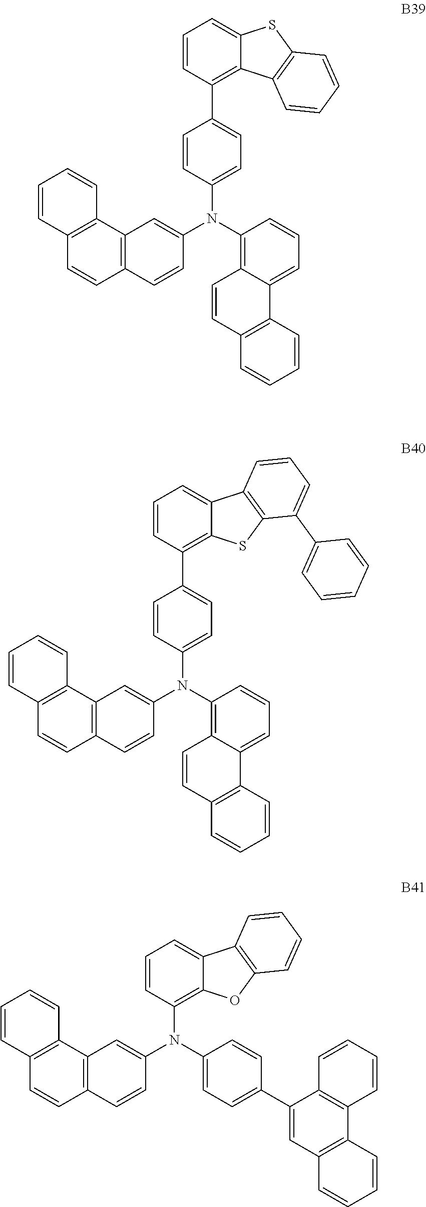 [SMILES: c1ccc2c(c1)ccc1ccc(N(c3ccc(-c4cccc5sc6ccccc6c45)cc3)c3cccc4c3ccc3ccccc34)cc12 ; c1ccc(-c2cccc3c2sc2c(-c4ccc(N(c5ccc6ccc7ccccc7c6c5)c5cccc6c5ccc5ccccc56)cc4)cccc23)cc1 ; c1ccc2c(c1)ccc1ccc(N(c3ccc(-c4cc5ccccc5c5ccccc45)cc3)c3cccc4c3oc3ccccc34)cc12]

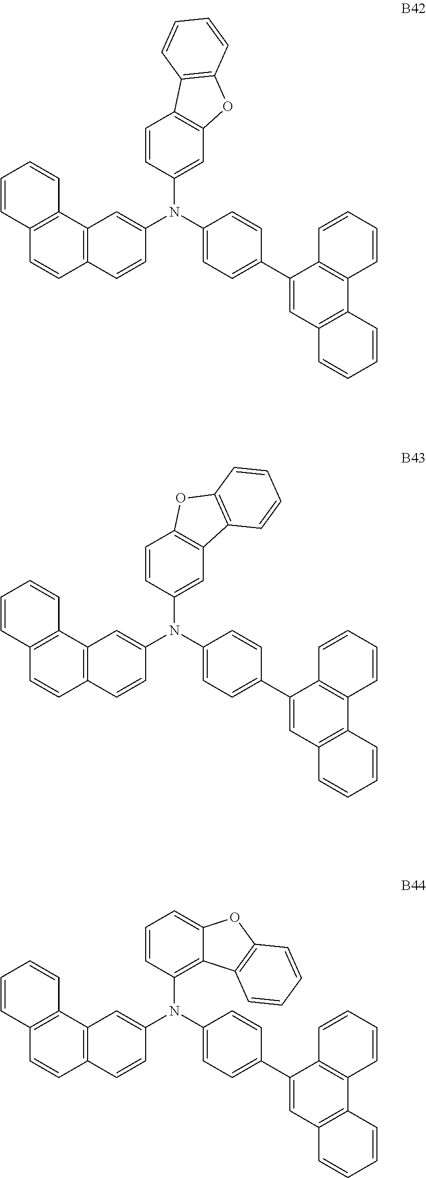 [SMILES: c1ccc2c(c1)ccc1ccc(N(c3ccc(-c4cc5ccccc5c5ccccc45)cc3)c3ccc4c(c3)oc3ccccc34)cc12 ; c1ccc2c(c1)ccc1ccc(N(c3ccc(-c4cc5ccccc5c5ccccc45)cc3)c3ccc4oc5ccccc5c4c3)cc12 ; c1ccc2c(c1)ccc1ccc(N(c3ccc(-c4cc5ccccc5c5ccccc45)cc3)c3cccc4oc5ccccc5c34)cc12]

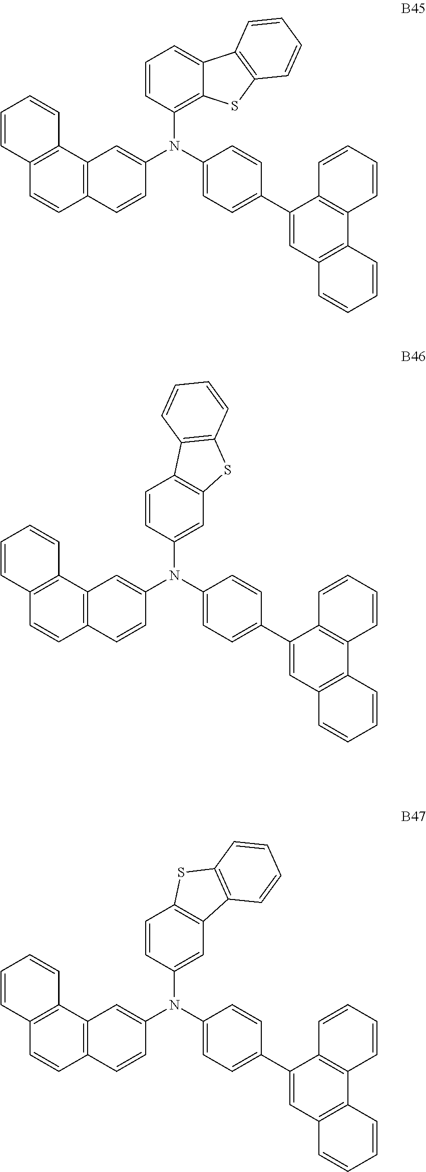 [SMILES: c1ccc2c(c1)ccc1ccc(N(c3ccc(-c4cc5ccccc5c5ccccc45)cc3)c3cccc4c3sc3ccccc34)cc12 ; c1ccc2c(c1)ccc1ccc(N(c3ccc(-c4cc5ccccc5c5ccccc45)cc3)c3ccc4c(c3)sc3ccccc34)cc12 ; c1ccc2c(c1)ccc1ccc(N(c3ccc(-c4cc5ccccc5c5ccccc45)cc3)c3ccc4sc5ccccc5c4c3)cc12]

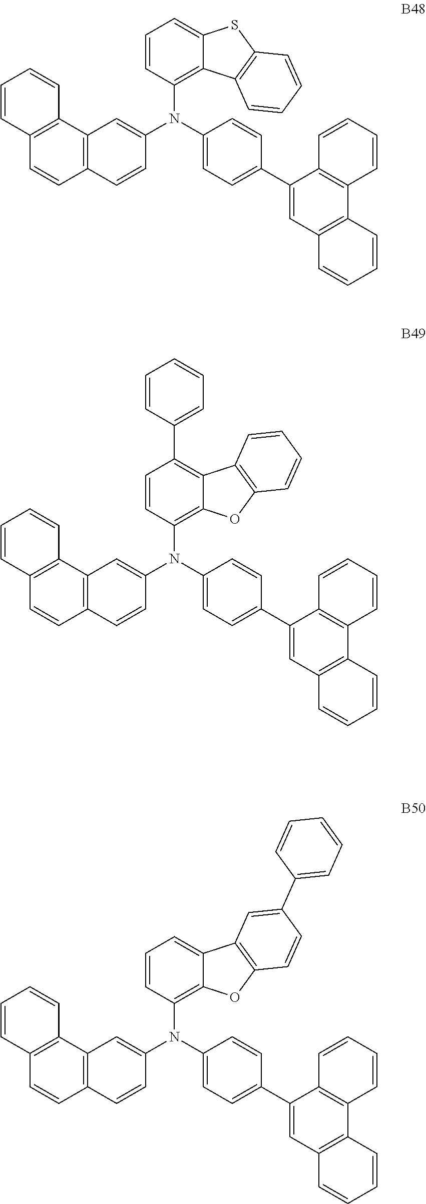 [SMILES: c1ccc2c(c1)ccc1ccc(N(c3ccc(-c4cc5ccccc5c5ccccc45)cc3)c3cccc4sc5ccccc5c34)cc12 ; c1ccc(-c2ccc(N(c3ccc(-c4cc5ccccc5c5ccccc45)cc3)c3ccc4ccc5ccccc5c4c3)c3oc4ccccc4c23)cc1 ; c1ccc(-c2ccc3oc4c(N(c5ccc(-c6cc7ccccc7c7ccccc67)cc5)c5ccc6ccc7ccccc7c6c5)cccc4c3c2)cc1]

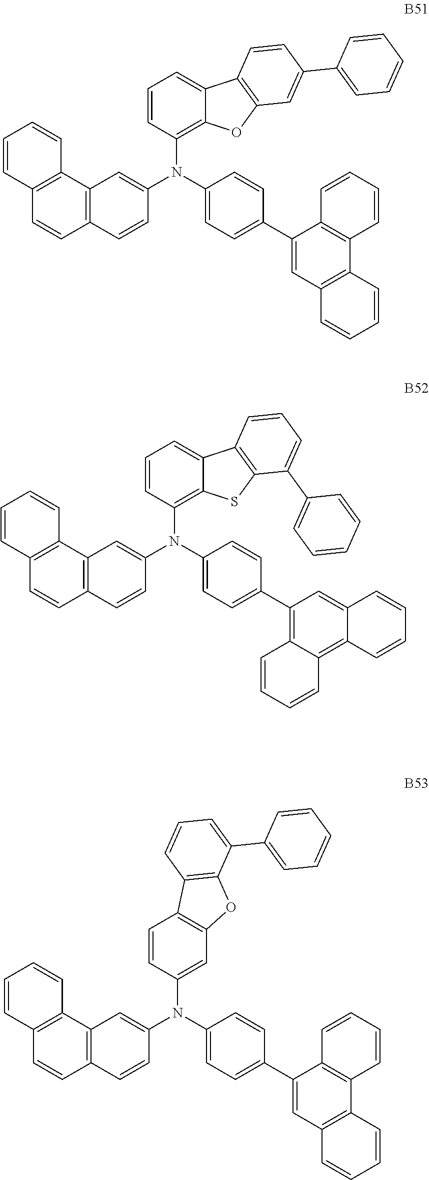 [SMILES: c1ccc(-c2ccc3c(c2)oc2c(N(c4ccc(-c5cc6ccccc6c6ccccc56)cc4)c4ccc5ccc6ccccc6c5c4)cccc23)cc1 ; c1ccc(-c2cccc3c2sc2c(N(c4ccc(-c5cc6ccccc6c6ccccc56)cc4)c4ccc5ccc6ccccc6c5c4)cccc23)cc1 ; c1ccc(-c2cccc3c2oc2cc(N(c4ccc(-c5cc6ccccc6c6ccccc56)cc4)c4ccc5ccc6ccccc6c5c4)ccc23)cc1]

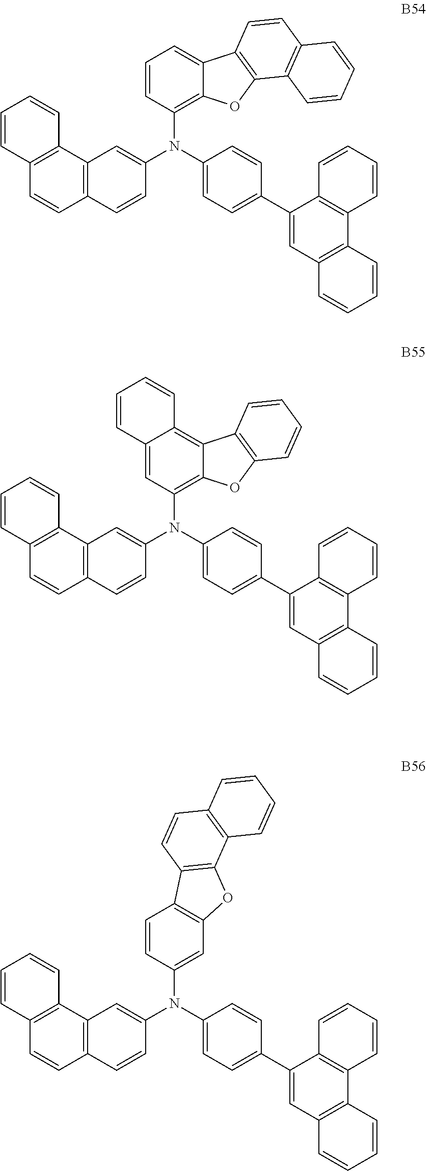 [SMILES: c1ccc2c(c1)ccc1ccc(N(c3ccc(-c4cc5ccccc5c5ccccc45)cc3)c3cccc4c3oc3c5ccccc5ccc43)cc12 ; c1ccc2c(c1)ccc1ccc(N(c3ccc(-c4cc5ccccc5c5ccccc45)cc3)c3cc4ccccc4c4c3oc3ccccc34)cc12 ; c1ccc2c(c1)ccc1ccc(N(c3ccc(-c4cc5ccccc5c5ccccc45)cc3)c3ccc4c(c3)oc3c5ccccc5ccc43)cc12]

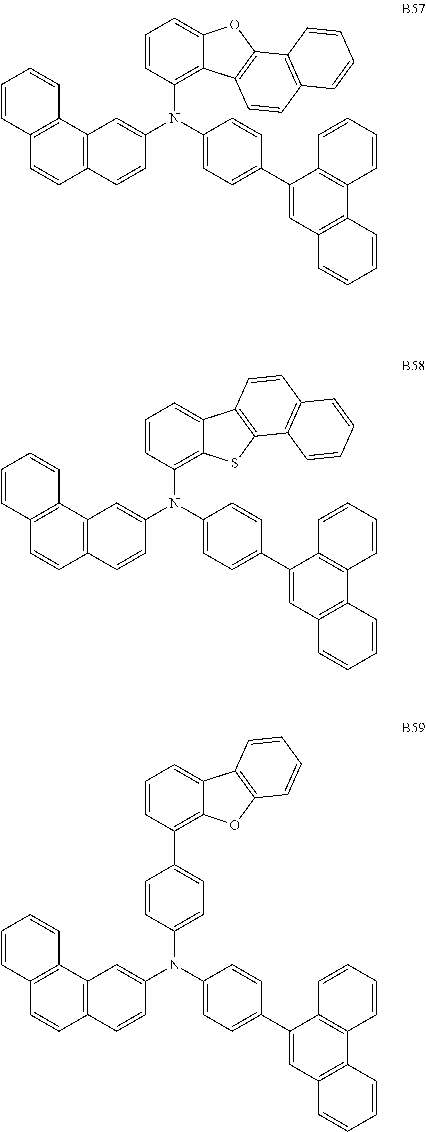 [SMILES: c1ccc2c(c1)ccc1ccc(N(c3ccc(-c4cc5ccccc5c5ccccc45)cc3)c3cccc4oc5c6ccccc6ccc5c34)cc12 ; c1ccc2c(c1)ccc1ccc(N(c3ccc(-c4cc5ccccc5c5ccccc45)cc3)c3cccc4c3sc3c5ccccc5ccc43)cc12 ; c1ccc2c(c1)ccc1ccc(N(c3ccc(-c4cc5ccccc5c5ccccc45)cc3)c3ccc(-c4cccc5c4oc4ccccc45)cc3)cc12]

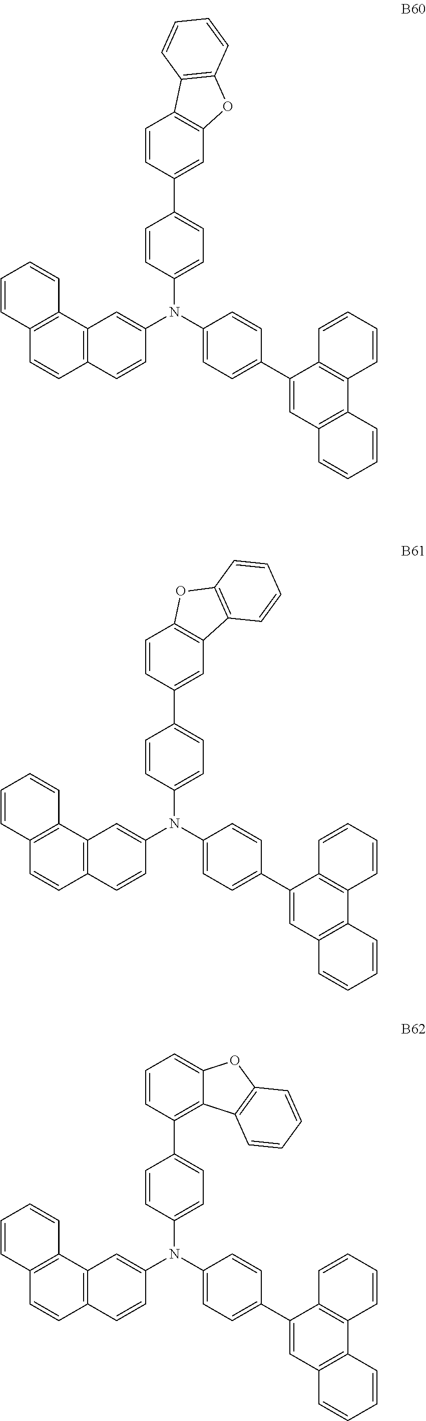 [SMILES: c1ccc2c(c1)ccc1ccc(N(c3ccc(-c4ccc5c(c4)oc4ccccc45)cc3)c3ccc(-c4cc5ccccc5c5ccccc45)cc3)cc12 ; c1ccc2c(c1)ccc1ccc(N(c3ccc(-c4ccc5oc6ccccc6c5c4)cc3)c3ccc(-c4cc5ccccc5c5ccccc45)cc3)cc12 ; c1ccc2c(c1)ccc1ccc(N(c3ccc(-c4cc5ccccc5c5ccccc45)cc3)c3ccc(-c4cccc5oc6ccccc6c45)cc3)cc12]

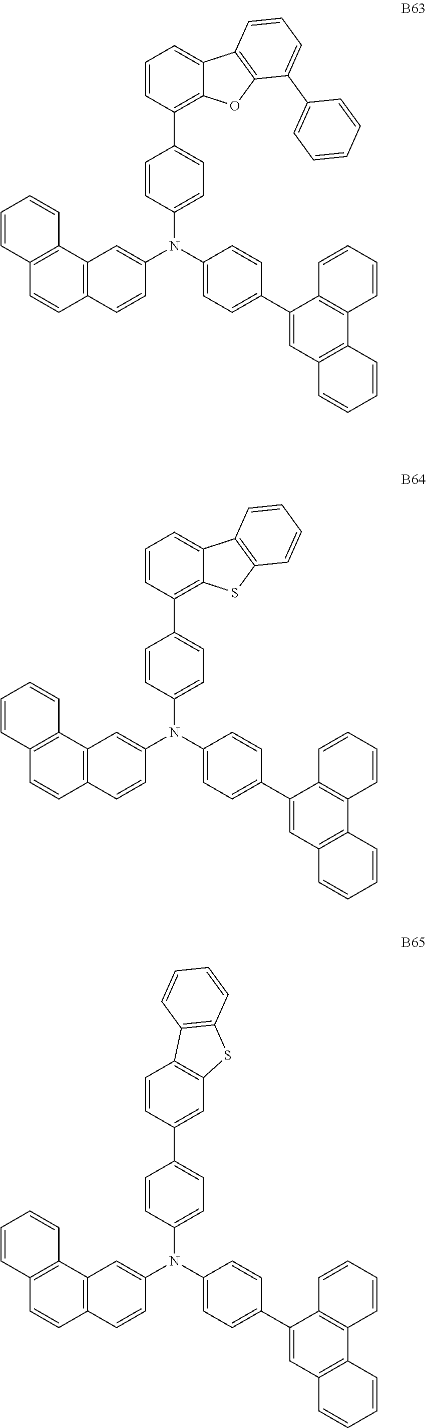 [SMILES: c1ccc(-c2cccc3c2oc2c(-c4ccc(N(c5ccc(-c6cc7ccccc7c7ccccc67)cc5)c5ccc6ccc7ccccc7c6c5)cc4)cccc23)cc1 ; c1ccc2c(c1)ccc1ccc(N(c3ccc(-c4cc5ccccc5c5ccccc45)cc3)c3ccc(-c4cccc5c4sc4ccccc45)cc3)cc12 ; c1ccc2c(c1)ccc1ccc(N(c3ccc(-c4ccc5c(c4)sc4ccccc45)cc3)c3ccc(-c4cc5ccccc5c5ccccc45)cc3)cc12]

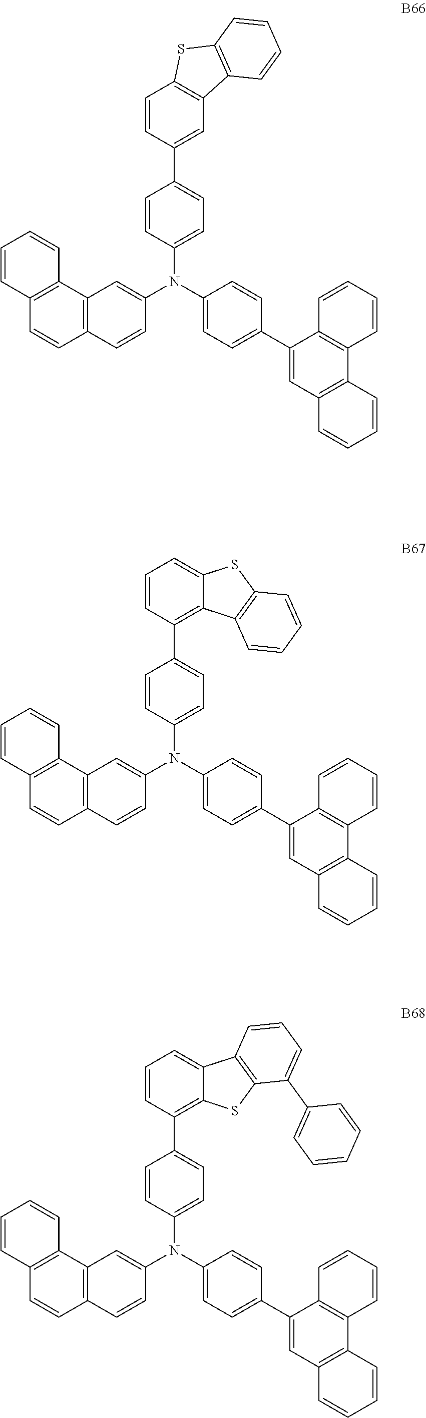 [SMILES: c1ccc2c(c1)ccc1ccc(N(c3ccc(-c4ccc5sc6ccccc6c5c4)cc3)c3ccc(-c4cc5ccccc5c5ccccc45)cc3)cc12 ; c1ccc2c(c1)ccc1ccc(N(c3ccc(-c4cc5ccccc5c5ccccc45)cc3)c3ccc(-c4cccc5sc6ccccc6c45)cc3)cc12 ; c1ccc(-c2cccc3c2sc2c(-c4ccc(N(c5ccc(-c6cc7ccccc7c7ccccc67)cc5)c5ccc6ccc7ccccc7c6c5)cc4)cccc23)cc1]

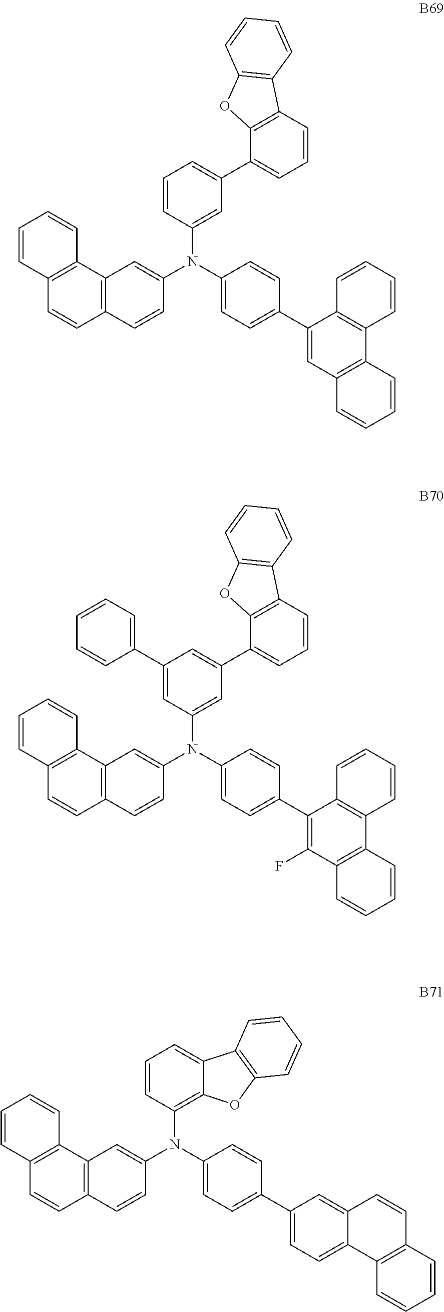 [SMILES: c1cc(-c2cccc3c2oc2ccccc23)cc(N(c2ccc(-c3cc4ccccc4c4ccccc34)cc2)c2ccc3ccc4ccccc4c3c2)c1 ; Fc1c(-c2ccc(N(c3cc(-c4ccccc4)cc(-c4cccc5c4oc4ccccc45)c3)c3ccc4ccc5ccccc5c4c3)cc2)c2ccccc2c2ccccc12 ; c1ccc2c(c1)ccc1cc(-c3ccc(N(c4ccc5ccc6ccccc6c5c4)c4cccc5c4oc4ccccc45)cc3)ccc12]

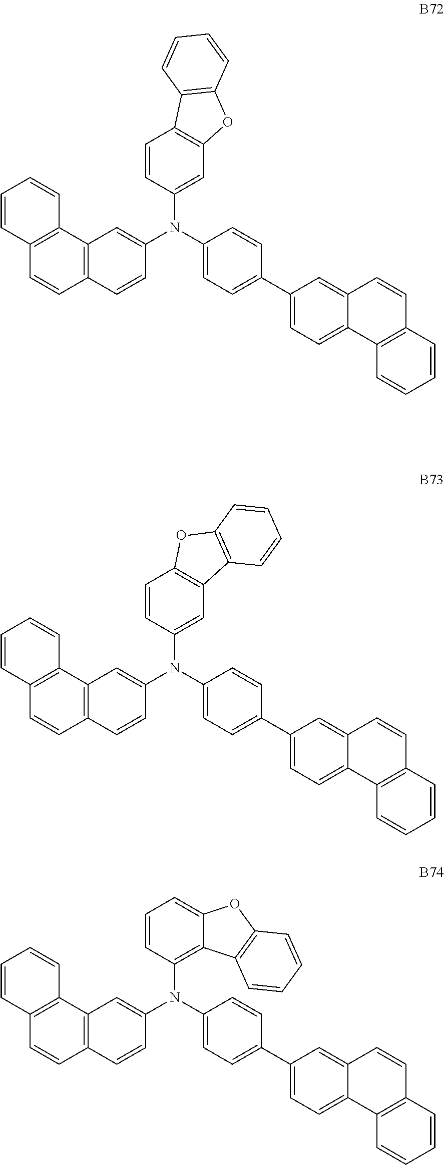 [SMILES: c1ccc2c(c1)ccc1cc(-c3ccc(N(c4ccc5c(c4)oc4ccccc45)c4ccc5ccc6ccccc6c5c4)cc3)ccc12 ; c1ccc2c(c1)ccc1cc(-c3ccc(N(c4ccc5ccc6ccccc6c5c4)c4ccc5oc6ccccc6c5c4)cc3)ccc12 ; c1ccc2c(c1)ccc1cc(-c3ccc(N(c4ccc5ccc6ccccc6c5c4)c4cccc5oc6ccccc6c45)cc3)ccc12]

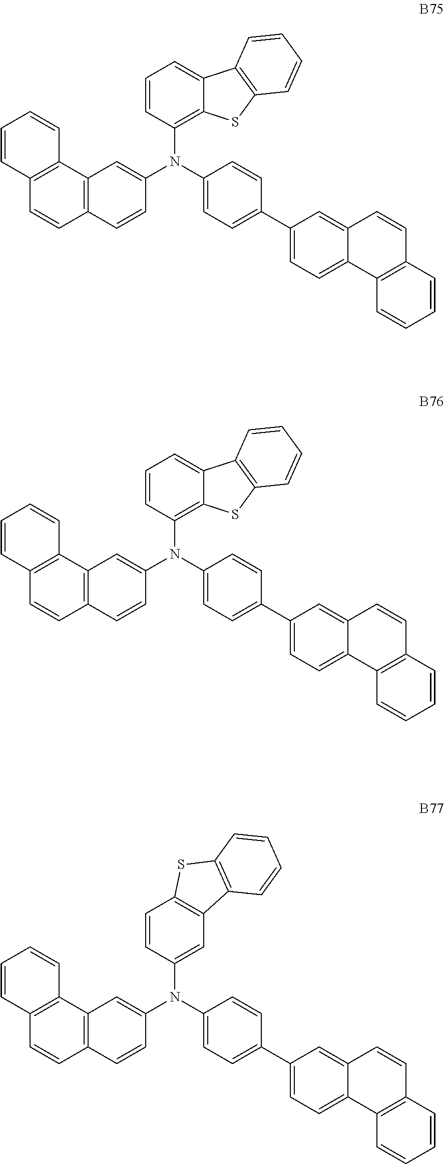 [SMILES: c1ccc2c(c1)ccc1cc(-c3ccc(N(c4ccc5ccc6ccccc6c5c4)c4cccc5c4sc4ccccc45)cc3)ccc12 ; c1ccc2c(c1)ccc1cc(-c3ccc(N(c4ccc5ccc6ccccc6c5c4)c4cccc5c4sc4ccccc45)cc3)ccc12 ; c1ccc2c(c1)ccc1cc(-c3ccc(N(c4ccc5ccc6ccccc6c5c4)c4ccc5sc6ccccc6c5c4)cc3)ccc12]

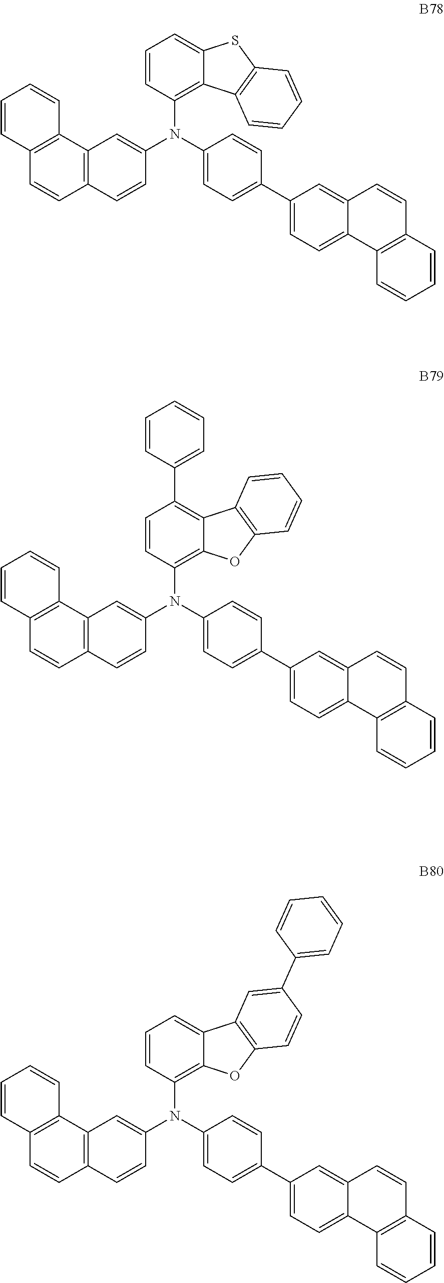 [SMILES: c1ccc2c(c1)ccc1cc(-c3ccc(N(c4ccc5ccc6ccccc6c5c4)c4cccc5sc6ccccc6c45)cc3)ccc12 ; c1ccc(-c2ccc(N(c3ccc(-c4ccc5c(ccc6ccccc65)c4)cc3)c3ccc4ccc5ccccc5c4c3)c3oc4ccccc4c23)cc1 ; c1ccc(-c2ccc3oc4c(N(c5ccc(-c6ccc7c(ccc8ccccc87)c6)cc5)c5ccc6ccc7ccccc7c6c5)cccc4c3c2)cc1]

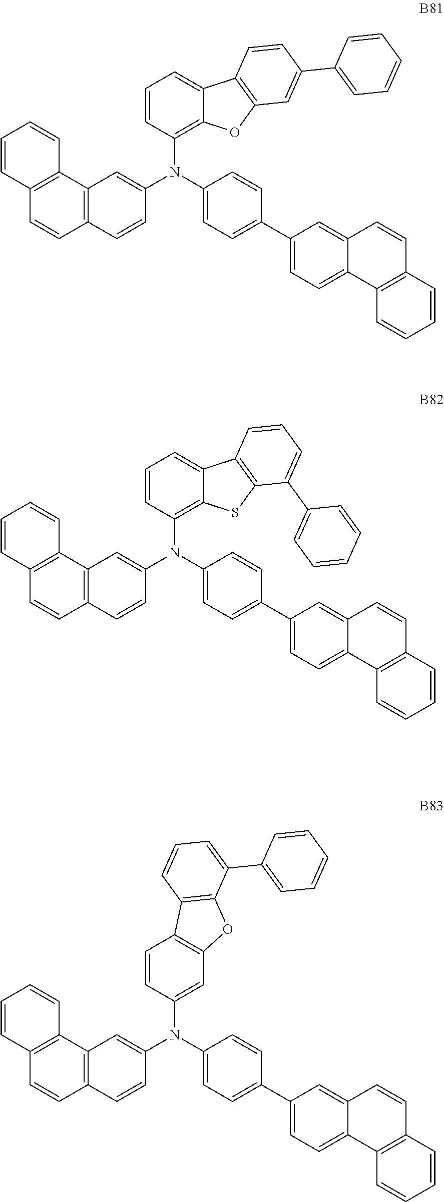 [SMILES: c1ccc(-c2ccc3c(c2)oc2c(N(c4ccc(-c5ccc6c(ccc7ccccc76)c5)cc4)c4ccc5ccc6ccccc6c5c4)cccc23)cc1 ; c1ccc(-c2cccc3c2sc2c(N(c4ccc(-c5ccc6c(ccc7ccccc76)c5)cc4)c4ccc5ccc6ccccc6c5c4)cccc23)cc1 ; c1ccc(-c2cccc3c2oc2cc(N(c4ccc(-c5ccc6c(ccc7ccccc76)c5)cc4)c4ccc5ccc6ccccc6c5c4)ccc23)cc1]

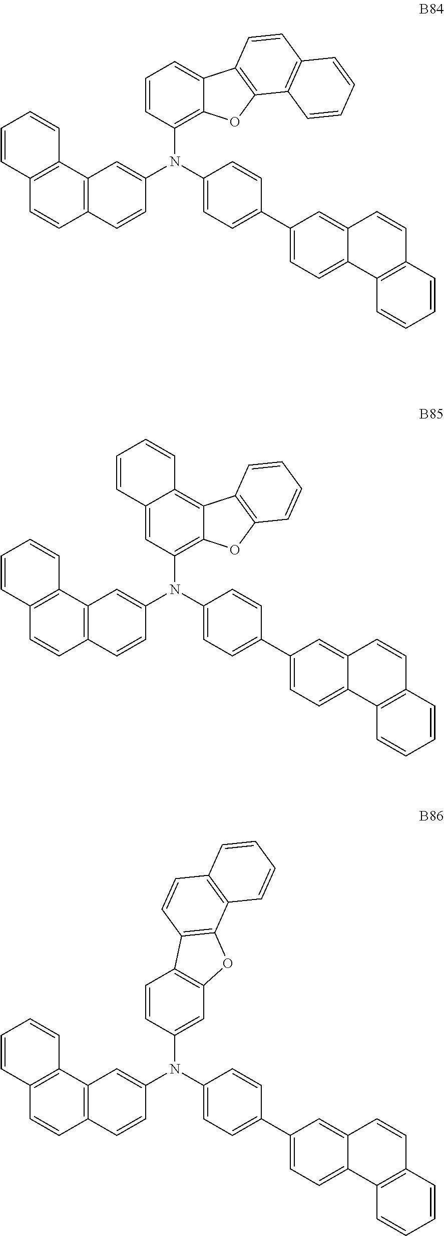 [SMILES: c1ccc2c(c1)ccc1cc(-c3ccc(N(c4ccc5ccc6ccccc6c5c4)c4cccc5c4oc4c6ccccc6ccc54)cc3)ccc12 ; c1ccc2c(c1)ccc1cc(-c3ccc(N(c4ccc5ccc6ccccc6c5c4)c4cc5ccccc5c5c4oc4ccccc45)cc3)ccc12 ; c1ccc2c(c1)ccc1cc(-c3ccc(N(c4ccc5c(c4)oc4c6ccccc6ccc54)c4ccc5ccc6ccccc6c5c4)cc3)ccc12]

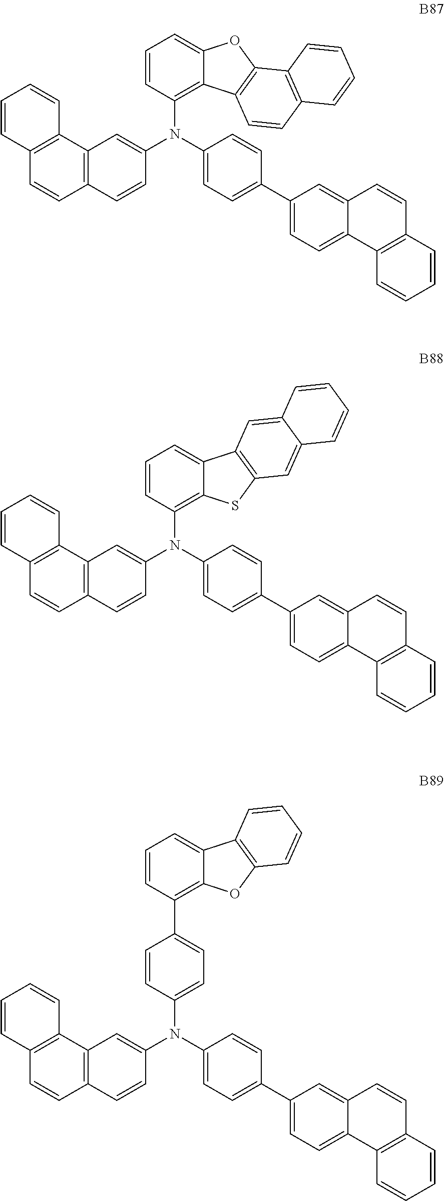 [SMILES: c1ccc2c(c1)ccc1cc(-c3ccc(N(c4ccc5ccc6ccccc6c5c4)c4cccc5oc6c7ccccc7ccc6c45)cc3)ccc12 ; c1ccc2cc3c(cc2c1)sc1c(N(c2ccc(-c4ccc5c(ccc6ccccc65)c4)cc2)c2ccc4ccc5ccccc5c4c2)cccc13 ; c1ccc2c(c1)ccc1cc(-c3ccc(N(c4ccc(-c5cccc6c5oc5ccccc56)cc4)c4ccc5ccc6ccccc6c5c4)cc3)ccc12]

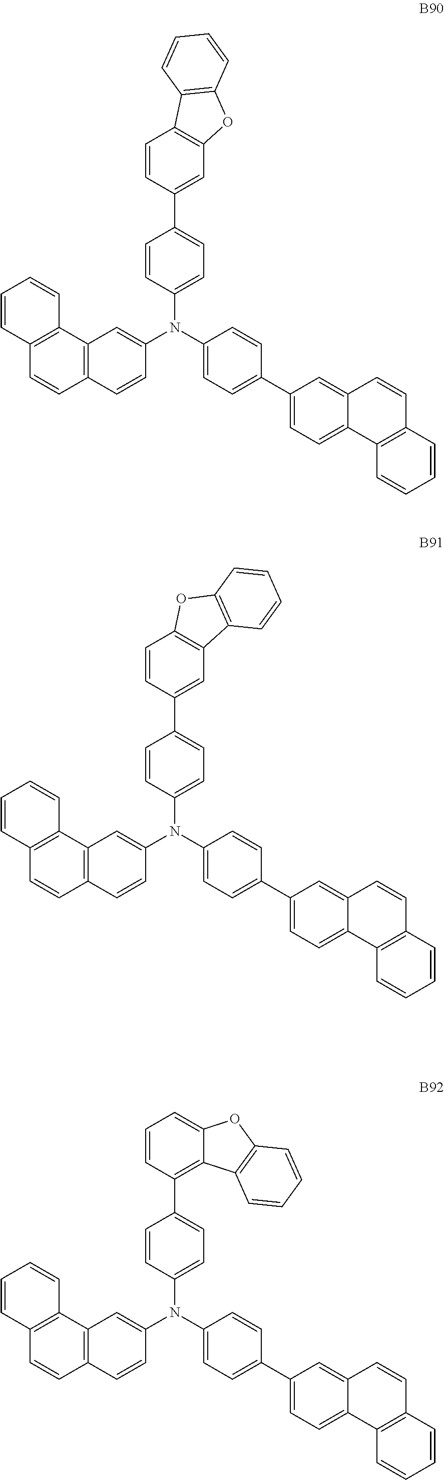 [SMILES: c1ccc2c(c1)ccc1cc(-c3ccc(N(c4ccc(-c5ccc6c(c5)oc5ccccc56)cc4)c4ccc5ccc6ccccc6c5c4)cc3)ccc12 ; c1ccc2c(c1)ccc1cc(-c3ccc(N(c4ccc(-c5ccc6oc7ccccc7c6c5)cc4)c4ccc5ccc6ccccc6c5c4)cc3)ccc12 ; c1ccc2c(c1)ccc1cc(-c3ccc(N(c4ccc(-c5cccc6oc7ccccc7c56)cc4)c4ccc5ccc6ccccc6c5c4)cc3)ccc12]

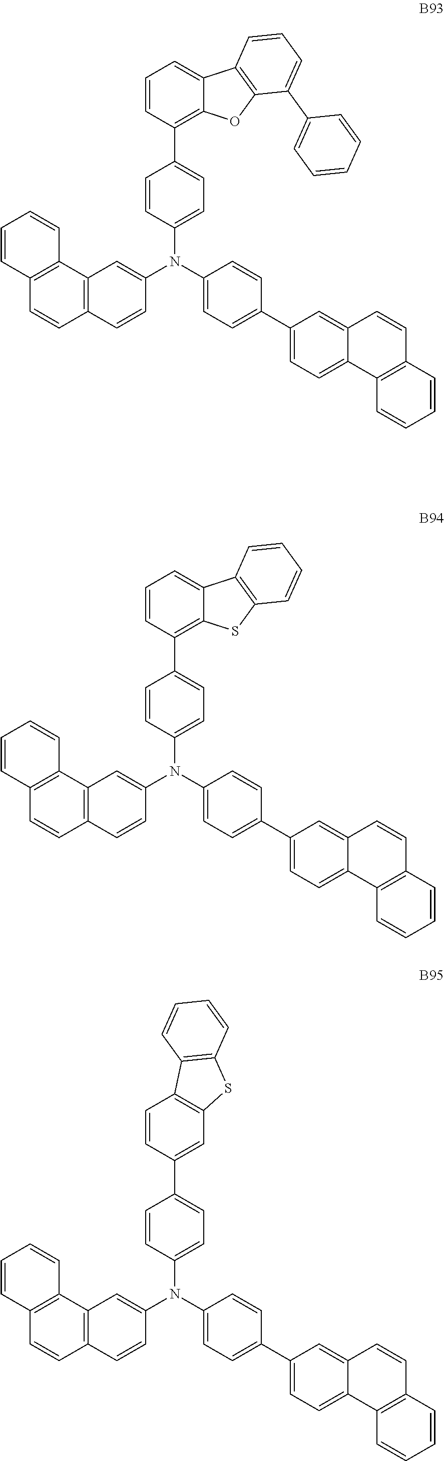 [SMILES: c1ccc(-c2cccc3c2oc2c(-c4ccc(N(c5ccc(-c6ccc7c(ccc8ccccc87)c6)cc5)c5ccc6ccc7ccccc7c6c5)cc4)cccc23)cc1 ; c1ccc2c(c1)ccc1cc(-c3ccc(N(c4ccc(-c5cccc6c5sc5ccccc56)cc4)c4ccc5ccc6ccccc6c5c4)cc3)ccc12 ; c1ccc2c(c1)ccc1cc(-c3ccc(N(c4ccc(-c5ccc6c(c5)sc5ccccc56)cc4)c4ccc5ccc6ccccc6c5c4)cc3)ccc12]

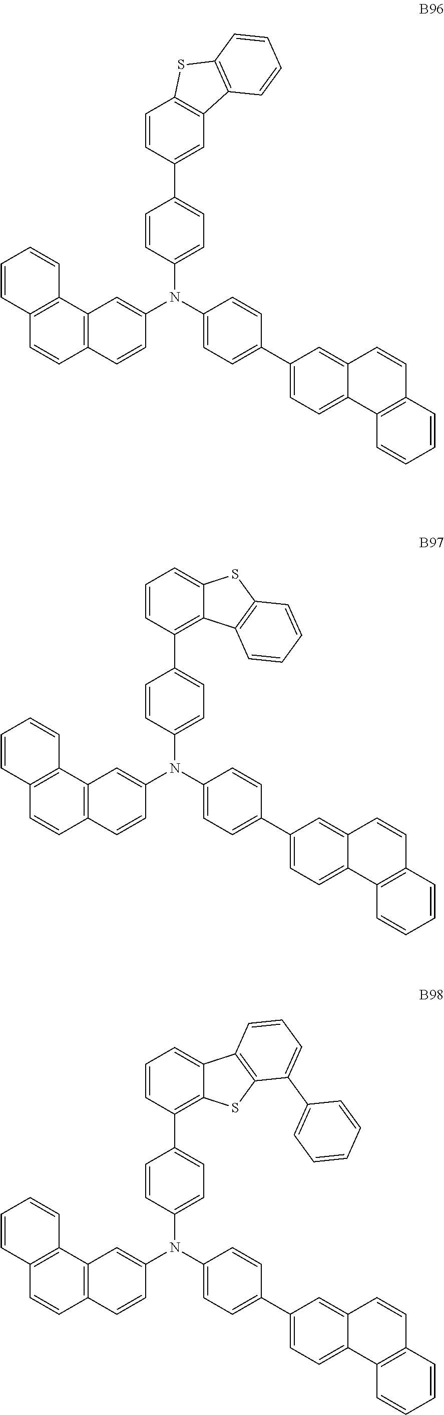 [SMILES: c1ccc2c(c1)ccc1cc(-c3ccc(N(c4ccc(-c5ccc6sc7ccccc7c6c5)cc4)c4ccc5ccc6ccccc6c5c4)cc3)ccc12 ; c1ccc2c(c1)ccc1cc(-c3ccc(N(c4ccc(-c5cccc6sc7ccccc7c56)cc4)c4ccc5ccc6ccccc6c5c4)cc3)ccc12 ; c1ccc(-c2cccc3c2sc2c(-c4ccc(N(c5ccc(-c6ccc7c(ccc8ccccc87)c6)cc5)c5ccc6ccc7ccccc7c6c5)cc4)cccc23)cc1]

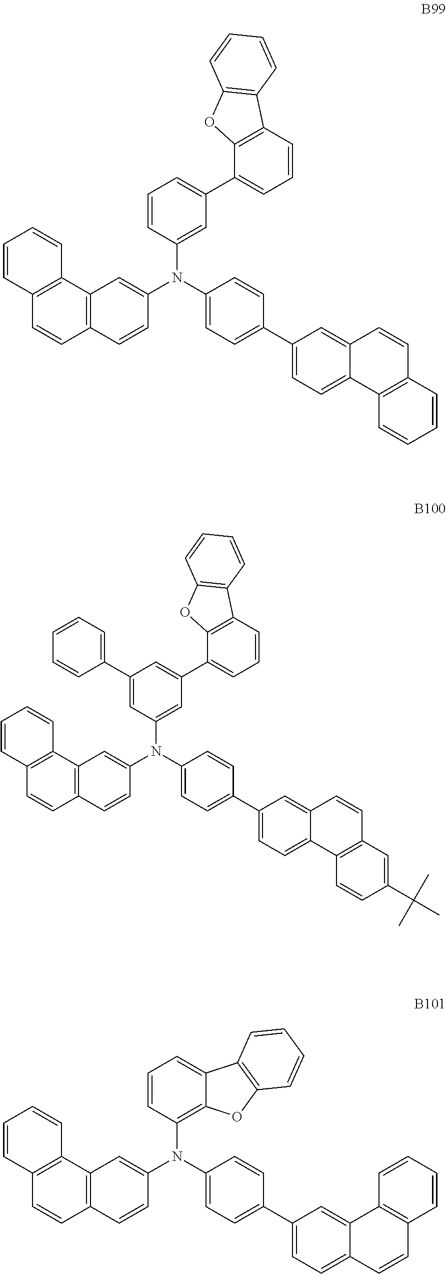 [SMILES: c1cc(-c2cccc3c2oc2ccccc23)cc(N(c2ccc(-c3ccc4c(ccc5ccccc54)c3)cc2)c2ccc3ccc4ccccc4c3c2)c1 ; CC(C)(C)c1ccc2c(ccc3cc(-c4ccc(N(c5cc(-c6ccccc6)cc(-c6cccc7c6oc6ccccc67)c5)c5ccc6ccc7ccccc7c6c5)cc4)ccc32)c1 ; c1ccc2c(c1)ccc1ccc(-c3ccc(N(c4ccc5ccc6ccccc6c5c4)c4cccc5c4oc4ccccc45)cc3)cc12]

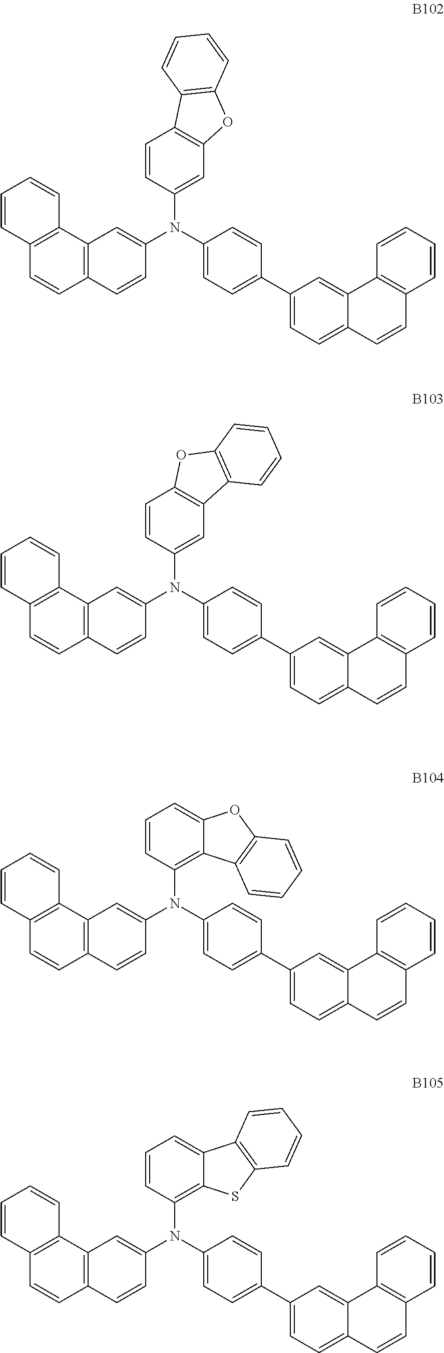 [SMILES: c1ccc2c(c1)ccc1ccc(-c3ccc(N(c4ccc5c(c4)oc4ccccc45)c4ccc5ccc6ccccc6c5c4)cc3)cc12 ; c1ccc2c(c1)ccc1ccc(-c3ccc(N(c4ccc5ccc6ccccc6c5c4)c4ccc5oc6ccccc6c5c4)cc3)cc12 ; c1ccc2c(c1)ccc1ccc(-c3ccc(N(c4ccc5ccc6ccccc6c5c4)c4cccc5oc6ccccc6c45)cc3)cc12 ; c1ccc2c(c1)ccc1ccc(-c3ccc(N(c4ccc5ccc6ccccc6c5c4)c4cccc5c4sc4ccccc45)cc3)cc12]

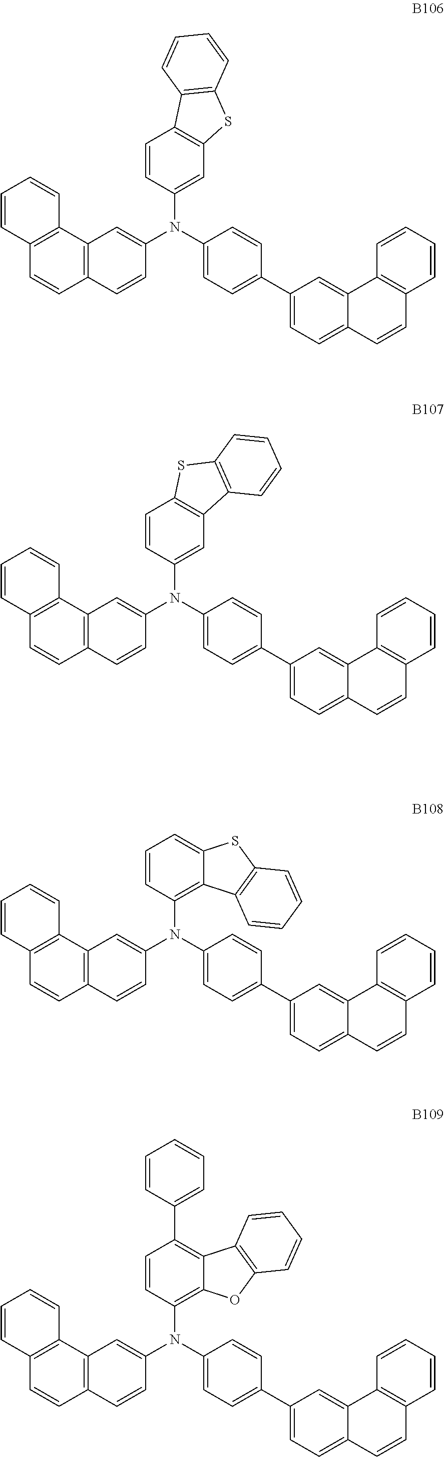 [SMILES: c1ccc2c(c1)ccc1ccc(-c3ccc(N(c4ccc5c(c4)sc4ccccc45)c4ccc5ccc6ccccc6c5c4)cc3)cc12 ; c1ccc2c(c1)ccc1ccc(-c3ccc(N(c4ccc5ccc6ccccc6c5c4)c4ccc5sc6ccccc6c5c4)cc3)cc12 ; c1ccc2c(c1)ccc1ccc(-c3ccc(N(c4ccc5ccc6ccccc6c5c4)c4cccc5sc6ccccc6c45)cc3)cc12 ; c1ccc(-c2ccc(N(c3ccc(-c4ccc5ccc6ccccc6c5c4)cc3)c3ccc4ccc5ccccc5c4c3)c3oc4ccccc4c23)cc1]

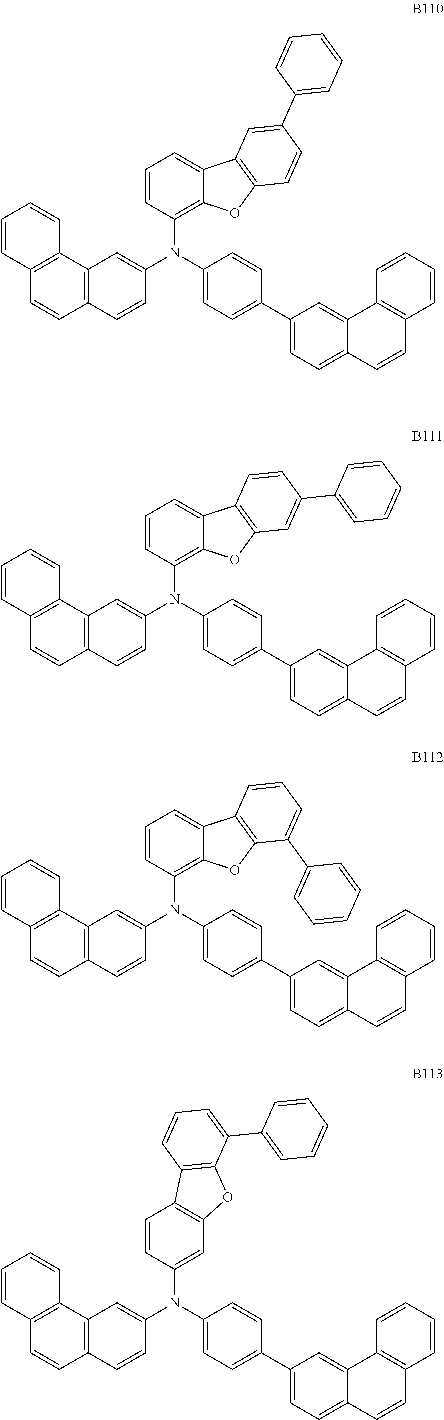 [SMILES: c1ccc(-c2ccc3oc4c(N(c5ccc(-c6ccc7ccc8ccccc8c7c6)cc5)c5ccc6ccc7ccccc7c6c5)cccc4c3c2)cc1 ; c1ccc(-c2ccc3c(c2)oc2c(N(c4ccc(-c5ccc6ccc7ccccc7c6c5)cc4)c4ccc5ccc6ccccc6c5c4)cccc23)cc1 ; c1ccc(-c2cccc3c2oc2c(N(c4ccc(-c5ccc6ccc7ccccc7c6c5)cc4)c4ccc5ccc6ccccc6c5c4)cccc23)cc1 ; c1ccc(-c2cccc3c2oc2cc(N(c4ccc(-c5ccc6ccc7ccccc7c6c5)cc4)c4ccc5ccc6ccccc6c5c4)ccc23)cc1]

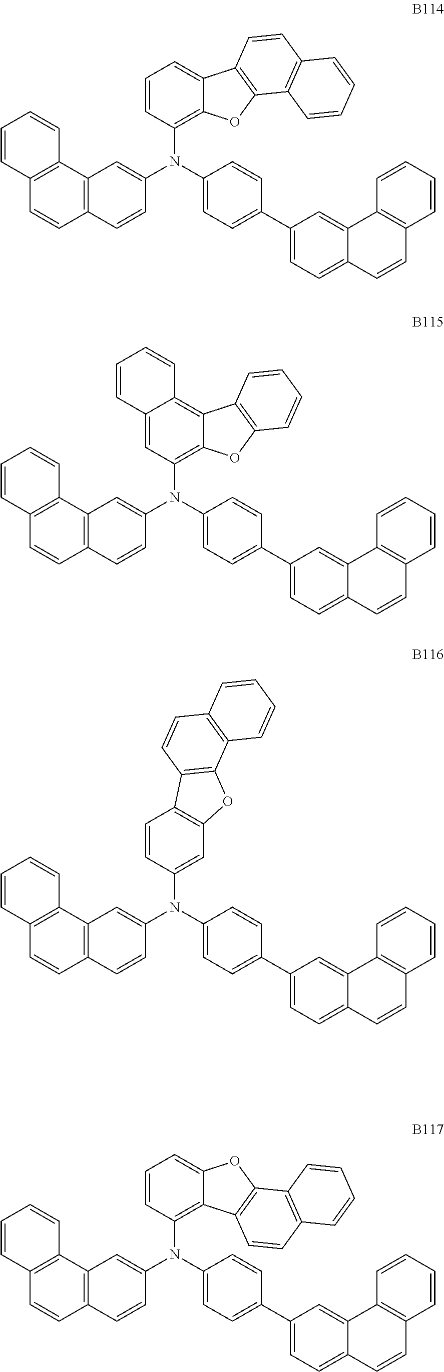 [SMILES: c1ccc2c(c1)ccc1ccc(-c3ccc(N(c4ccc5ccc6ccccc6c5c4)c4cccc5c4oc4c6ccccc6ccc54)cc3)cc12 ; c1ccc2c(c1)ccc1ccc(-c3ccc(N(c4ccc5ccc6ccccc6c5c4)c4cc5ccccc5c5c4oc4ccccc45)cc3)cc12 ; c1ccc2c(c1)ccc1ccc(-c3ccc(N(c4ccc5c(c4)oc4c6ccccc6ccc54)c4ccc5ccc6ccccc6c5c4)cc3)cc12 ; c1ccc2c(c1)ccc1ccc(-c3ccc(N(c4ccc5ccc6ccccc6c5c4)c4cccc5oc6c7ccccc7ccc6c45)cc3)cc12]

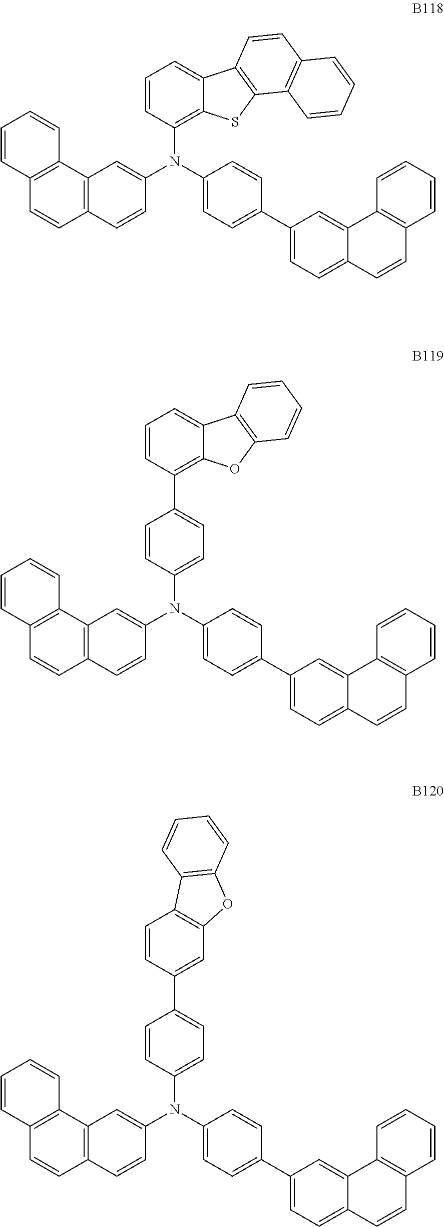 [SMILES: c1ccc2c(c1)ccc1ccc(-c3ccc(N(c4ccc5ccc6ccccc6c5c4)c4cccc5c4sc4c6ccccc6ccc54)cc3)cc12 ; c1ccc2c(c1)ccc1ccc(-c3ccc(N(c4ccc(-c5cccc6c5oc5ccccc56)cc4)c4ccc5ccc6ccccc6c5c4)cc3)cc12 ; c1ccc2c(c1)ccc1ccc(-c3ccc(N(c4ccc(-c5ccc6c(c5)oc5ccccc56)cc4)c4ccc5ccc6ccccc6c5c4)cc3)cc12]

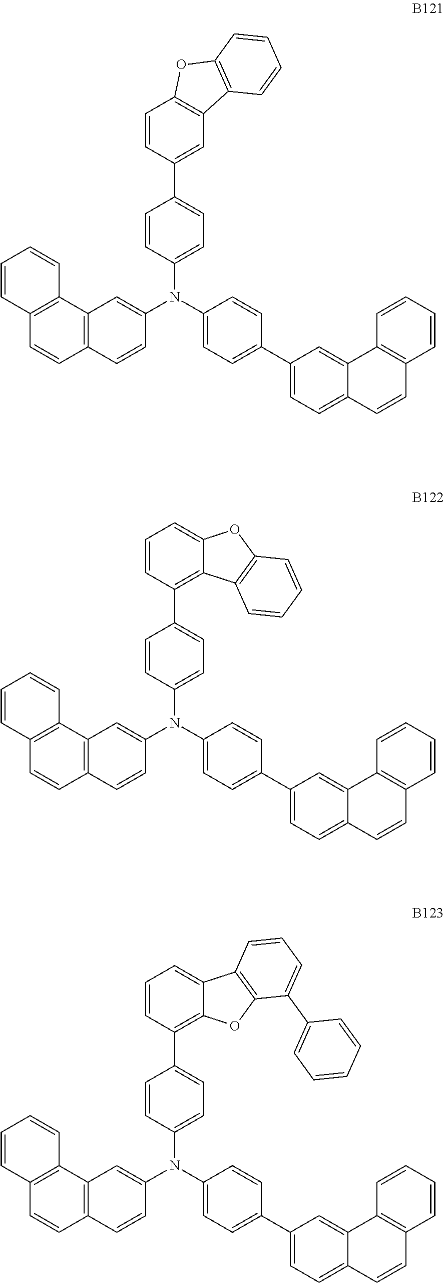 [SMILES: c1ccc2c(c1)ccc1ccc(-c3ccc(N(c4ccc(-c5ccc6oc7ccccc7c6c5)cc4)c4ccc5ccc6ccccc6c5c4)cc3)cc12 ; c1ccc2c(c1)ccc1ccc(-c3ccc(N(c4ccc(-c5cccc6oc7ccccc7c56)cc4)c4ccc5ccc6ccccc6c5c4)cc3)cc12 ; c1ccc(-c2cccc3c2oc2c(-c4ccc(N(c5ccc(-c6ccc7ccc8ccccc8c7c6)cc5)c5ccc6ccc7ccccc7c6c5)cc4)cccc23)cc1]

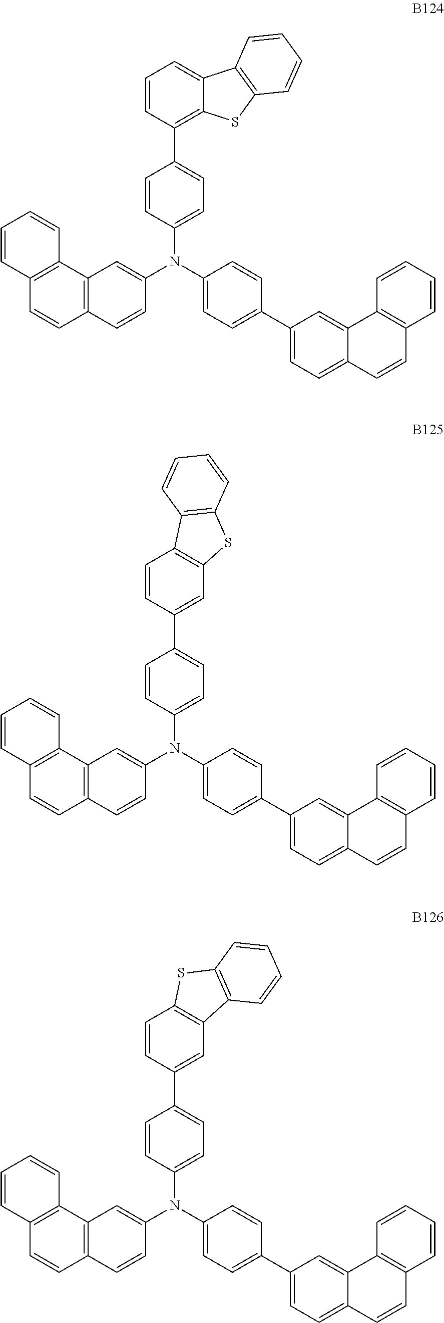 [SMILES: c1ccc2c(c1)ccc1ccc(-c3ccc(N(c4ccc(-c5cccc6c5sc5ccccc56)cc4)c4ccc5ccc6ccccc6c5c4)cc3)cc12 ; c1ccc2c(c1)ccc1ccc(-c3ccc(N(c4ccc(-c5ccc6c(c5)sc5ccccc56)cc4)c4ccc5ccc6ccccc6c5c4)cc3)cc12 ; c1ccc2c(c1)ccc1ccc(-c3ccc(N(c4ccc(-c5ccc6sc7ccccc7c6c5)cc4)c4ccc5ccc6ccccc6c5c4)cc3)cc12]

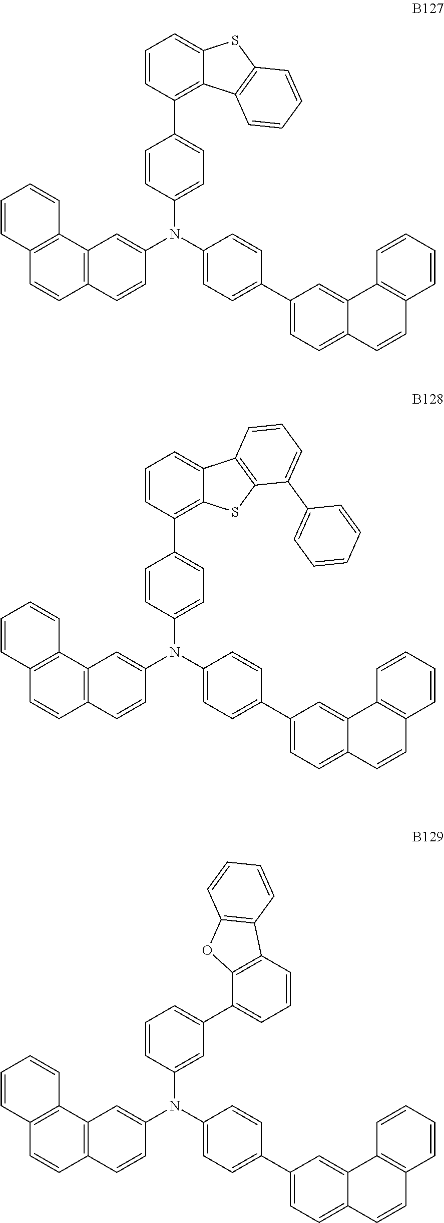 [SMILES: c1ccc2c(c1)ccc1ccc(-c3ccc(N(c4ccc(-c5cccc6sc7ccccc7c56)cc4)c4ccc5ccc6ccccc6c5c4)cc3)cc12 ; c1ccc(-c2cccc3c2sc2c(-c4ccc(N(c5ccc(-c6ccc7ccc8ccccc8c7c6)cc5)c5ccc6ccc7ccccc7c6c5)cc4)cccc23)cc1 ; c1cc(-c2cccc3c2oc2ccccc23)cc(N(c2ccc(-c3ccc4ccc5ccccc5c4c3)cc2)c2ccc3ccc4ccccc4c3c2)c1]

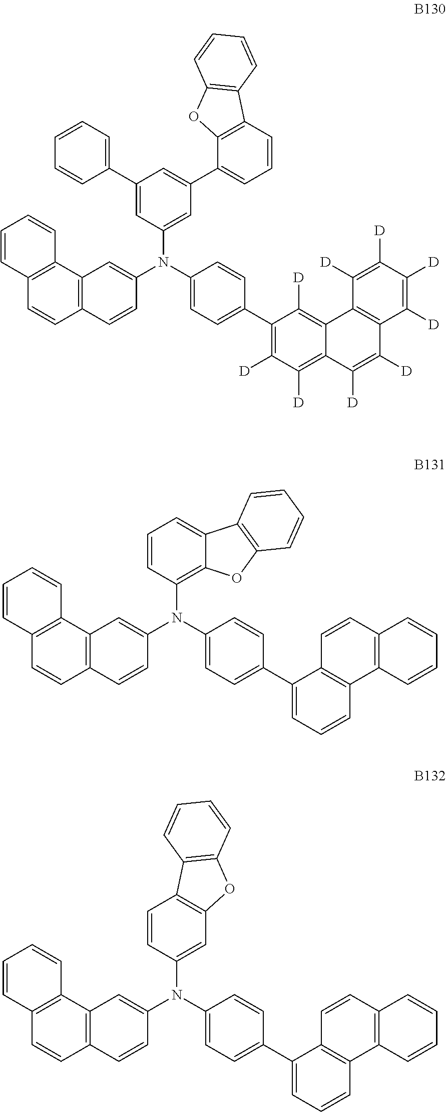 [SMILES: [2H]c1c([2H])c([2H])c2c(c1[2H])c([2H])c([2H])c1c([2H])c([2H])c(-c3ccc(N(c4cc(-c5ccccc5)cc(-c5cccc6c5oc5ccccc56)c4)c4ccc5ccc6ccccc6c5c4)cc3)c([2H])c12 ; c1ccc2c(c1)ccc1ccc(N(c3ccc(-c4cccc5c4ccc4ccccc45)cc3)c3cccc4c3oc3ccccc34)cc12 ; c1ccc2c(c1)ccc1ccc(N(c3ccc(-c4cccc5c4ccc4ccccc45)cc3)c3ccc4c(c3)oc3ccccc34)cc12]

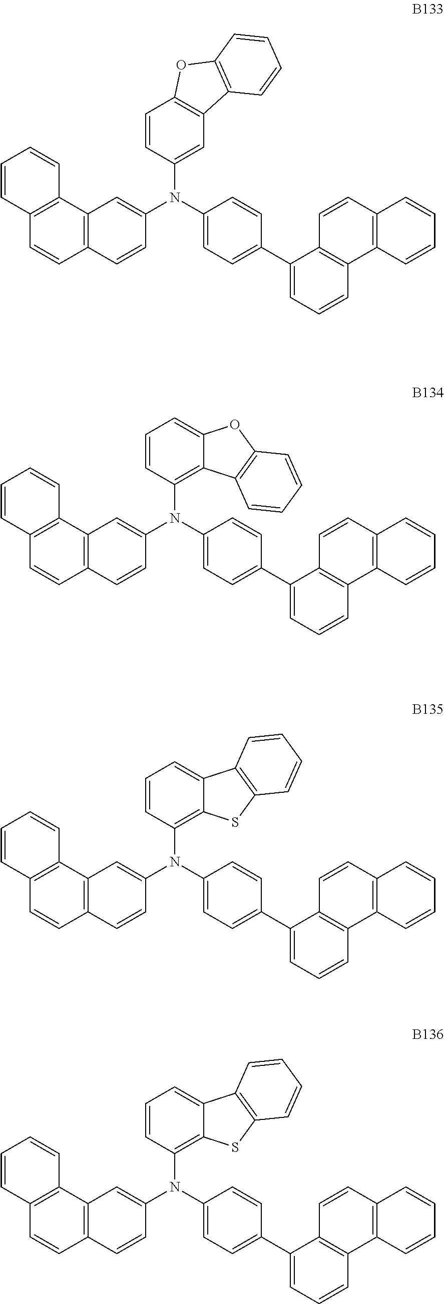 [SMILES: c1ccc2c(c1)ccc1ccc(N(c3ccc(-c4cccc5c4ccc4ccccc45)cc3)c3ccc4oc5ccccc5c4c3)cc12 ; c1ccc2c(c1)ccc1ccc(N(c3ccc(-c4cccc5c4ccc4ccccc45)cc3)c3cccc4oc5ccccc5c34)cc12 ; c1ccc2c(c1)ccc1ccc(N(c3ccc(-c4cccc5c4ccc4ccccc45)cc3)c3cccc4c3sc3ccccc34)cc12 ; c1ccc2c(c1)ccc1ccc(N(c3ccc(-c4cccc5c4ccc4ccccc45)cc3)c3cccc4c3sc3ccccc34)cc12]

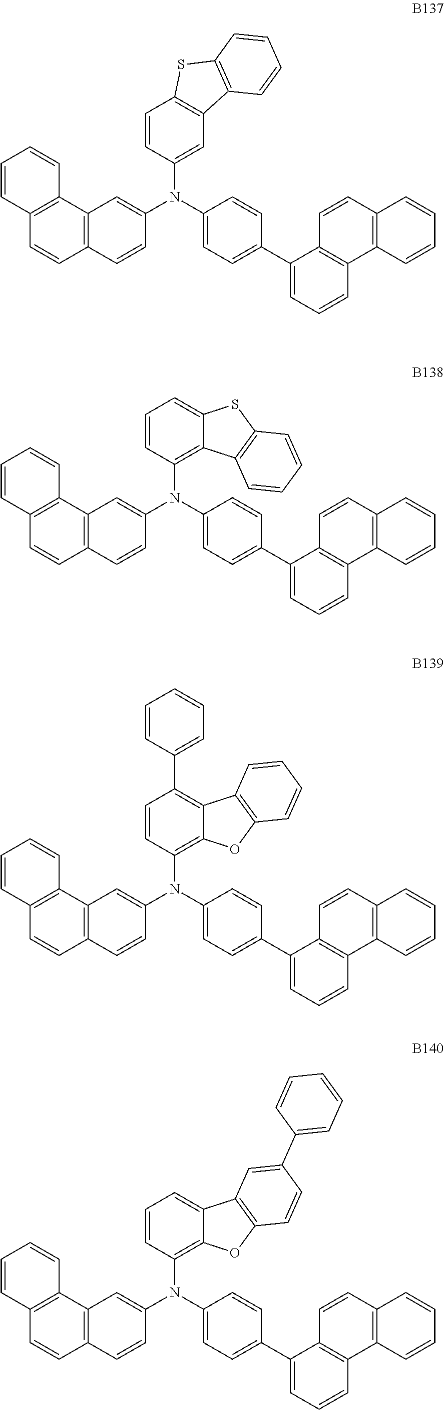 [SMILES: c1ccc2c(c1)ccc1ccc(N(c3ccc(-c4cccc5c4ccc4ccccc45)cc3)c3ccc4sc5ccccc5c4c3)cc12 ; c1ccc2c(c1)ccc1ccc(N(c3ccc(-c4cccc5c4ccc4ccccc45)cc3)c3cccc4sc5ccccc5c34)cc12 ; c1ccc(-c2ccc(N(c3ccc(-c4cccc5c4ccc4ccccc45)cc3)c3ccc4ccc5ccccc5c4c3)c3oc4ccccc4c23)cc1 ; c1ccc(-c2ccc3oc4c(N(c5ccc(-c6cccc7c6ccc6ccccc67)cc5)c5ccc6ccc7ccccc7c6c5)cccc4c3c2)cc1]

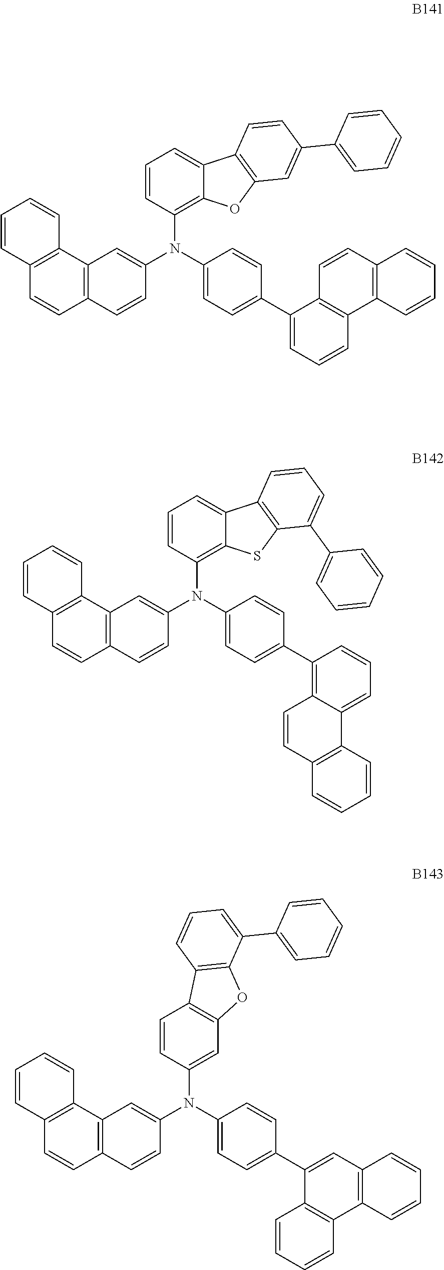

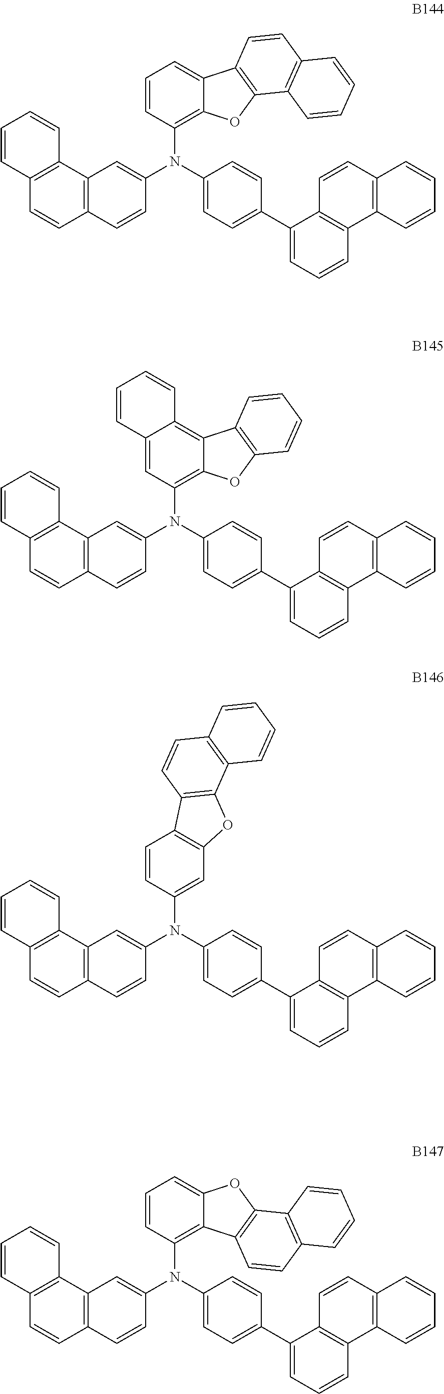 [SMILES: c1ccc2c(c1)ccc1ccc(N(c3ccc(-c4cccc5c4ccc4ccccc45)cc3)c3cccc4c3oc3c5ccccc5ccc43)cc12 ; c1ccc2c(c1)ccc1ccc(N(c3ccc(-c4cccc5c4ccc4ccccc45)cc3)c3cc4ccccc4c4c3oc3ccccc34)cc12 ; c1ccc2c(c1)ccc1ccc(N(c3ccc(-c4cccc5c4ccc4ccccc45)cc3)c3ccc4c(c3)oc3c5ccccc5ccc43)cc12 ; c1ccc2c(c1)ccc1ccc(N(c3ccc(-c4cccc5c4ccc4ccccc45)cc3)c3cccc4oc5c6ccccc6ccc5c34)cc12]

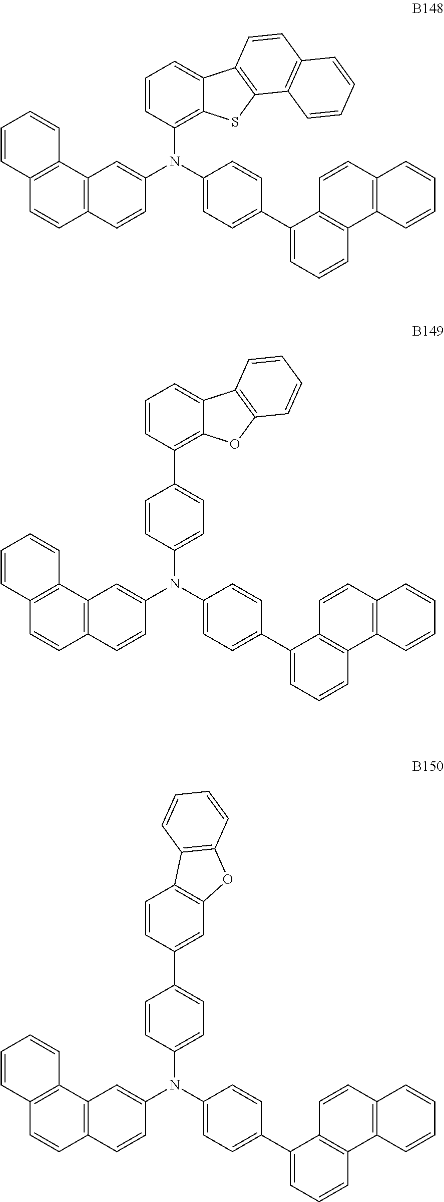 [SMILES: c1ccc2c(c1)ccc1ccc(N(c3ccc(-c4cccc5c4ccc4ccccc45)cc3)c3cccc4c3sc3c5ccccc5ccc43)cc12 ; c1ccc2c(c1)ccc1ccc(N(c3ccc(-c4cccc5c4ccc4ccccc45)cc3)c3ccc(-c4cccc5c4oc4ccccc45)cc3)cc12 ; c1ccc2c(c1)ccc1ccc(N(c3ccc(-c4ccc5c(c4)oc4ccccc45)cc3)c3ccc(-c4cccc5c4ccc4ccccc45)cc3)cc12]

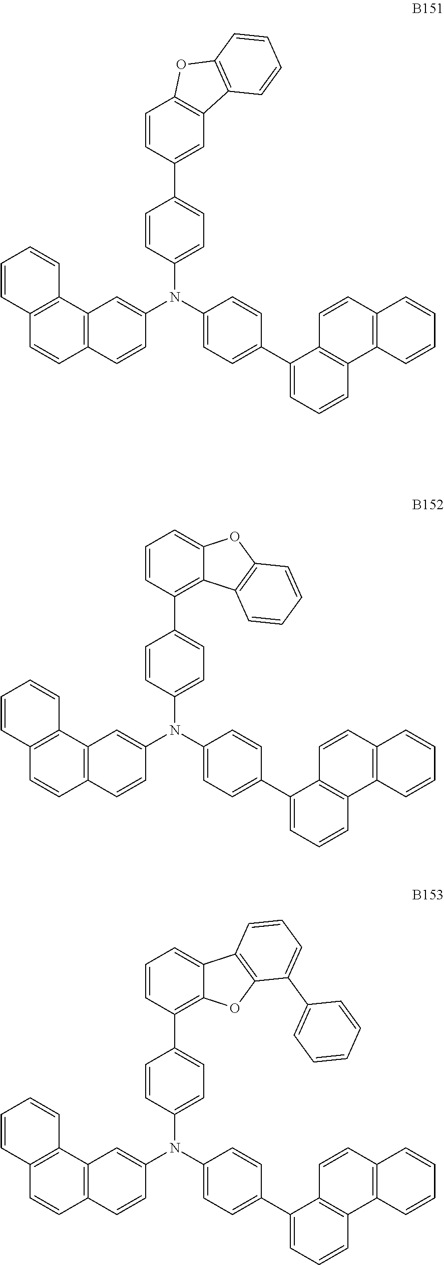 [SMILES: c1ccc2c(c1)ccc1ccc(N(c3ccc(-c4ccc5oc6ccccc6c5c4)cc3)c3ccc(-c4cccc5c4ccc4ccccc45)cc3)cc12 ; c1ccc2c(c1)ccc1ccc(N(c3ccc(-c4cccc5c4ccc4ccccc45)cc3)c3ccc(-c4cccc5oc6ccccc6c45)cc3)cc12 ; c1ccc(-c2cccc3c2oc2c(-c4ccc(N(c5ccc(-c6cccc7c6ccc6ccccc67)cc5)c5ccc6ccc7ccccc7c6c5)cc4)cccc23)cc1]

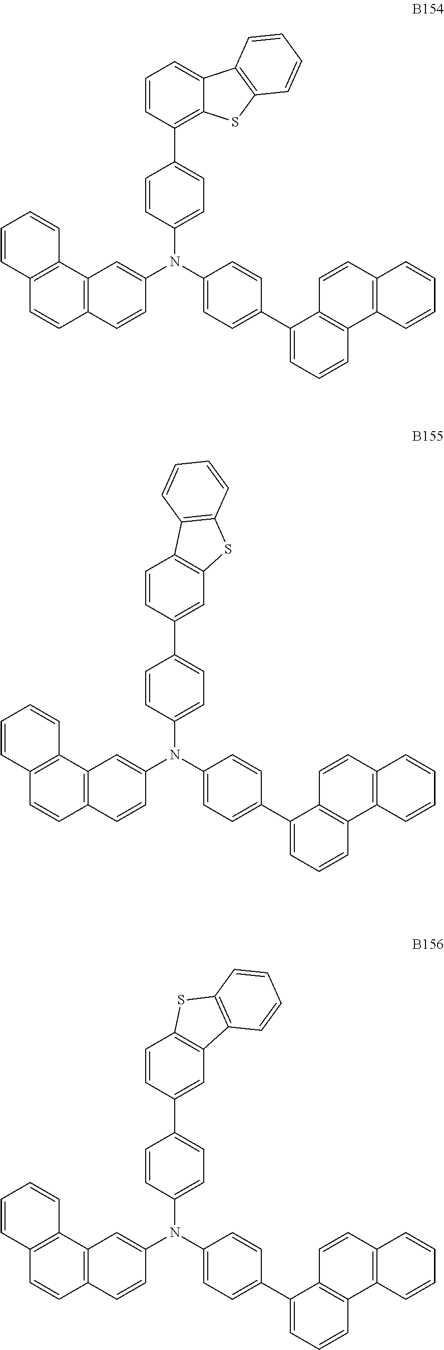 [SMILES: c1ccc2c(c1)ccc1ccc(N(c3ccc(-c4cccc5c4ccc4ccccc45)cc3)c3ccc(-c4cccc5c4sc4ccccc45)cc3)cc12 ; c1ccc2c(c1)ccc1ccc(N(c3ccc(-c4ccc5c(c4)sc4ccccc45)cc3)c3ccc(-c4cccc5c4ccc4ccccc45)cc3)cc12 ; c1ccc2c(c1)ccc1ccc(N(c3ccc(-c4ccc5sc6ccccc6c5c4)cc3)c3ccc(-c4cccc5c4ccc4ccccc45)cc3)cc12]

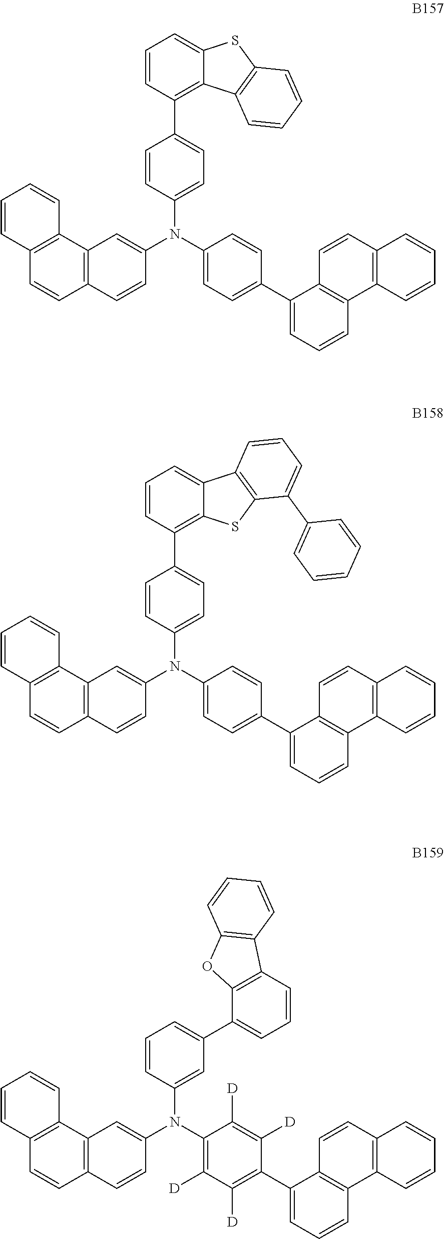 [SMILES: c1ccc2c(c1)ccc1ccc(N(c3ccc(-c4cccc5c4ccc4ccccc45)cc3)c3ccc(-c4cccc5sc6ccccc6c45)cc3)cc12 ; c1ccc(-c2cccc3c2sc2c(-c4ccc(N(c5ccc(-c6cccc7c6ccc6ccccc67)cc5)c5ccc6ccc7ccccc7c6c5)cc4)cccc23)cc1 ; [2H]c1c([2H])c(N(c2cccc(-c3cccc4c3oc3ccccc34)c2)c2ccc3ccc4ccccc4c3c2)c([2H])c([2H])c1-c1cccc2c1ccc1ccccc12]

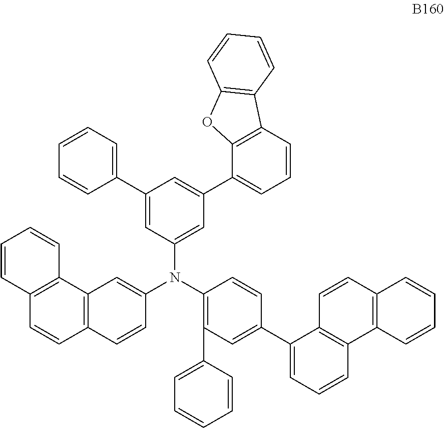 [SMILES: c1ccc(-c2cc(-c3cccc4c3oc3ccccc34)cc(N(c3ccc4ccc5ccccc5c4c3)c3ccc(-c4cccc5c4ccc4ccccc45)cc3-c3ccccc3)c2)cc1]

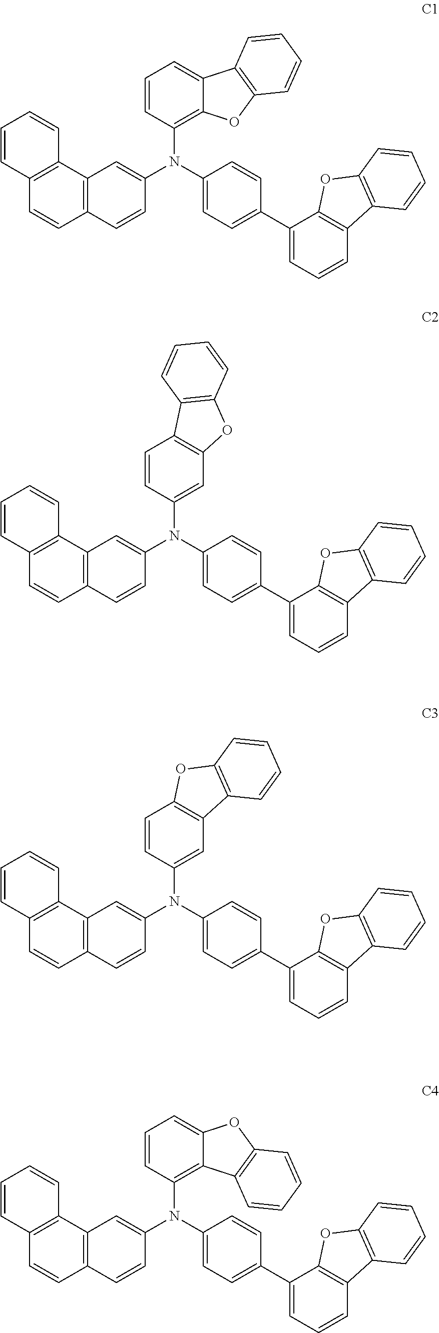 [SMILES: c1ccc2c(c1)ccc1ccc(N(c3ccc(-c4cccc5c4oc4ccccc45)cc3)c3cccc4c3oc3ccccc34)cc12 ; c1ccc2c(c1)ccc1ccc(N(c3ccc(-c4cccc5c4oc4ccccc45)cc3)c3ccc4c(c3)oc3ccccc34)cc12 ; c1ccc2c(c1)ccc1ccc(N(c3ccc(-c4cccc5c4oc4ccccc45)cc3)c3ccc4oc5ccccc5c4c3)cc12 ; c1ccc2c(c1)ccc1ccc(N(c3ccc(-c4cccc5c4oc4ccccc45)cc3)c3cccc4oc5ccccc5c34)cc12]

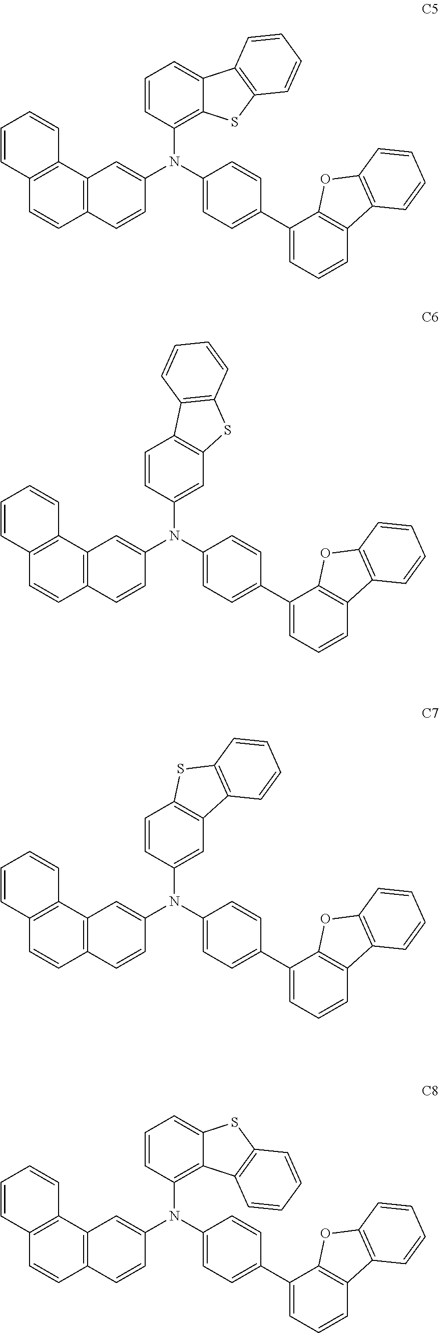 [SMILES: c1ccc2c(c1)ccc1ccc(N(c3ccc(-c4cccc5c4oc4ccccc45)cc3)c3cccc4c3sc3ccccc34)cc12 ; c1ccc2c(c1)ccc1ccc(N(c3ccc(-c4cccc5c4oc4ccccc45)cc3)c3ccc4c(c3)sc3ccccc34)cc12 ; c1ccc2c(c1)ccc1ccc(N(c3ccc(-c4cccc5c4oc4ccccc45)cc3)c3ccc4sc5ccccc5c4c3)cc12 ; c1ccc2c(c1)ccc1ccc(N(c3ccc(-c4cccc5c4oc4ccccc45)cc3)c3cccc4sc5ccccc5c34)cc12]

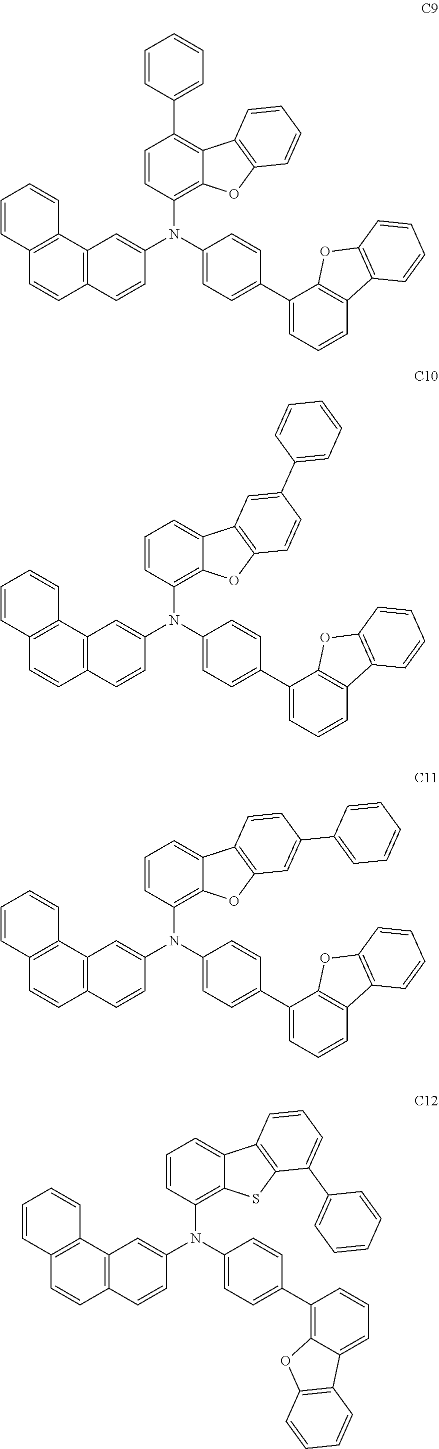 [SMILES: c1ccc(-c2ccc(N(c3ccc(-c4cccc5c4oc4ccccc45)cc3)c3ccc4ccc5ccccc5c4c3)c3oc4ccccc4c23)cc1 ; c1ccc(-c2ccc3oc4c(N(c5ccc(-c6cccc7c6oc6ccccc67)cc5)c5ccc6ccc7ccccc7c6c5)cccc4c3c2)cc1 ; c1ccc(-c2ccc3c(c2)oc2c(N(c4ccc(-c5cccc6c5oc5ccccc56)cc4)c4ccc5ccc6ccccc6c5c4)cccc23)cc1 ; c1ccc(-c2cccc3c2sc2c(N(c4ccc(-c5cccc6c5oc5ccccc56)cc4)c4ccc5ccc6ccccc6c5c4)cccc23)cc1]

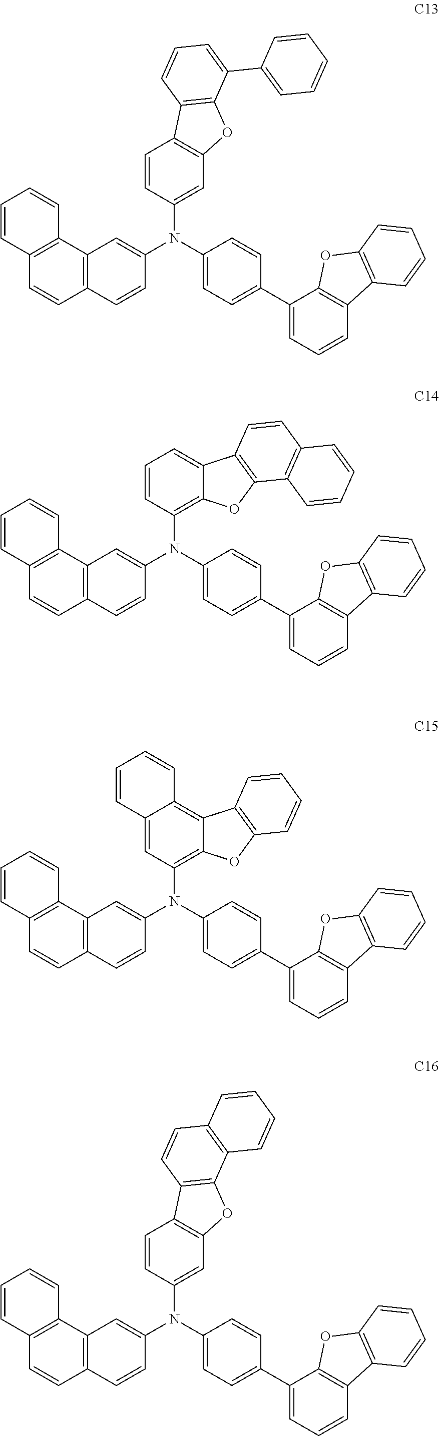 [SMILES: c1ccc(-c2cccc3c2oc2cc(N(c4ccc(-c5cccc6c5oc5ccccc56)cc4)c4ccc5ccc6ccccc6c5c4)ccc23)cc1 ; c1ccc2c(c1)ccc1ccc(N(c3ccc(-c4cccc5c4oc4ccccc45)cc3)c3cccc4c3oc3c5ccccc5ccc43)cc12 ; c1ccc2c(c1)ccc1ccc(N(c3ccc(-c4cccc5c4oc4ccccc45)cc3)c3cc4ccccc4c4c3oc3ccccc34)cc12 ; c1ccc2c(c1)ccc1ccc(N(c3ccc(-c4cccc5c4oc4ccccc45)cc3)c3ccc4c(c3)oc3c5ccccc5ccc43)cc12]

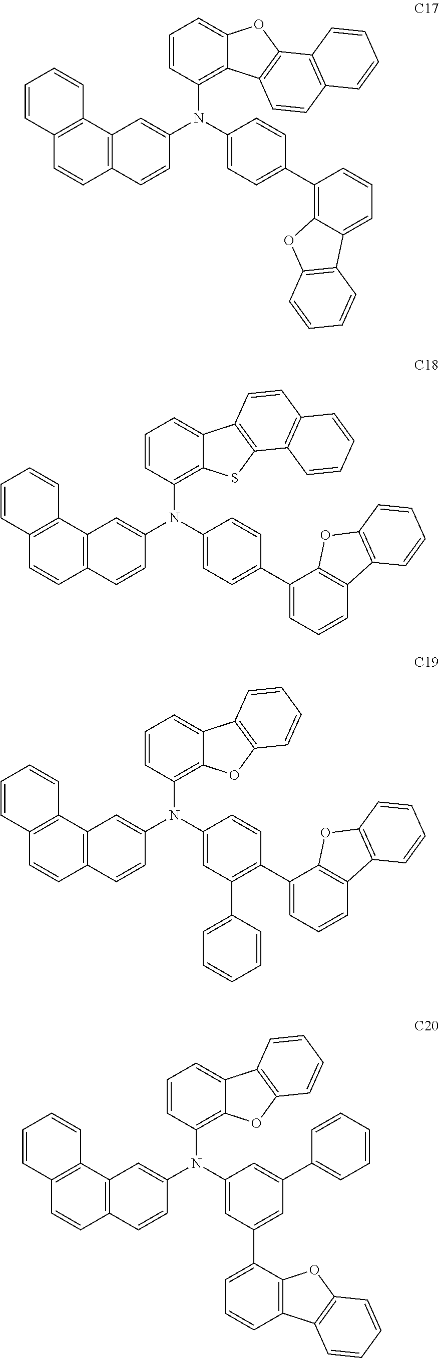 [SMILES: c1ccc2c(c1)ccc1ccc(N(c3ccc(-c4cccc5c4oc4ccccc45)cc3)c3cccc4oc5c6ccccc6ccc5c34)cc12 ; c1ccc2c(c1)ccc1ccc(N(c3ccc(-c4cccc5c4oc4ccccc45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2cccc3c2oc2ccccc23)cc1 ; c1ccc(-c2cc(-c3cccc4c3oc3ccccc34)cc(N(c3ccc4ccc5ccccc5c4c3)c3cccc4c3oc3ccccc34)c2)cc1]

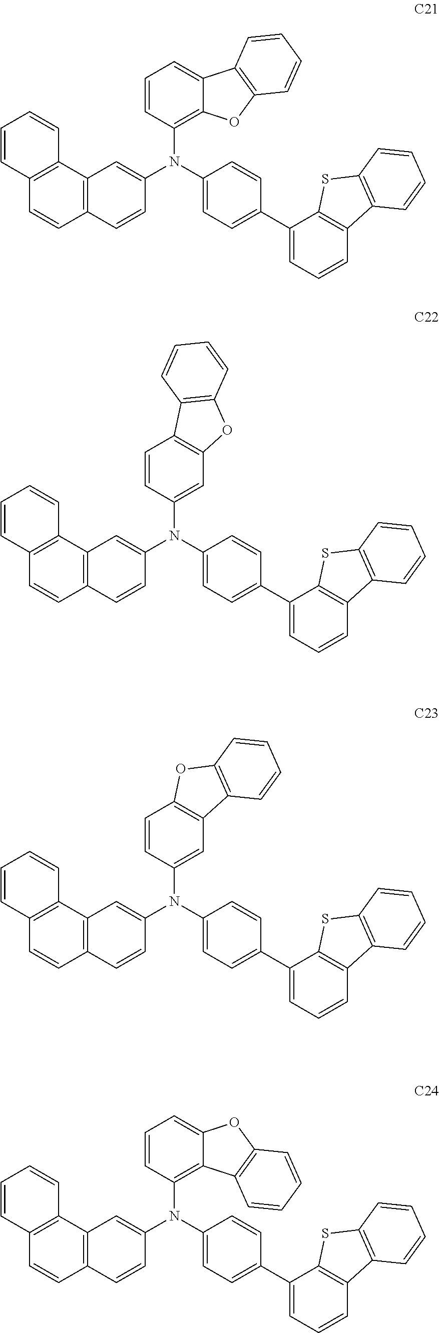 [SMILES: c1ccc2c(c1)ccc1ccc(N(c3ccc(-c4cccc5c4sc4ccccc45)cc3)c3cccc4c3oc3ccccc34)cc12 ; c1ccc2c(c1)ccc1ccc(N(c3ccc(-c4cccc5c4sc4ccccc45)cc3)c3ccc4c(c3)oc3ccccc34)cc12 ; c1ccc2c(c1)ccc1ccc(N(c3ccc(-c4cccc5c4sc4ccccc45)cc3)c3ccc4oc5ccccc5c4c3)cc12 ; c1ccc2c(c1)ccc1ccc(N(c3ccc(-c4cccc5c4sc4ccccc45)cc3)c3cccc4oc5ccccc5c34)cc12]

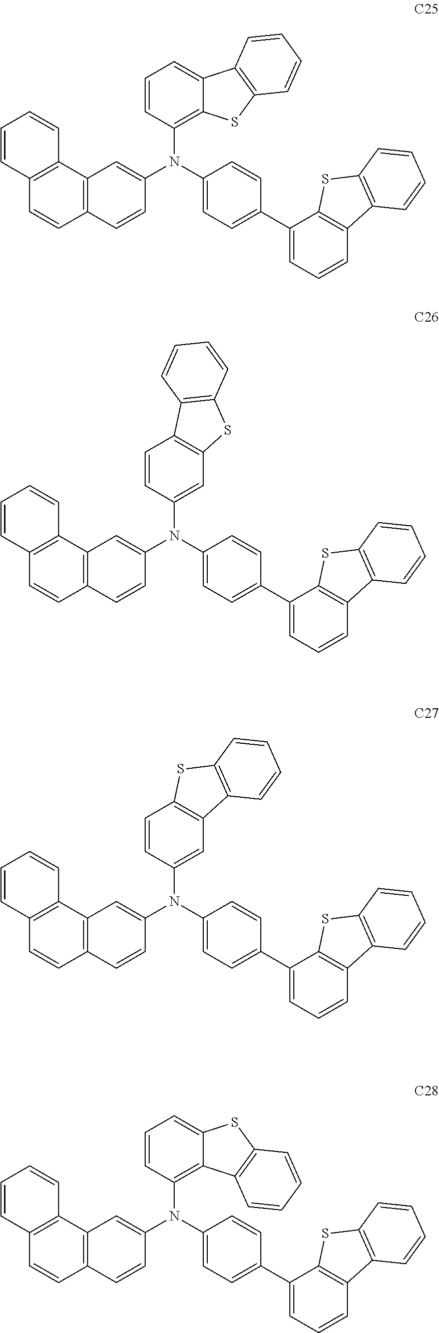 [SMILES: c1ccc2c(c1)ccc1ccc(N(c3ccc(-c4cccc5c4sc4ccccc45)cc3)c3cccc4c3sc3ccccc34)cc12 ; c1ccc2c(c1)ccc1ccc(N(c3ccc(-c4cccc5c4sc4ccccc45)cc3)c3ccc4c(c3)sc3ccccc34)cc12 ; c1ccc2c(c1)ccc1ccc(N(c3ccc(-c4cccc5c4sc4ccccc45)cc3)c3ccc4sc5ccccc5c4c3)cc12 ; c1ccc2c(c1)ccc1ccc(N(c3ccc(-c4cccc5c4sc4ccccc45)cc3)c3cccc4sc5ccccc5c34)cc12]

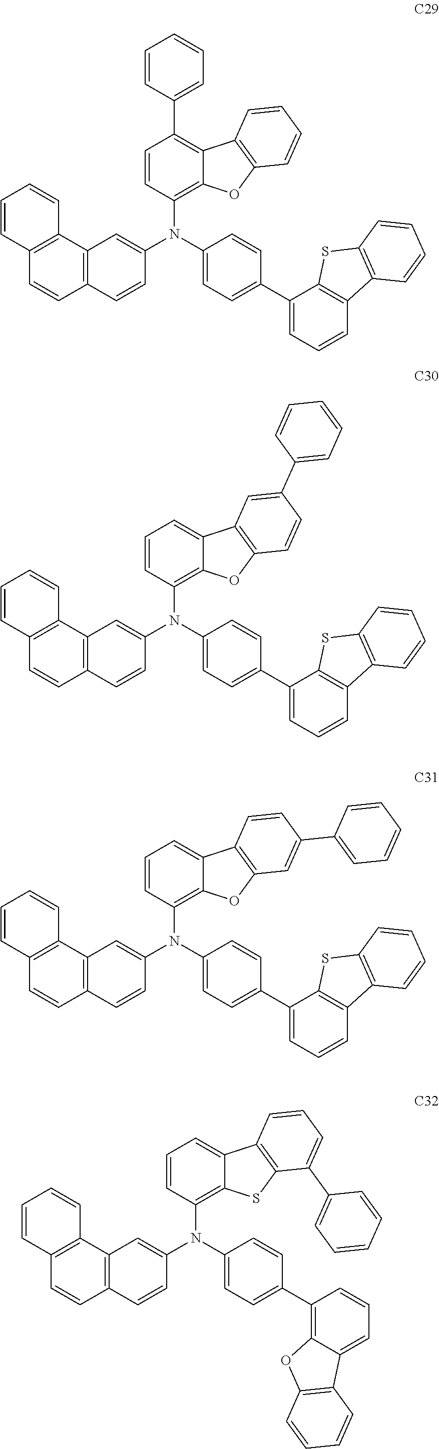 [SMILES: c1ccc(-c2ccc(N(c3ccc(-c4cccc5c4sc4ccccc45)cc3)c3ccc4ccc5ccccc5c4c3)c3oc4ccccc4c23)cc1 ; c1ccc(-c2ccc3oc4c(N(c5ccc(-c6cccc7c6sc6ccccc67)cc5)c5ccc6ccc7ccccc7c6c5)cccc4c3c2)cc1 ; c1ccc(-c2ccc3c(c2)oc2c(N(c4ccc(-c5cccc6c5sc5ccccc56)cc4)c4ccc5ccc6ccccc6c5c4)cccc23)cc1 ; c1ccc(-c2cccc3c2sc2c(N(c4ccc(-c5cccc6c5oc5ccccc56)cc4)c4ccc5ccc6ccccc6c5c4)cccc23)cc1]

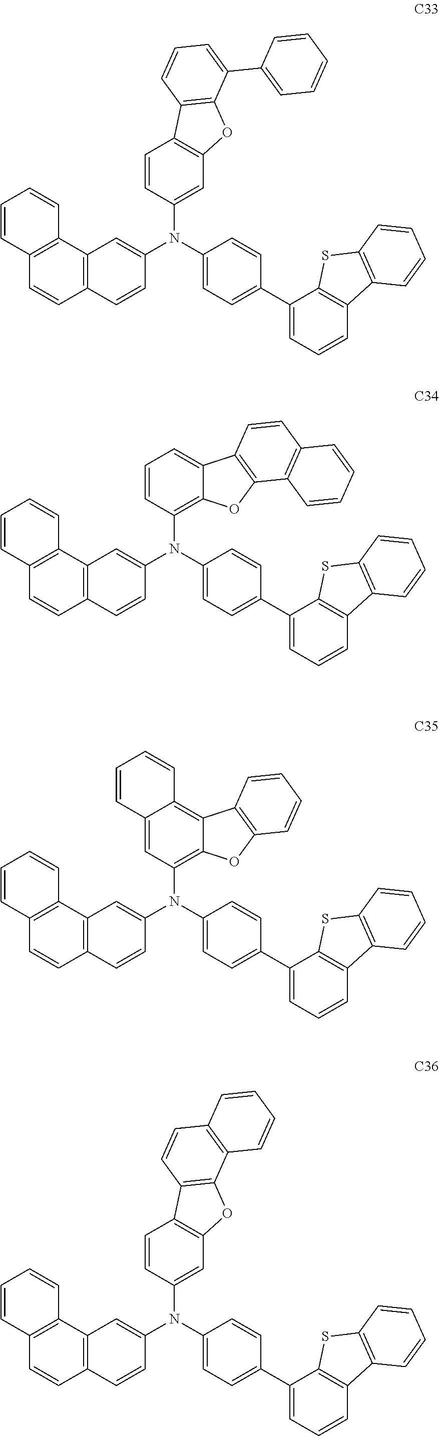 [SMILES: c1ccc(-c2cccc3c2oc2cc(N(c4ccc(-c5cccc6c5sc5ccccc56)cc4)c4ccc5ccc6ccccc6c5c4)ccc23)cc1 ; c1ccc2c(c1)ccc1ccc(N(c3ccc(-c4cccc5c4sc4ccccc45)cc3)c3cccc4c3oc3c5ccccc5ccc43)cc12 ; c1ccc2c(c1)ccc1ccc(N(c3ccc(-c4cccc5c4sc4ccccc45)cc3)c3cc4ccccc4c4c3oc3ccccc34)cc12 ; c1ccc2c(c1)ccc1ccc(N(c3ccc(-c4cccc5c4sc4ccccc45)cc3)c3ccc4c(c3)oc3c5ccccc5ccc43)cc12]

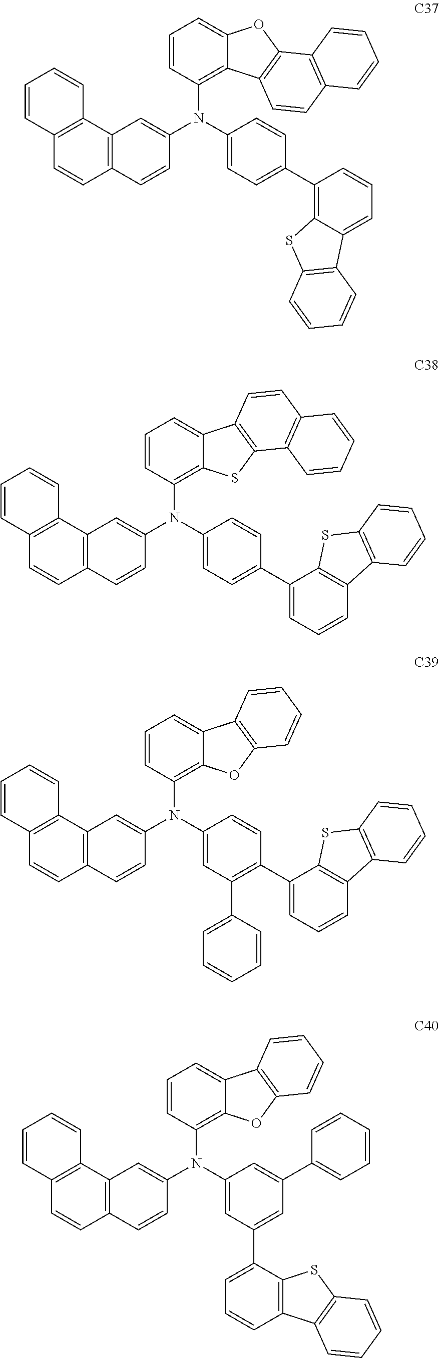 [SMILES: c1ccc2c(c1)ccc1ccc(N(c3ccc(-c4cccc5c4sc4ccccc45)cc3)c3cccc4oc5c6ccccc6ccc5c34)cc12 ; c1ccc2c(c1)ccc1ccc(N(c3ccc(-c4cccc5c4sc4ccccc45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2cccc3c2sc2ccccc23)cc1 ; c1ccc(-c2cc(-c3cccc4c3sc3ccccc34)cc(N(c3ccc4ccc5ccccc5c4c3)c3cccc4c3oc3ccccc34)c2)cc1]

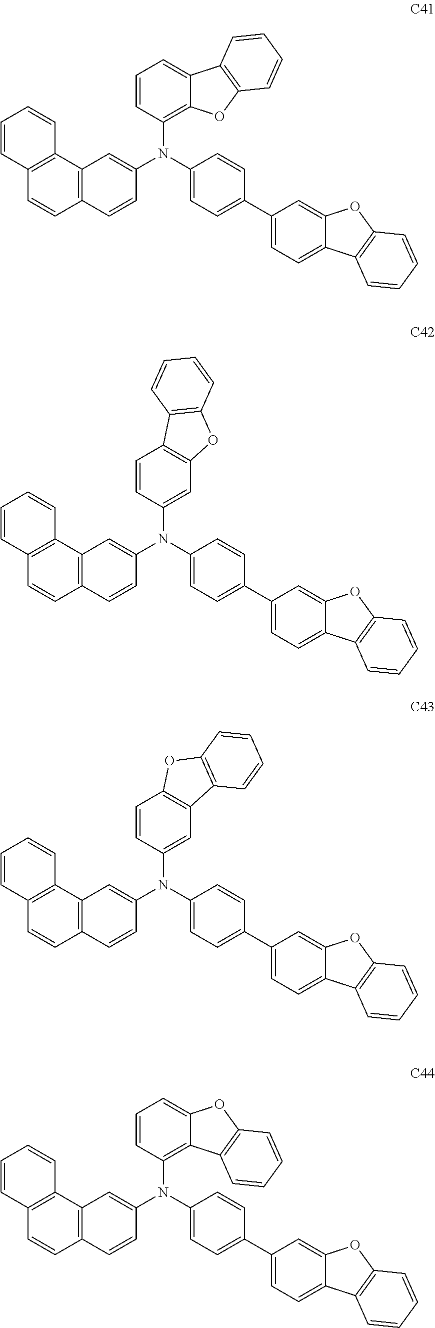 [SMILES: c1ccc2c(c1)ccc1ccc(N(c3ccc(-c4ccc5c(c4)oc4ccccc45)cc3)c3cccc4c3oc3ccccc34)cc12 ; c1ccc2c(c1)ccc1ccc(N(c3ccc(-c4ccc5c(c4)oc4ccccc45)cc3)c3ccc4c(c3)oc3ccccc34)cc12 ; c1ccc2c(c1)ccc1ccc(N(c3ccc(-c4ccc5c(c4)oc4ccccc45)cc3)c3ccc4oc5ccccc5c4c3)cc12 ; c1ccc2c(c1)ccc1ccc(N(c3ccc(-c4ccc5c(c4)oc4ccccc45)cc3)c3cccc4oc5ccccc5c34)cc12]

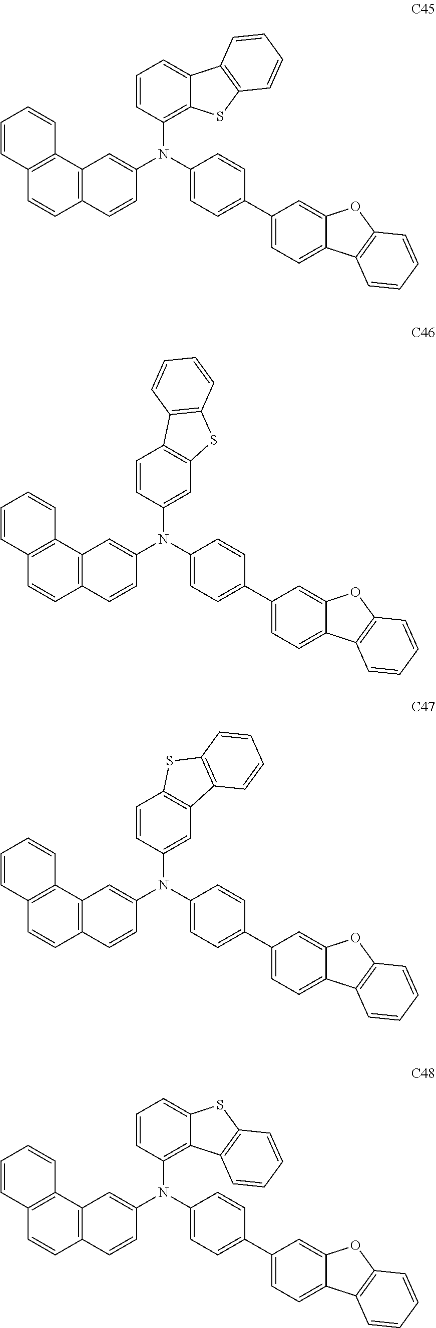 [SMILES: c1ccc2c(c1)ccc1ccc(N(c3ccc(-c4ccc5c(c4)oc4ccccc45)cc3)c3cccc4c3sc3ccccc34)cc12 ; c1ccc2c(c1)ccc1ccc(N(c3ccc(-c4ccc5c(c4)oc4ccccc45)cc3)c3ccc4c(c3)sc3ccccc34)cc12 ; c1ccc2c(c1)ccc1ccc(N(c3ccc(-c4ccc5c(c4)oc4ccccc45)cc3)c3ccc4sc5ccccc5c4c3)cc12 ; c1ccc2c(c1)ccc1ccc(N(c3ccc(-c4ccc5c(c4)oc4ccccc45)cc3)c3cccc4sc5ccccc5c34)cc12]

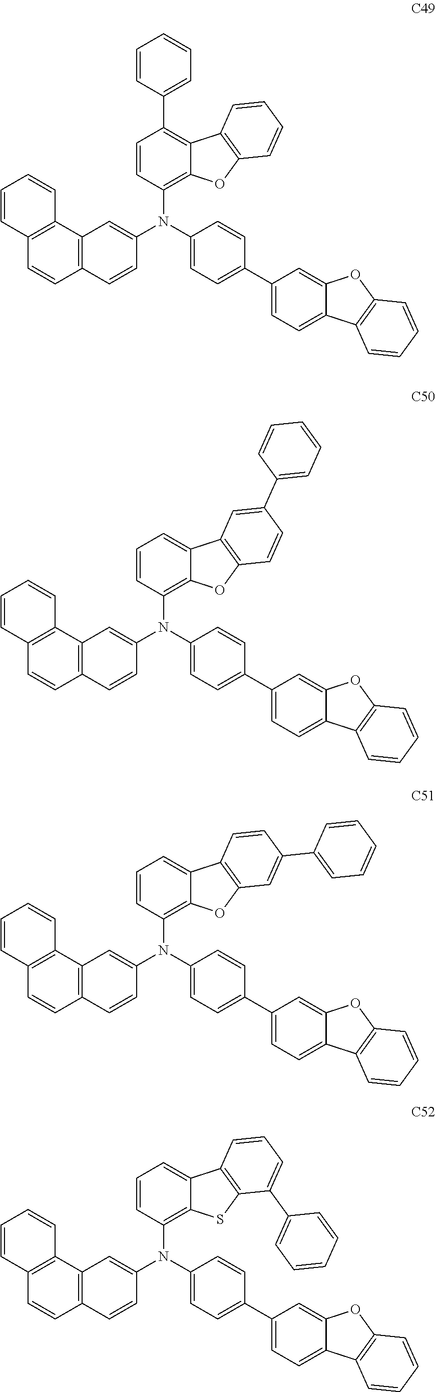 [SMILES: c1ccc(-c2ccc(N(c3ccc(-c4ccc5c(c4)oc4ccccc45)cc3)c3ccc4ccc5ccccc5c4c3)c3oc4ccccc4c23)cc1 ; c1ccc(-c2ccc3oc4c(N(c5ccc(-c6ccc7c(c6)oc6ccccc67)cc5)c5ccc6ccc7ccccc7c6c5)cccc4c3c2)cc1 ; c1ccc(-c2ccc3c(c2)oc2c(N(c4ccc(-c5ccc6c(c5)oc5ccccc56)cc4)c4ccc5ccc6ccccc6c5c4)cccc23)cc1 ; c1ccc(-c2cccc3c2sc2c(N(c4ccc(-c5ccc6c(c5)oc5ccccc56)cc4)c4ccc5ccc6ccccc6c5c4)cccc23)cc1]

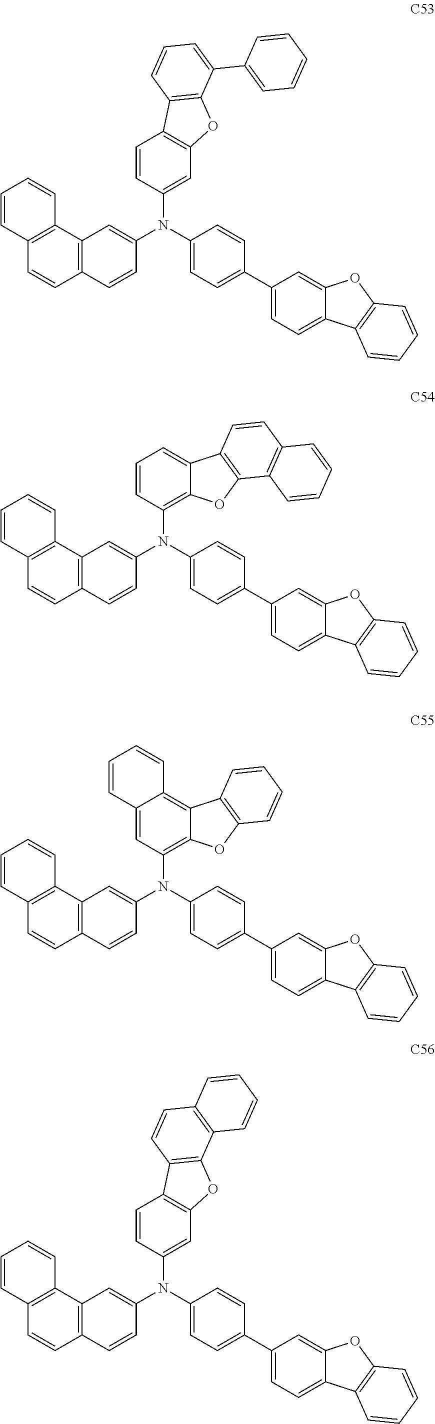 [SMILES: c1ccc(-c2cccc3c2oc2cc(N(c4ccc(-c5ccc6c(c5)oc5ccccc56)cc4)c4ccc5ccc6ccccc6c5c4)ccc23)cc1 ; c1ccc2c(c1)ccc1ccc(N(c3ccc(-c4ccc5c(c4)oc4ccccc45)cc3)c3cccc4c3oc3c5ccccc5ccc43)cc12 ; c1ccc2c(c1)ccc1ccc(N(c3ccc(-c4ccc5c(c4)oc4ccccc45)cc3)c3cc4ccccc4c4c3oc3ccccc34)cc12 ; c1ccc2c(c1)ccc1ccc(N(c3ccc(-c4ccc5c(c4)oc4ccccc45)cc3)c3ccc4c(c3)oc3c5ccccc5ccc43)cc12]

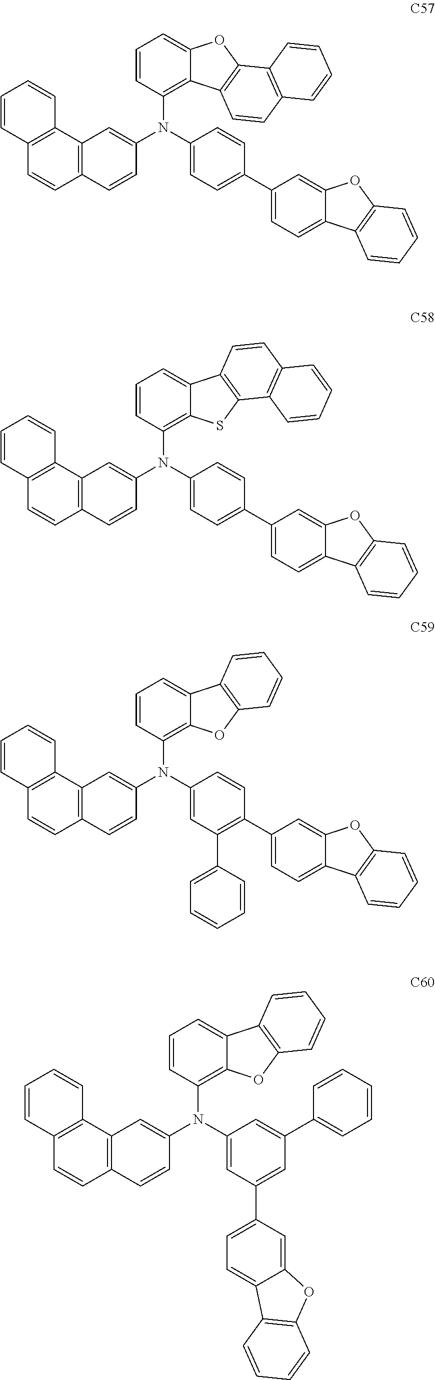 [SMILES: c1ccc2c(c1)ccc1ccc(N(c3ccc(-c4ccc5c(c4)oc4ccccc45)cc3)c3cccc4oc5c6ccccc6ccc5c34)cc12 ; c1ccc2c(c1)ccc1ccc(N(c3ccc(-c4ccc5c(c4)oc4ccccc45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2ccc3c(c2)oc2ccccc23)cc1 ; c1ccc(-c2cc(-c3ccc4c(c3)oc3ccccc34)cc(N(c3ccc4ccc5ccccc5c4c3)c3cccc4c3oc3ccccc34)c2)cc1]

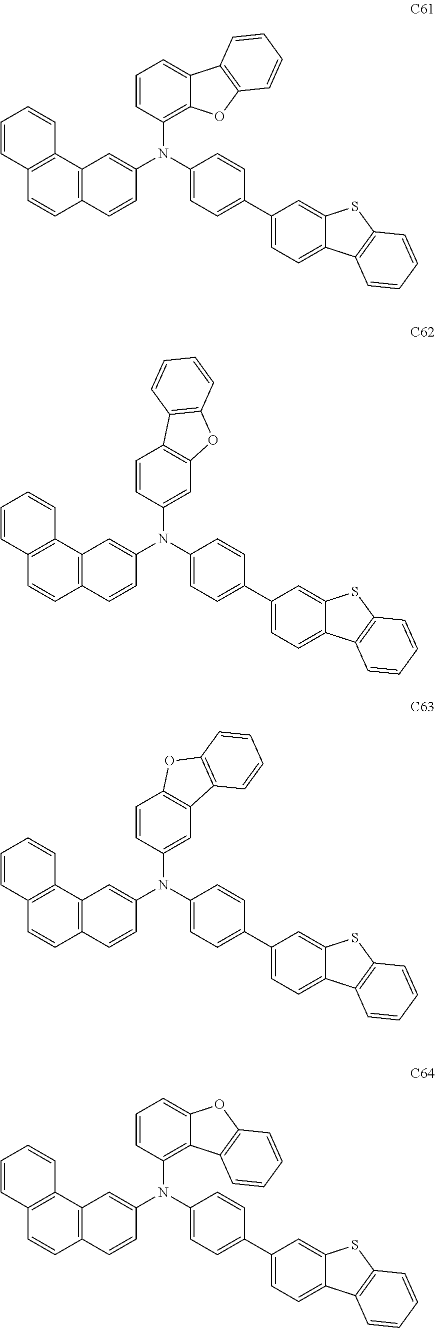 [SMILES: c1ccc2c(c1)ccc1ccc(N(c3ccc(-c4ccc5c(c4)sc4ccccc45)cc3)c3cccc4c3oc3ccccc34)cc12 ; c1ccc2c(c1)ccc1ccc(N(c3ccc(-c4ccc5c(c4)sc4ccccc45)cc3)c3ccc4c(c3)oc3ccccc34)cc12 ; c1ccc2c(c1)ccc1ccc(N(c3ccc(-c4ccc5c(c4)sc4ccccc45)cc3)c3ccc4oc5ccccc5c4c3)cc12 ; c1ccc2c(c1)ccc1ccc(N(c3ccc(-c4ccc5c(c4)sc4ccccc45)cc3)c3cccc4oc5ccccc5c34)cc12]

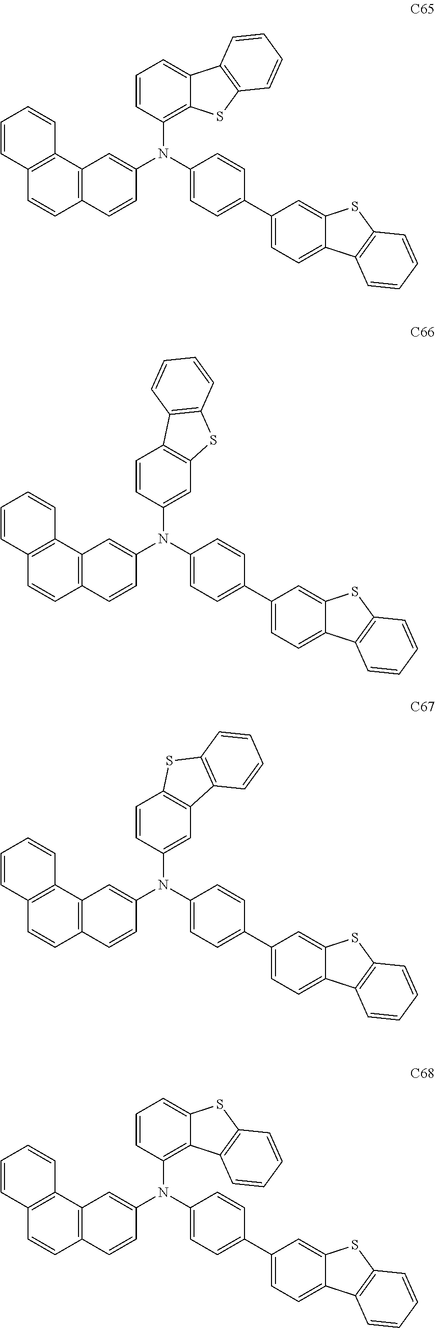 [SMILES: c1ccc2c(c1)ccc1ccc(N(c3ccc(-c4ccc5c(c4)sc4ccccc45)cc3)c3cccc4c3sc3ccccc34)cc12 ; c1ccc2c(c1)ccc1ccc(N(c3ccc(-c4ccc5c(c4)sc4ccccc45)cc3)c3ccc4c(c3)sc3ccccc34)cc12 ; c1ccc2c(c1)ccc1ccc(N(c3ccc(-c4ccc5c(c4)sc4ccccc45)cc3)c3ccc4sc5ccccc5c4c3)cc12 ; c1ccc2c(c1)ccc1ccc(N(c3ccc(-c4ccc5c(c4)sc4ccccc45)cc3)c3cccc4sc5ccccc5c34)cc12]

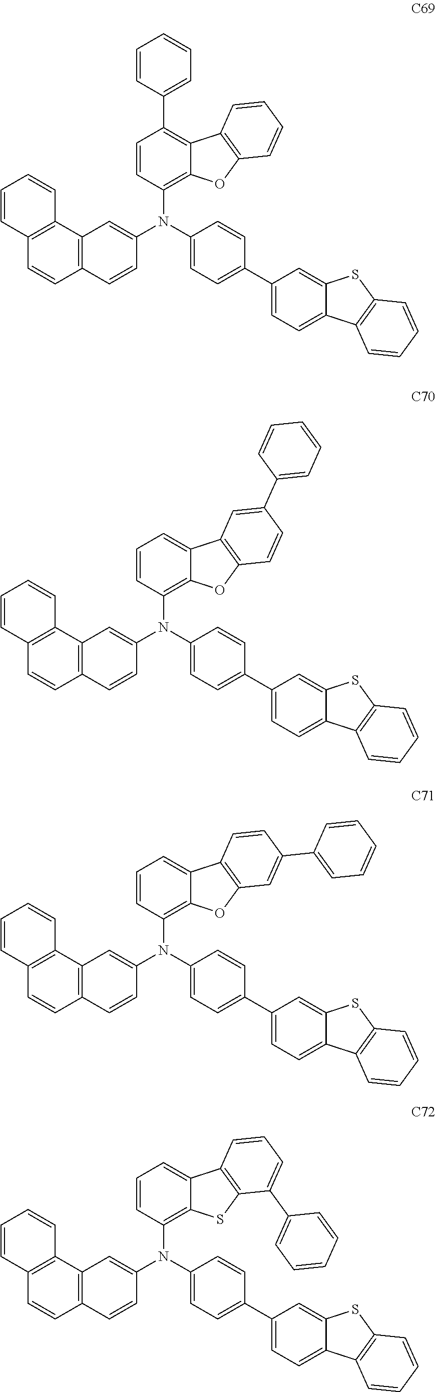 [SMILES: c1ccc(-c2ccc(N(c3ccc(-c4ccc5c(c4)sc4ccccc45)cc3)c3ccc4ccc5ccccc5c4c3)c3oc4ccccc4c23)cc1 ; c1ccc(-c2ccc3oc4c(N(c5ccc(-c6ccc7c(c6)sc6ccccc67)cc5)c5ccc6ccc7ccccc7c6c5)cccc4c3c2)cc1 ; c1ccc(-c2ccc3c(c2)oc2c(N(c4ccc(-c5ccc6c(c5)sc5ccccc56)cc4)c4ccc5ccc6ccccc6c5c4)cccc23)cc1 ; c1ccc(-c2cccc3c2sc2c(N(c4ccc(-c5ccc6c(c5)sc5ccccc56)cc4)c4ccc5ccc6ccccc6c5c4)cccc23)cc1]

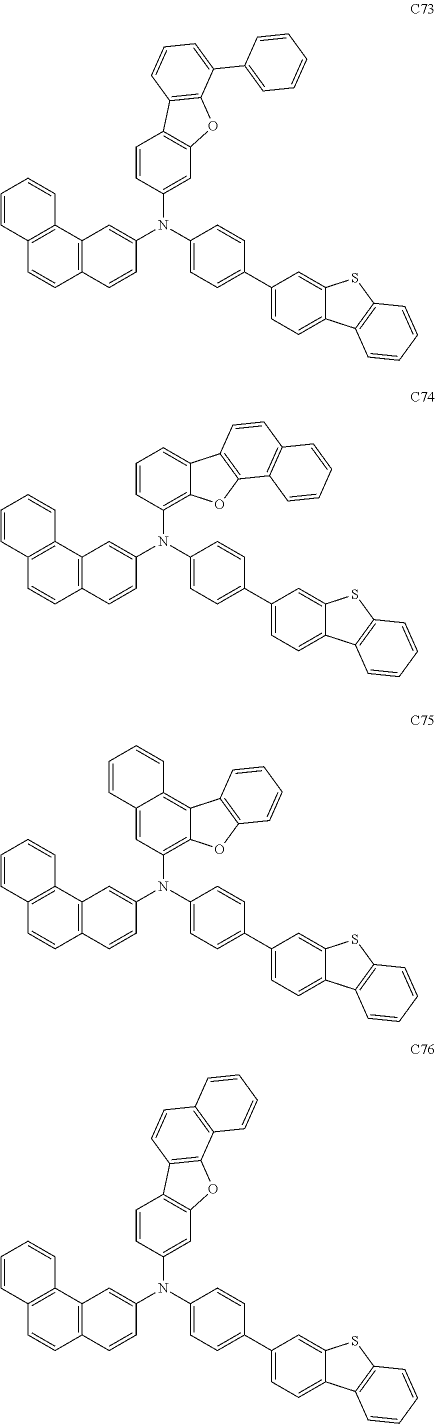 [SMILES: c1ccc(-c2cccc3c2oc2cc(N(c4ccc(-c5ccc6c(c5)sc5ccccc56)cc4)c4ccc5ccc6ccccc6c5c4)ccc23)cc1 ; c1ccc2c(c1)ccc1ccc(N(c3ccc(-c4ccc5c(c4)sc4ccccc45)cc3)c3cccc4c3oc3c5ccccc5ccc43)cc12 ; c1ccc2c(c1)ccc1ccc(N(c3ccc(-c4ccc5c(c4)sc4ccccc45)cc3)c3cc4ccccc4c4c3oc3ccccc34)cc12 ; c1ccc2c(c1)ccc1ccc(N(c3ccc(-c4ccc5c(c4)sc4ccccc45)cc3)c3ccc4c(c3)oc3c5ccccc5ccc43)cc12]

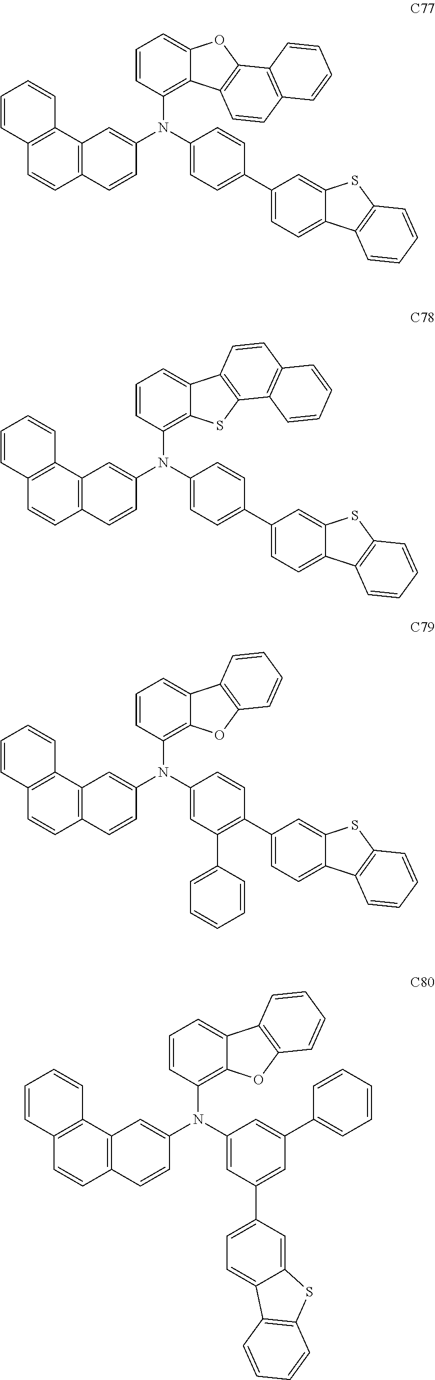 [SMILES: c1ccc2c(c1)ccc1ccc(N(c3ccc(-c4ccc5c(c4)sc4ccccc45)cc3)c3cccc4oc5c6ccccc6ccc5c34)cc12 ; c1ccc2c(c1)ccc1ccc(N(c3ccc(-c4ccc5c(c4)sc4ccccc45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2ccc3c(c2)sc2ccccc23)cc1 ; c1ccc(-c2cc(-c3ccc4c(c3)sc3ccccc34)cc(N(c3ccc4ccc5ccccc5c4c3)c3cccc4c3oc3ccccc34)c2)cc1]

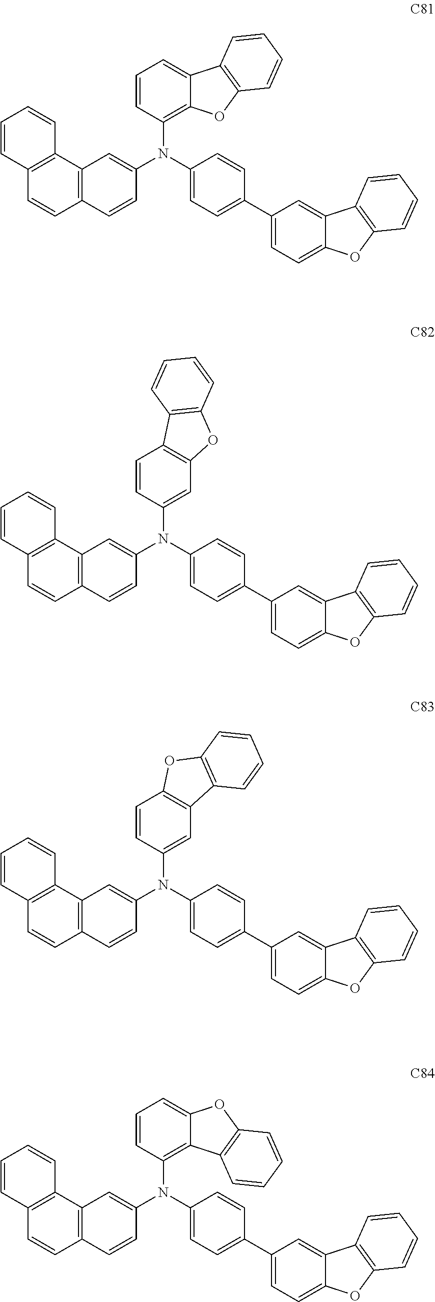 [SMILES: c1ccc2c(c1)ccc1ccc(N(c3ccc(-c4ccc5oc6ccccc6c5c4)cc3)c3cccc4c3oc3ccccc34)cc12 ; c1ccc2c(c1)ccc1ccc(N(c3ccc(-c4ccc5oc6ccccc6c5c4)cc3)c3ccc4c(c3)oc3ccccc34)cc12 ; c1ccc2c(c1)ccc1ccc(N(c3ccc(-c4ccc5oc6ccccc6c5c4)cc3)c3ccc4oc5ccccc5c4c3)cc12 ; c1ccc2c(c1)ccc1ccc(N(c3ccc(-c4ccc5oc6ccccc6c5c4)cc3)c3cccc4oc5ccccc5c34)cc12]

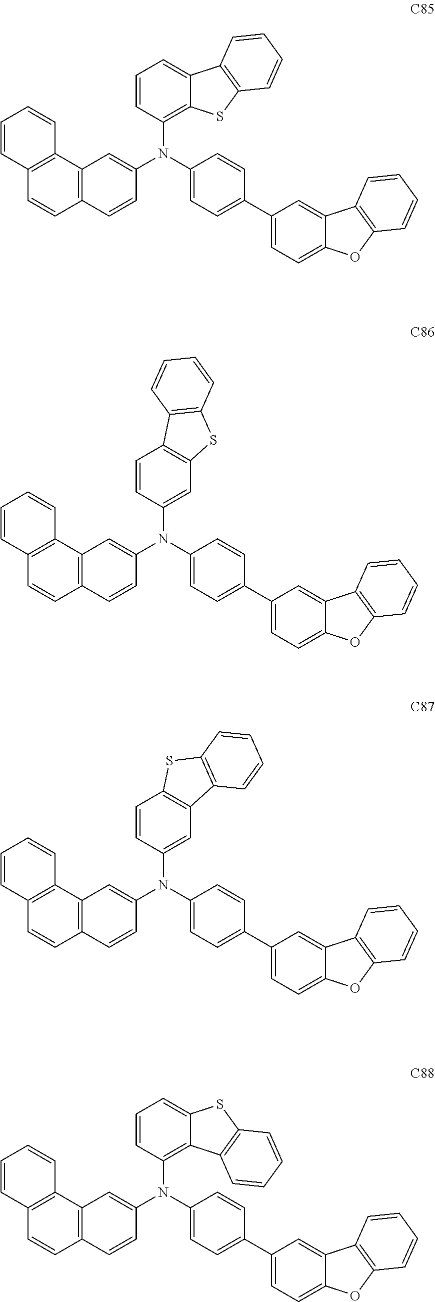 [SMILES: c1ccc2c(c1)ccc1ccc(N(c3ccc(-c4ccc5oc6ccccc6c5c4)cc3)c3cccc4c3sc3ccccc34)cc12 ; c1ccc2c(c1)ccc1ccc(N(c3ccc(-c4ccc5oc6ccccc6c5c4)cc3)c3ccc4c(c3)sc3ccccc34)cc12 ; c1ccc2c(c1)ccc1ccc(N(c3ccc(-c4ccc5oc6ccccc6c5c4)cc3)c3ccc4sc5ccccc5c4c3)cc12 ; c1ccc2c(c1)ccc1ccc(N(c3ccc(-c4ccc5oc6ccccc6c5c4)cc3)c3cccc4sc5ccccc5c34)cc12]

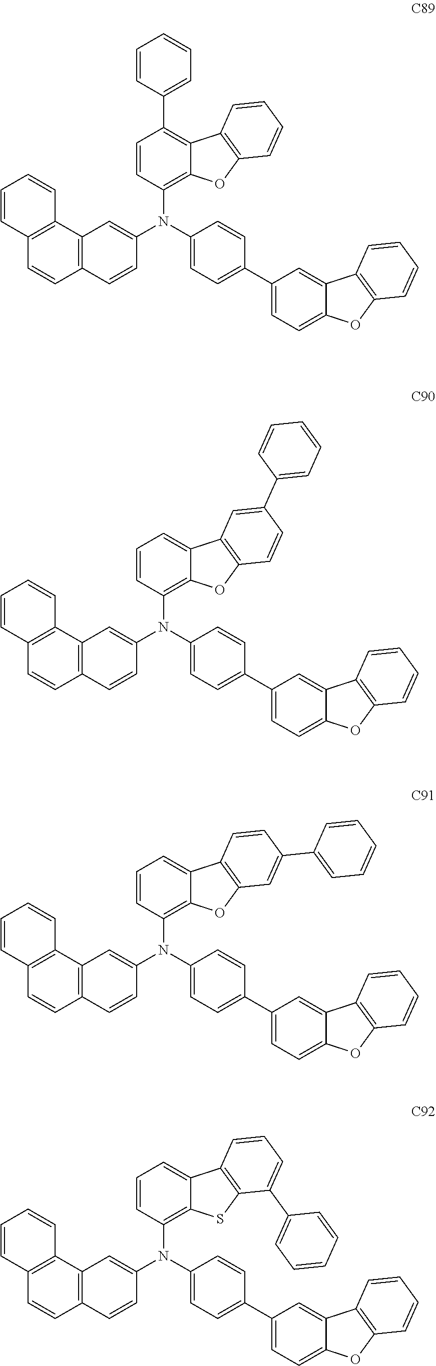 [SMILES: c1ccc(-c2ccc(N(c3ccc(-c4ccc5oc6ccccc6c5c4)cc3)c3ccc4ccc5ccccc5c4c3)c3oc4ccccc4c23)cc1 ; c1ccc(-c2ccc3oc4c(N(c5ccc(-c6ccc7oc8ccccc8c7c6)cc5)c5ccc6ccc7ccccc7c6c5)cccc4c3c2)cc1 ; c1ccc(-c2ccc3c(c2)oc2c(N(c4ccc(-c5ccc6oc7ccccc7c6c5)cc4)c4ccc5ccc6ccccc6c5c4)cccc23)cc1 ; c1ccc(-c2cccc3c2sc2c(N(c4ccc(-c5ccc6oc7ccccc7c6c5)cc4)c4ccc5ccc6ccccc6c5c4)cccc23)cc1]

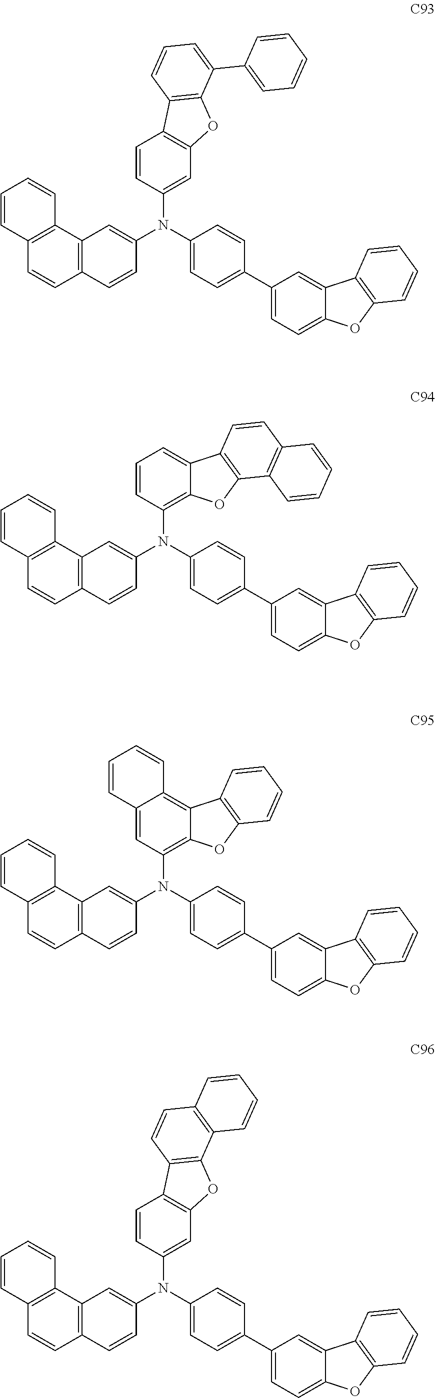 [SMILES: c1ccc(-c2cccc3c2oc2cc(N(c4ccc(-c5ccc6oc7ccccc7c6c5)cc4)c4ccc5ccc6ccccc6c5c4)ccc23)cc1 ; c1ccc2c(c1)ccc1ccc(N(c3ccc(-c4ccc5oc6ccccc6c5c4)cc3)c3cccc4c3oc3c5ccccc5ccc43)cc12 ; c1ccc2c(c1)ccc1ccc(N(c3ccc(-c4ccc5oc6ccccc6c5c4)cc3)c3cc4ccccc4c4c3oc3ccccc34)cc12 ; c1ccc2c(c1)ccc1ccc(N(c3ccc(-c4ccc5oc6ccccc6c5c4)cc3)c3ccc4c(c3)oc3c5ccccc5ccc43)cc12]

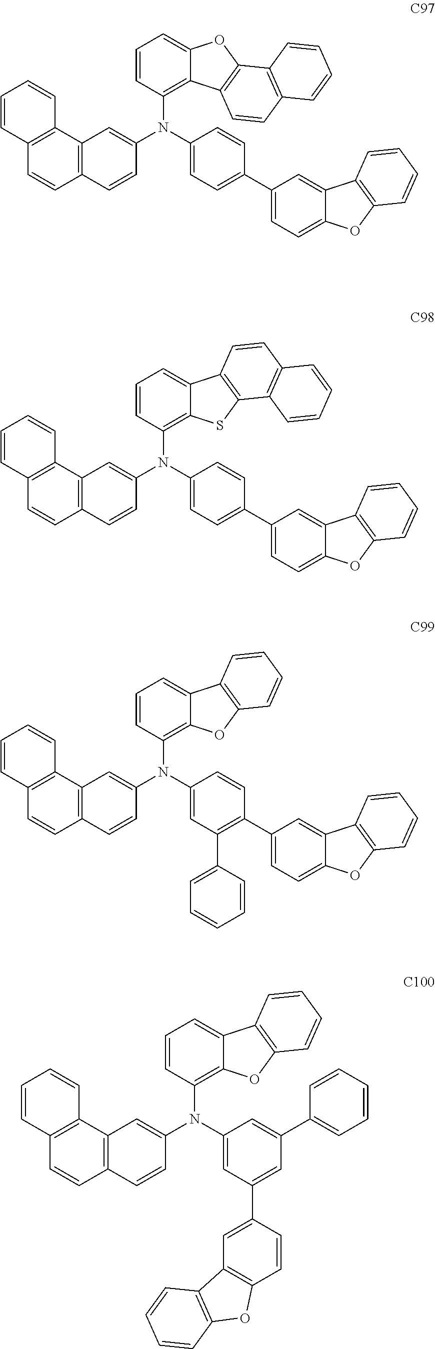 [SMILES: c1ccc2c(c1)ccc1ccc(N(c3ccc(-c4ccc5oc6ccccc6c5c4)cc3)c3cccc4oc5c6ccccc6ccc5c34)cc12 ; c1ccc2c(c1)ccc1ccc(N(c3ccc(-c4ccc5oc6ccccc6c5c4)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2ccc3oc4ccccc4c3c2)cc1 ; c1ccc(-c2cc(-c3ccc4oc5ccccc5c4c3)cc(N(c3ccc4ccc5ccccc5c4c3)c3cccc4c3oc3ccccc34)c2)cc1]

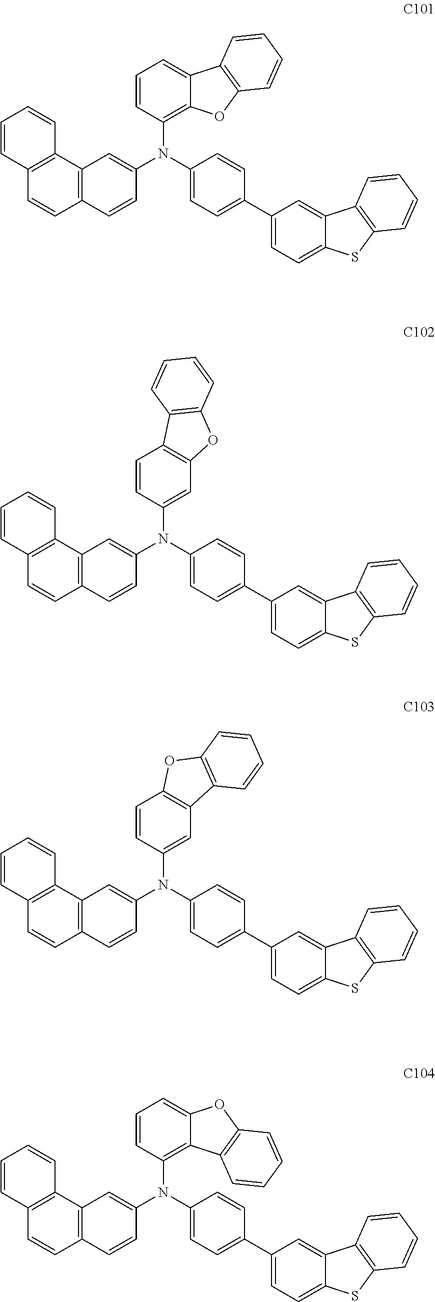 [SMILES: c1ccc2c(c1)ccc1ccc(N(c3ccc(-c4ccc5sc6ccccc6c5c4)cc3)c3cccc4c3oc3ccccc34)cc12 ; c1ccc2c(c1)ccc1ccc(N(c3ccc(-c4ccc5sc6ccccc6c5c4)cc3)c3ccc4c(c3)oc3ccccc34)cc12 ; c1ccc2c(c1)ccc1ccc(N(c3ccc(-c4ccc5sc6ccccc6c5c4)cc3)c3ccc4oc5ccccc5c4c3)cc12 ; c1ccc2c(c1)ccc1ccc(N(c3ccc(-c4ccc5sc6ccccc6c5c4)cc3)c3cccc4oc5ccccc5c34)cc12]

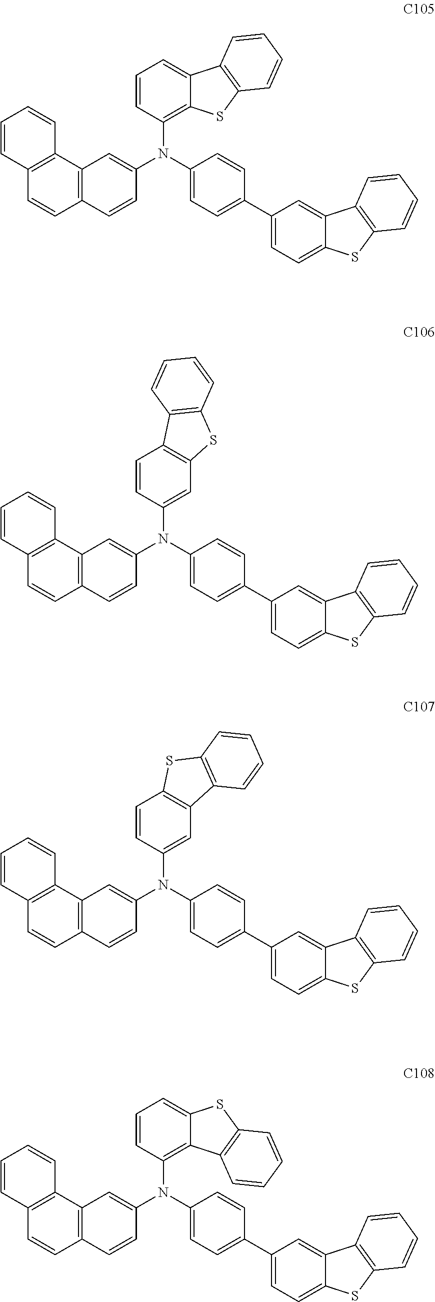 [SMILES: c1ccc2c(c1)ccc1ccc(N(c3ccc(-c4ccc5sc6ccccc6c5c4)cc3)c3cccc4c3sc3ccccc34)cc12 ; c1ccc2c(c1)ccc1ccc(N(c3ccc(-c4ccc5sc6ccccc6c5c4)cc3)c3ccc4c(c3)sc3ccccc34)cc12 ; c1ccc2c(c1)ccc1ccc(N(c3ccc(-c4ccc5sc6ccccc6c5c4)cc3)c3ccc4sc5ccccc5c4c3)cc12 ; c1ccc2c(c1)ccc1ccc(N(c3ccc(-c4ccc5sc6ccccc6c5c4)cc3)c3cccc4sc5ccccc5c34)cc12]

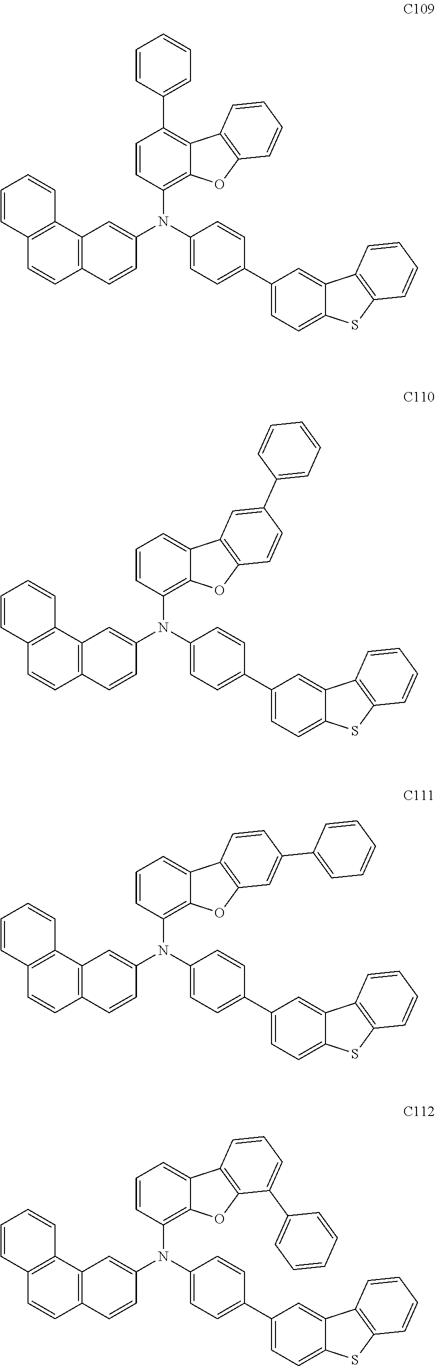 [SMILES: c1ccc(-c2ccc(N(c3ccc(-c4ccc5sc6ccccc6c5c4)cc3)c3ccc4ccc5ccccc5c4c3)c3oc4ccccc4c23)cc1 ; c1ccc(-c2ccc3oc4c(N(c5ccc(-c6ccc7sc8ccccc8c7c6)cc5)c5ccc6ccc7ccccc7c6c5)cccc4c3c2)cc1 ; c1ccc(-c2ccc3c(c2)oc2c(N(c4ccc(-c5ccc6sc7ccccc7c6c5)cc4)c4ccc5ccc6ccccc6c5c4)cccc23)cc1 ; c1ccc(-c2cccc3c2oc2c(N(c4ccc(-c5ccc6sc7ccccc7c6c5)cc4)c4ccc5ccc6ccccc6c5c4)cccc23)cc1]

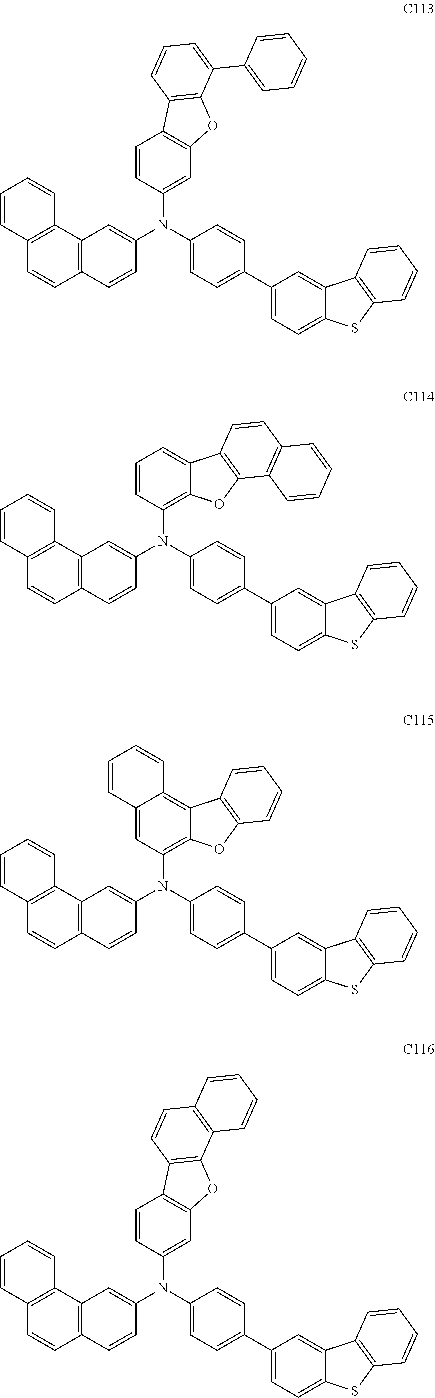 [SMILES: c1ccc(-c2cccc3c2oc2cc(N(c4ccc(-c5ccc6sc7ccccc7c6c5)cc4)c4ccc5ccc6ccccc6c5c4)ccc23)cc1 ; c1ccc2c(c1)ccc1ccc(N(c3ccc(-c4ccc5sc6ccccc6c5c4)cc3)c3cccc4c3oc3c5ccccc5ccc43)cc12 ; c1ccc2c(c1)ccc1ccc(N(c3ccc(-c4ccc5sc6ccccc6c5c4)cc3)c3cc4ccccc4c4c3oc3ccccc34)cc12 ; c1ccc2c(c1)ccc1ccc(N(c3ccc(-c4ccc5sc6ccccc6c5c4)cc3)c3ccc4c(c3)oc3c5ccccc5ccc43)cc12]

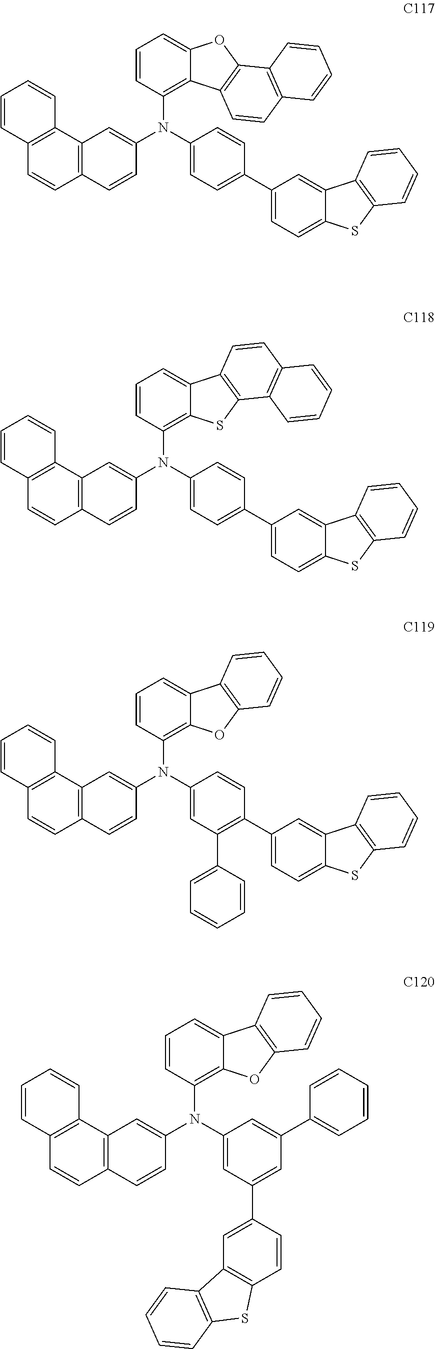 [SMILES: c1ccc2c(c1)ccc1ccc(N(c3ccc(-c4ccc5sc6ccccc6c5c4)cc3)c3cccc4oc5c6ccccc6ccc5c34)cc12 ; c1ccc2c(c1)ccc1ccc(N(c3ccc(-c4ccc5sc6ccccc6c5c4)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2ccc3sc4ccccc4c3c2)cc1 ; c1ccc(-c2cc(-c3ccc4sc5ccccc5c4c3)cc(N(c3ccc4ccc5ccccc5c4c3)c3cccc4c3oc3ccccc34)c2)cc1]

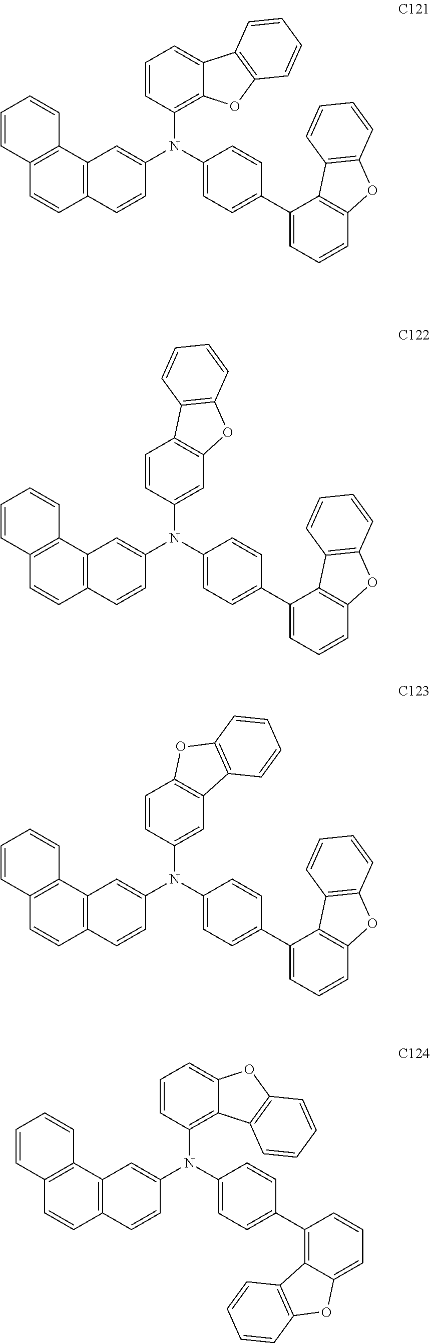 [SMILES: c1ccc2c(c1)ccc1ccc(N(c3ccc(-c4cccc5oc6ccccc6c45)cc3)c3cccc4c3oc3ccccc34)cc12 ; c1ccc2c(c1)ccc1ccc(N(c3ccc(-c4cccc5oc6ccccc6c45)cc3)c3ccc4c(c3)oc3ccccc34)cc12 ; c1ccc2c(c1)ccc1ccc(N(c3ccc(-c4cccc5oc6ccccc6c45)cc3)c3ccc4oc5ccccc5c4c3)cc12 ; c1ccc2c(c1)ccc1ccc(N(c3ccc(-c4cccc5oc6ccccc6c45)cc3)c3cccc4oc5ccccc5c34)cc12]

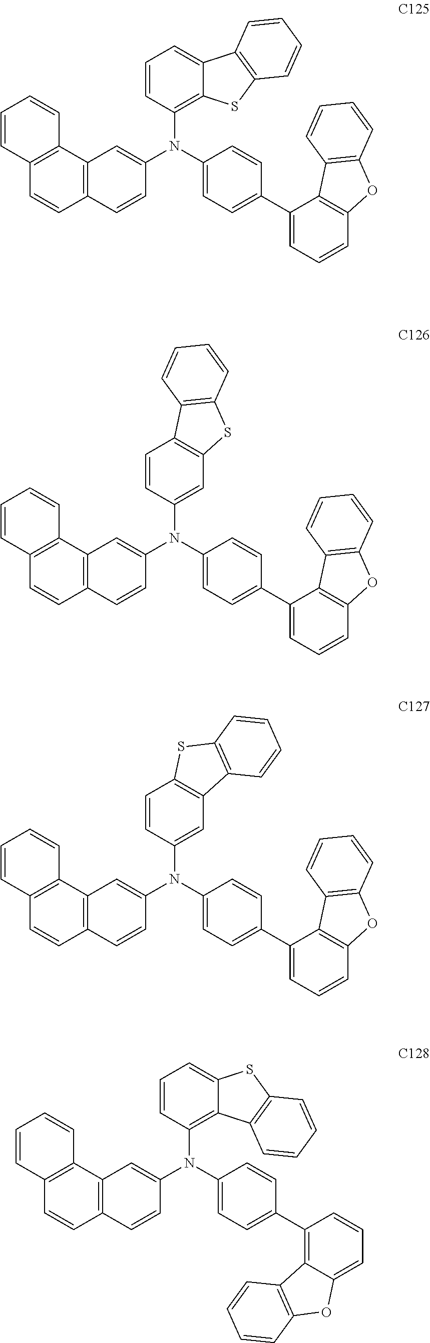 [SMILES: c1ccc2c(c1)ccc1ccc(N(c3ccc(-c4cccc5oc6ccccc6c45)cc3)c3cccc4c3sc3ccccc34)cc12 ; c1ccc2c(c1)ccc1ccc(N(c3ccc(-c4cccc5oc6ccccc6c45)cc3)c3ccc4c(c3)sc3ccccc34)cc12 ; c1ccc2c(c1)ccc1ccc(N(c3ccc(-c4cccc5oc6ccccc6c45)cc3)c3ccc4sc5ccccc5c4c3)cc12 ; c1ccc2c(c1)ccc1ccc(N(c3ccc(-c4cccc5oc6ccccc6c45)cc3)c3cccc4sc5ccccc5c34)cc12]

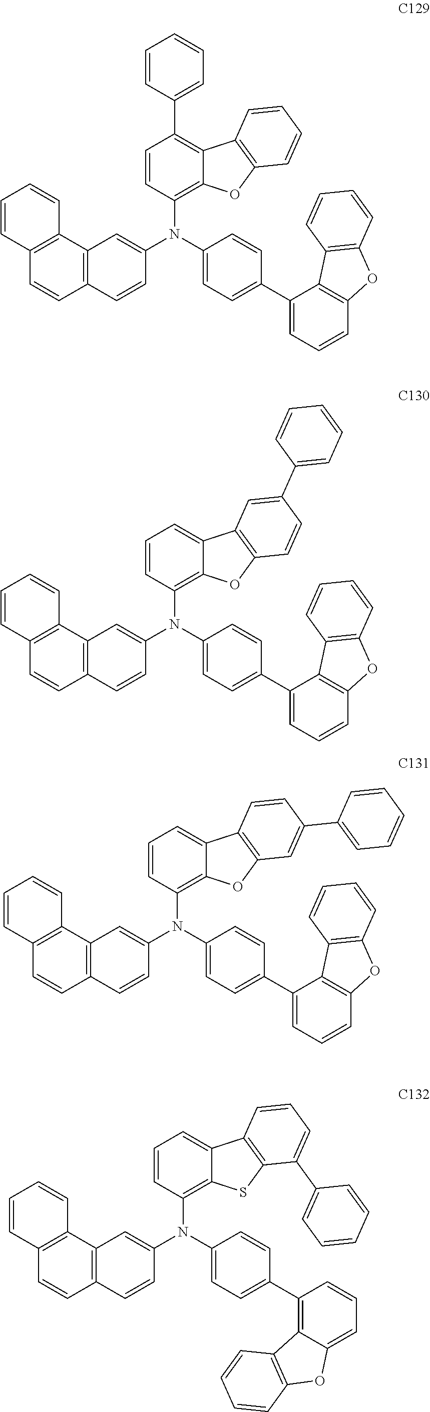 [SMILES: c1ccc(-c2ccc(N(c3ccc(-c4cccc5oc6ccccc6c45)cc3)c3ccc4ccc5ccccc5c4c3)c3oc4ccccc4c23)cc1 ; c1ccc(-c2ccc3oc4c(N(c5ccc(-c6cccc7oc8ccccc8c67)cc5)c5ccc6ccc7ccccc7c6c5)cccc4c3c2)cc1 ; c1ccc(-c2ccc3c(c2)oc2c(N(c4ccc(-c5cccc6oc7ccccc7c56)cc4)c4ccc5ccc6ccccc6c5c4)cccc23)cc1 ; c1ccc(-c2cccc3c2sc2c(N(c4ccc(-c5cccc6oc7ccccc7c56)cc4)c4ccc5ccc6ccccc6c5c4)cccc23)cc1]

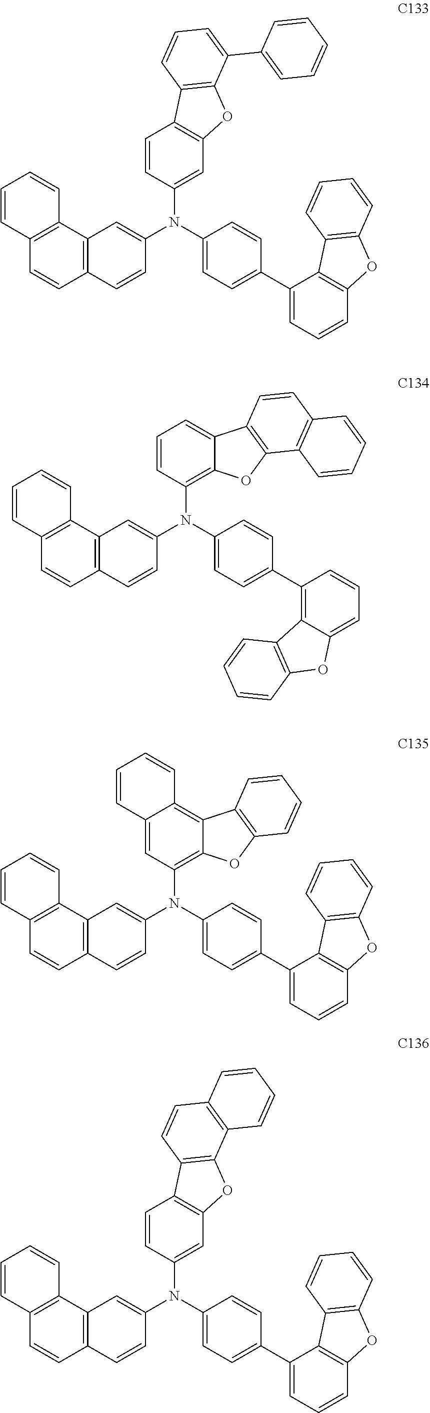 [SMILES: c1ccc(-c2cccc3c2oc2cc(N(c4ccc(-c5cccc6oc7ccccc7c56)cc4)c4ccc5ccc6ccccc6c5c4)ccc23)cc1 ; c1ccc2c(c1)ccc1ccc(N(c3ccc(-c4cccc5oc6ccccc6c45)cc3)c3cccc4c3oc3c5ccccc5ccc43)cc12 ; c1ccc2c(c1)ccc1ccc(N(c3ccc(-c4cccc5oc6ccccc6c45)cc3)c3cc4ccccc4c4c3oc3ccccc34)cc12 ; c1ccc2c(c1)ccc1ccc(N(c3ccc(-c4cccc5oc6ccccc6c45)cc3)c3ccc4c(c3)oc3c5ccccc5ccc43)cc12]

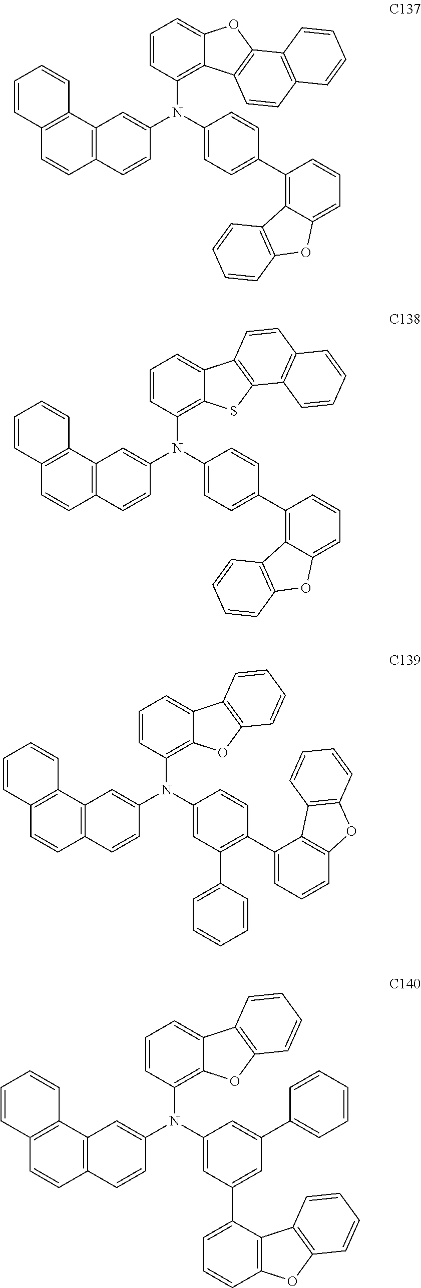 [SMILES: c1ccc2c(c1)ccc1ccc(N(c3ccc(-c4cccc5oc6ccccc6c45)cc3)c3cccc4oc5c6ccccc6ccc5c34)cc12 ; c1ccc2c(c1)ccc1ccc(N(c3ccc(-c4cccc5oc6ccccc6c45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2cccc3oc4ccccc4c23)cc1 ; c1ccc(-c2cc(-c3cccc4oc5ccccc5c34)cc(N(c3ccc4ccc5ccccc5c4c3)c3cccc4c3oc3ccccc34)c2)cc1]

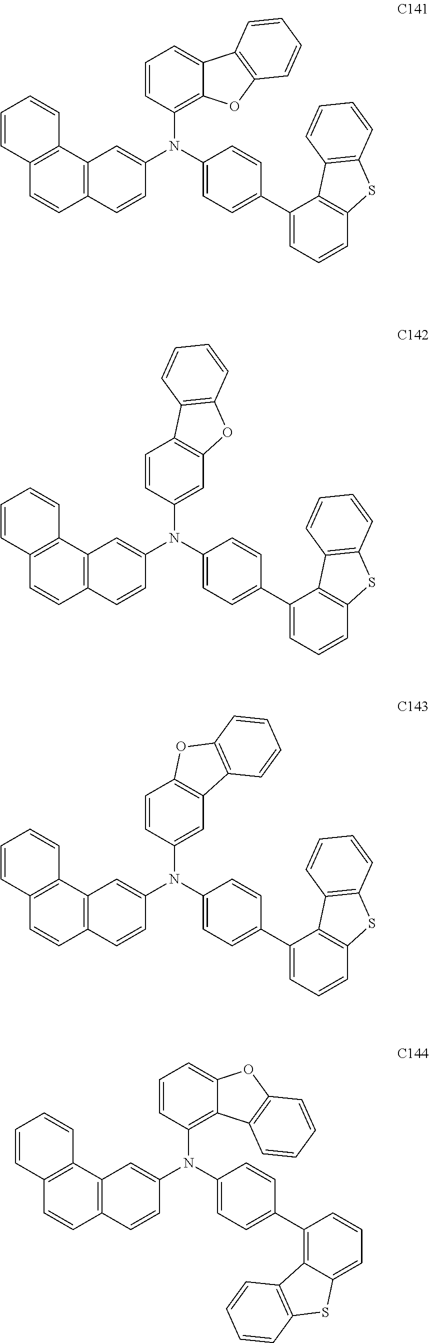 [SMILES: c1ccc2c(c1)ccc1ccc(N(c3ccc(-c4cccc5sc6ccccc6c45)cc3)c3cccc4c3oc3ccccc34)cc12 ; c1ccc2c(c1)ccc1ccc(N(c3ccc(-c4cccc5sc6ccccc6c45)cc3)c3ccc4c(c3)oc3ccccc34)cc12 ; c1ccc2c(c1)ccc1ccc(N(c3ccc(-c4cccc5sc6ccccc6c45)cc3)c3ccc4oc5ccccc5c4c3)cc12 ; c1ccc2c(c1)ccc1ccc(N(c3ccc(-c4cccc5sc6ccccc6c45)cc3)c3cccc4oc5ccccc5c34)cc12]

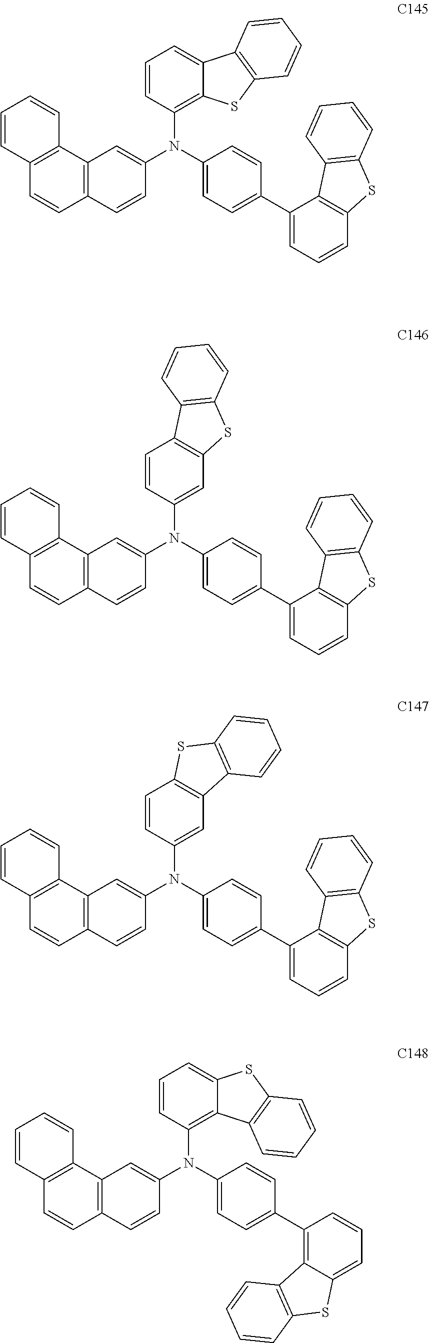 [SMILES: c1ccc2c(c1)ccc1ccc(N(c3ccc(-c4cccc5sc6ccccc6c45)cc3)c3cccc4c3sc3ccccc34)cc12 ; c1ccc2c(c1)ccc1ccc(N(c3ccc(-c4cccc5sc6ccccc6c45)cc3)c3ccc4c(c3)sc3ccccc34)cc12 ; c1ccc2c(c1)ccc1ccc(N(c3ccc(-c4cccc5sc6ccccc6c45)cc3)c3ccc4sc5ccccc5c4c3)cc12 ; c1ccc2c(c1)ccc1ccc(N(c3ccc(-c4cccc5sc6ccccc6c45)cc3)c3cccc4sc5ccccc5c34)cc12]

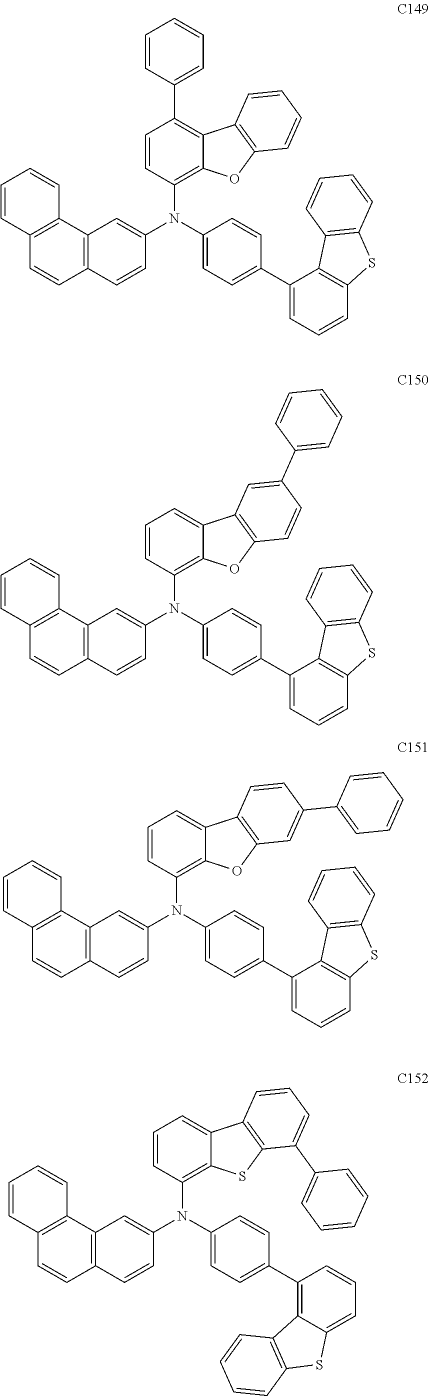 [SMILES: c1ccc(-c2ccc(N(c3ccc(-c4cccc5sc6ccccc6c45)cc3)c3ccc4ccc5ccccc5c4c3)c3oc4ccccc4c23)cc1 ; c1ccc(-c2ccc3oc4c(N(c5ccc(-c6cccc7sc8ccccc8c67)cc5)c5ccc6ccc7ccccc7c6c5)cccc4c3c2)cc1 ; c1ccc(-c2ccc3c(c2)oc2c(N(c4ccc(-c5cccc6sc7ccccc7c56)cc4)c4ccc5ccc6ccccc6c5c4)cccc23)cc1 ; c1ccc(-c2cccc3c2sc2c(N(c4ccc(-c5cccc6sc7ccccc7c56)cc4)c4ccc5ccc6ccccc6c5c4)cccc23)cc1]

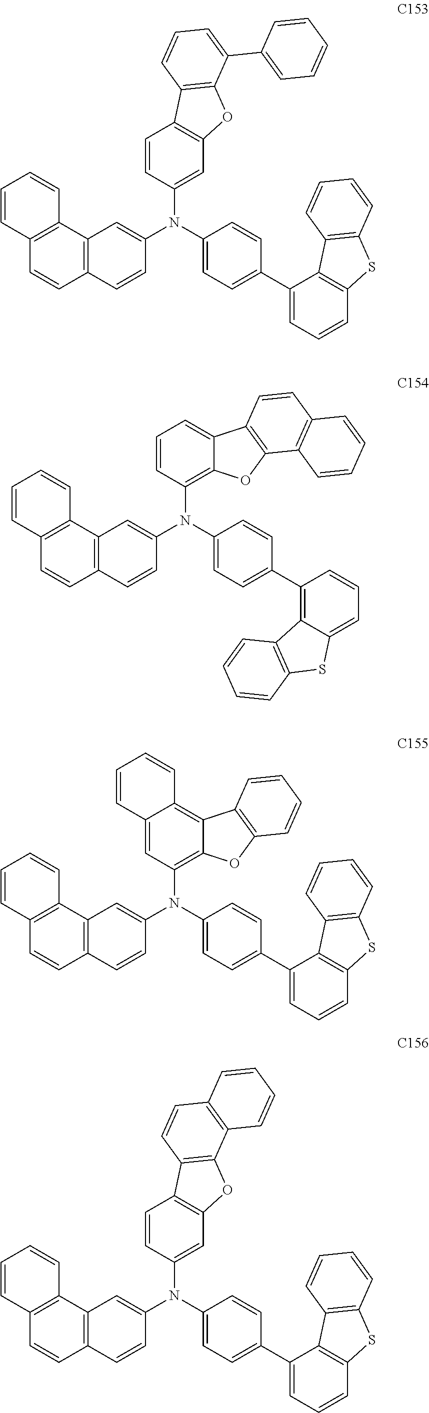 [SMILES: c1ccc(-c2cccc3c2oc2cc(N(c4ccc(-c5cccc6sc7ccccc7c56)cc4)c4ccc5ccc6ccccc6c5c4)ccc23)cc1 ; c1ccc2c(c1)ccc1ccc(N(c3ccc(-c4cccc5sc6ccccc6c45)cc3)c3cccc4c3oc3c5ccccc5ccc43)cc12 ; c1ccc2c(c1)ccc1ccc(N(c3ccc(-c4cccc5sc6ccccc6c45)cc3)c3cc4ccccc4c4c3oc3ccccc34)cc12 ; c1ccc2c(c1)ccc1ccc(N(c3ccc(-c4cccc5sc6ccccc6c45)cc3)c3ccc4c(c3)oc3c5ccccc5ccc43)cc12]

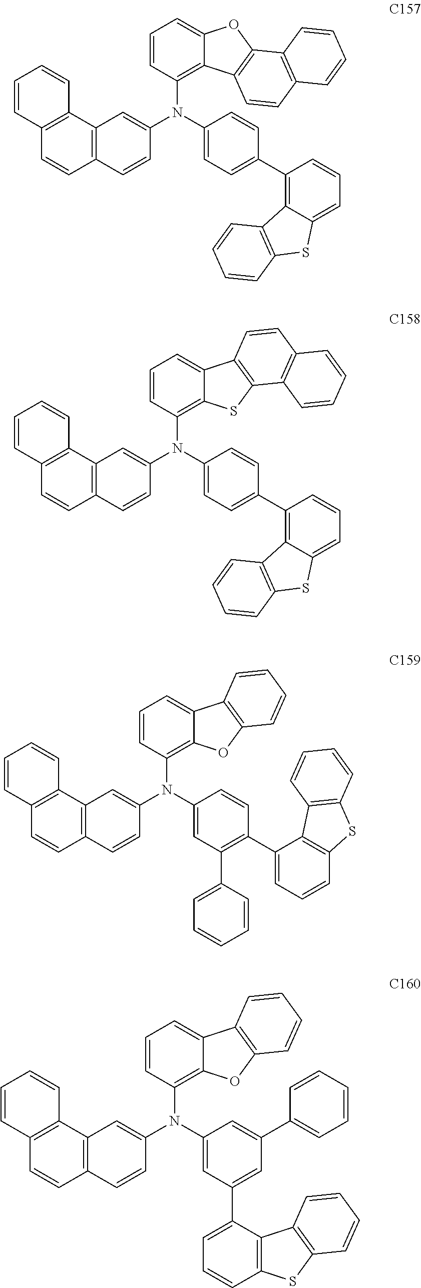 [SMILES: c1ccc2c(c1)ccc1ccc(N(c3ccc(-c4cccc5sc6ccccc6c45)cc3)c3cccc4oc5c6ccccc6ccc5c34)cc12 ; c1ccc2c(c1)ccc1ccc(N(c3ccc(-c4cccc5sc6ccccc6c45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2cccc3sc4ccccc4c23)cc1 ; c1ccc(-c2cc(-c3cccc4sc5ccccc5c34)cc(N(c3ccc4ccc5ccccc5c4c3)c3cccc4c3oc3ccccc34)c2)cc1]

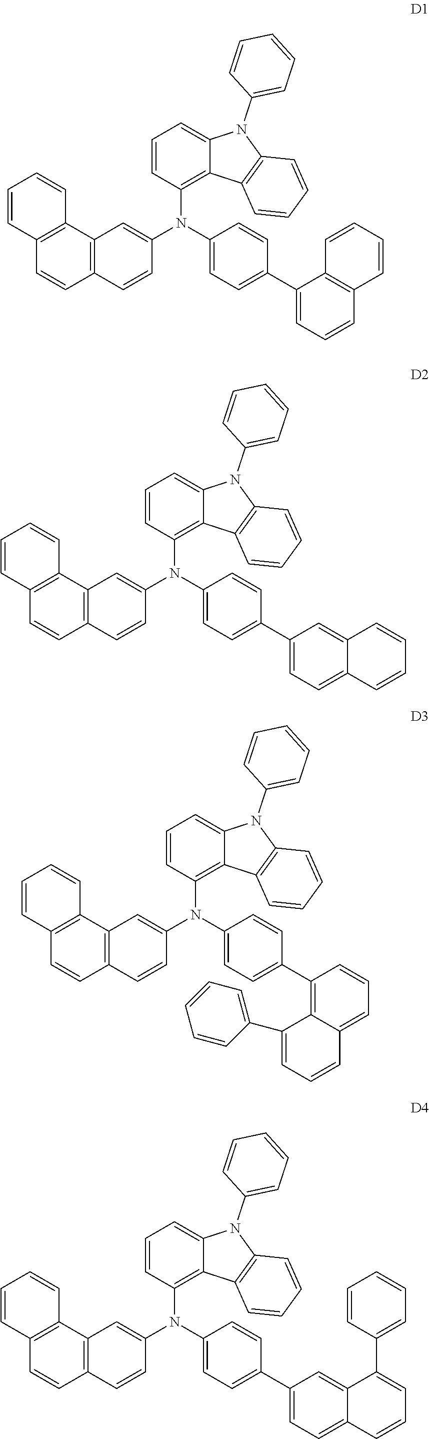 [SMILES: c1ccc(-n2c3ccccc3c3c(N(c4ccc(-c5cccc6ccccc56)cc4)c4ccc5ccc6ccccc6c5c4)cccc32)cc1 ; c1ccc(-n2c3ccccc3c3c(N(c4ccc(-c5ccc6ccccc6c5)cc4)c4ccc5ccc6ccccc6c5c4)cccc32)cc1 ; c1ccc(-c2cccc3cccc(-c4ccc(N(c5ccc6ccc7ccccc7c6c5)c5cccc6c5c5ccccc5n6-c5ccccc5)cc4)c23)cc1 ; c1ccc(-c2cccc3ccc(-c4ccc(N(c5ccc6ccc7ccccc7c6c5)c5cccc6c5c5ccccc5n6-c5ccccc5)cc4)cc23)cc1]

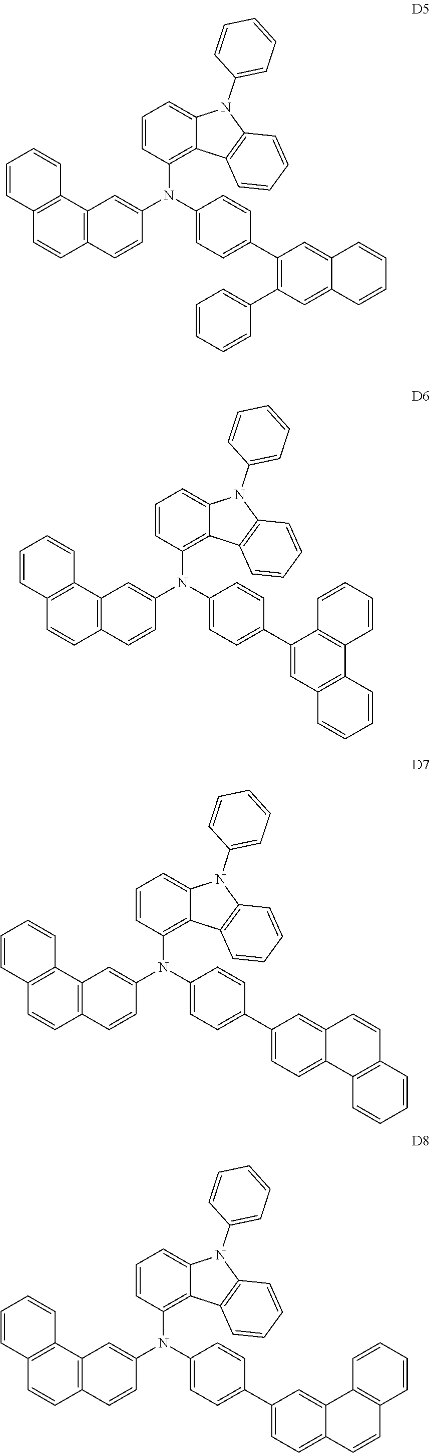 [SMILES: c1ccc(-c2cc3ccccc3cc2-c2ccc(N(c3ccc4ccc5ccccc5c4c3)c3cccc4c3c3ccccc3n4-c3ccccc3)cc2)cc1 ; c1ccc(-n2c3ccccc3c3c(N(c4ccc(-c5cc6ccccc6c6ccccc56)cc4)c4ccc5ccc6ccccc6c5c4)cccc32)cc1 ; c1ccc(-n2c3ccccc3c3c(N(c4ccc(-c5ccc6c(ccc7ccccc76)c5)cc4)c4ccc5ccc6ccccc6c5c4)cccc32)cc1 ; c1ccc(-n2c3ccccc3c3c(N(c4ccc(-c5ccc6ccc7ccccc7c6c5)cc4)c4ccc5ccc6ccccc6c5c4)cccc32)cc1]

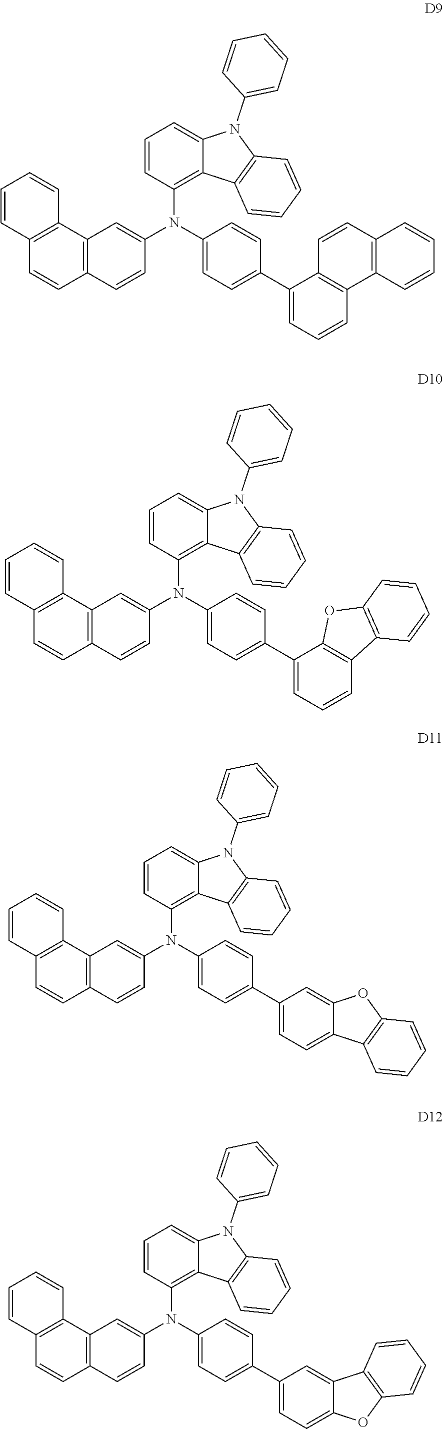 [SMILES: c1ccc(-n2c3ccccc3c3c(N(c4ccc(-c5cccc6c5ccc5ccccc56)cc4)c4ccc5ccc6ccccc6c5c4)cccc32)cc1 ; c1ccc(-n2c3ccccc3c3c(N(c4ccc(-c5cccc6c5oc5ccccc56)cc4)c4ccc5ccc6ccccc6c5c4)cccc32)cc1 ; c1ccc(-n2c3ccccc3c3c(N(c4ccc(-c5ccc6c(c5)oc5ccccc56)cc4)c4ccc5ccc6ccccc6c5c4)cccc32)cc1 ; c1ccc(-n2c3ccccc3c3c(N(c4ccc(-c5ccc6oc7ccccc7c6c5)cc4)c4ccc5ccc6ccccc6c5c4)cccc32)cc1]

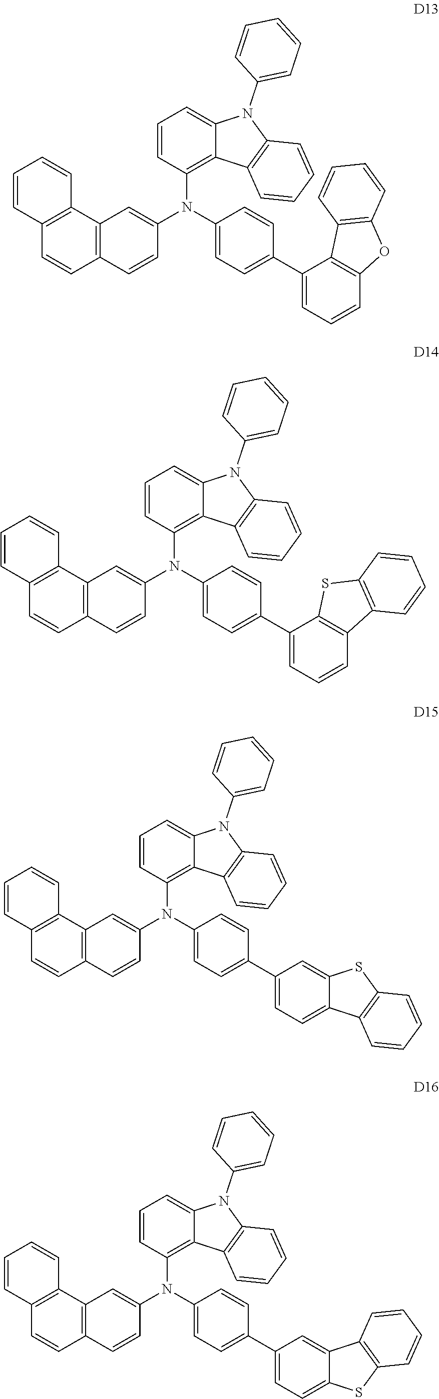 [SMILES: c1ccc(-n2c3ccccc3c3c(N(c4ccc(-c5cccc6oc7ccccc7c56)cc4)c4ccc5ccc6ccccc6c5c4)cccc32)cc1 ; c1ccc(-n2c3ccccc3c3c(N(c4ccc(-c5cccc6c5sc5ccccc56)cc4)c4ccc5ccc6ccccc6c5c4)cccc32)cc1 ; c1ccc(-n2c3ccccc3c3c(N(c4ccc(-c5ccc6c(c5)sc5ccccc56)cc4)c4ccc5ccc6ccccc6c5c4)cccc32)cc1 ; c1ccc(-n2c3ccccc3c3c(N(c4ccc(-c5ccc6sc7ccccc7c6c5)cc4)c4ccc5ccc6ccccc6c5c4)cccc32)cc1]

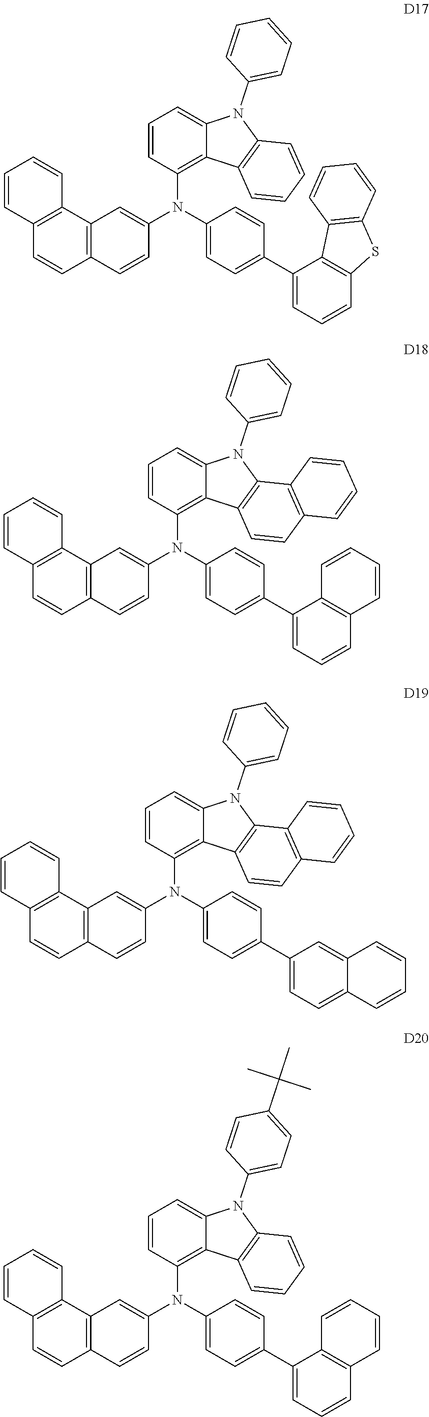 [SMILES: c1ccc(-n2c3ccccc3c3c(N(c4ccc(-c5cccc6sc7ccccc7c56)cc4)c4ccc5ccc6ccccc6c5c4)cccc32)cc1 ; c1ccc(-n2c3cccc(N(c4ccc(-c5cccc6ccccc56)cc4)c4ccc5ccc6ccccc6c5c4)c3c3ccc4ccccc4c32)cc1 ; c1ccc(-n2c3cccc(N(c4ccc(-c5ccc6ccccc6c5)cc4)c4ccc5ccc6ccccc6c5c4)c3c3ccc4ccccc4c32)cc1 ; CC(C)(C)c1ccc(-n2c3ccccc3c3c(N(c4ccc(-c5cccc6ccccc56)cc4)c4ccc5ccc6ccccc6c5c4)cccc32)cc1]

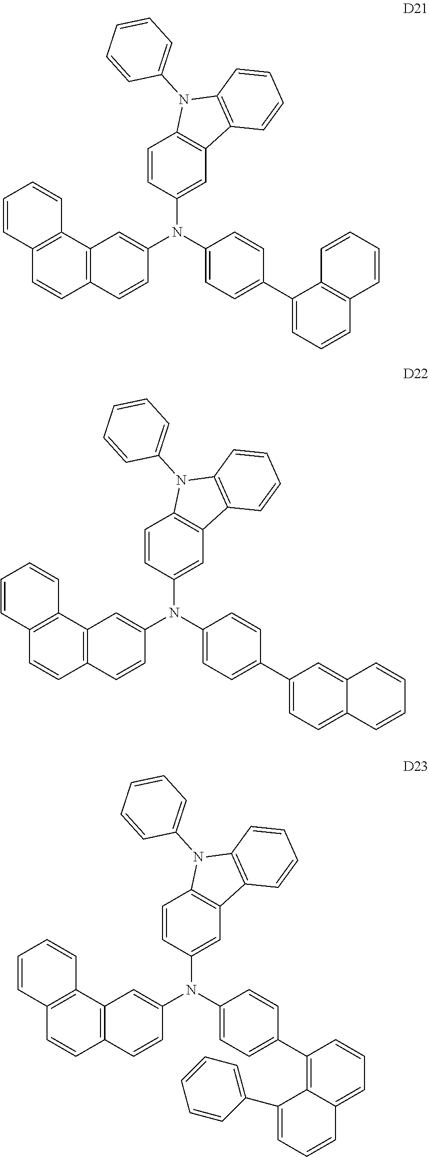 [SMILES: c1ccc(-n2c3ccccc3c3cc(N(c4ccc(-c5cccc6ccccc56)cc4)c4ccc5ccc6ccccc6c5c4)ccc32)cc1 ; c1ccc(-n2c3ccccc3c3cc(N(c4ccc(-c5ccc6ccccc6c5)cc4)c4ccc5ccc6ccccc6c5c4)ccc32)cc1 ; c1ccc(-c2cccc3cccc(-c4ccc(N(c5ccc6ccc7ccccc7c6c5)c5ccc6c(c5)c5ccccc5n6-c5ccccc5)cc4)c23)cc1]

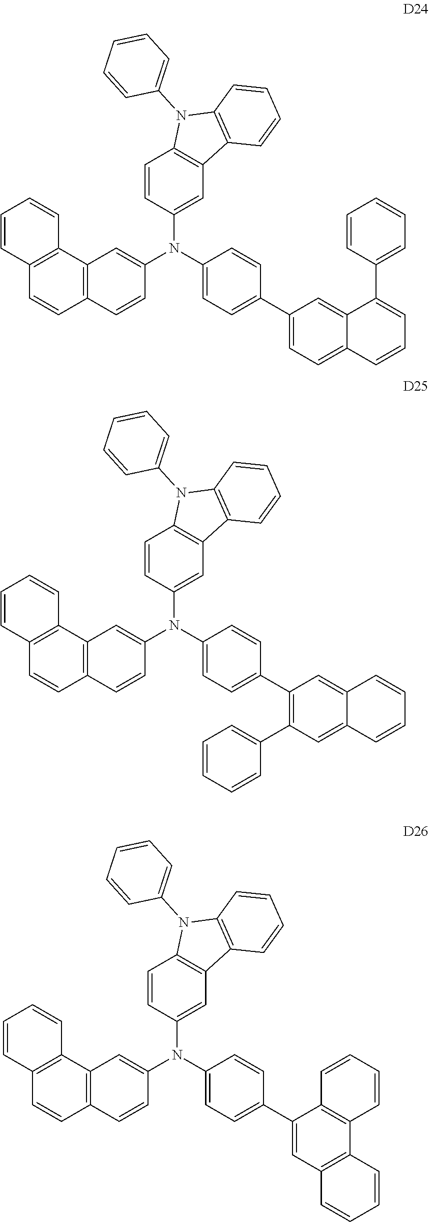 [SMILES: c1ccc(-c2cccc3ccc(-c4ccc(N(c5ccc6ccc7ccccc7c6c5)c5ccc6c(c5)c5ccccc5n6-c5ccccc5)cc4)cc23)cc1 ; c1ccc(-c2cc3ccccc3cc2-c2ccc(N(c3ccc4ccc5ccccc5c4c3)c3ccc4c(c3)c3ccccc3n4-c3ccccc3)cc2)cc1 ; c1ccc(-n2c3ccccc3c3cc(N(c4ccc(-c5cc6ccccc6c6ccccc56)cc4)c4ccc5ccc6ccccc6c5c4)ccc32)cc1]

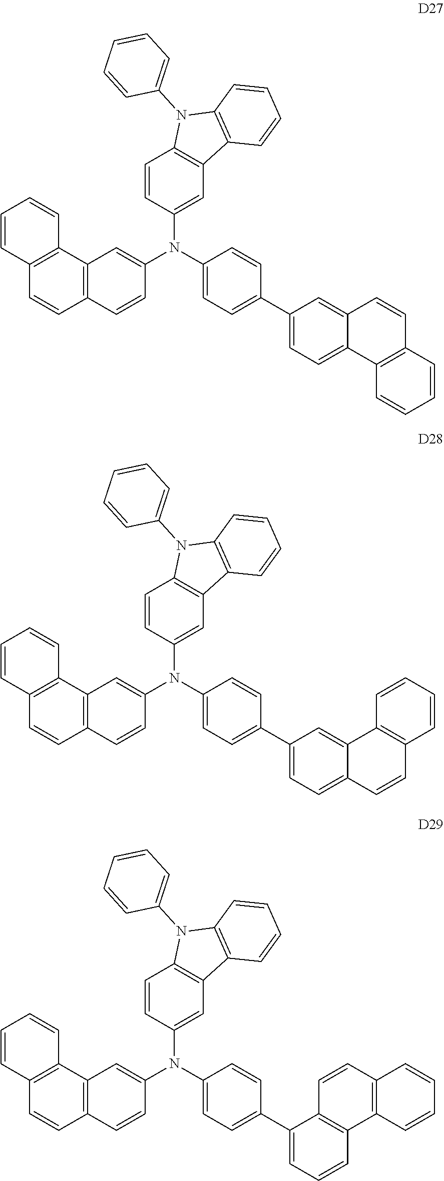 [SMILES: c1ccc(-n2c3ccccc3c3cc(N(c4ccc(-c5ccc6c(ccc7ccccc76)c5)cc4)c4ccc5ccc6ccccc6c5c4)ccc32)cc1 ; c1ccc(-n2c3ccccc3c3cc(N(c4ccc(-c5ccc6ccc7ccccc7c6c5)cc4)c4ccc5ccc6ccccc6c5c4)ccc32)cc1 ; c1ccc(-n2c3ccccc3c3cc(N(c4ccc(-c5cccc6c5ccc5ccccc56)cc4)c4ccc5ccc6ccccc6c5c4)ccc32)cc1]

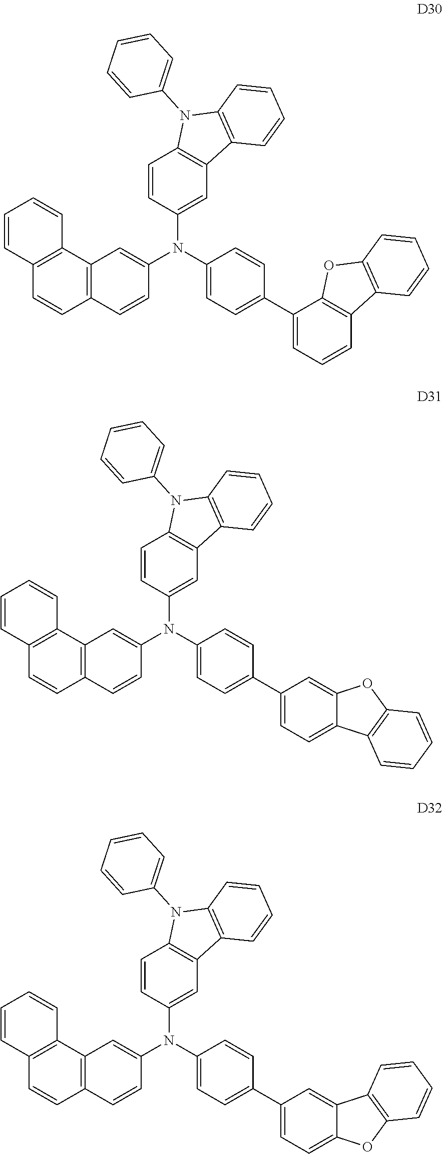 [SMILES: c1ccc(-n2c3ccccc3c3cc(N(c4ccc(-c5cccc6c5oc5ccccc56)cc4)c4ccc5ccc6ccccc6c5c4)ccc32)cc1 ; c1ccc(-n2c3ccccc3c3cc(N(c4ccc(-c5ccc6c(c5)oc5ccccc56)cc4)c4ccc5ccc6ccccc6c5c4)ccc32)cc1 ; c1ccc(-n2c3ccccc3c3cc(N(c4ccc(-c5ccc6oc7ccccc7c6c5)cc4)c4ccc5ccc6ccccc6c5c4)ccc32)cc1]

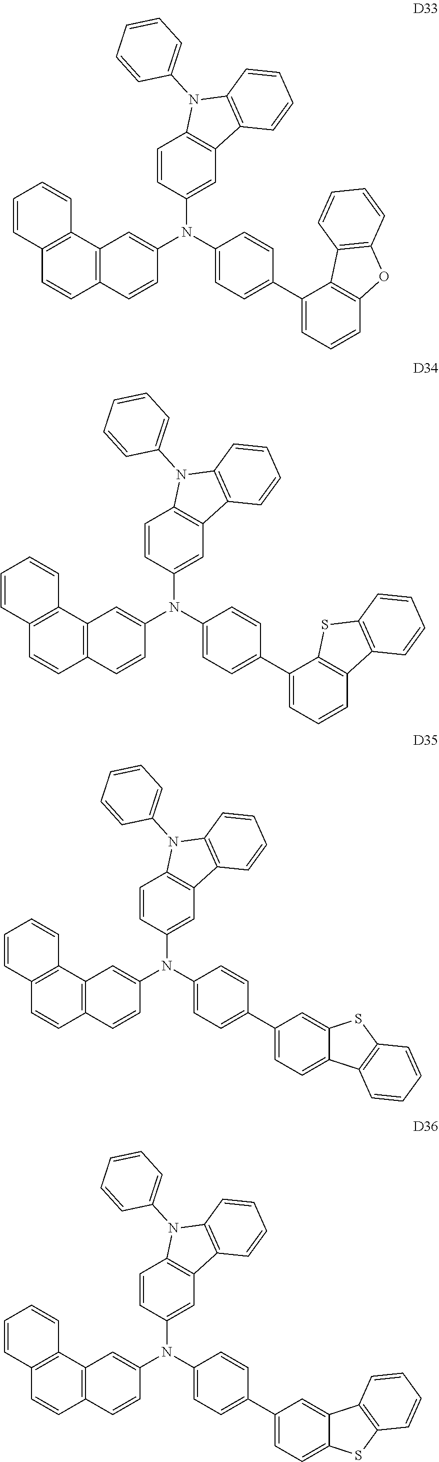 [SMILES: c1ccc(-n2c3ccccc3c3cc(N(c4ccc(-c5cccc6oc7ccccc7c56)cc4)c4ccc5ccc6ccccc6c5c4)ccc32)cc1 ; c1ccc(-n2c3ccccc3c3cc(N(c4ccc(-c5cccc6c5sc5ccccc56)cc4)c4ccc5ccc6ccccc6c5c4)ccc32)cc1 ; c1ccc(-n2c3ccccc3c3cc(N(c4ccc(-c5ccc6c(c5)sc5ccccc56)cc4)c4ccc5ccc6ccccc6c5c4)ccc32)cc1 ; c1ccc(-n2c3ccccc3c3cc(N(c4ccc(-c5ccc6sc7ccccc7c6c5)cc4)c4ccc5ccc6ccccc6c5c4)ccc32)cc1]

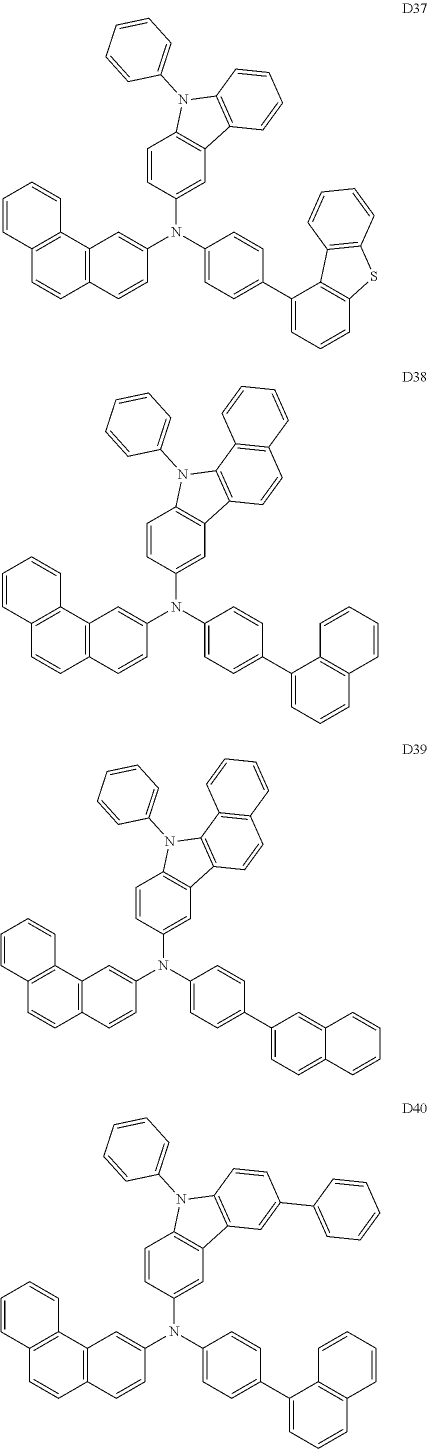 [SMILES: c1ccc(-n2c3ccccc3c3cc(N(c4ccc(-c5cccc6sc7ccccc7c56)cc4)c4ccc5ccc6ccccc6c5c4)ccc32)cc1 ; c1ccc(-n2c3ccc(N(c4ccc(-c5cccc6ccccc56)cc4)c4ccc5ccc6ccccc6c5c4)cc3c3ccc4ccccc4c32)cc1 ; c1ccc(-n2c3ccc(N(c4ccc(-c5ccc6ccccc6c5)cc4)c4ccc5ccc6ccccc6c5c4)cc3c3ccc4ccccc4c32)cc1 ; c1ccc(-c2ccc3c(c2)c2cc(N(c4ccc(-c5cccc6ccccc56)cc4)c4ccc5ccc6ccccc6c5c4)ccc2n3-c2ccccc2)cc1]

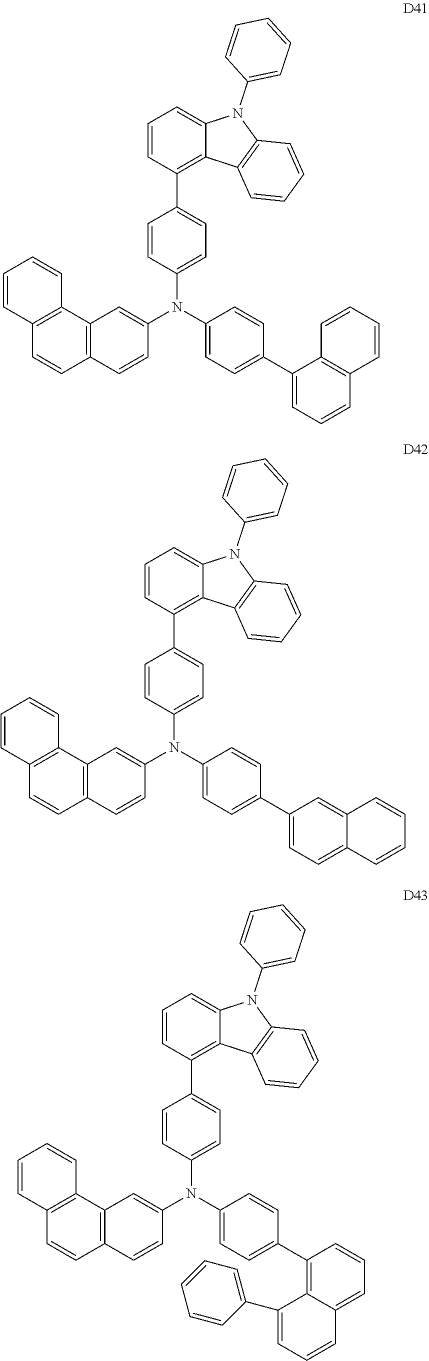 [SMILES: c1ccc(-n2c3ccccc3c3c(-c4ccc(N(c5ccc(-c6cccc7ccccc67)cc5)c5ccc6ccc7ccccc7c6c5)cc4)cccc32)cc1 ; c1ccc(-n2c3ccccc3c3c(-c4ccc(N(c5ccc(-c6ccc7ccccc7c6)cc5)c5ccc6ccc7ccccc7c6c5)cc4)cccc32)cc1 ; c1ccc(-c2cccc3cccc(-c4ccc(N(c5ccc(-c6cccc7c6c6ccccc6n7-c6ccccc6)cc5)c5ccc6ccc7ccccc7c6c5)cc4)c23)cc1]

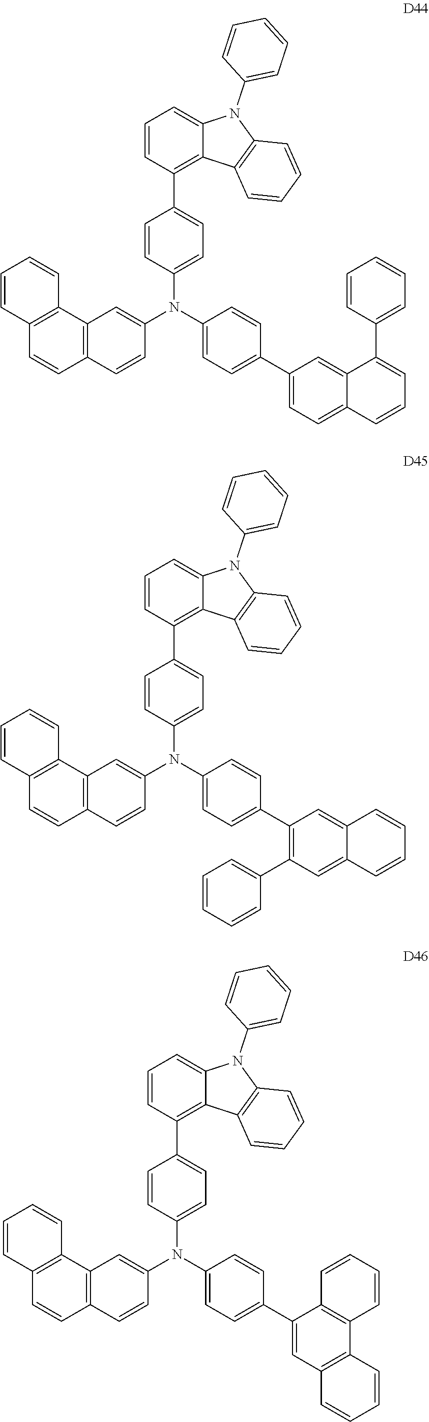 [SMILES: c1ccc(-c2cccc3ccc(-c4ccc(N(c5ccc(-c6cccc7c6c6ccccc6n7-c6ccccc6)cc5)c5ccc6ccc7ccccc7c6c5)cc4)cc23)cc1 ; c1ccc(-c2cc3ccccc3cc2-c2ccc(N(c3ccc(-c4cccc5c4c4ccccc4n5-c4ccccc4)cc3)c3ccc4ccc5ccccc5c4c3)cc2)cc1 ; c1ccc(-n2c3ccccc3c3c(-c4ccc(N(c5ccc(-c6cc7ccccc7c7ccccc67)cc5)c5ccc6ccc7ccccc7c6c5)cc4)cccc32)cc1]

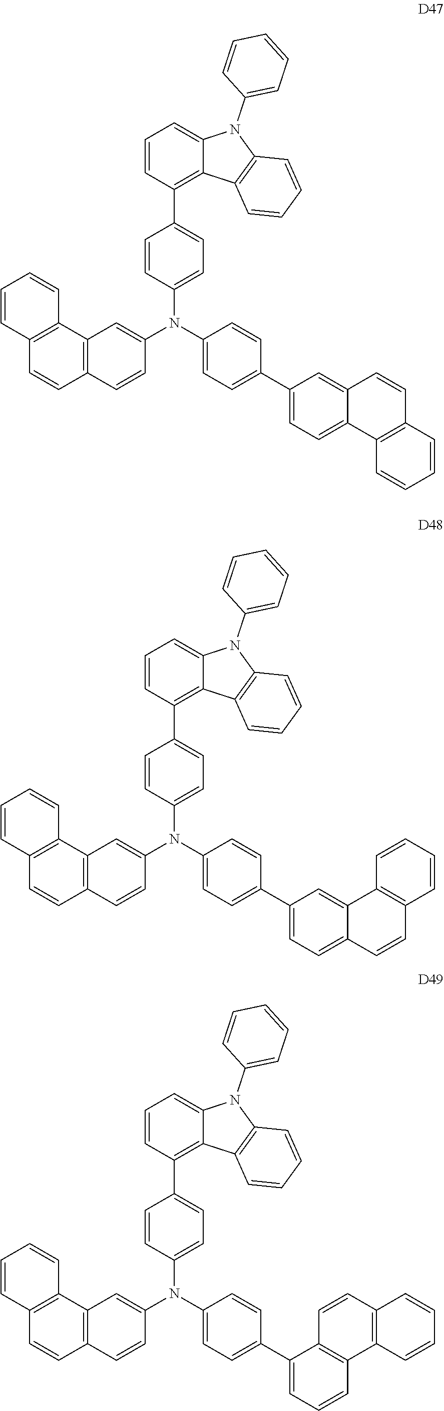 [SMILES: c1ccc(-n2c3ccccc3c3c(-c4ccc(N(c5ccc(-c6ccc7c(ccc8ccccc87)c6)cc5)c5ccc6ccc7ccccc7c6c5)cc4)cccc32)cc1 ; c1ccc(-n2c3ccccc3c3c(-c4ccc(N(c5ccc(-c6ccc7ccc8ccccc8c7c6)cc5)c5ccc6ccc7ccccc7c6c5)cc4)cccc32)cc1 ; c1ccc(-n2c3ccccc3c3c(-c4ccc(N(c5ccc(-c6cccc7c6ccc6ccccc67)cc5)c5ccc6ccc7ccccc7c6c5)cc4)cccc32)cc1]

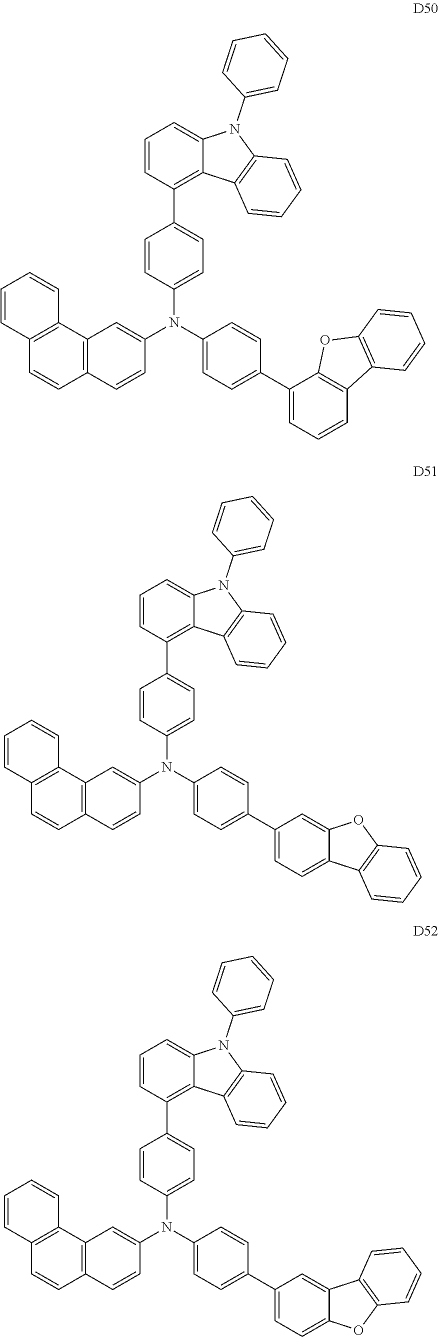 [SMILES: c1ccc(-n2c3ccccc3c3c(-c4ccc(N(c5ccc(-c6cccc7c6oc6ccccc67)cc5)c5ccc6ccc7ccccc7c6c5)cc4)cccc32)cc1 ; c1ccc(-n2c3ccccc3c3c(-c4ccc(N(c5ccc(-c6ccc7c(c6)oc6ccccc67)cc5)c5ccc6ccc7ccccc7c6c5)cc4)cccc32)cc1 ; c1ccc(-n2c3ccccc3c3c(-c4ccc(N(c5ccc(-c6ccc7oc8ccccc8c7c6)cc5)c5ccc6ccc7ccccc7c6c5)cc4)cccc32)cc1]

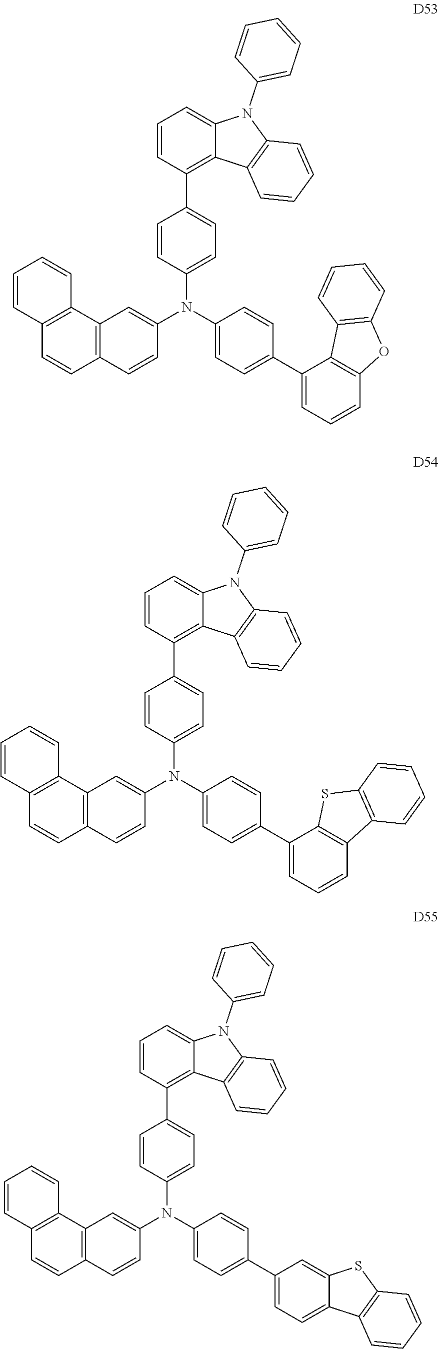 [SMILES: c1ccc(-n2c3ccccc3c3c(-c4ccc(N(c5ccc(-c6cccc7oc8ccccc8c67)cc5)c5ccc6ccc7ccccc7c6c5)cc4)cccc32)cc1 ; c1ccc(-n2c3ccccc3c3c(-c4ccc(N(c5ccc(-c6cccc7c6sc6ccccc67)cc5)c5ccc6ccc7ccccc7c6c5)cc4)cccc32)cc1 ; c1ccc(-n2c3ccccc3c3c(-c4ccc(N(c5ccc(-c6ccc7c(c6)sc6ccccc67)cc5)c5ccc6ccc7ccccc7c6c5)cc4)cccc32)cc1]

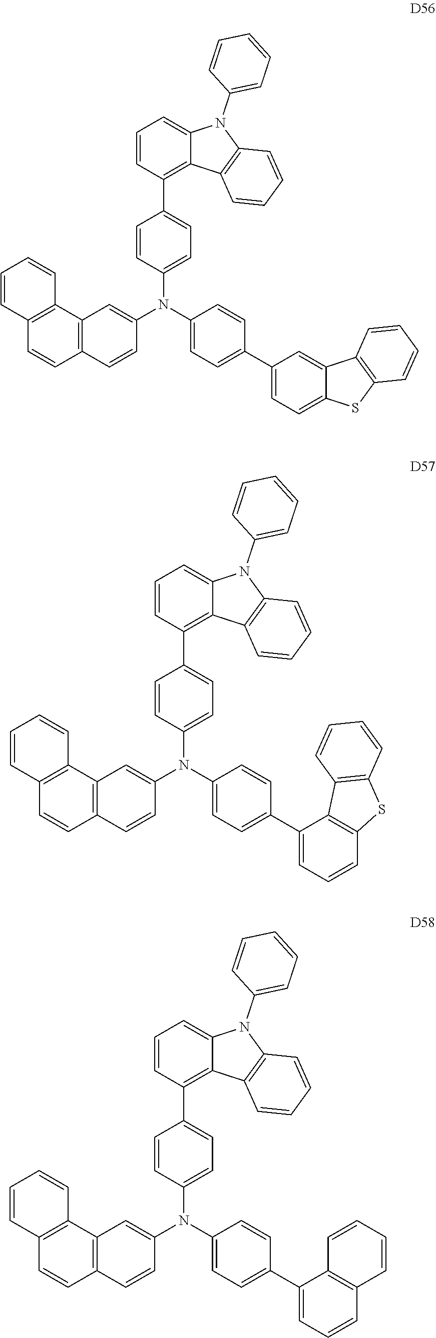 [SMILES: c1ccc(-n2c3ccccc3c3c(-c4ccc(N(c5ccc(-c6ccc7sc8ccccc8c7c6)cc5)c5ccc6ccc7ccccc7c6c5)cc4)cccc32)cc1 ; c1ccc(-n2c3ccccc3c3c(-c4ccc(N(c5ccc(-c6cccc7sc8ccccc8c67)cc5)c5ccc6ccc7ccccc7c6c5)cc4)cccc32)cc1 ; c1ccc(-n2c3ccccc3c3c(-c4ccc(N(c5ccc(-c6cccc7ccccc67)cc5)c5ccc6ccc7ccccc7c6c5)cc4)cccc32)cc1]

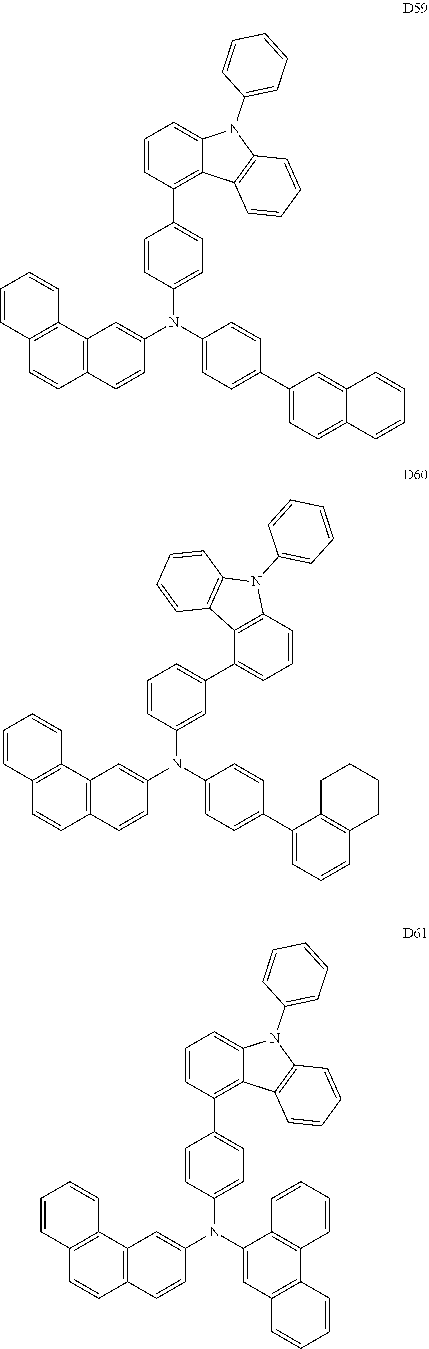 [SMILES: c1ccc(-n2c3ccccc3c3c(-c4ccc(N(c5ccc(-c6ccc7ccccc7c6)cc5)c5ccc6ccc7ccccc7c6c5)cc4)cccc32)cc1 ; c1ccc(-n2c3ccccc3c3c(-c4cccc(N(c5ccc(-c6cccc7c6CCCC7)cc5)c5ccc6ccc7ccccc7c6c5)c4)cccc32)cc1 ; c1ccc(-n2c3ccccc3c3c(-c4ccc(N(c5ccc6ccc7ccccc7c6c5)c5cc6ccccc6c6ccccc56)cc4)cccc32)cc1]

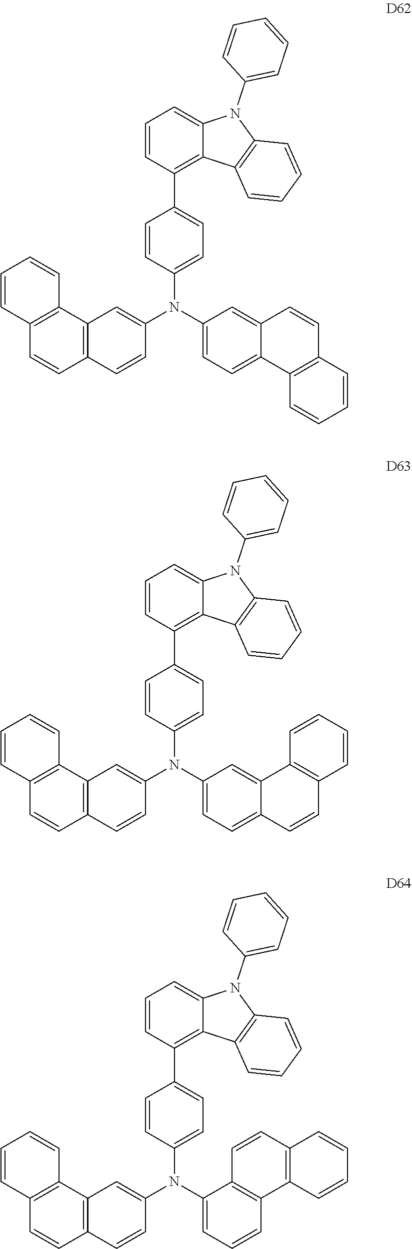 [SMILES: c1ccc(-n2c3ccccc3c3c(-c4ccc(N(c5ccc6c(ccc7ccccc76)c5)c5ccc6ccc7ccccc7c6c5)cc4)cccc32)cc1 ; c1ccc(-n2c3ccccc3c3c(-c4ccc(N(c5ccc6ccc7ccccc7c6c5)c5ccc6ccc7ccccc7c6c5)cc4)cccc32)cc1 ; c1ccc(-n2c3ccccc3c3c(-c4ccc(N(c5ccc6ccc7ccccc7c6c5)c5cccc6c5ccc5ccccc56)cc4)cccc32)cc1]

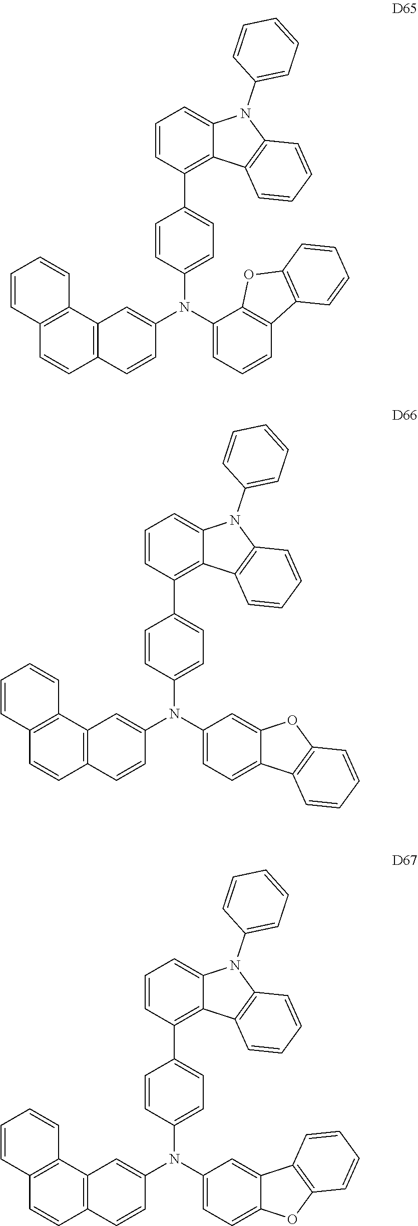 [SMILES: c1ccc(-n2c3ccccc3c3c(-c4ccc(N(c5ccc6ccc7ccccc7c6c5)c5cccc6c5oc5ccccc56)cc4)cccc32)cc1 ; c1ccc(-n2c3ccccc3c3c(-c4ccc(N(c5ccc6c(c5)oc5ccccc56)c5ccc6ccc7ccccc7c6c5)cc4)cccc32)cc1 ; c1ccc(-n2c3ccccc3c3c(-c4ccc(N(c5ccc6ccc7ccccc7c6c5)c5ccc6oc7ccccc7c6c5)cc4)cccc32)cc1]

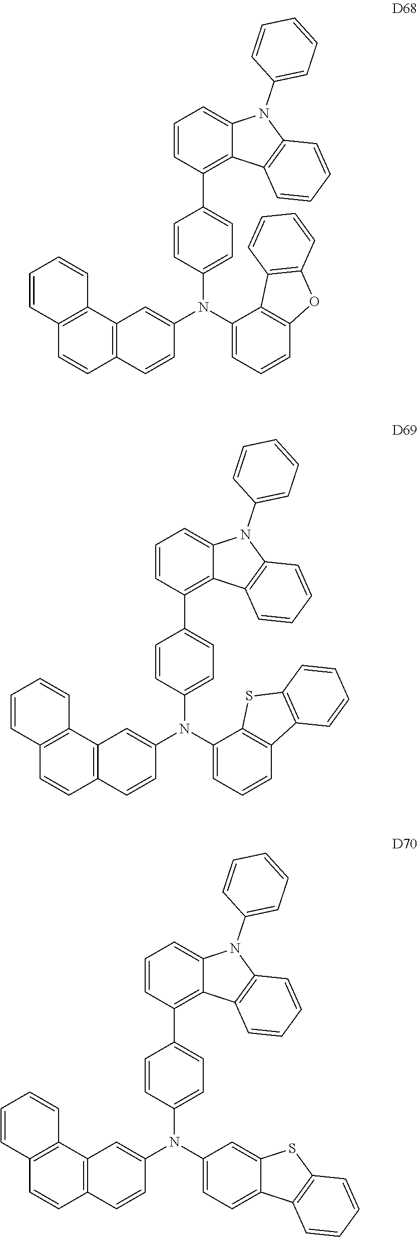 [SMILES: c1ccc(-n2c3ccccc3c3c(-c4ccc(N(c5ccc6ccc7ccccc7c6c5)c5cccc6oc7ccccc7c56)cc4)cccc32)cc1 ; c1ccc(-n2c3ccccc3c3c(-c4ccc(N(c5ccc6ccc7ccccc7c6c5)c5cccc6c5sc5ccccc56)cc4)cccc32)cc1 ; c1ccc(-n2c3ccccc3c3c(-c4ccc(N(c5ccc6c(c5)sc5ccccc56)c5ccc6ccc7ccccc7c6c5)cc4)cccc32)cc1]

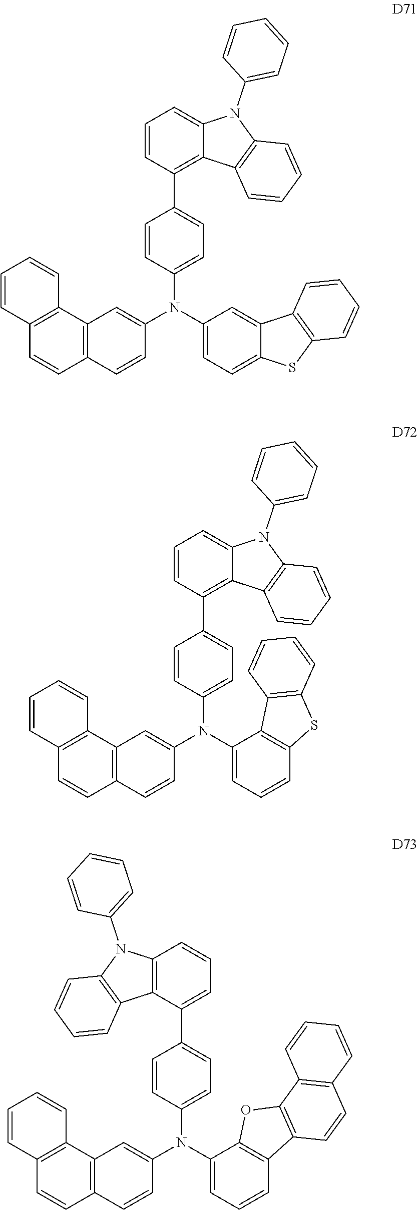 [SMILES: c1ccc(-n2c3ccccc3c3c(-c4ccc(N(c5ccc6ccc7ccccc7c6c5)c5ccc6sc7ccccc7c6c5)cc4)cccc32)cc1 ; c1ccc(-n2c3ccccc3c3c(-c4ccc(N(c5ccc6ccc7ccccc7c6c5)c5cccc6sc7ccccc7c56)cc4)cccc32)cc1 ; c1ccc(-n2c3ccccc3c3c(-c4ccc(N(c5ccc6ccc7ccccc7c6c5)c5cccc6c5oc5c7ccccc7ccc65)cc4)cccc32)cc1]

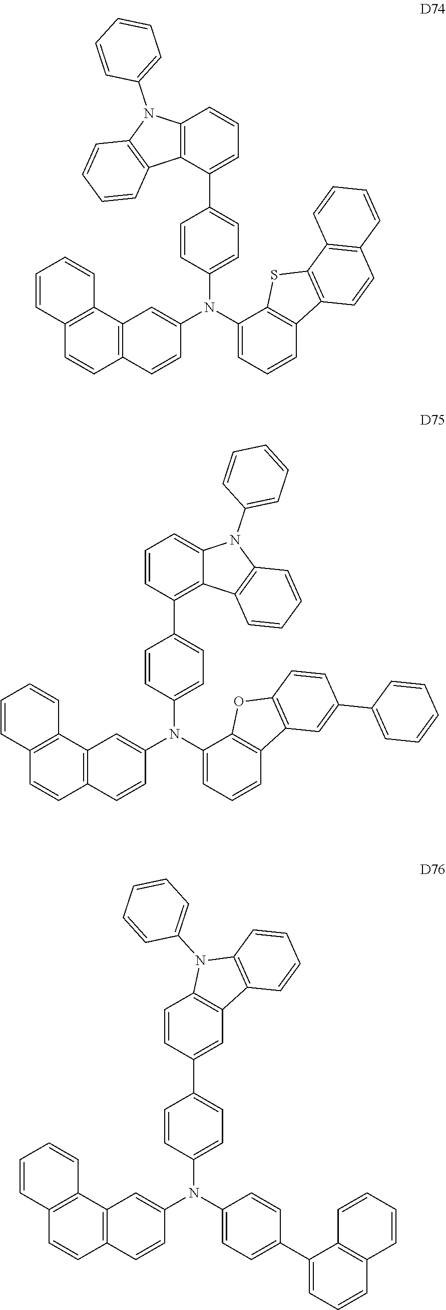 [SMILES: c1ccc(-n2c3ccccc3c3c(-c4ccc(N(c5ccc6ccc7ccccc7c6c5)c5cccc6c5sc5c7ccccc7ccc65)cc4)cccc32)cc1 ; c1ccc(-c2ccc3oc4c(N(c5ccc(-c6cccc7c6c6ccccc6n7-c6ccccc6)cc5)c5ccc6ccc7ccccc7c6c5)cccc4c3c2)cc1 ; c1ccc(-n2c3ccccc3c3cc(-c4ccc(N(c5ccc(-c6cccc7ccccc67)cc5)c5ccc6ccc7ccccc7c6c5)cc4)ccc32)cc1]

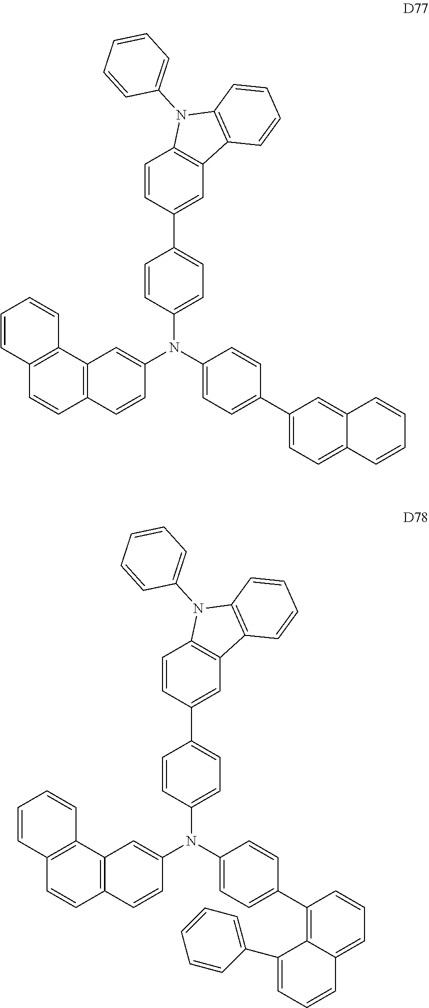 [SMILES: c1ccc(-n2c3ccccc3c3cc(-c4ccc(N(c5ccc(-c6ccc7ccccc7c6)cc5)c5ccc6ccc7ccccc7c6c5)cc4)ccc32)cc1 ; c1ccc(-c2cccc3cccc(-c4ccc(N(c5ccc(-c6ccc7c(c6)c6ccccc6n7-c6ccccc6)cc5)c5ccc6ccc7ccccc7c6c5)cc4)c23)cc1]

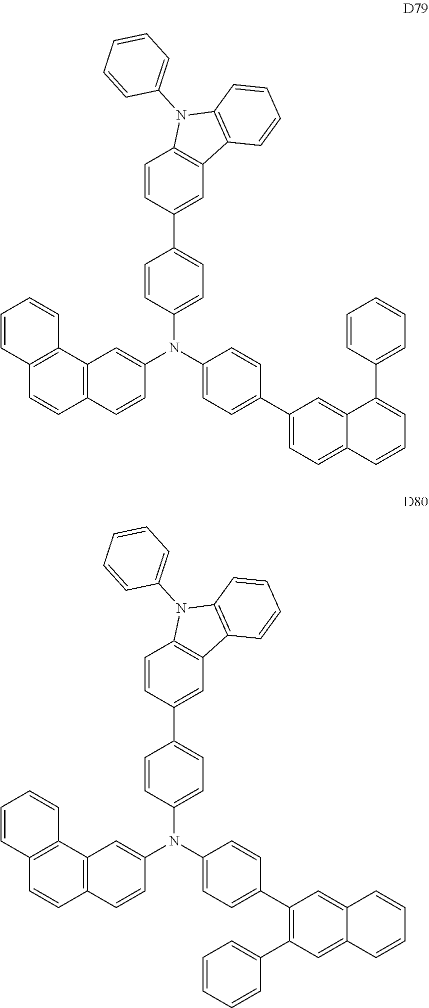 [SMILES: c1ccc(-c2cccc3ccc(-c4ccc(N(c5ccc(-c6ccc7c(c6)c6ccccc6n7-c6ccccc6)cc5)c5ccc6ccc7ccccc7c6c5)cc4)cc23)cc1 ; c1ccc(-c2cc3ccccc3cc2-c2ccc(N(c3ccc(-c4ccc5c(c4)c4ccccc4n5-c4ccccc4)cc3)c3ccc4ccc5ccccc5c4c3)cc2)cc1]

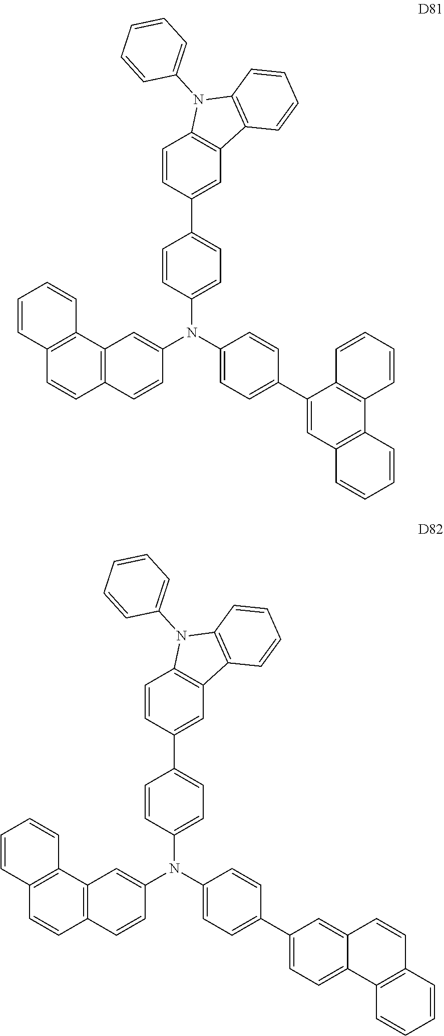 [SMILES: c1ccc(-n2c3ccccc3c3cc(-c4ccc(N(c5ccc(-c6cc7ccccc7c7ccccc67)cc5)c5ccc6ccc7ccccc7c6c5)cc4)ccc32)cc1 ; c1ccc(-n2c3ccccc3c3cc(-c4ccc(N(c5ccc(-c6ccc7c(ccc8ccccc87)c6)cc5)c5ccc6ccc7ccccc7c6c5)cc4)ccc32)cc1]

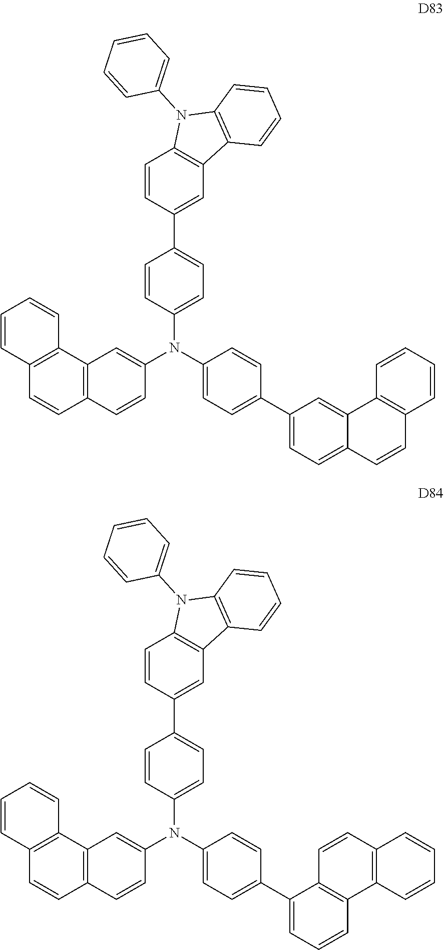 [SMILES: c1ccc(-n2c3ccccc3c3cc(-c4ccc(N(c5ccc(-c6ccc7ccc8ccccc8c7c6)cc5)c5ccc6ccc7ccccc7c6c5)cc4)ccc32)cc1 ; c1ccc(-n2c3ccccc3c3cc(-c4ccc(N(c5ccc(-c6cccc7c6ccc6ccccc67)cc5)c5ccc6ccc7ccccc7c6c5)cc4)ccc32)cc1]

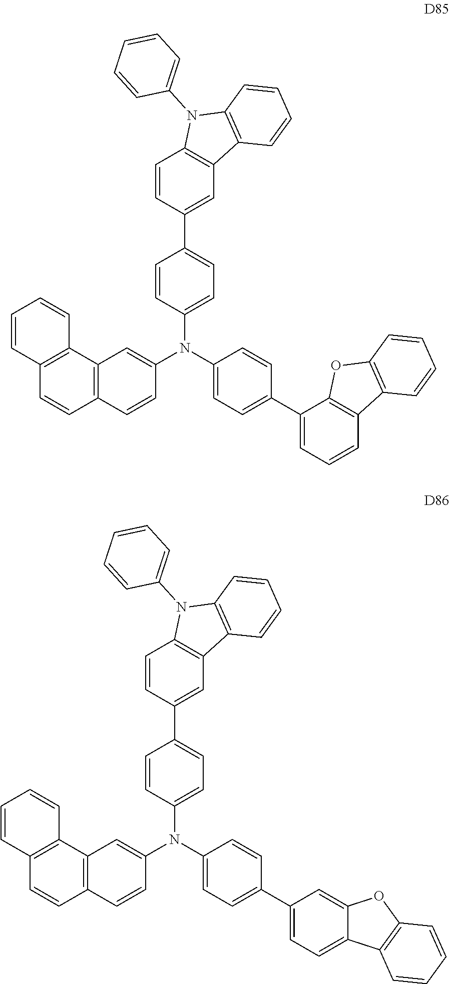 [SMILES: c1ccc(-n2c3ccccc3c3cc(-c4ccc(N(c5ccc(-c6cccc7c6oc6ccccc67)cc5)c5ccc6ccc7ccccc7c6c5)cc4)ccc32)cc1 ; c1ccc(-n2c3ccccc3c3cc(-c4ccc(N(c5ccc(-c6ccc7c(c6)oc6ccccc67)cc5)c5ccc6ccc7ccccc7c6c5)cc4)ccc32)cc1]

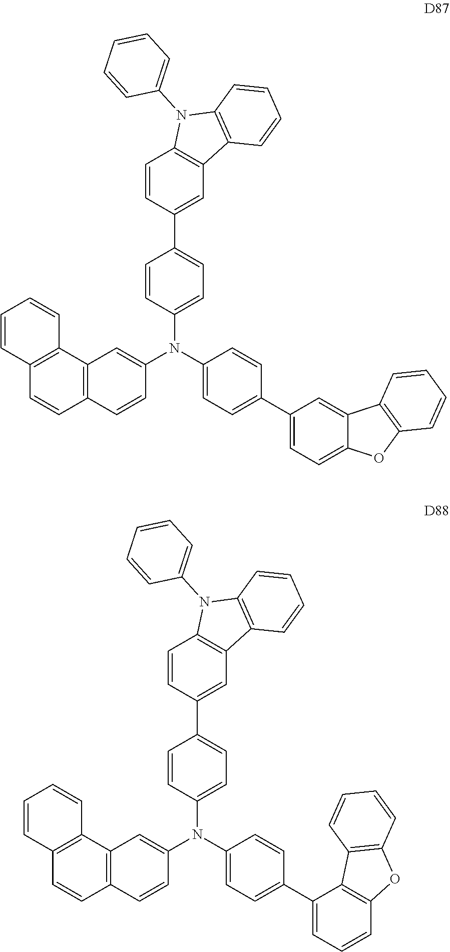 [SMILES: c1ccc(-n2c3ccccc3c3cc(-c4ccc(N(c5ccc(-c6ccc7oc8ccccc8c7c6)cc5)c5ccc6ccc7ccccc7c6c5)cc4)ccc32)cc1 ; c1ccc(-n2c3ccccc3c3cc(-c4ccc(N(c5ccc(-c6cccc7oc8ccccc8c67)cc5)c5ccc6ccc7ccccc7c6c5)cc4)ccc32)cc1]

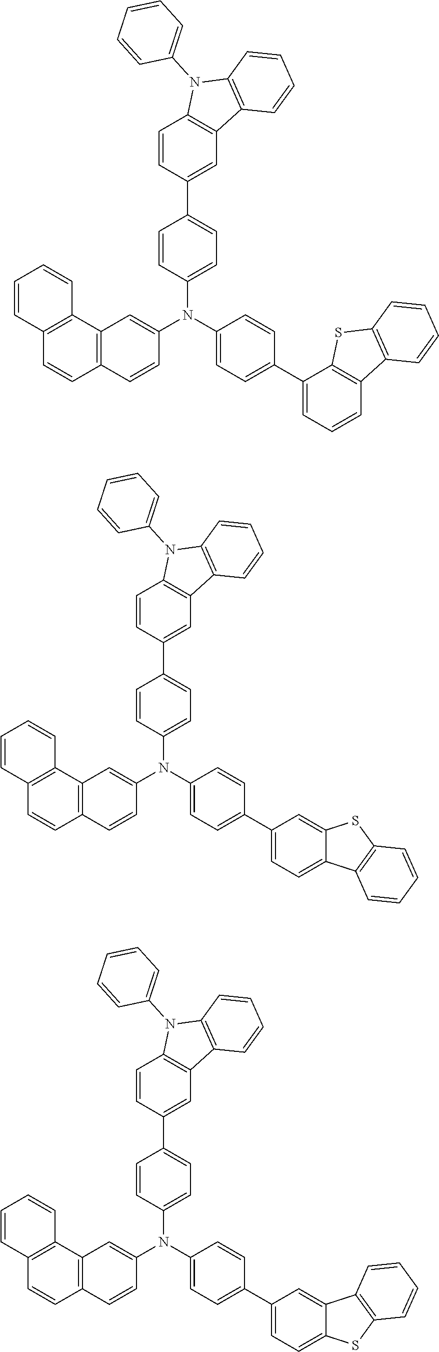 [SMILES: c1ccc(-n2c3ccccc3c3cc(-c4ccc(N(c5ccc(-c6cccc7c6sc6ccccc67)cc5)c5ccc6ccc7ccccc7c6c5)cc4)ccc32)cc1 ; c1ccc(-n2c3ccccc3c3cc(-c4ccc(N(c5ccc(-c6ccc7c(c6)sc6ccccc67)cc5)c5ccc6ccc7ccccc7c6c5)cc4)ccc32)cc1 ; c1ccc(-n2c3ccccc3c3cc(-c4ccc(N(c5ccc(-c6ccc7sc8ccccc8c7c6)cc5)c5ccc6ccc7ccccc7c6c5)cc4)ccc32)cc1]

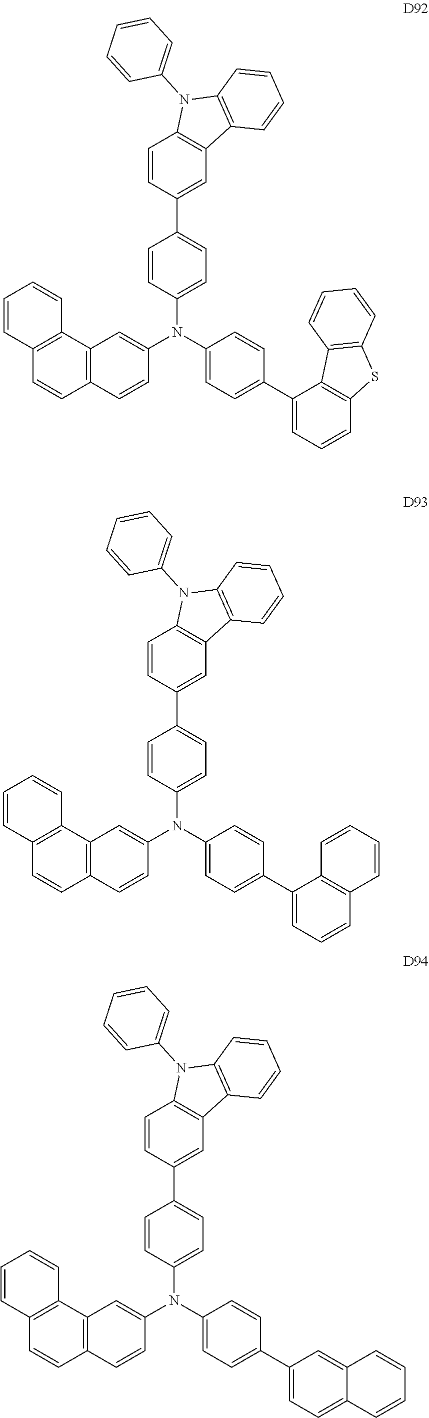 [SMILES: c1ccc(-n2c3ccccc3c3cc(-c4ccc(N(c5ccc(-c6cccc7sc8ccccc8c67)cc5)c5ccc6ccc7ccccc7c6c5)cc4)ccc32)cc1 ; c1ccc(-n2c3ccccc3c3cc(-c4ccc(N(c5ccc(-c6cccc7ccccc67)cc5)c5ccc6ccc7ccccc7c6c5)cc4)ccc32)cc1 ; c1ccc(-n2c3ccccc3c3cc(-c4ccc(N(c5ccc(-c6ccc7ccccc7c6)cc5)c5ccc6ccc7ccccc7c6c5)cc4)ccc32)cc1]

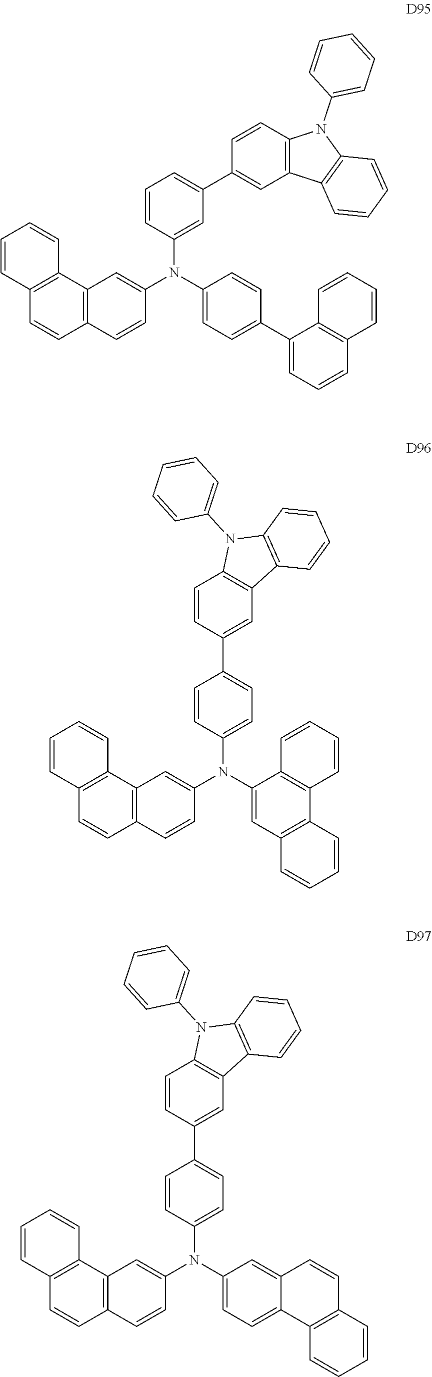 [SMILES: c1ccc(-n2c3ccccc3c3cc(-c4cccc(N(c5ccc(-c6cccc7ccccc67)cc5)c5ccc6ccc7ccccc7c6c5)c4)ccc32)cc1 ; c1ccc(-n2c3ccccc3c3cc(-c4ccc(N(c5ccc6ccc7ccccc7c6c5)c5cc6ccccc6c6ccccc56)cc4)ccc32)cc1 ; c1ccc(-n2c3ccccc3c3cc(-c4ccc(N(c5ccc6c(ccc7ccccc76)c5)c5ccc6ccc7ccccc7c6c5)cc4)ccc32)cc1]

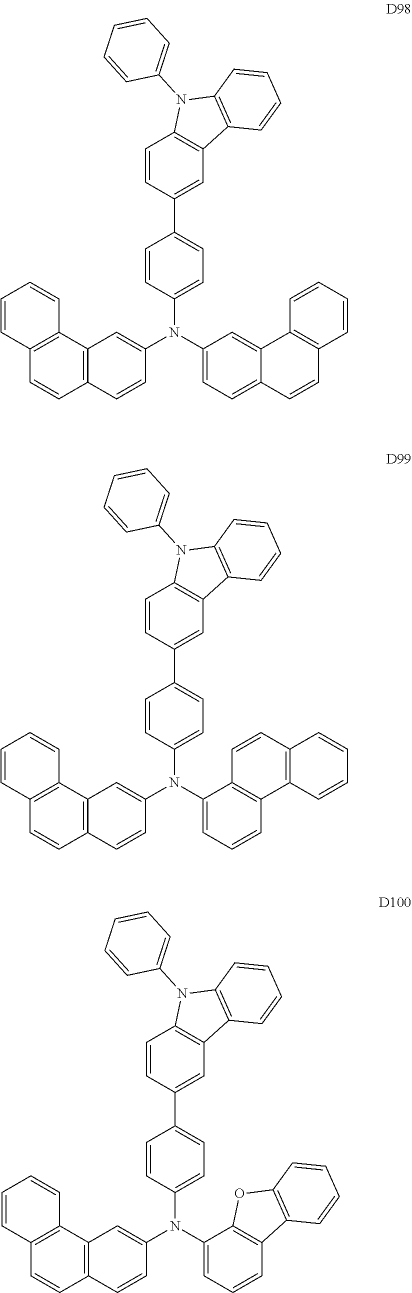 [SMILES: c1ccc(-n2c3ccccc3c3cc(-c4ccc(N(c5ccc6ccc7ccccc7c6c5)c5ccc6ccc7ccccc7c6c5)cc4)ccc32)cc1 ; c1ccc(-n2c3ccccc3c3cc(-c4ccc(N(c5ccc6ccc7ccccc7c6c5)c5cccc6c5ccc5ccccc56)cc4)ccc32)cc1 ; c1ccc(-n2c3ccccc3c3cc(-c4ccc(N(c5ccc6ccc7ccccc7c6c5)c5cccc6c5oc5ccccc56)cc4)ccc32)cc1]

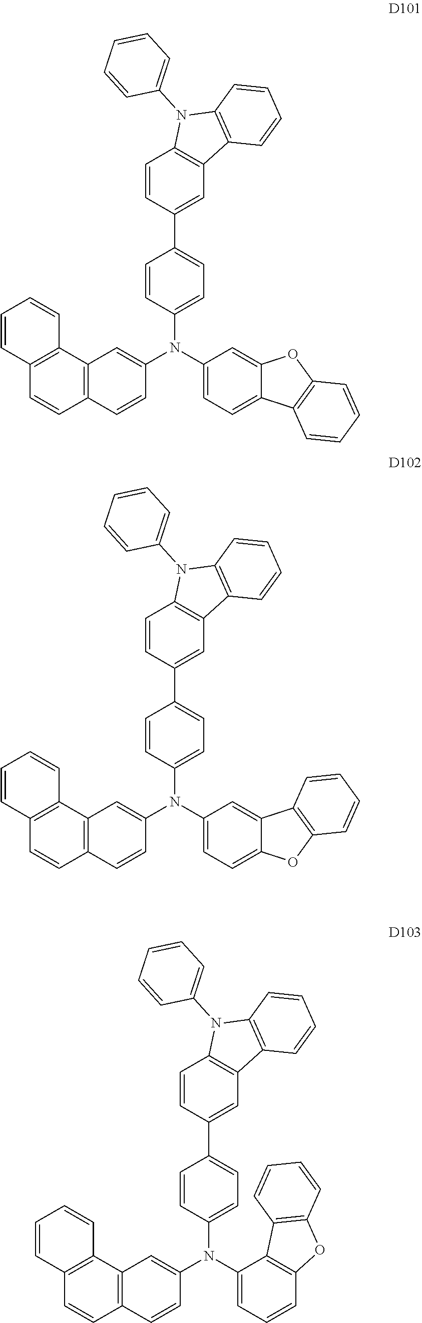 [SMILES: c1ccc(-n2c3ccccc3c3cc(-c4ccc(N(c5ccc6c(c5)oc5ccccc56)c5ccc6ccc7ccccc7c6c5)cc4)ccc32)cc1 ; c1ccc(-n2c3ccccc3c3cc(-c4ccc(N(c5ccc6ccc7ccccc7c6c5)c5ccc6oc7ccccc7c6c5)cc4)ccc32)cc1 ; c1ccc(-n2c3ccccc3c3cc(-c4ccc(N(c5ccc6ccc7ccccc7c6c5)c5cccc6oc7ccccc7c56)cc4)ccc32)cc1]

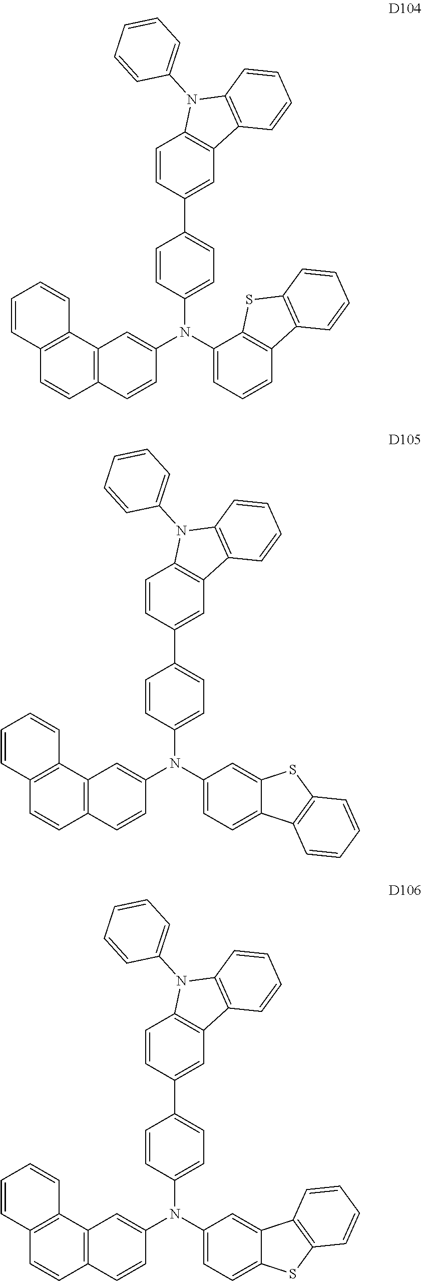 [SMILES: c1ccc(-n2c3ccccc3c3cc(-c4ccc(N(c5ccc6ccc7ccccc7c6c5)c5cccc6c5sc5ccccc56)cc4)ccc32)cc1 ; c1ccc(-n2c3ccccc3c3cc(-c4ccc(N(c5ccc6c(c5)sc5ccccc56)c5ccc6ccc7ccccc7c6c5)cc4)ccc32)cc1 ; c1ccc(-n2c3ccccc3c3cc(-c4ccc(N(c5ccc6ccc7ccccc7c6c5)c5ccc6sc7ccccc7c6c5)cc4)ccc32)cc1]

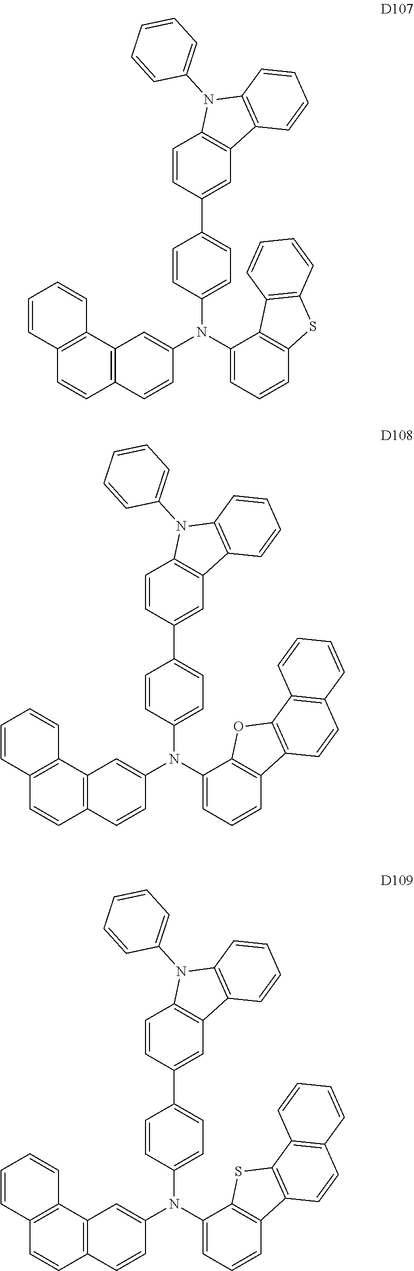

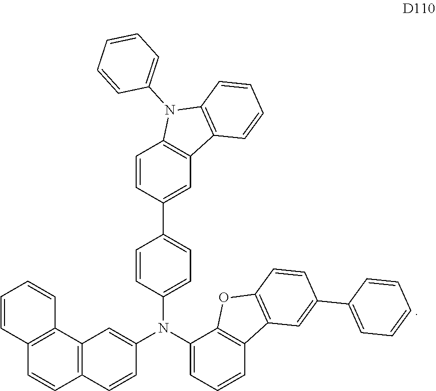 [SMILES: c1ccc(-c2ccc3oc4c(N(c5ccc(-c6ccc7c(c6)c6ccccc6n7-c6ccccc6)cc5)c5ccc6ccc7ccccc7c6c5)cccc4c3c2)cc1]

15. An amine compound represented by Formula 1 below: 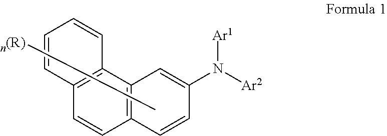
wherein, in Formula 1 above,
R is a hydrogen atom, a deuterium atom, a halogen atom, or a substituted or unsubstituted alkyl group having 1 to 20 carbon atoms,
n is an integer of 0 to 9, and
Ar¹ and Ar² are each independently represented by any one selected from among Formula 2 to Formula 5 below: 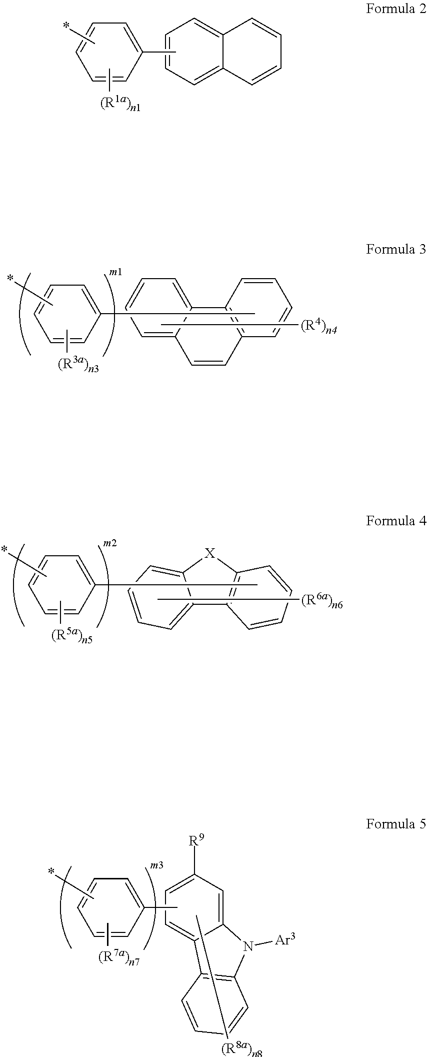
wherein, in Formula 4 above, X is O or S,
in Formula 5 above, Ar³ is a substituted or unsubstituted phenyl group, and
in Formula 2 to Formula 5 above,
R¹ to R⁵, R⁷, and R⁹ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms,
R⁶ and R⁸ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, or adjacent R⁶'s or adjacent R⁸'s are bonded to each to form an aromatic ring,
n1, n3, n5, and n7 are each independently an integer of 0 to 4, n2 and n6 are each independently an integer of 0 to 7, n4 is an integer of 0 to 9, and n8 is an integer of 0 to 6,
m1 to m3 are each independently 0 or 1,
when Ar¹ and Ar² are each independently represented by any one selected from among Formula 3 to Formula 5 above, at least one selected from among m1 to m3 is 1,
the case where both Ar¹ and Ar² are represented by Formula 3 above is excluded,
when both Ar¹ and Ar² are represented by Formula 4 above, any one selected from among two m2's is 1, and the other is 0, and
a structure in which any hydrogen atom in the molecule is substituted with a deuterium atom is included.

16. The amine compound of claim 15, wherein Formula 2 above is represented by Formula 2-1 or Formula 2-2 below: 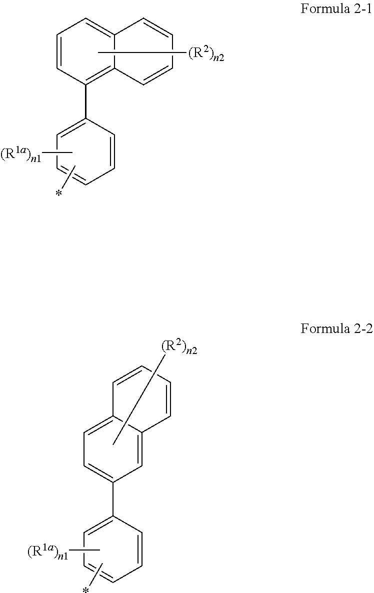
wherein, in Formula 2-1 and Formula 2-2 above,
R^{1a} is a hydrogen atom, a deuterium atom, or a substituted or unsubstituted phenyl group, and
n1, n2, and R² are the same as defined with respect to Formula 2 above.

17. The amine compound of claim 16, wherein Formula 2-1 above is represented by 2-a or 2-b below, and Formula 2-2 above is represented by 2-c or 2-d below: 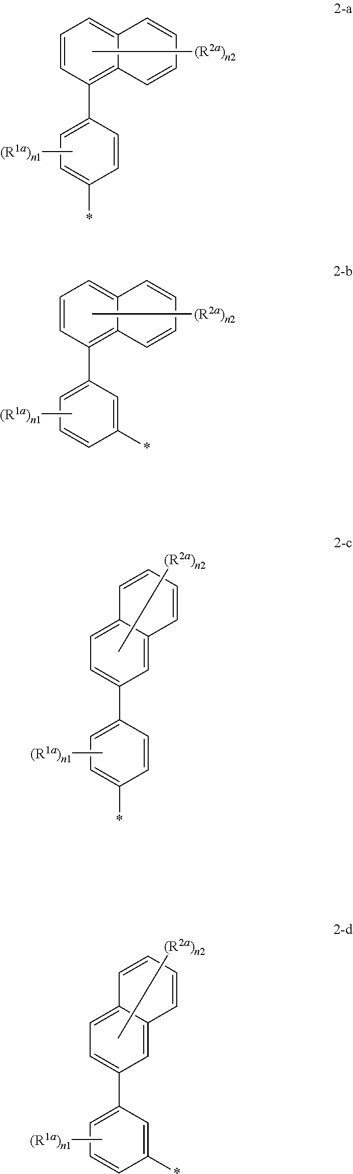
wherein, in 2-a to 2-d above,
R^{2a} is a hydrogen atom, a deuterium atom, or a substituted or unsubstituted aryl group having 6 to 15 carbon atoms, and
n1, n2, and R^{1a} are the same as defined with respect to Formula 2-1 and Formula 2-2 above.

18. The amine compound of claim 15, wherein Formula 3 above is represented by any one selected from among Formula 3-1 to Formula 3-5 below: 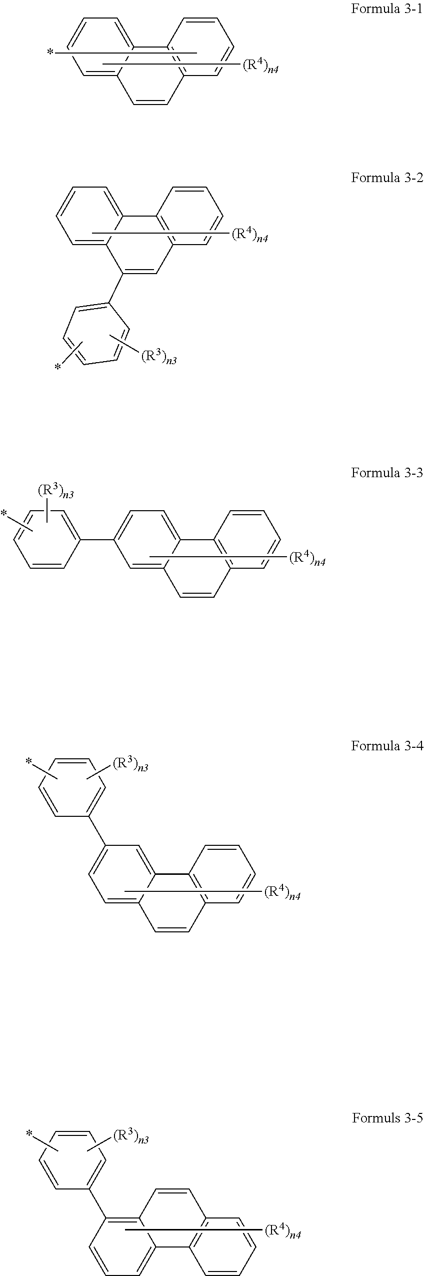
wherein, in Formula 3-1 to Formula 3-5 above,
R^{3a} is a hydrogen atom, a deuterium atom, or a substituted or unsubstituted phenyl group, and
n3, n4, and R⁴ are the same as defined with respect to Formula 3 above.

19. The amine compound of claim 18, wherein Formula 3-1 above is represented by any one selected from among 3-a to 3-d below,
Formula 3-2 above is represented by 3-e below,
Formula 3-3 above is represented by 3-f below,
Formula 3-4 above is represented by 3-g below, and
Formula 3-5 above is represented by 3-h below: 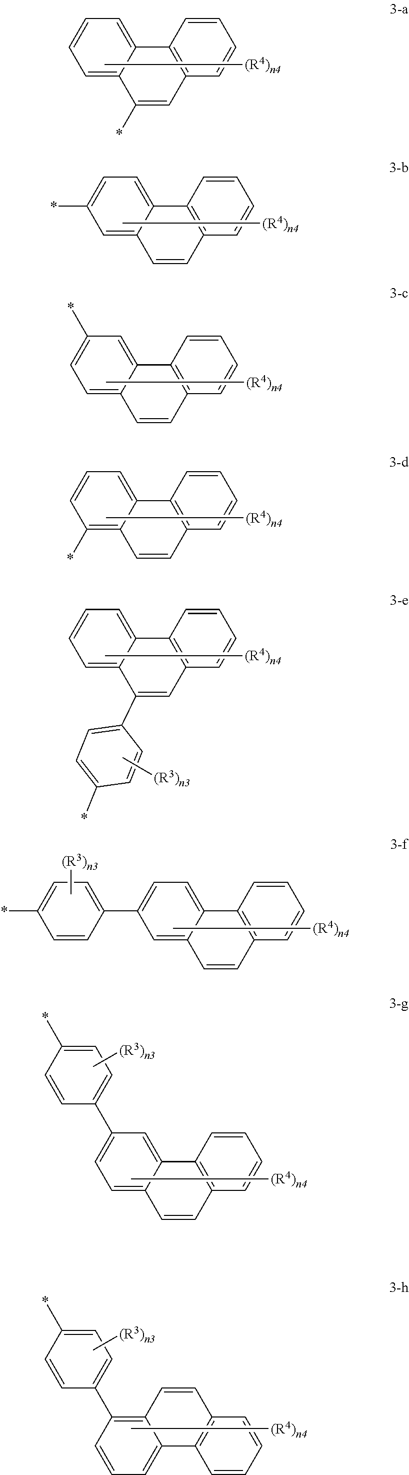
wherein, in 3-a to 3-h above,
n3, n4, and R^{3a} are the same as defined with respect to Formula 3-1 to Formula 3-5 above, and
R^{4a} is a hydrogen atom, a deuterium atom, a halogen atom, or a substituted or unsubstituted alkyl group having 1 to 10 carbon atoms.

20. The amine compound of claim 15, wherein Formula 4 above is represented by Formula 4-1 or Formula 4-2 below: 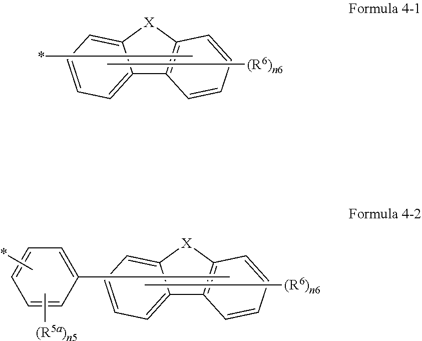
wherein, in Formula 4-1 and Formula 4-2 above,
R^{5a} is a hydrogen atom, a deuterium atom, or a substituted or unsubstituted phenyl group, and
X, n5, n6, and R⁶ are the same as defined with respect to Formula 4 above.

21. The amine compound of claim 20, wherein Formula 4-1 above is represented by 4-a below, and Formula 4-2 above is represented by 4-b or 4-c below: 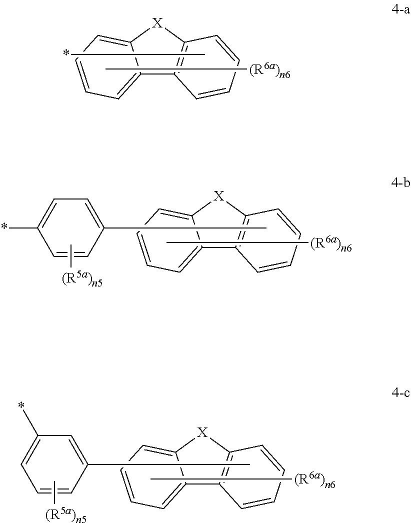
wherein, in 4-a to 4-c above,
R^{6a} is a hydrogen atom, a deuterium atom, or a substituted or unsubstituted aryl group having 6 to 15 ring-forming carbon atoms, or adjacent R^{6a}'s are bonded to each other to form an aromatic ring, and
X, n5, n6 and R^{5a} are the same as defined with respect to Formula 4-1 and Formula 4-2 above.

22. The amine compound of claim 15, wherein Formula 5 above is represented by any one selected from among Formula 5-1 to Formula 5-4 below: 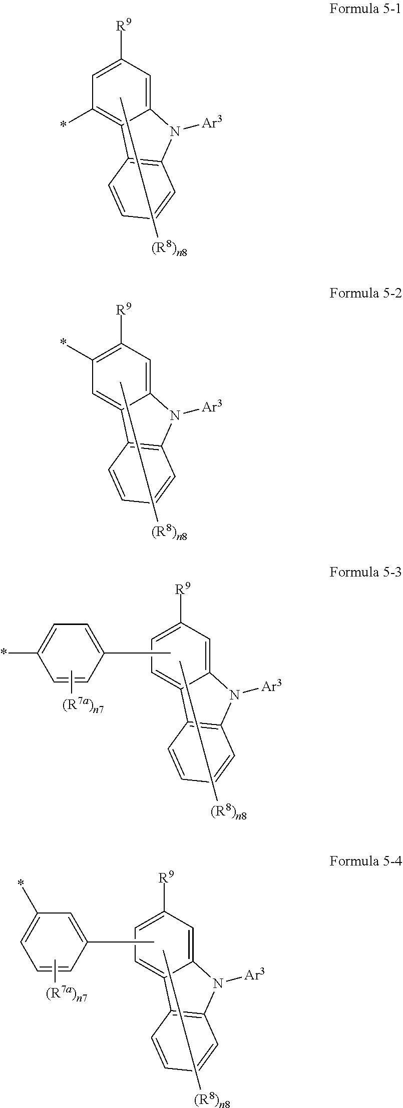
wherein, in Formula 5-1 to Formula 5-4 above,
R^{7a} is a hydrogen atom or a deuterium atom, and
n7, n8, R⁸, R⁹, and Ar₃ are the same as defined with respect to Formula 5 above.

23. The amine compound of claim 22, wherein Formula 5-1 above is represented by 5-a below,
Formula 5-2 above is represented by 5-b below,
Formula 5-3 above is represented by 5-c or 5-d below, and
Formula 5-4 above is represented by 5-e or 5-f below: 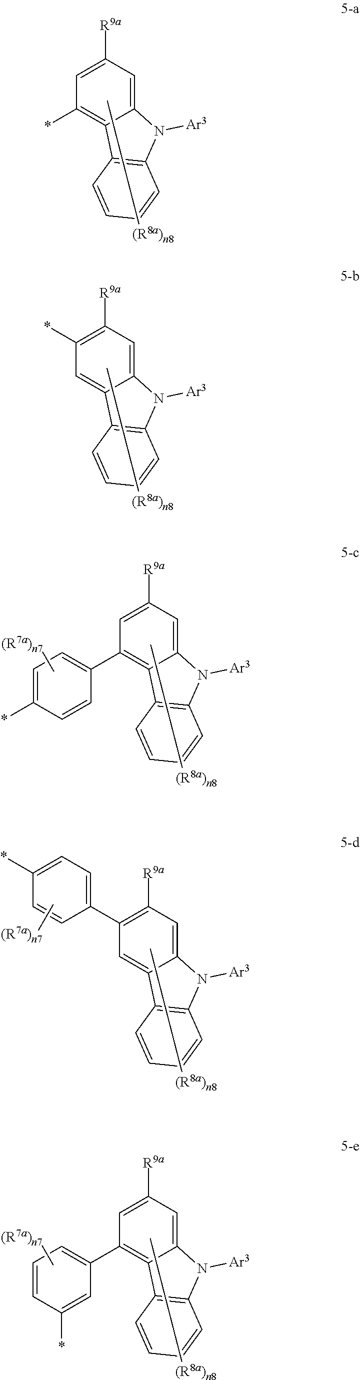 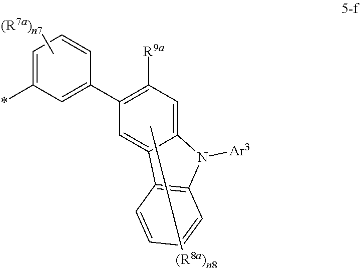
wherein, in 5-a to 5-f above,
R^{8a} is a hydrogen atom, a deuterium atom, a substituted or unsubstituted aryl group having 6 to 15 ring-forming carbon atoms, or adjacent R^{8a}'s are bonded to each other to form an aromatic ring,
R^{9a} is a hydrogen atom or a deuterium atom, and
n7, n8, R^{7a}, and Ar³ are the same as defined with respect to Formula 5-1 to Formula 5-4 above.

24. The amine compound of claim 15, wherein R above is a hydrogen atom, a deuterium atom, a halogen atom, or a substituted or unsubstituted t-butyl group.

25. The amine compound of claim 15, wherein Formula 1 above is represented by any one selected from among compounds of Compound Group 1 below: 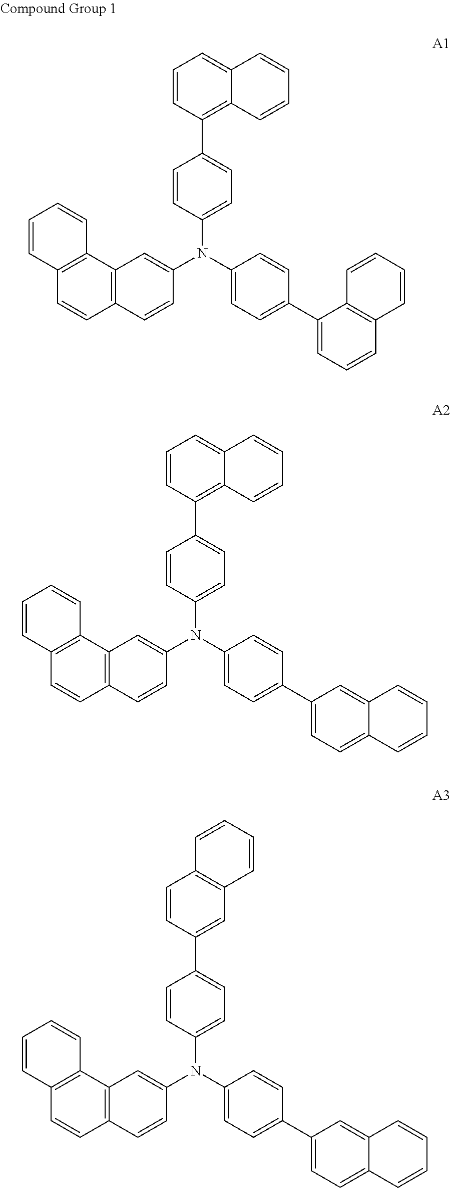 [SMILES: c1ccc2c(-c3ccc(N(c4ccc(-c5cccc6ccccc56)cc4)c4ccc5ccc6ccccc6c5c4)cc3)cccc2c1 ; c1ccc2cc(-c3ccc(N(c4ccc(-c5cccc6ccccc56)cc4)c4ccc5ccc6ccccc6c5c4)cc3)ccc2c1 ; c1ccc2cc(-c3ccc(N(c4ccc(-c5ccc6ccccc6c5)cc4)c4ccc5ccc6ccccc6c5c4)cc3)ccc2c1] 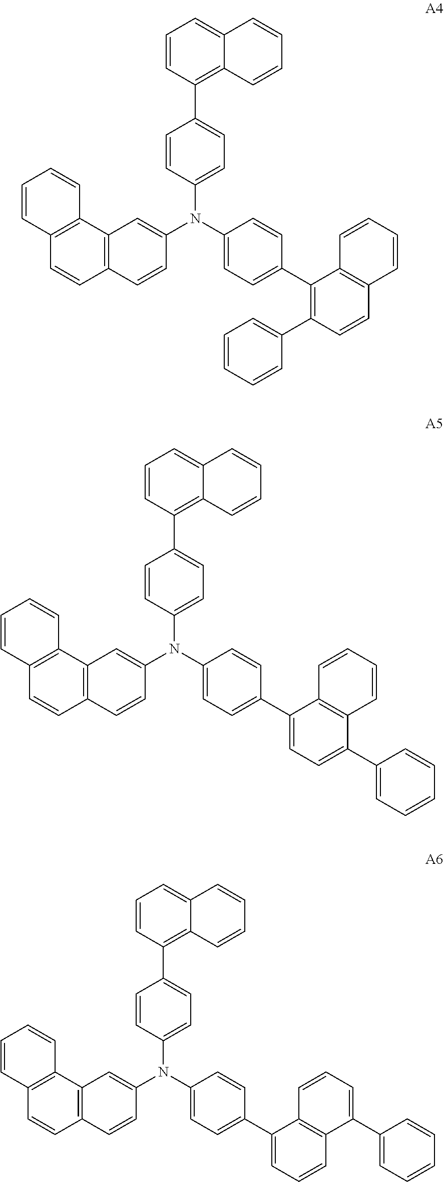 [SMILES: c1ccc(-c2ccc3ccccc3c2-c2ccc(N(c3ccc(-c4cccc5ccccc45)cc3)c3ccc4ccc5ccccc5c4c3)cc2)cc1 ; c1ccc(-c2ccc(-c3ccc(N(c4ccc(-c5cccc6ccccc56)cc4)c4ccc5ccc6ccccc6c5c4)cc3)c3ccccc23)cc1 ; c1ccc(-c2cccc3c(-c4ccc(N(c5ccc(-c6cccc7ccccc67)cc5)c5ccc6ccc7ccccc7c6c5)cc4)cccc23)cc1] 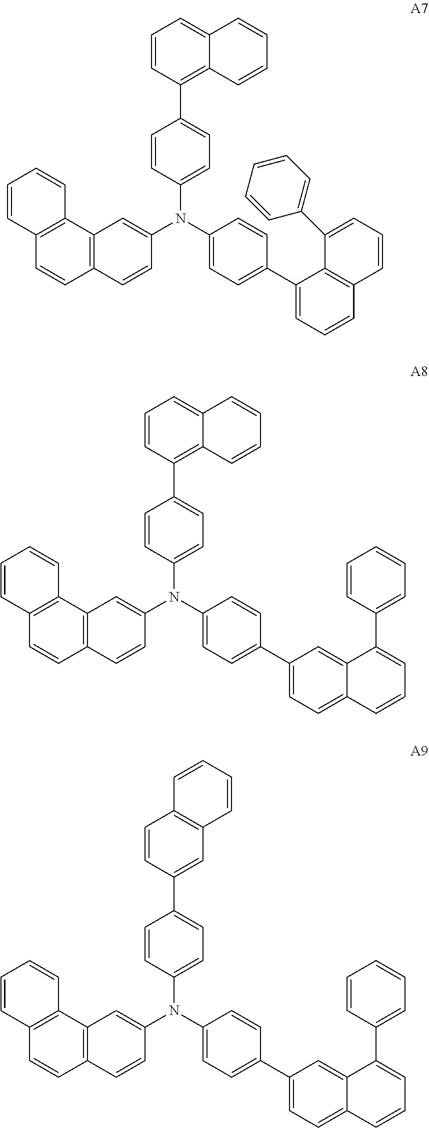 [SMILES: c1ccc(-c2cccc3cccc(-c4ccc(N(c5ccc(-c6cccc7ccccc67)cc5)c5ccc6ccc7ccccc7c6c5)cc4)c23)cc1 ; c1ccc(-c2cccc3ccc(-c4ccc(N(c5ccc(-c6cccc7ccccc67)cc5)c5ccc6ccc7ccccc7c6c5)cc4)cc23)cc1 ; c1ccc(-c2cccc3ccc(-c4ccc(N(c5ccc(-c6ccc7ccccc7c6)cc5)c5ccc6ccc7ccccc7c6c5)cc4)cc23)cc1] 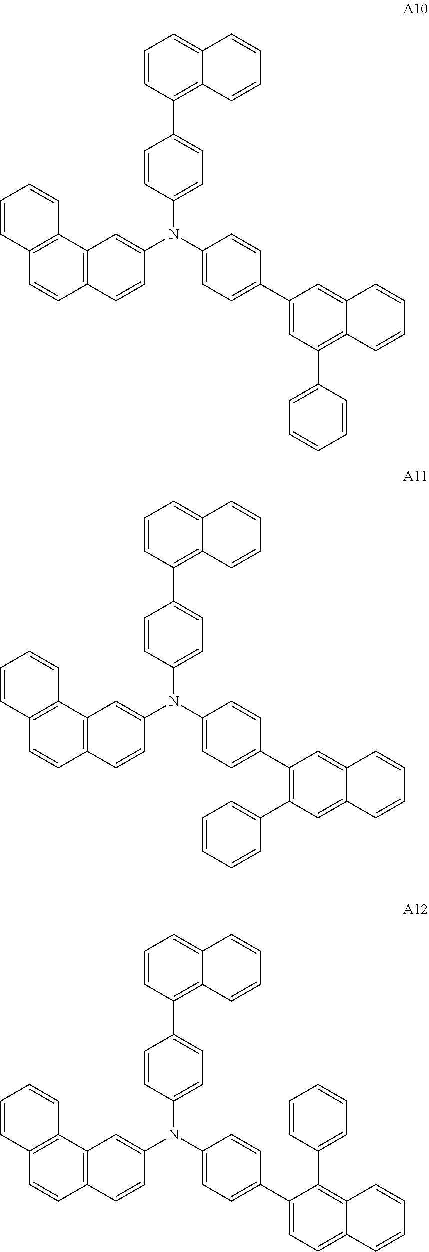 [SMILES: c1ccc(-c2cc(-c3ccc(N(c4ccc(-c5cccc6ccccc56)cc4)c4ccc5ccc6ccccc6c5c4)cc3)cc3ccccc23)cc1 ; c1ccc(-c2cc3ccccc3cc2-c2ccc(N(c3ccc(-c4cccc5ccccc45)cc3)c3ccc4ccc5ccccc5c4c3)cc2)cc1 ; c1ccc(-c2c(-c3ccc(N(c4ccc(-c5cccc6ccccc56)cc4)c4ccc5ccc6ccccc6c5c4)cc3)ccc3ccccc23)cc1] 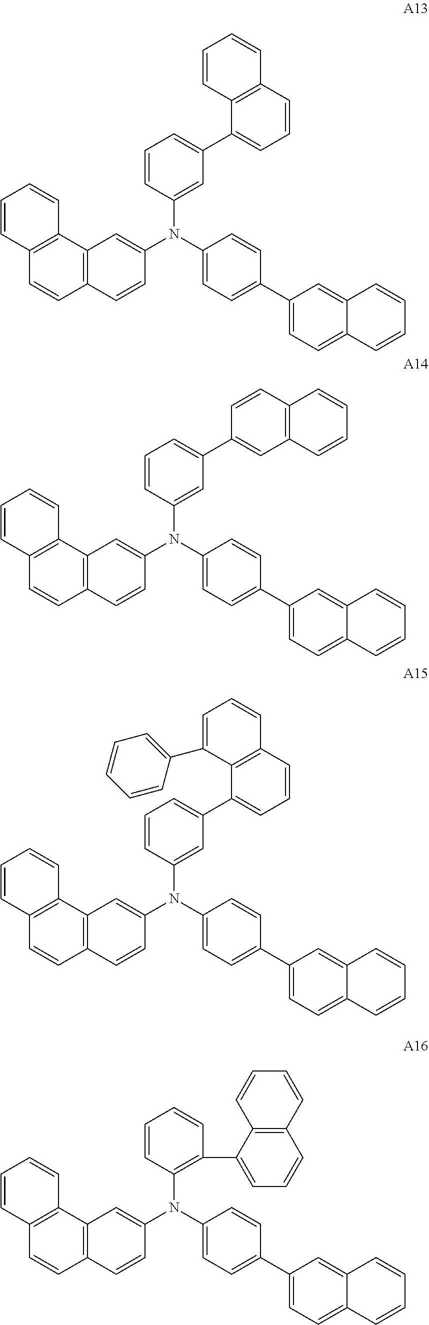 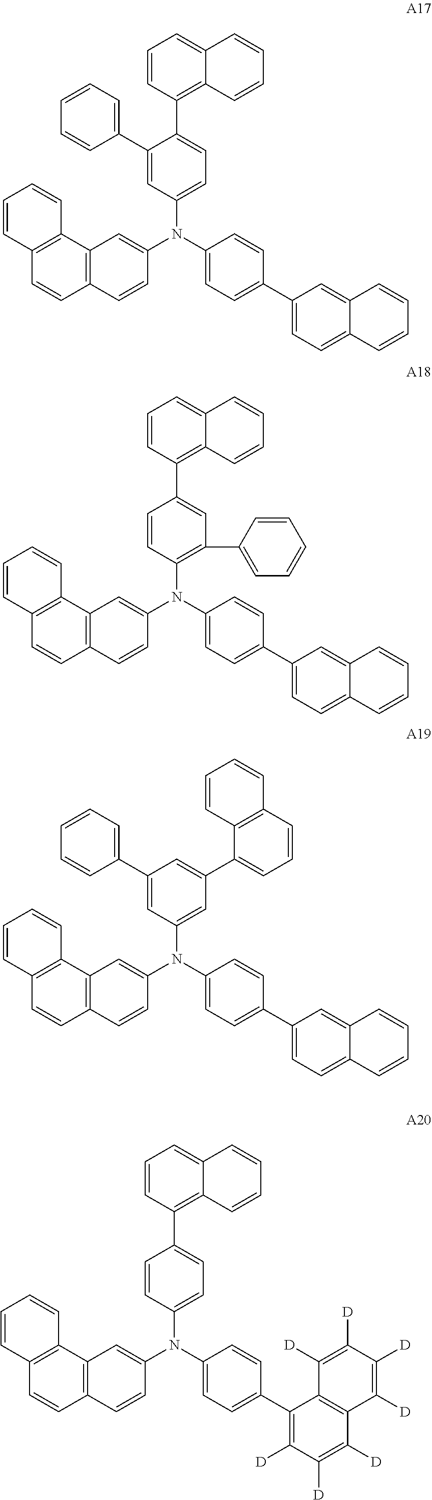 [SMILES: c1ccc(-c2cc(N(c3ccc(-c4ccc5ccccc5c4)cc3)c3ccc4ccc5ccccc5c4c3)ccc2-c2cccc3ccccc23)cc1 ; c1ccc(-c2cc(-c3cccc4ccccc34)ccc2N(c2ccc(-c3ccc4ccccc4c3)cc2)c2ccc3ccc4ccccc4c3c2)cc1 ; c1ccc(-c2cc(-c3cccc4ccccc34)cc(N(c3ccc(-c4ccc5ccccc5c4)cc3)c3ccc4ccc5ccccc5c4c3)c2)cc1 ; [2H]c1c([2H])c([2H])c2c(-c3ccc(N(c4ccc(-c5cccc6ccccc56)cc4)c4ccc5ccc6ccccc6c5c4)cc3)c([2H])c([2H])c([2H])c2c1[2H]] 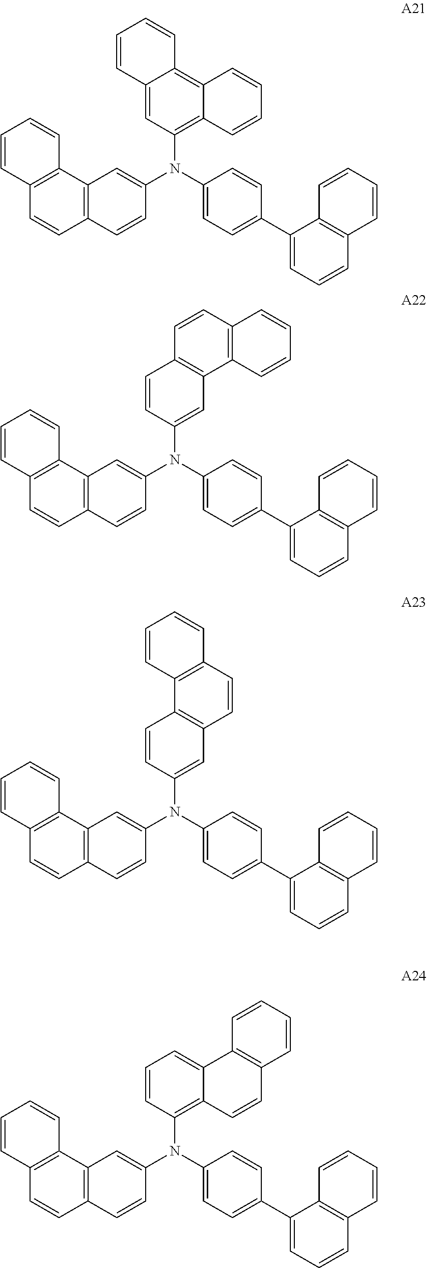 [SMILES: c1ccc2c(-c3ccc(N(c4ccc5ccc6ccccc6c5c4)c4cc5ccccc5c5ccccc45)cc3)cccc2c1 ; c1ccc2c(-c3ccc(N(c4ccc5ccc6ccccc6c5c4)c4ccc5ccc6ccccc6c5c4)cc3)cccc2c1 ; c1ccc2c(-c3ccc(N(c4ccc5c(ccc6ccccc65)c4)c4ccc5ccc6ccccc6c5c4)cc3)cccc2c1 ; c1ccc2c(-c3ccc(N(c4ccc5ccc6ccccc6c5c4)c4cccc5c4ccc4ccccc45)cc3)cccc2c1] 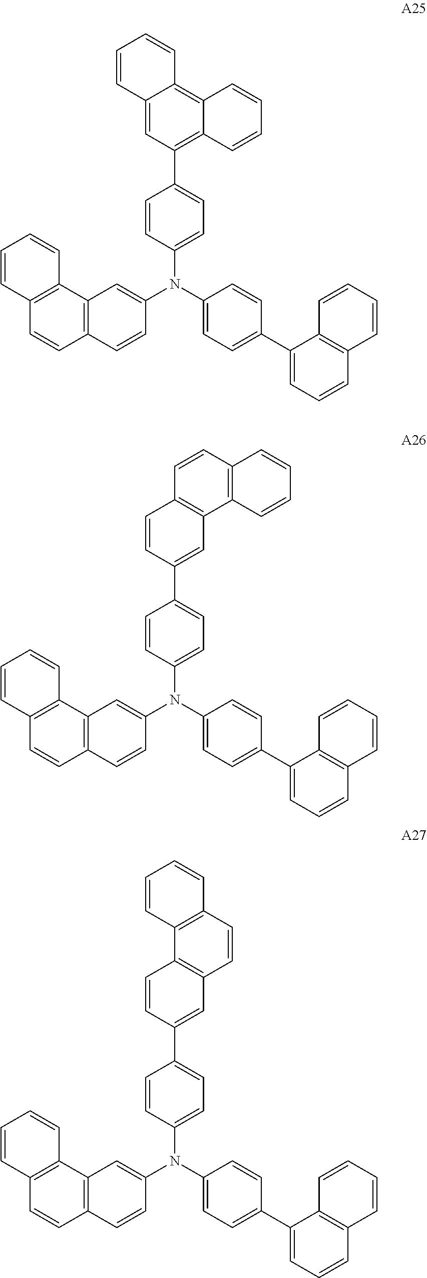 [SMILES: c1ccc2c(-c3ccc(N(c4ccc(-c5cc6ccccc6c6ccccc56)cc4)c4ccc5ccc6ccccc6c5c4)cc3)cccc2c1 ; c1ccc2c(-c3ccc(N(c4ccc(-c5ccc6ccc7ccccc7c6c5)cc4)c4ccc5ccc6ccccc6c5c4)cc3)cccc2c1 ; c1ccc2c(-c3ccc(N(c4ccc(-c5ccc6c(ccc7ccccc76)c5)cc4)c4ccc5ccc6ccccc6c5c4)cc3)cccc2c1] 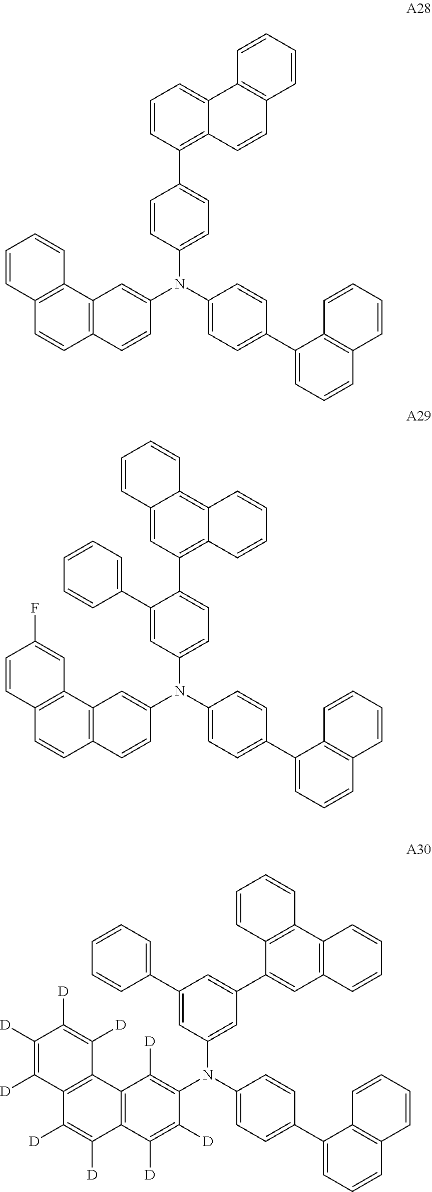 [SMILES: c1ccc2c(-c3ccc(N(c4ccc(-c5cccc6c5ccc5ccccc56)cc4)c4ccc5ccc6ccccc6c5c4)cc3)cccc2c1 ; Fc1ccc2ccc3ccc(N(c4ccc(-c5cccc6ccccc56)cc4)c4ccc(-c5cc6ccccc6c6ccccc56)c(-c5ccccc5)c4)cc3c2c1 ; [2H]c1c([2H])c([2H])c2c(c1[2H])c([2H])c([2H])c1c([2H])c([2H])c(N(c3ccc(-c4cccc5ccccc45)cc3)c3cc(-c4ccccc4)cc(-c4cc5ccccc5c5ccccc45)c3)c([2H])c12] 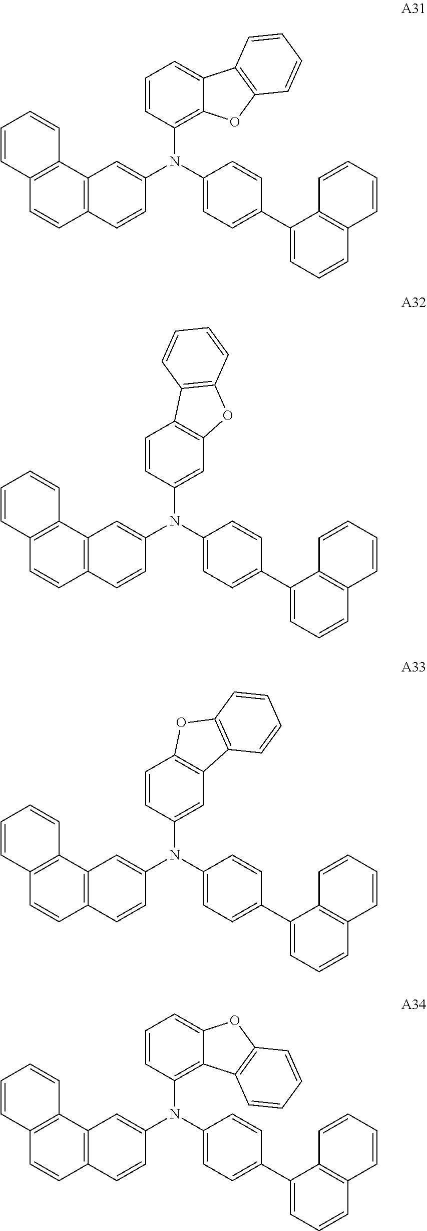 [SMILES: c1ccc2c(-c3ccc(N(c4ccc5ccc6ccccc6c5c4)c4cccc5c4oc4ccccc45)cc3)cccc2c1 ; c1ccc2c(-c3ccc(N(c4ccc5c(c4)oc4ccccc45)c4ccc5ccc6ccccc6c5c4)cc3)cccc2c1 ; c1ccc2c(-c3ccc(N(c4ccc5ccc6ccccc6c5c4)c4ccc5oc6ccccc6c5c4)cc3)cccc2c1 ; c1ccc2c(-c3ccc(N(c4ccc5ccc6ccccc6c5c4)c4cccc5oc6ccccc6c45)cc3)cccc2c1] 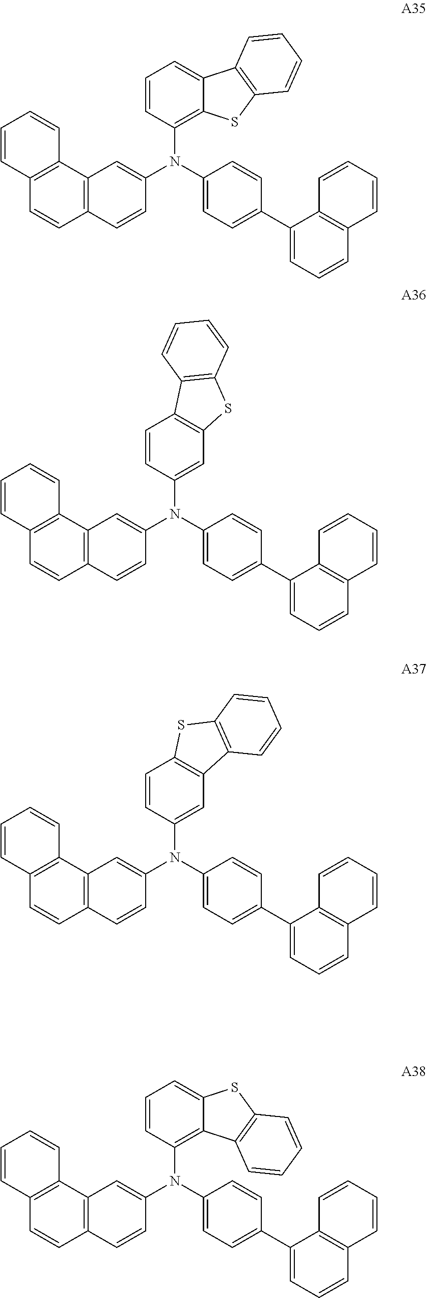 [SMILES: c1ccc2c(-c3ccc(N(c4ccc5ccc6ccccc6c5c4)c4cccc5c4sc4ccccc45)cc3)cccc2c1 ; c1ccc2c(-c3ccc(N(c4ccc5c(c4)sc4ccccc45)c4ccc5ccc6ccccc6c5c4)cc3)cccc2c1 ; c1ccc2c(-c3ccc(N(c4ccc5ccc6ccccc6c5c4)c4ccc5sc6ccccc6c5c4)cc3)cccc2c1 ; c1ccc2c(-c3ccc(N(c4ccc5ccc6ccccc6c5c4)c4cccc5sc6ccccc6c45)cc3)cccc2c1] 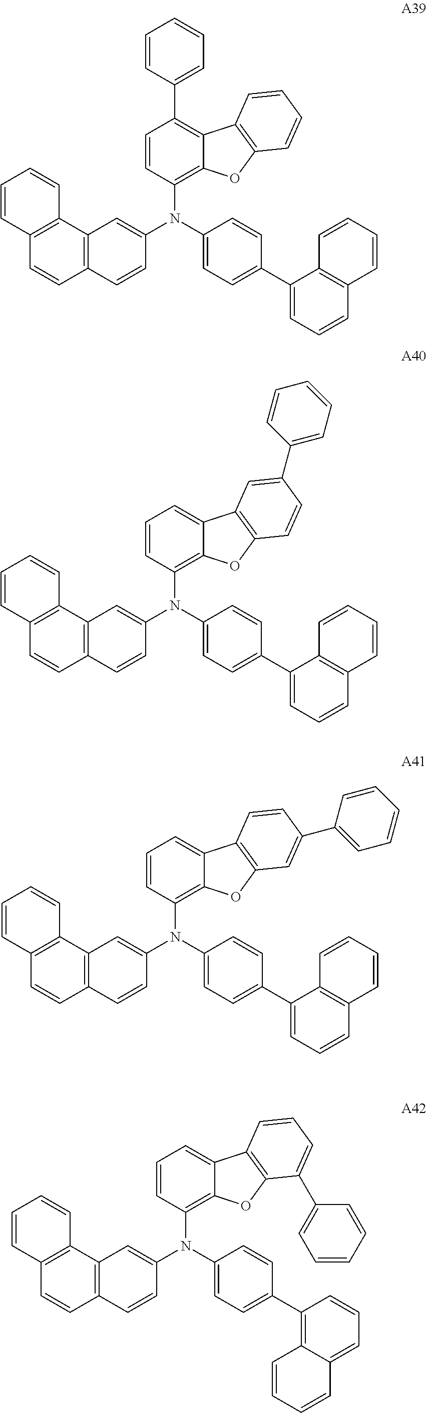 [SMILES: c1ccc(-c2ccc(N(c3ccc(-c4cccc5ccccc45)cc3)c3ccc4ccc5ccccc5c4c3)c3oc4ccccc4c23)cc1 ; c1ccc(-c2ccc3oc4c(N(c5ccc(-c6cccc7ccccc67)cc5)c5ccc6ccc7ccccc7c6c5)cccc4c3c2)cc1 ; c1ccc(-c2ccc3c(c2)oc2c(N(c4ccc(-c5cccc6ccccc56)cc4)c4ccc5ccc6ccccc6c5c4)cccc23)cc1 ; c1ccc(-c2cccc3c2oc2c(N(c4ccc(-c5cccc6ccccc56)cc4)c4ccc5ccc6ccccc6c5c4)cccc23)cc1] 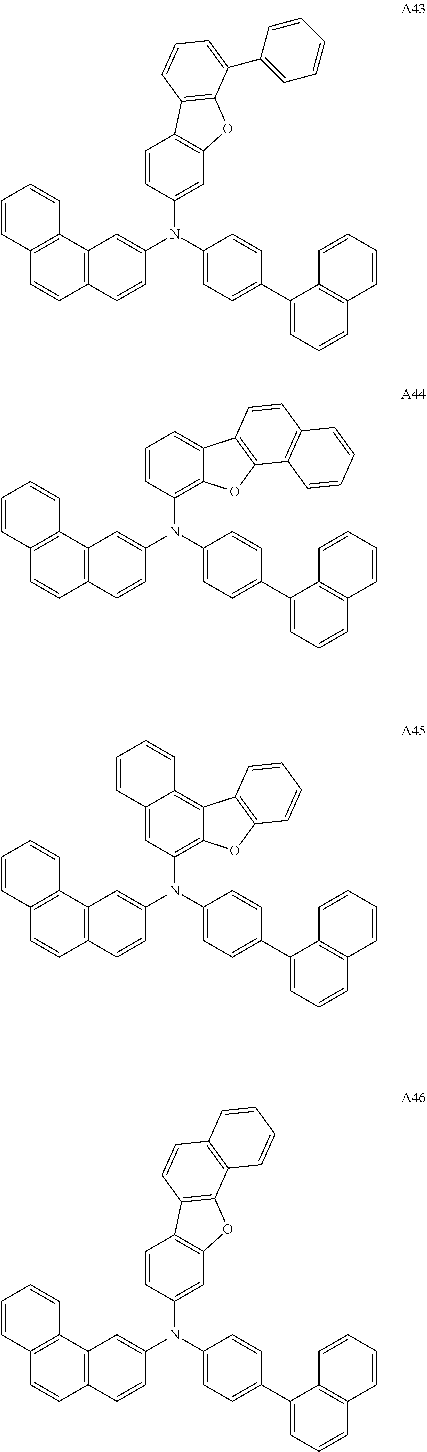 [SMILES: c1ccc(-c2cccc3c2oc2cc(N(c4ccc(-c5cccc6ccccc56)cc4)c4ccc5ccc6ccccc6c5c4)ccc23)cc1 ; c1ccc2c(-c3ccc(N(c4ccc5ccc6ccccc6c5c4)c4cccc5c4oc4c6ccccc6ccc54)cc3)cccc2c1 ; c1ccc2c(-c3ccc(N(c4ccc5ccc6ccccc6c5c4)c4cc5ccccc5c5c4oc4ccccc45)cc3)cccc2c1 ; c1ccc2c(-c3ccc(N(c4ccc5c(c4)oc4c6ccccc6ccc54)c4ccc5ccc6ccccc6c5c4)cc3)cccc2c1] 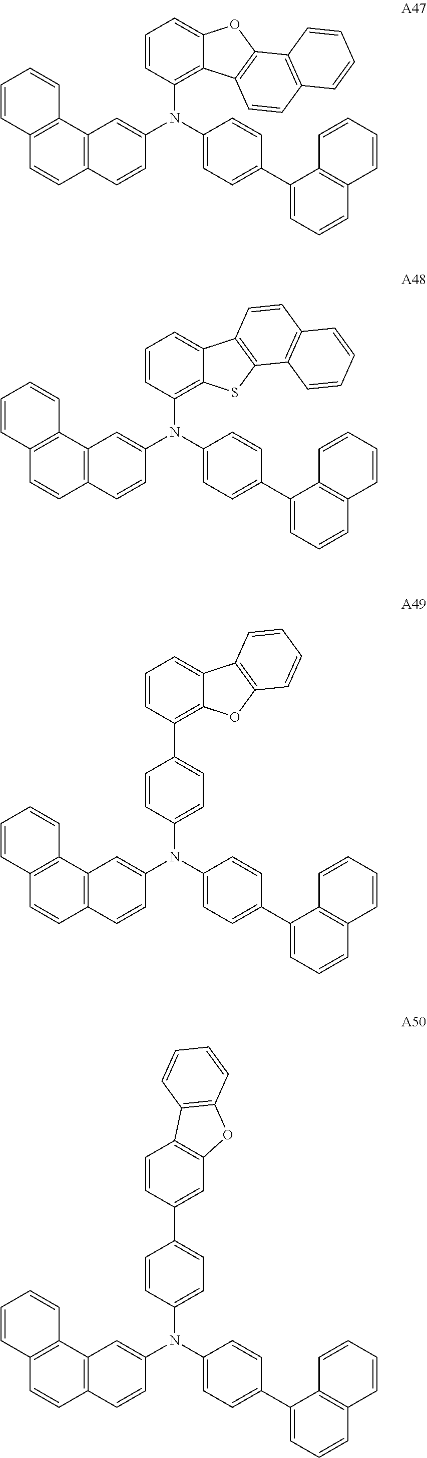 [SMILES: c1ccc2c(-c3ccc(N(c4ccc5ccc6ccccc6c5c4)c4cccc5oc6c7ccccc7ccc6c45)cc3)cccc2c1 ; c1ccc2c(-c3ccc(N(c4ccc5ccc6ccccc6c5c4)c4cccc5c4sc4c6ccccc6ccc54)cc3)cccc2c1 ; c1ccc2c(-c3ccc(N(c4ccc(-c5cccc6c5oc5ccccc56)cc4)c4ccc5ccc6ccccc6c5c4)cc3)cccc2c1 ; c1ccc2c(-c3ccc(N(c4ccc(-c5ccc6c(c5)oc5ccccc56)cc4)c4ccc5ccc6ccccc6c5c4)cc3)cccc2c1] 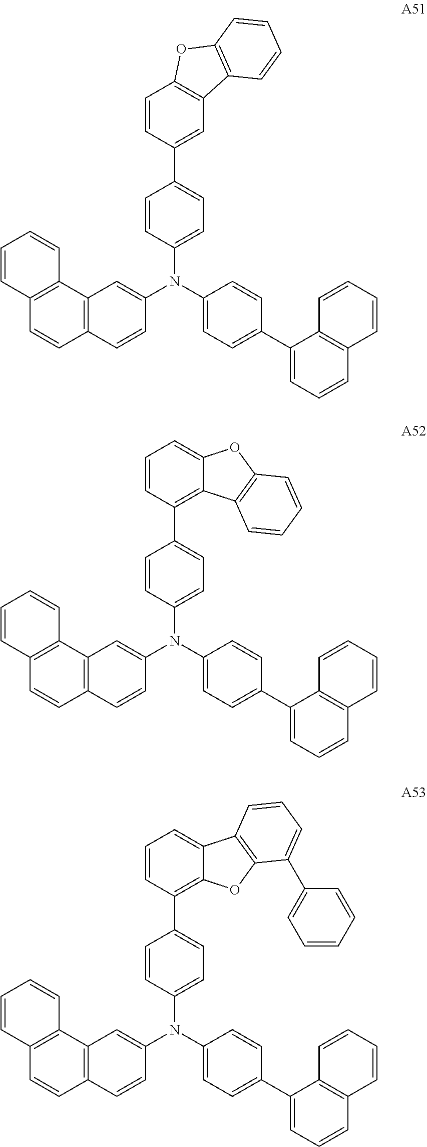 [SMILES: c1ccc2c(-c3ccc(N(c4ccc(-c5ccc6oc7ccccc7c6c5)cc4)c4ccc5ccc6ccccc6c5c4)cc3)cccc2c1 ; c1ccc2c(-c3ccc(N(c4ccc(-c5cccc6oc7ccccc7c56)cc4)c4ccc5ccc6ccccc6c5c4)cc3)cccc2c1 ; c1ccc(-c2cccc3c2oc2c(-c4ccc(N(c5ccc(-c6cccc7ccccc67)cc5)c5ccc6ccc7ccccc7c6c5)cc4)cccc23)cc1] 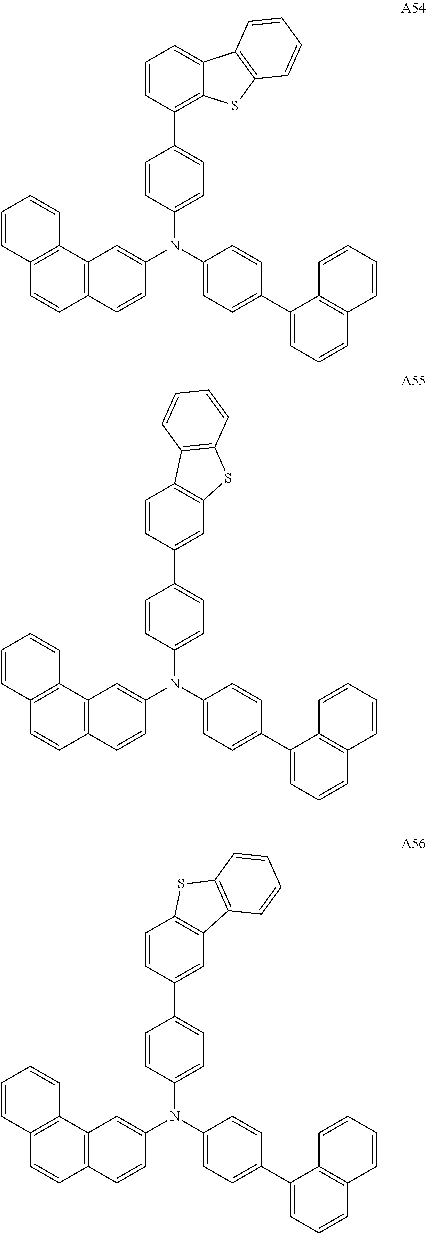 [SMILES: c1ccc2c(-c3ccc(N(c4ccc(-c5cccc6c5sc5ccccc56)cc4)c4ccc5ccc6ccccc6c5c4)cc3)cccc2c1 ; c1ccc2c(-c3ccc(N(c4ccc(-c5ccc6c(c5)sc5ccccc56)cc4)c4ccc5ccc6ccccc6c5c4)cc3)cccc2c1 ; c1ccc2c(-c3ccc(N(c4ccc(-c5ccc6sc7ccccc7c6c5)cc4)c4ccc5ccc6ccccc6c5c4)cc3)cccc2c1] 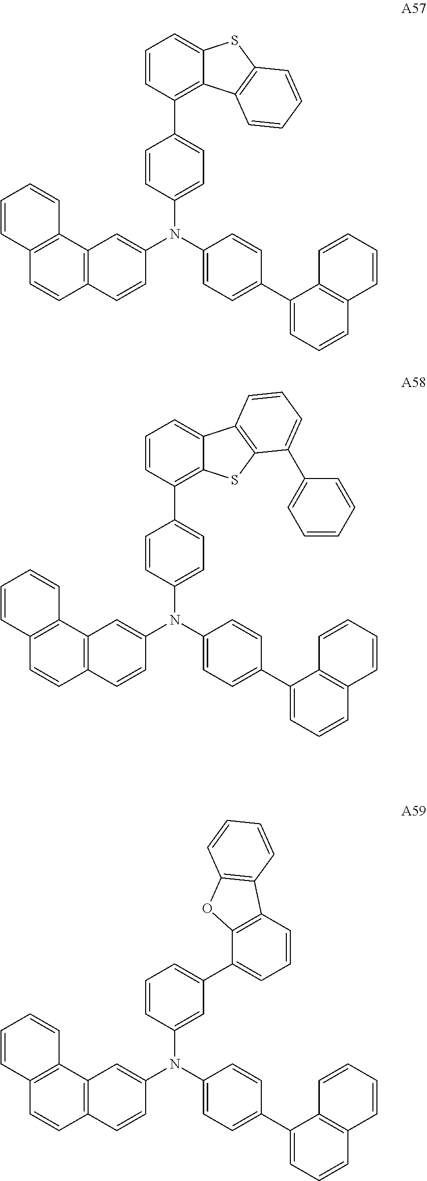 [SMILES: c1ccc2c(-c3ccc(N(c4ccc(-c5cccc6sc7ccccc7c56)cc4)c4ccc5ccc6ccccc6c5c4)cc3)cccc2c1 ; c1ccc(-c2cccc3c2sc2c(-c4ccc(N(c5ccc(-c6cccc7ccccc67)cc5)c5ccc6ccc7ccccc7c6c5)cc4)cccc23)cc1 ; c1cc(-c2cccc3c2oc2ccccc23)cc(N(c2ccc(-c3cccc4ccccc34)cc2)c2ccc3ccc4ccccc4c3c2)c1] 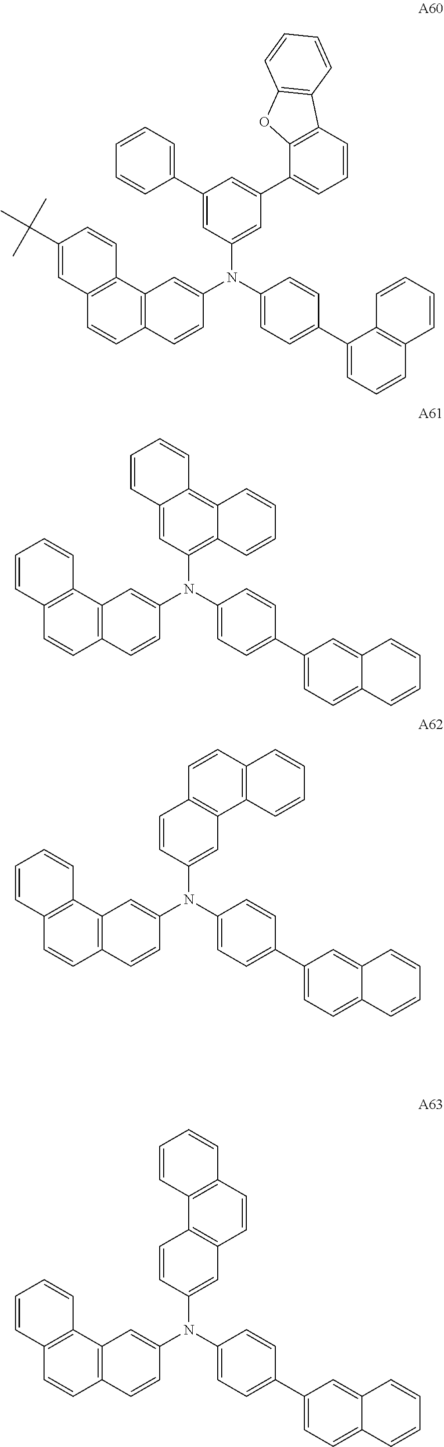 [SMILES: CC(C)(C)c1ccc2c(ccc3ccc(N(c4ccc(-c5cccc6ccccc56)cc4)c4cc(-c5ccccc5)cc(-c5cccc6c5oc5ccccc56)c4)cc32)c1 ; c1ccc2cc(-c3ccc(N(c4ccc5ccc6ccccc6c5c4)c4cc5ccccc5c5ccccc45)cc3)ccc2c1 ; c1ccc2cc(-c3ccc(N(c4ccc5ccc6ccccc6c5c4)c4ccc5ccc6ccccc6c5c4)cc3)ccc2c1 ; c1ccc2cc(-c3ccc(N(c4ccc5c(ccc6ccccc65)c4)c4ccc5ccc6ccccc6c5c4)cc3)ccc2c1] 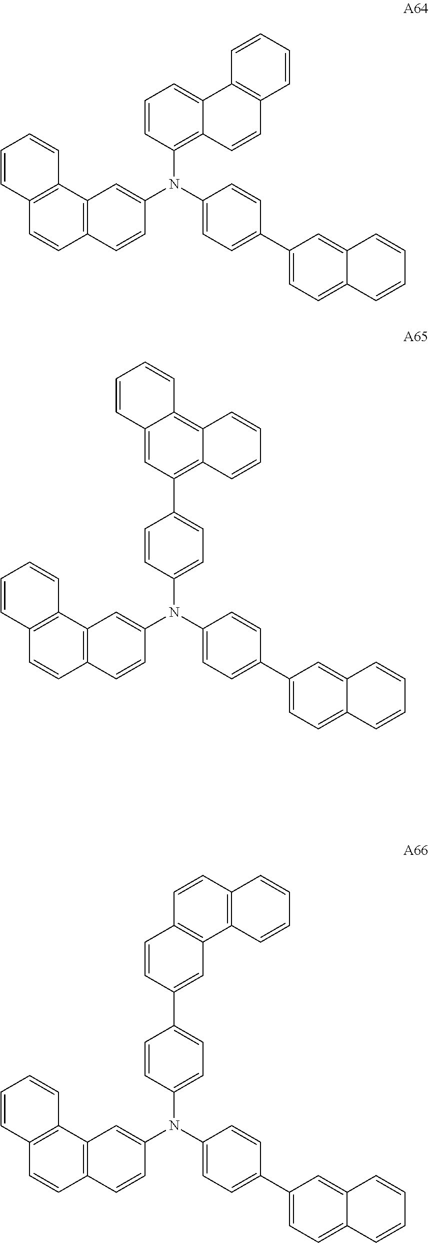 [SMILES: c1ccc2cc(-c3ccc(N(c4ccc5ccc6ccccc6c5c4)c4cccc5c4ccc4ccccc45)cc3)ccc2c1 ; c1ccc2cc(-c3ccc(N(c4ccc(-c5cc6ccccc6c6ccccc56)cc4)c4ccc5ccc6ccccc6c5c4)cc3)ccc2c1 ; c1ccc2cc(-c3ccc(N(c4ccc(-c5ccc6ccc7ccccc7c6c5)cc4)c4ccc5ccc6ccccc6c5c4)cc3)ccc2c1] 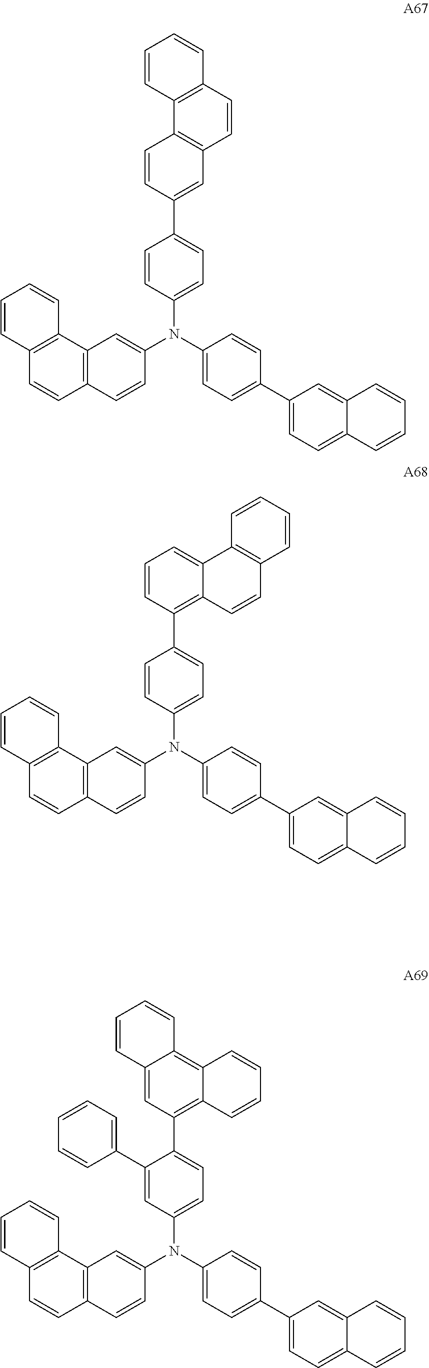 [SMILES: c1ccc2cc(-c3ccc(N(c4ccc(-c5ccc6c(ccc7ccccc76)c5)cc4)c4ccc5ccc6ccccc6c5c4)cc3)ccc2c1 ; c1ccc2cc(-c3ccc(N(c4ccc(-c5cccc6c5ccc5ccccc56)cc4)c4ccc5ccc6ccccc6c5c4)cc3)ccc2c1 ; c1ccc(-c2cc(N(c3ccc(-c4ccc5ccccc5c4)cc3)c3ccc4ccc5ccccc5c4c3)ccc2-c2cc3ccccc3c3ccccc23)cc1] 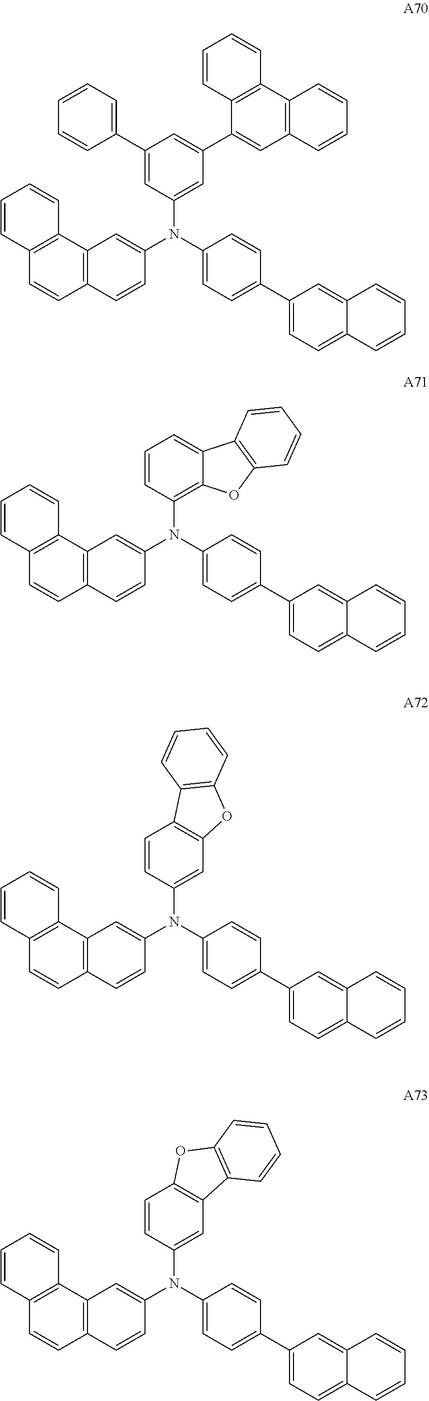 [SMILES: c1ccc(-c2cc(-c3cc4ccccc4c4ccccc34)cc(N(c3ccc(-c4ccc5ccccc5c4)cc3)c3ccc4ccc5ccccc5c4c3)c2)cc1 ; c1ccc2cc(-c3ccc(N(c4ccc5ccc6ccccc6c5c4)c4cccc5c4oc4ccccc45)cc3)ccc2c1 ; c1ccc2cc(-c3ccc(N(c4ccc5c(c4)oc4ccccc45)c4ccc5ccc6ccccc6c5c4)cc3)ccc2c1 ; c1ccc2cc(-c3ccc(N(c4ccc5ccc6ccccc6c5c4)c4ccc5oc6ccccc6c5c4)cc3)ccc2c1] 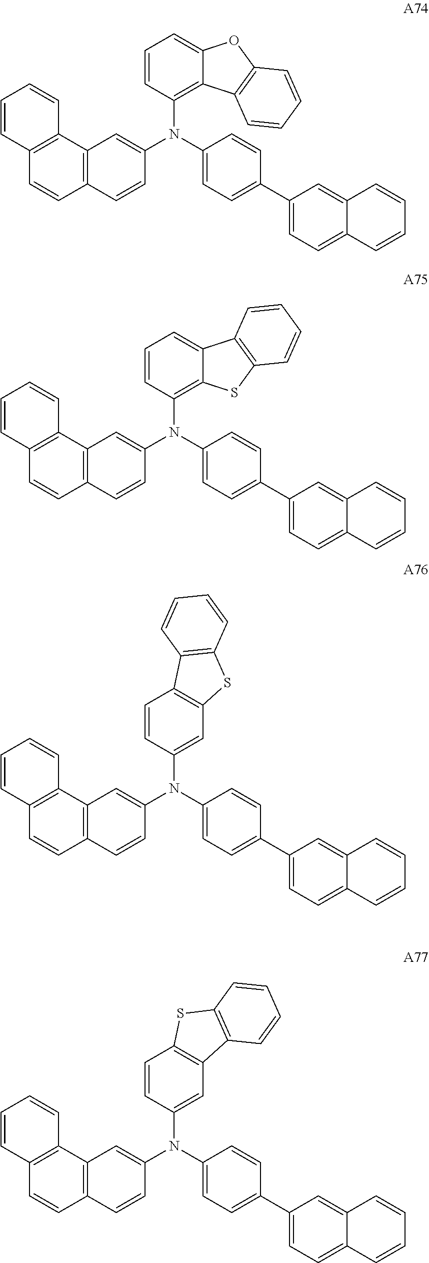 [SMILES: c1ccc2cc(-c3ccc(N(c4ccc5ccc6ccccc6c5c4)c4cccc5oc6ccccc6c45)cc3)ccc2c1 ; c1ccc2cc(-c3ccc(N(c4ccc5ccc6ccccc6c5c4)c4cccc5c4sc4ccccc45)cc3)ccc2c1 ; c1ccc2cc(-c3ccc(N(c4ccc5c(c4)sc4ccccc45)c4ccc5ccc6ccccc6c5c4)cc3)ccc2c1 ; c1ccc2cc(-c3ccc(N(c4ccc5ccc6ccccc6c5c4)c4ccc5sc6ccccc6c5c4)cc3)ccc2c1] 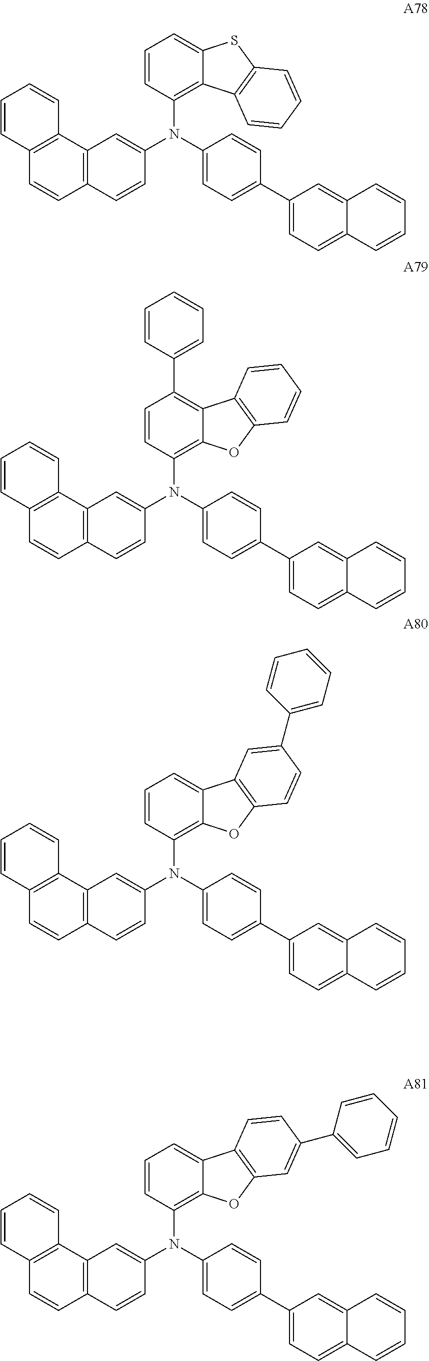 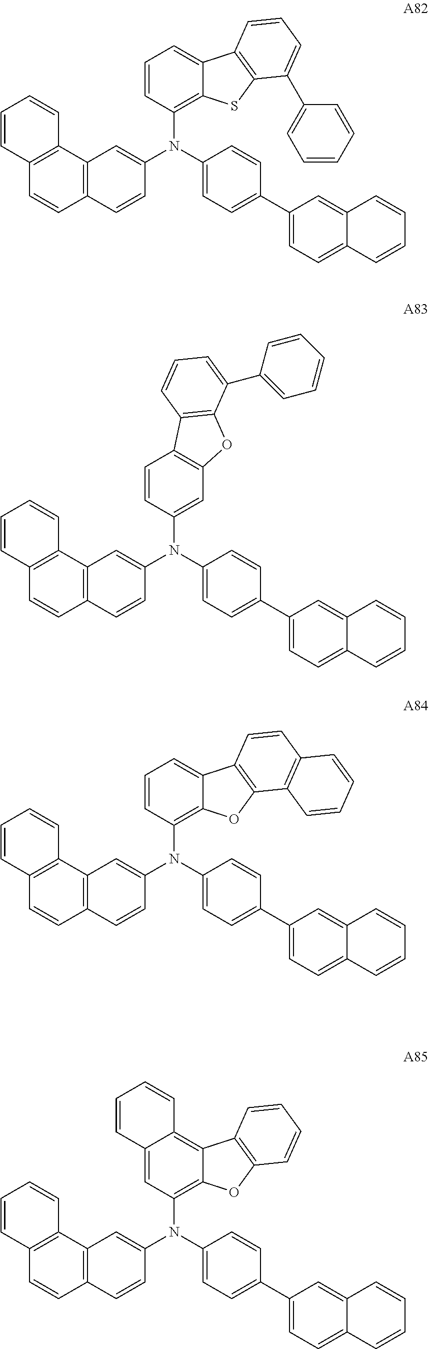 [SMILES: c1ccc(-c2cccc3c2sc2c(N(c4ccc(-c5ccc6ccccc6c5)cc4)c4ccc5ccc6ccccc6c5c4)cccc23)cc1 ; c1ccc(-c2cccc3c2oc2cc(N(c4ccc(-c5ccc6ccccc6c5)cc4)c4ccc5ccc6ccccc6c5c4)ccc23)cc1 ; c1ccc2cc(-c3ccc(N(c4ccc5ccc6ccccc6c5c4)c4cccc5c4oc4c6ccccc6ccc54)cc3)ccc2c1 ; c1ccc2cc(-c3ccc(N(c4ccc5ccc6ccccc6c5c4)c4cc5ccccc5c5c4oc4ccccc45)cc3)ccc2c1] 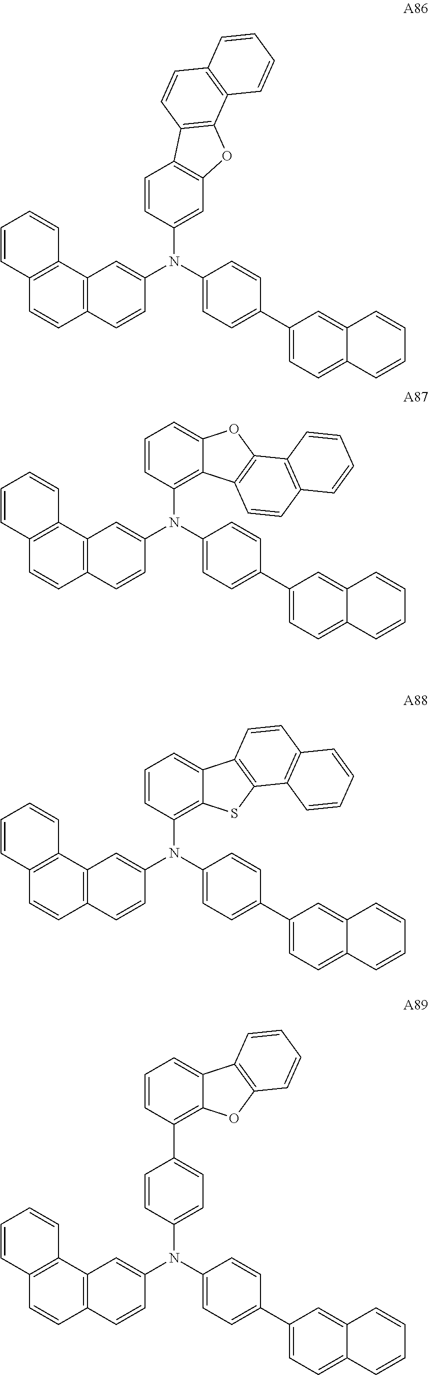 [SMILES: c1ccc2cc(-c3ccc(N(c4ccc5c(c4)oc4c6ccccc6ccc54)c4ccc5ccc6ccccc6c5c4)cc3)ccc2c1 ; c1ccc2cc(-c3ccc(N(c4ccc5ccc6ccccc6c5c4)c4cccc5oc6c7ccccc7ccc6c45)cc3)ccc2c1 ; c1ccc2cc(-c3ccc(N(c4ccc5ccc6ccccc6c5c4)c4cccc5c4sc4c6ccccc6ccc54)cc3)ccc2c1 ; c1ccc2cc(-c3ccc(N(c4ccc(-c5cccc6c5oc5ccccc56)cc4)c4ccc5ccc6ccccc6c5c4)cc3)ccc2c1] 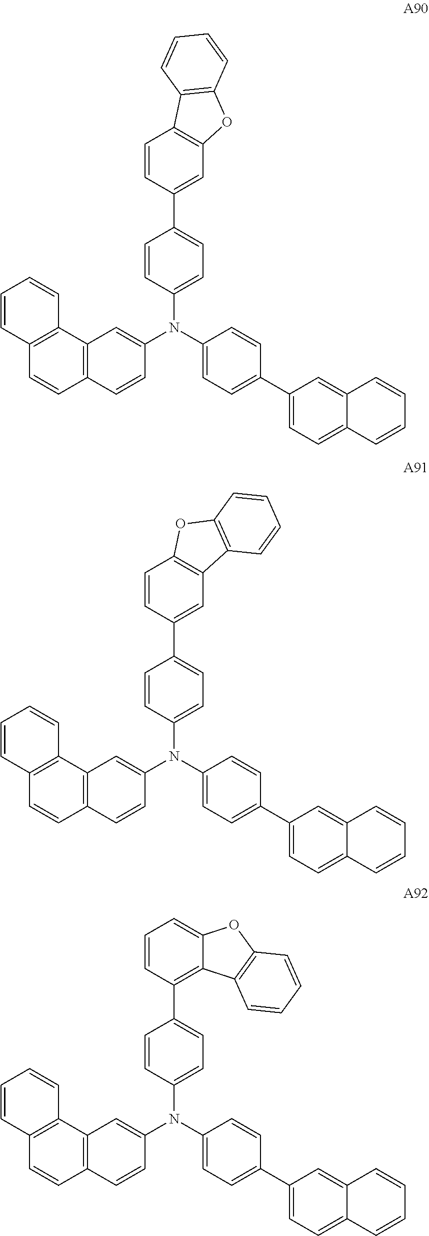 [SMILES: c1ccc2cc(-c3ccc(N(c4ccc(-c5ccc6c(c5)oc5ccccc56)cc4)c4ccc5ccc6ccccc6c5c4)cc3)ccc2c1 ; c1ccc2cc(-c3ccc(N(c4ccc(-c5ccc6oc7ccccc7c6c5)cc4)c4ccc5ccc6ccccc6c5c4)cc3)ccc2c1 ; c1ccc2cc(-c3ccc(N(c4ccc(-c5cccc6oc7ccccc7c56)cc4)c4ccc5ccc6ccccc6c5c4)cc3)ccc2c1] 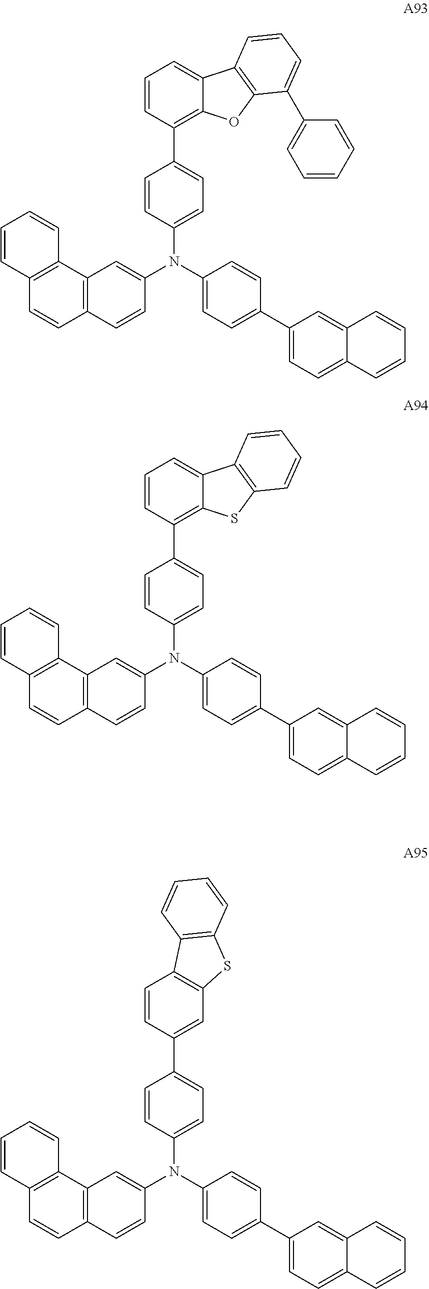 [SMILES: c1ccc(-c2cccc3c2oc2c(-c4ccc(N(c5ccc(-c6ccc7ccccc7c6)cc5)c5ccc6ccc7ccccc7c6c5)cc4)cccc23)cc1 ; c1ccc2cc(-c3ccc(N(c4ccc(-c5cccc6c5sc5ccccc56)cc4)c4ccc5ccc6ccccc6c5c4)cc3)ccc2c1 ; c1ccc2cc(-c3ccc(N(c4ccc(-c5ccc6c(c5)sc5ccccc56)cc4)c4ccc5ccc6ccccc6c5c4)cc3)ccc2c1] 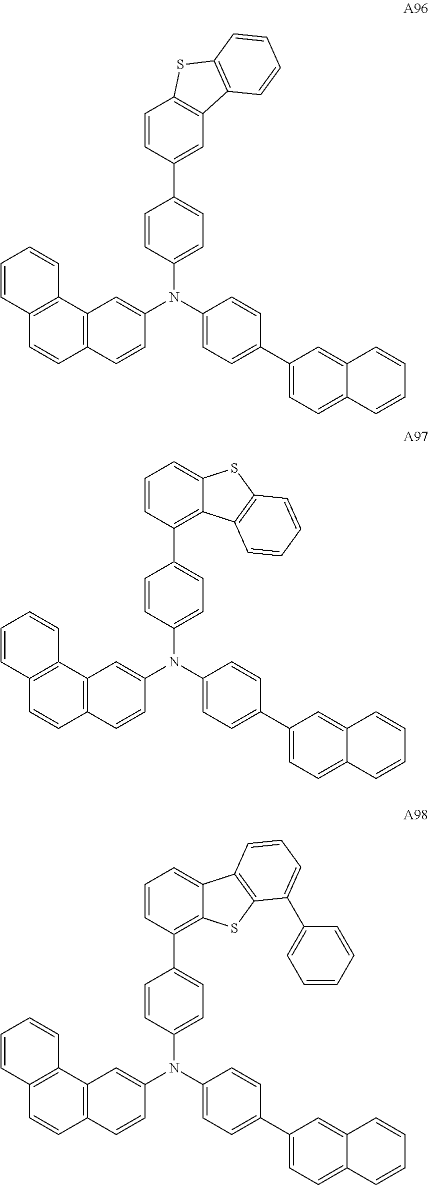 [SMILES: c1ccc2cc(-c3ccc(N(c4ccc(-c5ccc6sc7ccccc7c6c5)cc4)c4ccc5ccc6ccccc6c5c4)cc3)ccc2c1 ; c1ccc2cc(-c3ccc(N(c4ccc(-c5cccc6sc7ccccc7c56)cc4)c4ccc5ccc6ccccc6c5c4)cc3)ccc2c1 ; c1ccc(-c2cccc3c2sc2c(-c4ccc(N(c5ccc(-c6ccc7ccccc7c6)cc5)c5ccc6ccc7ccccc7c6c5)cc4)cccc23)cc1] 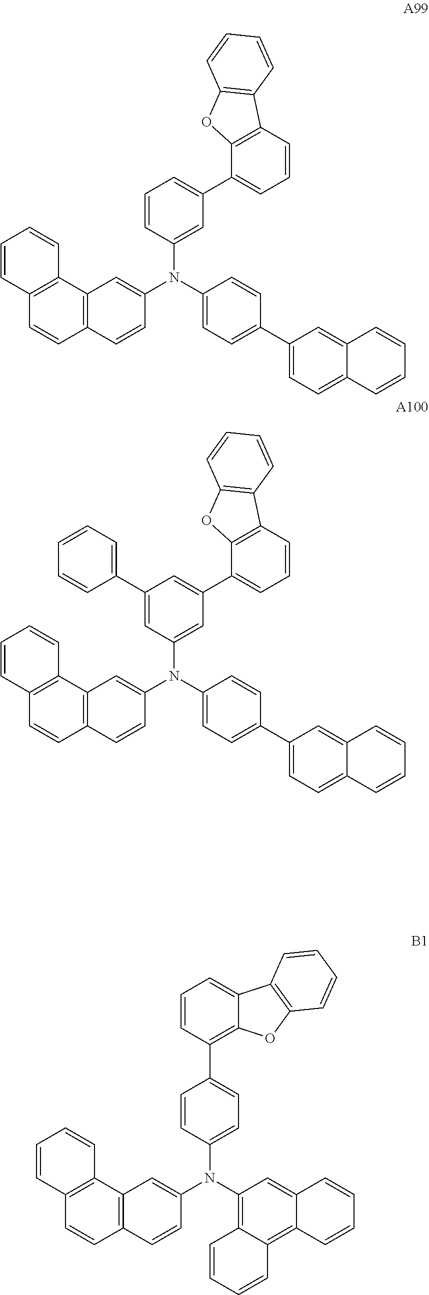 [SMILES: c1cc(-c2cccc3c2oc2ccccc23)cc(N(c2ccc(-c3ccc4ccccc4c3)cc2)c2ccc3ccc4ccccc4c3c2)c1 ; c1ccc(-c2cc(-c3cccc4c3oc3ccccc34)cc(N(c3ccc(-c4ccc5ccccc5c4)cc3)c3ccc4ccc5ccccc5c4c3)c2)cc1 ; c1ccc2c(c1)ccc1ccc(N(c3ccc(-c4cccc5c4oc4ccccc45)cc3)c3cc4ccccc4c4ccccc34)cc12] 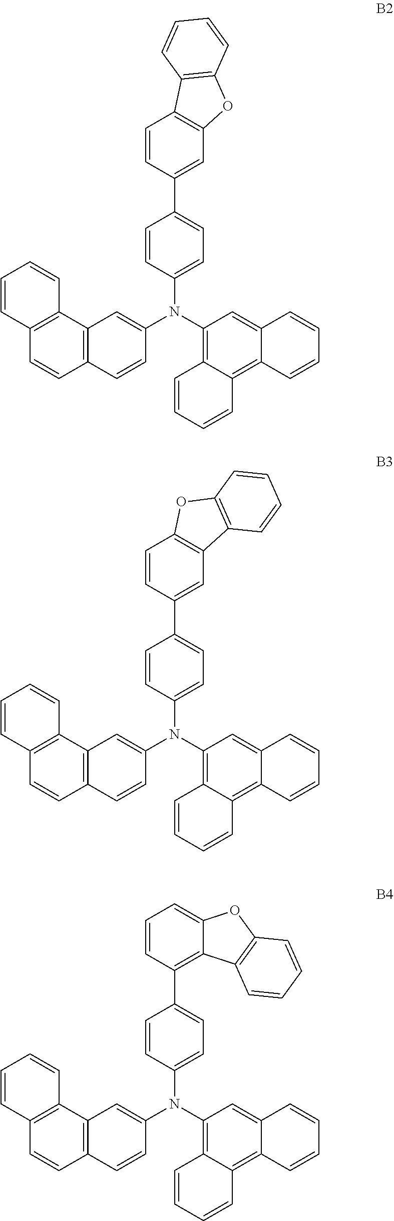 [SMILES: c1ccc2c(c1)ccc1ccc(N(c3ccc(-c4ccc5c(c4)oc4ccccc45)cc3)c3cc4ccccc4c4ccccc34)cc12 ; c1ccc2c(c1)ccc1ccc(N(c3ccc(-c4ccc5oc6ccccc6c5c4)cc3)c3cc4ccccc4c4ccccc34)cc12 ; c1ccc2c(c1)ccc1ccc(N(c3ccc(-c4cccc5oc6ccccc6c45)cc3)c3cc4ccccc4c4ccccc34)cc12] 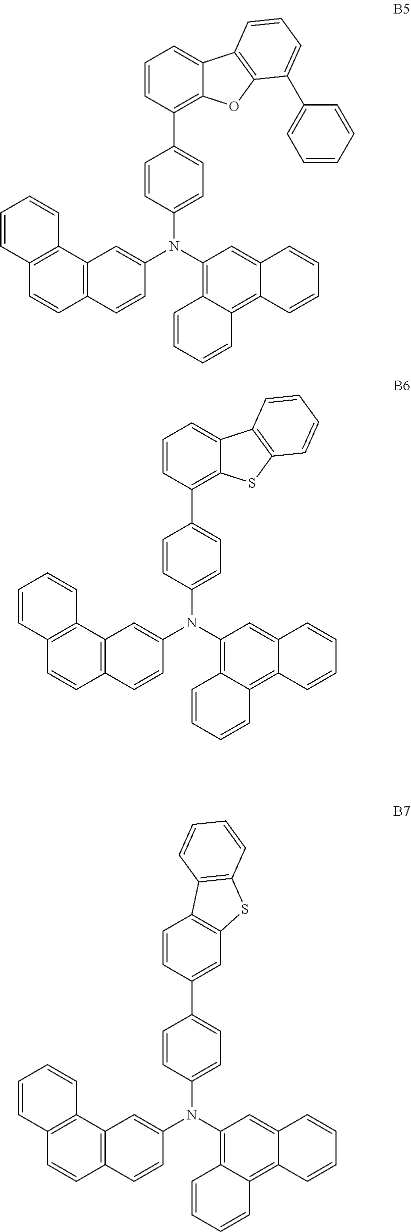 [SMILES: c1ccc(-c2cccc3c2oc2c(-c4ccc(N(c5ccc6ccc7ccccc7c6c5)c5cc6ccccc6c6ccccc56)cc4)cccc23)cc1 ; c1ccc2c(c1)ccc1ccc(N(c3ccc(-c4cccc5c4sc4ccccc45)cc3)c3cc4ccccc4c4ccccc34)cc12 ; c1ccc2c(c1)ccc1ccc(N(c3ccc(-c4ccc5c(c4)sc4ccccc45)cc3)c3cc4ccccc4c4ccccc34)cc12] 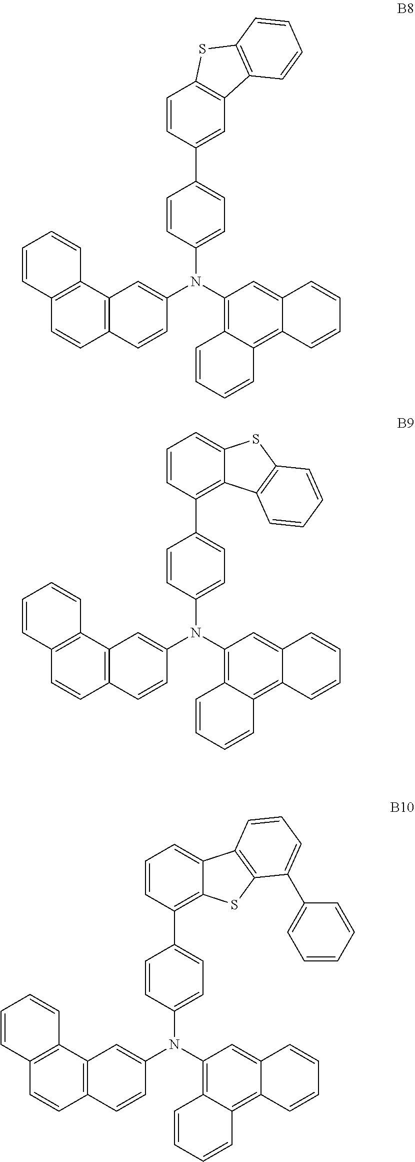 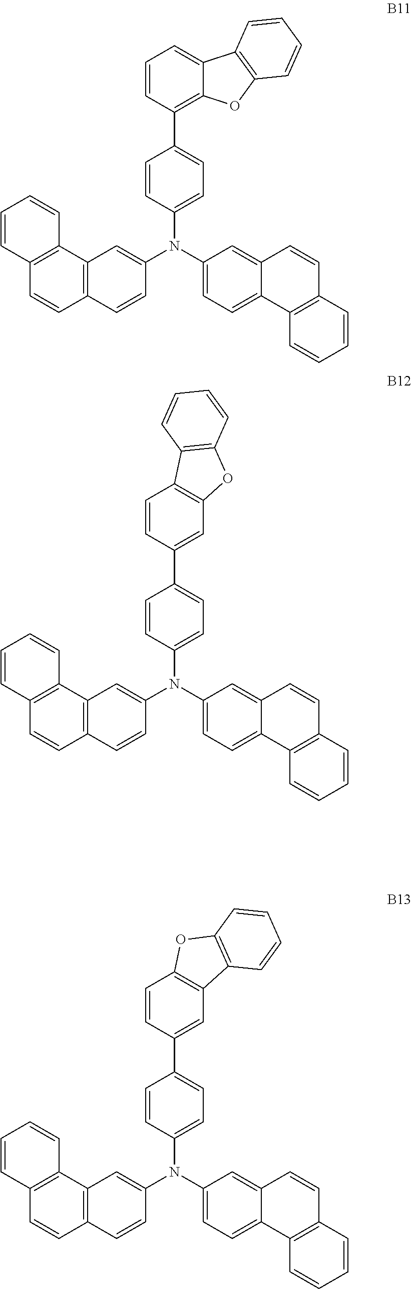 [SMILES: c1ccc2c(c1)ccc1cc(N(c3ccc(-c4cccc5c4oc4ccccc45)cc3)c3ccc4ccc5ccccc5c4c3)ccc12 ; c1ccc2c(c1)ccc1cc(N(c3ccc(-c4ccc5c(c4)oc4ccccc45)cc3)c3ccc4ccc5ccccc5c4c3)ccc12 ; c1ccc2c(c1)ccc1cc(N(c3ccc(-c4ccc5oc6ccccc6c5c4)cc3)c3ccc4ccc5ccccc5c4c3)ccc12] 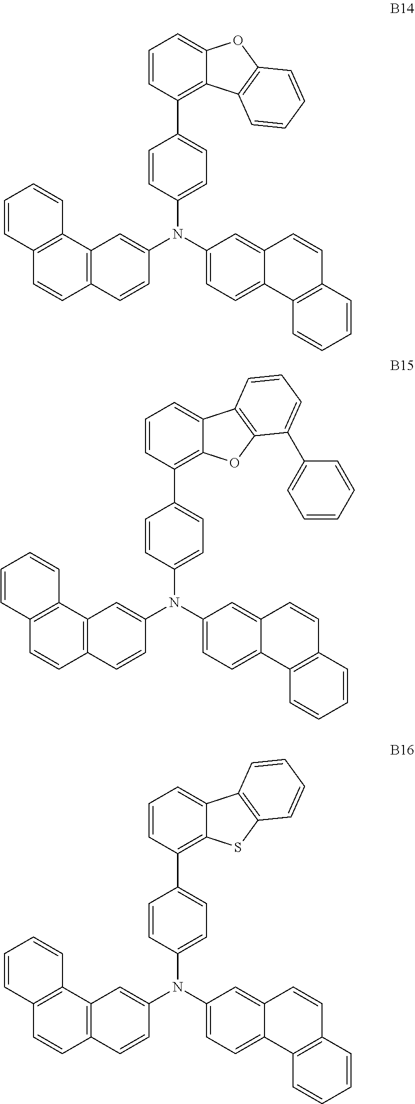 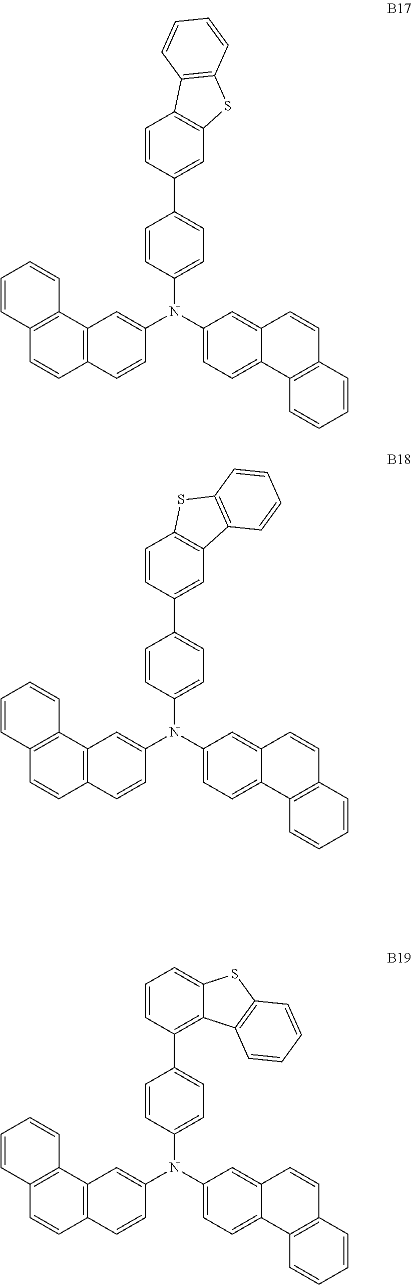 [SMILES: c1ccc2c(c1)ccc1cc(N(c3ccc(-c4ccc5c(c4)sc4ccccc45)cc3)c3ccc4ccc5ccccc5c4c3)ccc12 ; c1ccc2c(c1)ccc1cc(N(c3ccc(-c4ccc5sc6ccccc6c5c4)cc3)c3ccc4ccc5ccccc5c4c3)ccc12 ; c1ccc2c(c1)ccc1cc(N(c3ccc(-c4cccc5sc6ccccc6c45)cc3)c3ccc4ccc5ccccc5c4c3)ccc12] 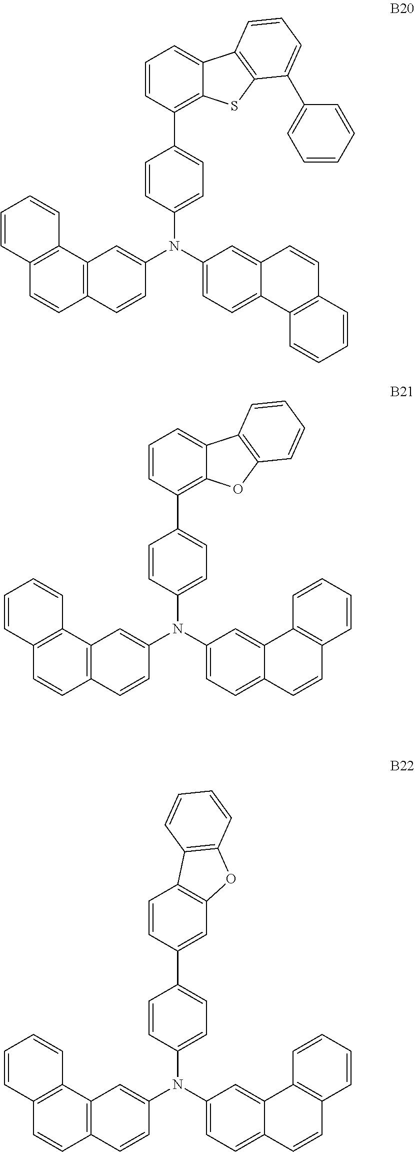 [SMILES: c1ccc(-c2cccc3c2sc2c(-c4ccc(N(c5ccc6c(ccc7ccccc76)c5)c5ccc6ccc7ccccc7c6c5)cc4)cccc23)cc1 ; c1ccc2c(c1)ccc1ccc(N(c3ccc(-c4cccc5c4oc4ccccc45)cc3)c3ccc4ccc5ccccc5c4c3)cc12 ; c1ccc2c(c1)ccc1ccc(N(c3ccc(-c4ccc5c(c4)oc4ccccc45)cc3)c3ccc4ccc5ccccc5c4c3)cc12] 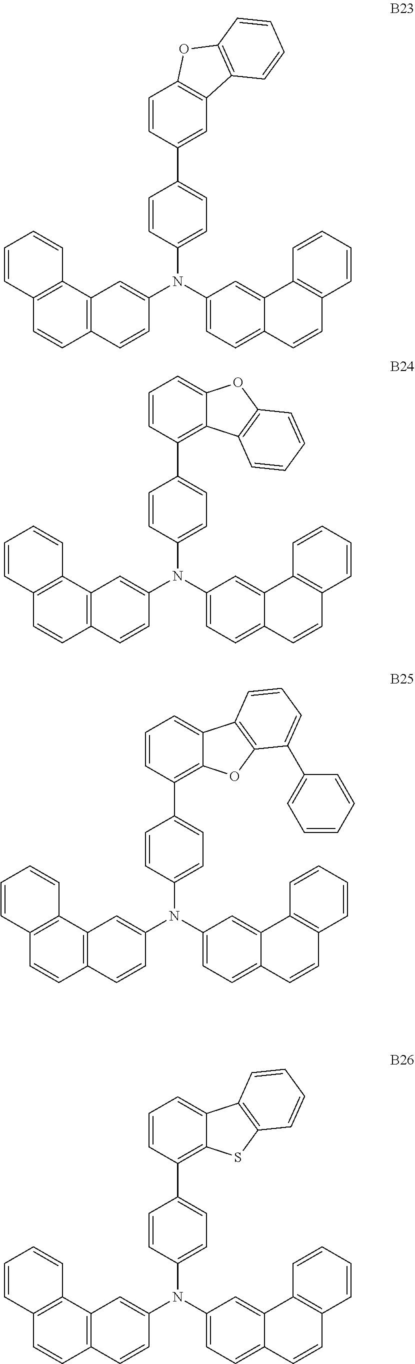 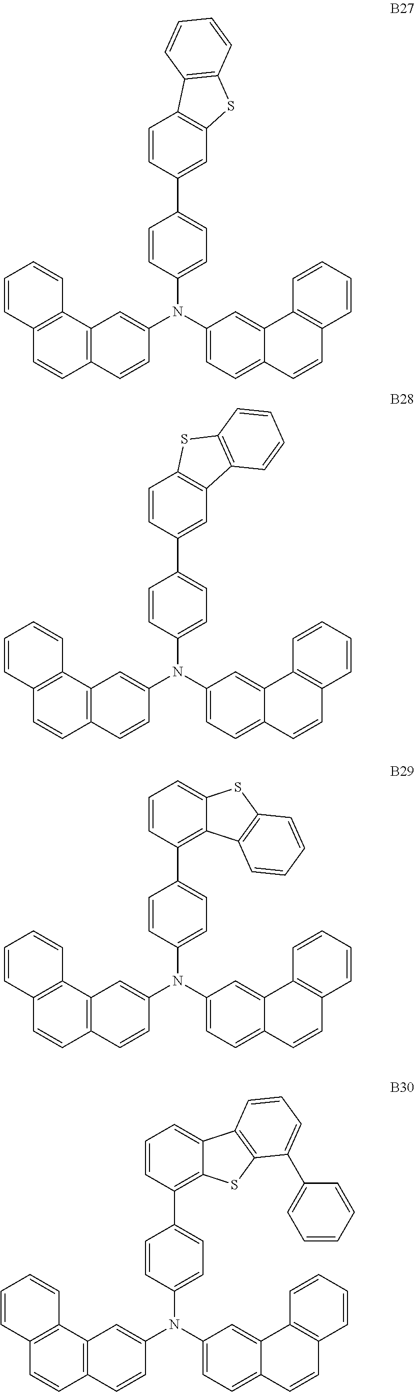 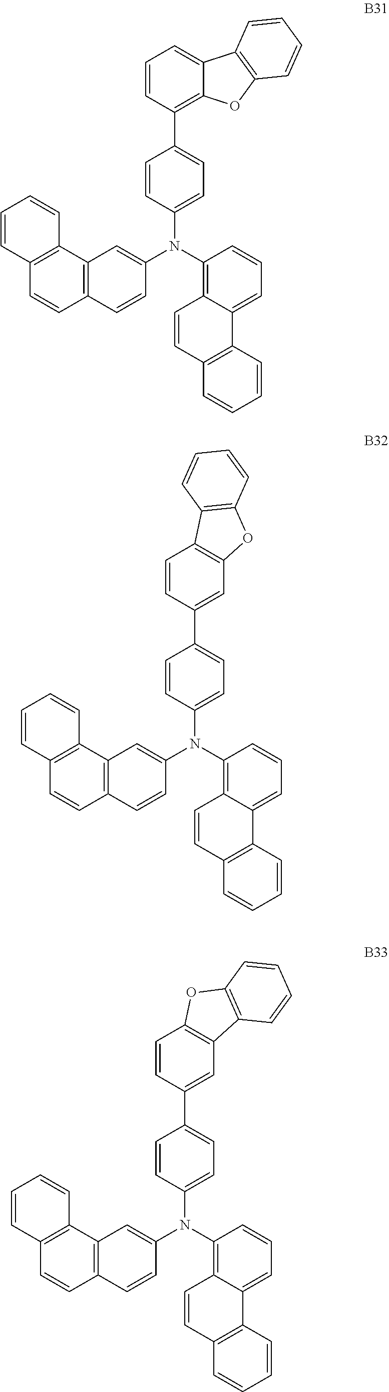 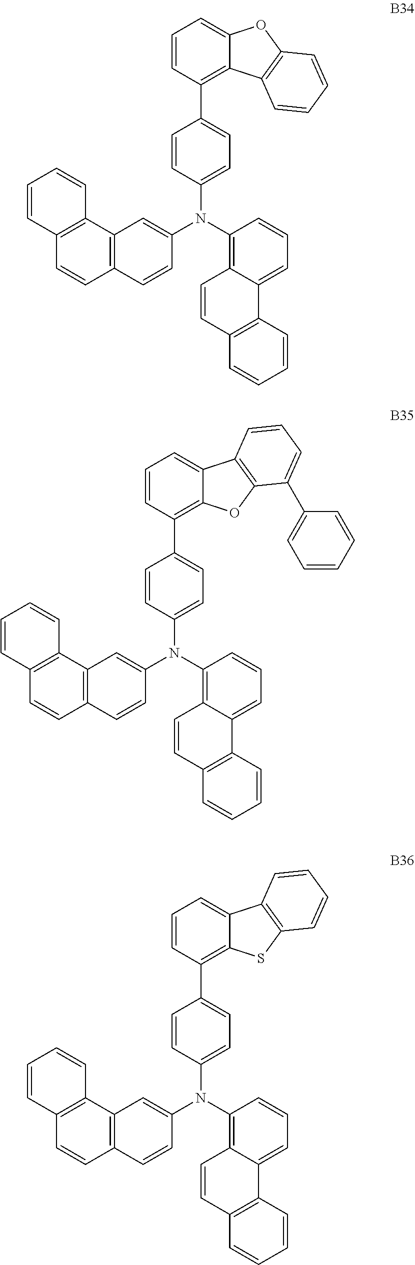 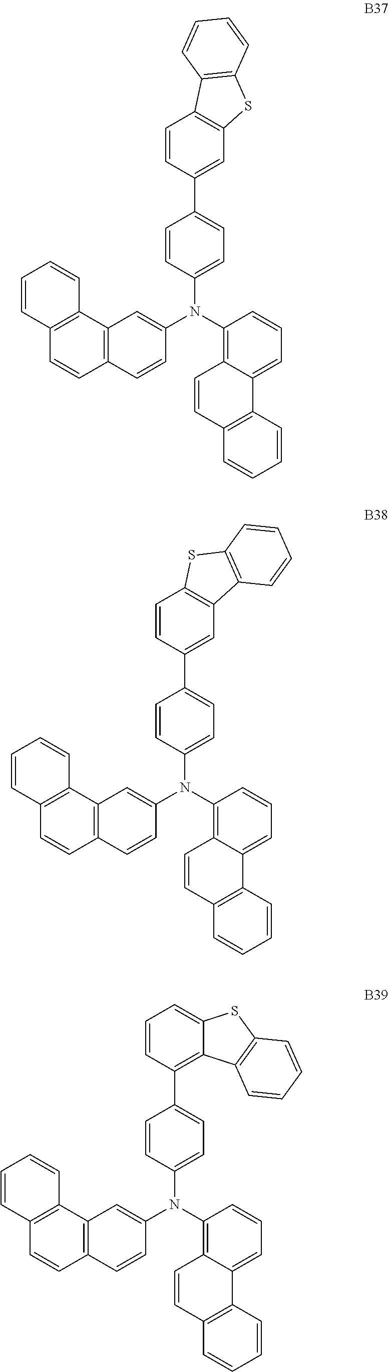 [SMILES: c1ccc2c(c1)ccc1ccc(N(c3ccc(-c4ccc5c(c4)sc4ccccc45)cc3)c3cccc4c3ccc3ccccc34)cc12 ; c1ccc2c(c1)ccc1ccc(N(c3ccc(-c4ccc5sc6ccccc6c5c4)cc3)c3cccc4c3ccc3ccccc34)cc12 ; c1ccc2c(c1)ccc1ccc(N(c3ccc(-c4cccc5sc6ccccc6c45)cc3)c3cccc4c3ccc3ccccc34)cc12] 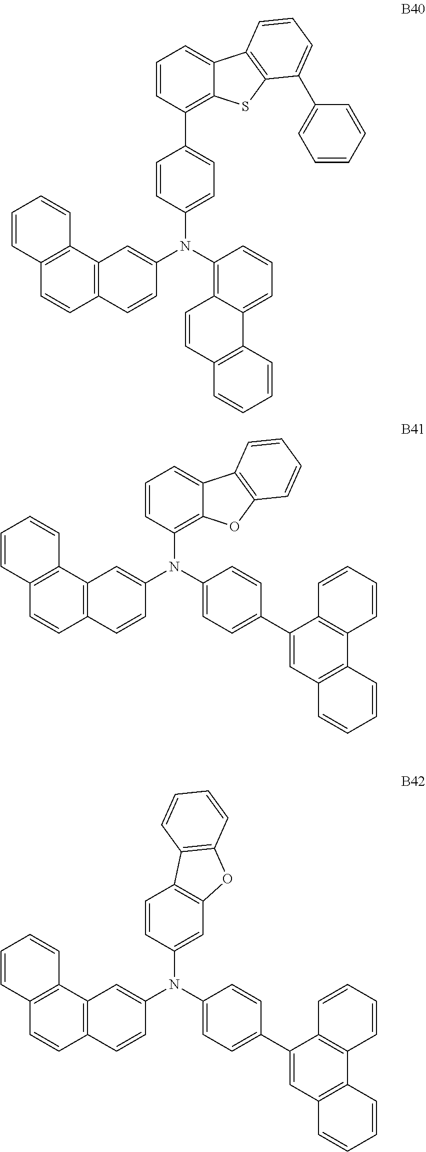 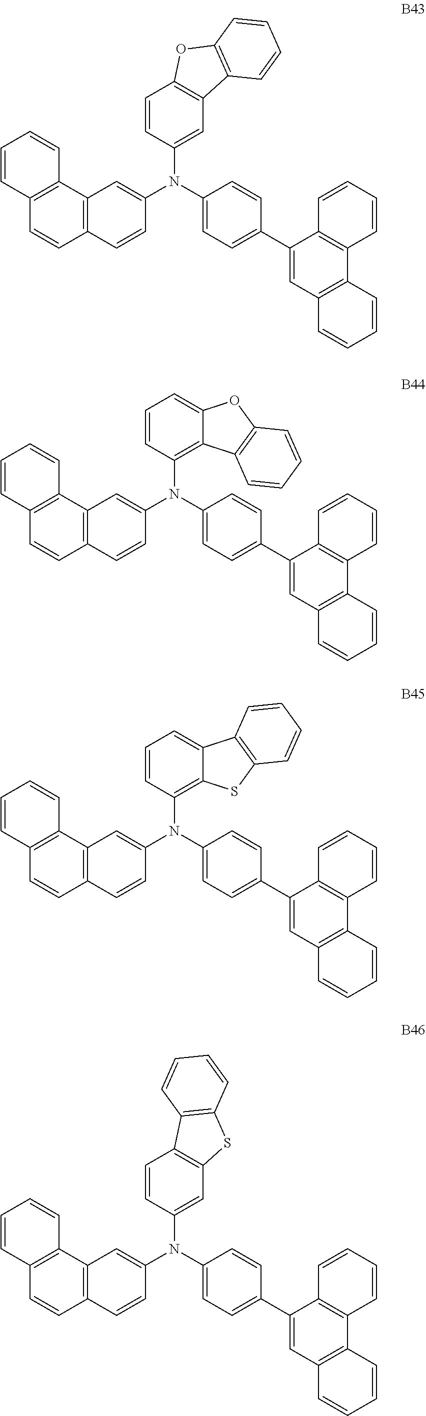 [SMILES: c1ccc2c(c1)ccc1ccc(N(c3ccc(-c4cc5ccccc5c5ccccc45)cc3)c3ccc4oc5ccccc5c4c3)cc12 ; c1ccc2c(c1)ccc1ccc(N(c3ccc(-c4cc5ccccc5c5ccccc45)cc3)c3cccc4oc5ccccc5c34)cc12 ; c1ccc2c(c1)ccc1ccc(N(c3ccc(-c4cc5ccccc5c5ccccc45)cc3)c3cccc4c3sc3ccccc34)cc12 ; c1ccc2c(c1)ccc1ccc(N(c3ccc(-c4cc5ccccc5c5ccccc45)cc3)c3ccc4c(c3)sc3ccccc34)cc12] 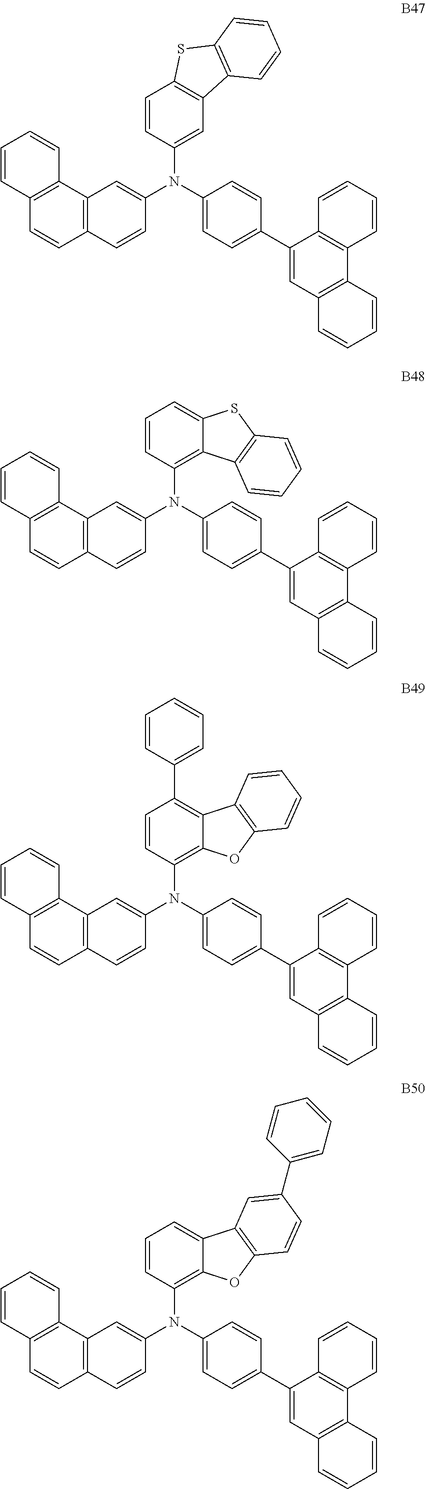 [SMILES: c1ccc2c(c1)ccc1ccc(N(c3ccc(-c4cc5ccccc5c5ccccc45)cc3)c3ccc4sc5ccccc5c4c3)cc12 ; c1ccc2c(c1)ccc1ccc(N(c3ccc(-c4cc5ccccc5c5ccccc45)cc3)c3cccc4sc5ccccc5c34)cc12 ; c1ccc(-c2ccc(N(c3ccc(-c4cc5ccccc5c5ccccc45)cc3)c3ccc4ccc5ccccc5c4c3)c3oc4ccccc4c23)cc1 ; c1ccc(-c2ccc3oc4c(N(c5ccc(-c6cc7ccccc7c7ccccc67)cc5)c5ccc6ccc7ccccc7c6c5)cccc4c3c2)cc1] 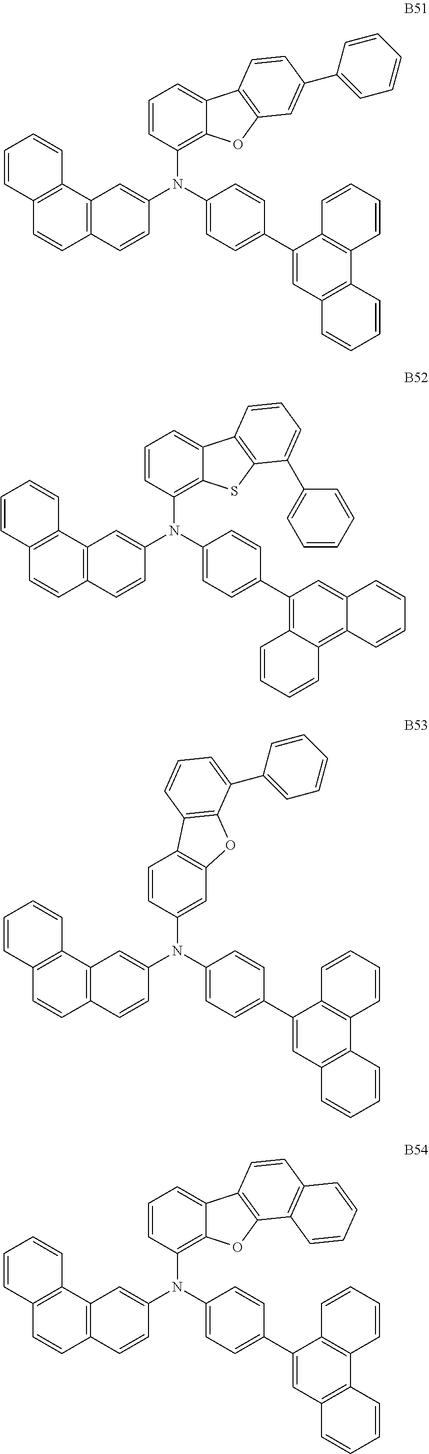 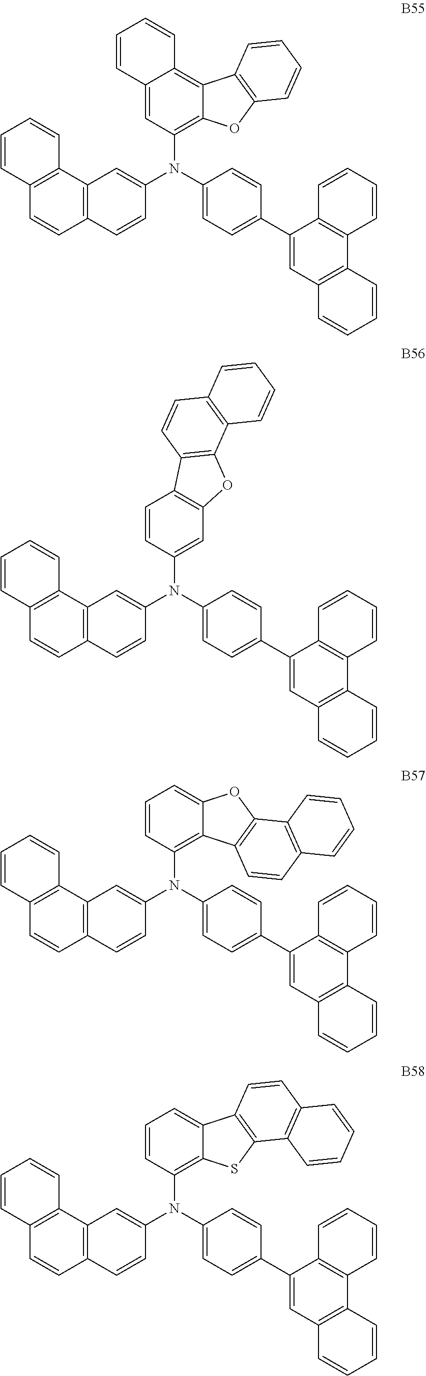 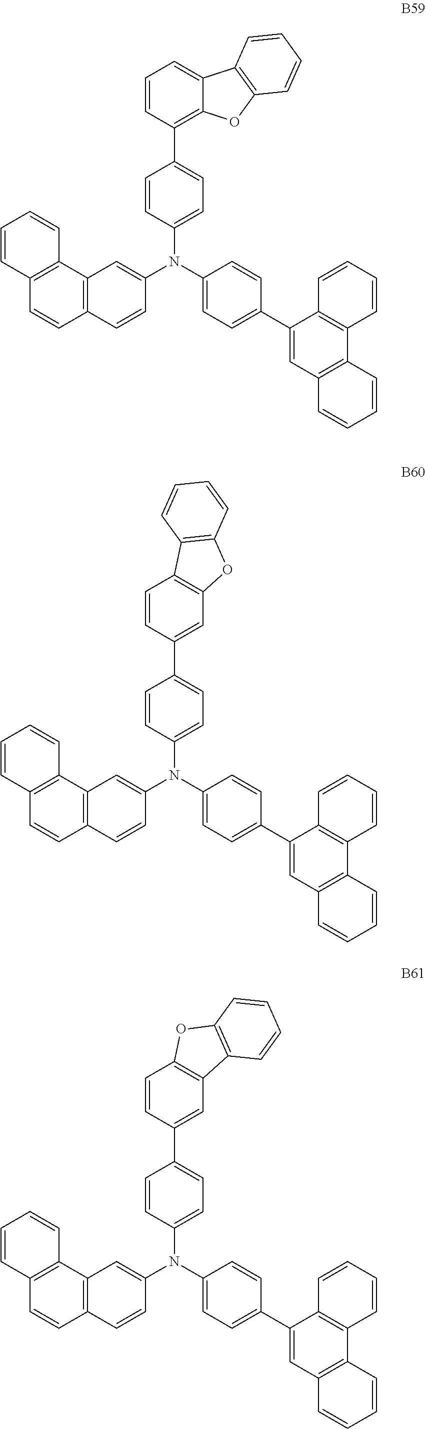 [SMILES: c1ccc2c(c1)ccc1ccc(N(c3ccc(-c4cc5ccccc5c5ccccc45)cc3)c3ccc(-c4cccc5c4oc4ccccc45)cc3)cc12 ; c1ccc2c(c1)ccc1ccc(N(c3ccc(-c4ccc5c(c4)oc4ccccc45)cc3)c3ccc(-c4cc5ccccc5c5ccccc45)cc3)cc12 ; c1ccc2c(c1)ccc1ccc(N(c3ccc(-c4ccc5oc6ccccc6c5c4)cc3)c3ccc(-c4cc5ccccc5c5ccccc45)cc3)cc12] 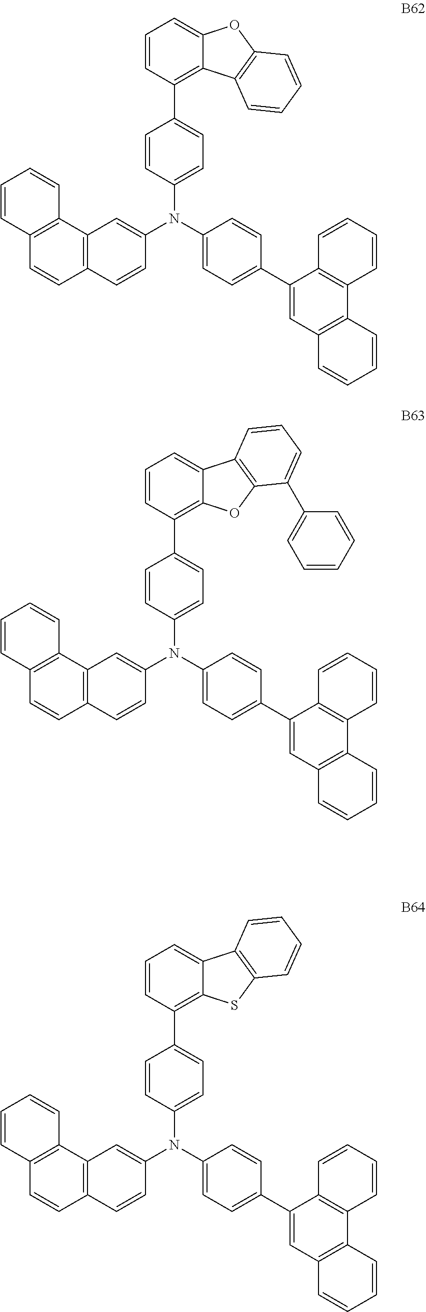 [SMILES: c1ccc2c(c1)ccc1ccc(N(c3ccc(-c4cc5ccccc5c5ccccc45)cc3)c3ccc(-c4cccc5oc6ccccc6c45)cc3)cc12 ; c1ccc(-c2cccc3c2oc2c(-c4ccc(N(c5ccc(-c6cc7ccccc7c7ccccc67)cc5)c5ccc6ccc7ccccc7c6c5)cc4)cccc23)cc1 ; c1ccc2c(c1)ccc1ccc(N(c3ccc(-c4cc5ccccc5c5ccccc45)cc3)c3ccc(-c4cccc5c4sc4ccccc45)cc3)cc12] 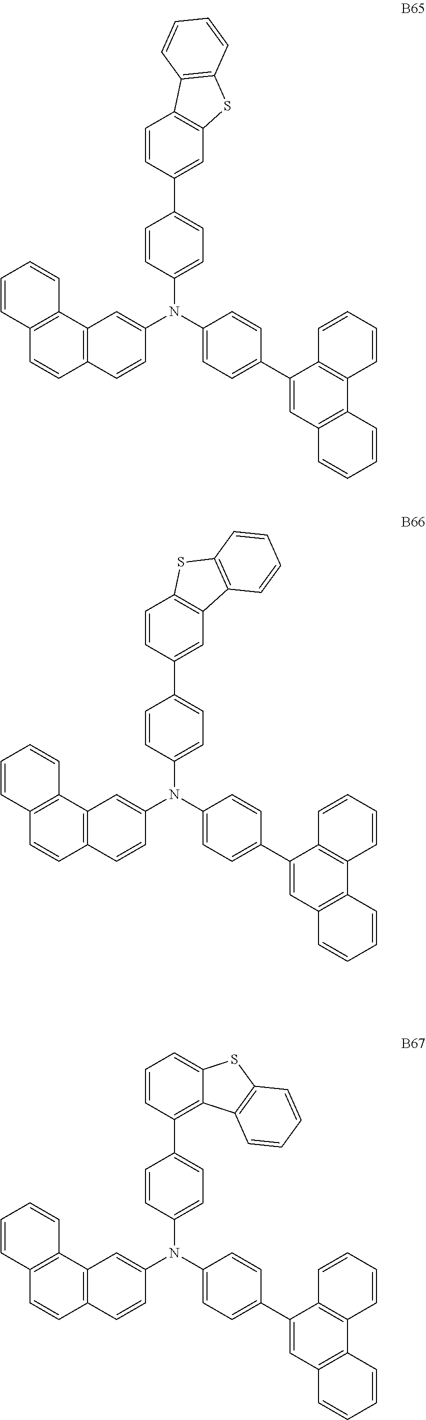 [SMILES: c1ccc2c(c1)ccc1ccc(N(c3ccc(-c4ccc5c(c4)sc4ccccc45)cc3)c3ccc(-c4cc5ccccc5c5ccccc45)cc3)cc12 ; c1ccc2c(c1)ccc1ccc(N(c3ccc(-c4ccc5sc6ccccc6c5c4)cc3)c3ccc(-c4cc5ccccc5c5ccccc45)cc3)cc12 ; c1ccc2c(c1)ccc1ccc(N(c3ccc(-c4cc5ccccc5c5ccccc45)cc3)c3ccc(-c4cccc5sc6ccccc6c45)cc3)cc12] 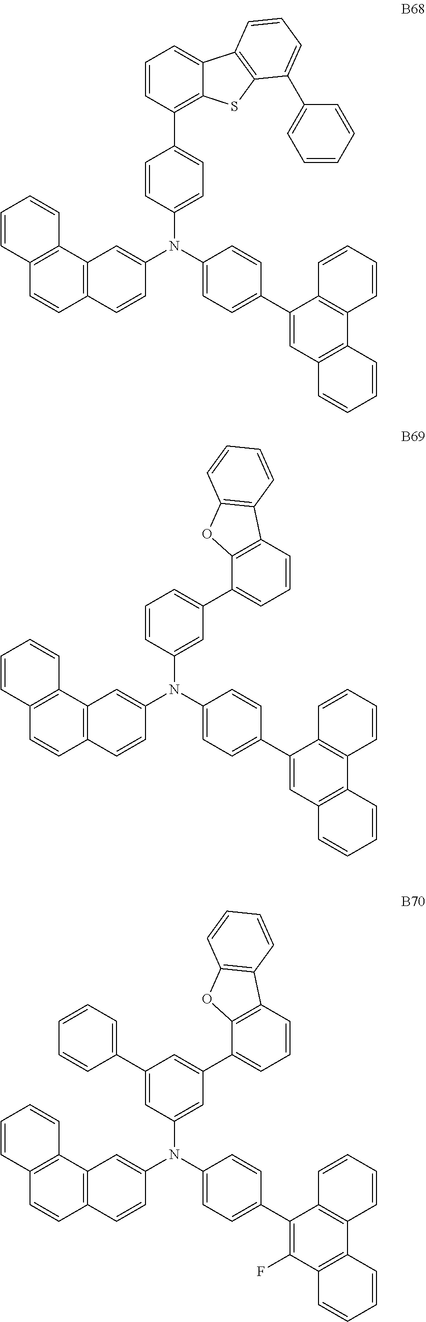 [SMILES: c1ccc(-c2cccc3c2sc2c(-c4ccc(N(c5ccc(-c6cc7ccccc7c7ccccc67)cc5)c5ccc6ccc7ccccc7c6c5)cc4)cccc23)cc1 ; c1cc(-c2cccc3c2oc2ccccc23)cc(N(c2ccc(-c3cc4ccccc4c4ccccc34)cc2)c2ccc3ccc4ccccc4c3c2)c1 ; Fc1c(-c2ccc(N(c3cc(-c4ccccc4)cc(-c4cccc5c4oc4ccccc45)c3)c3ccc4ccc5ccccc5c4c3)cc2)c2ccccc2c2ccccc12] 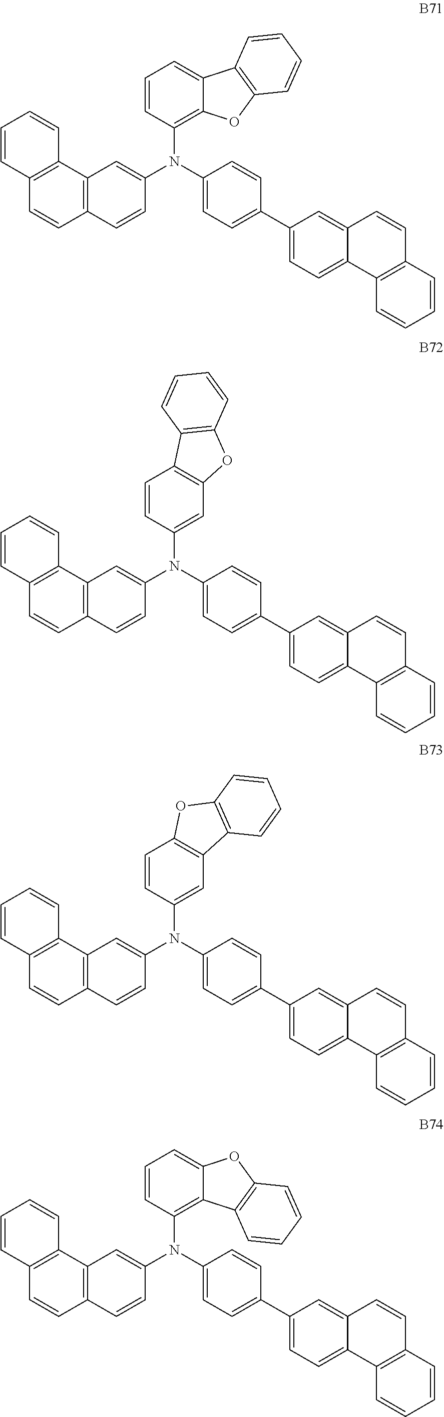 [SMILES: c1ccc2c(c1)ccc1cc(-c3ccc(N(c4ccc5ccc6ccccc6c5c4)c4cccc5c4oc4ccccc45)cc3)ccc12 ; c1ccc2c(c1)ccc1cc(-c3ccc(N(c4ccc5c(c4)oc4ccccc45)c4ccc5ccc6ccccc6c5c4)cc3)ccc12 ; c1ccc2c(c1)ccc1cc(-c3ccc(N(c4ccc5ccc6ccccc6c5c4)c4ccc5oc6ccccc6c5c4)cc3)ccc12 ; c1ccc2c(c1)ccc1cc(-c3ccc(N(c4ccc5ccc6ccccc6c5c4)c4cccc5oc6ccccc6c45)cc3)ccc12] 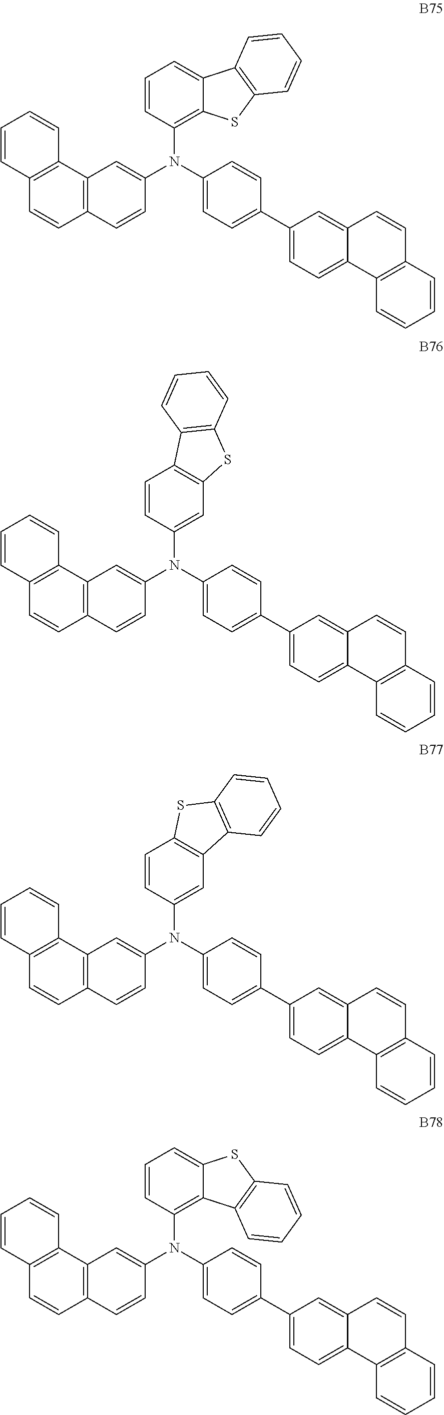 [SMILES: c1ccc2c(c1)ccc1cc(-c3ccc(N(c4ccc5ccc6ccccc6c5c4)c4cccc5c4sc4ccccc45)cc3)ccc12 ; c1ccc2c(c1)ccc1cc(-c3ccc(N(c4ccc5c(c4)sc4ccccc45)c4ccc5ccc6ccccc6c5c4)cc3)ccc12 ; c1ccc2c(c1)ccc1cc(-c3ccc(N(c4ccc5ccc6ccccc6c5c4)c4ccc5sc6ccccc6c5c4)cc3)ccc12 ; c1ccc2c(c1)ccc1cc(-c3ccc(N(c4ccc5ccc6ccccc6c5c4)c4cccc5sc6ccccc6c45)cc3)ccc12] 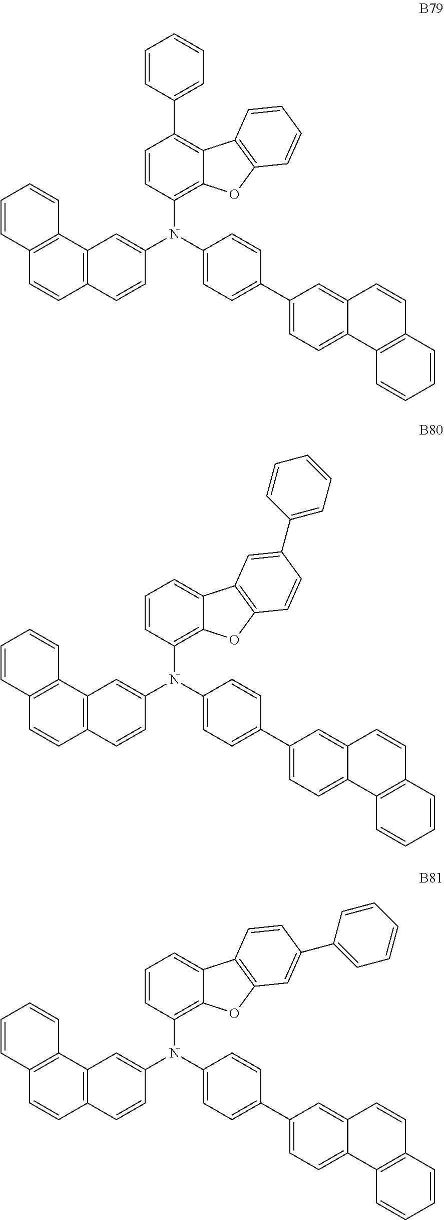 [SMILES: c1ccc(-c2ccc(N(c3ccc(-c4ccc5c(ccc6ccccc65)c4)cc3)c3ccc4ccc5ccccc5c4c3)c3oc4ccccc4c23)cc1 ; c1ccc(-c2ccc3oc4c(N(c5ccc(-c6ccc7c(ccc8ccccc87)c6)cc5)c5ccc6ccc7ccccc7c6c5)cccc4c3c2)cc1 ; c1ccc(-c2ccc3c(c2)oc2c(N(c4ccc(-c5ccc6c(ccc7ccccc76)c5)cc4)c4ccc5ccc6ccccc6c5c4)cccc23)cc1] 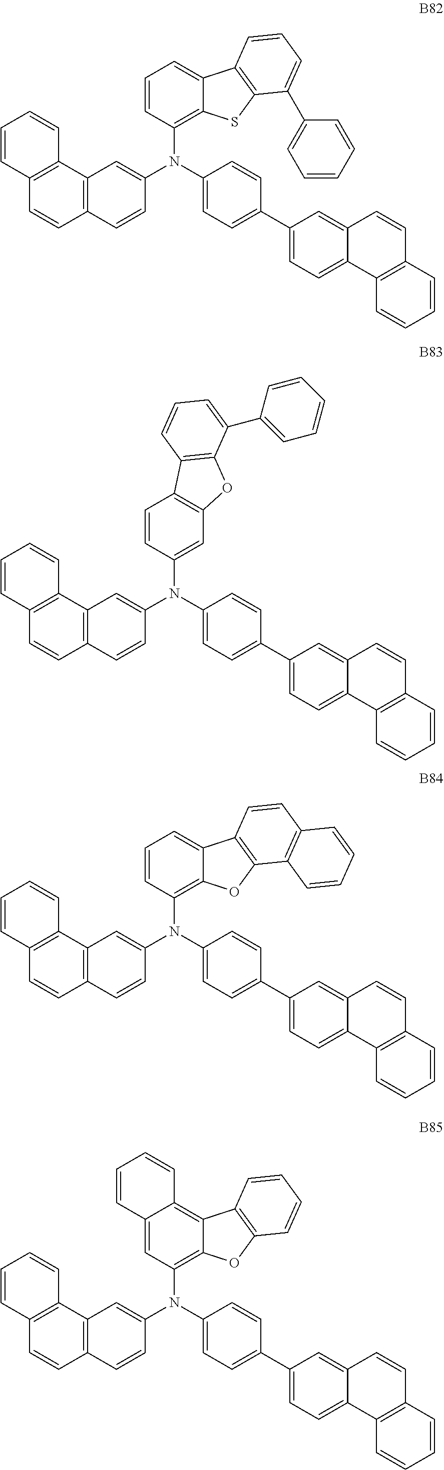 [SMILES: c1ccc(-c2cccc3c2sc2c(N(c4ccc(-c5ccc6c(ccc7ccccc76)c5)cc4)c4ccc5ccc6ccccc6c5c4)cccc23)cc1 ; c1ccc(-c2cccc3c2oc2cc(N(c4ccc(-c5ccc6c(ccc7ccccc76)c5)cc4)c4ccc5ccc6ccccc6c5c4)ccc23)cc1 ; c1ccc2c(c1)ccc1cc(-c3ccc(N(c4ccc5ccc6ccccc6c5c4)c4cccc5c4oc4c6ccccc6ccc54)cc3)ccc12 ; c1ccc2c(c1)ccc1cc(-c3ccc(N(c4ccc5ccc6ccccc6c5c4)c4cc5ccccc5c5c4oc4ccccc45)cc3)ccc12] 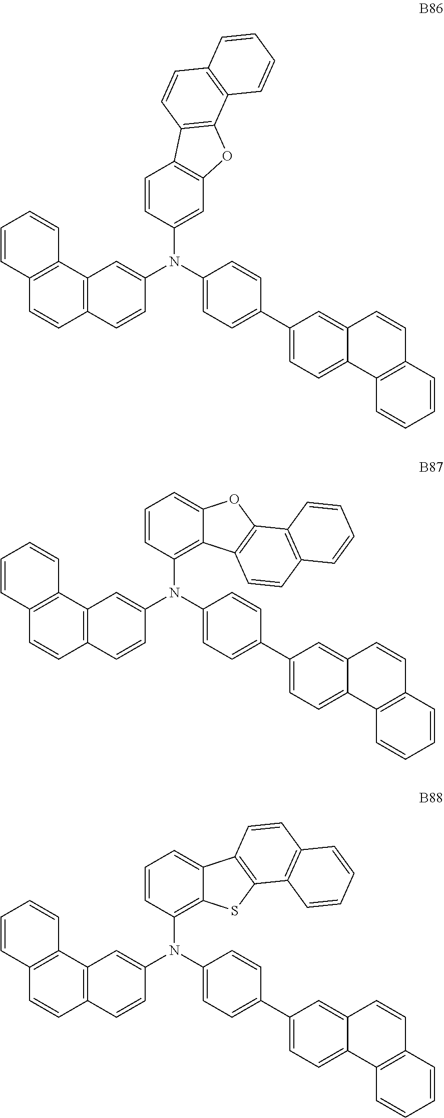 [SMILES: c1ccc2c(c1)ccc1cc(-c3ccc(N(c4ccc5c(c4)oc4c6ccccc6ccc54)c4ccc5ccc6ccccc6c5c4)cc3)ccc12 ; c1ccc2c(c1)ccc1cc(-c3ccc(N(c4ccc5ccc6ccccc6c5c4)c4cccc5oc6c7ccccc7ccc6c45)cc3)ccc12 ; c1ccc2c(c1)ccc1cc(-c3ccc(N(c4ccc5ccc6ccccc6c5c4)c4cccc5c4sc4c6ccccc6ccc54)cc3)ccc12] 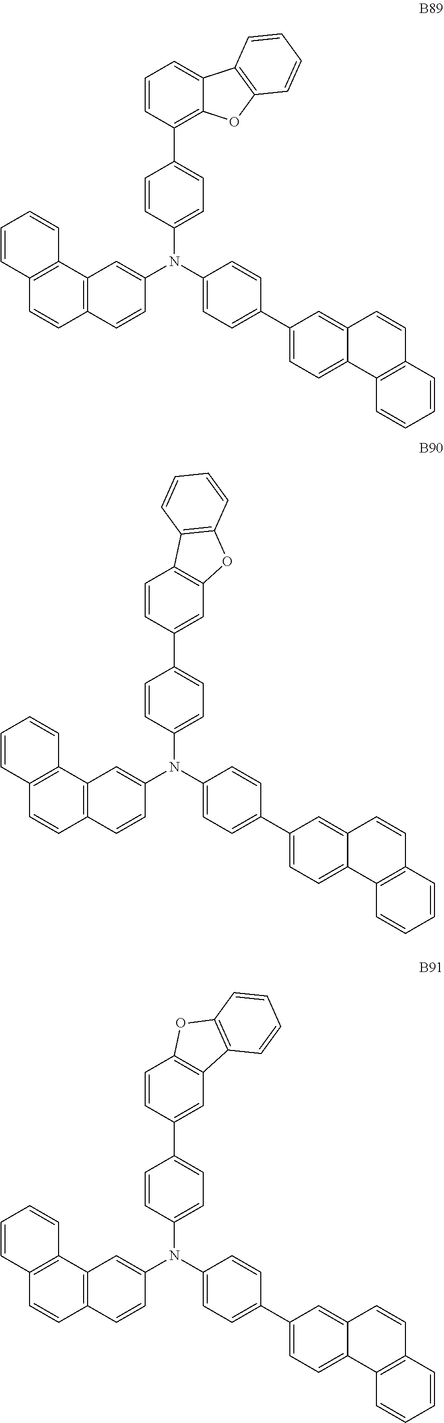 [SMILES: c1ccc2c(c1)ccc1cc(-c3ccc(N(c4ccc(-c5cccc6c5oc5ccccc56)cc4)c4ccc5ccc6ccccc6c5c4)cc3)ccc12 ; c1ccc2c(c1)ccc1cc(-c3ccc(N(c4ccc(-c5ccc6c(c5)oc5ccccc56)cc4)c4ccc5ccc6ccccc6c5c4)cc3)ccc12 ; c1ccc2c(c1)ccc1cc(-c3ccc(N(c4ccc(-c5ccc6oc7ccccc7c6c5)cc4)c4ccc5ccc6ccccc6c5c4)cc3)ccc12] 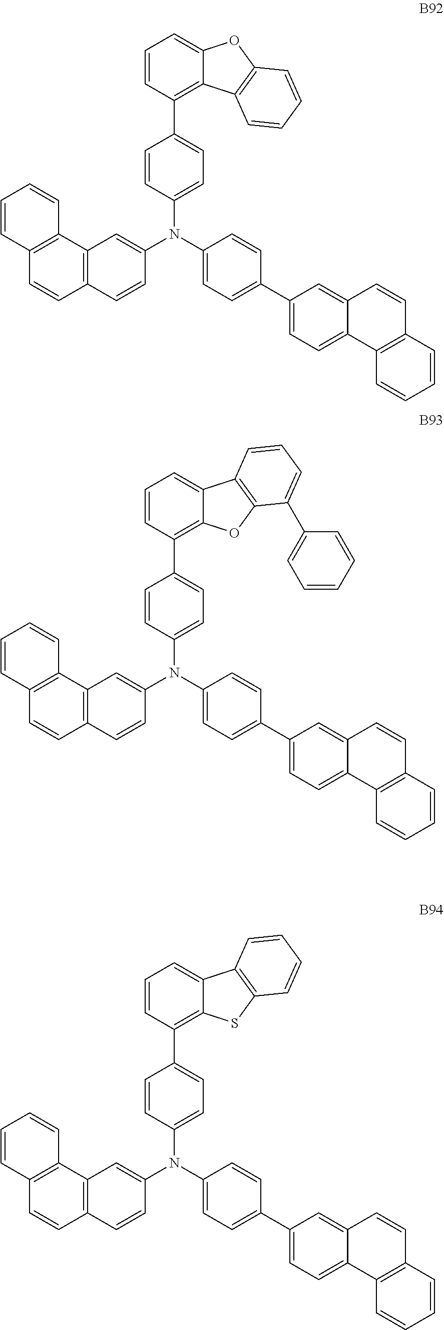 [SMILES: c1ccc2c(c1)ccc1cc(-c3ccc(N(c4ccc(-c5cccc6oc7ccccc7c56)cc4)c4ccc5ccc6ccccc6c5c4)cc3)ccc12 ; c1ccc(-c2cccc3c2oc2c(-c4ccc(N(c5ccc(-c6ccc7c(ccc8ccccc87)c6)cc5)c5ccc6ccc7ccccc7c6c5)cc4)cccc23)cc1 ; c1ccc2c(c1)ccc1cc(-c3ccc(N(c4ccc(-c5cccc6c5sc5ccccc56)cc4)c4ccc5ccc6ccccc6c5c4)cc3)ccc12] 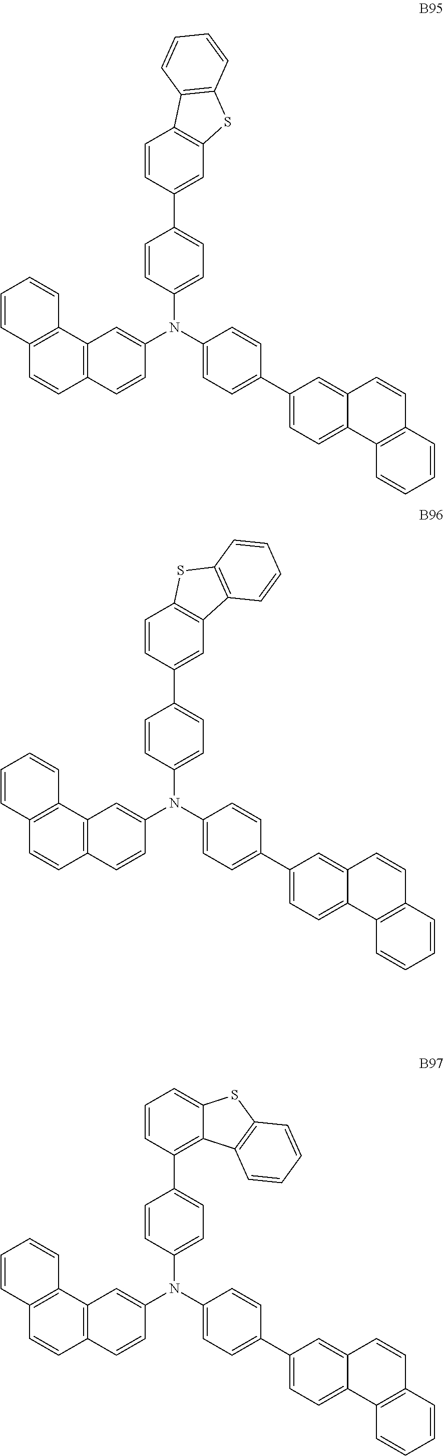 [SMILES: c1ccc2c(c1)ccc1cc(-c3ccc(N(c4ccc(-c5ccc6c(c5)sc5ccccc56)cc4)c4ccc5ccc6ccccc6c5c4)cc3)ccc12 ; c1ccc2c(c1)ccc1cc(-c3ccc(N(c4ccc(-c5ccc6sc7ccccc7c6c5)cc4)c4ccc5ccc6ccccc6c5c4)cc3)ccc12 ; c1ccc2c(c1)ccc1cc(-c3ccc(N(c4ccc(-c5cccc6sc7ccccc7c56)cc4)c4ccc5ccc6ccccc6c5c4)cc3)ccc12] 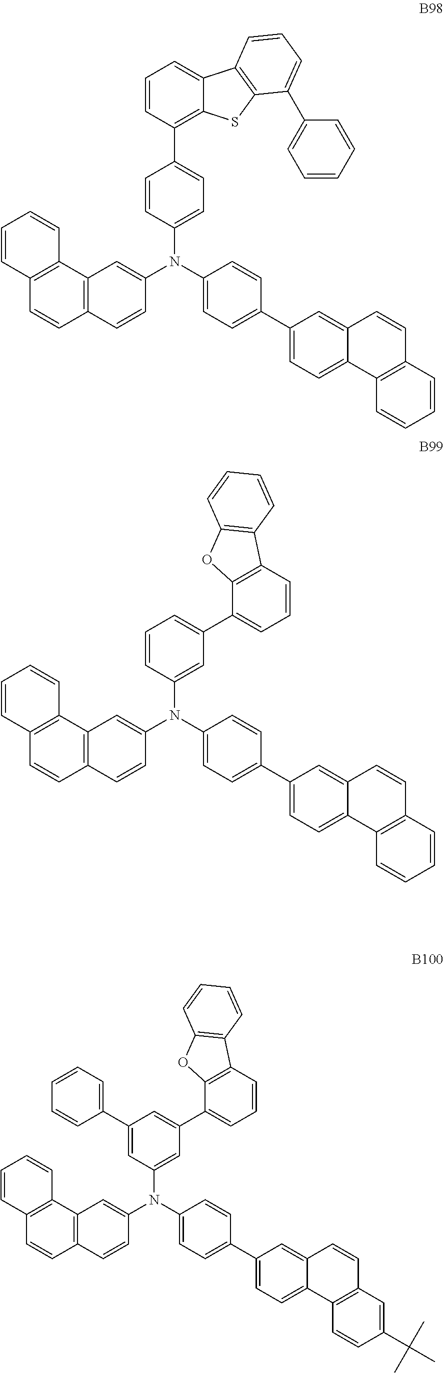 [SMILES: c1ccc(-c2cccc3c2sc2c(-c4ccc(N(c5ccc(-c6ccc7c(ccc8ccccc87)c6)cc5)c5ccc6ccc7ccccc7c6c5)cc4)cccc23)cc1 ; c1cc(-c2cccc3c2oc2ccccc23)cc(N(c2ccc(-c3ccc4c(ccc5ccccc54)c3)cc2)c2ccc3ccc4ccccc4c3c2)c1 ; CC(C)(C)c1ccc2c(ccc3cc(-c4ccc(N(c5cc(-c6ccccc6)cc(-c6cccc7c6oc6ccccc67)c5)c5ccc6ccc7ccccc7c6c5)cc4)ccc32)c1] 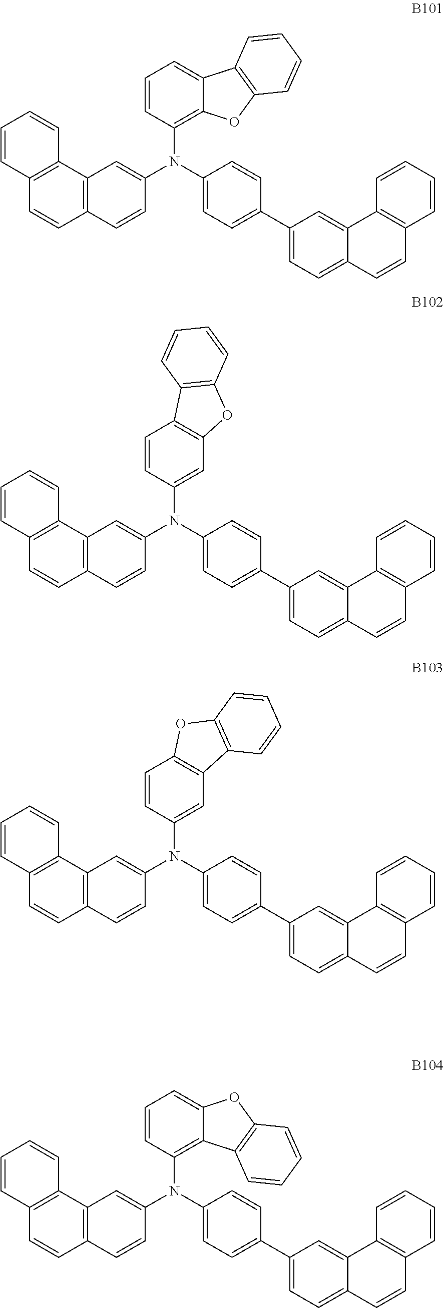 [SMILES: c1ccc2c(c1)ccc1ccc(-c3ccc(N(c4ccc5ccc6ccccc6c5c4)c4cccc5c4oc4ccccc45)cc3)cc12 ; c1ccc2c(c1)ccc1ccc(-c3ccc(N(c4ccc5c(c4)oc4ccccc45)c4ccc5ccc6ccccc6c5c4)cc3)cc12 ; c1ccc2c(c1)ccc1ccc(-c3ccc(N(c4ccc5ccc6ccccc6c5c4)c4ccc5oc6ccccc6c5c4)cc3)cc12 ; c1ccc2c(c1)ccc1ccc(-c3ccc(N(c4ccc5ccc6ccccc6c5c4)c4cccc5oc6ccccc6c45)cc3)cc12] 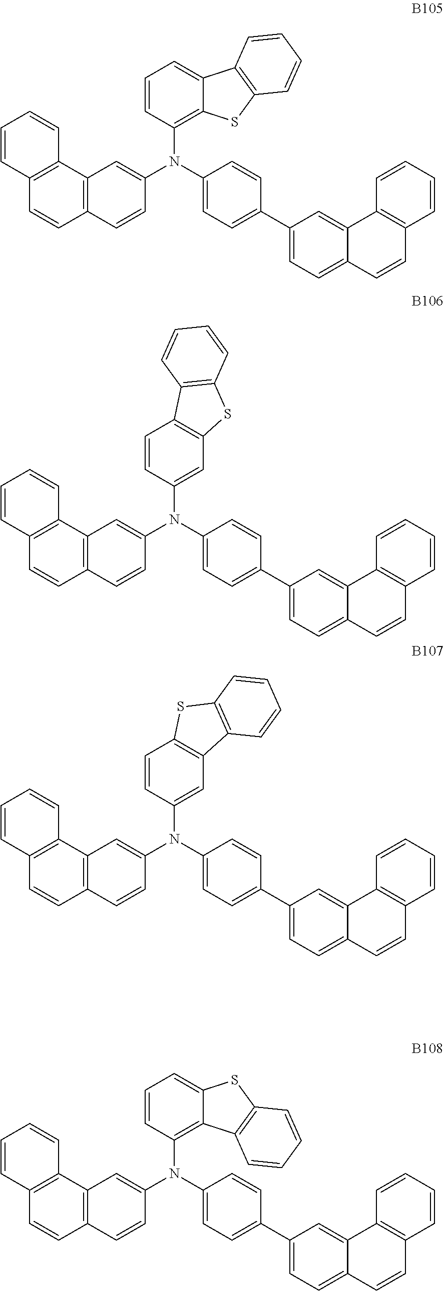 [SMILES: c1ccc2c(c1)ccc1ccc(-c3ccc(N(c4ccc5ccc6ccccc6c5c4)c4cccc5c4sc4ccccc45)cc3)cc12 ; c1ccc2c(c1)ccc1ccc(-c3ccc(N(c4ccc5c(c4)sc4ccccc45)c4ccc5ccc6ccccc6c5c4)cc3)cc12 ; c1ccc2c(c1)ccc1ccc(-c3ccc(N(c4ccc5ccc6ccccc6c5c4)c4ccc5sc6ccccc6c5c4)cc3)cc12 ; c1ccc2c(c1)ccc1ccc(-c3ccc(N(c4ccc5ccc6ccccc6c5c4)c4cccc5sc6ccccc6c45)cc3)cc12] 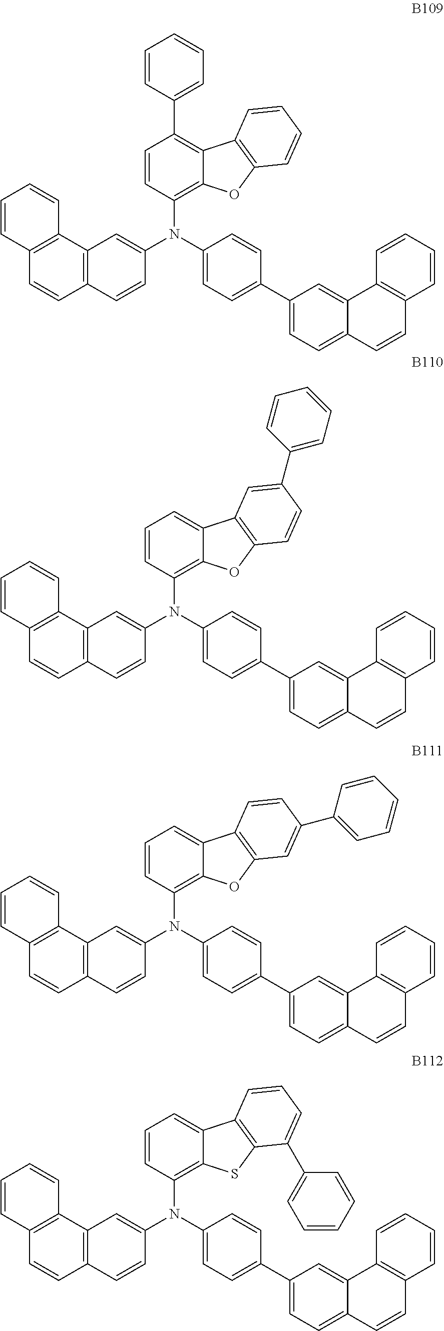 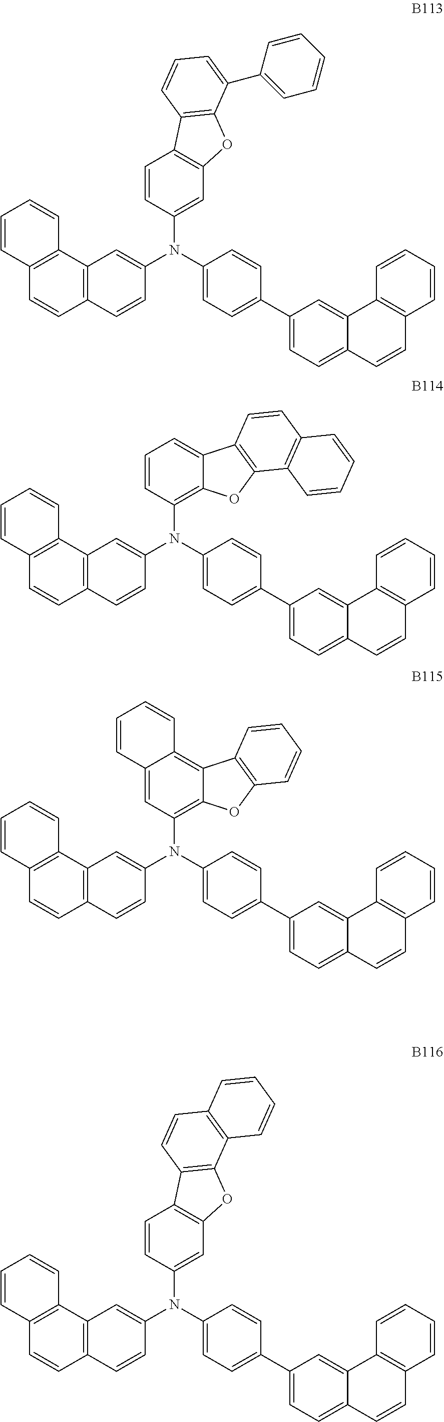 [SMILES: c1ccc(-c2cccc3c2oc2cc(N(c4ccc(-c5ccc6ccc7ccccc7c6c5)cc4)c4ccc5ccc6ccccc6c5c4)ccc23)cc1 ; c1ccc2c(c1)ccc1ccc(-c3ccc(N(c4ccc5ccc6ccccc6c5c4)c4cccc5c4oc4c6ccccc6ccc54)cc3)cc12 ; c1ccc2c(c1)ccc1ccc(-c3ccc(N(c4ccc5ccc6ccccc6c5c4)c4cc5ccccc5c5c4oc4ccccc45)cc3)cc12 ; c1ccc2c(c1)ccc1ccc(-c3ccc(N(c4ccc5c(c4)oc4c6ccccc6ccc54)c4ccc5ccc6ccccc6c5c4)cc3)cc12] 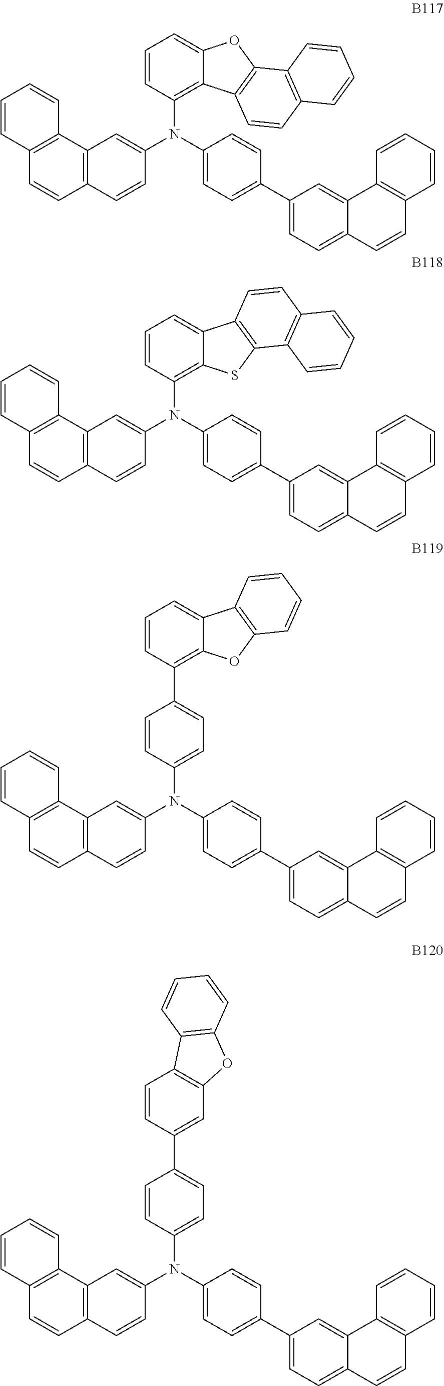 [SMILES: c1ccc2c(c1)ccc1ccc(-c3ccc(N(c4ccc5ccc6ccccc6c5c4)c4cccc5oc6c7ccccc7ccc6c45)cc3)cc12 ; c1ccc2c(c1)ccc1ccc(-c3ccc(N(c4ccc5ccc6ccccc6c5c4)c4cccc5c4sc4c6ccccc6ccc54)cc3)cc12 ; c1ccc2c(c1)ccc1ccc(-c3ccc(N(c4ccc(-c5cccc6c5oc5ccccc56)cc4)c4ccc5ccc6ccccc6c5c4)cc3)cc12 ; c1ccc2c(c1)ccc1ccc(-c3ccc(N(c4ccc(-c5ccc6c(c5)oc5ccccc56)cc4)c4ccc5ccc6ccccc6c5c4)cc3)cc12] 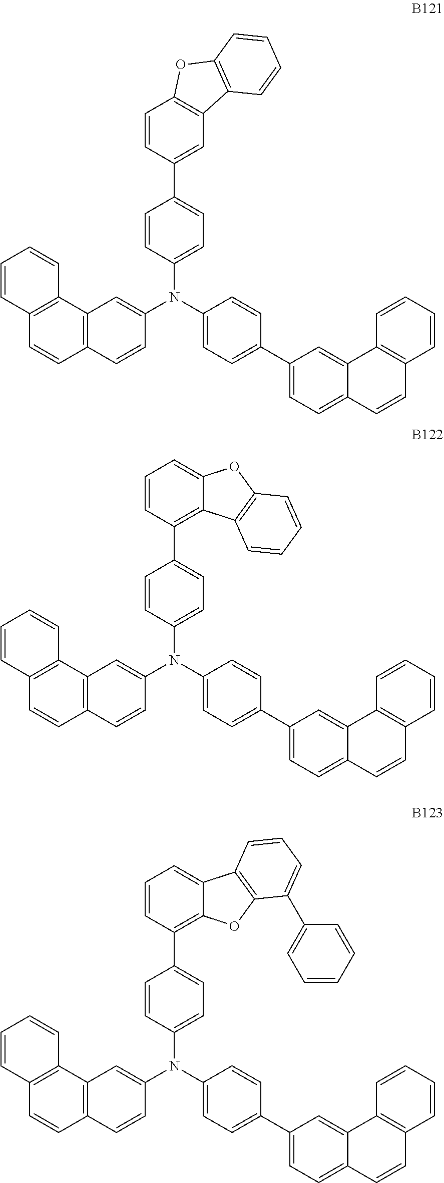 [SMILES: c1ccc2c(c1)ccc1ccc(-c3ccc(N(c4ccc(-c5ccc6oc7ccccc7c6c5)cc4)c4ccc5ccc6ccccc6c5c4)cc3)cc12 ; c1ccc2c(c1)ccc1ccc(-c3ccc(N(c4ccc(-c5cccc6oc7ccccc7c56)cc4)c4ccc5ccc6ccccc6c5c4)cc3)cc12 ; c1ccc(-c2cccc3c2oc2c(-c4ccc(N(c5ccc(-c6ccc7ccc8ccccc8c7c6)cc5)c5ccc6ccc7ccccc7c6c5)cc4)cccc23)cc1] 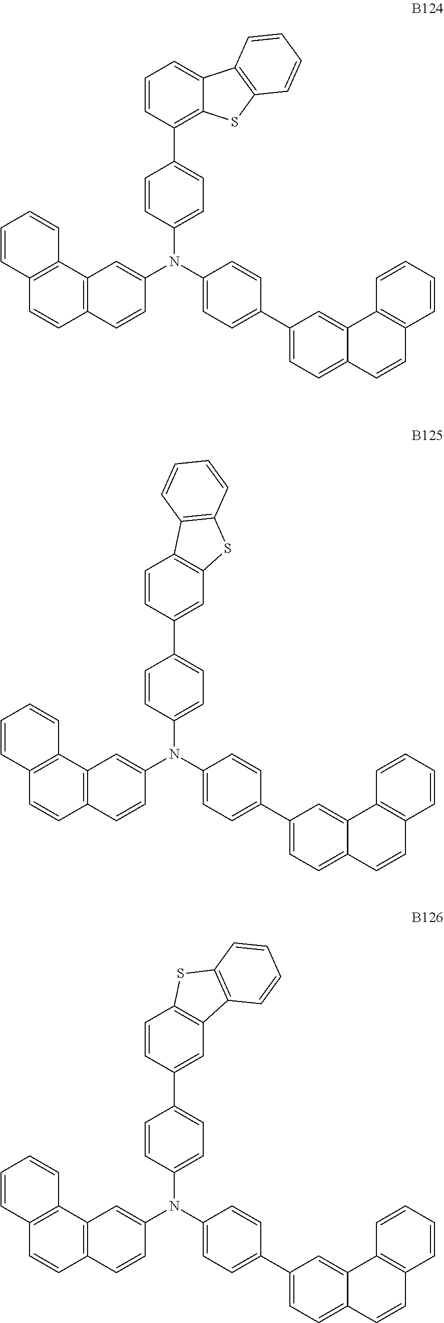 [SMILES: c1ccc2c(c1)ccc1ccc(-c3ccc(N(c4ccc(-c5cccc6c5sc5ccccc56)cc4)c4ccc5ccc6ccccc6c5c4)cc3)cc12 ; c1ccc2c(c1)ccc1ccc(-c3ccc(N(c4ccc(-c5ccc6c(c5)sc5ccccc56)cc4)c4ccc5ccc6ccccc6c5c4)cc3)cc12 ; c1ccc2c(c1)ccc1ccc(-c3ccc(N(c4ccc(-c5ccc6sc7ccccc7c6c5)cc4)c4ccc5ccc6ccccc6c5c4)cc3)cc12] 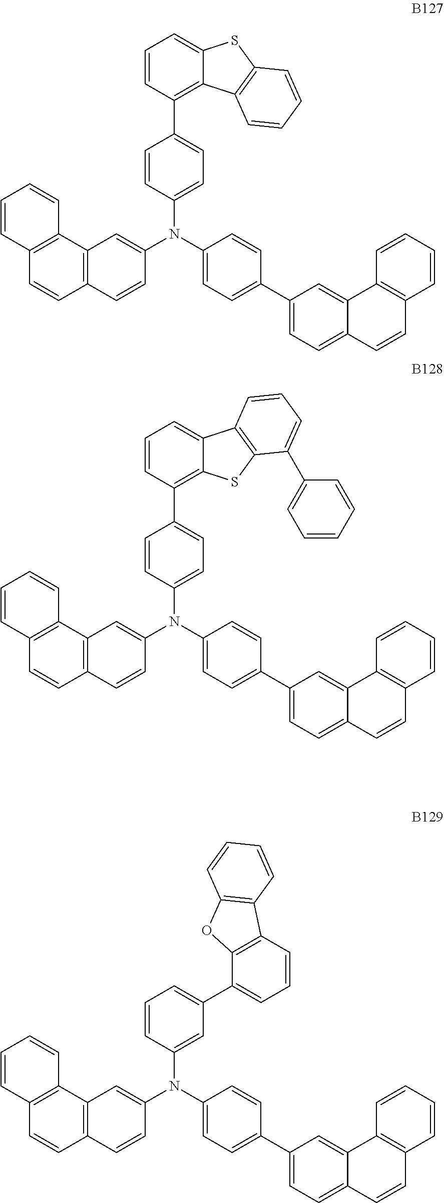 [SMILES: c1ccc2c(c1)ccc1ccc(-c3ccc(N(c4ccc(-c5cccc6sc7ccccc7c56)cc4)c4ccc5ccc6ccccc6c5c4)cc3)cc12 ; c1ccc(-c2cccc3c2sc2c(-c4ccc(N(c5ccc(-c6ccc7ccc8ccccc8c7c6)cc5)c5ccc6ccc7ccccc7c6c5)cc4)cccc23)cc1 ; c1cc(-c2cccc3c2oc2ccccc23)cc(N(c2ccc(-c3ccc4ccc5ccccc5c4c3)cc2)c2ccc3ccc4ccccc4c3c2)c1] 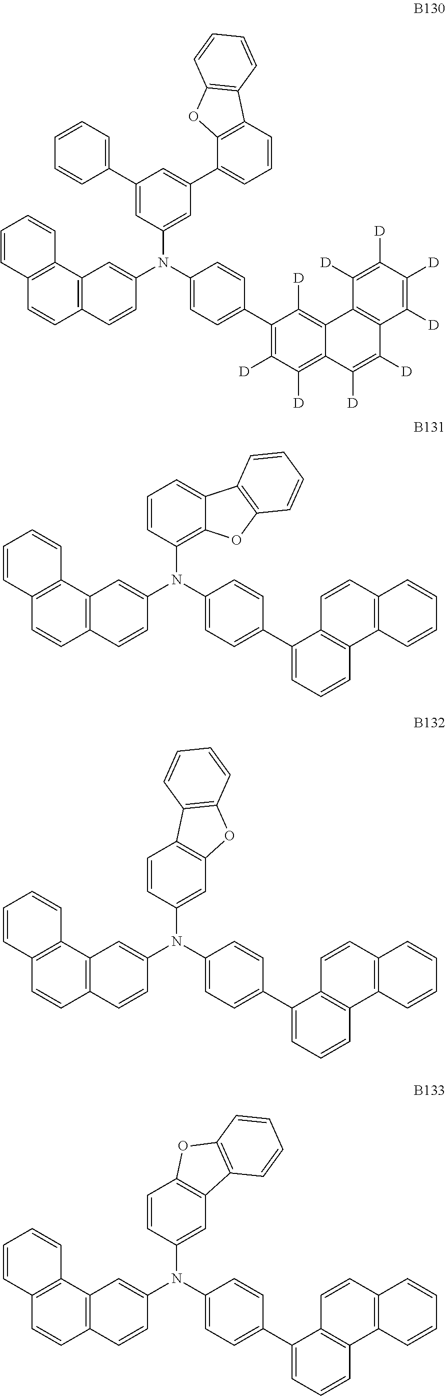 [SMILES: [2H]c1c([2H])c([2H])c2c(c1[2H])c([2H])c([2H])c1c([2H])c([2H])c(-c3ccc(N(c4cc(-c5ccccc5)cc(-c5cccc6c5oc5ccccc56)c4)c4ccc5ccc6ccccc6c5c4)cc3)c([2H])c12 ; c1ccc2c(c1)ccc1ccc(N(c3ccc(-c4cccc5c4ccc4ccccc45)cc3)c3cccc4c3oc3ccccc34)cc12 ; c1ccc2c(c1)ccc1ccc(N(c3ccc(-c4cccc5c4ccc4ccccc45)cc3)c3ccc4c(c3)oc3ccccc34)cc12 ; c1ccc2c(c1)ccc1ccc(N(c3ccc(-c4cccc5c4ccc4ccccc45)cc3)c3ccc4oc5ccccc5c4c3)cc12] 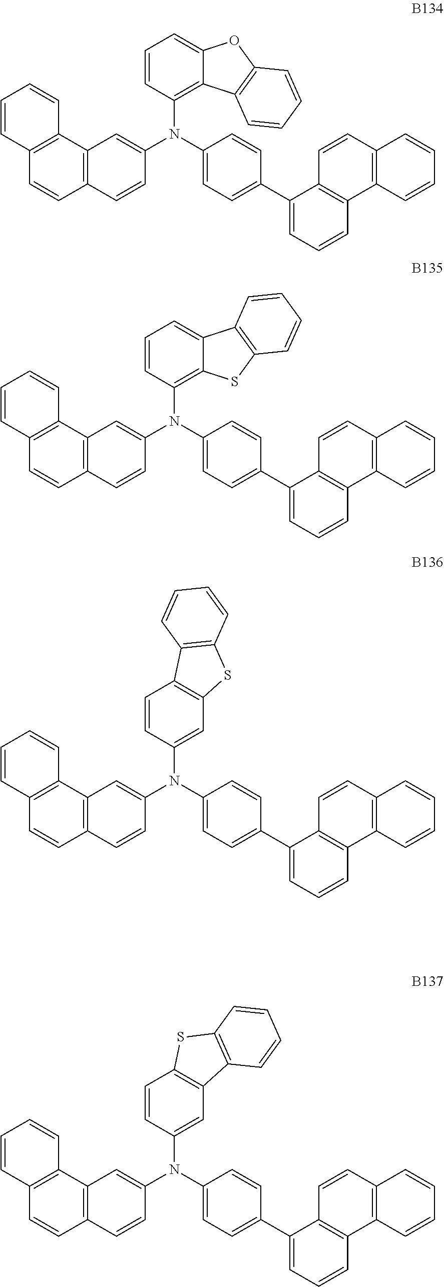 [SMILES: c1ccc2c(c1)ccc1ccc(N(c3ccc(-c4cccc5c4ccc4ccccc45)cc3)c3cccc4oc5ccccc5c34)cc12 ; c1ccc2c(c1)ccc1ccc(N(c3ccc(-c4cccc5c4ccc4ccccc45)cc3)c3cccc4c3sc3ccccc34)cc12 ; c1ccc2c(c1)ccc1ccc(N(c3ccc(-c4cccc5c4ccc4ccccc45)cc3)c3ccc4c(c3)sc3ccccc34)cc12 ; c1ccc2c(c1)ccc1ccc(N(c3ccc(-c4cccc5c4ccc4ccccc45)cc3)c3ccc4sc5ccccc5c4c3)cc12] 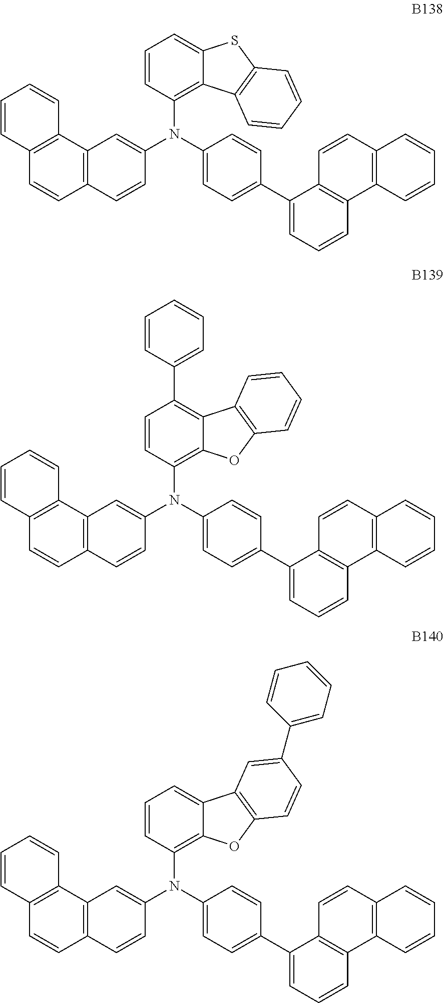 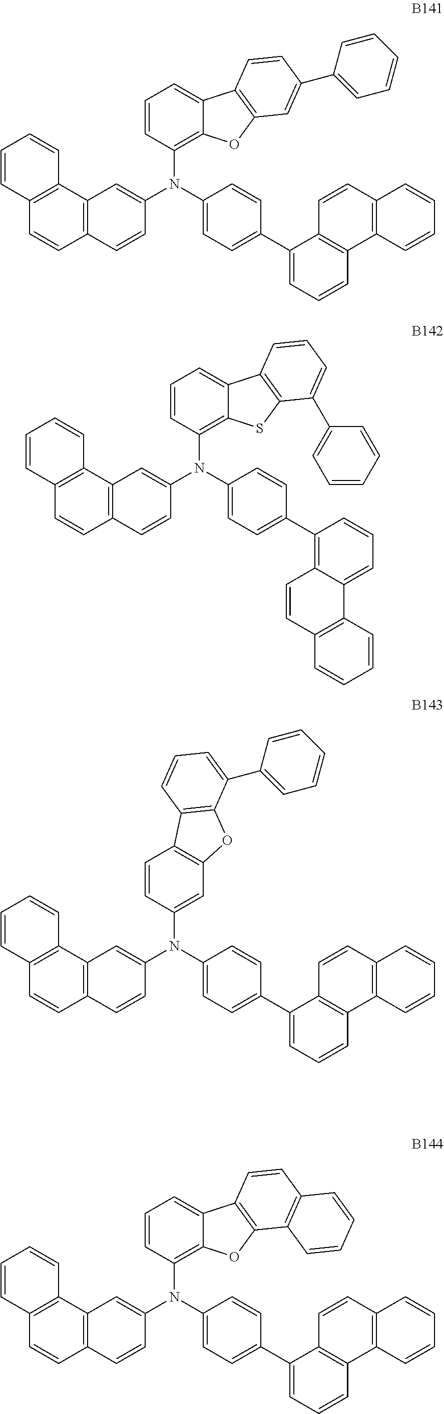 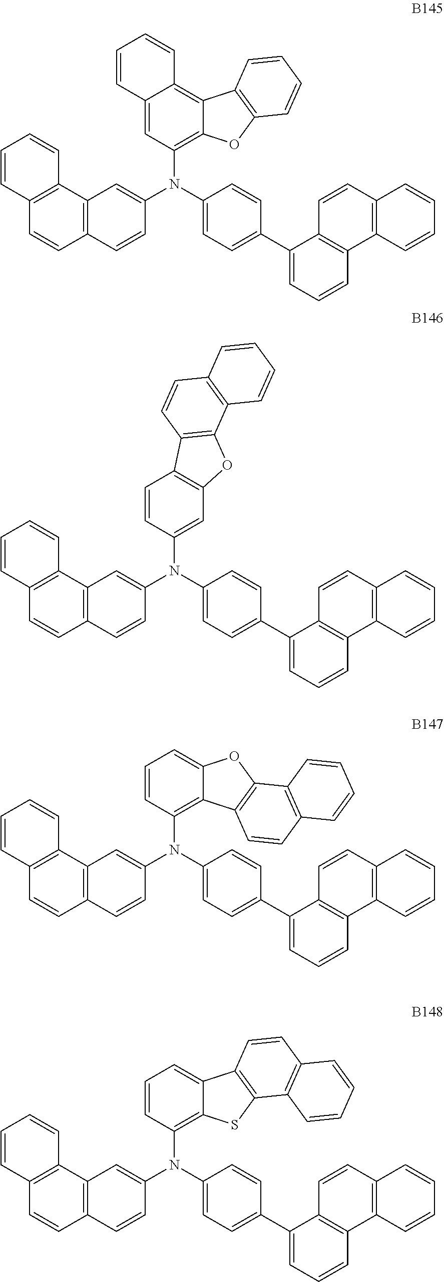 [SMILES: c1ccc2c(c1)ccc1ccc(N(c3ccc(-c4cccc5c4ccc4ccccc45)cc3)c3cc4ccccc4c4c3oc3ccccc34)cc12 ; c1ccc2c(c1)ccc1ccc(N(c3ccc(-c4cccc5c4ccc4ccccc45)cc3)c3ccc4c(c3)oc3c5ccccc5ccc43)cc12 ; c1ccc2c(c1)ccc1ccc(N(c3ccc(-c4cccc5c4ccc4ccccc45)cc3)c3cccc4oc5c6ccccc6ccc5c34)cc12 ; c1ccc2c(c1)ccc1ccc(N(c3ccc(-c4cccc5c4ccc4ccccc45)cc3)c3cccc4c3sc3c5ccccc5ccc43)cc12] 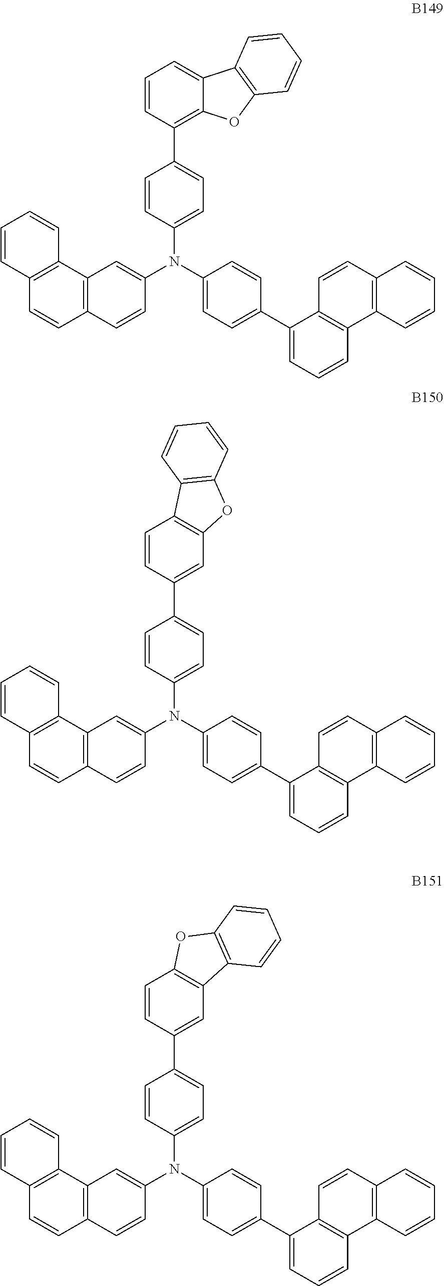 [SMILES: c1ccc2c(c1)ccc1ccc(N(c3ccc(-c4cccc5c4ccc4ccccc45)cc3)c3ccc(-c4cccc5c4oc4ccccc45)cc3)cc12 ; c1ccc2c(c1)ccc1ccc(N(c3ccc(-c4ccc5c(c4)oc4ccccc45)cc3)c3ccc(-c4cccc5c4ccc4ccccc45)cc3)cc12 ; c1ccc2c(c1)ccc1ccc(N(c3ccc(-c4ccc5oc6ccccc6c5c4)cc3)c3ccc(-c4cccc5c4ccc4ccccc45)cc3)cc12] 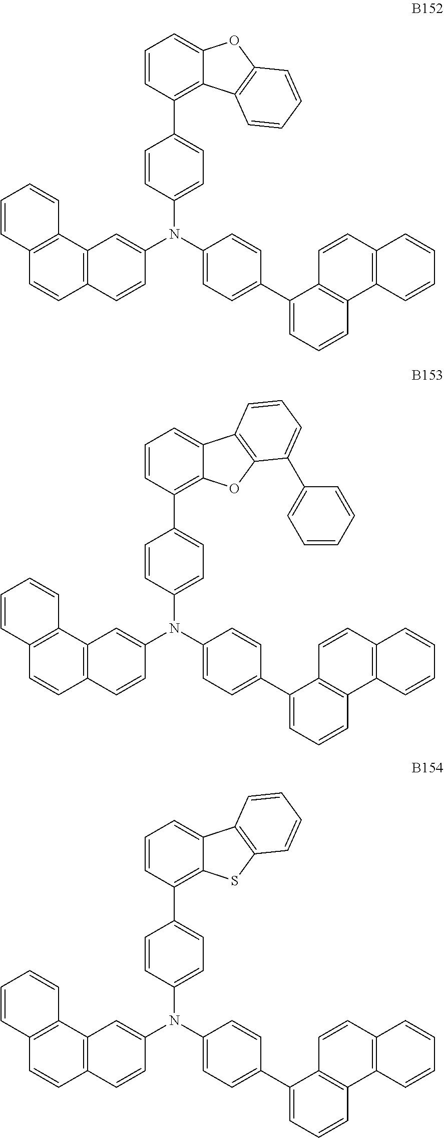 [SMILES: c1ccc2c(c1)ccc1ccc(N(c3ccc(-c4cccc5c4ccc4ccccc45)cc3)c3ccc(-c4cccc5oc6ccccc6c45)cc3)cc12 ; c1ccc(-c2cccc3c2oc2c(-c4ccc(N(c5ccc(-c6cccc7c6ccc6ccccc67)cc5)c5ccc6ccc7ccccc7c6c5)cc4)cccc23)cc1 ; c1ccc2c(c1)ccc1ccc(N(c3ccc(-c4cccc5c4ccc4ccccc45)cc3)c3ccc(-c4cccc5c4sc4ccccc45)cc3)cc12] 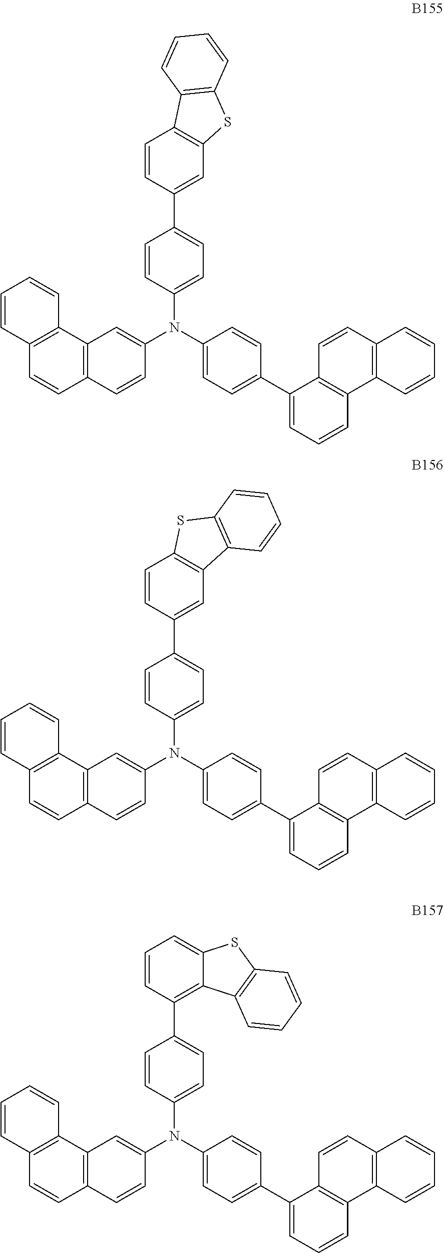 [SMILES: c1ccc2c(c1)ccc1ccc(N(c3ccc(-c4ccc5c(c4)sc4ccccc45)cc3)c3ccc(-c4cccc5c4ccc4ccccc45)cc3)cc12 ; c1ccc2c(c1)ccc1ccc(N(c3ccc(-c4ccc5sc6ccccc6c5c4)cc3)c3ccc(-c4cccc5c4ccc4ccccc45)cc3)cc12 ; c1ccc2c(c1)ccc1ccc(N(c3ccc(-c4cccc5c4ccc4ccccc45)cc3)c3ccc(-c4cccc5sc6ccccc6c45)cc3)cc12] 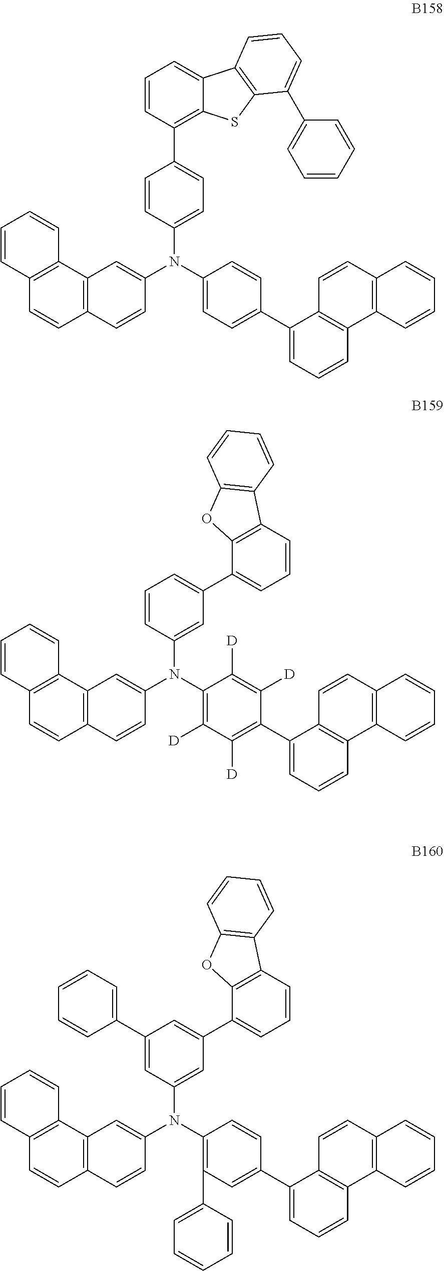 [SMILES: c1ccc(-c2cccc3c2sc2c(-c4ccc(N(c5ccc(-c6cccc7c6ccc6ccccc67)cc5)c5ccc6ccc7ccccc7c6c5)cc4)cccc23)cc1 ; [2H]c1c([2H])c(N(c2cccc(-c3cccc4c3oc3ccccc34)c2)c2ccc3ccc4ccccc4c3c2)c([2H])c([2H])c1-c1cccc2c1ccc1ccccc12 ; c1ccc(-c2cc(-c3cccc4c3oc3ccccc34)cc(N(c3ccc4ccc5ccccc5c4c3)c3ccc(-c4cccc5c4ccc4ccccc45)cc3-c3ccccc3)c2)cc1] 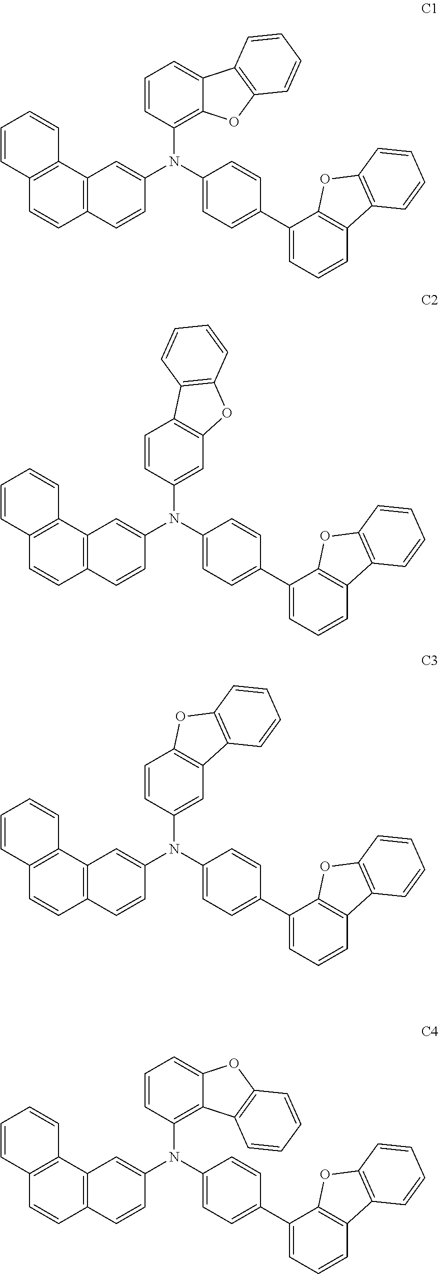 [SMILES: c1ccc2c(c1)ccc1ccc(N(c3ccc(-c4cccc5c4oc4ccccc45)cc3)c3cccc4c3oc3ccccc34)cc12 ; c1ccc2c(c1)ccc1ccc(N(c3ccc(-c4cccc5c4oc4ccccc45)cc3)c3ccc4c(c3)oc3ccccc34)cc12 ; c1ccc2c(c1)ccc1ccc(N(c3ccc(-c4cccc5c4oc4ccccc45)cc3)c3ccc4oc5ccccc5c4c3)cc12 ; c1ccc2c(c1)ccc1ccc(N(c3ccc(-c4cccc5c4oc4ccccc45)cc3)c3cccc4oc5ccccc5c34)cc12] 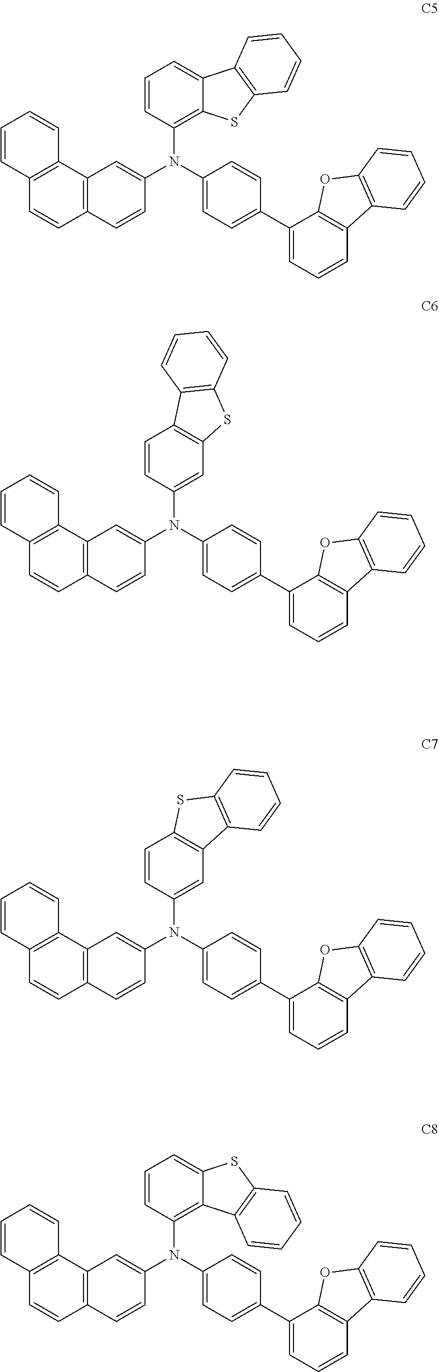 [SMILES: c1ccc2c(c1)ccc1ccc(N(c3ccc(-c4cccc5c4oc4ccccc45)cc3)c3cccc4c3sc3ccccc34)cc12 ; c1ccc2c(c1)ccc1ccc(N(c3ccc(-c4cccc5c4oc4ccccc45)cc3)c3ccc4c(c3)sc3ccccc34)cc12 ; c1ccc2c(c1)ccc1ccc(N(c3ccc(-c4cccc5c4oc4ccccc45)cc3)c3ccc4sc5ccccc5c4c3)cc12 ; c1ccc2c(c1)ccc1ccc(N(c3ccc(-c4cccc5c4oc4ccccc45)cc3)c3cccc4sc5ccccc5c34)cc12] 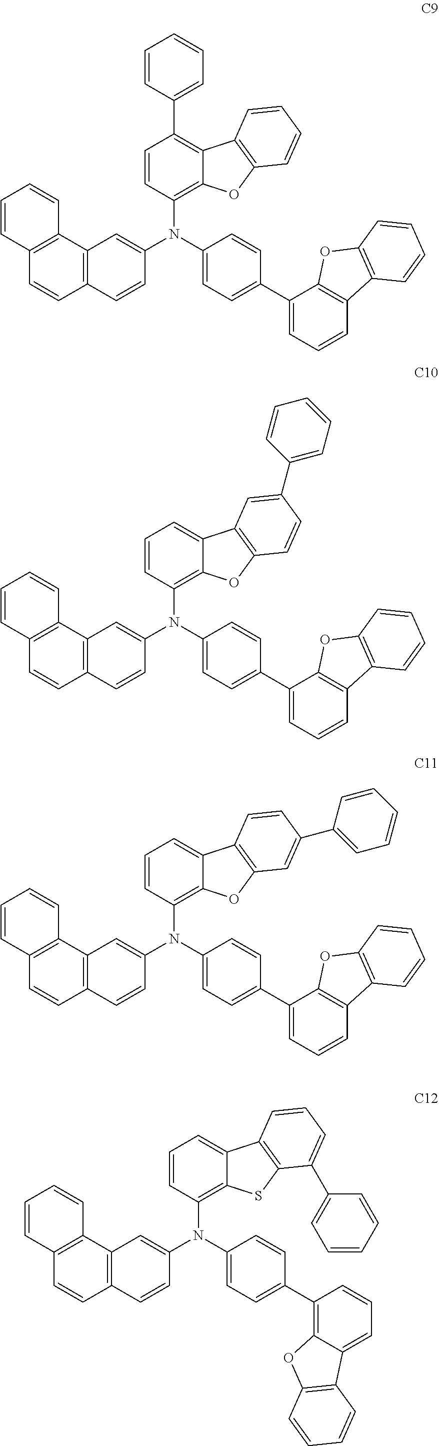 [SMILES: c1ccc(-c2ccc(N(c3ccc(-c4cccc5c4oc4ccccc45)cc3)c3ccc4ccc5ccccc5c4c3)c3oc4ccccc4c23)cc1 ; c1ccc(-c2ccc3oc4c(N(c5ccc(-c6cccc7c6oc6ccccc67)cc5)c5ccc6ccc7ccccc7c6c5)cccc4c3c2)cc1 ; c1ccc(-c2ccc3c(c2)oc2c(N(c4ccc(-c5cccc6c5oc5ccccc56)cc4)c4ccc5ccc6ccccc6c5c4)cccc23)cc1 ; c1ccc(-c2cccc3c2sc2c(N(c4ccc(-c5cccc6c5oc5ccccc56)cc4)c4ccc5ccc6ccccc6c5c4)cccc23)cc1] 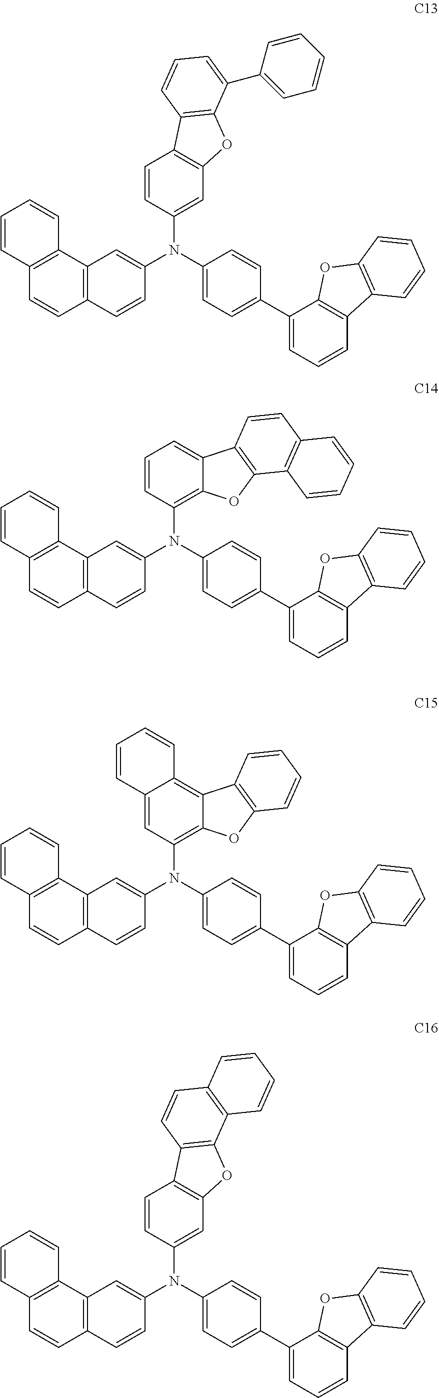 [SMILES: c1ccc(-c2cccc3c2oc2cc(N(c4ccc(-c5cccc6c5oc5ccccc56)cc4)c4ccc5ccc6ccccc6c5c4)ccc23)cc1 ; c1ccc2c(c1)ccc1ccc(N(c3ccc(-c4cccc5c4oc4ccccc45)cc3)c3cccc4c3oc3c5ccccc5ccc43)cc12 ; c1ccc2c(c1)ccc1ccc(N(c3ccc(-c4cccc5c4oc4ccccc45)cc3)c3cc4ccccc4c4c3oc3ccccc34)cc12 ; c1ccc2c(c1)ccc1ccc(N(c3ccc(-c4cccc5c4oc4ccccc45)cc3)c3ccc4c(c3)oc3c5ccccc5ccc43)cc12] 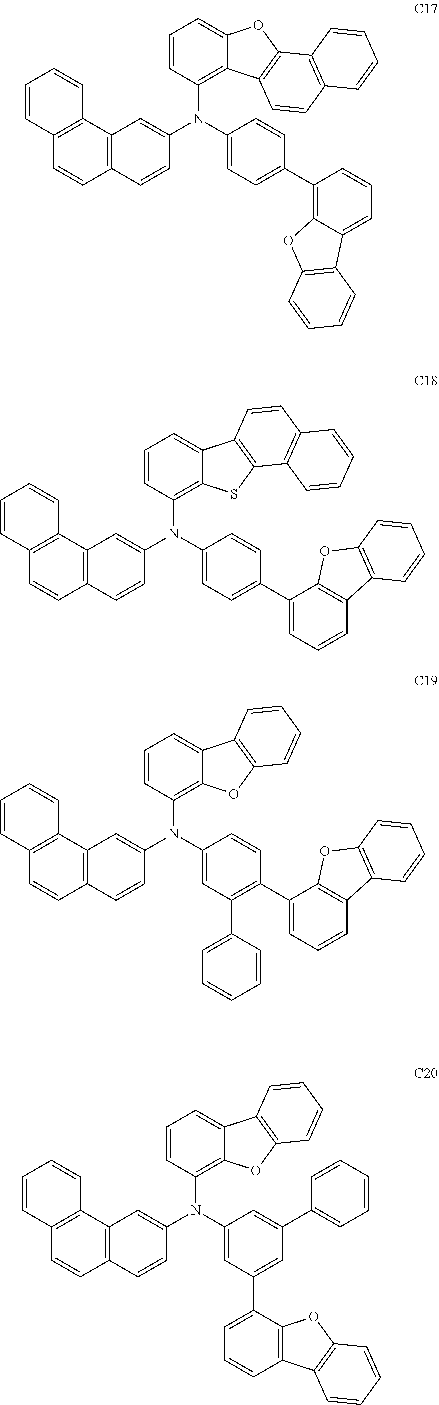 [SMILES: c1ccc2c(c1)ccc1ccc(N(c3ccc(-c4cccc5c4oc4ccccc45)cc3)c3cccc4oc5c6ccccc6ccc5c34)cc12 ; c1ccc2c(c1)ccc1ccc(N(c3ccc(-c4cccc5c4oc4ccccc45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2cccc3c2oc2ccccc23)cc1 ; c1ccc(-c2cc(-c3cccc4c3oc3ccccc34)cc(N(c3ccc4ccc5ccccc5c4c3)c3cccc4c3oc3ccccc34)c2)cc1] 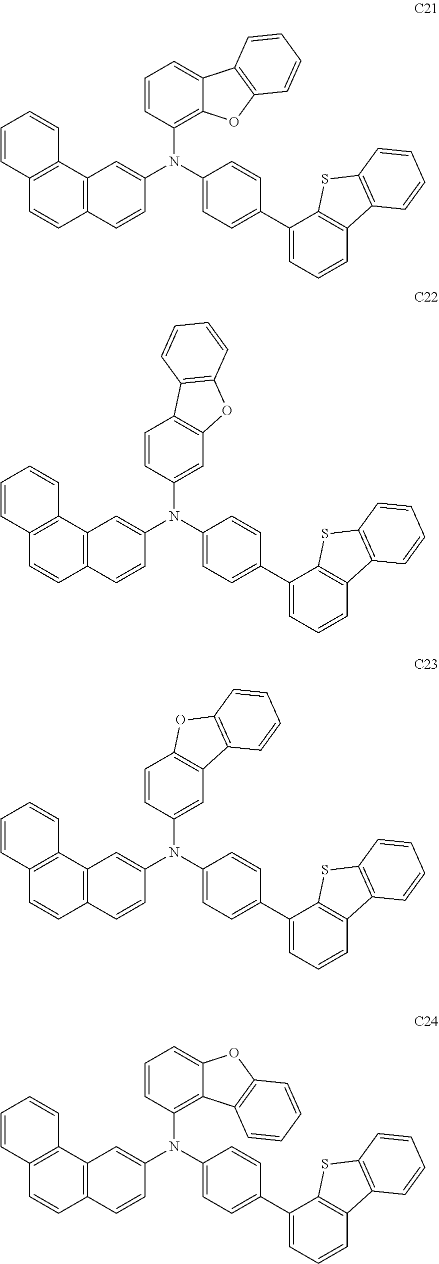 [SMILES: c1ccc2c(c1)ccc1ccc(N(c3ccc(-c4cccc5c4sc4ccccc45)cc3)c3cccc4c3oc3ccccc34)cc12 ; c1ccc2c(c1)ccc1ccc(N(c3ccc(-c4cccc5c4sc4ccccc45)cc3)c3ccc4c(c3)oc3ccccc34)cc12 ; c1ccc2c(c1)ccc1ccc(N(c3ccc(-c4cccc5c4sc4ccccc45)cc3)c3ccc4oc5ccccc5c4c3)cc12 ; c1ccc2c(c1)ccc1ccc(N(c3ccc(-c4cccc5c4sc4ccccc45)cc3)c3cccc4oc5ccccc5c34)cc12] 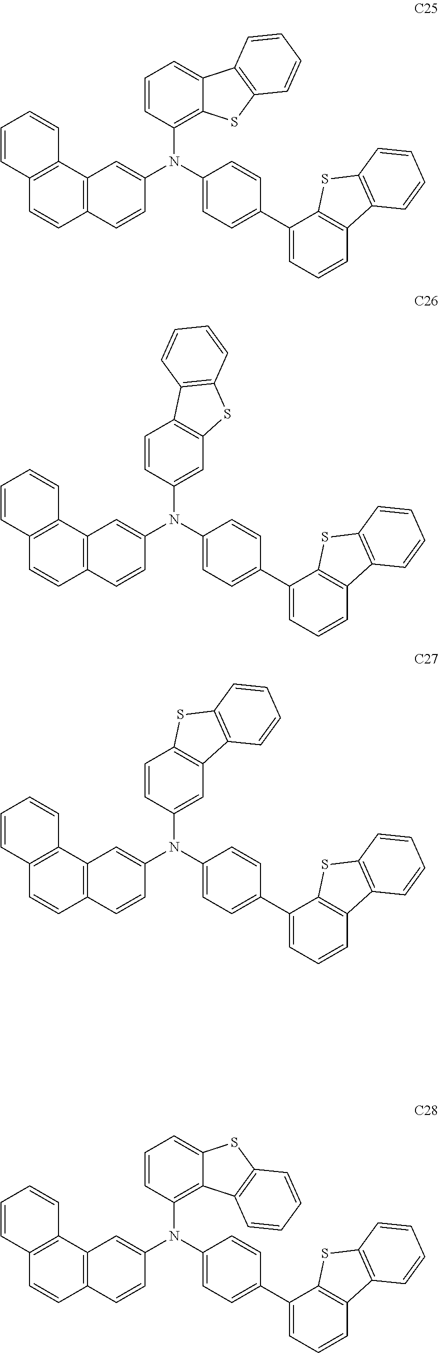 [SMILES: c1ccc2c(c1)ccc1ccc(N(c3ccc(-c4cccc5c4sc4ccccc45)cc3)c3cccc4c3sc3ccccc34)cc12 ; c1ccc2c(c1)ccc1ccc(N(c3ccc(-c4cccc5c4sc4ccccc45)cc3)c3ccc4c(c3)sc3ccccc34)cc12 ; c1ccc2c(c1)ccc1ccc(N(c3ccc(-c4cccc5c4sc4ccccc45)cc3)c3ccc4sc5ccccc5c4c3)cc12 ; c1ccc2c(c1)ccc1ccc(N(c3ccc(-c4cccc5c4sc4ccccc45)cc3)c3cccc4sc5ccccc5c34)cc12] 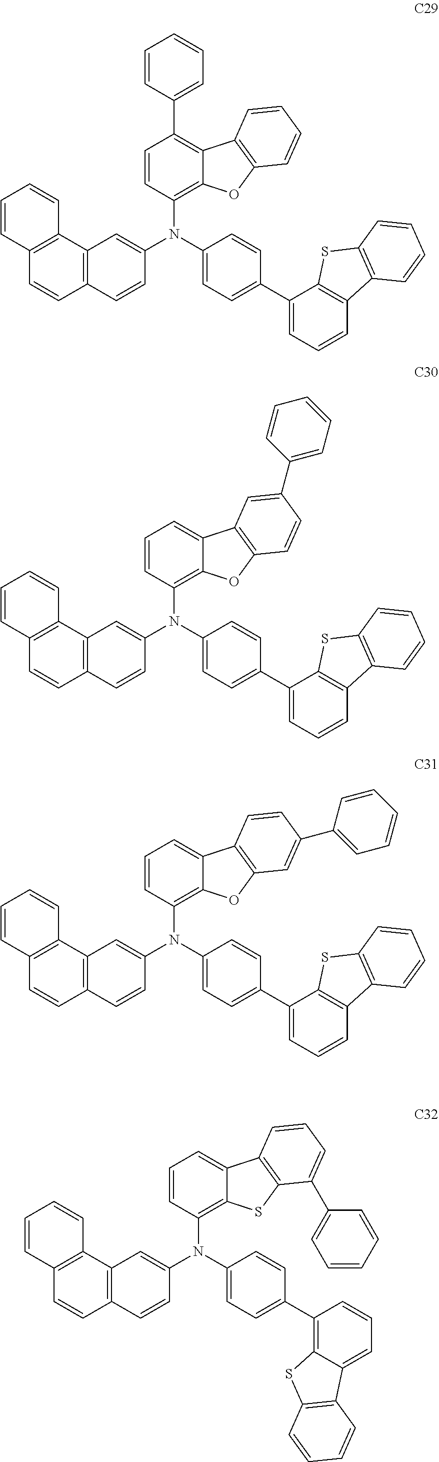 [SMILES: c1ccc(-c2ccc(N(c3ccc(-c4cccc5c4sc4ccccc45)cc3)c3ccc4ccc5ccccc5c4c3)c3oc4ccccc4c23)cc1 ; c1ccc(-c2ccc3oc4c(N(c5ccc(-c6cccc7c6sc6ccccc67)cc5)c5ccc6ccc7ccccc7c6c5)cccc4c3c2)cc1 ; c1ccc(-c2ccc3c(c2)oc2c(N(c4ccc(-c5cccc6c5sc5ccccc56)cc4)c4ccc5ccc6ccccc6c5c4)cccc23)cc1 ; c1ccc(-c2cccc3c2sc2c(N(c4ccc(-c5cccc6c5sc5ccccc56)cc4)c4ccc5ccc6ccccc6c5c4)cccc23)cc1] 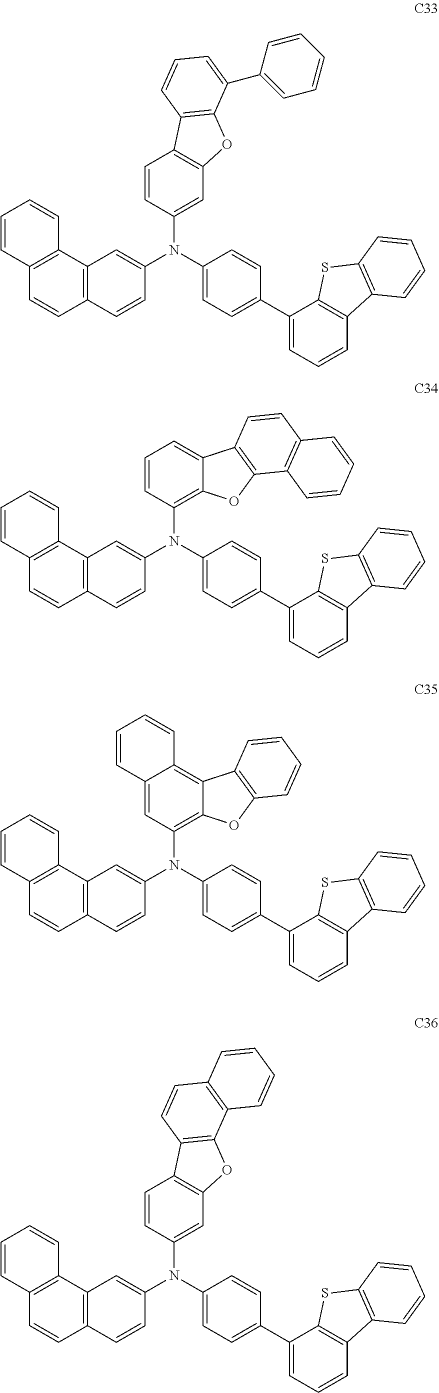 [SMILES: c1ccc(-c2cccc3c2oc2cc(N(c4ccc(-c5cccc6c5sc5ccccc56)cc4)c4ccc5ccc6ccccc6c5c4)ccc23)cc1 ; c1ccc2c(c1)ccc1ccc(N(c3ccc(-c4cccc5c4sc4ccccc45)cc3)c3cccc4c3oc3c5ccccc5ccc43)cc12 ; c1ccc2c(c1)ccc1ccc(N(c3ccc(-c4cccc5c4sc4ccccc45)cc3)c3cc4ccccc4c4c3oc3ccccc34)cc12 ; c1ccc2c(c1)ccc1ccc(N(c3ccc(-c4cccc5c4sc4ccccc45)cc3)c3ccc4c(c3)oc3c5ccccc5ccc43)cc12] 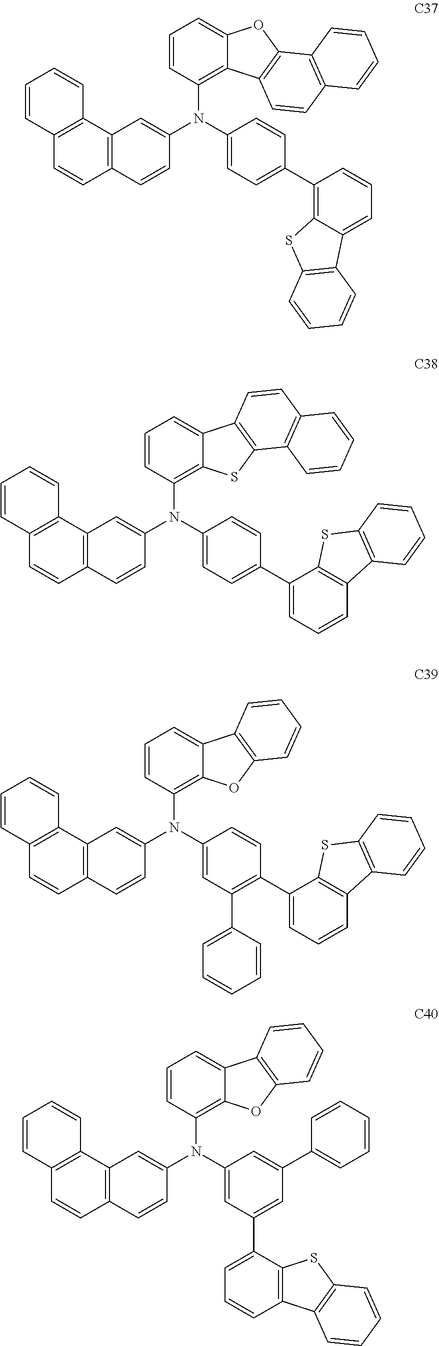 [SMILES: c1ccc2c(c1)ccc1ccc(N(c3ccc(-c4cccc5c4sc4ccccc45)cc3)c3cccc4oc5c6ccccc6ccc5c34)cc12 ; c1ccc2c(c1)ccc1ccc(N(c3ccc(-c4cccc5c4sc4ccccc45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2cccc3c2sc2ccccc23)cc1 ; c1ccc(-c2cc(-c3cccc4c3sc3ccccc34)cc(N(c3ccc4ccc5ccccc5c4c3)c3cccc4c3oc3ccccc34)c2)cc1] 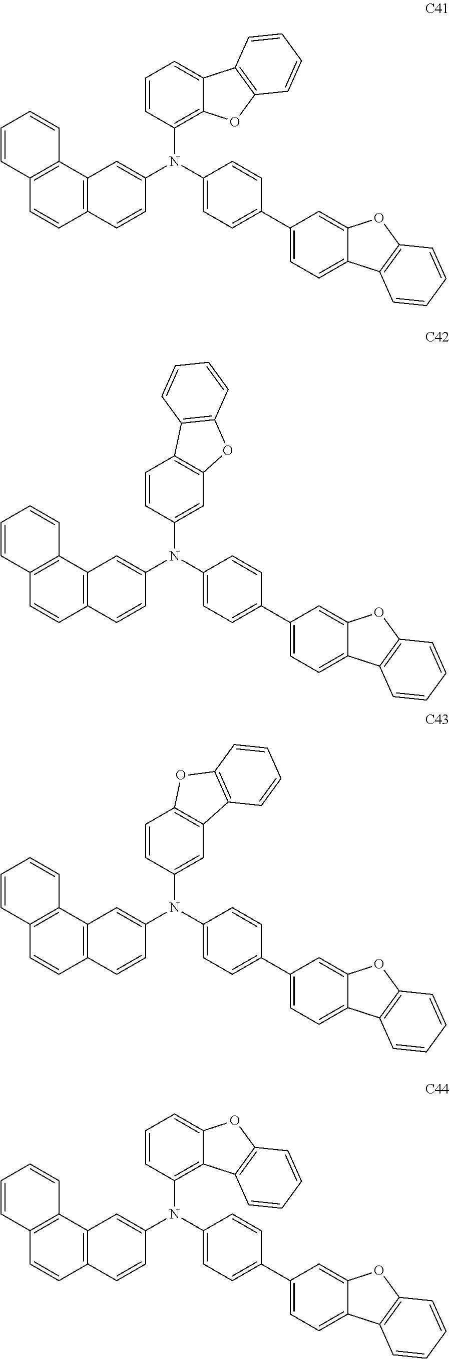 [SMILES: c1ccc2c(c1)ccc1ccc(N(c3ccc(-c4ccc5c(c4)oc4ccccc45)cc3)c3cccc4c3oc3ccccc34)cc12 ; c1ccc2c(c1)ccc1ccc(N(c3ccc(-c4ccc5c(c4)oc4ccccc45)cc3)c3ccc4c(c3)oc3ccccc34)cc12 ; c1ccc2c(c1)ccc1ccc(N(c3ccc(-c4ccc5c(c4)oc4ccccc45)cc3)c3ccc4oc5ccccc5c4c3)cc12 ; c1ccc2c(c1)ccc1ccc(N(c3ccc(-c4ccc5c(c4)oc4ccccc45)cc3)c3cccc4oc5ccccc5c34)cc12] 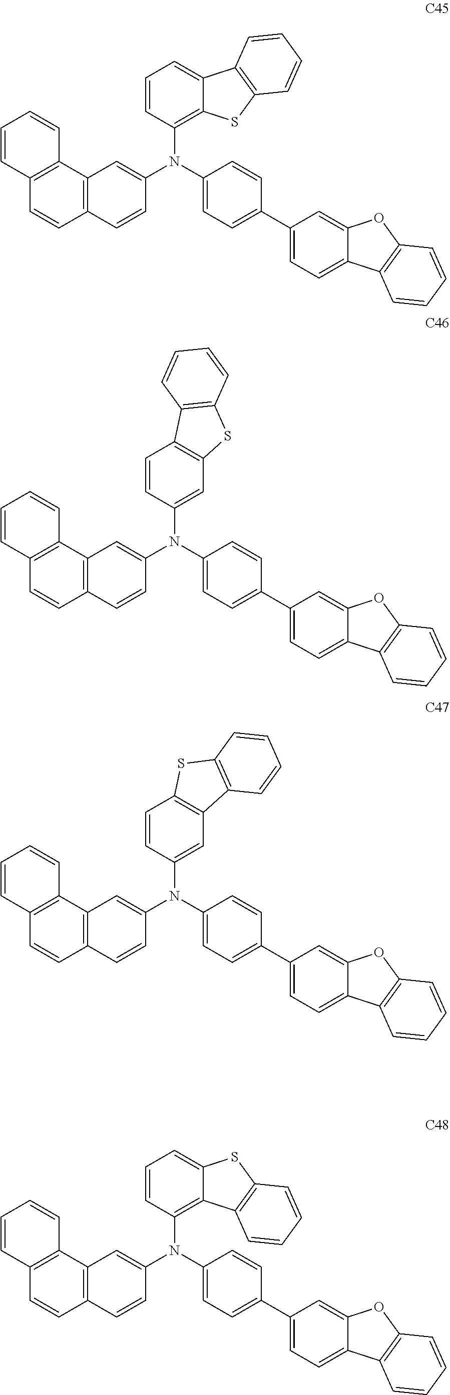 [SMILES: c1ccc2c(c1)ccc1ccc(N(c3ccc(-c4ccc5c(c4)oc4ccccc45)cc3)c3cccc4c3sc3ccccc34)cc12 ; c1ccc2c(c1)ccc1ccc(N(c3ccc(-c4ccc5c(c4)oc4ccccc45)cc3)c3ccc4c(c3)sc3ccccc34)cc12 ; c1ccc2c(c1)ccc1ccc(N(c3ccc(-c4ccc5c(c4)oc4ccccc45)cc3)c3ccc4sc5ccccc5c4c3)cc12 ; c1ccc2c(c1)ccc1ccc(N(c3ccc(-c4ccc5c(c4)oc4ccccc45)cc3)c3cccc4sc5ccccc5c34)cc12] 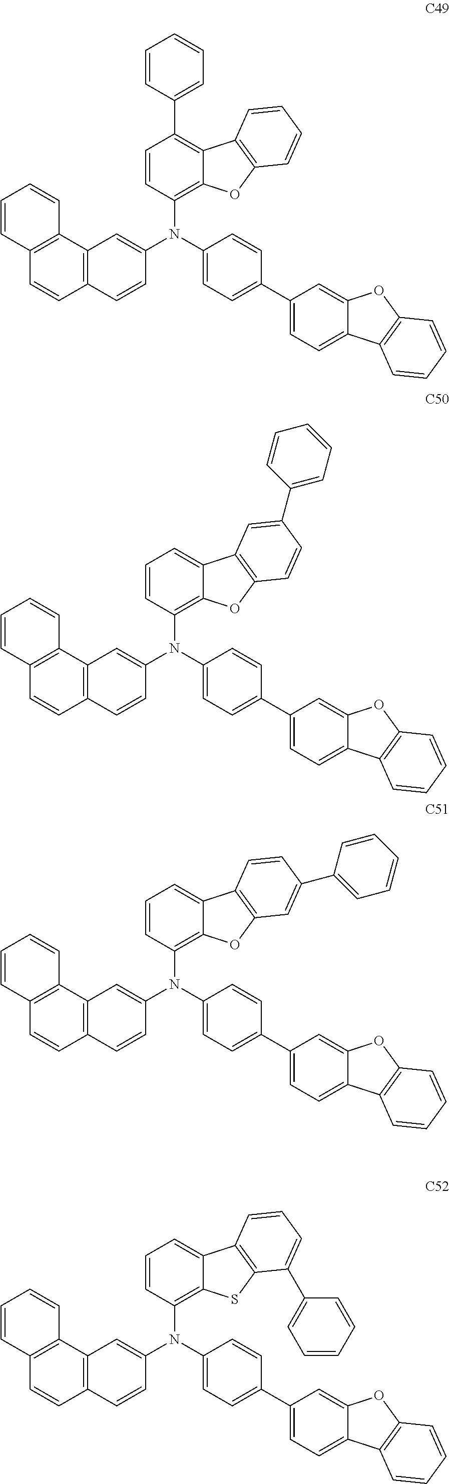 [SMILES: c1ccc(-c2ccc(N(c3ccc(-c4ccc5c(c4)oc4ccccc45)cc3)c3ccc4ccc5ccccc5c4c3)c3oc4ccccc4c23)cc1 ; c1ccc(-c2ccc3oc4c(N(c5ccc(-c6ccc7c(c6)oc6ccccc67)cc5)c5ccc6ccc7ccccc7c6c5)cccc4c3c2)cc1 ; c1ccc(-c2ccc3c(c2)oc2c(N(c4ccc(-c5ccc6c(c5)oc5ccccc56)cc4)c4ccc5ccc6ccccc6c5c4)cccc23)cc1 ; c1ccc(-c2cccc3c2sc2c(N(c4ccc(-c5ccc6c(c5)oc5ccccc56)cc4)c4ccc5ccc6ccccc6c5c4)cccc23)cc1] 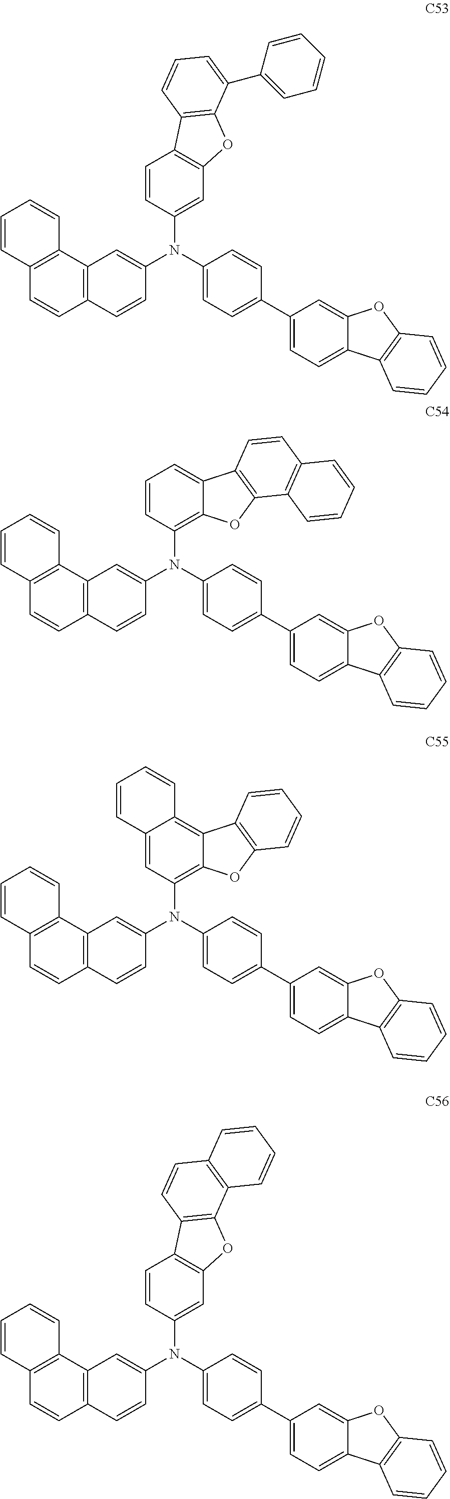 [SMILES: c1ccc(-c2cccc3c2oc2cc(N(c4ccc(-c5ccc6c(c5)oc5ccccc56)cc4)c4ccc5ccc6ccccc6c5c4)ccc23)cc1 ; c1ccc2c(c1)ccc1ccc(N(c3ccc(-c4ccc5c(c4)oc4ccccc45)cc3)c3cccc4c3oc3c5ccccc5ccc43)cc12 ; c1ccc2c(c1)ccc1ccc(N(c3ccc(-c4ccc5c(c4)oc4ccccc45)cc3)c3cc4ccccc4c4c3oc3ccccc34)cc12 ; c1ccc2c(c1)ccc1ccc(N(c3ccc(-c4ccc5c(c4)oc4ccccc45)cc3)c3ccc4c(c3)oc3c5ccccc5ccc43)cc12] 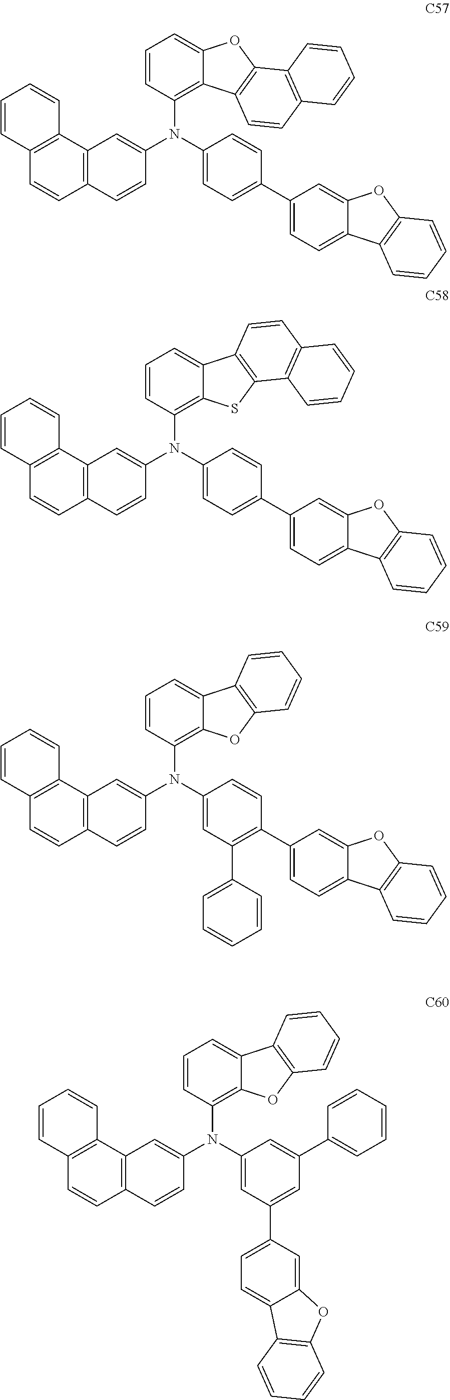 [SMILES: c1ccc2c(c1)ccc1ccc(N(c3ccc(-c4ccc5c(c4)oc4ccccc45)cc3)c3cccc4oc5c6ccccc6ccc5c34)cc12 ; c1ccc2c(c1)ccc1ccc(N(c3ccc(-c4ccc5c(c4)oc4ccccc45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2ccc3c(c2)oc2ccccc23)cc1 ; c1ccc(-c2cc(-c3ccc4c(c3)oc3ccccc34)cc(N(c3ccc4ccc5ccccc5c4c3)c3cccc4c3oc3ccccc34)c2)cc1] 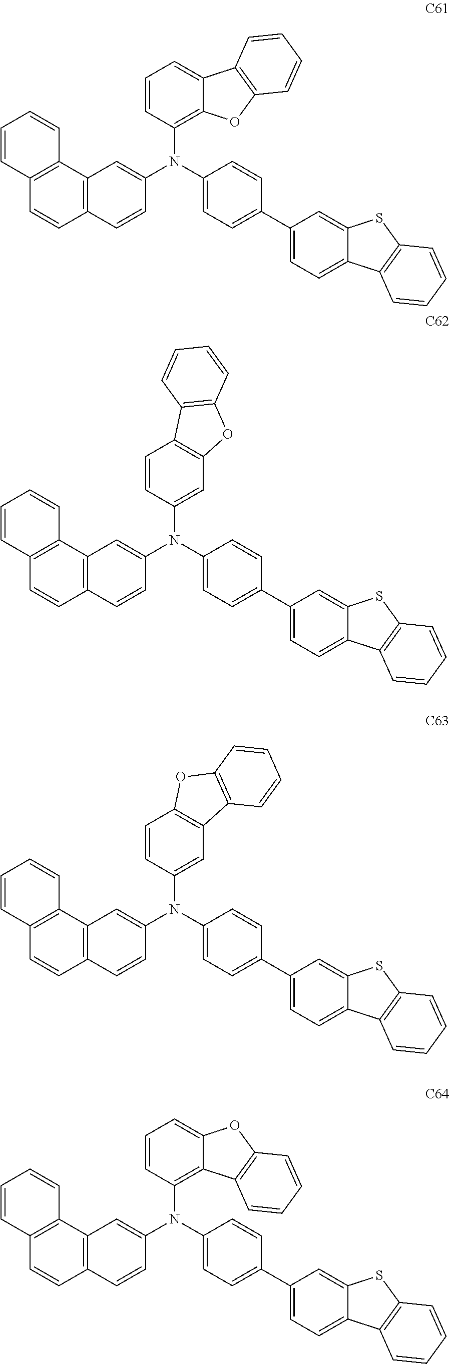 [SMILES: c1ccc2c(c1)ccc1ccc(N(c3ccc(-c4ccc5c(c4)sc4ccccc45)cc3)c3cccc4c3oc3ccccc34)cc12 ; c1ccc2c(c1)ccc1ccc(N(c3ccc(-c4ccc5c(c4)sc4ccccc45)cc3)c3ccc4c(c3)oc3ccccc34)cc12 ; c1ccc2c(c1)ccc1ccc(N(c3ccc(-c4ccc5c(c4)sc4ccccc45)cc3)c3ccc4oc5ccccc5c4c3)cc12 ; c1ccc2c(c1)ccc1ccc(N(c3ccc(-c4ccc5c(c4)sc4ccccc45)cc3)c3cccc4oc5ccccc5c34)cc12] 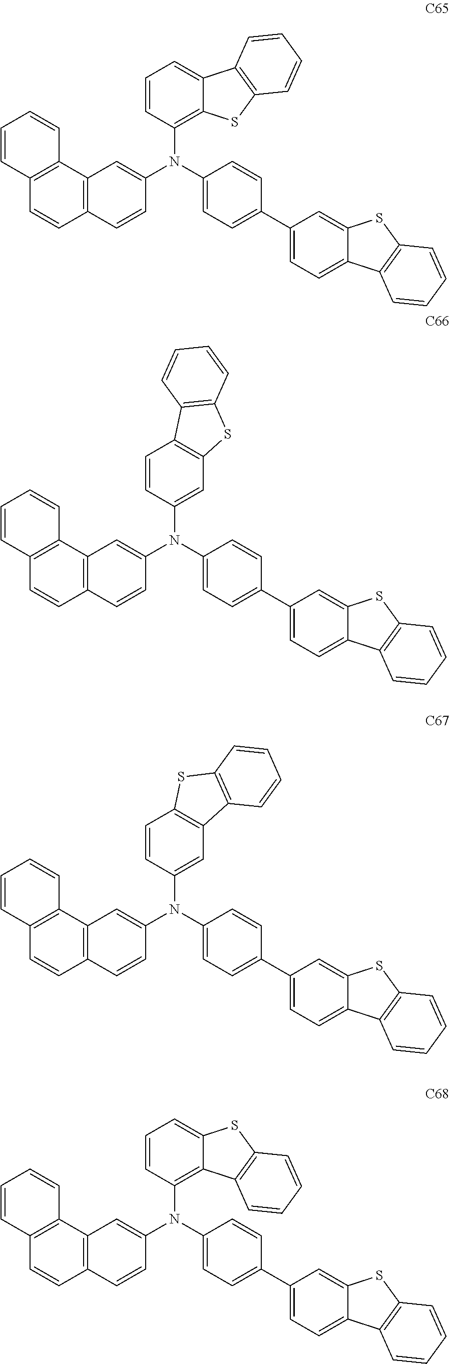 [SMILES: c1ccc2c(c1)ccc1ccc(N(c3ccc(-c4ccc5c(c4)sc4ccccc45)cc3)c3cccc4c3sc3ccccc34)cc12 ; c1ccc2c(c1)ccc1ccc(N(c3ccc(-c4ccc5c(c4)sc4ccccc45)cc3)c3ccc4c(c3)sc3ccccc34)cc12 ; c1ccc2c(c1)ccc1ccc(N(c3ccc(-c4ccc5c(c4)sc4ccccc45)cc3)c3ccc4sc5ccccc5c4c3)cc12 ; c1ccc2c(c1)ccc1ccc(N(c3ccc(-c4ccc5c(c4)sc4ccccc45)cc3)c3cccc4sc5ccccc5c34)cc12] 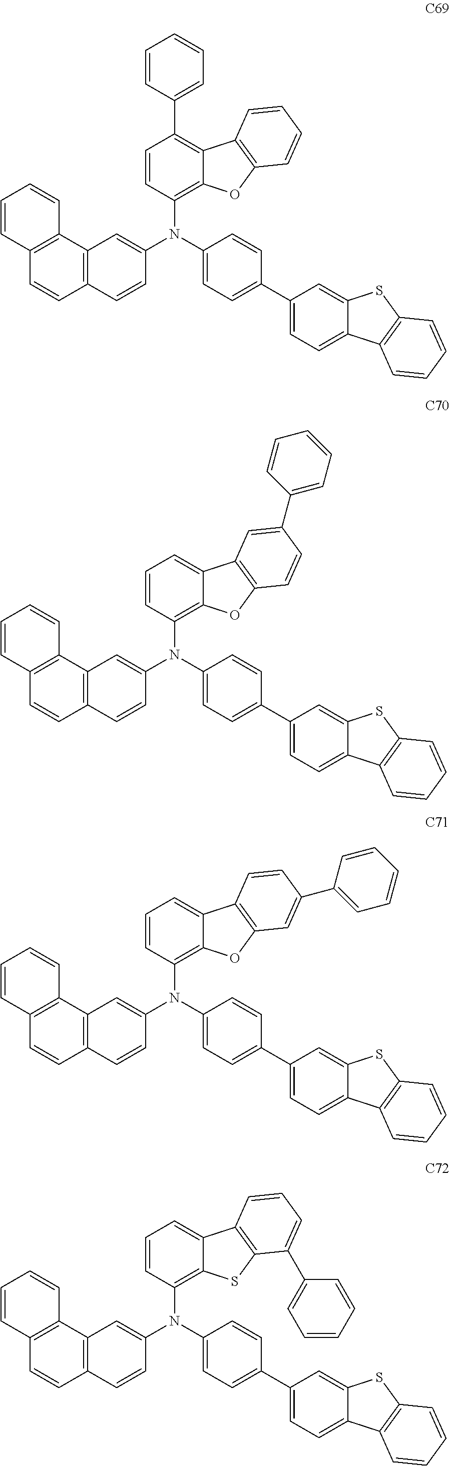 [SMILES: c1ccc(-c2ccc(N(c3ccc(-c4ccc5c(c4)sc4ccccc45)cc3)c3ccc4ccc5ccccc5c4c3)c3oc4ccccc4c23)cc1 ; c1ccc(-c2ccc3oc4c(N(c5ccc(-c6ccc7c(c6)sc6ccccc67)cc5)c5ccc6ccc7ccccc7c6c5)cccc4c3c2)cc1 ; c1ccc(-c2ccc3c(c2)oc2c(N(c4ccc(-c5ccc6c(c5)sc5ccccc56)cc4)c4ccc5ccc6ccccc6c5c4)cccc23)cc1 ; c1ccc(-c2cccc3c2sc2c(N(c4ccc(-c5ccc6c(c5)sc5ccccc56)cc4)c4ccc5ccc6ccccc6c5c4)cccc23)cc1] 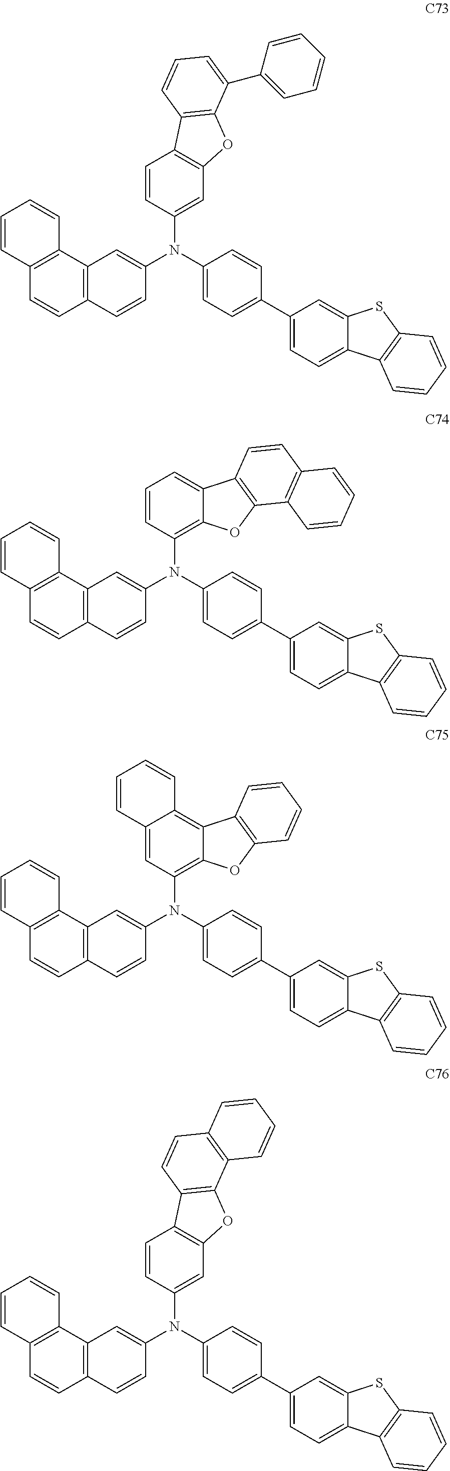 [SMILES: c1ccc(-c2cccc3c2oc2cc(N(c4ccc(-c5ccc6c(c5)sc5ccccc56)cc4)c4ccc5ccc6ccccc6c5c4)ccc23)cc1 ; c1ccc2c(c1)ccc1ccc(N(c3ccc(-c4ccc5c(c4)sc4ccccc45)cc3)c3cccc4c3oc3c5ccccc5ccc43)cc12 ; c1ccc2c(c1)ccc1ccc(N(c3ccc(-c4ccc5c(c4)sc4ccccc45)cc3)c3cc4ccccc4c4c3oc3ccccc34)cc12 ; c1ccc2c(c1)ccc1ccc(N(c3ccc(-c4ccc5c(c4)sc4ccccc45)cc3)c3ccc4c(c3)oc3c5ccccc5ccc43)cc12] 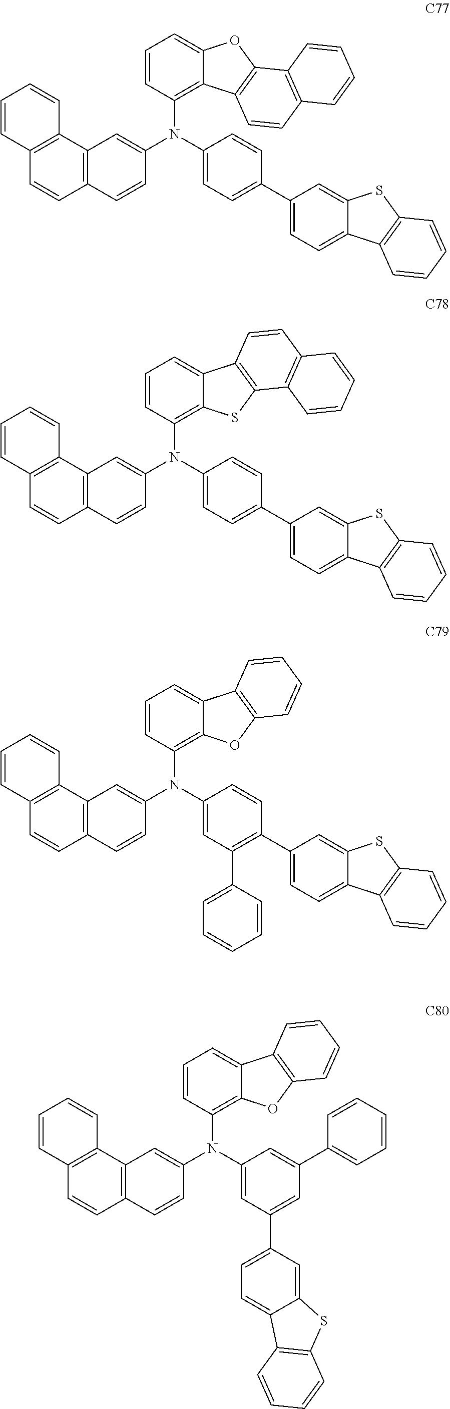 [SMILES: c1ccc2c(c1)ccc1ccc(N(c3ccc(-c4ccc5c(c4)sc4ccccc45)cc3)c3cccc4oc5c6ccccc6ccc5c34)cc12 ; c1ccc2c(c1)ccc1ccc(N(c3ccc(-c4ccc5c(c4)sc4ccccc45)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2ccc3c(c2)sc2ccccc23)cc1 ; c1ccc(-c2cc(-c3ccc4c(c3)sc3ccccc34)cc(N(c3ccc4ccc5ccccc5c4c3)c3cccc4c3oc3ccccc34)c2)cc1] 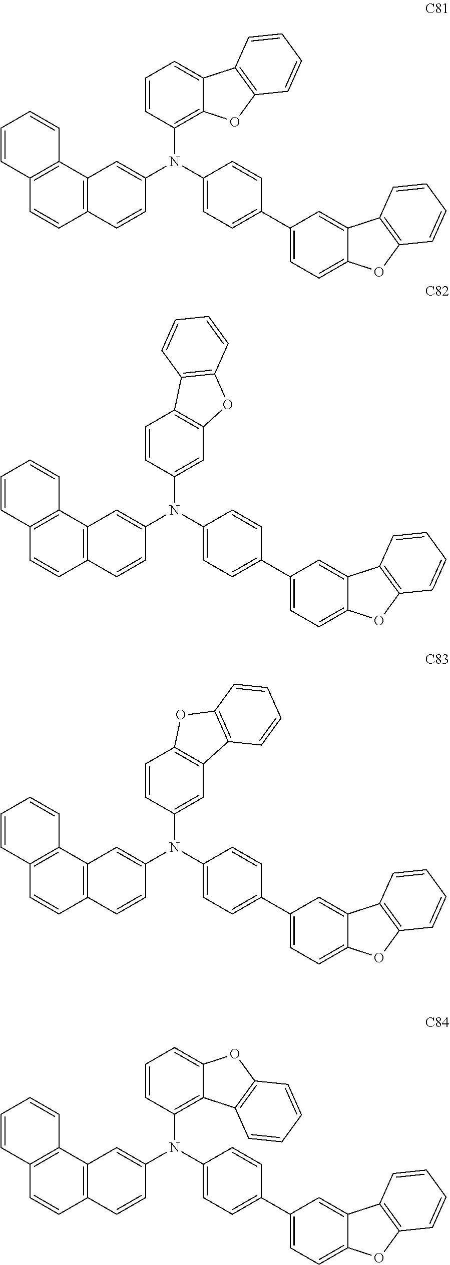 [SMILES: c1ccc2c(c1)ccc1ccc(N(c3ccc(-c4ccc5oc6ccccc6c5c4)cc3)c3cccc4c3oc3ccccc34)cc12 ; c1ccc2c(c1)ccc1ccc(N(c3ccc(-c4ccc5oc6ccccc6c5c4)cc3)c3ccc4c(c3)oc3ccccc34)cc12 ; c1ccc2c(c1)ccc1ccc(N(c3ccc(-c4ccc5oc6ccccc6c5c4)cc3)c3ccc4oc5ccccc5c4c3)cc12 ; c1ccc2c(c1)ccc1ccc(N(c3ccc(-c4ccc5oc6ccccc6c5c4)cc3)c3cccc4oc5ccccc5c34)cc12] 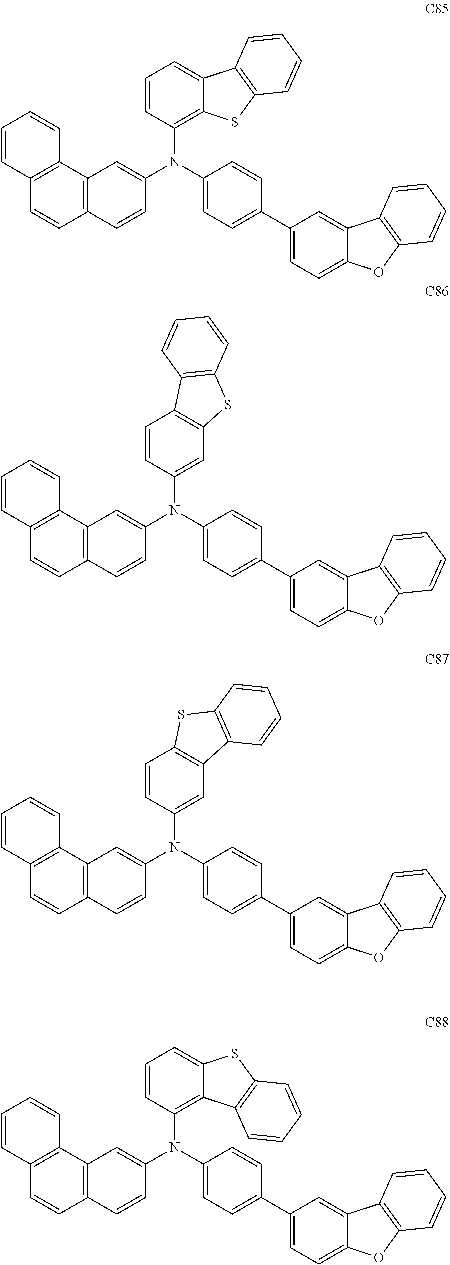 [SMILES: c1ccc2c(c1)ccc1ccc(N(c3ccc(-c4ccc5oc6ccccc6c5c4)cc3)c3cccc4c3sc3ccccc34)cc12 ; c1ccc2c(c1)ccc1ccc(N(c3ccc(-c4ccc5oc6ccccc6c5c4)cc3)c3ccc4c(c3)sc3ccccc34)cc12 ; c1ccc2c(c1)ccc1ccc(N(c3ccc(-c4ccc5oc6ccccc6c5c4)cc3)c3ccc4sc5ccccc5c4c3)cc12 ; c1ccc2c(c1)ccc1ccc(N(c3ccc(-c4ccc5oc6ccccc6c5c4)cc3)c3cccc4sc5ccccc5c34)cc12] 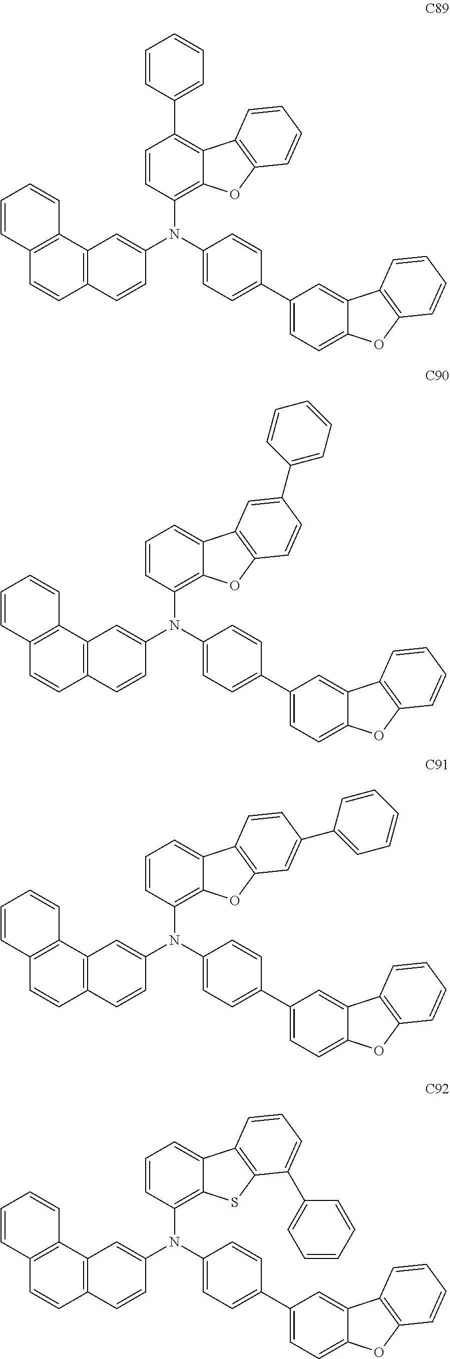 [SMILES: c1ccc(-c2ccc(N(c3ccc(-c4ccc5oc6ccccc6c5c4)cc3)c3ccc4ccc5ccccc5c4c3)c3oc4ccccc4c23)cc1 ; c1ccc(-c2ccc3oc4c(N(c5ccc(-c6ccc7oc8ccccc8c7c6)cc5)c5ccc6ccc7ccccc7c6c5)cccc4c3c2)cc1 ; c1ccc(-c2ccc3c(c2)oc2c(N(c4ccc(-c5ccc6oc7ccccc7c6c5)cc4)c4ccc5ccc6ccccc6c5c4)cccc23)cc1 ; c1ccc(-c2cccc3c2sc2c(N(c4ccc(-c5ccc6oc7ccccc7c6c5)cc4)c4ccc5ccc6ccccc6c5c4)cccc23)cc1] 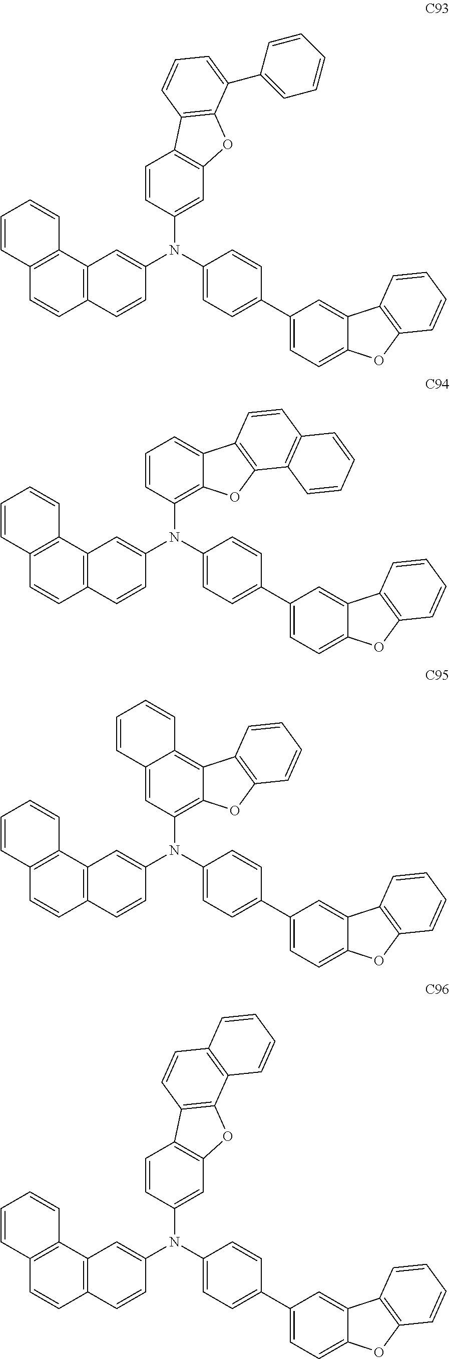 [SMILES: c1ccc(-c2cccc3c2oc2cc(N(c4ccc(-c5ccc6oc7ccccc7c6c5)cc4)c4ccc5ccc6ccccc6c5c4)ccc23)cc1 ; c1ccc2c(c1)ccc1ccc(N(c3ccc(-c4ccc5oc6ccccc6c5c4)cc3)c3cccc4c3oc3c5ccccc5ccc43)cc12 ; c1ccc2c(c1)ccc1ccc(N(c3ccc(-c4ccc5oc6ccccc6c5c4)cc3)c3cc4ccccc4c4c3oc3ccccc34)cc12 ; c1ccc2c(c1)ccc1ccc(N(c3ccc(-c4ccc5oc6ccccc6c5c4)cc3)c3ccc4c(c3)oc3c5ccccc5ccc43)cc12] 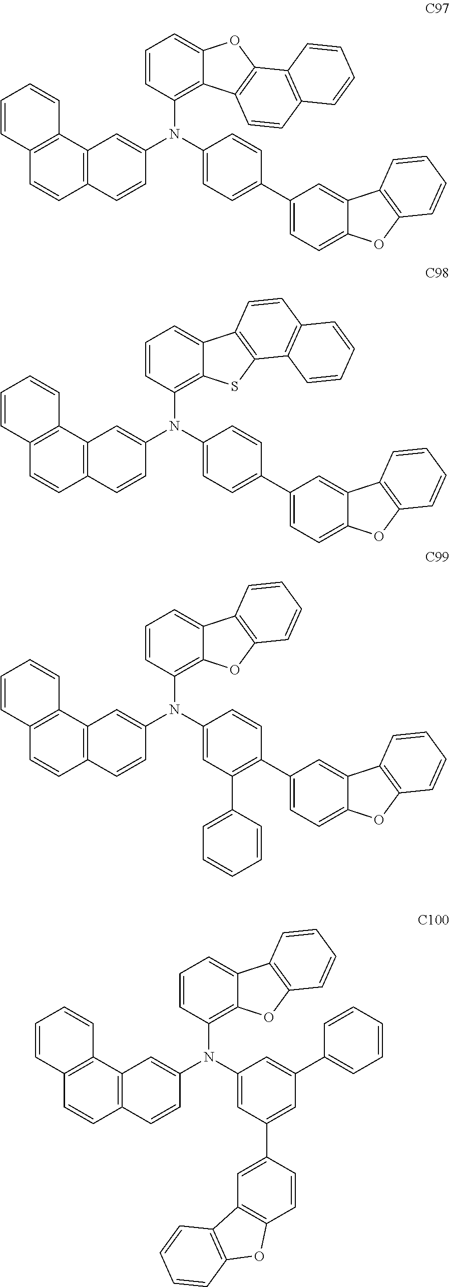 [SMILES: c1ccc2c(c1)ccc1ccc(N(c3ccc(-c4ccc5oc6ccccc6c5c4)cc3)c3cccc4oc5c6ccccc6ccc5c34)cc12 ; c1ccc2c(c1)ccc1ccc(N(c3ccc(-c4ccc5oc6ccccc6c5c4)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2ccc3oc4ccccc4c3c2)cc1 ; c1ccc(-c2cc(-c3ccc4oc5ccccc5c4c3)cc(N(c3ccc4ccc5ccccc5c4c3)c3cccc4c3oc3ccccc34)c2)cc1] 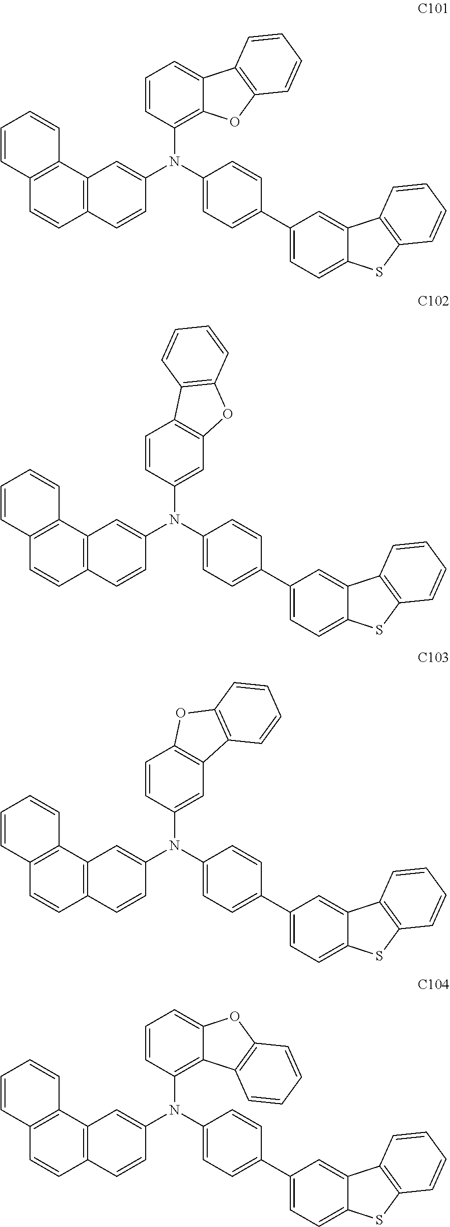 [SMILES: c1ccc2c(c1)ccc1ccc(N(c3ccc(-c4ccc5sc6ccccc6c5c4)cc3)c3cccc4c3oc3ccccc34)cc12 ; c1ccc2c(c1)ccc1ccc(N(c3ccc(-c4ccc5sc6ccccc6c5c4)cc3)c3ccc4c(c3)oc3ccccc34)cc12 ; c1ccc2c(c1)ccc1ccc(N(c3ccc(-c4ccc5sc6ccccc6c5c4)cc3)c3ccc4oc5ccccc5c4c3)cc12 ; c1ccc2c(c1)ccc1ccc(N(c3ccc(-c4ccc5sc6ccccc6c5c4)cc3)c3cccc4oc5ccccc5c34)cc12] 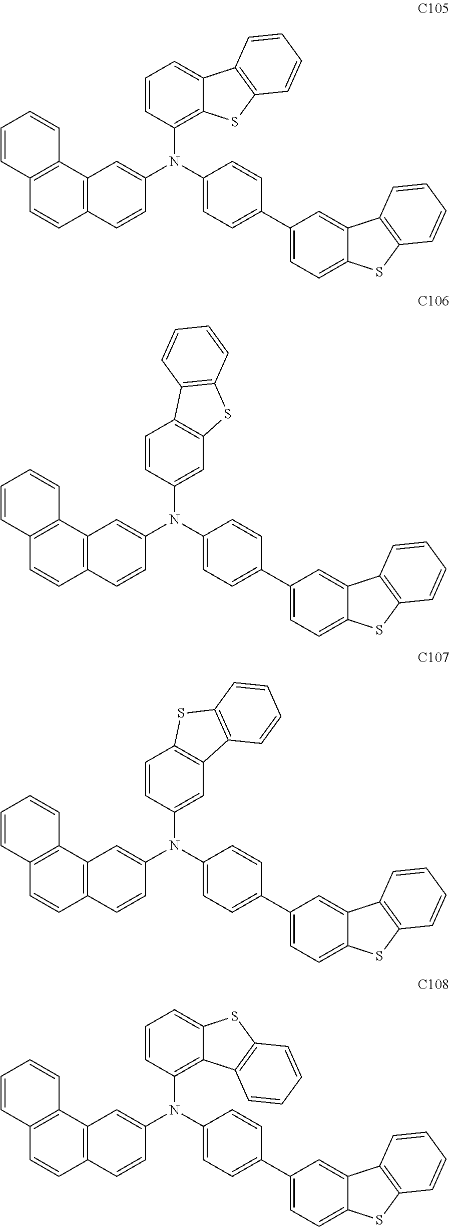 [SMILES: c1ccc2c(c1)ccc1ccc(N(c3ccc(-c4ccc5sc6ccccc6c5c4)cc3)c3cccc4c3sc3ccccc34)cc12 ; c1ccc2c(c1)ccc1ccc(N(c3ccc(-c4ccc5sc6ccccc6c5c4)cc3)c3ccc4c(c3)sc3ccccc34)cc12 ; c1ccc2c(c1)ccc1ccc(N(c3ccc(-c4ccc5sc6ccccc6c5c4)cc3)c3ccc4sc5ccccc5c4c3)cc12 ; c1ccc2c(c1)ccc1ccc(N(c3ccc(-c4ccc5sc6ccccc6c5c4)cc3)c3cccc4sc5ccccc5c34)cc12] 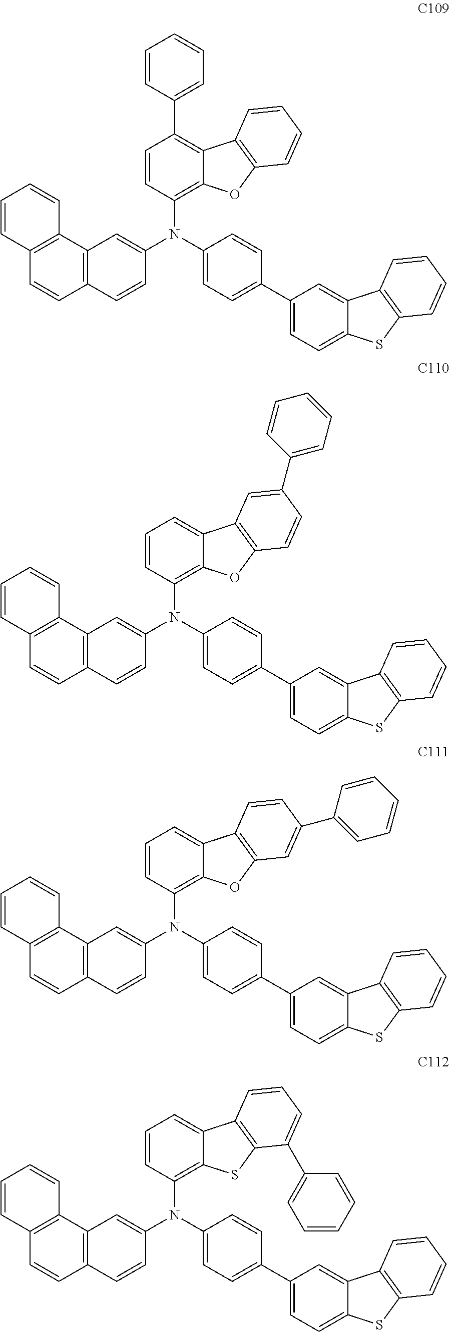 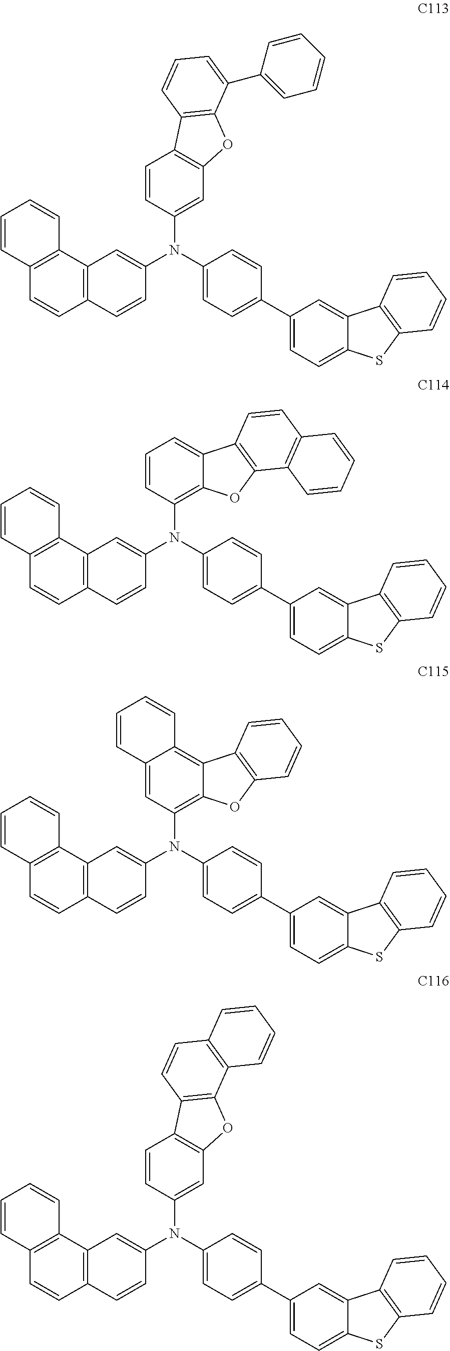 [SMILES: c1ccc(-c2cccc3c2oc2cc(N(c4ccc(-c5ccc6sc7ccccc7c6c5)cc4)c4ccc5ccc6ccccc6c5c4)ccc23)cc1 ; c1ccc2c(c1)ccc1ccc(N(c3ccc(-c4ccc5sc6ccccc6c5c4)cc3)c3cccc4c3oc3c5ccccc5ccc43)cc12 ; c1ccc2c(c1)ccc1ccc(N(c3ccc(-c4ccc5sc6ccccc6c5c4)cc3)c3cc4ccccc4c4c3oc3ccccc34)cc12 ; c1ccc2c(c1)ccc1ccc(N(c3ccc(-c4ccc5sc6ccccc6c5c4)cc3)c3ccc4c(c3)oc3c5ccccc5ccc43)cc12] 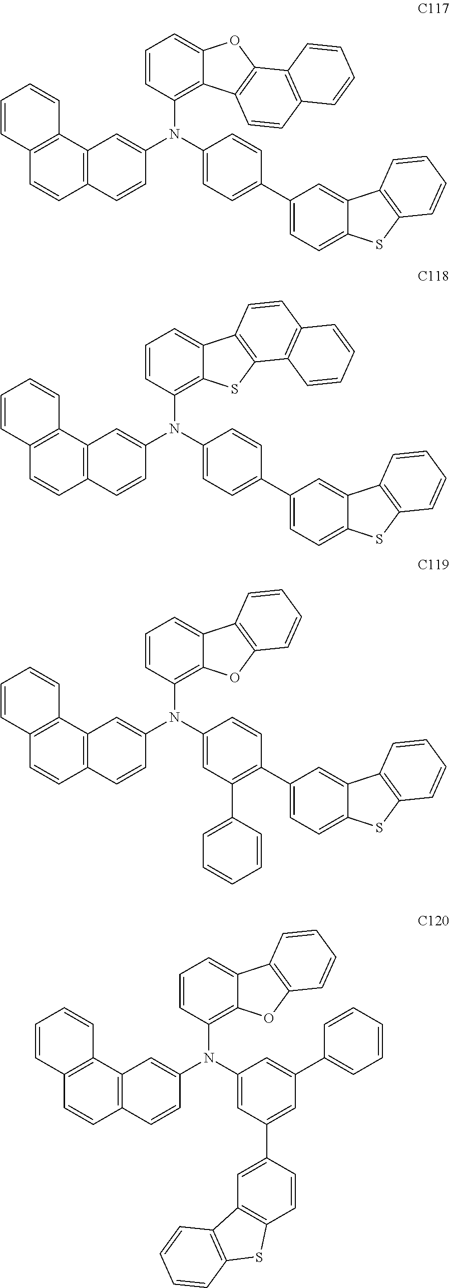 [SMILES: c1ccc2c(c1)ccc1ccc(N(c3ccc(-c4ccc5sc6ccccc6c5c4)cc3)c3cccc4oc5c6ccccc6ccc5c34)cc12 ; c1ccc2c(c1)ccc1ccc(N(c3ccc(-c4ccc5sc6ccccc6c5c4)cc3)c3cccc4c3sc3c5ccccc5ccc43)cc12 ; c1ccc(-c2cc(N(c3ccc4ccc5ccccc5c4c3)c3cccc4c3oc3ccccc34)ccc2-c2ccc3sc4ccccc4c3c2)cc1 ; c1ccc(-c2cc(-c3ccc4sc5ccccc5c4c3)cc(N(c3ccc4ccc5ccccc5c4c3)c3cccc4c3oc3ccccc34)c2)cc1] 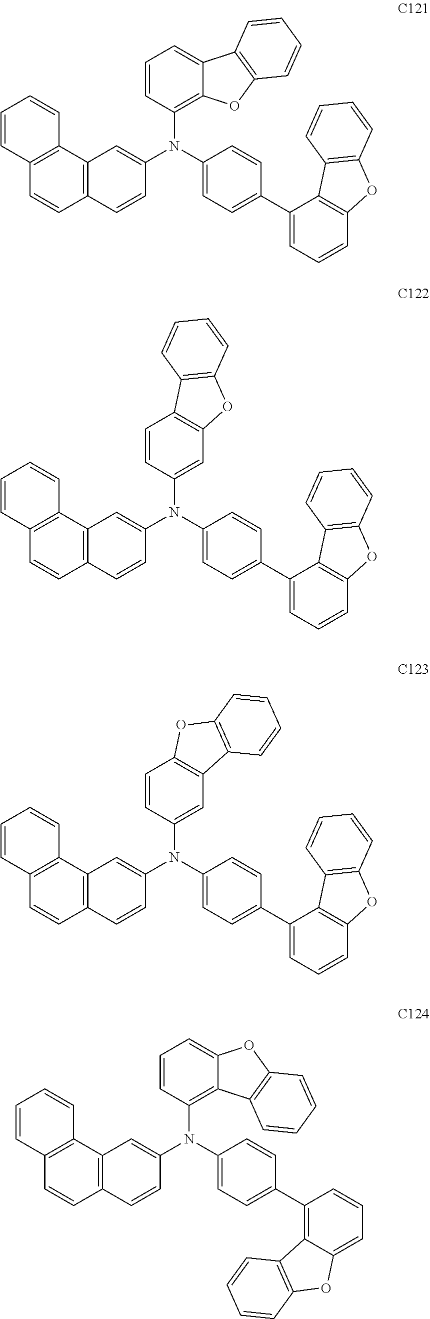 [SMILES: c1ccc2c(c1)ccc1ccc(N(c3ccc(-c4cccc5oc6ccccc6c45)cc3)c3cccc4c3oc3ccccc34)cc12 ; c1ccc2c(c1)ccc1ccc(N(c3ccc(-c4cccc5oc6ccccc6c45)cc3)c3ccc4c(c3)oc3ccccc34)cc12 ; c1ccc2c(c1)ccc1ccc(N(c3ccc(-c4cccc5oc6ccccc6c45)cc3)c3ccc4oc5ccccc5c4c3)cc12 ; c1ccc2c(c1)ccc1ccc(N(c3ccc(-c4cccc5oc6ccccc6c45)cc3)c3cccc4oc5ccccc5c34)cc12] 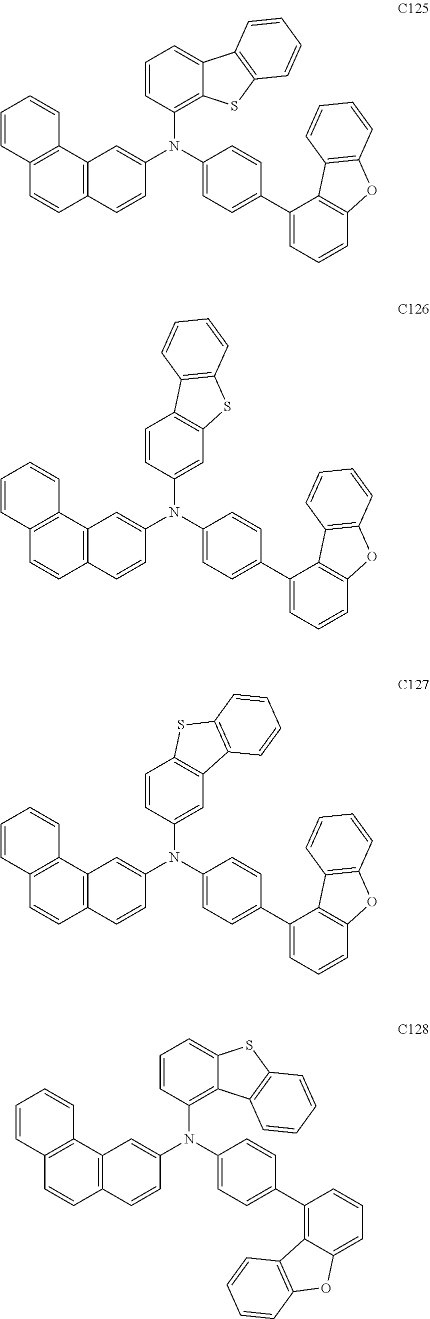 [SMILES: c1ccc2c(c1)ccc1ccc(N(c3ccc(-c4cccc5oc6ccccc6c45)cc3)c3cccc4c3sc3ccccc34)cc12 ; c1ccc2c(c1)ccc1ccc(N(c3ccc(-c4cccc5oc6ccccc6c45)cc3)c3ccc4c(c3)sc3ccccc34)cc12 ; c1ccc2c(c1)ccc1ccc(N(c3ccc(-c4cccc5oc6ccccc6c45)cc3)c3ccc4sc5ccccc5c4c3)cc12 ; c1ccc2c(c1)ccc1ccc(N(c3ccc(-c4cccc5oc6ccccc6c45)cc3)c3cccc4sc5ccccc5c34)cc12] 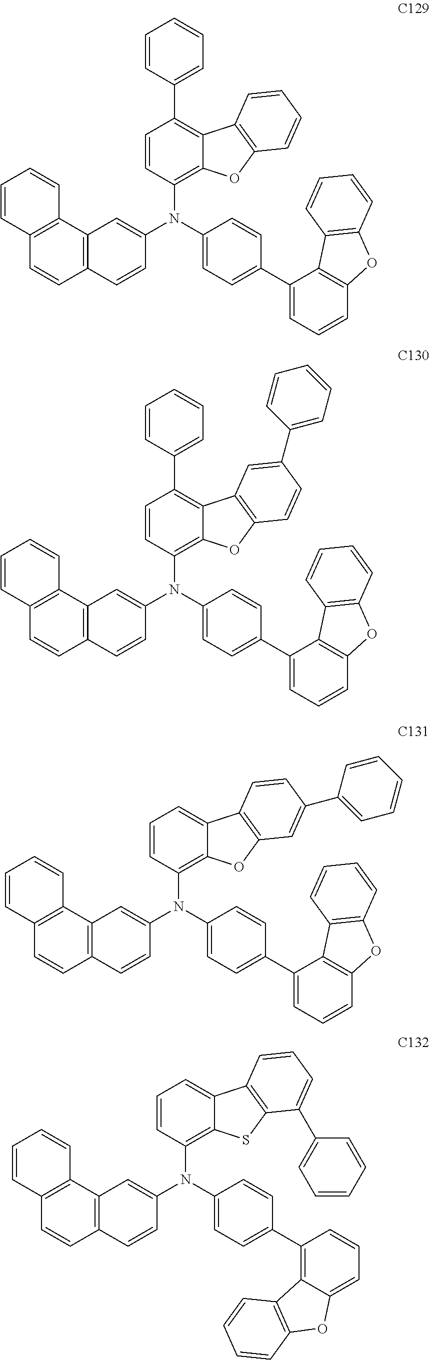 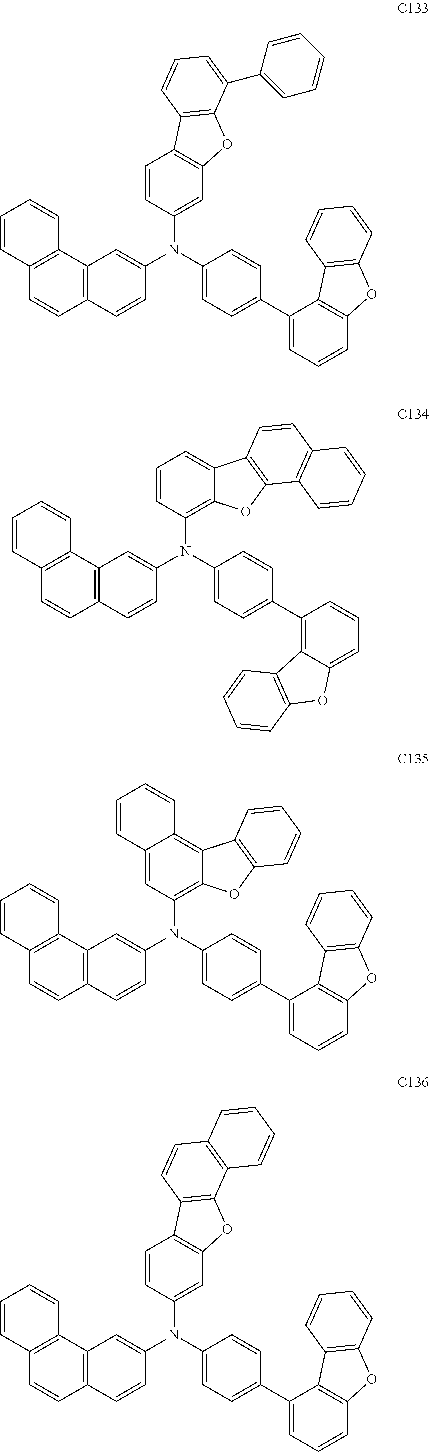 [SMILES: c1ccc(-c2cccc3c2oc2cc(N(c4ccc(-c5cccc6oc7ccccc7c56)cc4)c4ccc5ccc6ccccc6c5c4)ccc23)cc1 ; c1ccc2c(c1)ccc1ccc(N(c3ccc(-c4cccc5oc6ccccc6c45)cc3)c3cccc4c3oc3c5ccccc5ccc43)cc12 ; c1ccc2c(c1)ccc1ccc(N(c3ccc(-c4cccc5oc6ccccc6c45)cc3)c3cc4ccccc4c4c3oc3ccccc34)cc12 ; c1ccc2c(c1)ccc1ccc(N(c3ccc(-c4cccc5oc6ccccc6c45)cc3)c3ccc4c(c3)oc3c5ccccc5ccc43)cc12] 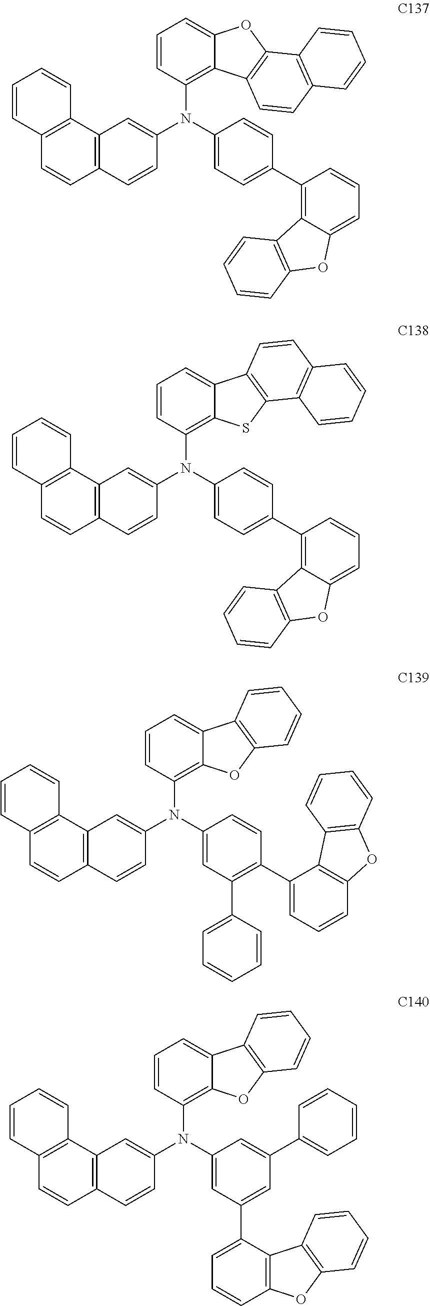 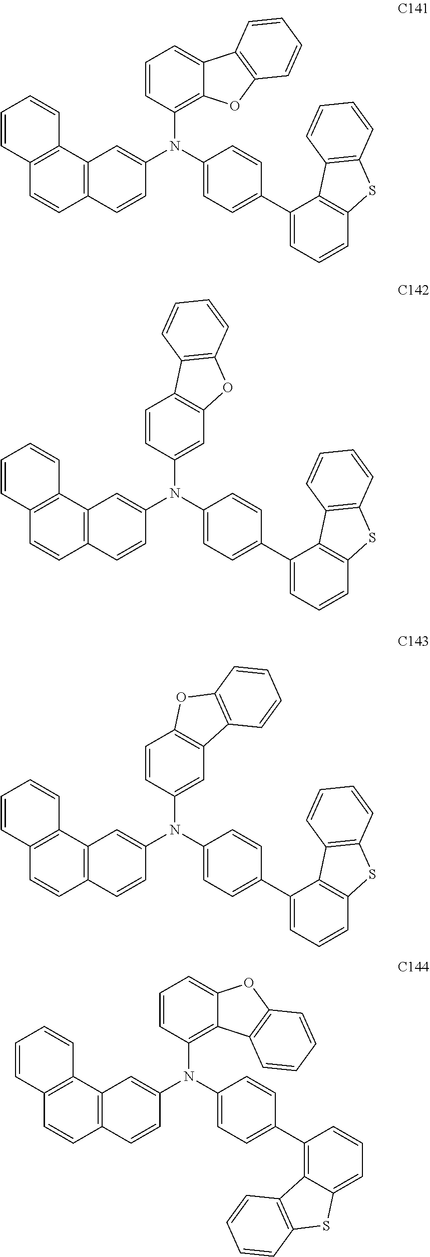 [SMILES: c1ccc2c(c1)ccc1ccc(N(c3ccc(-c4cccc5sc6ccccc6c45)cc3)c3cccc4c3oc3ccccc34)cc12 ; c1ccc2c(c1)ccc1ccc(N(c3ccc(-c4cccc5sc6ccccc6c45)cc3)c3ccc4c(c3)oc3ccccc34)cc12 ; c1ccc2c(c1)ccc1ccc(N(c3ccc(-c4cccc5sc6ccccc6c45)cc3)c3ccc4oc5ccccc5c4c3)cc12 ; c1ccc2c(c1)ccc1ccc(N(c3ccc(-c4cccc5sc6ccccc6c45)cc3)c3cccc4oc5ccccc5c34)cc12] 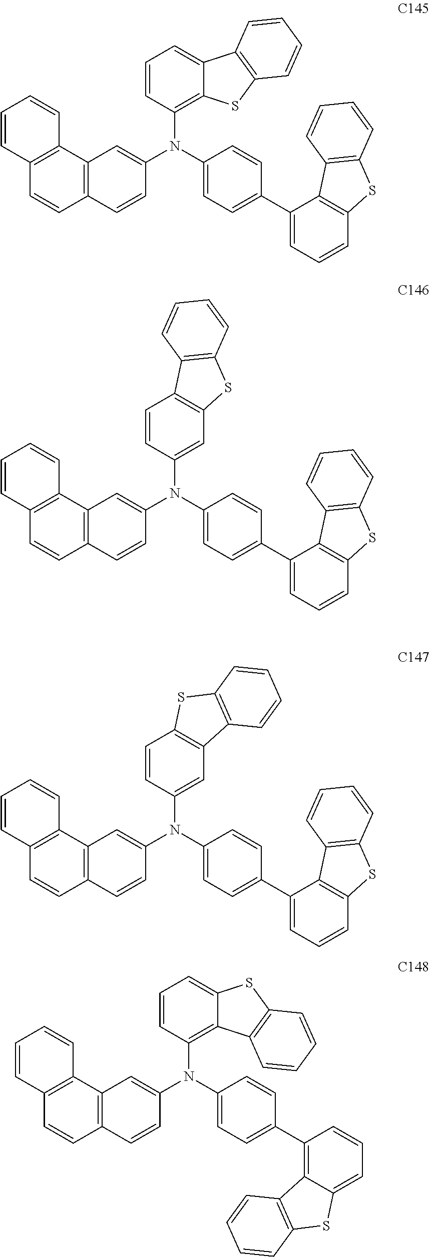 [SMILES: c1ccc2c(c1)ccc1ccc(N(c3ccc(-c4cccc5sc6ccccc6c45)cc3)c3cccc4c3sc3ccccc34)cc12 ; c1ccc2c(c1)ccc1ccc(N(c3ccc(-c4cccc5sc6ccccc6c45)cc3)c3ccc4c(c3)sc3ccccc34)cc12 ; c1ccc2c(c1)ccc1ccc(N(c3ccc(-c4cccc5sc6ccccc6c45)cc3)c3ccc4sc5ccccc5c4c3)cc12 ; c1ccc2c(c1)ccc1ccc(N(c3ccc(-c4cccc5sc6ccccc6c45)cc3)c3cccc4sc5ccccc5c34)cc12] 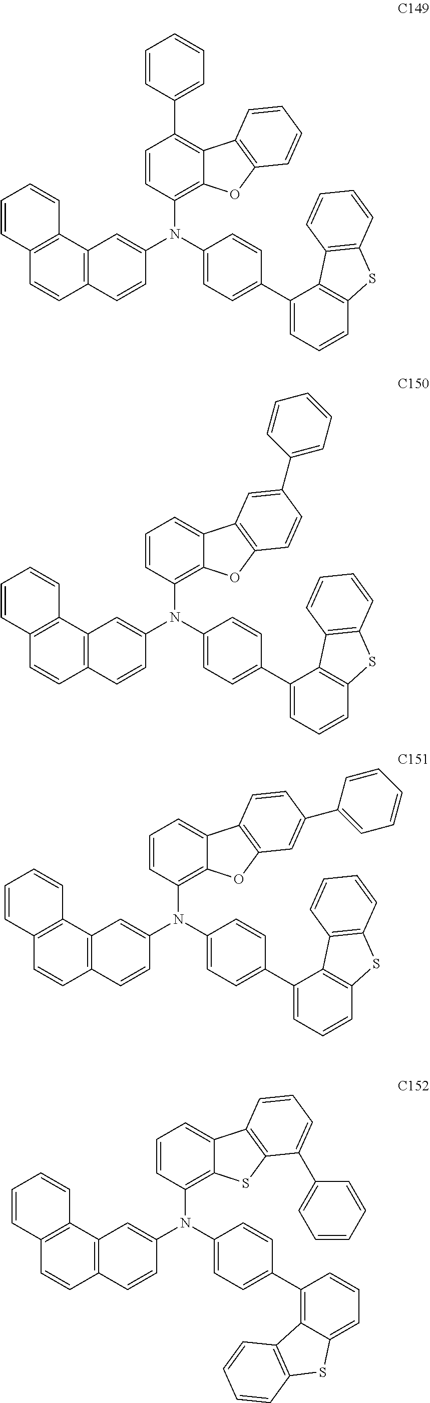 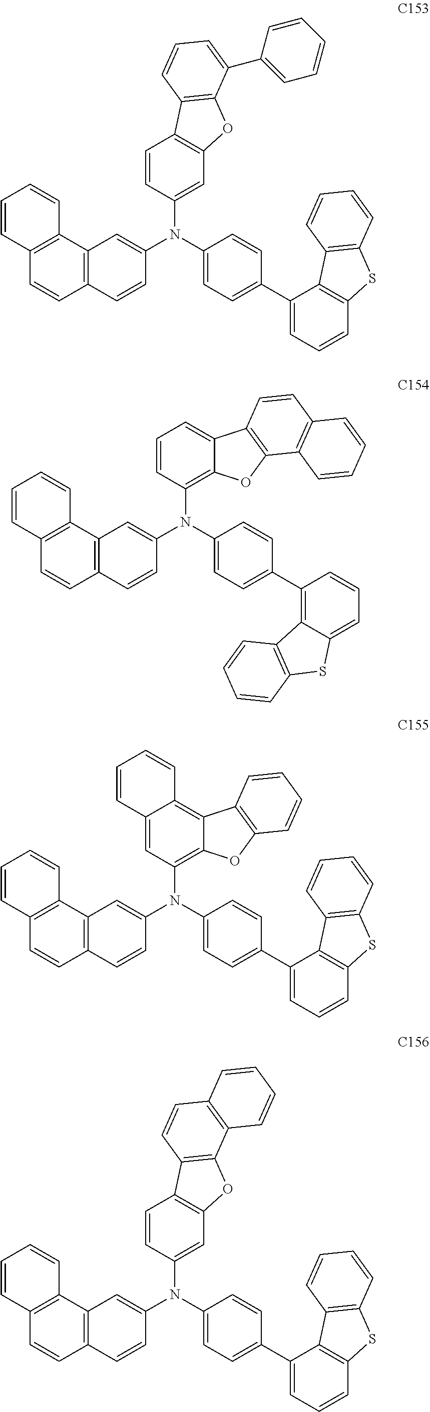 [SMILES: c1ccc(-c2cccc3c2oc2cc(N(c4ccc(-c5cccc6sc7ccccc7c56)cc4)c4ccc5ccc6ccccc6c5c4)ccc23)cc1 ; c1ccc2c(c1)ccc1ccc(N(c3ccc(-c4cccc5sc6ccccc6c45)cc3)c3cccc4c3oc3c5ccccc5ccc43)cc12 ; c1ccc2c(c1)ccc1ccc(N(c3ccc(-c4cccc5sc6ccccc6c45)cc3)c3cc4ccccc4c4c3oc3ccccc34)cc12 ; c1ccc2c(c1)ccc1ccc(N(c3ccc(-c4cccc5sc6ccccc6c45)cc3)c3ccc4c(c3)oc3c5ccccc5ccc43)cc12] 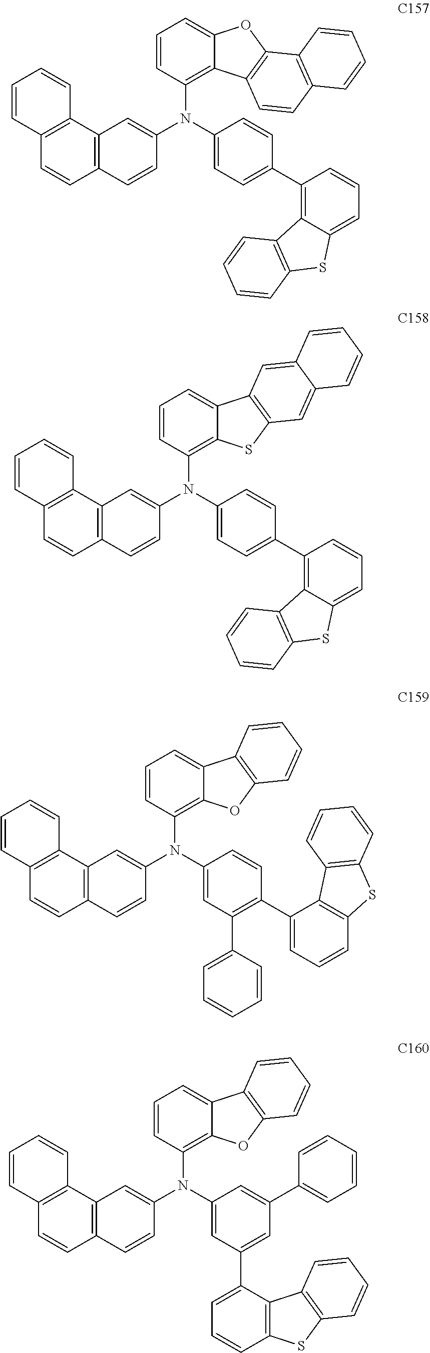 [SMILES: c1ccc2c(c1)ccc1ccc(N(c3ccc(-c4cccc5sc6ccccc6c45)cc3)c3cccc4oc5c6ccccc6ccc5c34)cc12 ; c1ccc2cc3c(cc2c1)sc1c(N(c2ccc(-c4cccc5sc6ccccc6c45)cc2)c2ccc4ccc5ccccc5c4c2)cccc13 ; c1ccc(-c2cc(N(c3ccc4ccc5ccccc5c4c3)c3cccc4c3oc3ccccc34)ccc2-c2cccc3sc4ccccc4c23)cc1 ; c1ccc(-c2cc(-c3cccc4sc5ccccc5c34)cc(N(c3ccc4ccc5ccccc5c4c3)c3cccc4c3oc3ccccc34)c2)cc1] 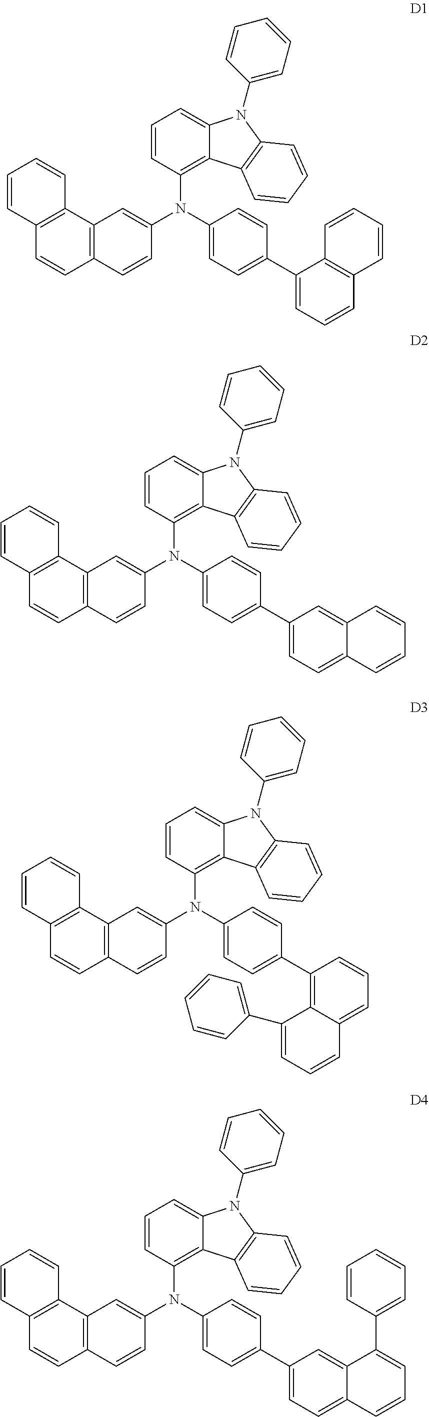 [SMILES: c1ccc(-n2c3ccccc3c3c(N(c4ccc(-c5cccc6ccccc56)cc4)c4ccc5ccc6ccccc6c5c4)cccc32)cc1 ; c1ccc(-n2c3ccccc3c3c(N(c4ccc(-c5ccc6ccccc6c5)cc4)c4ccc5ccc6ccccc6c5c4)cccc32)cc1 ; c1ccc(-c2cccc3cccc(-c4ccc(N(c5ccc6ccc7ccccc7c6c5)c5cccc6c5c5ccccc5n6-c5ccccc5)cc4)c23)cc1 ; c1ccc(-c2cccc3ccc(-c4ccc(N(c5ccc6ccc7ccccc7c6c5)c5cccc6c5c5ccccc5n6-c5ccccc5)cc4)cc23)cc1] 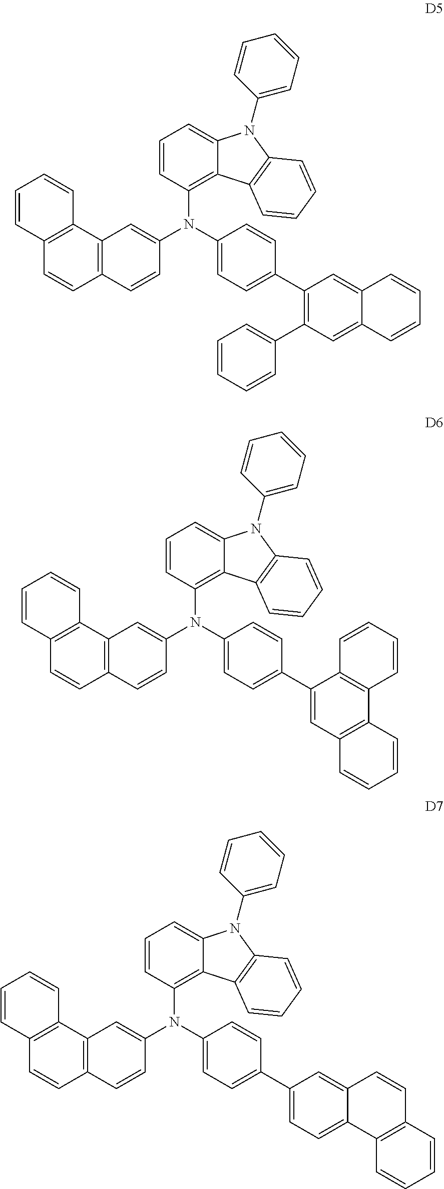 [SMILES: c1ccc(-c2cc3ccccc3cc2-c2ccc(N(c3ccc4ccc5ccccc5c4c3)c3cccc4c3c3ccccc3n4-c3ccccc3)cc2)cc1 ; c1ccc(-n2c3ccccc3c3c(N(c4ccc(-c5cc6ccccc6c6ccccc56)cc4)c4ccc5ccc6ccccc6c5c4)cccc32)cc1 ; c1ccc(-n2c3ccccc3c3c(N(c4ccc(-c5ccc6c(ccc7ccccc76)c5)cc4)c4ccc5ccc6ccccc6c5c4)cccc32)cc1] 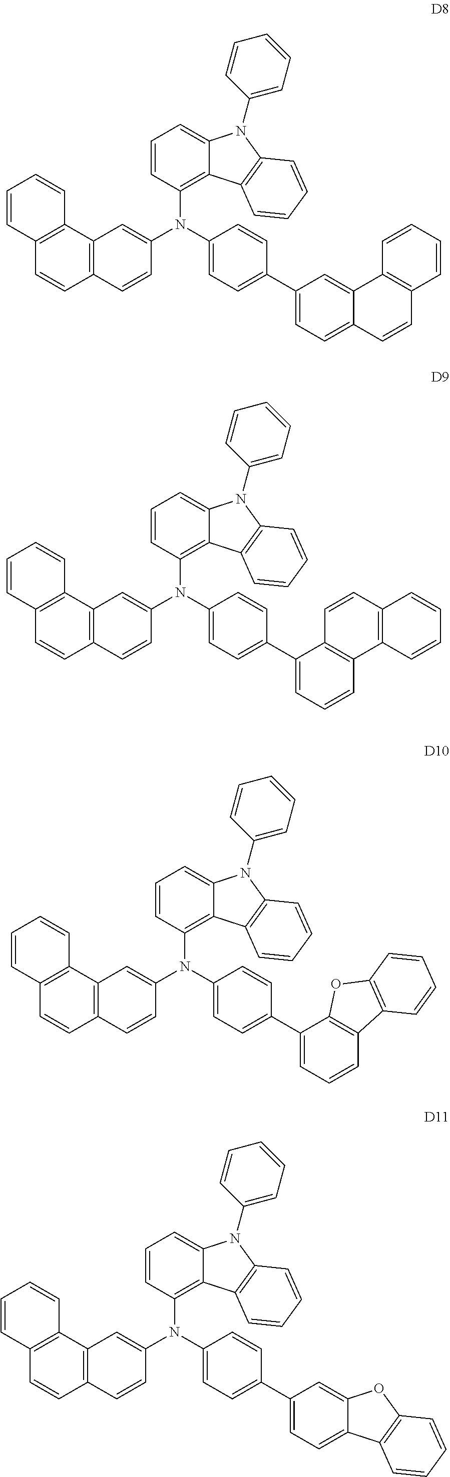 [SMILES: c1ccc(-n2c3ccccc3c3c(N(c4ccc(-c5ccc6ccc7ccccc7c6c5)cc4)c4ccc5ccc6ccccc6c5c4)cccc32)cc1 ; c1ccc(-n2c3ccccc3c3c(N(c4ccc(-c5cccc6c5ccc5ccccc56)cc4)c4ccc5ccc6ccccc6c5c4)cccc32)cc1 ; c1ccc(-n2c3ccccc3c3c(N(c4ccc(-c5cccc6c5oc5ccccc56)cc4)c4ccc5ccc6ccccc6c5c4)cccc32)cc1 ; c1ccc(-n2c3ccccc3c3c(N(c4ccc(-c5ccc6c(c5)oc5ccccc56)cc4)c4ccc5ccc6ccccc6c5c4)cccc32)cc1] 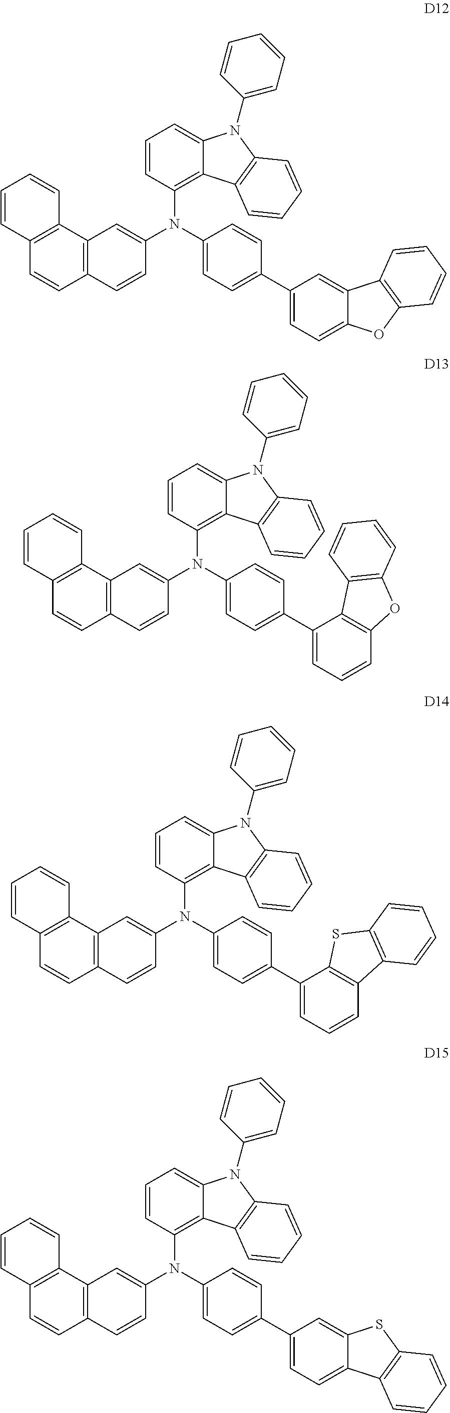 [SMILES: c1ccc(-n2c3ccccc3c3c(N(c4ccc(-c5ccc6oc7ccccc7c6c5)cc4)c4ccc5ccc6ccccc6c5c4)cccc32)cc1 ; c1ccc(-n2c3ccccc3c3c(N(c4ccc(-c5cccc6oc7ccccc7c56)cc4)c4ccc5ccc6ccccc6c5c4)cccc32)cc1 ; c1ccc(-n2c3ccccc3c3c(N(c4ccc(-c5cccc6c5sc5ccccc56)cc4)c4ccc5ccc6ccccc6c5c4)cccc32)cc1 ; c1ccc(-n2c3ccccc3c3c(N(c4ccc(-c5ccc6c(c5)sc5ccccc56)cc4)c4ccc5ccc6ccccc6c5c4)cccc32)cc1] 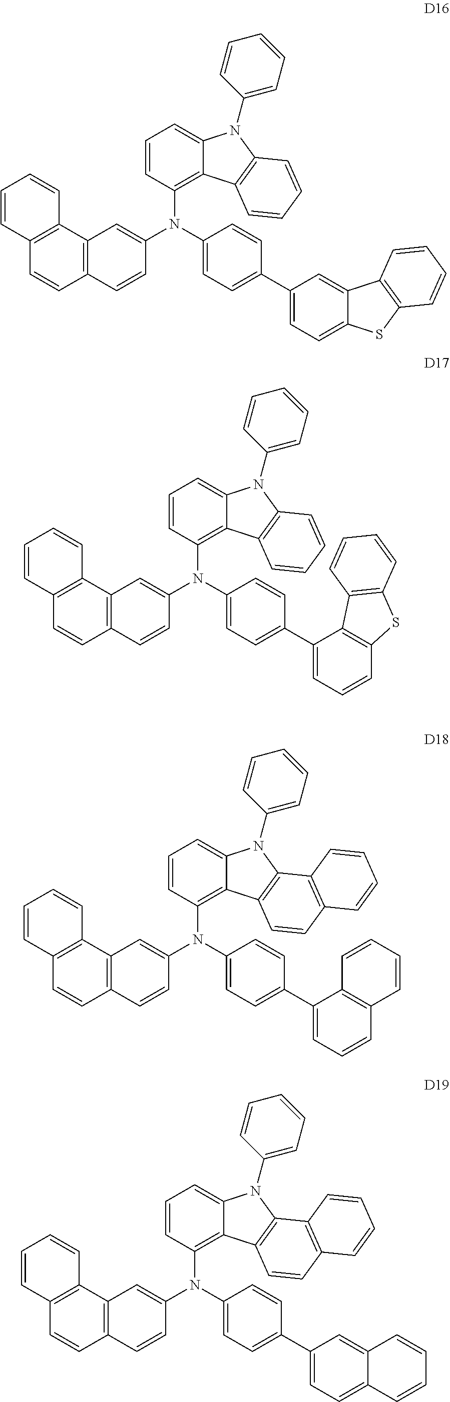 [SMILES: c1ccc(-n2c3ccccc3c3c(N(c4ccc(-c5ccc6sc7ccccc7c6c5)cc4)c4ccc5ccc6ccccc6c5c4)cccc32)cc1 ; c1ccc(-n2c3ccccc3c3c(N(c4ccc(-c5cccc6sc7ccccc7c56)cc4)c4ccc5ccc6ccccc6c5c4)cccc32)cc1 ; c1ccc(-n2c3cccc(N(c4ccc(-c5cccc6ccccc56)cc4)c4ccc5ccc6ccccc6c5c4)c3c3ccc4ccccc4c32)cc1 ; c1ccc(-n2c3cccc(N(c4ccc(-c5ccc6ccccc6c5)cc4)c4ccc5ccc6ccccc6c5c4)c3c3ccc4ccccc4c32)cc1] 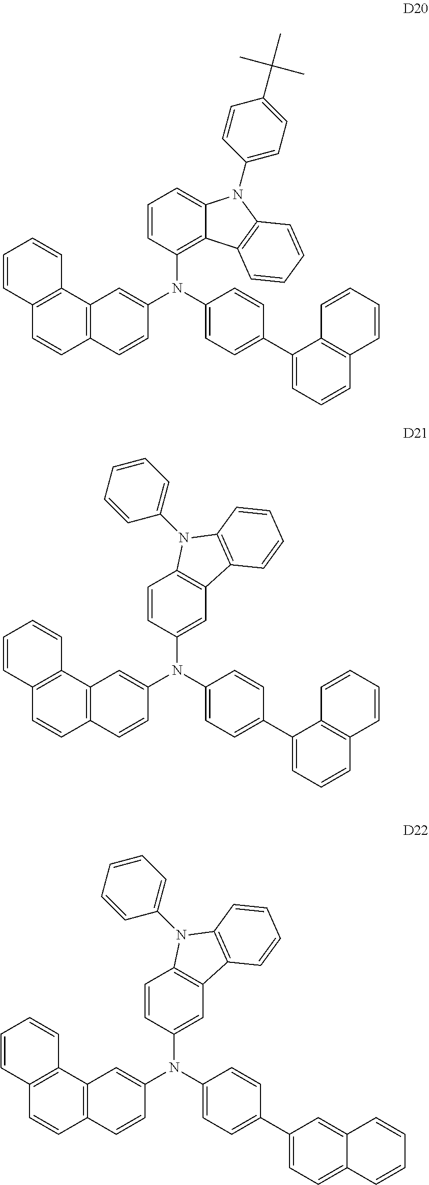 [SMILES: CC(C)(C)c1ccc(-n2c3ccccc3c3c(N(c4ccc(-c5cccc6ccccc56)cc4)c4ccc5ccc6ccccc6c5c4)cccc32)cc1 ; c1ccc(-n2c3ccccc3c3cc(N(c4ccc(-c5cccc6ccccc56)cc4)c4ccc5ccc6ccccc6c5c4)ccc32)cc1 ; c1ccc(-n2c3ccccc3c3cc(N(c4ccc(-c5ccc6ccccc6c5)cc4)c4ccc5ccc6ccccc6c5c4)ccc32)cc1] 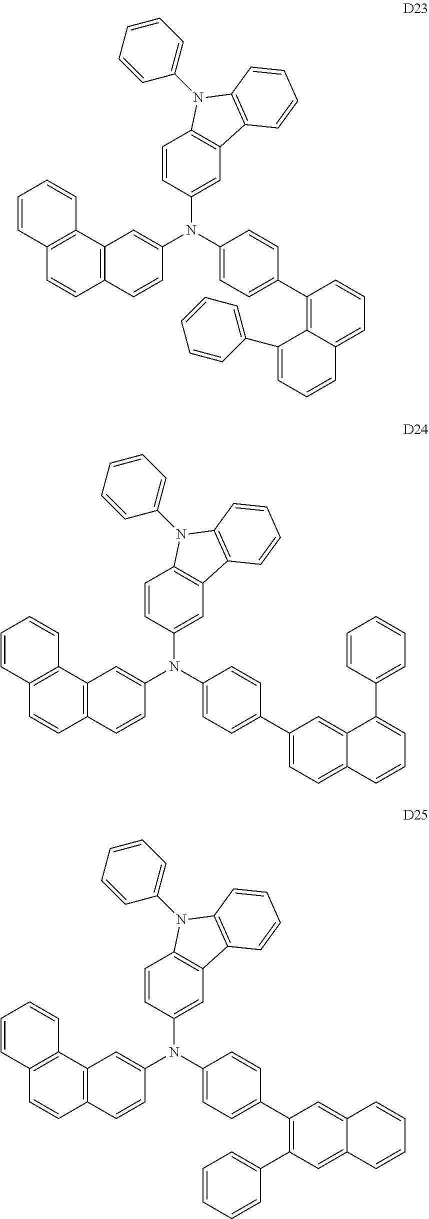 [SMILES: c1ccc(-c2cccc3cccc(-c4ccc(N(c5ccc6ccc7ccccc7c6c5)c5ccc6c(c5)c5ccccc5n6-c5ccccc5)cc4)c23)cc1 ; c1ccc(-c2cccc3ccc(-c4ccc(N(c5ccc6ccc7ccccc7c6c5)c5ccc6c(c5)c5ccccc5n6-c5ccccc5)cc4)cc23)cc1 ; c1ccc(-c2cc3ccccc3cc2-c2ccc(N(c3ccc4ccc5ccccc5c4c3)c3ccc4c(c3)c3ccccc3n4-c3ccccc3)cc2)cc1] 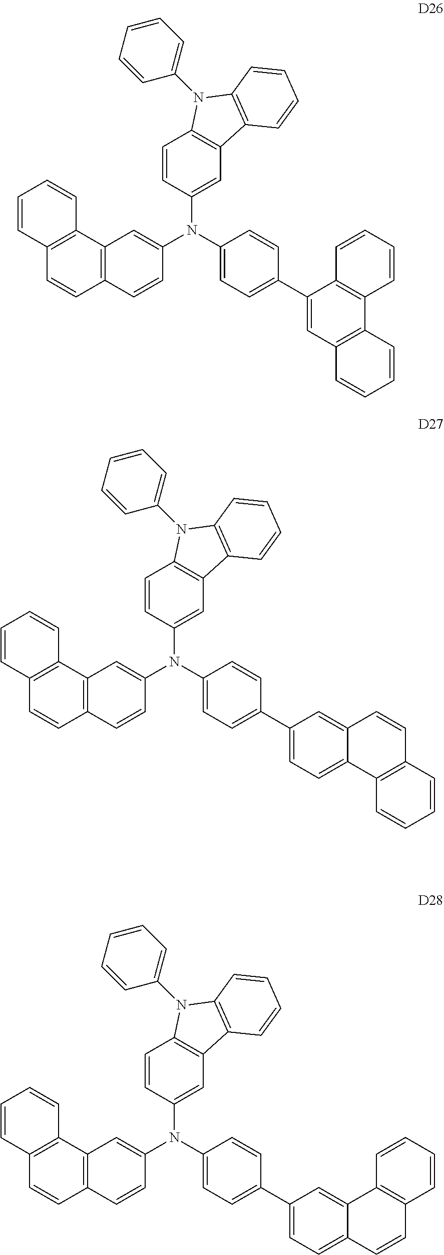 [SMILES: c1ccc(-n2c3ccccc3c3cc(N(c4ccc(-c5cc6ccccc6c6ccccc56)cc4)c4ccc5ccc6ccccc6c5c4)ccc32)cc1 ; c1ccc(-n2c3ccccc3c3cc(N(c4ccc(-c5ccc6c(ccc7ccccc76)c5)cc4)c4ccc5ccc6ccccc6c5c4)ccc32)cc1 ; c1ccc(-n2c3ccccc3c3cc(N(c4ccc(-c5ccc6ccc7ccccc7c6c5)cc4)c4ccc5ccc6ccccc6c5c4)ccc32)cc1] 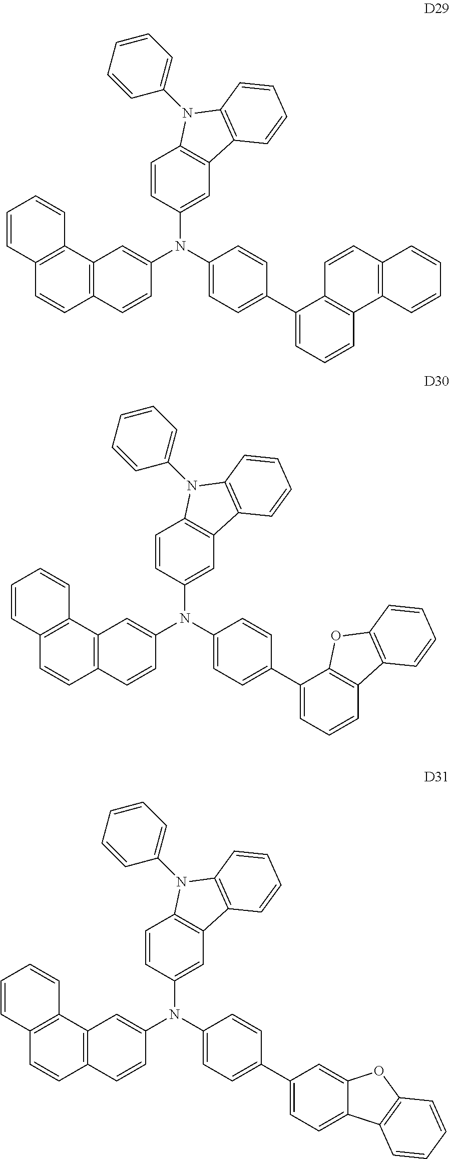 [SMILES: c1ccc(-n2c3ccccc3c3cc(N(c4ccc(-c5cccc6c5ccc5ccccc56)cc4)c4ccc5ccc6ccccc6c5c4)ccc32)cc1 ; c1ccc(-n2c3ccccc3c3cc(N(c4ccc(-c5cccc6c5oc5ccccc56)cc4)c4ccc5ccc6ccccc6c5c4)ccc32)cc1 ; c1ccc(-n2c3ccccc3c3cc(N(c4ccc(-c5ccc6c(c5)oc5ccccc56)cc4)c4ccc5ccc6ccccc6c5c4)ccc32)cc1] 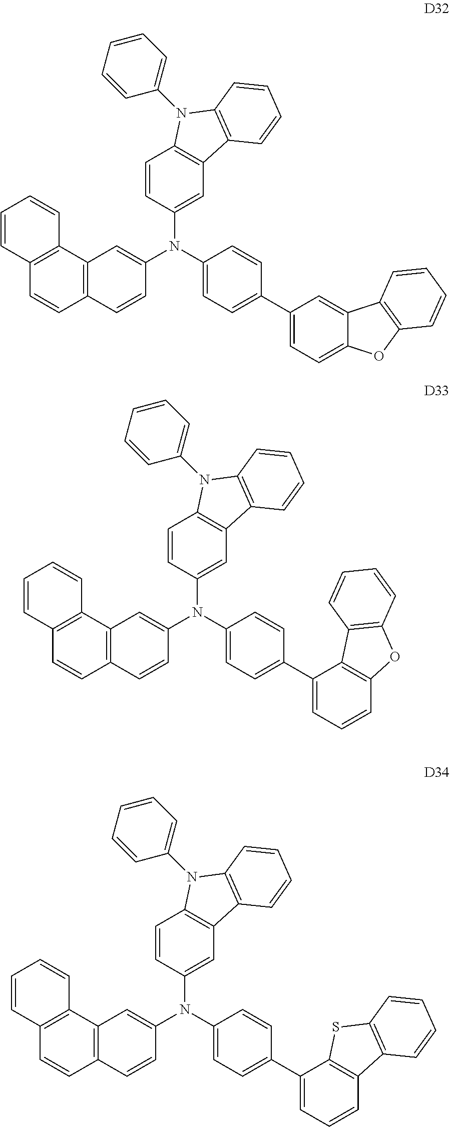 [SMILES: c1ccc(-n2c3ccccc3c3cc(N(c4ccc(-c5ccc6oc7ccccc7c6c5)cc4)c4ccc5ccc6ccccc6c5c4)ccc32)cc1 ; c1ccc(-n2c3ccccc3c3cc(N(c4ccc(-c5cccc6oc7ccccc7c56)cc4)c4ccc5ccc6ccccc6c5c4)ccc32)cc1 ; c1ccc(-n2c3ccccc3c3cc(N(c4ccc(-c5cccc6c5sc5ccccc56)cc4)c4ccc5ccc6ccccc6c5c4)ccc32)cc1] 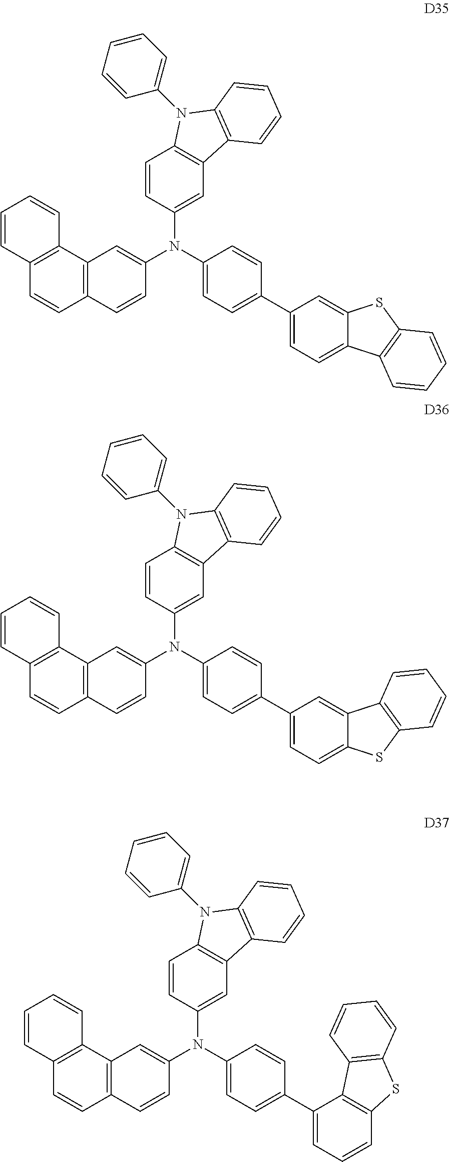 [SMILES: c1ccc(-n2c3ccccc3c3cc(N(c4ccc(-c5ccc6c(c5)sc5ccccc56)cc4)c4ccc5ccc6ccccc6c5c4)ccc32)cc1 ; c1ccc(-n2c3ccccc3c3cc(N(c4ccc(-c5ccc6sc7ccccc7c6c5)cc4)c4ccc5ccc6ccccc6c5c4)ccc32)cc1 ; c1ccc(-n2c3ccccc3c3cc(N(c4ccc(-c5cccc6sc7ccccc7c56)cc4)c4ccc5ccc6ccccc6c5c4)ccc32)cc1] 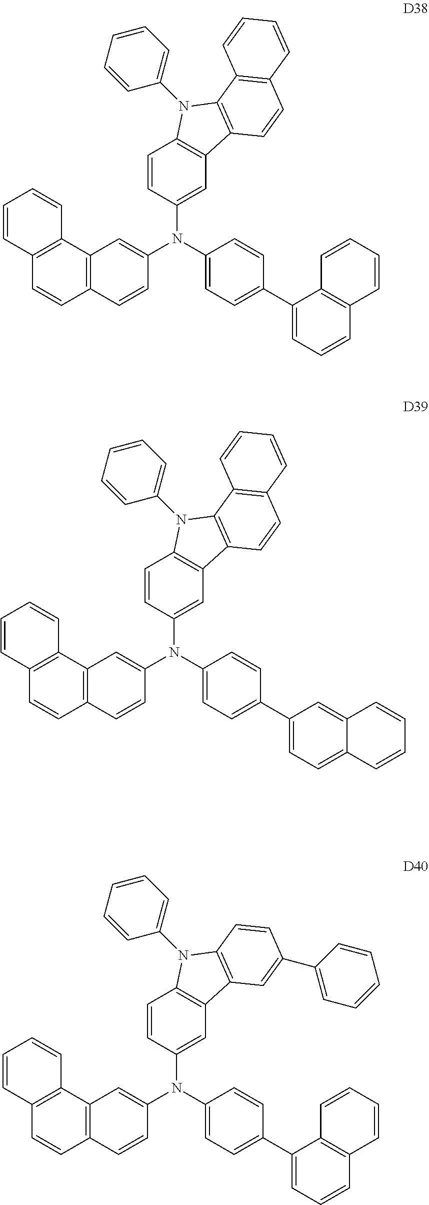 [SMILES: c1ccc(-n2c3ccc(N(c4ccc(-c5cccc6ccccc56)cc4)c4ccc5ccc6ccccc6c5c4)cc3c3ccc4ccccc4c32)cc1 ; c1ccc(-n2c3ccc(N(c4ccc(-c5ccc6ccccc6c5)cc4)c4ccc5ccc6ccccc6c5c4)cc3c3ccc4ccccc4c32)cc1 ; c1ccc(-c2ccc3c(c2)c2cc(N(c4ccc(-c5cccc6ccccc56)cc4)c4ccc5ccc6ccccc6c5c4)ccc2n3-c2ccccc2)cc1] 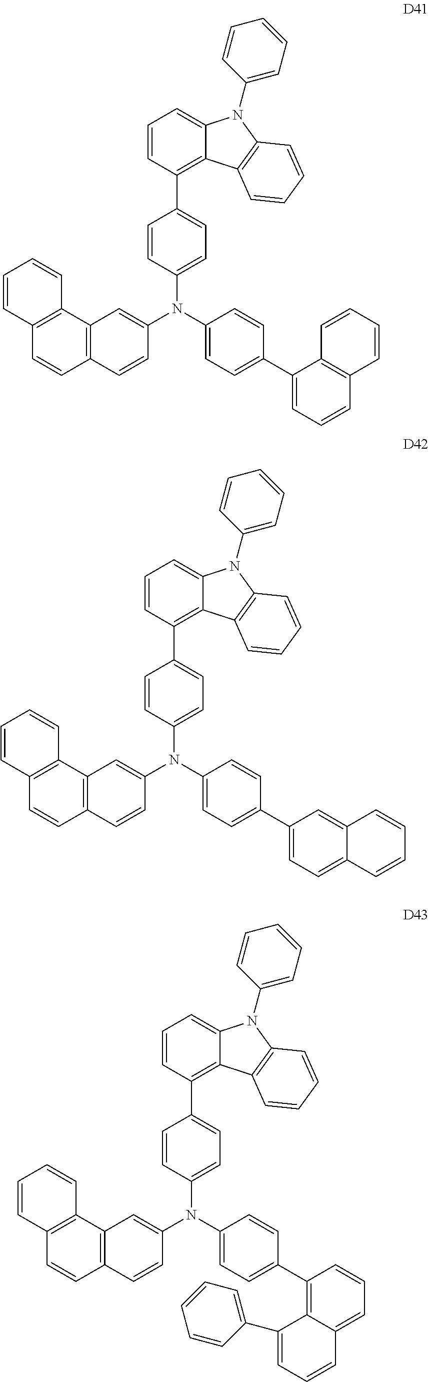 [SMILES: c1ccc(-n2c3ccccc3c3c(-c4ccc(N(c5ccc(-c6cccc7ccccc67)cc5)c5ccc6ccc7ccccc7c6c5)cc4)cccc32)cc1 ; c1ccc(-n2c3ccccc3c3c(-c4ccc(N(c5ccc(-c6ccc7ccccc7c6)cc5)c5ccc6ccc7ccccc7c6c5)cc4)cccc32)cc1 ; c1ccc(-c2cccc3cccc(-c4ccc(N(c5ccc(-c6cccc7c6c6ccccc6n7-c6ccccc6)cc5)c5ccc6ccc7ccccc7c6c5)cc4)c23)cc1] 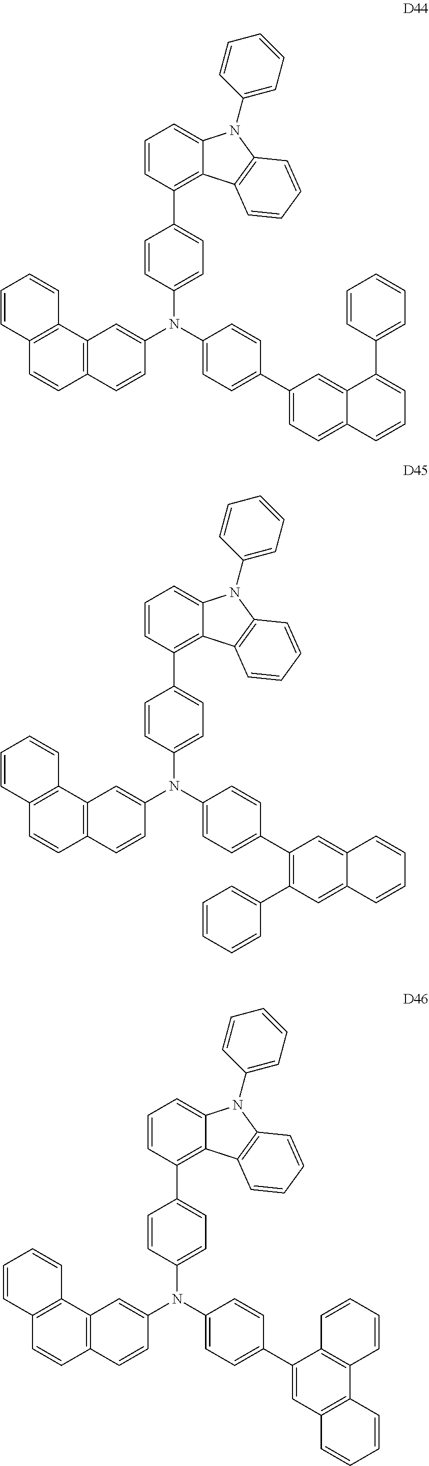 [SMILES: c1ccc(-c2cccc3ccc(-c4ccc(N(c5ccc(-c6cccc7c6c6ccccc6n7-c6ccccc6)cc5)c5ccc6ccc7ccccc7c6c5)cc4)cc23)cc1 ; c1ccc(-c2cc3ccccc3cc2-c2ccc(N(c3ccc(-c4cccc5c4c4ccccc4n5-c4ccccc4)cc3)c3ccc4ccc5ccccc5c4c3)cc2)cc1 ; c1ccc(-n2c3ccccc3c3c(-c4ccc(N(c5ccc(-c6cc7ccccc7c7ccccc67)cc5)c5ccc6ccc7ccccc7c6c5)cc4)cccc32)cc1] 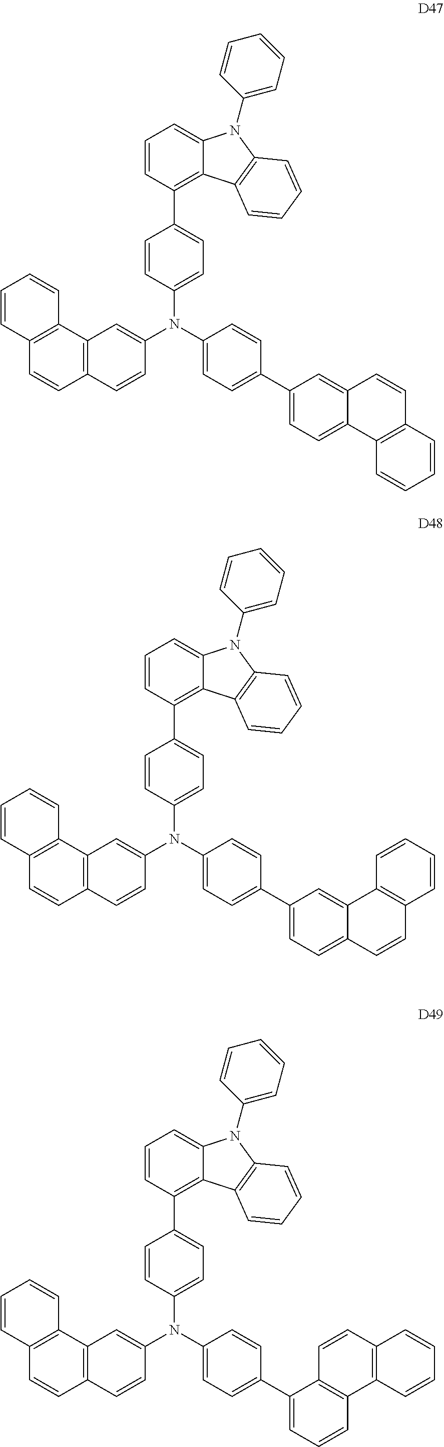 [SMILES: c1ccc(-n2c3ccccc3c3c(-c4ccc(N(c5ccc(-c6ccc7c(ccc8ccccc87)c6)cc5)c5ccc6ccc7ccccc7c6c5)cc4)cccc32)cc1 ; c1ccc(-n2c3ccccc3c3c(-c4ccc(N(c5ccc(-c6ccc7ccc8ccccc8c7c6)cc5)c5ccc6ccc7ccccc7c6c5)cc4)cccc32)cc1 ; c1ccc(-n2c3ccccc3c3c(-c4ccc(N(c5ccc(-c6cccc7c6ccc6ccccc67)cc5)c5ccc6ccc7ccccc7c6c5)cc4)cccc32)cc1] 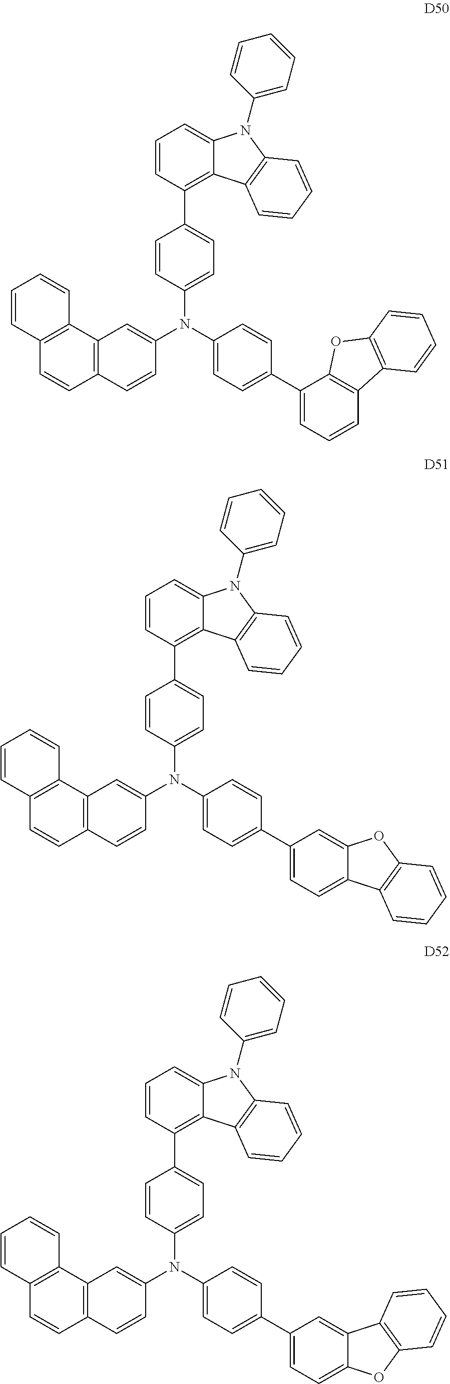 [SMILES: c1ccc(-n2c3ccccc3c3c(-c4ccc(N(c5ccc(-c6cccc7c6oc6ccccc67)cc5)c5ccc6ccc7ccccc7c6c5)cc4)cccc32)cc1 ; c1ccc(-n2c3ccccc3c3c(-c4ccc(N(c5ccc(-c6ccc7c(c6)oc6ccccc67)cc5)c5ccc6ccc7ccccc7c6c5)cc4)cccc32)cc1 ; c1ccc(-n2c3ccccc3c3c(-c4ccc(N(c5ccc(-c6ccc7oc8ccccc8c7c6)cc5)c5ccc6ccc7ccccc7c6c5)cc4)cccc32)cc1] 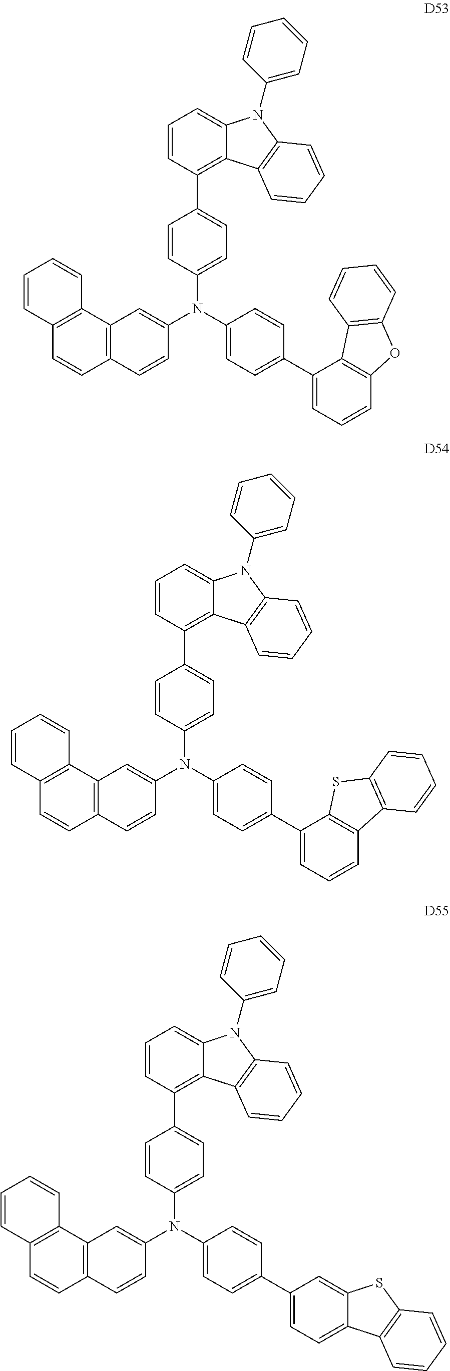 [SMILES: c1ccc(-n2c3ccccc3c3c(-c4ccc(N(c5ccc(-c6cccc7oc8ccccc8c67)cc5)c5ccc6ccc7ccccc7c6c5)cc4)cccc32)cc1 ; c1ccc(-n2c3ccccc3c3c(-c4ccc(N(c5ccc(-c6cccc7c6sc6ccccc67)cc5)c5ccc6ccc7ccccc7c6c5)cc4)cccc32)cc1 ; c1ccc(-n2c3ccccc3c3c(-c4ccc(N(c5ccc(-c6ccc7c(c6)sc6ccccc67)cc5)c5ccc6ccc7ccccc7c6c5)cc4)cccc32)cc1] 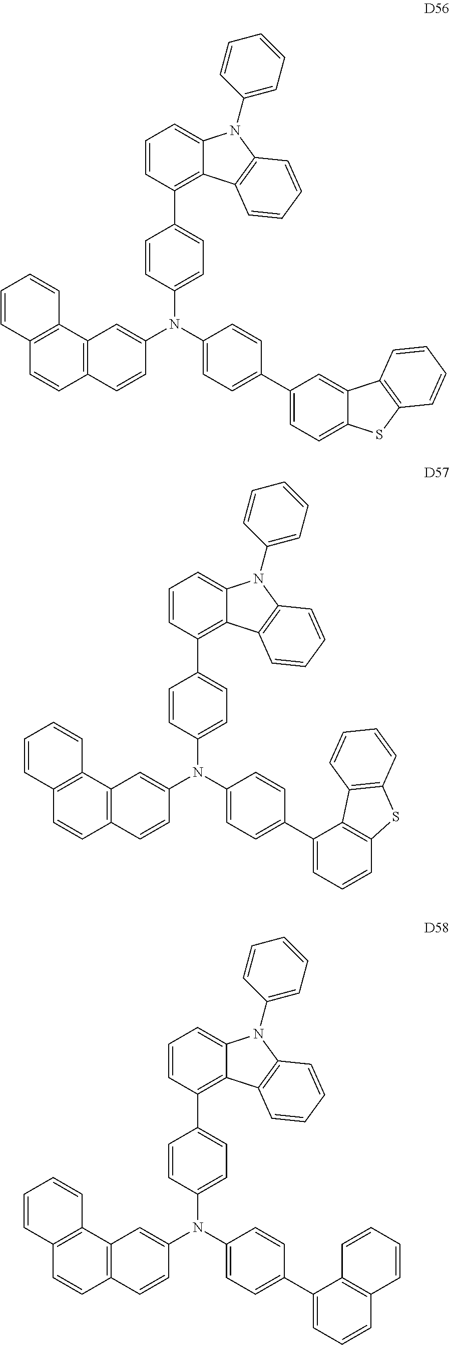 [SMILES: c1ccc(-n2c3ccccc3c3c(-c4ccc(N(c5ccc(-c6ccc7sc8ccccc8c7c6)cc5)c5ccc6ccc7ccccc7c6c5)cc4)cccc32)cc1 ; c1ccc(-n2c3ccccc3c3c(-c4ccc(N(c5ccc(-c6cccc7sc8ccccc8c67)cc5)c5ccc6ccc7ccccc7c6c5)cc4)cccc32)cc1 ; c1ccc(-n2c3ccccc3c3c(-c4ccc(N(c5ccc(-c6cccc7ccccc67)cc5)c5ccc6ccc7ccccc7c6c5)cc4)cccc32)cc1] 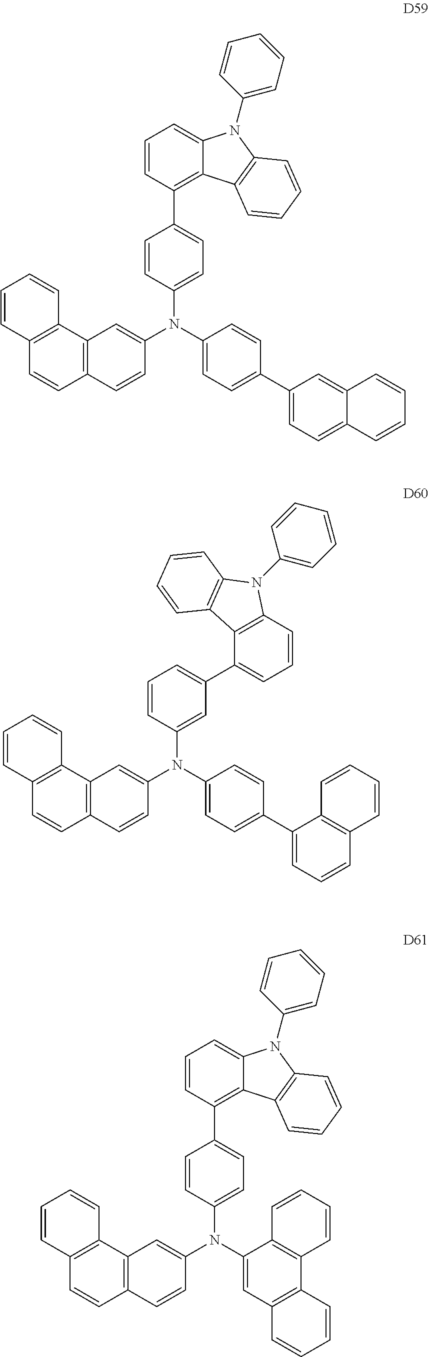 [SMILES: c1ccc(-n2c3ccccc3c3c(-c4ccc(N(c5ccc(-c6ccc7ccccc7c6)cc5)c5ccc6ccc7ccccc7c6c5)cc4)cccc32)cc1 ; c1ccc(-n2c3ccccc3c3c(-c4cccc(N(c5ccc(-c6cccc7ccccc67)cc5)c5ccc6ccc7ccccc7c6c5)c4)cccc32)cc1 ; c1ccc(-n2c3ccccc3c3c(-c4ccc(N(c5ccc6ccc7ccccc7c6c5)c5cc6ccccc6c6ccccc56)cc4)cccc32)cc1] 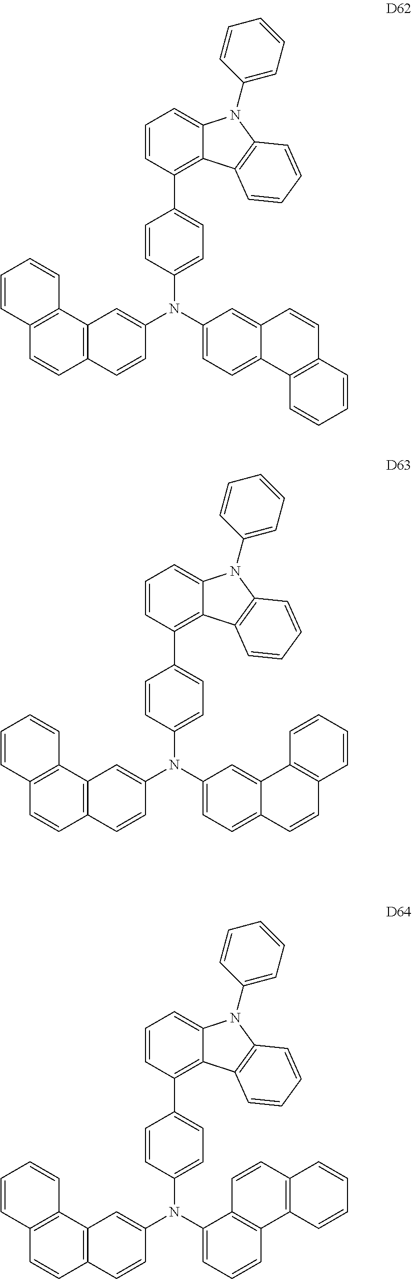 [SMILES: c1ccc(-n2c3ccccc3c3c(-c4ccc(N(c5ccc6c(ccc7ccccc76)c5)c5ccc6ccc7ccccc7c6c5)cc4)cccc32)cc1 ; c1ccc(-n2c3ccccc3c3c(-c4ccc(N(c5ccc6ccc7ccccc7c6c5)c5ccc6ccc7ccccc7c6c5)cc4)cccc32)cc1 ; c1ccc(-n2c3ccccc3c3c(-c4ccc(N(c5ccc6ccc7ccccc7c6c5)c5cccc6c5ccc5ccccc56)cc4)cccc32)cc1] 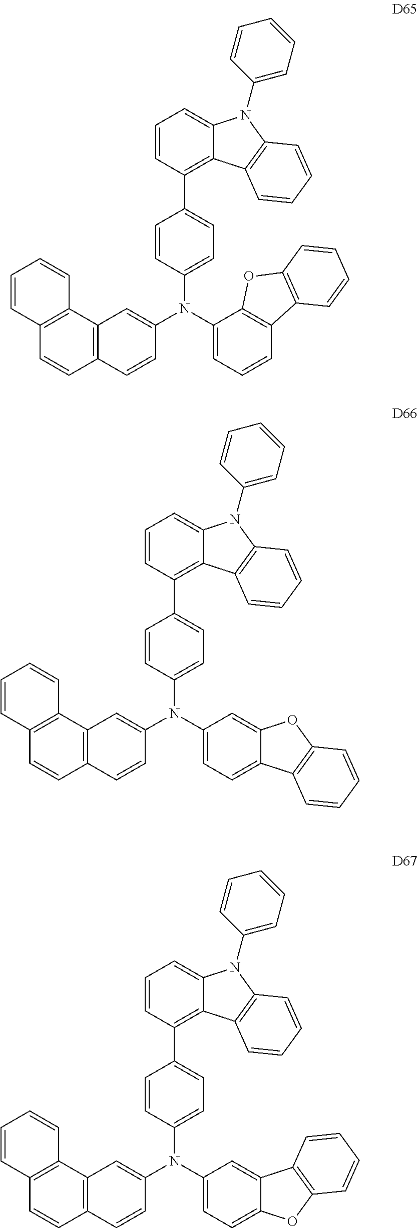 [SMILES: c1ccc(-n2c3ccccc3c3c(-c4ccc(N(c5ccc6ccc7ccccc7c6c5)c5cccc6c5oc5ccccc56)cc4)cccc32)cc1 ; c1ccc(-n2c3ccccc3c3c(-c4ccc(N(c5ccc6c(c5)oc5ccccc56)c5ccc6ccc7ccccc7c6c5)cc4)cccc32)cc1 ; c1ccc(-n2c3ccccc3c3c(-c4ccc(N(c5ccc6ccc7ccccc7c6c5)c5ccc6oc7ccccc7c6c5)cc4)cccc32)cc1] 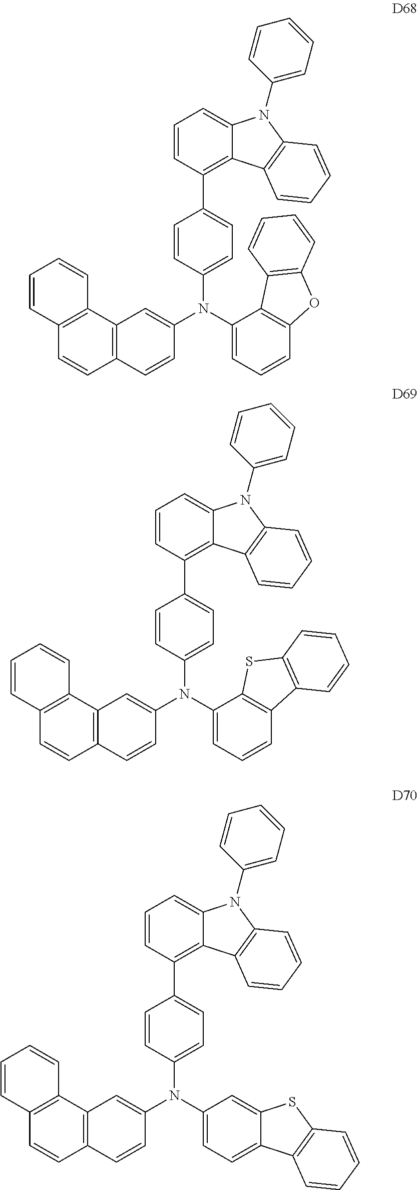 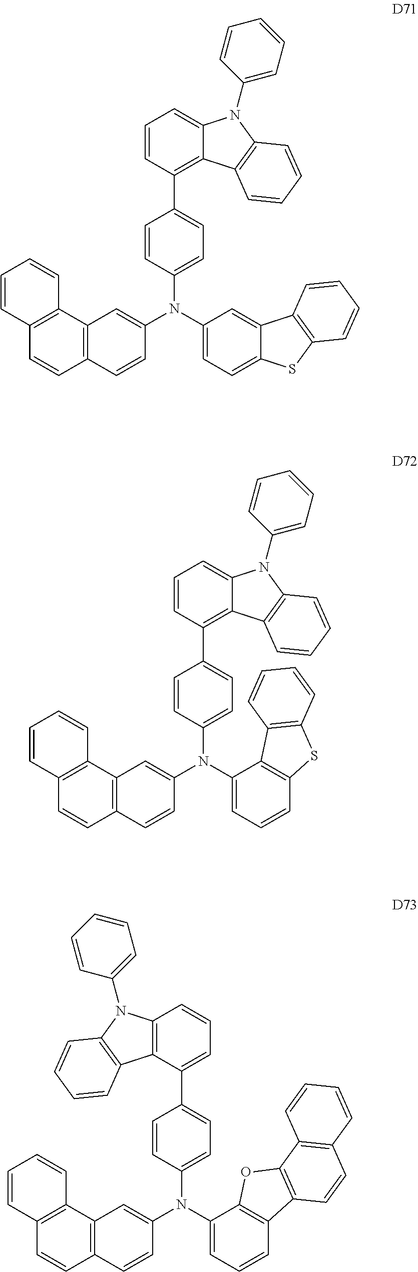 [SMILES: c1ccc(-n2c3ccccc3c3c(-c4ccc(N(c5ccc6ccc7ccccc7c6c5)c5ccc6sc7ccccc7c6c5)cc4)cccc32)cc1 ; c1ccc(-n2c3ccccc3c3c(-c4ccc(N(c5ccc6ccc7ccccc7c6c5)c5cccc6sc7ccccc7c56)cc4)cccc32)cc1 ; c1ccc(-n2c3ccccc3c3c(-c4ccc(N(c5ccc6ccc7ccccc7c6c5)c5cccc6c5oc5c7ccccc7ccc65)cc4)cccc32)cc1] 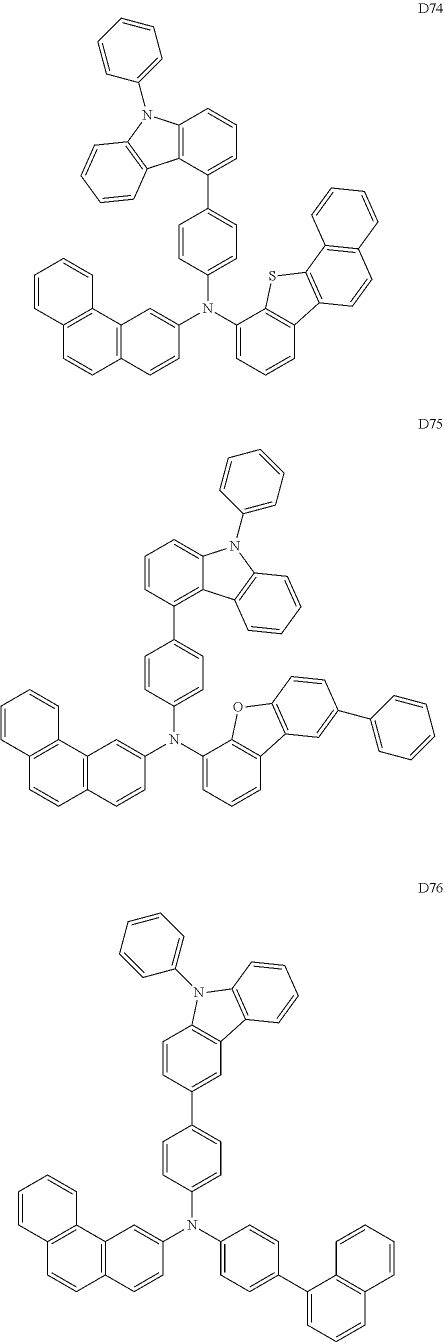 [SMILES: c1ccc(-n2c3ccccc3c3c(-c4ccc(N(c5ccc6ccc7ccccc7c6c5)c5cccc6c5sc5c7ccccc7ccc65)cc4)cccc32)cc1 ; c1ccc(-c2ccc3oc4c(N(c5ccc(-c6cccc7c6c6ccccc6n7-c6ccccc6)cc5)c5ccc6ccc7ccccc7c6c5)cccc4c3c2)cc1 ; c1ccc(-n2c3ccccc3c3cc(-c4ccc(N(c5ccc(-c6cccc7ccccc67)cc5)c5ccc6ccc7ccccc7c6c5)cc4)ccc32)cc1] 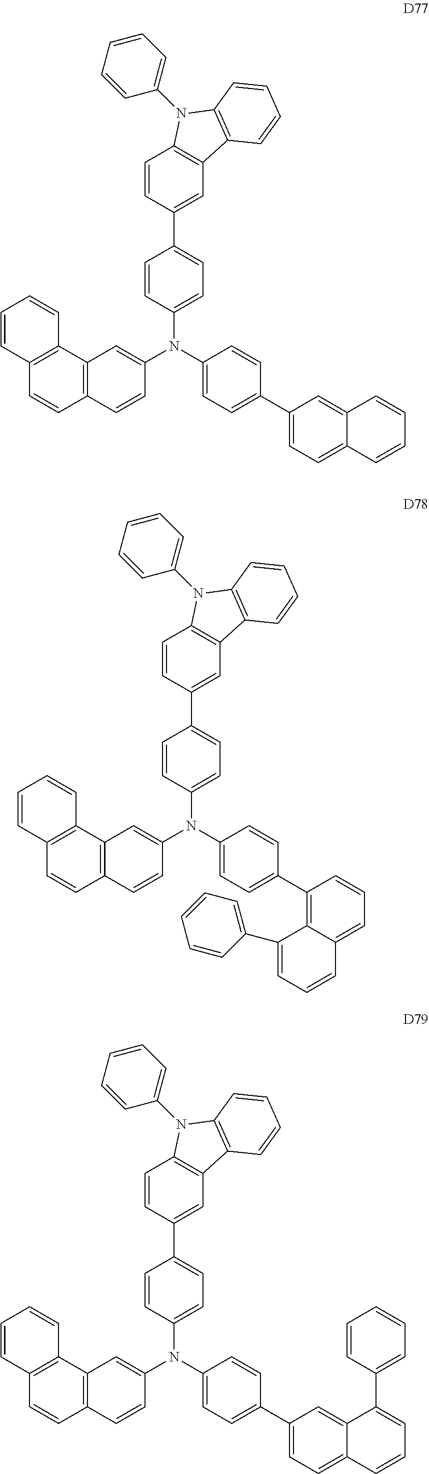 [SMILES: c1ccc(-n2c3ccccc3c3cc(-c4ccc(N(c5ccc(-c6ccc7ccccc7c6)cc5)c5ccc6ccc7ccccc7c6c5)cc4)ccc32)cc1 ; c1ccc(-c2cccc3cccc(-c4ccc(N(c5ccc(-c6ccc7c(c6)c6ccccc6n7-c6ccccc6)cc5)c5ccc6ccc7ccccc7c6c5)cc4)c23)cc1 ; c1ccc(-c2cccc3ccc(-c4ccc(N(c5ccc(-c6ccc7c(c6)c6ccccc6n7-c6ccccc6)cc5)c5ccc6ccc7ccccc7c6c5)cc4)cc23)cc1] 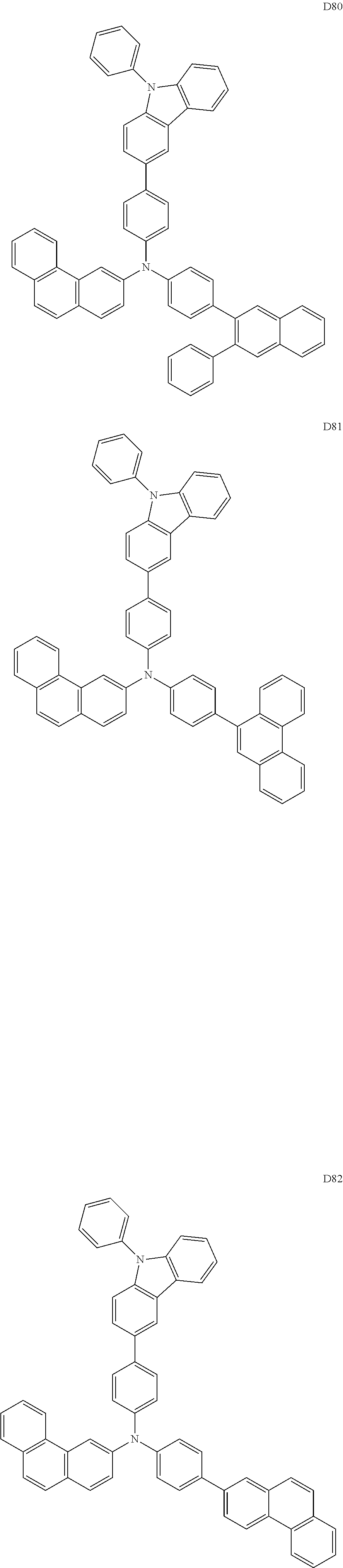 [SMILES: c1ccc(-c2cc3ccccc3cc2-c2ccc(N(c3ccc(-c4ccc5c(c4)c4ccccc4n5-c4ccccc4)cc3)c3ccc4ccc5ccccc5c4c3)cc2)cc1 ; c1ccc(-n2c3ccccc3c3cc(-c4ccc(N(c5ccc(-c6cc7ccccc7c7ccccc67)cc5)c5ccc6ccc7ccccc7c6c5)cc4)ccc32)cc1 ; c1ccc(-n2c3ccccc3c3cc(-c4ccc(N(c5ccc(-c6ccc7c(ccc8ccccc87)c6)cc5)c5ccc6ccc7ccccc7c6c5)cc4)ccc32)cc1] 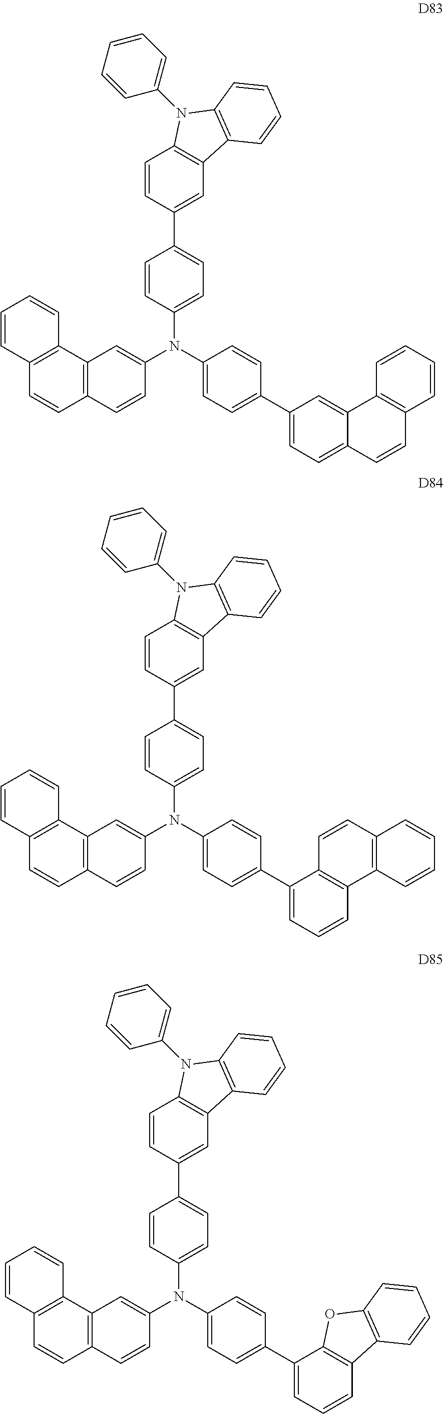 [SMILES: c1ccc(-n2c3ccccc3c3cc(-c4ccc(N(c5ccc(-c6ccc7ccc8ccccc8c7c6)cc5)c5ccc6ccc7ccccc7c6c5)cc4)ccc32)cc1 ; c1ccc(-n2c3ccccc3c3cc(-c4ccc(N(c5ccc(-c6cccc7c6ccc6ccccc67)cc5)c5ccc6ccc7ccccc7c6c5)cc4)ccc32)cc1 ; c1ccc(-n2c3ccccc3c3cc(-c4ccc(N(c5ccc(-c6cccc7c6oc6ccccc67)cc5)c5ccc6ccc7ccccc7c6c5)cc4)ccc32)cc1] 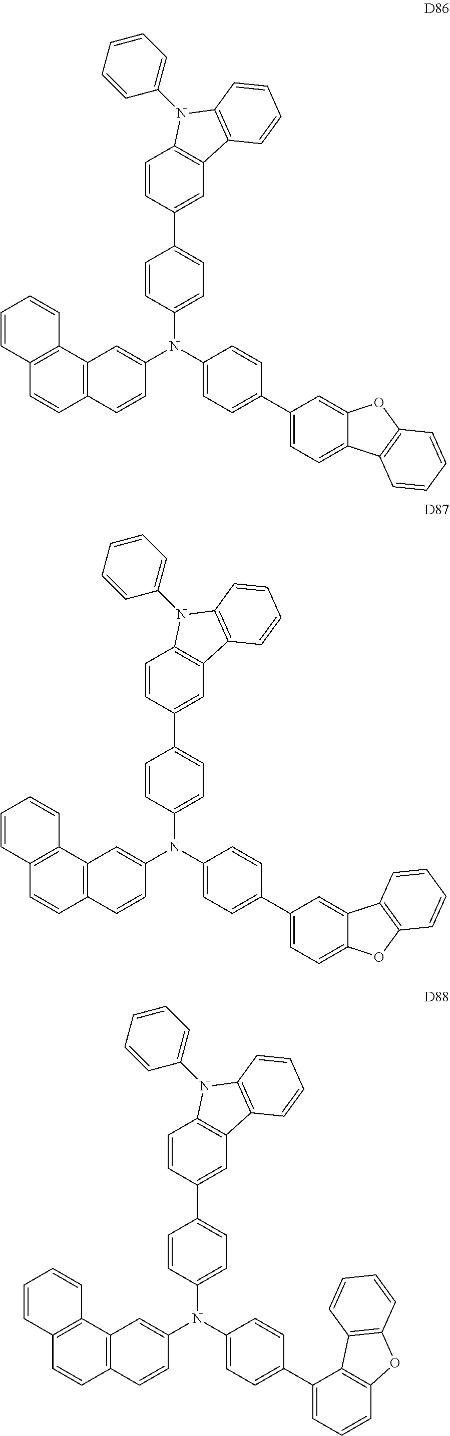 [SMILES: c1ccc(-n2c3ccccc3c3cc(-c4ccc(N(c5ccc(-c6ccc7c(c6)oc6ccccc67)cc5)c5ccc6ccc7ccccc7c6c5)cc4)ccc32)cc1 ; c1ccc(-n2c3ccccc3c3cc(-c4ccc(N(c5ccc(-c6ccc7oc8ccccc8c7c6)cc5)c5ccc6ccc7ccccc7c6c5)cc4)ccc32)cc1 ; c1ccc(-n2c3ccccc3c3cc(-c4ccc(N(c5ccc(-c6cccc7oc8ccccc8c67)cc5)c5ccc6ccc7ccccc7c6c5)cc4)ccc32)cc1] 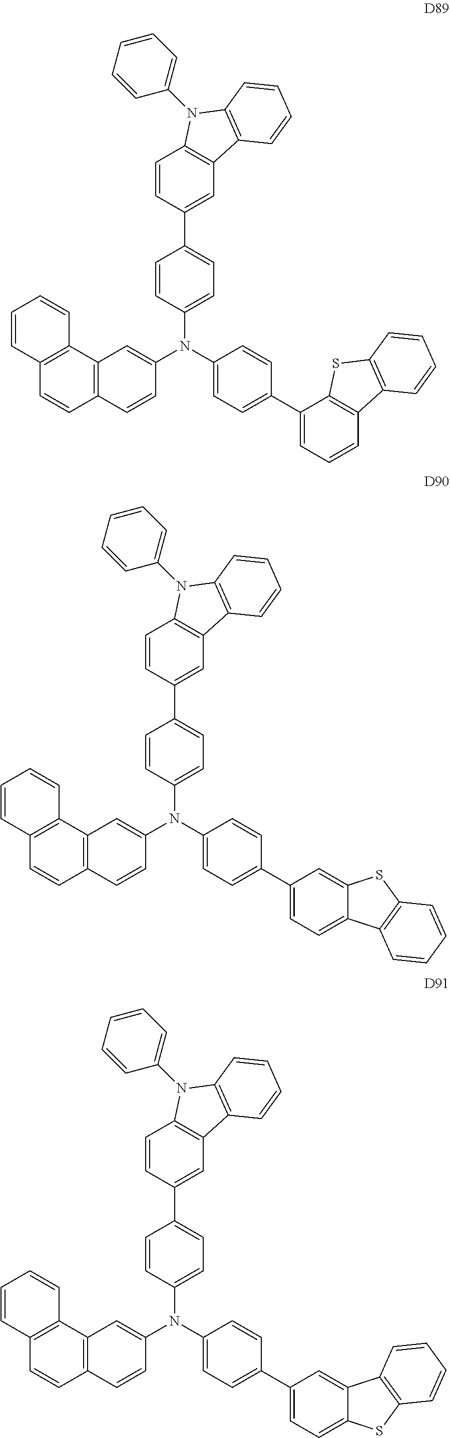 [SMILES: c1ccc(-n2c3ccccc3c3cc(-c4ccc(N(c5ccc(-c6cccc7c6sc6ccccc67)cc5)c5ccc6ccc7ccccc7c6c5)cc4)ccc32)cc1 ; c1ccc(-n2c3ccccc3c3cc(-c4ccc(N(c5ccc(-c6ccc7c(c6)sc6ccccc67)cc5)c5ccc6ccc7ccccc7c6c5)cc4)ccc32)cc1 ; c1ccc(-n2c3ccccc3c3cc(-c4ccc(N(c5ccc(-c6ccc7sc8ccccc8c7c6)cc5)c5ccc6ccc7ccccc7c6c5)cc4)ccc32)cc1] 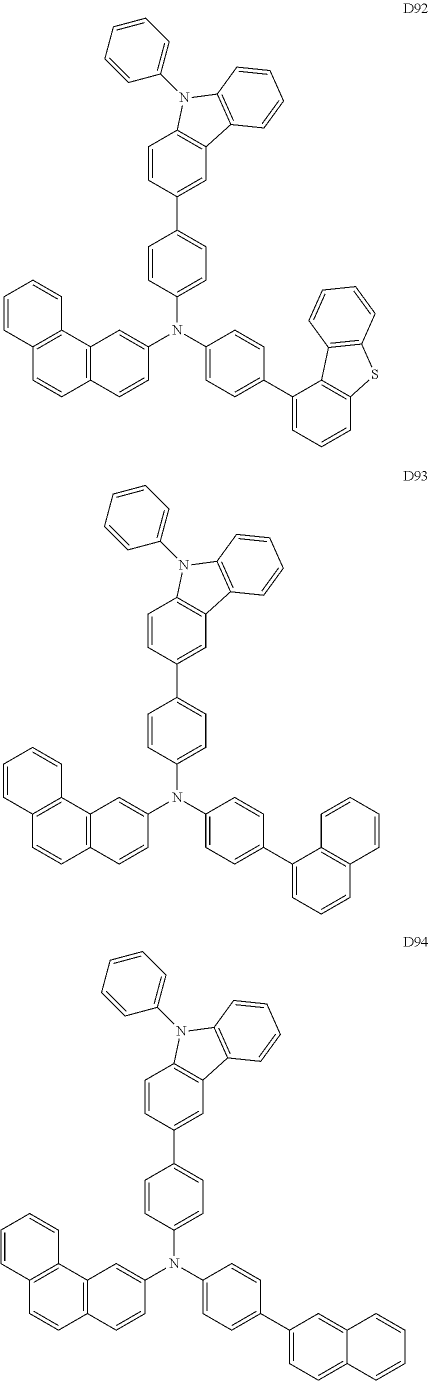 [SMILES: c1ccc(-n2c3ccccc3c3cc(-c4ccc(N(c5ccc(-c6cccc7sc8ccccc8c67)cc5)c5ccc6ccc7ccccc7c6c5)cc4)ccc32)cc1 ; c1ccc(-n2c3ccccc3c3cc(-c4ccc(N(c5ccc(-c6cccc7ccccc67)cc5)c5ccc6ccc7ccccc7c6c5)cc4)ccc32)cc1 ; c1ccc(-n2c3ccccc3c3cc(-c4ccc(N(c5ccc(-c6ccc7ccccc7c6)cc5)c5ccc6ccc7ccccc7c6c5)cc4)ccc32)cc1] 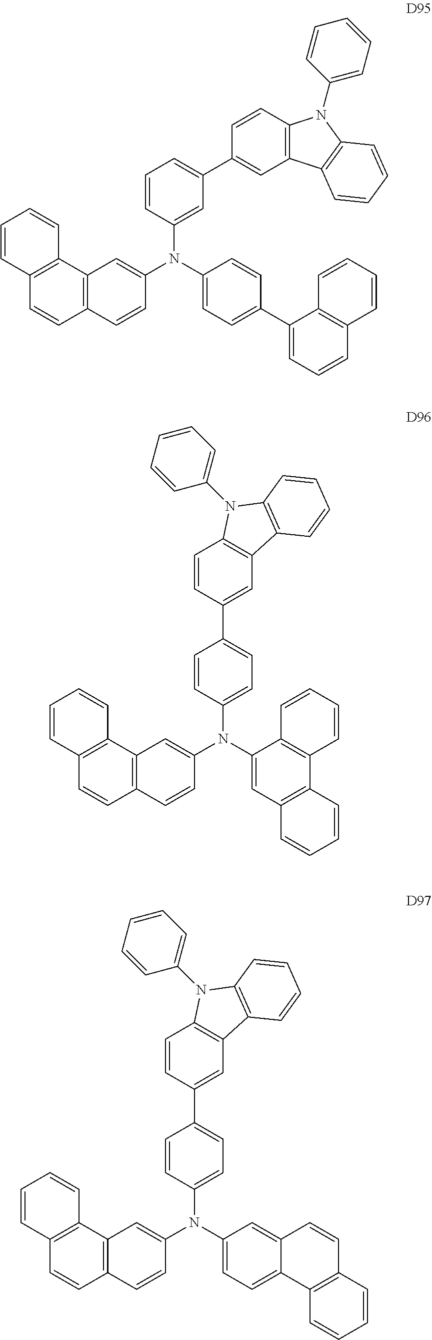 [SMILES: c1ccc(-n2c3ccccc3c3cc(-c4cccc(N(c5ccc(-c6cccc7ccccc67)cc5)c5ccc6ccc7ccccc7c6c5)c4)ccc32)cc1 ; c1ccc(-n2c3ccccc3c3cc(-c4ccc(N(c5ccc6ccc7ccccc7c6c5)c5cc6ccccc6c6ccccc56)cc4)ccc32)cc1 ; c1ccc(-n2c3ccccc3c3cc(-c4ccc(N(c5ccc6c(ccc7ccccc76)c5)c5ccc6ccc7ccccc7c6c5)cc4)ccc32)cc1] 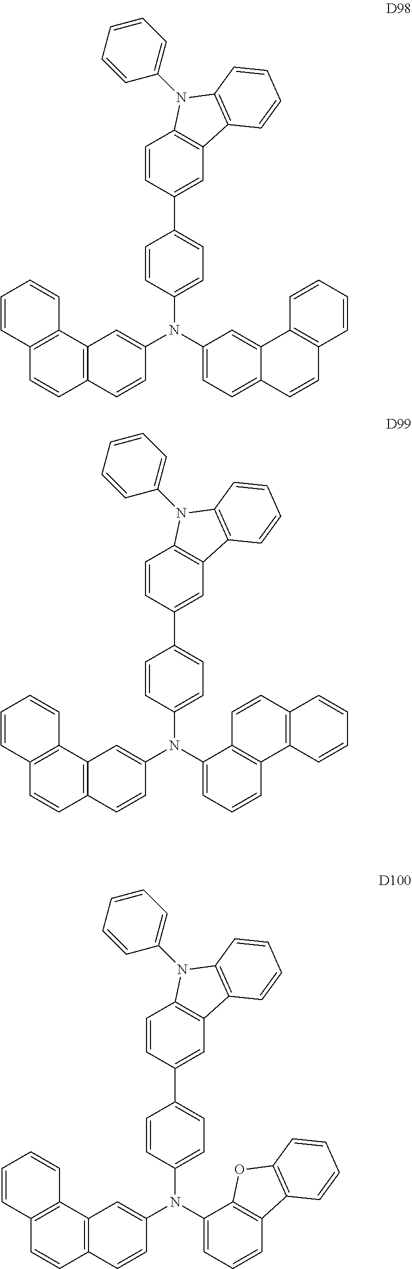 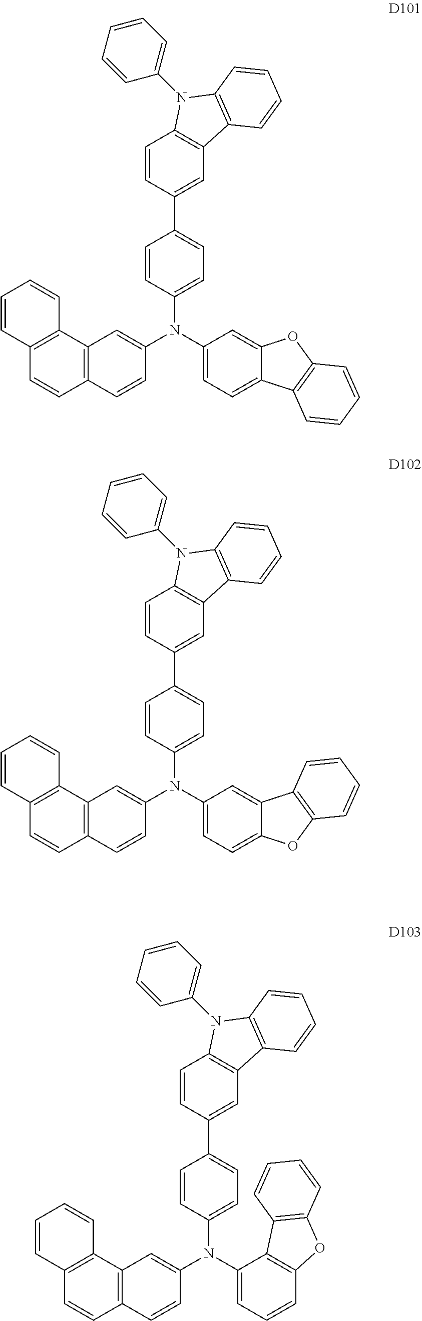 [SMILES: c1ccc(-n2c3ccccc3c3cc(-c4ccc(N(c5ccc6c(c5)oc5ccccc56)c5ccc6ccc7ccccc7c6c5)cc4)ccc32)cc1 ; c1ccc(-n2c3ccccc3c3cc(-c4ccc(N(c5ccc6ccc7ccccc7c6c5)c5ccc6oc7ccccc7c6c5)cc4)ccc32)cc1 ; c1ccc(-n2c3ccccc3c3cc(-c4ccc(N(c5ccc6ccc7ccccc7c6c5)c5cccc6oc7ccccc7c56)cc4)ccc32)cc1] 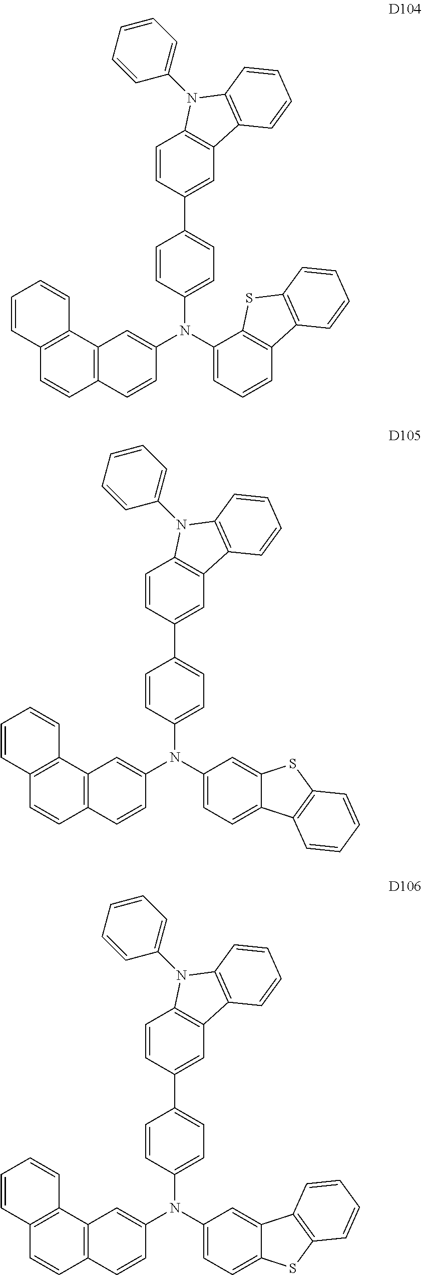 [SMILES: c1ccc(-n2c3ccccc3c3cc(-c4ccc(N(c5ccc6ccc7ccccc7c6c5)c5cccc6c5sc5ccccc56)cc4)ccc32)cc1 ; c1ccc(-n2c3ccccc3c3cc(-c4ccc(N(c5ccc6c(c5)sc5ccccc56)c5ccc6ccc7ccccc7c6c5)cc4)ccc32)cc1 ; c1ccc(-n2c3ccccc3c3cc(-c4ccc(N(c5ccc6ccc7ccccc7c6c5)c5ccc6sc7ccccc7c6c5)cc4)ccc32)cc1] 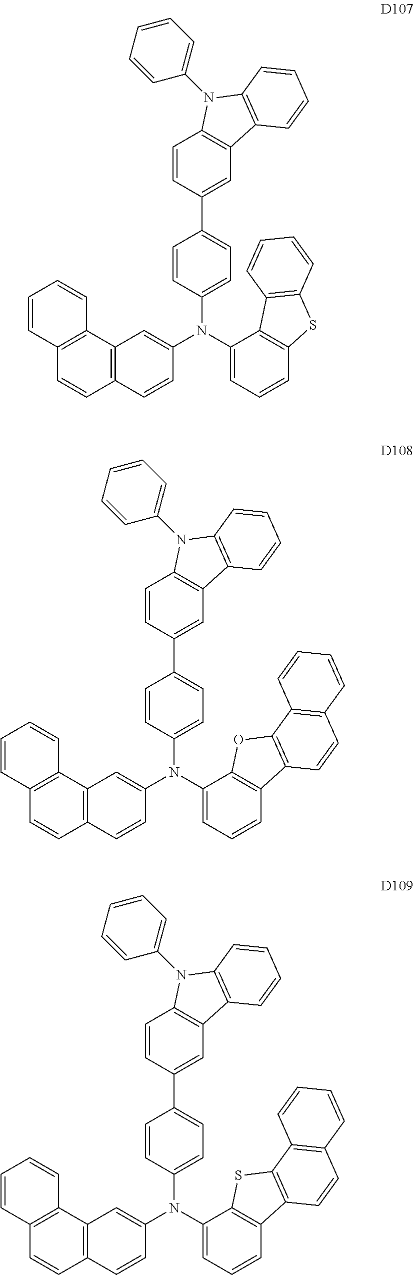 [SMILES: c1ccc(-n2c3ccccc3c3cc(-c4ccc(N(c5ccc6ccc7ccccc7c6c5)c5cccc6sc7ccccc7c56)cc4)ccc32)cc1 ; c1ccc(-n2c3ccccc3c3cc(-c4ccc(N(c5ccc6ccc7ccccc7c6c5)c5cccc6c5oc5c7ccccc7ccc65)cc4)ccc32)cc1 ; c1ccc(-n2c3ccccc3c3cc(-c4ccc(N(c5ccc6ccc7ccccc7c6c5)c5cccc6c5sc5c7ccccc7ccc65)cc4)ccc32)cc1] 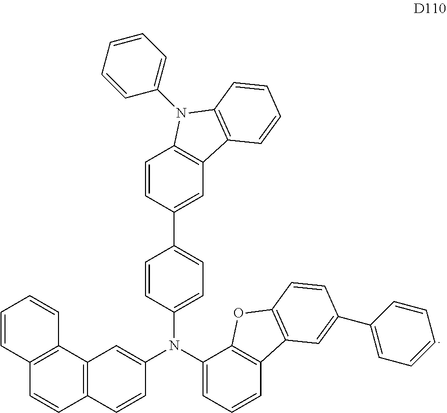 [SMILES: c1ccc(-c2ccc3oc4c(N(c5ccc(-c6ccc7c(c6)c6ccccc6n7-c6ccccc6)cc5)c5ccc6ccc7ccccc7c6c5)cccc4c3c2)cc1]
